(19) United States
(12) Patent Application Publication
(10) Pub. No.: US 2013/0156795 A1
(43) Pub. Date: Jun. 20, 2013

(54) METHODS FOR INHIBITION OF CELL PROLIFERATION, SYNERGISTIC TRANSCRIPTION MODULES AND USES THEREOF

(71) Applicants: Antonio Iavarone, New York, NY (US); Andrea Califano, New York, NY (US)

(72) Inventors: Antonio Iavarone, New York, NY (US); Andrea Califano, New York, NY (US)

(21) Appl. No.: 13/409,998

(22) Filed: Mar. 1, 2012

Related U.S. Application Data
(63) Continuation-in-part of application No. PCT/US2010/047556, filed on Sep. 1, 2010.
(60) Provisional application No. 61/238,964, filed on Sep. 1, 2009.
(60) Provisional application No. 61/244,816, filed on Sep. 22, 2009.
(60) Provisional application No. 61/294,190, filed on Jan. 12, 2010.

(51) Int. Cl. A61K 31/4015; A61K 31/4025; A61K 31/424; A61K 31/55; A61K 31/403; A61K 31/5377; A61K 31/47; A61K 31/136; A61K 31/426; A61K 31/4365; A61K 31/505; A61K 31/4985; A61K 31/437; A61K 31/555; A61K 31/473; A61K 31/4245; A61K 31/381; A61K 31/4035; A61K 31/4741; A61K 31/7048; A61K 31/513; A61K 38/45; A61K 31/4439; A61K 31/5395; A61K 31/506; A61K 31/517; A61K 31/472; A61K 31/4166; A61K 31/5375; A61K 31/444; A61K 31/496; A61K 31/435; A61K 31/498; A61K 31/515; C12Q 1/68; G01N 33/574; C40B 30/02; A61K 31/713; A61K 31/404

(52) U.S. Cl. CPC A61K 31/4015; A61K 31/404; A61K 31/4025; A61K 31/424; A61K 31/55; A61K 31/403; A61K 31/5377; A61K 31/47; A61K 31/136; A61K 31/426; A61K 31/4365; A61K 31/505; A61K 31/4985; A61K 31/437; A61K 31/555; A61K 31/473; A61K 31/4245; A61K 31/381; A61K 31/4035; A61K 31/4741; A61K 31/7048; A61K 31/513; A61K 38/45; A61K 31/4439; A61K 31/5395; A61K 31/506; A61K 31/517; A61K 31/472; A61K 31/4166; A61K 31/5375; A61K 31/444; A61K 31/496; A61K 31/435; A61K 31/498; A61K 31/515; C12Q 1/6837; C12Q 1/686; G01N 33/57407; C40B 30/02; A61K 31/713; C12Q 1/6886; C12Q 1/6897
USPC 424/174.1

(57) ABSTRACT

The invention provides for methods for treating nervous system cancers in a subject. The invention further provides methods for treating nervous system tumor cell invasion, migration, proliferation, and angiogenesis associated with nervous system tumors.

This application is a continuation-in-part of International Application PCT/US2010/047556, filed on Sep. 1, 2010, which claims priority to U.S. Provisional Application Nos. 61/238,964, filed on Sep. 1, 2009; 61/244,816, filed on Sep. 22, 2009; and 61/294,190, filed Jan. 12, 2010, the contents of each of which are incorporated herein by reference in their entireties.

### GOVERNMENT SUPPORT

The work described herein was supported in whole, or in part, by National Cancer Institute Grant Nos. R01-CA85628 and R01-CA101644, National Institute of Allergy and Infectious Diseases grant No. R01-A1066116, and National Centers for Biomedical Computing NIH Roadmap Initiative grant No. U54CA121852. Thus, the United States Government has certain rights to the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Glioma is a lethal disease with multiple genetic and epigenetic alterations. These changes work in concert in a coordinated fashion in cancer development and progression. Cancer Systems Biology is an emerging discipline in which high throughput genomic data and computational approaches are integrated to provide a coherent and systematic understanding of the diverse pathway dysregulations responsible for the presentation of the same cancer phenotype. This new discipline promises to transform the practice of medicine from a reactive one to a predictive one.

High-grade gliomas are the most common form of brain cancer, or brain tumors in human beings. Brain tumors are treated similarly to other forms of tumors with surgery, chemotherapy, and radiation therapy. There are relatively few specific drugs that selectively target tumors, and fewer still that target brain tumors. Here is described a pair of genes that appear to be responsible for the development of high-grade gliomas in humans. This pair of genes, Stat3 and C/EBPβ, can be used in a diagnostic, and serve as potential drug targets for the treatment of high-grade gliomas.

### SUMMARY OF THE INVENTION

An aspect of the invention provides a method for treating nervous system cancer in a subject in need thereof comprising administering to the subject a compound that inhibitis a Mesenchymal-Gene-Expression-Signature (MGES) protein.

An aspect of the invention provides a method for decreasing MGES protein activity in a subject having a nervous system cancer, the method comprising administering to the subject a compound that inhibits a MGES protein.

An aspect of the invention provides a method for inhibiting a MGES protein comprising contacting said protein with an effective amount of a MGES inhibitor compound.

An aspect of the invention provides a method for inhibiting tumor growth comprising contacting said protein with an effective amount of a MGES inhibitor compound.

An aspect of the invention provides a method for inhibiting cell proliferation comprising contacting said protein with an effective amount of a MGES inhibitor compound.

An aspect of the invention provides a method for detecting the presence of or a predisposition to a nervous system cancer in a human subject. In some embodiments, the method comprises (a) obtaining a biological sample from a subject; and (b) detecting whether or not there is an alteration in the expression of a Mesenchymal-Gene-Expression-Signature (MGES) gene in the subject as compared to a subject not afflicted with a nervous system cancer. In some embodiments, the MGES gene comprises Stat3, C/EBPβ, C/EBδ, RunX1, FosL2, bHLH-B2, ZNF238, or a combination thereof. In some embodiments, the detecting comprises detecting in the sample whether there is an increase in a MGES mRNA, a MGES polypeptide, or a combination thereof. In some embodiments some embodiments, the MGES gene comprises Stat3, C/EBPβ, C/EBPδ, RunX1, FosL2, bHLH-B2, or a combination thereof. In some embodiments, the detecting comprises detecting in the sample whether there is a decrease in a MGES mRNA, a MGES polypeptide, or a combination thereof. In some embodiments, the MGES gene comprises ZNF238. In some embodiments, the nervous system cancer comprises a glioma while in other embodiments, the glioma comprises an astrocytoma, a Glioblastoma Multiforme, an oligodendroglioma, an ependymoma, or a combination thereof.

An aspect of the invention provides a method for inhibiting proliferation of a nervous system tumor cell or for promoting differentiation of a nervous system tumor cell. In some embodiments, the method comprises decreasing the expression of a Mesenchymal-Gene-Expression-Signature (MGES) molecule in a nervous system tumor cell, thereby inhibiting proliferation or promoting differentiation. In some embodiments, the proliferation comprises cell invasion, cell migration, or a combination thereof. In some embodiments, the method comprises treatment of a subject in need thereof with a compound or composition that modulates MGES activity.

An aspect of the invention provides a method for inhibiting angiogenesis in a nervous system tumor, comprising administering to the subject an effective amount of a compound or composition. In some embodiments, the method comprises decreasing the expression of a Mesenchymal-Gene-Expression-Signature (MGES) molecule in a nervous system tumor cell, thereby inhibiting angiogenesis. In some embodiments, the method comprises treatment of a subject in need thereof with a compound or composition that modulates MGES activity.

Another aspect of the invention provides a method for treating a nervous system tumor in a subject, comprising administering to the subject an effective amount of a compound or composition that decreases the expression of a Mesenchymal-Gene-Expression-Signature (MGES) molecule in a nervous system tumor cell, thereby treating nervous system tumor in the subject. In some embodiments, the composition is administered to a nervous system tumor cell.

Another aspect of the invention provides a method for inhibition of an MGES protein in a subject, comprising administering to the subject an effective amount of a compound or composition that inhibits the activity of a MGES protein.

An aspect of the invention also provides a method for identifying a compound that binds to a Mesenchymal-Gene-Expression-Signature (MGES) protein. In some embodiments, the method comprises a) providing an electronic library of test compounds; b) providing atomic coordinates for at least 20 amino acid residues for the binding pocket of the MGES protein, wherein the coordinates have a root mean square deviation therefrom, with respect to at least 50% of Cα atoms, of not greater than about 5 Å, in a computer readable format; c) converting the atomic coordinates into electrical signals readable by a computer processor to generate a three dimensional model of the MGES protein; d) performing a data processing method, wherein electronic test compounds from the library are superimposed upon the three dimensional model of the MGES protein; and e) determining which test compound fits into the binding pocket of the three dimensional model of the MGES protein, thereby identifying which compound binds to the Mesenchymal-Gene-Expression-Signature (MGES) protein. In some embodiments, the method further comprises f) obtaining or synthesizing the compound determined to bind to the Mesenchymal-Gene-Expression-Signature (MGES) protein or to modulate MGES protein activity; g) contacting the MGES protein with the compound under a condition suitable for binding; and h) determining whether the compound modulates MGES protein activity using a diagnostic assay. In some embodiments, the MGES protein comprises Stat3, C/EBPβ, C/EBPδ, RunX1, FosL2, bHLH-B2, ZNF238. In some embodiments, the compound is a MGES antagonist or MGES agonist. In some embodiments, the antagonist decreases MGES protein or RNA expression or MGES activity by at least about 10%, at least about 20%, at least about 30%, at least about 40%, at least about 50%, at least about 60%, at least about 70%, at least about 75%, at least about 80%, at least about 90%, at least about 95%, at least about 99%, or 100%. In some embodiments, the antagonist is directed to Stat3, C/EBIβ, C/EBPδ, RunX1, FosL2, bHLH-B2 or a combination thereof. In some embodiments, the agonist increases MGES protein or RNA expression or MGES activity by at least about 10%, at least about 20%, at least about 30%, at least about 40%, at least about 50%, at least about 60%, at least about 70%, at least about 75%, at least about 80%, at least about 90%, at least about 95%, at least about 99%, or 100%. In some embodiments, the agonist is directed to ZNF238.

An aspect of the invention further provides for a compound identified by the screening method discussed herein, wherein the compound binds to MGES. In some embodiments, the compound binds to the active site of MGES.

An aspect of the invention also provides a method for decreasing MGES gene expression in a subject having a nervous system cancer, wherein the method comprises administering to the subject an effective amount of a composition comprising a MGES inhibitor compound, thereby decreasing MGES expression in the subject. In some embodiments, the composition comprises an MGES modulator compound. In some embodiments, the compound comprises an antibody that specifically binds to a MGES protein or a fragment thereof; an antisense RNA or antisense DNA that inhibits expression of MGES polypeptide; a siRNA that specifically targets a MGES gene; a shRNA that specifically targets a MGES gene; or a combination thereof.

An aspect of the invention further provides for a diagnostic kit for determining whether a sample from a subject exhibits increased or decreased expression of at least 2 or more MGES genes (e.g., Stat3, C/EBPβ, C/EBPδ, RunX1, FosL2, bHLH-B2, ZNF238), the kit comprising nucleic acid primers that specifically hybridize to an MGES gene, wherein the primer will prime a polymerase reaction only when a nucleic acid sequence comprising any one of SEQ ID NOS: 232, 234, 236, 238, 240, 242, or 244 is present.

In some embodiments, the compound is selected from the group consisting of etoposide, 5-fluorouracil, Clostridium difficile Toxin B,

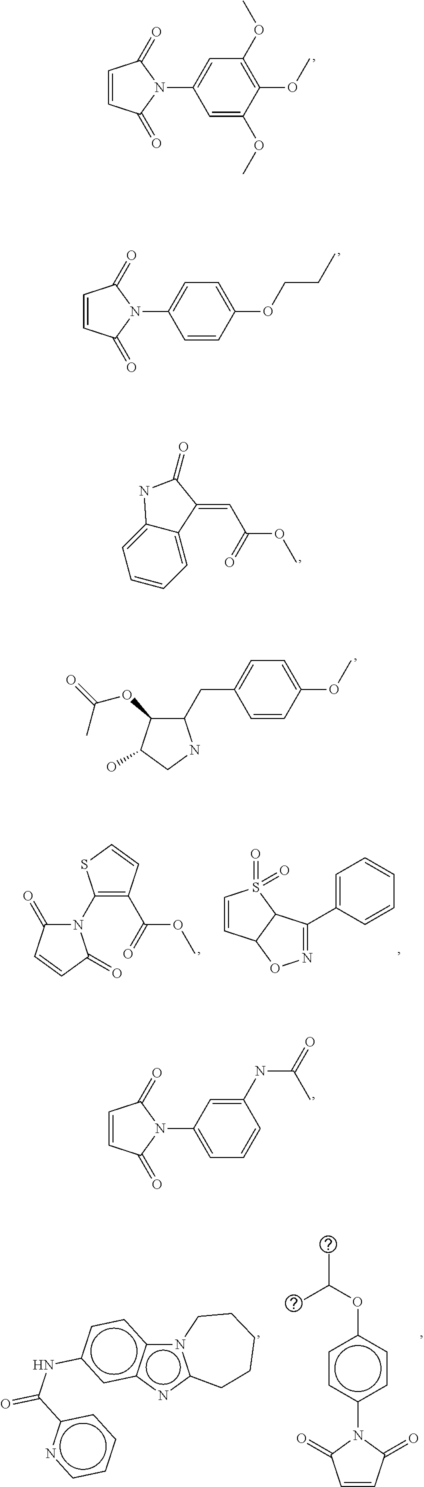

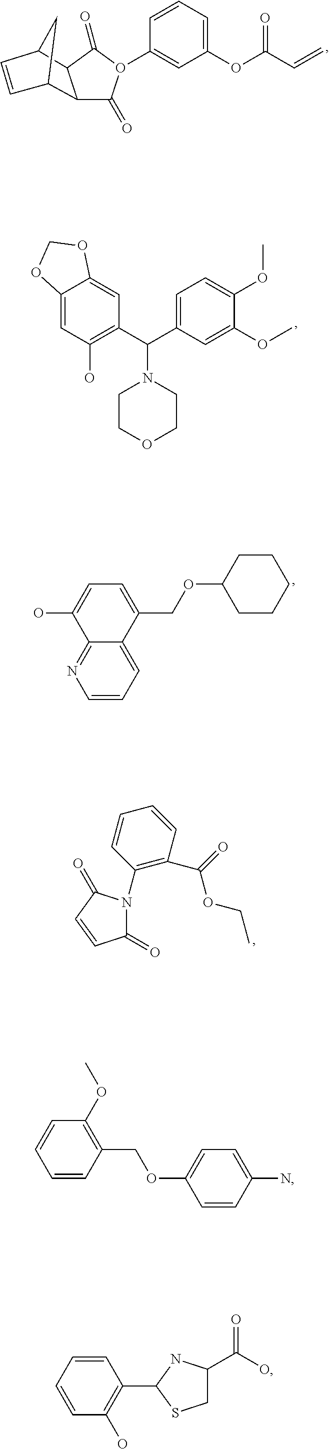

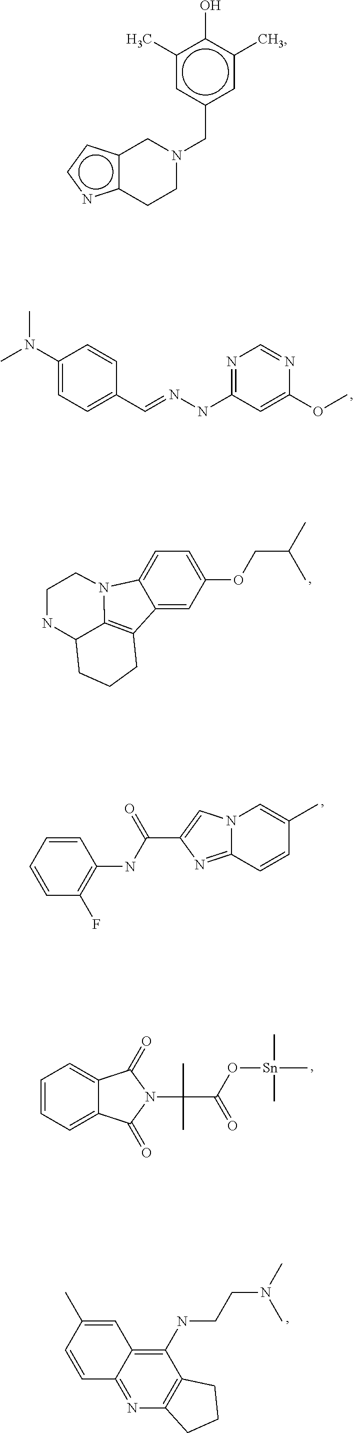

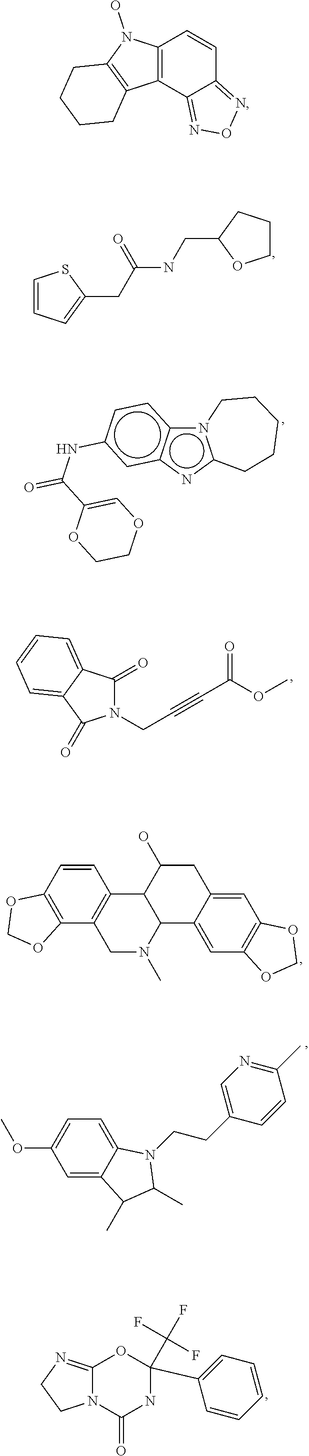

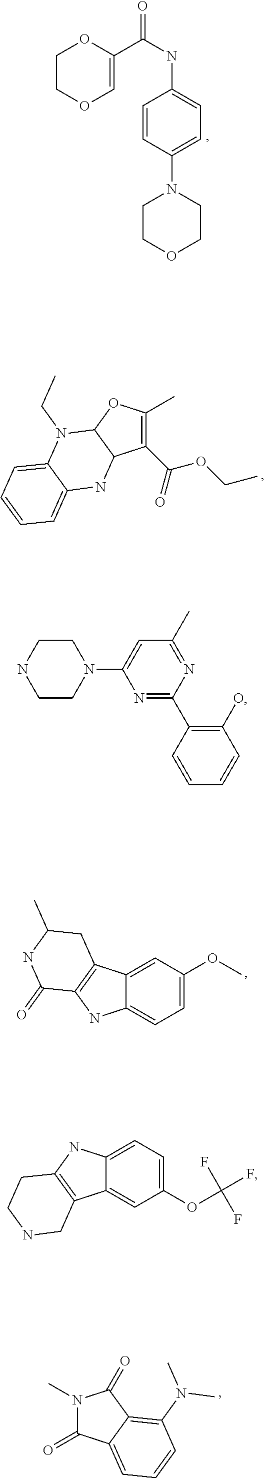

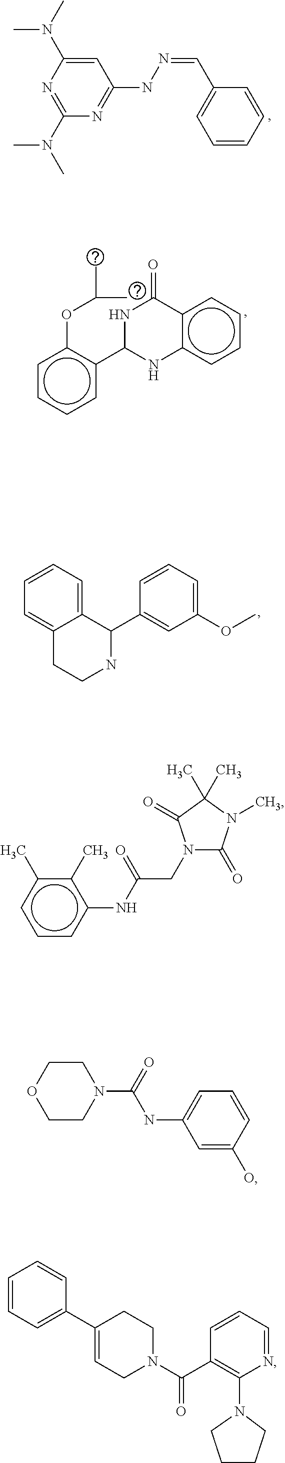

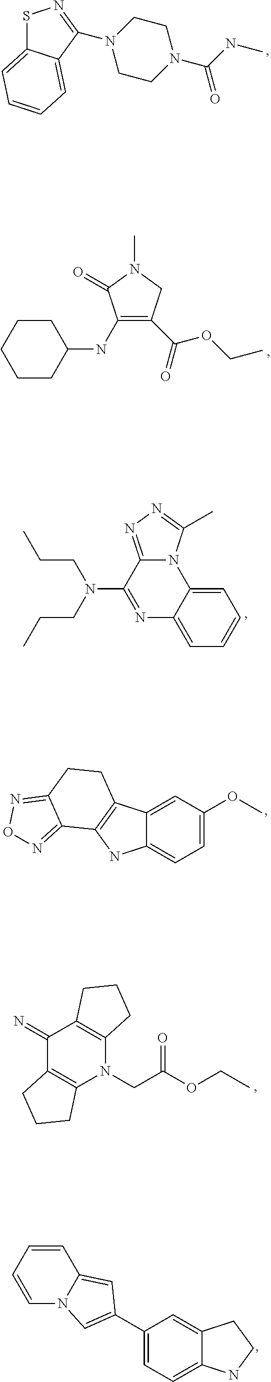

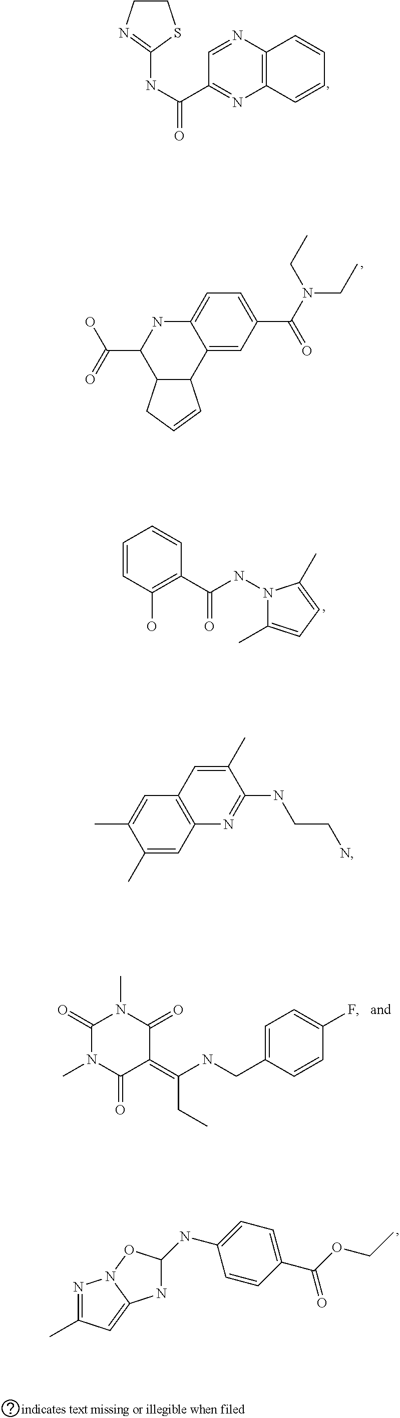

and pharmaceutically acceptable salts thereof.

In some embodiments, the composition, comprises a compound selected from the group consisting of etoposide, 5-fluorouracil, Clostridium difficile Toxin B,

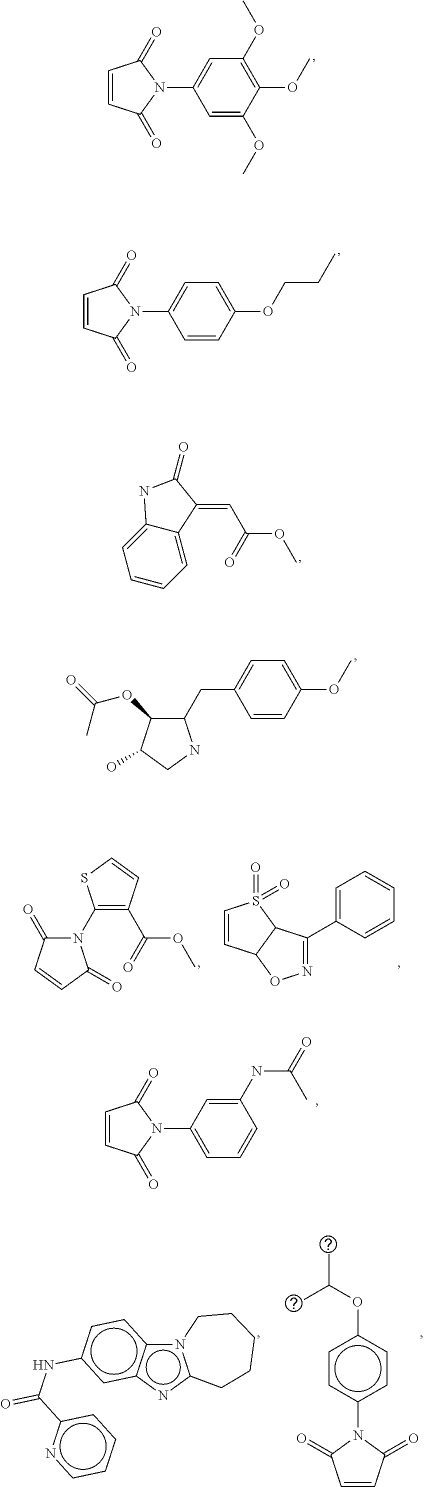

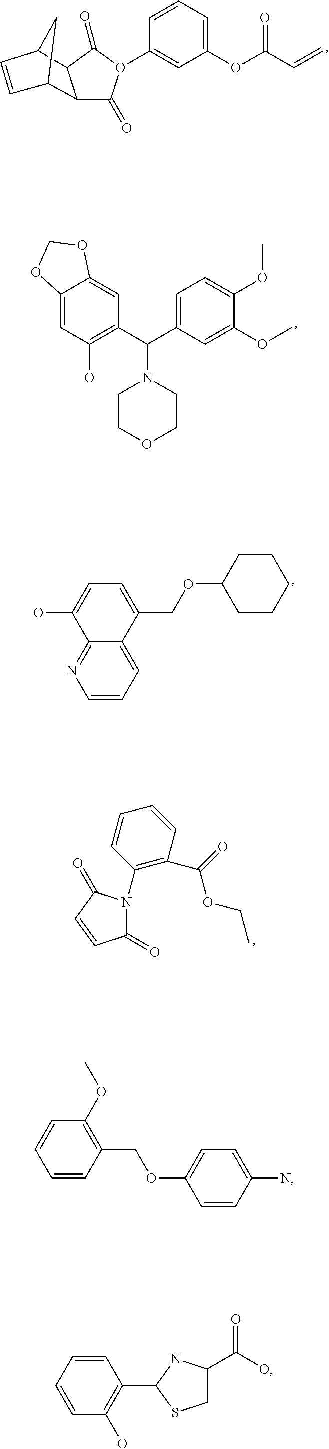

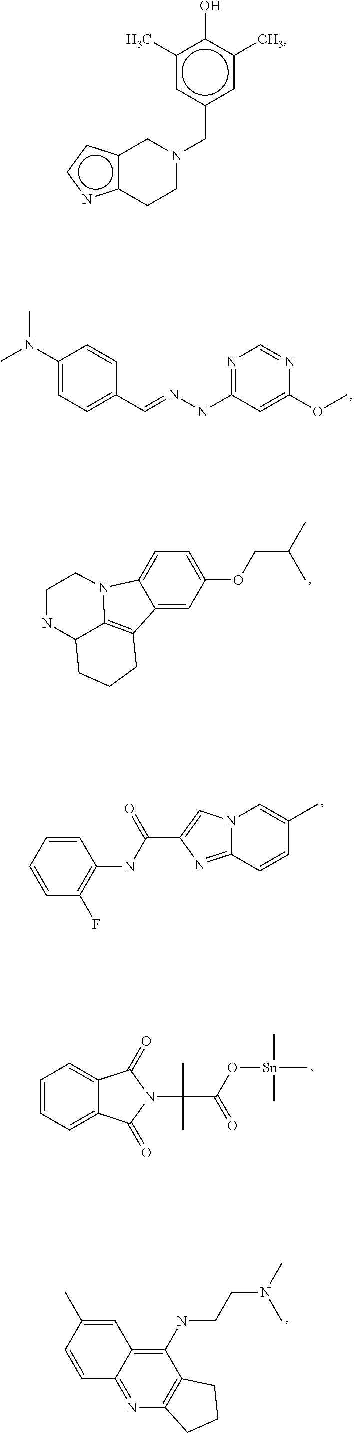

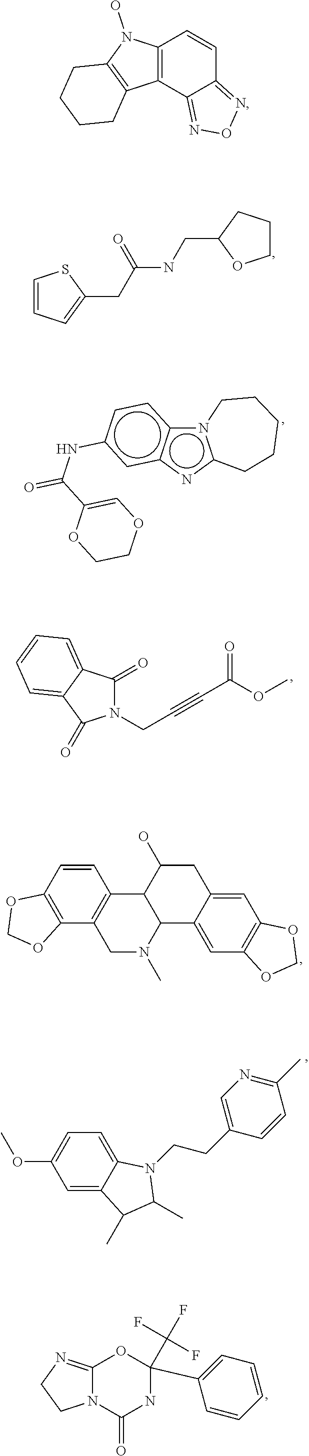

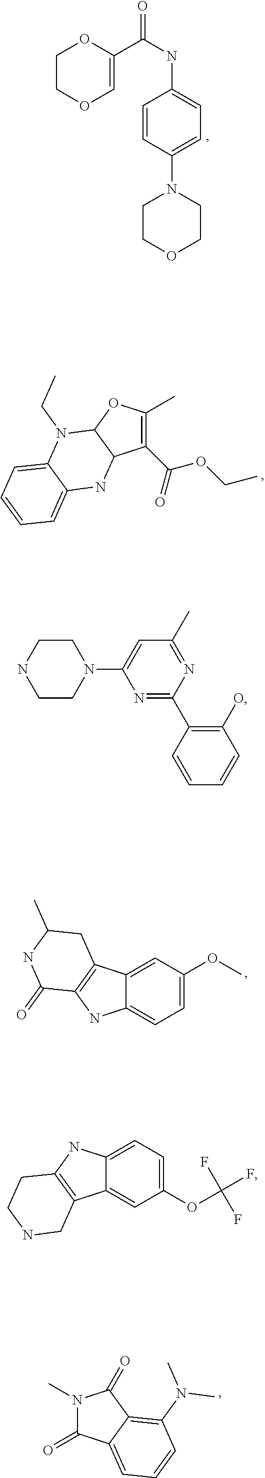

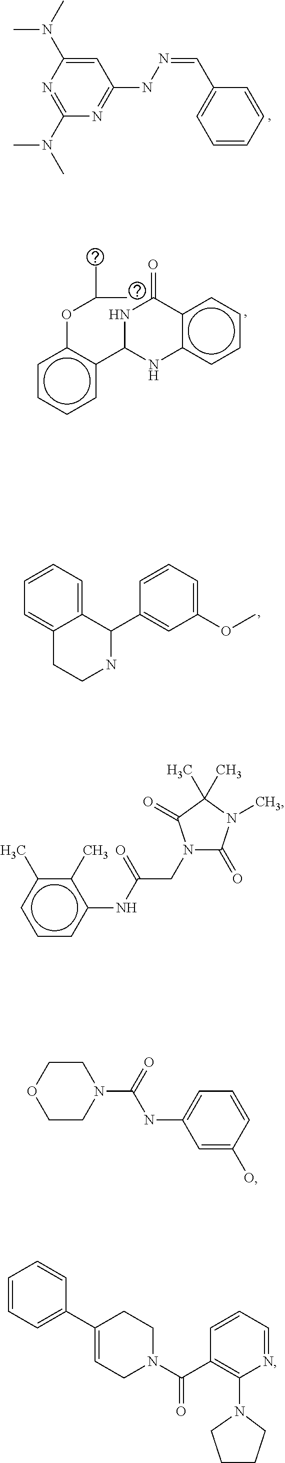

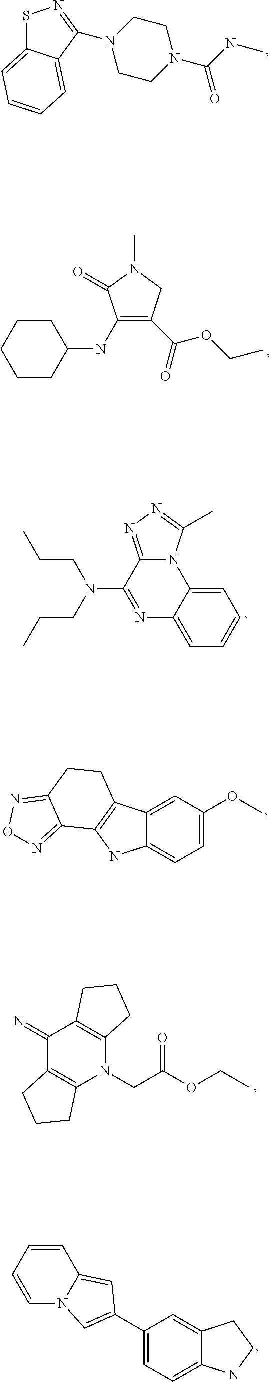

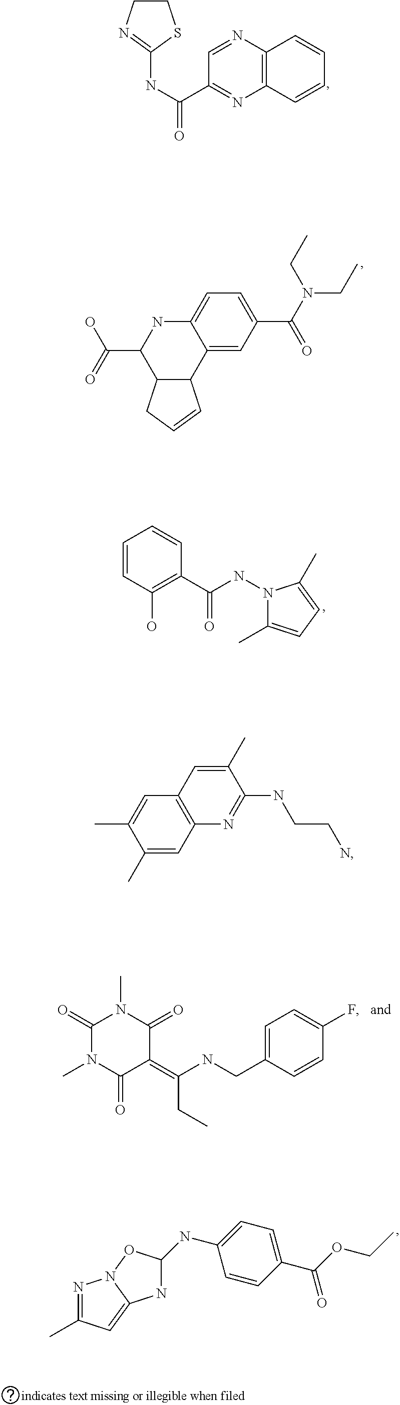

and pharmaceutically acceptable salts thereof.

In some embodiments, the MGES protein is C/EPB or Stat3. In some embodiments, the MGES protein is C/EPB. In some embodiments, the MGES protein is Stat3.

In some embodiments, the cancer is glioma or meningioma. In some embodiments, the cancer is astrocytoma, Glioblastoma Multiforme, oligodentroglioma, ependymoma or meningioma. In some embodiments, the cancer is cerebellar astrocytoma, medulloblastoma, ependymona, brain stem glioma, optic nerve glioma, acoustic neuromas, nerve sheath tumors, or germinoma.

These and other embodiments of the invention are further described in the following sections of the application, including the Detailed Description, Examples, and Claims. Still other objects and advantages of the invention will become apparent by those of skill in the art from the disclosure herein, which are simply illustrative and not restrictive. Thus, other embodiments will be recognized by the ordinarily skilled artisan without departing from the spirit and scope of the invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a schematic depicting the mesenchymal subnetwork of six major hubs of transcription factors (TFs) in high-grade gliomas which represents the mesenchymal signature of high-grade gliomas is controlled by six TFs. The TFs positively (pink) or negatively (blue) linked as first neighbors to the mesenchymal genes of human gliomas (green) connect 74% of the genes composing the MGES. The six TF control 74% of the genes in the mesenchymal signature of high-grade glioma.

FIG. 2 is a photographic representation of a blot showing expression of the TFs connected with the MGES in primary GBM. Semiquantitative RT-PCR was performed in 17 GBM samples, in the SNB75 glioblastoma cell line and normal brain. 18S RNA was used as control.

FIG. 3 shows the validation of direct targets of the TFs connected with the MGES by ChIP analysis. A region between 2 kb upstream and downstream the transcription start of the targets identified with ARACNe was analyzed for the presence of putative binding sites. Genomic regions of genes containing putative binding sites for specific TFs were immunoprecipitated in the SNB75 cell line by antibodies specific for Stat3 (FIG. 3A), C/EBPβ (FIG. 3B), FosL2 (FIG. 3C), and bHLH-B2 (FIG. 3D). SOCS3 was included as positive control of Stat3 binding. Total chromatin before immunoprecipitation (input DNA) was used as positive control for PCR. The OLR1 gene was used as a negative control. FIG. 3E shows the summary of binding results of the tested TFs to mesenchymal targets.

FIG. 4 shows a combinatorial and hierarchical module directs interactions between the master mesenchymal TFs. The promoters of the TFs connected to the MGES were analyzed for the presence of putative binding sites for Stat3 (FIG. 4A), C/EBPβ (FIG. 4B), FosL2 (FIG. 4C), and bHLHB2 (FIG. 4D) through the MatInspector software (Genomatix) followed by ChIP. FIG. 4E shows a graphical representation of the transcriptional network emerging from promoter occupancy analysis, including autoregulatory and feed-forward loops among TFs. FIG. 4F shows quantitative RT-PCR analysis of mesenchymal TFs in GBM-BTSCs infected with lentivirus expressing Stat3/C/EBPβ shRNA. Gene expression is normalized to the expression of 18S ribosomal RNA.

FIG. 5A shows photographic images of the morphology of Stat3 plus C/EBPβ-expressing clones grown in the presence and absence of mitogens. Ectopic Stat3C and C/EBPβ in NSCs induce a mesenchymal phenotype, enhance migration and invasion and inhibit proneural gene expression.

FIG. 5B shows Gene Set Enrichment Analysis plots. Following ectopic expression of C/EBPβ and Stat3 in NCSs, mesenchymal (mes) and proliferative (prolif) genes were highly enriched among upregulated genes, while the proneural (PN) genes were highly enriched among down-regulated genes. Top portion of the graph shows the enrichment score profile. The maximum (minimum) value of this curve determines the enrichment score among up-regulated (down-regulated) genes. Middle portion of the graph shows the signature genes as black vertical bars. The bottom portion shows the weight of each ranked gene (proportional to its statistical significance). The figure is separated into two pages, joining at the hatched line.

FIG. 5C are microphotographs of C17.2 expressing Stat3C and C/EBPβ or the empty vector. 1 mm scratch was made with a pipette tip on confluent cultures (upper panels): The ability of the cells to cover the scratch was evaluated after three days (lower panels). *p≦0.05, **p≦0.01.

FIG. 5D shows microphotographs of invading C17.2 cells expressing Stat3C and C/EBPβ or transduced with empty vector (upper panels). Quantification of cell invasion in the absence or in the presence of PDGF. Bars indicate Mean±SEM of triplicate samples. *p≦0.05, **p≦0.01.

FIG. 6 depicts that neural stem cells expressing Stat3C and C/EBPβ acquire tumorigenic capability in vivo. FIG. 6A shows six-week old BALBc/nude mice that were injected subcutaneously with C17.2-vector (left flank) or C17.2 expressing Stat3C plus C/EBPβ (right flank). The number of tumors observed is indicated in the table. Mice were sacrificed 10 weeks (5×10⁶ cells) or 13 weeks (2.5×10⁶ cells) after injection. Black arrows point to the normal appearance of the left flank injected with CTR cells. White arrows point to the tumor mass in the right flank injected with C17.2 expressing Stat3C plus C/EBPβ. FIG. 6B are photographs of Hematoxylin & Eosin staining of two representative tumors depicting areas of pleomorphic cells forming pseudopalisades (upper panels; Inset: N, necrosis) and intensive network of aberrant vascularization (lower panels). FIG. 6C are photographic microscopy images of tumors that exhibit immunopositive areas for the proliferation marker Ki67, the progenitor marker Nestin, and diffuse staining for the vascular endothelium as evaluated by CD31. FIG. 6D are photographic microscopy images of tumors that display mesenchymal markers as indicated by positive immunostaining for OSMR and FGFR-1. Two representative tumors are shown.

FIGS. 7A-7B show expression of Stat3 and C/EBPβ is essential for the mesenchymal phenotype of human glioma. FIG. 7A is a photographic image of a western blot of Stat3 and C/EBPβ in brain tumor stem cells (BTSCs) transduced with lentivirus CTR or expressing Stat3 and C/EBPβ shRNA. FIG. 7B is a graphic representation of the GSEA plot for the mesenchymal genes.

FIG. 7C is a bar graph that shows quantitative RT-PCR of mesenchymal genes in BTSCs infected with lentiviruses expressing Stat3/C/EBPβ shRNA. Gene expression is normalized to the expression of 18S rRNA.

FIG. 7D is a graphic representation of a GSEA plot. The MGES is downregulated in SNB19 cells infected with shStat3 plus shC/EBPβ silencing lentiviruses.

FIG. 7E shows photographic images of invading SNB19 cells infected with shStat3 plus shC/EBPβ lentiviruses. The graph shows Mean+/−SD of two independent experiments, each performed in triplicate.

FIG. 7F is a graph depicting Kaplan-Meier survival of patients carrying tumors positive for Stat3 and C/EBPβ (double positives, red line) and double/single negative tumors (black line).

FIG. 8 depicts that MINDy-inferred STK38 is a post-translational modulator of MYC. (FIG. 8A) rows represent MYC targets, columns represent distinct samples. Expression is color coded from blue (underexpressed) to red (overexpressed) with respect to the mean across all experiments. MYC ability to transcriptionally regulate its targets is reduced in samples with lower STK38 expression. Silencing of STK38 leads to reduction in MYC protein (FIG. 8B), consistent changes in validated MYC targets (FIG. 8C), but no change in MYC mRNA (FIG. 8C)

FIG. 9 is a graph that shows the expression of ZNF238 is significantly down-regulated in 77 samples from human GBM (class 2, red) compared with 23 samples from non-tumor human brains (class 1, blue). P-value: 6.8E-5.

FIG. 10 is a graph that shows expression of ZNF238 in tumors derived from NCS expressing Stat3/C/EBPβ. RNA was prepared from cells before injection and two representative tumors. Quantitative RT-PCR was performed using 18S as internal control.

FIG. 11 is a bar graph that shows SiRNA-mediated silencing of ZNF238 in NSCs expressing Stat3 and C/EBPβupregulates the expression of mesenchymal genes.

FIG. 12 shows graphs that depict results from epigenetic silencing of ZNF238 in malignant glioma cells. FIG. 12A, Graphical representation of the promoter of ZNF238. The region between −1800 and −3400 contains stretches of CpG islands. FIG. 12B, 5-Azacytidine induces expression of ZNF238. T98G cells were treated with 5-Azacytidine at the indicated concentrations for 3 days. Expression of ZNF238 was analyzed by quantitative PCR. FIG. 12C, Expression of selected ZNF238 targets is down-regulated after treatment with 5-Azacytidine. HPRT was used as control for normalization.

FIG. 13 is a schematic for the generation of mice carrying conditional inactivation of the ZNF238 gene. A 10.3 Kb genomic fragment containing ZNF238 locus has been retrieved into PL253 plasmid by recombineering using the recombination proficient bacterial strain SW102, which expresses the recombinase components exo, bet, and gam. A loxP site will be introduced in intron 1, upstream of the ZNF238 coding region. A loxP-flanked Neo-STOP cassette (LSL) from pBS302 vector will be introduced into the 3′ untranslated region of exon 2 by recombineering. The LSL cassette was obtained from Tyler Jacks. The linearized targeting vector will be introduced into ES cells by electroporation. Deletion of the coding region in exon 2 by Cre in vivo will generate ZNF238-null mice.

FIG. 14 depicts GEP profiles from the Glioma Connectivity Map will be used to prioritize candidate druggable targets for MGES inhibition. For each Candidate Pharmacological Target (CPT), samples will be sorted by CPT expression. Enrichment of the MGES in genes that are differentially expressed in the GEPs that express the highest/lowest CPT levels will be used to assess the likelihood that the CPT is effective in suppressing the MGES.

FIG. 15 is a fluorescent photographic image depicting the silencing of Stat3 and C/EBPβ in human GBM-BTSCs induces apoptosis. Cells transduced with sh-CTR or sh-Stat3 plus sh-C/EBPβ. Cells were immunostained for Caspase3. Nuclei were counterstained with DAPi.

FIG. 16 is a photograph of a blot showing chromatin immunoprecipitation for Stat3 (FIG. 16A) and C/EBPβ (FIG. 16B) from a primary GBM sample.

FIG. 17 shows that ectopic expression of C/EBPβ and Stat3C cooperatively induce the expression of mesenchymal markers in NSCs. FIG. 17A is a photographic image of a western blot. FIG. 17B shows Immunofluorescence staining for SMA (upper panel) and fibronectin (lower panel) in C17.2 expressing the indicated TFs. FIG. 17C depicts the quantification of SMA positive cells (upper panel). For fibronectin immunostaining the intensity of fluorescence was quantified (lower panel). Bars indicate Mean±SD. n=3 for each group. **p≦0.01, ***p≦0.001. FIG. 17D-G shows the QRT-PCR analysis of mesenchymal targets in C17.2 expressing the indicated TFs or transduced with the empty vector. Gene expression was normalized to the expression of 18S ribosomal RNA. Bars indicate Mean±SD. n=3 for each group. **p≦0.01, ***p≦0.001.

FIG. 18 shows that C/EBPβ and Stat3 inhibit neural differentiation of NSCs, induce mesenchymal transformation and promote invasiveness. FIG. 18A is a photographic image of a semi-quantitative RT-PCR analysis of mesenchymal and neural markers in C17.2 expressing Stat3C plus C/EBPβ or control vector cultured in growth medium (E) or after removal of mitogens for 5 or 10 days. FIG. 18B are microscope photographs of Alcian blue staining of C17.2 expressing Stat3C and C/EBPβ, or transduced with empty vector cultured in growth medium (upper panels), or in chondrogenesis differentiation medium for 20 days (lower panels).

FIG. 19 shows that C/EBPβ and Stat3 inhibit neural differentiation and trigger mesenchymal transformation of primary mouse NSCs. FIG. 19A are photomicrographs of immunofluorescence staining for CTGF in primary NSCs transduced with retroviruses expressing Stat3C and C/EBPβ or the empty vector. GFP identifies the infected cells. FIG. 19B is a graph showing the quantification of GFP positive/CTGF positive cells. Bars indicate Mean±SD of three independent experiments. **p≦0.01. FIG. 19C is a graph showing QRT-PCR of mesenchymal genes in primary N, SCs transduced with Stat3C, C/EBPβ, Stat3C plus C/EBPβ, or empty vectors. Bars indicate Mean±SD of 3 independent reactions. Gene expression was normalized to the expression of 18S ribosomal RNA. FIGS. 19D-F are graphs showing QRT-PCR of neuronal (βIII-tubulin and doublecortin) and glial (GFAP) markers in primary NSCs transduced with Stat3C plus C/EBPβ, or with empty retroviruses. Cells were grown for 5 days in the presence or absence of mitogens. Bars indicate Mean±SD of three independent reactions. Gene expression was normalized to the expression of 18S ribosomal RNA.

FIG. 20 shows that C/EBPβ and Stat3 are essential to maintain the mesenchymal phenotype of human glioma cells. FIG. 20A are microphotographs of immunofluorescence for fibronectin, Col5A1 and YKL40 in BTSC-3408 infected with lentiviruses expressing Stat3, C/EBPβ, or Stat3 plus C/EBPβ shRNA. Nuclei were counterstained with DAPI. Quantification of fibronectin (FIG. 20C), Col5A1 (FIG. 20D) and YKL40 (FIG. 20E) positive cells from the representative experiment shown in (FIG. 20A). Bars indicate Mean±SD of 3 independent experiments. *p≦0.05, **p≦0.01, ***p≦0.001. FIG. 20B are photomicrographs of immunofluorescence for Col5A1 and YKL40 in SNB19 cells infected as in FIG. 20A. Quantification of Col5A1 (FIG. 20F) and YKL40 (FIG. 20G) positive cells in experiments in (FIG. 20B). Bars indicate Mean±SD of 3 independent experiments. *p≦0.05, **p≦0.01. QRT-PCR of mesenchymal genes in BTSC-20 (FIG. 20H), BTSC-3408 (FIG. 20I) and SNB19 (FIG. 20J) infected with lentiviruses expressing Stat3, C/EBPβ, or Stat3 plus C/EBPβ shRNA. Bars indicate Mean±SD of three independent reactions. FIG. 20K is a bar graph showing the quantification of Stat3 plus C/EBPβ shRNA.

FIG. 21 shows that knockdown of C/EBPβ and Stat3 impairs tumor formation, invasion and expression of mesenchymal markers in a mouse model of human SNB19 glioma. FIG. 21A depicts a Kaplan-Meier survival curve of NOD SCID mice transplanted intracranially with SNB19 glioma cells that had been transduced with shCtr (red), shStat3 (black), shC/EBPβ (green) or shStat3 plus shC/EBPβ (blue) lentiviruses. **p≦0.01. Immunofluorescence staining for human Vimentin (FIG. 21B), CD31 (FIG. 21C), fibronectin (FIG. 21D), Col5A1 (FIG. 21E) and YKL40 (FIG. 21F) of tumors derived from SNB19 cells infected with lentiviruses expressing shRNA targeting Stat3, C/EBPβ, or Stat3 plus C/EBPβ. T, tumor; B, normal brain.

FIG. 22 shows that C/EBPβ and Stat3 are essential for glioma tumor aggressiveness in mice and humans. FIG. 22A depicts invading BTSC-3408 cells infected with shCtr, shStat3, shC/EBPβ or shStat3 plus shC/EBPβ lentiviruses and the quantification of invading cells (graph below). Bars indicate Mean±SD of two independent experiments, each performed in triplicate (right panel). *p≦0.01. FIG. 22B shows immunostaining for human vimentin (left panels) on representative brain sections from mice injected with BTSC-3408 after silencing of C/EBPβ and Stat3. Quantification of human vimentin positive area (right panel). FIG. 22C shows immunostaining for Ki67 from tumors as in FIG. 22B (left panels). Quantification of Ki67 positive cells (right panel). Bars indicate Mean±SD. n=5 for each group. *p≦0.05. (St, striatum; CC, corpus callosum). Immunostaining for fibronectin (FIG. 22D) and Col5A1 (FIG. 22E) on representative brain sections from mice injected with BTSC-3408 that had been transduced treated as indicated. Nuclei were counterstained with DAPI. f, Kaplan-Meier analysis comparing survival of patients carrying tumors positive for C/EBPβ and Stat3 (double positives, red line) and double/single negative tumors (black line).

FIG. 23 is a schematic that shows altered MGES gene expression does not result from copy number changes. The correlation between gene expression and DNA copy number for the MGES genes was determined using data from 76 high-grade gliomas for which both gene expression array (Affymetrix U133A) and array comparative genomic hybridization (aCGH) profiling has been performed as previously described{Phillips, 2006 #1049}. Tumors were grouped based on molecular subtype (proneural, mesenchymal, or proliferative) and the mean expression of each MGES gene determined. Genes are shown in order of increasing mean expression. The normalized copy number (error bars indicate standard deviation) of each gene was interpolated based on the copy number of the nearest genomic clone on the CGH array as determined by comparison of the sequence annotation of both array platforms. No correlation was seen between the mean MGES gene expression and DNA copy number for the proneural, mesenchymal, proliferative groups or the total cohort (p=0.09430, 0.1058, 0.09430, 0.1014, respectively; Spearman's rho).

FIG. 24 are graphs that show the correlation between microarray and QRT-PCR measures for Stat3 (FIG. 24A) and C/EBPβ (FIG. 24B) mRNAs. Shown is the ratio of mRNA levels for C/EBPβ and Stat3 between silencing or over-expression and the corresponding non-targeting shRNA or vector controls, respectively. QRT-PCR estimates α-axis) are in log₁₀ scale, and microarray estimates (y-axis) are in log₂ scale.

FIG. 25 is a graph of GSEA analysis that confirmed that MGES genes were markedly enriched in the TWPS signature. The bar-code plot indicates the position of the MGES genes on the TCGA expression data rank-sorted by its association with bad prognosis, red and blue colors indicate positive and negative differential expression, respectively. The gray scale bar indicates the t-statistic values, used as weighting score for GSEA analysis.

FIG. 26 shows ectopic Stat3C and C/EBPβ in NSCs induce a mesenchymal phenotype and inhibit neuronal differentiation. FIG. 26A shows immunofluorescence for Tau and SMA in two C17.2 subclones expressing Stat3C and C/EBP or control vector cultured in absence of mitogens for 10 days. Nuclei were counterstained with DAPI. FIG. 26B are microphotographs of primary mouse NSCs expressing Stat3C and C/EBPβ or control vector grown in absence of growth factors. Note the differentiated cells with neuronal-like morphology in the control cells.

FIG. 27 are photomicrographs that show YKL-40 expression correlates with C/EBPβ and Stat3 expression in primary tumors. Immunohistochemistry analysis of YKL-40, C/EBPβ and Stat3 expression in tumors from patients with newly diagnosed GBM. FIG. 27A shows a representative YKL-40/Stat3C/EBPβ-triple positive tumor. FIG. 27B shows a representative YKL-40/Stat3/C/EBPβ-triple negative tumor.

FIG. 28. is a graph showing change in gene expression.

FIG. 29 is a schematic that shows the top 50 genes downregulated (FIG. 29A) and the top 50 genes downregulated (FIG. 29B).

FIG. 30 shows chromatin immunoprecipitation for Stat3 and C/EBPβ (FIG. 30A) from primary GBM tumor samples and quantitation of their expression (FIG. 30B).

FIG. 31A is a venn-diagram that depicts the proportion of mesenchymal genes identified by ARACNe as targets of only C/EBPβ, STAT3 or both TFs.

FIG. 31B is a heatmap of MGES gene expression analysis of mouse and human cells carrying perturbations of C/EBPβ plus STAT3. Samples (columns) were grouped according to species and treatment. Control, control shRNA or empty vector; S−, STAT3 knockdown; S+, STAT3 overexpression; C−, CEBPB knockdown; C+, CEBPB overexpression; S−/C−, STAT3 and CEBPB knockdown; S+/C+, STAT3 and CEBPB overexpression.

FIG. 32 is a graph showing the GSEA of the MGES on the gene expression profile rank-sorted according to the correlation with the CEBPB×STAT3 metagene. The bar-code plot indicates the position of MGES genes, light gray (right hand side) and dark grey (left hand side) colors represent positive and negative correlation, respectively. The grey scale bar indicates the Spearman's rho coefficient used as weighting score for GSEA. LEOR, leading-edge odds ratio; nES, normalized enrichment score; P, sample-permutation-based P value

FIG. 33 is a schematic diagram of the experimental strategy used to identify and experimentally validate the transcription factors (TFs) that drive the mesenchymal phenotype of malignant glioma. Reverse-engineering of a high grade glioma-specific mesenchymal signature reveal the transcriptional regulatory module that activates expression of the mesenchymal genes. Two transcription factors (C/EBPβ and STAT3) emerge as synergistic master regulators of mesenchymal transformation. Elimination of the two factors in glioma cells leads to collapse of the mesenchymal signature and reduces tumor formation and aggressiveness in the mouse. In human glioma, the combined expression of C/EBPβ and STAT3 is a strong predicting factor for poor clinical outcome.

FIG. 34 shows that mesenchymal genes are coordinately regulated by C/EBPβ and Stat3. Gene expression integrative analysis of mouse and human cells carrying perturbations of C/EBPβ (FIG. 34A) and Stat3 (FIG. 34B). Heatmaps represent mRNA levels for MGES genes. Genes are in rows and samples in columns. The 89 profiled samples were grouped according to species and treatment: control shRNA or empty vector (Control), Stat3 knock-down (S−), Stat3 overexpression (S+), C/EBPβ knock-down (C−), C/EBPβ overexpressoin (C+), simultaneous knockdown or over-expression of both TFs (S−/C− and S+/C+). The first row of each heatmap shows the mRNA levels of C/EBPβ and Stat3 as assessed by qRT-PCR. Genes were sorted according to the Spearman correlation with the mRNA levels of the specific TF being tested. Dark grey and light gray intensity indicate lower and higher expression levels than the gene expression median, respectively. Leading edge mesenchymal genes are above the horizontal black line. GSEA analysis of the MGES on the gene expression profile rank-sorted is shown according to the correlation with C/EBPβ (FIG. 34C) and Stat3 (FIG. 34D). The bar-code plot indicates the position of the MGES genes, dark gray (left-hand side of the plot) and light gray (right-hand side of the plot) colors indicate positive and negative correlation, respectively. The gray scale bar indicates the spearman rho coefficient, used as weighting score for GSEA analysis. nES, normalized enrichment score; p, sample-permutation-based p-value.

FIG. 35 shows results from C/EBPβ and STAT3 luciferase reporter assays. TRANSIENT analysis of the reporters is shown in the bar graphs, Left Panel (STAT3, Top; and C/EBPβ, Bottom) and in the blots of expression, Middle Panel (STAT3, Top; and C/EBPβ, Bottom). A schematic of luciferase reporter vectors expressing STAT3 (Top) and C/EBPβ (Bottom) are shown in the right panel.

FIG. 36 shows expression levels of SNB19 human glioma cell clones that were stably transfected with the C/EBPbeta-driven luciferase plasmid and subsequently transfected with control siRNAs or siRNA oligonucleotides targeting C/EBPbeta.

FIG. 37 shows expression levels of SNB19 human glioma cell clones that were stably transfected with the C/EBPbeta-driven luciferase plasmid and subsequently transfected with control siRNAs or two different siRNA oligonucleotides targeting C/EBPbeta (siCEBPb05 and siCEBP06).

FIG. 38 shows inhibition using a C/EBPb gene reporter assay. FIG. 38A shows CEBPb reporter activity at 48 hr upon inhibition with various dosages of 5-fluorouracil (5-FU). FIG. 38B shows ATP cell viability at 24 hr and 48 hr upon inhibition with various dosages of 5-FU.

FIG. 39 shows inhibition using a C/EBPb gene reporter assay. FIG. 39A shows CEBPb reporter activity at 48 hr upon inhibition with various dosages of clostridium difficilis Toxin B (CD Toxin B). FIG. 39B shows ATP cell viability at 24 hr and 48 hr upon inhibition with various dosages of CD Toxin B.

### DETAILED DESCRIPTION OF THE INVENTION

Key features of nervous system cancer progression are relentless proliferation, loss of differentiation and angiogenesis (Iavarone and Lasorella, 2004. Cancer Letters 204: 189-96; herein incorporated by reference in its entirety). Here, the invention is directed to transcriptional modules that can synergistically initiate and maintain mesenchymal transformation in the brain. For example, the invention is directed to regulating the mesenchymal state of brain cells, a signature of human glioma. In some embodiments, transcription factors that comprise a transcriptional module involved in the synergistic regulation of the mesenchymal signature of malignant glioma (Mesenchymal Gene Expression Signature of high-grade glioma (MGES)) are regulated so as to reduce nervous system cancers. MGES genes can include, but are not limited to, e.g., Stat3, C/EBPβ, C/EBPδ, RunX1, FosL2, bHLH-B2, ZNF238, or a combination thereof. In some embodiments, the protein or mRNA expression levels of Stat3 and/or C/EBPβ can be decreased in order to ameliorate glioma cancers. For example, silencing of the two transcription factors depletes glioma stem cells and cell lines of mesenchymal attributes and greatly impairs their ability to invade.

The invention is also directed methods of inducing spinal axon regeneration by way of a stabilized Id2 composition. In some embodiments, the delivery of Adeno-Associated Viruses encoding undegradable Id2 (Id2-DBM) can promote axonal regeneration and functional locomotor recovery in a mouse model of hemisection spinal cord injury.

As used herein, “Mesenchymal Gene Expression Signature” or “MGES” refers to a transcription factor that comprises a transcriptional module involved in the synergistic regulation of the mesenchymal signature of malignant glioma or high-grade glioma. For example, MGES genes can include, but are not limited to, Stat3, C/EBPβ, C/EBPδ, RunX1, FosL2, bHLH-B2, or ZNF238. MGES proteins can be polypeptides encoded by a Stat3, C/EBPβ, C/EBPδ, RunX1, FosL2, bHLH-B2, or ZNF238 nucleotide sequence.

The polypeptide sequence of human signal transducer and activator of transcription 3 (STAT3) is depicted in SEQ ID NO: 231. The nucleotide sequence of human STAT3 is shown in SEQ ID NO: 232. Sequence information related to STAT3 is accessible in public databases by GenBank Accession numbers NM_{—}139276 (for mRNA) and NP_{—}644805 (for protein).

SEQ ID NO: 231 is the human wild type amino acid sequence corresponding to STAT3 (residues 1-769), wherein the bolded sequence represents the mature peptide sequence:

| | |
|---|---|
| 1 | MAQWNQLQQL DTRYLEQLHQ LYSDSFPMEL RQFLAPWIES QDWAYAASKE SHATLVFHNL |
| 61 | LGEIDQQYSR FLQESNVLYQ HNLRRIKQFL QSRYLEKPME IARIVARCLW EESRLLQTAA |
| 121 | TAAQQGGQAN HPTAAVVTEK QQMLEQHLQD VRKRVQDLEQ KMKVVENLQD DFDFNYKTLK |
| 181 | SQGDMQDLNG NNQSVTRQKM QQLEQMLTAL DQMRRSIVSE LAGLLSAMEY VQKTLTDEEL |
| 241 | ADWKRRQQIA CIGGPPNICL DRLENWITSL AESQLQTRQQ IKKLEELQQK VSYKGDPIVQ |
| 301 | HRPMLEERIV ELFRNLMKSA FVVERQPCMP MHPDRPLVIK TGVQFTTKVR LLVKFPELNY |
| 361 | QLKIKVCIDK DSGDVAALRG SRKFNILGTN TKVMNMEESN NGSLSAEFKH LTLREQRCGN |
| 421 | GGRANCDASL IVTEELHLIT FETEVYHQGL KIDLETHSLP VVVISNICQM PNAWASILWY |
| 481 | NMLTNNPKNV NFFTKPPIGT WDQVAEVLSW QFSSTTKRGL SIEQLTTLAE KLLGPGVNYS |
| 541 | GCQITWAKFC KENMAGKGFS FWVWLDNIID LVKKYILALW NEGYIMGFIS KERERAILST |
| 601 | KPPGTFLLRF SESSKEGGVT FTWVEKDISG KTQIQSVEPY TKQQLNNMSF AEIIMGYKIM |
| 661 | DATNILVSPL VYLYPDIPKE EAFGKYCRPE SQEHPEADPG AAPYLKTKFI CVTPTTCSNT |
| 721 | IDLPMSPRTL DSLMQFGNNG EGAEPSAGGQ FESLTFDMEL TSECATSPM |

SEQ ID NO: 232 is the human wild type nucleotide sequence corresponding to STAT3 (nucleotides 1-4978), wherein the underscored bolded “ATG” denotes the beginning of the open reading frame:

| | |
|---|---|
| 1 | ggtttccgga gctgcggcgg cgcagactgg gagggggagc cgggggttcc gacgtcgcag |
| 61 | ccgagggaac aagccccaac cggatcctgg acaggcaccc cggcttggcg ctgtctctcc |
| 121 | ccctcggctc ggagaggccc ttcggcctga gggagcctcg ccgcccgtcc ccggcacacg |
| 181 | cgcagccccg gcctctcggc ctctgccgga gaaacagttg ggacccctga ttttagcagg |
| 241 | atggcccaat ggaatcagct acagcagctt gacacacggt acctggagca gctccatcag |
| 301 | ctctacagtg acagcttccc aatggagctg cggcagtttc tggccccttg gattgagagt |
| 361 | caagattggg catatgcggc cagcaaagaa tcacatgcca ctttggtgtt tcataatctc |
| 421 | ctgggagaga ttgaccagca gtatagccgc ttcctgcaag agtcgaatgt tctctatcag |
| 481 | cacaatctac gaagaatcaa gcagtttctt cagagcaggt atcttgagaa gccaatggag |
| 541 | attgcccgga ttgtggcccg gtgcctgtgg gaagaatcac gccttctaca gactgcagcc |
| 601 | actgcggccc agcaaggggg ccaggccaac caccccacag cagccgtggt gacggagaag |
| 661 | cagcagatgc tggagcagca ccttcaggat gtccggaaga gagtgcagga tctagaacag |
| 721 | aaaatgaaag tggtagagaa tctccaggat gactttgatt tcaactataa aaccctcaag |
| 781 | agtcaaggag acatgcaaga tctgaatgga aacaaccagt cagtgaccag gcagaagatg |
| 841 | cagcagctgg aacagatgct cactgcgctg gaccagatgc ggagaagcat cgtgagtgag |
| 901 | ctggcggggc ttttgtcagc gatggagtac gtgcagaaaa ctctcacgga cgaggagctg |
| 961 | gctgactgga agaggcggca acagattgcc tgcattggag gcccgcccaa catctgccta |
| 1021 | gatcggctag aaaactggat aacgtcatta gcagaatctc aacttcagac ccgtcaacaa |
| 1081 | attaagaaac tggaggagtt gcagcaaaaa gtttcctaca aaggggaccc cattgtacag |
| 1141 | caccggccga tgctggagga gagaatcgtg gagctgttta gaaacttaat gaaaagtgcc |
| 1201 | tttgtggtgg agcggcagcc ctgcatgccc atgcatcctg accggcccct cgtcatcaag |
| 1261 | accggcgtcc agttcactac taaagtcagg ttgctggtca aattccctga gttgaattat |
| 1321 | cagcttaaaa ttaaagtgtg cattgacaaa gactctgggg acgttgcagc tctcagagga |
| 1381 | tcccggaaat ttaacattct gggcacaaac acaaaagtga tgaacatgga agaatccaac |
| 1441 | aacggcagcc tctctgcaga attcaaacac ttgaccctga gggagcagag atgtgggaat |
| 1501 | gggggccgag ccaattgtga tgcttccctg attgtgactg aggagctgca cctgatcacc |
| 1561 | tttgagaccg aggtgtatca ccaaggcctc aagattgacc tagagaccca ctccttgcca |
| 1621 | gttgtggtga tctccaacat ctgtcagatg ccaaatgcct gggcgtccat cctgtggtac |
| 1681 | aacatgctga ccaacaatcc caagaatgta aactttttta ccaagccccc aattggaacc |
| 1741 | tgggatcaag tggccgaggt cctgagctgg cagttctcct ccaccaccaa gcgaggactg |
| 1801 | agcatcgagc agctgactac actggcagag aaactcttgg gacctggtgt gaattattca |
| 1861 | gggtgtcaga tcacatgggc taaattttgc aaagaaaaca tggctggcaa gggcttctcc |
| 1921 | ttctgggtct ggctggacaa tatcattgac cttgtgaaaa agtacatcct ggccctttgg |
| 1981 | aacgaagggt acatcatggg ctttatcagt aaggagcggg agcgggccat cttgagcact |
| 2041 | aagcctccag gcaccttcct gctaagattc agtgaaagca gcaaagaagg aggcgtcact |
| 2101 | ttcacttggg tggagaagga catcagcggt aagacccaga tccagtccgt ggaaccatac |
| 2161 | acaaagcagc agctgaacaa catgtcattt gctgaaatca tcatgggcta taagatcatg |
| 2221 | gatgctacca atatcctggt gtctccactg gtctatctct atcctgacat tcccaaggag |
| 2281 | gaggcattcg gaaagtattg tcggccagag agccaggagc atcctgaagc tgacccaggt |
| 2341 | agcgctgccc catacctgaa gaccaagttt atctgtgtga caccaacgac ctgcagcaat |
| 2401 | accattgacc tgccgatgtc cccccgcact ttagattcat tgatgcagtt tggaaataat |
| 2461 | ggtgaaggtg ctgaaccctc agcaggaggg cagtttgagt ccctcacctt tgacatggag |
| 2521 | ttgacctcgg agtgcgctac ctcccccatg tgaggagctg agaacggaag ctgcagaaag |
| 2581 | atacgactga ggcgcctacc tgcattctgc cacccctcac acagccaaac cccagatcat |
| 2641 | ctgaaactac taactttgtg gttccagatt ttttttaatc tcctacttct gctatctttg |
| 2701 | agcaatctgg gcacttttaa aaatagagaa atgagtgaat gtgggtgatc tgcttttatc |
| 2761 | taaatgcaaa taaggatgtg ttctctgaga cccatgatca ggggatgtgg cggggggtgg |
| 2821 | ctagagggag aaaaaggaaa tgtcttgtgt tgttttgttc ccctgccctc ctttctcagc |
| 2881 | agctttttgt tattgttgtt gttgttctta gacaagtgcc tcctggtgcc tgcggcatcc |
| 2941 | ttctgcctgt ttctgtaagc aaatgccaca ggccacctat agctacatac tcctggcatt |
| 3001 | gcacttttta accttgctga catccaaata gaagatagga ctatctaagc cctaggtttc |
| 3061 | tttttaaatt aagaaataat aacaattaaa gggcaaaaaa cactgtatca gcatagcctt |
| 3121 | tctgtattta agaaacttaa gcagccgggc atggtggctc acgcctgtaa tcccagcact |
| 3181 | ttgggaggcc gaggcggatc ataaggtcag gagatcaaga ccatcctggc taacacggtg |
| 3241 | aaaccccgtc tctactaaaa gtacaaaaaa ttagctgggt gtggtggtgg gcgcctgtag |
| 3301 | tcccagctac tcgggaggct gaggcaggag aatcgcttga acctgagagg cggaggttgc |
| 3361 | agtgagccaa aattgcacca ctgcacactg cactccatcc tgggcgacag tctgagactc |
| 3421 | tgtctcaaaa aaaaaaaaaa aaaaaagaaa cttcagttaa cagcctcctt ggtgctttaa |
| 3481 | gcattcagct tccttcaggc tggtaattta tataatccct gaaacgggct tcaggtcaaa |
| 3541 | cccttaagac atctgaagct gcaacctggc ctttggtgtt gaaataggaa ggtttaagga |
| 3601 | gaatctaagc attttagact tttttttata aatagactta ttttcctttg taatgtattg |
| 3661 | gccttttagt gagtaaggct gggcagaggg tgcttacaac cttgactccc tttctccctg |
| 3721 | gacttgatct gctgtttcag aggctaggtt gtttctgtgg gtgccttatc agggctggga |
| 3781 | tacttctgat tctggcttcc ttcctgcccc accctcccga ccccagtccc cctgatcctg |
| 3841 | ctagaggcat gtctccttgc gtgtctaaag gtccctcatc ctgtttgttt taggaatcct |
| 3901 | ggtctcagga cctcatggaa gaagaggggg agagagttac aggttggaca tgatgcacac |
| 3961 | tatggggccc cagcgacgtg tctggttgag ctcagggaat atggttctta gccagtttct |
| 4021 | tggtgatatc cagtggcact tgtaatggcg tcttcattca gttcatgcag ggcaaaggct |
| 4081 | tactgataaa cttgagtctg ccctcgtatg agggtgtata cctggcctcc ctctgaggct |
| 4141 | ggtgactcct ccctgctggg gccccacagg tgaggcagaa cagctagagg gcctccccgc |
| 4201 | ctgcccgcct tggctggcta gctcgcctct cctgtgcgta tgggaacacc tagcacgtgc |
| 4261 | tggatgggct gcctctgact cagaggcatg gccggatttg gcaactcaaa accaccttgc |
| 4321 | ctcagctgat cagagtttct gtggaattct gtttgttaaa tcaaattagc tggtctctga |
| 4381 | attaaggggg agacgacctt ctctaagatg aacagggttc gccccagtcc tcctgcctgg |
| 4441 | agacagttga tgtgtcatgc agagctctta cttctccagc aacactcttc agtacataat |
| 4501 | aagcttaact gataaacaga atatttagaa aggtgagact tgggcttacc attgggttta |
| 4561 | aatcataggg acctagggcg agggttcagg gcttctctgg agcagatatt gtcaagttca |
| 4621 | tggccttagg tagcatgtat ctggtcttaa ctctgattgt agcaaaagtt ctgagaggag |
| 4681 | ctgagccctg ttgtggccca ttaaagaaca gggtcctcag gccctgcccg cttcctgtcc |
| 4741 | actgccccct ccccatcccc agcccagccg agggaatccc gtgggttgct tacctaccta |
| 4801 | taaggtggtt tataagctgc tgtcctggcc actgcattca aattccaatg tgtacttcat |
| 4861 | agtgtaaaaa tttatattat tgtgaggttt tttgtctttt tttttttttt ttttttttgg |
| 4921 | tatattgctg tatctacttt aacttccaga aataaacgtt atataggaac cgtaaaaa |

The polypeptide sequence of human CCAAT/enhancer binding protein (C/EBP), beta (CEBPB; CEBPβ) is depicted in SEQ ID NO: 233. The nucleotide sequence of human CEBPβ is shown in SEQ ID NO: 234. Sequence information related to CEBPβ is accessible in public databases by GenBank Accession numbers NM_{—}005194 (for mRNA) and NP_{—}005185 (for protein).

SEQ ID NO: 233 is the human wild type amino acid sequence corresponding to CEBPβ (residues 1-345), wherein the bolded sequence represents the mature peptide sequence:

| | |
|---|---|
| 1 | MQRLVAWDPA CLPLPPPPPA FKSMEVANFY YEADCLAAAY GGKAAPAAPP AARPGPRPPA |
| 61 | GELGSIGDHE RAIDFSPYLE PLGAPQAPAP ATATDTFEAA PPAPAPAPAS SGQHHDFLSD |
| 121 | LFSDDYGGKN CKKPAEYGYV SLGRLGAAKG ALHPGCFAPL HPPPPPPPPP AELKAEPGFE |
| 181 | PADCKRKEEA GAPGGGAGMA AGFPYALRAY LGYQAVPSGS SGSLSTSSSS SPPGTPSPAD |
| 241 | AKAPPTACYA GAAPAPSQVK SKAKKTVDKH SDEYKIRRER NNIAVRKSRD KAKMRNLETQ |
| 301 | HKVLELTAEN ERLQKKVEQL SRELSTLRNL FKQLPEPLLA SSGHC |

SEQ ID NO: 234 is the human wild type nucleotide sequence corresponding to CEBPβ (nucleotides 1-1837), wherein the underscored bolded “ATG” denotes the beginning of the open reading frame:

| | |
|---|---|
| 1 | gagccgcgca cgggactggg aaggggaccc acccgagggt ccagccacca gccccctcac |
| 61 | taatagcggc caccccggca gcggcggcag cagcagcagc gacgcagcgg cgacagctca |
| 121 | gagcagggag gccgcgccac ctgcgggccg gccggagcgg gcagccccag gccccctccc |
| 181 | cgggcacccg cgttcatgca acgcctggtg gcctgggacc cagcatgtct ccccctgccg |
| 241 | ccgccgccgc ctgcctttaa atccatggaa gtggccaact tctactacga ggcggactgc |
| 301 | ttggctgctg cgtacggcgg caaggcggcc cccgcggcgc cccccgcggc cagacccggg |
| 361 | ccgcgccccc ccgccggcga gctgggcagc atcggcgacc acgagcgcgc catcgacttc |
| 421 | agcccgtacc tggagccgct gggcgcgccg caggccccgg cgcccgccac ggccacggac |
| 481 | accttcgagg cggctccgcc cgcgcccgcc cccgcgcccg cctcctccgg gcagcaccac |
| 541 | gacttcctct ccgacctctt ctccgacgac tacgggggca agaactgcaa gaagccggcc |
| 601 | gagtacggct acgtgagcct ggggcgcctg ggggccgcca agggcgcgct gcaccccggc |
| 661 | tgcttcgcgc ccctgcaccc accgcccccg ccgccgccgc cgcccgccga gctcaaggcg |
| 721 | gagccgggct tcgagcccgc ggactgcaag cggaaggagg aggccggggc gccgggcggc |
| 781 | ggcgcaggca tggcggcggg cttcccgtac gcgctgcgcg cttacctcgg ctaccaggcg |
| 841 | gtgccgagcg gcagcagcgg gagcctctcc acgtcctcct cgtccagccc gcccggcacg |
| 901 | ccgagccccg ctgacgccaa ggcgcccccg accgcctgct acgcgggggc cgcgccggcg |
| 961 | ccctcgcagg tcaagagcaa ggccaagaag accgtggaca agcacagcga cgagtacaag |
| 1021 | atccggcgcg agcgcaacaa catcgccgtg cgcaagagcc gcgacaaggc caagatgcgc |
| 1081 | aacctggaga cgcagcacaa ggtcctggag ctcacggccg agaacgagcg gctgcagaag |
| 1141 | aaggtggagc agctgtcgcg cgagctcagc accctgcgga acttgttcaa gcagctgccc |
| 1201 | gagcccctgc tcgcctcctc cggccactgc tagcgcggcc cccgcgcgcg tccccctgcc |
| 1261 | ggccggggct gagactccgg ggagcgcccg cgcccgcgcc ctcgcccccg cccccggcgg |
| 1321 | cgccggcaaa actttggcac tggggcactt ggcagcgcgg ggagcccgtc ggtaatttta |
| 1381 | atattttatt atatatatat atctatattt ttgtccaaac caaccgcaca tgcagatggg |
| 1441 | gctcccgccc gtggtgttat ttaaagaaga aacgtctatg tgtacagatg aatgataaac |
| 1501 | tctctgcttc tccctctgcc cctctccagg cgccggcggg cgggccggtt tcgaagttga |
| 1561 | tgcaatcggt ttaaacatgg ctgaacgcgt gtgtacacgg gactgacgca acccacgtgt |
| 1621 | aactgtcagc cgggccctga gtaatcgctt aaagatgttc ctacgggctt gttgctgttg |
| 1681 | atgttttgtt ttgttttgtt ttttggtctt tttttgtatt ataaaaaata atctatttct |
| 1741 | atgagaaaag aggcgtctgt atattttggg aatcttttcc gtttcaagca ttaagaacac |
| 1801 | ttttaataaa cttttttttg agaatggtta caaagcc |

The polypeptide sequence of human CCAAT/enhancer binding protein (C/EBP), delta (CEBPD; CEBPδ) is depicted in SEQ ID NO: 235. The nucleotide sequence of human CEBPδ is shown in SEQ ID NO: 236. Sequence information related to CEBPδ is accessible in public databases by GenBank Accession numbers NM_{—}005195 (for mRNA) and NP_{—}005186 (for protein).

SEQ ID NO: 235 is the human wild type amino acid sequence corresponding to CEBPδ (residues 1-269), wherein the bolded sequence represents the mature peptide sequence:

| | |
|---|---|
| 1 | MSAALFSLDG PARGAPWPAE PAPFYEPGRA GKPGRGAEPG ALGEPGAAAP AMYDDESAID |
| 61 | FSAYIDSMAA VPTLELCHDE LFADLFNSNH KAGGAGPLEL LPGGPARPLG PGPAAPRLLK |
| 121 | REPDWGDGDA PGSLLPAQVA ACAQTVVSLA AAGQPTPPTS PEPPRSSPRQ TPAPGPAREK |
| 181 | SAGKRGPDRG SPEYRQRRER NNIAVRKSRD KAKRRNQEMQ QKLVELSAEN EKLHQRVEQL |
| 241 | TRDLAGLRQF FKQLPSPPFL PAAGTADCR |

SEQ ID NO: 236 is the human wild type nucleotide sequence corresponding to CEBPδ (nucleotides 1-1269), wherein the underscored bolded “ATG” denotes the beginning of the open reading frame:

| | |
|---|---|
| 1 | aggtgacagc ctcgcttgga cgcagagccc ggcccgacgc cgccatgagc gccgcgctct |
| 61 | tcagcctgga cggcccggcg cgcggcgcgc cctggcctgc ggagcctgcg cccttctacg |
| 121 | aaccgggccg ggcgggcaag ccgggccgcg gggccgagcc aggggcccta ggcgagccag |
| 181 | gcgccgccgc ccccgccatg tacgacgacg agagcgccat cgacttcagc gcctacatcg |
| 241 | actccatggc cgccgtgccc accctggagc tgtgccacga cgagctcttc gccgacctct |
| 301 | tcaacagcaa tcacaaggcg ggcggcgcgg ggcccctgga gcttcttccc ggcggccccg |
| 361 | cgcgcccctt gggcccgggc cctgccgctc cccgcctgct caagcgcgag cccgactggg |
| 421 | gcgacggcga cgcgcccggc tcgctgttgc ccgcgcaggt ggccgcgtgc gcacagaccg |
| 481 | tggtgagctt ggcggccgca gggcagccca ccccgcccac gtcgccggag ccgccgcgca |
| 541 | gcagccccag gcagaccccc gcgcccggcc ccgcccggga gaagagcgcc ggcaagaggg |
| 601 | gcccggaccg cggcagcccc gagtaccggc agcggcgcga gcgcaacaac atcgccgtgc |
| 661 | gcaagagccg cgacaaggcc aagcggcgca accaggagat gcagcagaag ttggtggagc |
| 721 | tgtcggctga gaacgagaag ctgcaccagc gcgtggagca gctcacgcgg gacctggccg |
| 781 | gcctccggca gttcttcaag cagctgccca gcccgccctt cctgccggcc gccgggacag |
| 841 | cagactgccg gtaacgcgcg gccggggcgg gagagactca gcaacgaccc atacctcaga |
| 901 | cccgacggcc cggagcggag cgcgccctgc cctggcgcag ccagagccgc cgggtgcccg |
| 961 | ctgcagtttc ttgggacata ggagcgcaaa gaagctacag cctggactta ccaccactaa |
| 1021 | actgcgagag aagctaaacg tgtttatttt cccttaaatt atttttgtaa tggtagcttt |
| 1081 | ttctacatct tactcctgtt gatgcagcta aggtacattt gtaaaaagaa aaaaaaccag |
| 1141 | acttttcaga caaacccttt gtattgtaga taagaggaaa agactgagca tgctcacttt |
| 1201 | tttatattaa tttttacagt atttgtaaga ataaagcagc atttgaaatc gaaaaaaaaa |
| 1261 | aaaaaaaaa |

The polypeptide sequence of human runt-related transcription factor 1 isoform AML1b (RunX1) is depicted in SEQ ID NO: 237. The nucleotide sequence of human RunX1 is shown in SEQ ID NO: 238. Sequence information related to RunX1 is accessible in public databases by GenBank Accession numbers NM_{—}001001890 (for mRNA) and NP_{—}001001890 (for protein).

SEQ ID NO: 237 is the human wild type amino acid sequence corresponding to RunX1 (residues 1-453), wherein the bolded sequence represents the mature peptide sequence:

| | |
|---|---|
| 1 | MRIPVDASTS RRFTPPSTAL SPGKMSEALP LGAPDAGAAL AGKLRSGDRS MVEVLADHPG |
| 61 | ELVRTDSPNF LCSVLPTHWR CNKTLPIAFK VVALGDVPDG TLVTVMAGND ENYSAELRNA |
| 121 | TAAMKNQVAR FNDLRFVGRS GRGKSFTLTI TVFTNPPQVA TYHRAIKITV DGPREPRRHR |
| 181 | QKLDDQTKPG SLSFSERLSE LEQLRRTAMR VSPHHPAPTP NPRASLNHST AFNPQPQSQM |
| 241 | QDTRQIQPSP PWSYDQSYQY LGSIASPSVH PATPISPGRA SGMTTLSAEL SSRLSTAPDL |
| 301 | TAFSDPRQFP ALPSISDPRM HYPGAFTYSP TPVTSGIGIG MSAMGSATRY HTYLPPPYPG |
| 361 | SSQAQGGPFQ ASSPSYHLYY GASAGSYQFS MVGGERSPPR ILPPCTNAST GSALLNPSLP |
| 421 | NQSDVVEAEG SHSNSPTNMA PSARLEEAVW RPY |

SEQ ID NO: 238 is the human wild type nucleotide sequence corresponding to RunX1 (nucleotides 1-7274), wherein the underscored bolded “ATG” denotes the beginning of the open reading frame:

| | |
|---|---|
| 1 | catagagcca gcgggcgcgg gcgggacggg cgccccgcgg ccggacccag ccagggcacc |
| 61 | acgctgcccg gccctgcgcc gccaggcact tctttccggg gctcctaggg acgccagaag |
| 121 | gaagtcaacc tctgctgctt ctccttggcc tgcgttggac cttccttttt ttgttgtttt |
| 181 | tttttgtttt tcccctttct tccttttgaa ttaactggct tcttggctgg atgttttcaa |
| 241 | cttctttcct ggctgcgaac ttttccccaa ttgttttcct tttacaacag ggggagaaag |
| 301 | tgctctgtgg tccgaggcga gccgtgaagt tgcgtgtgcg tggcagtgtg cgtggcagga |
| 361 | tgtgcgtgcg tgtgtaaccc gagccgcccg atctgtttcg atctgcgccg cggagccctc |
| 421 | cctcaaggcc cgctccacct gctgcggtta cgcggcgctc gtgggtgttc gtgcctcgga |
| 481 | gcagctaacc ggcgggtgct gggcgacggt ggaggagtat cgtctcgctg ctgcccgagt |
| 541 | cagggctgag tcacccagct gatgtagaca gtggctgcct tccgaagagt gcgtgtttgc |
| 601 | atgtgtgtga ctctgcggct gctcaactcc caacaaacca gaggaccagc cacaaactta |
| 661 | accaacatcc ccaaacccga gttcacagat gtgggagagc tgtagaaccc tgagtgtcat |
| 721 | cgactgggcc ttcttatgat tgttgtttta agattagctg aagatctctg aaacgctgaa |
| 781 | ttttctgcac tgagcgtttt gacagaattc attgagagaa cagagaacat gacaagtact |
| 841 | tctagctcag cactgctcca actactgaag ctgattttca aggctactta aaaaaatctg |
| 901 | cagcgtacat taatggattt ctgttgtgtt taaattctcc acagattgta ttgtaaatat |
| 961 | tttatgaagt agagcatatg tatatattta tatatacgtg cacatacatt agtagcacta |
| 1021 | cctttggaag tctcagctct tgcttttcgg gactgaagcc agttttgcat gataaaagtg |
| 1081 | gccttgttac gggagataat tgtgttctgt tgggacttta gacaaaactc acctgcaaaa |
| 1141 | aactgacagg cattaactac tggaacttcc aaataatgtg tttgctgatc gttttactct |
| 1201 | tcgcataaat attttaggaa gtgtatgaga attttgcctt caggaacttt tctaacagcc |
| 1261 | aaagacagaa cttaacctct gcaagcaaga ttcgtggaag atagtctcca ctttttaatg |
| 1321 | cactaagcaa tcggttgcta ggagcccatc ctgggtcaga ggccgatccg cagaaccaga |
| 1381 | acgttttccc ctcctggact gttagtaact tagtctccct cctcccctaa ccacccccgc |
| 1441 | ccccccccac cccccgcagt aataaaggcc cctgaacgtg tatgttggtc tcccgggagc |
| 1501 | tgcttgctga agatccgcgc ccctgtcgcc gtctggtagg agctgtttgc agggtcctaa |
| 1561 | ctcaatcggc ttgttgtgat gcgtatcccc gtagatgcca gcacgagccg ccgcttcacg |
| 1621 | ccgccttcca ccgcgctgag cccaggcaag atgagcgagg cgttgccgct gggcgccccg |
| 1681 | gacgccggcg ctgccctggc cggcaagctg aggagcggcg accgcagcat ggtggaggtg |
| 1741 | ctggccgacc acccgggcga gctggtgcgc accgacagcc ccaacttcct ctgctccgtg |
| 1801 | ctgcctacgc actggcgctg caacaagacc ctgcccatcg ctttcaaggt ggtggcccta |
| 1861 | ggggatgttc cagatggcac tctggtcact gtgatggctg gcaatgatga aaactactcg |
| 1921 | gctgagctga gaaatgctac cgcagccatg aagaaccagg ttgcaagatt taatgacctc |
| 1981 | aggtttgtcg gtcgaagtgg aagagggaaa agcttcactc tgaccatcac tgtcttcaca |
| 2041 | aacccaccgc aagtcgccac ctaccacaga gccatcaaaa tcacagtgga tgggccccga |
| 2101 | gaacctcgaa gacatcggca gaaactagat gatcagacca agcccgggag cttgtccttt |
| 2161 | tccgagcggc tcagtgaact ggagcagctg cggcgcacag ccatgagggt cagcccacac |
| 2221 | cacccagccc ccacgcccaa ccctcgtgcc tccctgaacc actccactgc ctttaaccct |
| 2281 | cagcctcaga gtcagatgca ggatacaagg cagatccaac catccccacc gtggtcctac |
| 2341 | gatcagtcct accaatacct gggatccatt gcctctcctt ctgtgcaccc agcaacgccc |
| 2401 | atttcacctg gacgtgccag cggcatgaca accctctctg cagaactttc cagtcgactc |
| 2461 | tcaacggcac ccgacctgac agcgttcagc gacccgcgcc agttccccgc gctgccctcc |
| 2521 | atctccgacc cccgcatgca ctatccaggc gccttcacct actccccgac gccggtcacc |
| 2581 | tcgggcatcg gcatcggcat gtcggccatg ggctcggcca cgcgctacca cacctacctg |
| 2641 | ccgccgccct accccggctc gtcgcaagcg cagggaggcc cgttccaagc cagctcgccc |
| 2701 | tcctaccacc tgtactacgg cgcctcggcc ggctcctacc agttctccat ggtgggcggc |
| 2761 | gagcgctcgc cgccgcgcat cctgccgccc tgcaccaacg cctccaccgg ctccgcgctg |
| 2821 | ctcaacccca gcctcccgaa ccagagcgac gtggtggagg ccgagggcag ccacagcaac |
| 2881 | tcccccacca acatggcgcc ctccgcgcgc ctggaggagg ccgtgtggag gccctactga |
| 2941 | ggcgccaggc ctggcccggc tgggccccgc gggccgccgc cttcgcctcc gggcgcgcgg |
| 3001 | gcctcctgtt cgcgacaagc ccgccgggat cccgggccct gggcccggcc accgtcctgg |
| 3061 | ggccgagggc gcccgacggc caggatctcg ctgtaggtca ggcccgcgca gcctcctgcg |
| 3121 | cccagaagcc cacgccgccg ccgtctgctg gcgccccggc cctcgcggag gtgtccgagg |
| 3181 | cgacgcacct cgagggtgtc cgccggcccc agcacccagg ggacgcgctg gaaagcaaac |
| 3241 | aggaagattc ccggagggaa actgtgaatg cttctgattt agcaatgctg tgaataaaaa |
| 3301 | gaaagatttt atacccttga cttaactttt taaccaagtt gtttattcca aagagtgtgg |
| 3361 | aattttggtt ggggtggggg gagaggaggg atgcaactcg ccctgtttgg catctaattc |
| 3421 | ttatttttaa tttttccgca ccttatcaat tgcaaaatgc gtatttgcat ttgggtggtt |
| 3481 | tttattttta tatacgttta tataaatata tataaattga gcttgcttct ttcttgcttt |
| 3541 | gaccatggaa agaaatatga ttcccttttc tttaagtttt atttaacttt tcttttggac |
| 3601 | ttttgggtag ttgttttttt ttgttttgtt ttgttttttt gagaaacagc tacagctttg |
| 3661 | ggtcattttt aactactgta ttcccacaag gaatccccag atatttatgt atcttgatgt |
| 3721 | tcagacattt atgtgttgat aattttttaa ttatttaaat gtacttatat taagaaaaat |
| 3781 | atcaagtact acattttctt ttgttcttga tagtagccaa agttaaatgt atcacattga |
| 3841 | agaaggctag aaaaaaagaa tgagtaatgt gatcgcttgg ttatccagaa gtattgttta |
| 3901 | cattaaactc cctttcatgt taatcaaaca agtgagtagc tcacgcagca acgtttttaa |
| 3961 | taggattttt agacactgag ggtcactcca aggatcagaa gtatggaatt ttctgccagg |
| 4021 | ctcaacaagg gtctcatatc taacttcctc cttaaaacag agaaggtcaa tctagttcca |
| 4081 | gagggttgag gcaggtgcca ataattacat ctttggagag gatttgattt ctgcccaggg |
| 4141 | atttgctcac cccaaggtca tctgataatt tcacagatgc tgtgtaacag aacacagcca |
| 4201 | aagtaaactg tgtaggggag ccacatttac ataggaacca aatcaatgaa tttaggggtt |
| 4261 | acgattatag caatttaagg gcccaccaga agcaggcctc gaggagtcaa tttgcctctg |
| 4321 | tgtgcctcag tggagacaag tgggaaaaca tggtcccacc tgtgcgagac cccctgtcct |
| 4381 | gtgctgctca ctcaacaaca tctttgtgtt gctttcacca ggctgagacc ctaccctatg |
| 4441 | gggtatatgg gcttttacct gtgcaccagt gtgacaggaa agattcatgt cactactgtc |
| 4501 | cgtggctaca attcaaaggt atccaatgtc gctgtaaatt ttatggcact atttttattg |
| 4561 | gaggatttgg tcagaatgca gttgttgtac aactcataaa tactaactgc tgattttgac |
| 4621 | acatgtgtgc tccaaatgat ctggtggtta tttaacgtac ctcttaaaat tcgttgaaac |
| 4681 | gatttcaggt caactctgaa gagtatttga aagcaggact tcagaacagt gtttgatttt |
| 4741 | tattttataa atttaagcat tcaaattagg caaatctttg gctgcaggca gcaaaaacag |
| 4801 | ctggacttat ttaaaacaac ttgtttttga gttttcttat atatatattg attatttgtt |
| 4861 | ttacacacat gcagtagcac tttggtaaga gttaaagagt aaagcagctt atgttgtcag |
| 4921 | gtcgttctta tctagagaag agctatagca gatctcggac aaactcagaa tatattcact |
| 4981 | ttcatttttg acaggattcc ctccacaact cagtttcata tattattccg tattacattt |
| 5041 | ttgcagctaa attaccataa aatgtcagca aatgtaaaaa tttaatttct gaaaagcacc |
| 5101 | attagcccat ttcccccaaa ttaaacgtaa atgttttttt tcagcacatg ttaccatgtc |
| 5161 | tgacctgcaa aaatgctgga gaaaaatgaa ggaaaaaatt atgtttttca gtttaattct |
| 5221 | gttaactgaa gatattccaa ctcaaaacca gcctcatgct ctgattagat aatcttttac |
| 5281 | attgaacctt tactctcaaa gccatgtgtg gagggggctt gtcactattg taggctcact |
| 5341 | ggattggtca tttagagttt cacagactct taccagcata tatagtattt aattgtttca |
| 5401 | aaaaaaatca aactgtagtt gttttggcga taggtctcac gcaacacatt tttgtatgtg |
| 5461 | tgtgtgtgtg cgtgtgtgtg tgtgtgtgtg aaaaattgca ttcattgact tcaggtagat |
| 5521 | taaggtatct ttttattcat tgccctcagg aaagttaagg tatcaatgag acccttaagc |
| 5581 | caatcatgta ataactgcat gtgtctggtc caggagaagt attgaataag ccatttctac |
| 5641 | tgcttactca tgtccctatt tatgatttca acatggatac atatttcagt tctttctttt |
| 5701 | tctcactatc tgaaaataca tttccctccc tctcttcccc ccaatatctc cctttttttc |
| 5761 | tctcttcctc tatcttccaa accccacttt ctccctcctc cttttcctgt gttctcttaa |
| 5821 | gcagatagca cataccccca cccagtacca aatttcagaa cacaagaagg tccagttctt |
| 5881 | cccccttcac ataaaggaac atggtttgtc agcctttctc ctgtttatgg gtttcttcca |
| 5941 | gcagaacaga gacattgcca accatattgg atctgcttgc tgtccaaacc agcaaacttt |
| 6001 | cctgggcaaa tcacaatcag tgagtaaata gacagccttt ctgctgcctt gggtttctgt |
| 6061 | gcagataaac agaaatgctc tgattagaaa ggaaatgaat ggttccactc aaatgtcctg |
| 6121 | caatttagga ttgcagattt ctgccttgaa atacctgttt ctttgggaca ttccgtcctg |
| 6181 | atgattttta tttttgttgg tttttatttt tggggggaat gacatgtttg ggtcttttat |
| 6241 | acatgaaaat ttgtttgaca ataatctcac aaaacatatt ttacatctga acaaaatgcc |
| 6301 | tttttgttta ccgtagcgta tacatttgtt ttgggatttt tgtgtgtttg ttgggaattt |
| 6361 | tgtttttagc caggtcagta ttgatgaggc tgatcatttg gctctttttt tccttccaga |
| 6421 | agagttgcat caacaaagtt aattgtattt atgtatgtaa atagatttta agcttcatta |
| 6481 | taaaatattg ttaatgccta taactttttt tcaatttttt tgtgtgtgtt tctaaggact |
| 6541 | ttttcttagg tttgctaaat actgtaggga aaaaaatgct tctttctact ttgtttattt |
| 6601 | tagactttaa aatgagctac ttcttattca cttttgtaaa cagctaatag catggttcca |
| 6661 | atttttttta agttcacttt ttttgttcta ggggaaatga atgtgcaaaa aaagaaaaag |
| 6721 | aactgttggt tatttgtgtt attctggatg tataaaaatc aatggaaaaa aataaacttt |
| 6781 | caaattgaaa tgacggtata acacatctac tgaaaaagca acgggaaatg tggtcctatt |
| 6841 | taagccagcc cccacctagg gtctatttgt gtggcagtta ttgggtttgg tcacaaaaca |
| 6901 | tcctgaaaat tcgtgcgtgg gcttctttct ccctggtaca aacgtatgga atgcttctta |
| 6961 | aaggggaact gtcaagctgg tgtcttcagc cagatgacat gagagaatat cccagaaccc |
| 7021 | tctctccaag gtgtttctag atagcacagg agagcaggca ctgcactgtc cacagtccac |
| 7081 | ggtacacagt cgggtgggcc gcctcccctc tcctgggagc attcgtcgtg cccagcctga |
| 7141 | gcagggcagc tggactgctg ctgttcagga gccaccagag ccttcctctc tttgtaccac |
| 7201 | agtttcttct gtaaatccag tgttacaatc agtgtgaatggcaaataaaca gtttgacaa |
| 7261 | gtacatacac cata |

The polypeptide sequence of human FOS-like antigen 2 (FOSL2) is depicted in SEQ ID NO: 239. The nucleotide sequence of human FOSL2 is shown in SEQ ID NO: 240. Sequence information related to FOSL2 is accessible in public databases by GenBank Accession numbers NM_{—}005253 (for mRNA) and NP_{—}005244 (for protein).

SEQ ID NO: 239 is the human wild type amino acid sequence corresponding to FOSL2 (residues 1-326), wherein the bolded sequence represents the mature peptide sequence:

| | |
|---|---|
| 1 | MYQDYPGNFD TSSRGSSGSP AHAESYSSGG GGQQKFRVDM PGSGSAFIPT INAITTSQDL |
| 61 | QWMVQPTVIT SMSNPYPRSH PYSPLPGLAS VPGHMALPRP GVIKTIGTTV GRRRRDEQLS |
| 121 | PEEEEKRRIR RERNKLAAAK CRNRRRELTE KLQAETEELE EEKSGLQKEI AELQKEKEKL |
| 181 | EFMLVAHGPV CKISPEERRS PPAPGLQPMR SGGGSVGAVV VKQEPLEEDS PSSSSAGLDK |
| 241 | AQRSVIKPIS IAGGFYGEEP LHTPIVVTST PAVTPGTSNL VFTYPSVLEQ ESPASPSESC |
| 301 | SKAHRRSSSS GDQSSDSLNS PTLLAL |

SEQ ID NO: 240 is the human wild type nucleotide sequence corresponding to FOSL2 (nucleotides 1-4015), wherein the underscored bolded “ATG” denotes the beginning of the open reading frame:

| | |
|---|---|
| 1 | cgaacgagcg gcgctcggcg gggacagaaa gagggagaga gagagagaga gagagggaga |
| 61 | ggcgcggccg ggcgaggcgg gcccgtccgg gagcgggctc cggggaaggg gtgcgggtct |
| 121 | gggcgccgga gcggggagcg gggccgcgtc cctctcagcg ccagctctac ttgagcccca |
| 181 | cgagccgctg tccccctggc gcgctcgggg ccgcgggacg ggcgcacgcc gccttctcct |
| 241 | agtcaagtat ccgagccgcc ccgaaactcg ggcggcgagt cggccacggg aagtttattc |
| 301 | tccggctcct tttctaaaag gaagaaacag aagtttctcc cagcggacag cttttctttc |
| 361 | cgcctttttg gccctgtctg aaatcggggg tccccagggc tggcaggcca ggctcgctgg |
| 421 | gctcctaatc ttttttttaa tttccaattt ttgattgggc cgtgggtccc cgctgagctc |
| 481 | cggctgcgcg cgggggcggg agggcgcgcg caggggaggg accgagagac gcgccgactt |
| 541 | tttagaggga gggatcgggt ggacaactgg tcccgcggcg ctcgcagagc cggaaagaag |
| 601 | tgctgtaagg gacgctcggg ggacgctgtt cctgaggtgt cgccgcctcc ctgtcctcgc |
| 661 | cctccgcggt gggggagaaa cccaggagcg aagcccagag cccgcggcgc ggccggcgga |
| 721 | cgaacgagcg cgcagcagcc ggtgcgcggc cgcggcgagg gcgggggaag aaaaacaccc |
| 781 | tgtttcctct ccggccccca ccgcggatca tgtaccagga ttatcccggg aactttgaca |
| 841 | cctcgtcccg gggcagcagc ggctctcctg cgcacgccga gtcctactcc agcggcggcg |
| 901 | gcggccagca gaaattccgg gtagatatgc ctggctcagg cagtgcattc atccccacca |
| 961 | tcaacgccat cacgaccagc caggacctgc agtggatggt gcagcccaca gtgatcacct |
| 1021 | ccatgtccaa cccataccct cgctcgcacc cctacagccc cctgccgggc ctggcctctg |
| 1081 | tccctggaca catggccctc ccaagacctg gcgtgatcaa gaccattggc accaccgtgg |
| 1141 | gccgcaggag gagagatgag cagctgtctc ctgaagagga ggagaagcgt cgcatccggc |
| 1201 | gggagaggaa caagctggct gcagccaagt gccggaaccg acgccgggag ctgacagaga |
| 1261 | agctgcaggc ggagacagag gagctggagg aggagaagtc aggcctgcag aaggagattg |
| 1321 | ctgagctgca gaaggagaag gagaagctgg agttcatgtt ggtggctcac ggcccagtgt |
| 1381 | gcaagattag ccccgaggag cgccgatcgc ccccagcccc tgggctgcag cccatgcgca |
| 1441 | gtgggggtgg ctcggtgggc gctgtagtgg tgaaacagga gcccctggaa gaggacagcc |
| 1501 | cctcgtcctc gtcggcgggg ctggacaagg cccagcgctc tgtcatcaag cccatcagca |
| 1561 | ttgctggggg cttctacggt gaggagcccc tgcacacccc catcgtggtg acctccacac |
| 1621 | ctgctgtcac tccgggcacc tcgaacctcg tcttcaccta tcctagcgtc ctggagcagg |
| 1681 | agtcacccgc atctccctcc gaatcctgct ccaaggctca ccgcagaagc agtagcagcg |
| 1741 | gggaccaatc atcagactcc ttgaactccc ccactctgct ggctctgtaa cccagtgcac |
| 1801 | ctccctcccc agctccggag ggggtcctcc tcgctcctcc ttcccaggga ccagcacctt |
| 1861 | caagcgctcc agggccgtga gggcaagagg gggacctgcc accagggagc ttcctggctc |
| 1921 | tgggggaccc aggtgggact tagcagtgag tattggaaga cttgggttga tctcttagaa |
| 1981 | gccatgggac ctcctccctc attcatcttg caagcaaatc ccatttcttg aaaagccttg |
| 2041 | gagaactcgg tttggtagac ttggacatct ctctggcttc tgaagagcct gaagctggcc |
| 2101 | tggaccattc ctgtcccttt gttaccatac tgtctctgga gtgatggtgt ccttccctgc |
| 2161 | cccaccacgc atgctcagtg ccttttggtt tcaccttccc tcgacttgac cctttcctcc |
| 2221 | cccagcgtca gtttcactcc ctcttggttt ttatcaaatt tgccatgaca tttcatctgg |
| 2281 | gtggtctgaa tattaaagct cttcatttct ggagatgggg cagcaggtgg ctcttctgct |
| 2341 | ggggctgact tgtccagaag gggacaaagt gcaatacaga gccttcccta ccctgacgcc |
| 2401 | tcccagtcat catctccaga actcccagcg gggctccctg agctctcaag gagatgctgc |
| 2461 | catcactggg aggctcagag gacccttcct gcccaccttc ggagacggct tctggaggaa |
| 2521 | cggcttggcc agaagacagg gtgtgagtga gacagtgggg cacaggttgg gtttgccaaa |
| 2581 | cgcctaatta ccaggccagg aagcatgcca acaaagccac acgggtgtcc tagccagctt |
| 2641 | cccttcacct ggtgtcttga gtagggcgtc tcctgtaatt actgccttgc cattctgccc |
| 2701 | ctggaccctt ctctccggac cagggaggcg tccctcccta ggagccacac attatactcc |
| 2761 | aagtccctgc cgggctccgc ctttccccca ccctggctct cagggtgacg ccaccacag |
| 2821 | agatttaatg agcgtgggcc tggaccttcc ccagatgctg ccaggcagcc cctccccaag |
| 2881 | cctcaaagaa gcatttgctg aggatggaga ggcaggggag ggaggcggga ggccgtcact |
| 2941 | ggagtggcgt ctgcagcagc tgctgcccca gcacccgctc agcctgtcct ggctgctcac |
| 3001 | ctccccgcag ggcaccgggc ctttcctgcc ctctgtggtc atctgccacc tgctggatca |
| 3061 | agtgctttct cttttacact cccctgtccc caccccagtg cactcttctg gcccaggcag |
| 3121 | caagcaagct gtgaacagct ggcctgagct gtcgctgtgg cttgtggctc atgcgccatt |
| 3181 | cctggttgtc tgttgaatct ttctggctgc tggaattgga gataggatgt tttgcttccc |
| 3241 | actgcaggag agctgccccc tttcacgggg ttggggaagg gtccccctgg cctccagcag |
| 3301 | gagcacagct cagcagggtc cctgctgccc acccctctga gccttttctc cccagggtat |
| 3361 | ggctcctgct gagtttcttg tccagcaggg ccttgacagg aatccaggga gtagctcctg |
| 3421 | gccagaacca gcctctgcgg ggcttgtgct ctgcaaagac tctgctgctg gggattcagc |
| 3481 | tctagaggtc acagtatcct cgtttgaaag ataattaaga tcccccgtgg agaaagcagt |
| 3541 | gacacattca cacagctgtt ccctcgcatg ttatttcatg aacatgacct gttttcgtgc |
| 3601 | actagacaca cagagtggaa cagccgtatg cttaaagtac atgggccagt gggactggaa |
| 3661 | gtgacctgta caagtgatgc agaaaggagg gtttcaaaga aaaaggattt tgtttaaaat |
| 3721 | actttaaaaa tgttatttcc tgcatccctt ggctgtgatg cccctctccc gatttcccag |
| 3781 | gggctctggg agggaccctt ctaagaagat tgggcagttg ggtttctggc ttgagatgaa |
| 3841 | tccaagcagc agaatgagcc aggagtagca ggagatgggc aaagaaaact ggggtgcact |
| 3901 | cagctctcac aggggtaatc atctcaagtg gtatttgtag ccaagtggga gctattttct |
| 3961 | tttttgtgca tatagatatt tcttaaatga aaaaaaaaaa aaaaaaaaaa aaaaa |

Class E basic helix-loop-helix protein 40 is a protein that in humans is encoded by the BHLHE40 gene, also referred to as BHLHB2 (bHLH-B2, as used herein). BHLHB2 is depicted in SEQ ID NO: 241. The nucleotide sequence of human BHLHB2 is shown in SEQ ID NO: 242. Sequence information related to BHLHB2 is accessible in public databases by GenBank Accession numbers NM_{—}003670 (for mRNA) and NP_{—}003661 (for protein).

SEQ ID NO: 241 is the human wild type amino acid sequence corresponding to BHLHB2 (residues 1-412), wherein the bolded sequence represents the mature peptide sequence:

| | |
|---|---|
| 1 | MERIPSAQPP PACLPKAPGL EHGDLPGMYP AHMYQVYKSR RGIKRSEDSK ETYKLPHRLI |
| 61 | EKKRRDRINE CIAQLKDLLP EHLKLTTLGH LEKAVVLELT LKHVKALTNL IDQQQQKIIA |
| 121 | LQSGLQAGEL SGRNVETGQE MFCSGFQTCA REVLQYLAKH ENTRDLKSSQ LVTHLHRVVS |
| 181 | ELLQGGTSRK PSDPAPKVMD FKEKPSSPAK GSEGPGKNCV PVIQRTFAHS SGEQSGSDTD |
| 241 | TDSGYGGESE KGDLRSEQPC FKSDHGRRFT MGERIGAIKQ ESEEPPTKKN RMQLSDDEGH |
| 301 | FTSSDLISSP FLGPHPHQPP FCLPFYLIPP SATAYLPMLE KCWYPTSVPV LYPGLNASAA |
| 361 | ALSSFMNPDK ISAPLLMPQR LPSPLPAHPS VDSSVLLQAL KPIPPLNLET KD |

SEQ ID NO: 242 is the human wild type nucleotide sequence corresponding to BHLHB2 (nucleotides 1-3061), wherein the underscored bolded “ATG” denotes the beginning of the open reading frame:

| | |
|---|---|
| 1 | cgcctccccg cccgccccac ttctcattca cttggctcgc acggcgcaga cagaccgcgc |
| 61 | agggagcaca caccgccagt ctgtgcgctg agtcggagcc agaggccgcg gggacaccgg |
| 121 | gccatgcacg cccccaactg aagctgcatc tcaaagccga agattccagc agcccagggg |
| 181 | atttcaaaga gctcagactc agaggaacat ctgcggagag acccccgaag ccctctccag |
| 241 | ggcagtcctc atccagacgc tccgctagtg cagacaggag cgcgcagtgg ccccggctcg |
| 301 | ccgcgccatg gagcggatcc ccagcgcgca accacccccc gcctgcctgc ccaaagcacc |
| 361 | gggactggag cacggagacc taccagggat gtaccctgcc cacatgtacc aagtgtacaa |
| 421 | gtcaagacgg ggaataaagc ggagcgagga cagcaaggag acctacaaat tgccgcaccg |
| 481 | gctcatcgag aaaaagagac gtgaccggat taacgagtgc atcgcccagc tgaaggatct |
| 541 | cctacccgaa catctcaaac ttacaacttt gggtcacttg gaaaaagcag tggttcttga |
| 601 | acttaccttg aagcatgtga aagcactaac aaacctaatt gatcagcagc agcagaaaat |
| 661 | cattgccctg cagagtggtt tacaagctgg tgagctgtca gggagaaatg tcgaaacagg |
| 721 | tcaagagatg ttctgctcag gtttccagac atgtgcccgg gaggtgcttc agtatctggc |
| 781 | caagcacgag aacactcggg acctgaagtc ttcgcagctt gtcacccacc tccaccgggt |
| 841 | ggtctcggag ctgctgcagg gtggtacctc caggaagcca tcagacccag ctcccaaagt |
| 901 | gatggacttc aaggaaaaac ccagctctcc ggccaaaggt tcggaaggtc ctgggaaaaa |
| 961 | ctgcgtgcca gtcatccagc ggactttcgc tcactcgagt ggggagcaga gcggcagcga |
| 1021 | cacggacaca gacagtggct atggaggaga atcggagaag ggcgacttgc gcagtgagca |
| 1081 | gccgtgcttc aaaagtgacc acggacgcag gttcacgatg ggagaaagga tcggcgcaat |
| 1141 | taagcaagag tccgaagaac cccccacaaa aaagaaccgg atgcagcttt cggatgatga |
| 1201 | aggccatttc actagcagtg acctgatcag ctccccgttc ctgggcccac acccacacca |
| 1261 | gcctcctttc tgcctgccct tctacctgat cccaccttca gcgactgcct acctgcccat |
| 1321 | gctggagaag tgctggtatc ccacctcagt gccagtgcta tacccaggcc tcaacgcctc |
| 1381 | tgccgcagcc ctctctagct tcatgaaccc agacaagatc tcggctccct tgctcatgcc |
| 1441 | ccagagactc ccttctccct tgccagctca tccgtccgtc gactcttctg tcttgctcca |
| 1501 | agctctgaag ccaatccccc ctttaaactt agaaaccaaa gactaaactc tctaggggat |
| 1561 | cctgctgctt tgctttcctt cctcgctact tcctaaaaag caacaaaaaa gtttttgtga |
| 1621 | atgctgcaag attgttgcat tgtgtatact gagataatct gaggcatgga gagcagattc |
| 1681 | agggtgtgtg tgtgtgtgtg tgtgtgtgta tgtgcgtgtg cgtgcacatg tgtgcctgcg |
| 1741 | tgttggtata ggactttaaa gctccttttg gcatagggaa gtcacgaagg attgcttgac |
| 1801 | atcaggagac ttggggggga ttgtagcaga cgtctgggct tttccccacc cagagaatag |
| 1861 | cccccttcga tacacatcag ctggattttc aaaagcttca aagtcttggt ctgtgagtca |
| 1921 | ctcttcagtt tgggagctgg gtctgtggct ttgatcagaa ggtactttca aaagagggct |
| 1981 | ttccagggct cagctcccaa ccagctgtta ggaccccacc cttttgcctt tattgtcgac |
| 2041 | gtgactcacc agacgtcggg gagagagagc agtcagaccg agctttctgc taacatgggg |
| 2101 | aggtagcagg cactggcata gcacggtagt ggtttgggga ggtttccgca ggtctgctcc |
| 2161 | ccacccctgc ctcggaagaa taaagagaat gtagttccct actcaggctt tcgtagtgat |
| 2221 | tagcttacta aggaactgaa aatgggcccc ttgtacaagc tgagctgccc cggagggagg |
| 2281 | gaggagttcc ctgggcttct ggcacctgtt tctaggccta accattagta cttactgtgc |
| 2341 | agggaaccaa accaaggtct gagaaatgcg gacaccccga gcgagcaccc caaagtgcac |
| 2401 | aaagctgagt aaaaagctgc ccccttcaaa cagaactaga ctcagttttc aattccatcc |
| 2461 | taaaactcct tttaaccaag cttagcttct caaaggccta accaagcctt ggcaccgcca |
| 2521 | gatcctttct gtaggctaat tcctcttgcc caacggcata tggagtgtcc ttattgctaa |
| 2581 | aaaggattcc gtctccttca aagaagtttt atttttggtc cagagtactt gttttcccga |
| 2641 | tgtgtccagc cagctccgca gcagcttttc aaaatgcact atgcctgatt gctgatcgtg |
| 2701 | ttttaacttt ttcttttcct gtttttattt tggtattaag tcgttgcctt tatttgtaaa |
| 2761 | gctgttataa atatatatta tataaatata ttaaaaagga aaatgtttca gatgtttatt |
| 2821 | tgtataatta cttgattcac acagtgagaa aaaatgaatg tattcctgtt tttgaagaga |
| 2881 | agaataattt tttttttctc tagggagagg tacagtgttt atattttgga gccttcctga |
| 2941 | aggtgtaaaa ttgtaaatat ttttatctat gagtaaatgt taagtagttg ttttaaaata |
| 3001 | cttaataaaa taattctttt cctgtggaag agaaaaaaaa aaaaaaaaaa aaaaaaaaaa |
| 3061 | a |

The polypeptide sequence of human zinc finger protein 238 isoform 2 (ZNF238) is depicted in SEQ ID NO: 243. The nucleotide sequence of human ZNF238 is shown in SEQ ID NO: 244. Sequence information related to ZNF238 is accessible in public databases by GenBank Accession numbers NM_{—}006352 (for mRNA) and NP_{—}006343 (for protein).

SEQ ID NO: 243 is the human wild type amino acid sequence corresponding to ZNF238 (residues 1-522), wherein the bolded sequence represents the mature peptide sequence:

| | |
|---|---|
| 1 | MEFPDHSRHL LQCLSEQRHQ GFLCDCTVLV GDAQFRAHRA VLASCSMYFH LFYKDQLDKR |
| 61 | DIVHLNSDIV TAPAFALLLE FMYEGKLQFK DLPIEDVLAA ASYLHMYDIV KVCKKKLKEK |
| 121 | ATTEADSTKK EEDASSCSDK VESLSDGSSH IAGDLPSDED EGEDEKLNIL PSKRDLAAEP |
| 181 | GNMWMRLPSD SAGIPQAGGE AEPHATAAGK TVASPCSSTE SLSQRSVTSV RDSADVDCVL |
| 241 | DLSVKSSLSG VENLNSSYFS SQDVLRSNLV QVKVEKEASC DESDVGTNDY DMEHSTVKES |
| 301 | VSTNNRVQYE PAHLAPLRED SVLRELDRED KASDDEMMTP ESERVQVEGG MESSLLPYVS |
| 361 | NILSPAGQIF MCPLCNKVFP SPHILQIHLS THFREQDGIR SKPAADVNVP TCSLCGKTFS |
| 421 | CMYTLKRHER THSGEKPYTC TQCGKSFQYS HNLSRHAVVH TREKPHACKW CERRFTQSGD |
| 481 | LYRHIRKFHC ELVNSLSVKS EALSLPTVRD WTLEDSSQEL WK |

SEQ ID NO: 244 is the human wild type nucleotide sequence corresponding to ZNF238 (nucleotides 1-4244), wherein the underscored bolded “ATG” denotes the beginning of the open reading frame:

| | |
|---|---|
| 1 | tttaaactgt gctttctaag cacagtcagg tagcaaaagt aataaaaagg atggttgaac |
| 61 | aagttttctt gtatgttcca ggatatgttt gggacttttc tttgtttatt atatgagttg |
| 121 | ttccctttga aattaaagct attttgtagg ttttgtggga cataatttga taagtagagt |
| 181 | taattaaatt tcttctggaa gagatctaaa ttcttattct tagtgagaga ctgtagttaa |
| 241 | aggaaggctt ttagaacttg ggttcaagga agatggagat gcgtcggaag ctctttggcg |
| 301 | ggggtgagga agttcagaaa gtgtgcattt tccttctggc atttaggtct tgtccgtgtg |
| 361 | atttggtggt gcttgggtca taagcctgat taaaattcag ggacatgtac cacggcggcc |
| 421 | aaagcggaat taattttttt atatggggac tggagcgctg aaaagttgtt cctgaccagg |
| 481 | ctctaatgag aaattcctct ctccccaggt tatgaagaca gtatggagtt tccagaccat |
| 541 | agtagacatt tgctacagtg tctgagcgag cagagacacc agggttttct ttgtgactgc |
| 601 | actgttctgg tgggagatgc ccagttccga gcgcaccgag ctgtactggc ttcatgcagc |
| 661 | atgtatttcc acctctttta caaggaccag ctggacaaaa gagacattgt tcatctgaac |
| 721 | agcgacattg ttacagcccc cgctttcgct ctcctgcttg aattcatgta tgaagggaaa |
| 781 | ctccagttca aagacttgcc cattgaagac gtgctagcag ctgccagtta tctccacatg |
| 841 | tatgacattg tcaaagtctg caaaaagaag ctgaaagaga aagccaccac ggaggcagac |
| 901 | agcaccaaaa aggaagaaga tgcttcaagt tgttcggaca aagtcgagag tctctccgat |
| 961 | ggcagcagcc acatagcagg cgatttgccc agtgatgaag atgaaggaga agatgaaaaa |
| 1021 | ttgaacatcc tgcccagcaa aagggacttg gcggccgagc ctgggaacat gtggatgcga |
| 1081 | ttgccctcag actcagcagg catcccccag gctggcggag aggcagagcc acacgccaca |
| 1141 | gcagctggaa aaacagtagc cagcccctgc agctcaacag agtctttgtc ccagaggtct |
| 1201 | gtcacctccg tgagggattc ggcagatgtt gactgtgtgc tggacctgtc tgtcaagtcc |
| 1261 | agcctttcag gagttgaaaa tctgaacagc tcttatttct cttcacagga cgtgctgaga |
| 1321 | agcaacctgg tgcaggtgaa ggtggagaaa gaggcttcct gtgatgagag tgatgttggc |
| 1381 | actaatgact atgacatgga acatagcact gtgaaagaaa gtgtgagcac taataacagg |
| 1441 | gtacagtatg agccggccca tctggctccc ctgagggagg actcggtctt gagggagctg |
| 1501 | gaccgggagg acaaagccag tgatgatgag atgatgaccc cagagagcga gcgtgtccag |
| 1561 | gtggagggag gcatggagag cagtctgctc ccctacgtct ccaacatcct gagccccgcg |
| 1621 | ggccagatct tcatgtgccc cctgtgcaac aaggtcttcc ccagccccca catcctgcag |
| 1681 | atccacctga gcacgcactt ccgcgagcag gacggcatcc gcagcaagcc cgccgccgat |
| 1741 | gtcaacgtgc ccacgtgctc gctgtgtggg aagactttct cttgcatgta caccctcaag |
| 1801 | cgccacgaga ggactcactc gggggagaag ccctacacat gcacccagtg cggcaagagc |
| 1861 | ttccagtact cgcacaacct gagccgccat gccgtggtgc acacccgcga gaagccgcac |
| 1921 | gcctgcaagt ggtgcgagcg caggttcacg cagtccgggg acctgtacag acacattcgc |
| 1981 | aagttccact gtgagttggt gaactccttg tcggtcaaaa gcgaagcact gagcttgcct |
| 2041 | actgtcagag actggacctt agaagatagc tctcaagaac tttggaaata attttatata |
| 2101 | tatataaata atatatatat atatacatat atataaatag atctctatat agttgtggta |
| 2161 | cggtctaaaa gcagtcttgt ttcctggaaa taaaaagttg ggatattaac ttgtttttgc |
| 2221 | actttagaat agcatgagaa tctcactaat ttagcattct gataaaagaa actttagagc |
| 2281 | aagtcagaat agagaggtgt ttttcctttg aggggatagg ggaagtaagc caataagaac |
| 2341 | cttttaaaca aatcgtcctg tcacaaaatg ctttcatatg gcttaatttt gtcaacactg |
| 2401 | cattgtcttt tgagctcttt tttccccccc aacaaagttt ttttgttttt tgtttttttt |
| 2461 | tttaagtaga aattccctcc agttttatta gcctctttat atgtctcaaa ttgcatgaat |
| 2521 | tttttctggc tgttggaaac ctgaatgctt ttagacccaa atggaaaatt tctgaaatgc |
| 2581 | tggattatct atttttaaac aagcagttga cttaaaactt tctgtggcaa cttctggttt |
| 2641 | tctgacagtt cccagtgaga gaaatgctga aagtacactg ggatcactgg gacactgtct |
| 2701 | tatgaaggtt tgcttgggat gaaaaaggat attgcagctt cagcagtgtt gaactgtgtg |
| 2761 | tttaaaaatg tgaattactg ttattgtata ctgtaattga ttacatgggc tgggggggtg |
| 2821 | tcaaagaact tgacaggttg tgttgatgct cttagttgag tcttgaaaag taaatattaa |
| 2881 | cgctacagaa atgcatgagt ttcaatatat tttttgtctt tgtttgcatt gtataacttt |
| 2941 | aacgagtgag tttaaaatta tttaatttcc ttagaaaaat agcaccattt ggaaaaaaaa |
| 3001 | actggtgtta tgaagaacgt aaatgcactg tttttatttt tattttatat aatttaaatt |
| 3061 | gactttccca ctgtctttaa gttgaaactg ttaagctgaa taaaaactta agctgcaaat |
| 3121 | tgataacttc gctacataac aaggaaaata taaatgttta caaacagctt aaagatttgc |
| 3181 | atgtgcagtg tgcatttata acaaacttct aattgcacaa aacccatgcc agctcagagt |
| 3241 | ttaggtgtac acatttaccc agttgagcgt tcttagaata actactgcac aagttgacaa |
| 3301 | taggtcgttc tctctttttt tttgtttgct ccctttttct ttttctcccc ttcctcctta |
| 3361 | ccctccctcc cttactctcc ccccccacca ccaccctcca cccccaactc atgaaaagat |
| 3421 | tctatggact gaaaaagccc caggctgaaa ggactggact gccttgattg acatggggaa |
| 3481 | gggggttagt agactatgtg gattgcggca gcagaggctg cagcctaacg tgtggtttta |
| 3541 | atgaccagca cgcaaggcaa aagcattttg cacagtgttt gttttcctgt cttgcactta |
| 3601 | caaataaggt ctatgggagt agcatggaaa acgtttgctg tttttccctt ttttttttaa |
| 3661 | ttgcttttgt ttaaaatttg atcgccttaa ctactgtaaa catagcctat ttttgtgctt |
| 3721 | aagatactga atggaaaact ccattgtgtg ttgctggact gttttggaaa tatttggtta |
| 3781 | aatgtgtgtt aatttggctg taatggcatt taaagcaaac aaacaaacaa aaaaagctgt |
| 3841 | gaaaatggcc ttggagcatt atctttagtt acttgaagag tttctagttt ttttaaaata |
| 3901 | cagtttatgt taaaataatt tttattaatt tagagaagac aatcaatgtc tgtgagaaaa |
| 3961 | cggactttct tttggatttt ctttttgtgg tcattgtgag tgattgcttt ttccttttct |
| 4021 | tagtttcaca ttcttccttt gttctaaaac ttagactgac atctagcttt gacaatcata |
| 4081 | gtatgtttta ttttcctgag ggggaataac ttataatgct gtttagtttt gtactattgg |
| 4141 | tgtgttggtg aatttttaaa ctgtgtgcta actgcaataa attatatgaa ctgagaaaaa |
| 4201 | aaaaaaaaaa aaaaaaaaaa aaaaaaaaaa aaaaaaaaaa aaaa |

Id Proteins.

Id (inhibitor of DNA binding or inhibitor of differentiation) proteins belong to the helix-loop-helix (HLH) protein superfamily that is composed of seven currently known subclasses. They function through binding and sequestration of basic HLH (bHLH) transcription factors, thus preventing DNA binding and transcriptional activation of target genes (Norton et al., 1998, Trends Cell Biol 8, 58-65; herein incorporated by reference in its entirety). The dimerization of basic HLH proteins is necessary for their binding to DNA at the canonical E-box (CANNTG; SEQ ID NO: 245) or N-box (CACNAG; SEQ ID NO: 246) recognition sequences. Id proteins lack the basic domain necessary for DNA binding, and act primarily as dominant-negative regulators of bHLH transcription factors by sequestering and/or preventing DNA binding of ubiquitously expressed (e.g., E12, E47, E2-2) or cell-type-restricted (e.g., Tal-1, MyoD) factors. Four members of the Id protein family (Id1 to Id4) have been identified in mammals. Id proteins share a highly homologous HLH region, but have divergent sequences elsewhere.

Id2 enhances cell proliferation by promoting the transition from G1 to S phase of the cell cycle. Id proteins are abundantly expressed in stem cells, for example, neural stem cells before the decision to commit towards distinct neural lineages (Iavarone and Lasorella, 2004, Cancer Lett 204, 189-196; Perk et al., 2005, Nat Rev Cancer 5, 603-614; each herein incorporated by reference in its entirety). In stem cells, Id proteins act to maintain the undifferentiated and proliferative phenotype (Ying et al., 2003, Cell 115, 281-292; herein incorporated by reference in its entirety). Id expression is strongly reduced in mature cells from the central nervous system (CNS) but they accumulate at very high levels in neural cancer (Iavarone and Lasorella, 2004, Cancer Lett 204, 189-196; Lasorella et al., 2001, Oncogene 20, 8326-8333; each herein incorporated by reference in its entirety).

Id proteins act as negative regulators of differentiation, and depending on the specific cell lineage and developmental stage of the cell, Id proteins can act as positive regulators. Because bHLH proteins are mainly involved in the regulation of the expression of tissue specific and cell cycle related genes, Id-mediated sequestration or repression of bHLH proteins serves to block differentiation and to promote cell cycle activation. Accordingly, Id proteins have been shown to have biological roles as coordinators of different cellular processes, such as cell-fate determination, proliferation, cell-cycle regulation, angiogenesis, and cell migration. In some embodiments, the invention provides new methods for inhibiting proliferation of a neoplastic cell and for inhibiting angiogenesis in tumor tissue

The Biology of Human Malignant Brain Tumors.

High-grade gliomas, which include anaplastic astrocytoma (AA) and Glioblastoma Multiforme (GBM), are the most common intrinsic brain tumors in adults and are almost invariably lethal, largely as a result of their lack of responsiveness to current therapy (Legler et al., 200. J Natl Cancer Inst 92:77 A-8; herein incorporated by reference in its entirety). High-grade gliomas are the most common brain tumors in humans and are essentially incurable (A4; herein incorporated by reference in its entirety). The biological features that confer aggressiveness to human glioma are tissue invasion, neo-vascularization, marked increase in proliferation and resistance to cell death. Just as the ability to metastasize identifies the highest degree of malignancy in epithelial tumors, the defining hallmarks of aggressiveness of glioblastoma multiforme (GBM) are local. invasion and neoangiogenesis (A5, A6; each herein incorporated by reference in its entirety). Drivers of these phenotypic traits include intrinsic autocrine signals produced by brain tumor cells to invade the adjacent normal brain and stimulate formation of new blood vessels (A7; herein incorporated by reference in its entirety). It has been suggested that GBM re-engages pre-established ontogenetic motility and invasion signals that normally operate in neural stem cells and immature progenitors (A8; herein incorporated by reference in its entirety). A recently established notion postulates that neoplastic transformation in the central nervous system (CNS) converts neural stem cells into cell types manifesting a mesenchymal phenotype, a state associated with uncontrolled ability to invade and stimulate angiogenesis (A1, A2; each herein incorporated by reference in its entirety).

Differentiation along the mesenchymal lineage is virtually undetectable in the normal neural tissue during development. Global gene expression studies have established that over-expression of a “mesenchymal” gene expression signature (MGES) and loss of a proneural signature (PGES) co-segregate with the poorest prognosis group of glioma patients (A1; herein incorporated by reference in its entirety) (for simplicity, we will refer to the MGES+/PGES− signature as the mesenchymal phenotype of high-grade gliomas). It is unclear whether drift towards the mesenchymal lineage is exclusively an aberrant event that occurs during brain tumor progression or whether glioma cells recapitulate the rare mesenchymal plasticity of neural stem cells (A1-3, A9; each herein incorporated by reference in its entirety). More importantly, the molecular events that trigger activation/suppression of the MGES and PGES signatures and impart an intrinsically aggressive phenotype to glioma cells remain unknown.

Accordingly, Gene Expression Profile (GEP) studies of malignant glioma indicate that the expression of mesenchymal and angiogenesis-associated genes is associated with the worst prognosis (Freije, et al., 2004. Cancer Res 64:6503-10; Góddard et al., 2003. Cancer Res 63:6613-25; Liang et al., 2005. Proc Natl Acad Sci USA 102:5814-9; Nigro et al., 2005. Cancer Res 65:1678-86; each herein incorporated by reference in its entirety). Recently, glioma samples have been segregated into three groups with distinctive GEP signatures, displaying expression of genes characteristic of neural tissues (proneural), proliferating cells (proliferative) or mesenchymal tissues (mesenchymal) (Phillips et al., 2006. Cancer Cell 9:157-73; herein incorporated by reference in its entirety). Malignant gliomas in the mesenchymal group express genes linked with the most aggressive properties of GBM tumors (migration, invasion and angiogenesis) and invariably coincide with disease recurrence. The EXAMPLES discussed herein confirmed that molecular classification of gliomas effectively predicts clinical outcome. However, a major open challenge is the mapping and modeling of the regulatory programs responsible for the differential regulation of the three distinct expression signatures, each marking a specific cellular phenotype. In this proposal, we use combinations of computational and experimental approaches to unravel and validate the transcriptional and post translational interaction networks that drive the Mesenchymal Gene Expression Signature of high-grade glioma (MGES).

Maintenance of brain cells in a state referred to as “mesenchymal” is believed to be the cause of high-grade gliomas, the most common form of brain tumor in humans. For example, a pair of genes, Stat3 and C/EBPβ, can initiate and maintain the characteristics of the most common high-grade gliomas. Stat3 and C/EBP/3 are both transcription factors, meaning that they regulate the function of other genes. In so doing, Stat3, and C/EBPβ are master regulators of the mesenchymal state of brain cells which is the signature of human glioma. Therefore they are potential drug targets for the treatment of high-grade glioma. In some embodiments, co-expression of Stat3 and C/EBPβ in neural stem cells (brain cells that are naïve, otherwise called undifferentiated) is sufficient to initiate expression of the mesenchymal set of genes, suppress proneural genes, and trigger invasion and a malignant mesenchymal phenotype in the mouse indicating that these two genes can be causal for glioma. In some embodiments, silencing of these two transcription factors depletes glioma stem cells and cell lines of mesenchymal attributes and greatly impairs their ability to invade, perhaps indicating that silencing these genes help treat glioma. As discussed in the examples herein, independent immunohistochemistry experiments in 62 human glioma specimens show that concurrent expression of Stat3 and C/EBP is significantly associated with the expression of mesenchymal proteins and is an accurate predictor of poorest outcome in glioma patients.

In some embodiments, Stat3 and C/EBP are potential drug targets for the treatment of high-grade gliomas, with either small-molecule pharmaceuticals or gene-therapy strategies such as interfering RNAs. For example, diagnostic procedures can be designed to take advantage of the knowledge that Stat3 and C/EBP are regulators of human high-grade-gliomas. In some embodiments, measuring Stat3 and C/EBP expression can be a predictor of poorest outcome in glioma patients. This can be used early as a diagnostic indicator for the development of glioma.

Cell Regulatory Network Reverse Engineering.

Genome-scale approaches were recently applied to dissect regulatory networks in Eukaryotic organisms (Zhu et al., 2007. Genes Dev 21:1010-24; herein incorporated by reference in its entirety). These studies have shown that large-scale screens can be used to infer molecular interaction networks, with gene products represented as nodes and interactions as edges in a graph. Analysis of yeast networks (Barabasi and Oltavi, 2004. Nat Rev Genet. 5:101-13; herein incorporated by reference in its entirety), further validated in a mammalian context (Basso et al., 2005. Nat Genet. 37:382-90; herein incorporated by reference in its entirety), revealed that a relatively small number of key genes (hubs) regulate a large number of interactions, generating intense debate on the scale-free nature of these networks. Additionally, it has been shown that somatic lesions involved in tumorigenesis affect central hubs (Goh et al., 2007. Proc Natl Acad Sci USA 104:8685-90; herein incorporated by reference in its entirety). Master Regulators (MRs) are the regulatory hubs (transcriptional and post-translational) whose alteration is necessary and/or sufficient to implement a specific phenotypic transition (Lim et al., 2009. Pac Symp Biocomput 14:504-515; herein incorporated by reference in its entirety). Without being bound by theory, the combinatorial interaction of multiple, non-specific MRs yield high specificity in the control of individual programs associated with tumorigenesis and tumor aggressiveness. We thus plan to study the role of MRs and their combinatorial interplay in effecting the MGES that confers aggressiveness and recurrence to high-grade glioma.

The ARACNe and MINDy algorithms to reconstruct regulatory networks driving the mesenchymal signature of high-grade glioma.

ARACNe (Algorithm for the Reconstruction of Accurate Cellular Networks) is an established approach for the reverse engineering of transcriptional interactions from large GEP datasets (Basso et al., 2005. Nat Genet. 37:382-90; Margolin et al., 2006. BMC Bioinformatics 7 Suppl 1:S7; each herein incorporated by reference in its entirety). The main feature of this analytical tool is the use of the Mutual Information (MI) to identify candidate TF-target interactions. Indirect interactions are eliminated using the Data Processing Inequality (DPI), a well-known theoretical property of MI. As shown in several published studies and further demonstrated in the preliminary results section, ARACNe-inferred TF-target interactions have a high probability of corresponding to bona fide physical interactions. ARACNe was first used to dissect transcriptional interactions in human B cells, with experimental validation of C-MYC targets (Basso et al., 2005. Nat Genet. 37:382-90; herein incorporated by reference in its entirety). Additional studies in T cells, peripheral leukocytes, and rat brain tissue have confirmed a 70% to 90% validation rate of the ARACNe inferred targets for a wide range of TFs by Chromatin ImmunoPrecipitation assays (ChIP) (Palomero et al., 2006. Proc Natl Acad Sci USA 103:18261-6; herein incorporated by reference in its entirety). Software implementing ARACNe was downloaded by over 4,000 distinct researchers and has been referenced in ˜150 publications (Google Scholar), many of them providing independent validation of the method. Two ARACNe publications were selected by the Faculty of 1,000 (Basso et al., 2005. Nat Genet. 37:382-90; Margolin et al., 2006. Nat Protoc 1:662-71; each herein incorporated by reference in its entirety). Preliminary work using GBM microarray expression profile data (see EXAMPLES discussed herein) where ARACNe was developed indicates that the method is effective in heterogeneous cell populations. While cellular heterogeneity can increase the number of interactions missed by the approach (false-negatives), it does not introduce incorrect interactions (false positives). This is addressed in the Preliminary Data section where ARACNe-inferred TFs-targets interactions in neural tissue are validated.

Modulator Inference by Network Dynamics (MINDy) is the first algorithm able to accurately infer genome-wide repertoires of post-translational regulators of TF activity (Mani et al., 2008. Molecular Systems Biology 4:169-179; Wang et al., 2009. Pacific Symposium on Biocomputing 14:264-275; Wang et al., 2006. Lecture Notes in Computer Science 3909:348-362; Wang, K., M. Saito, B. Bisikirska, M. Alvarez, W. K. Lim, P. Rajbhandari, Q. Shen, I. Nemenman, K. Basso, A. A. Margolin, U. Klein, R. Dalla Favera, and A. Califano. 2009. Genome-wide identification of transcriptional network modulators in human B cells, Nature Biotechnology 27: 829-837; each herein incorporated by reference in its entirety). MINDy results have been used to infer (a) causal lesions, (b) drug mechanism of action in hematopoietic malignancies (Mani et al., 2008. Molecular Systems Biology 4:169-179; herein incorporated by reference in its entirety), and (c) to dissect the interface between signaling and transcriptional processes in B cells (Wang et al., 2009. Pacific Symposium on Biocomputing 14:264-275; herein incorporated by reference in its entirety). Inferences were biochemically validated. See EXAMPLES 2-5 for further detail.

DNA and Amino Acid Manipulation Methods

The invention utilizes conventional molecular biology, microbiology, and recombinant DNA techniques available to one of ordinary skill in the art. Such techniques are well known to the skilled worker and are explained fully in the literature. See, e.g., Maniatis, Fritsch & Sambrook, “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986): B. Perbal, “A Practical Guide to Molecular Cloning” (1984), and Sambrook, et al., “Molecular Cloning: a Laboratory Manual” (2001); herein incorporated by reference in its entirety.

One skilled in the art can obtain a Mesenchymal-Gene-Expression-Signature (MGES) protein or a variant thereof (e.g., Stat3, C/EBPβ, C/EBPδ, RunX1, FosL2, bHLH-B2, ZNF238), in several ways, which include, but are not limited to, isolating the protein via biochemical means or expressing a nucleotide sequence encoding the protein of interest by genetic engineering methods.

In another aspect, the invention provides for MGES molecule or variants thereof that are encoded by nucleotide sequences. As used herein, a “MGES molecule” refers to a Stat3, C/EBPβ, C/EBPδ, RunX1, FosL2, bHLH-B2, or ZNF238 protein. The MGES molecule can be a polypeptide encoded by a nucleic acid (including genomic DNA, complementary DNA (cDNA), synthetic DNA, as well as any form of corresponding RNA). For example, a MGES molecule can be encoded by a recombinant nucleic acid encoding human MGES protein. The MGES molecules of the invention can be obtained from various sources and can be produced according to various techniques known in the art. For example, a nucleic acid that encodes a MGES molecule can be obtained by screening DNA libraries, or by amplification from a natural source. The MGES molecules of the invention can be produced via recombinant DNA technology and such recombinant nucleic acids can be prepared by conventional techniques, including chemical synthesis, genetic engineering, enzymatic techniques, or a combination thereof. A MGES molecule of this invention can also encompasses variants of the human MGES proteins (e.g., Stat3, C/EBPβ, C/EBPδ, RunX1, FosL2, bHLH-B2, or ZNF238). The variants can comprise naturally-occurring variants due to allelic variations between individuals (e.g., polymorphisms), mutated alleles related to hair growth or texture, or alternative splicing forms

In some embodiments, the nucleic acid is expressed in an expression cassette, for example, to achieve overexpression in a cell. The nucleic acids of the invention can be an RNA, cDNA, cDNA-like, or a DNA of interest in an expressible format, such as an expression cassette, which can be expressed from the natural promoter or an entirely heterologous promoter. The nucleic acid of interest can encode a protein, and may or may not include introns.

Protein variants can involve amino acid sequence modifications. For example, amino acid sequence modifications fall into one or more of three classes: substitutional, insertional or deletional variants. Insertions can include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site-specific mutagenesis of nucleotides in the DNA encoding the protein, thereby producing DNA encoding the variant, and thereafter expressing the DNA in recombinant cell culture.

Techniques for making substitution mutations at predetermined sites in DNA having a known sequence are well known, for example M13 primer mutagenesis and PCR mutagenesis. Amino acid substitutions can be single residues, but can occur at a number of different locations at once. In some non-limiting embodiments, insertions can be on the order of about from 1 to about 10 amino acid residues, while deletions can range from about 1 to about 30 residues. Deletions or insertions can be made in adjacent pairs (for example, a deletion of about 2 residues or insertion of about 2 residues). Substitutions, deletions, insertions, or any combination thereof can be combined to arrive at a final construct. The mutations cannot place the sequence out of reading frame and cannot create complementary regions that can produce secondary mRNA structure. Substitutional variants are those in which at least one residue has been removed and a different residue inserted in its place.

Expression Systems

Bacterial and Yeast Expression Systems.

In bacterial systems, a number of expression vectors can be selected. For example, when a large quantity of an MGES protein is needed for the induction of antibodies, vectors which direct high level expression of fusion proteins that are readily purified can be used. Non-limiting examples of such vectors include multifunctional E. coli cloning and expression vectors such as BLUESCRIPT (Stratagene). pIN vectors or pGEX vectors (Promega, Madison, Wis.) also can be used to express foreign polypeptide molecules as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

Plant and Insect Expression Systems.

If plant expression vectors are used, the expression of sequences encoding a MGES molecule can be driven by any of a number of promoters. For example, viral promoters such as the 35S and 19S promoters of CaMV can be used alone or in combination with the omega leader sequence from TMV. Alternatively, plant promoters such as the small subunit of RUBISCO or heat shock promoters, can be used. These constructs can be introduced into plant cells by direct DNA transformation or by pathogen-mediated transfection.

An insect system also can be used to express MGES molecules. For example, in one such system Autographa californica nuclear polyhedrosis virus (AcNPV) is used as a vector to express foreign genes in Spodoptera frugiperda cells or in Trichoplusia larvae. Sequences encoding a MGES molecule can be cloned into a non-essential region of the virus, such as the polyhedrin gene, and placed under control of the polyhedrin promoter. Successful insertion of MGES nucleic acid sequences will render the polyhedrin gene inactive and produce recombinant virus lacking coat protein. The recombinant viruses can then be used to infect S. frugiperda cells or Trichoplusia larvae in which MGES or a variant thereof can be expressed.

Mammalian Expression Systems.

An expression vector can include a nucleotide sequence that encodes a MGES molecule linked to at least one regulatory sequence in a manner allowing expression of the nucleotide sequence in a host cell. A number of viral-based expression systems can be used to express a MGES molecule or a variant thereof in mammalian host cells. The vector can be a recombinant DNA or RNA vector, and includes DNA plasmids or viral vectors. For example, if an adenovirus is used as an expression vector, sequences encoding a MGES molecule can be ligated into an adenovirus transcription/translation complex comprising the late promoter and tripartite leader sequence. Insertion into a non-essential E1 or E3 region of the viral genome can be used to obtain a viable virus which is capable of expressing a MGES molecule in infected host cells. Transcription enhancers, such as the Rous sarcoma virus (RSV) enhancer, can also be used to increase expression in mammalian host cells. In addition, a multitargeting interfering RNA molecule expressing viral vectors can be constructed based on, but not limited to, adeno-associated virus, retrovirus, adenovirus, lentivirus or alphavirus.

Regulatory sequences are well known in the art, and can be selected to direct the expression of a protein or polypeptide of interest (such as a MGES molecule) in an appropriate host cell as described in Goeddel, Gene Expression Technology: Methods in Enzymology 185, Academic Press, San Diego, Calif. (1990); herein incorporated by reference in its entirety. Non-limiting examples of regulatory sequences include: polyadenylation signals, promoters (such as CMV, ASV, SV40, or other viral promoters such as those derived from bovine papilloma, polyoma, and Adenovirus 2 viruses (Fiers, et al., 1973, Nature 273:113; Hager G L, et al., Curr Opin Genet Dev, 2002, 12(2):137-41; each herein incorporated by reference in its entirety) enhancers, and other expression control elements.

Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication.

For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (for example, a P2RY5 molecule) is stably integrated into the genome of eukaryotic cells (for example mammalian cells, such as cells from the end bulb of the hair follicle), resulting in the stable expression of transfected genes. An exogenous nucleic acid sequence can be introduced into a cell (such as a mammalian cell, either a primary or secondary cell) by homologous recombination as disclosed in U.S. Pat. No. 5,641,670, the entire contents of which are herein incorporated by reference.

A gene that encodes a selectable marker (for example, resistance to antibiotics or drugs, such as ampicillin, neomycin, G418, and hygromycin) can be introduced into host cells along with the gene of interest in order to identify and select clones that stably express a gene encoding a protein of interest. The gene encoding a selectable marker can be introduced into a host cell on the same plasmid as the gene of interest or can be introduced on a separate plasmid. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein molecule (for example, a MGES protein).

Cell Transfection and Culturing

Cell Transfection.

A eukaryotic expression vector can be used to transfect cells in order to produce proteins (for example, a MGES molecule) encoded by nucleotide sequences of the vector. Mammalian cells can contain an expression vector (for example, one that contains a gene encoding a MGES molecule) via introducing the expression vector into an appropriate host cell via methods known in the art.

A host cell strain can be chosen for its ability to modulate the expression of the inserted sequences or to process the expressed MGES polypeptide (such as, e.g., Stat3, C/EBPβ, C/EBPδ, RunX1, FosL2, bHLH-B2, ZNF238) in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; University Boulevard, Manassas, Va. 20110-2209) and can be chosen to ensure the correct modification and processing of the foreign protein.

An exogenous nucleic acid can be introduced into a cell via a variety of techniques known in the art, such as lipofection, microinjection, calcium phosphate or calcium chloride precipitation, DEAE-dextrin-mediated transfection, or electroporation. Electroporation is carried out at approximate voltage and capacitance to result in entry of the DNA construct(s) into cells of interest (such as cells of the end bulb of a hair follicle, for example dermal papilla cells or dermal sheath cells). Other methods used to transfect cells can also include modified calcium phosphate precipitation, polybrene precipitation, liposome fusion, and receptor-mediated gene delivery.

Cells to be genetically engineered can be primary and secondary cells obtained from various tissues, and include cell types which can be maintained and propagated in culture. Non-limiting examples of primary and secondary cells include epithelial cells, neural cells, endothelial cells, glial cells, fibroblasts, muscle cells (such as myoblasts) keratinocytes, formed elements of the blood (e.g., lymphocytes, bone marrow cells), and precursors of these somatic cell types. Vertebrate tissue can be obtained by methods known to one skilled in the art, such a punch biopsy or other surgical methods of obtaining a tissue source of the primary cell type of interest. A mixture of primary cells can be obtained from the tissue, using methods readily practiced in the art, such as explanting or enzymatic digestion (for examples using enzymes such as pronase, trypsin, collagenase, elastase dispase, and chymotrypsin). Biopsy methods have also been described in United States Patent Application Publication 2004/0057937 and PCT application publication WO 2001/32840, each of which are hereby incorporated by reference in its entirety.

Primary cells can be acquired from the individual to whom the genetically engineered primary or secondary cells are administered. However, primary cells can also be obtained from a donor, other than the recipient, of the same species. The cells can also be obtained from another species (for example, rabbit, cat, mouse, rat, sheep, goat, dog, horse, cow, bird, or pig). Primary cells can also include cells from an isolated vertebrate tissue source grown attached to a tissue culture substrate (for example, flask or dish) or grown in a suspension; cells present in an explant derived from tissue; both of the aforementioned cell types plated for the first time; and cell culture suspensions derived from these plated cells. Secondary cells can be plated primary cells that are removed from the culture substrate and replated, or passaged, in addition to cells from the subsequent passages. Secondary cells can be passaged one or more times. These primary or secondary cells can contain expression vectors having a gene that encodes a protein of interest (for example, a MGES molecule).

Cell Culturing.

Various culturing parameters can be used with respect to the host cell being cultured. Appropriate culture conditions for mammalian cells are well known in the art (Cleveland W L, et al., J Immunol Methods, 1983, 56(2): 221-234; herein incorporated by reference in its entirety) or can be determined by the skilled artisan (see, for example, Animal Cell Culture: A Practical Approach 2nd Ed., Rickwood, D. and Hames, B. D., eds. (Oxford University Press: New York, 1992); herein incorporated by reference in its entirety). Cell culturing conditions can vary according to the type of host cell selected. Commercially available medium can be utilized. Non-limiting examples of medium include, for example, Minimal Essential Medium (MEM, Sigma, St. Louis, Mo.); Dulbecco's Modified Eagles Medium (DMEM, Sigma); Ham's FIO Medium (Sigma); HyClone cell culture medium (HyClone, Logan, Utah); RPMI-1640 Medium (Sigma); and chemically-defined (CD) media, which are formulated for various cell types, e.g., CD-CHO Medium (Invitrogen, Carlsbad, Calif.).

The cell culture media can be supplemented as necessary with supplementary components or ingredients, including optional components, in appropriate concentrations or amounts, as necessary or desired. Cell culture medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that can be required at very low concentrations, usually in the micromolar range.

The medium also can be supplemented electively with one or more components from any of the following categories: (1) salts, for example, magnesium, calcium, and phosphate; (2) hormones and other growth factors such as, serum, insulin, transferrin, and epidermal growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example pluronic polyol; and (8) galactose. In some embodiments, soluble factors can be added to the culturing medium.

Cells suitable for culturing can contain introduced expression vectors, such as plasmids or viruses. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 201, Molecular Cloning, A Laboratory Manual, Cold Spring Harbor Press, Cold Spring Harbor, N.Y. and in F. M. Ausubel et al., 1989, Current Protocols in Molecular Biology, John Wiley & Sons, New York, N.Y.; each herein incorporated by reference in its entirety.

DNA and Polypeptides, Methods, and Purification Thereof

The present invention utilizes conventional molecular biology, microbiology, and recombinant DNA techniques available to one of ordinary skill in the art. Such techniques are well known to the skilled worker and are explained fully in the literature. See, e.g. “DNA Cloning: A Practical Approach,” Volumes 1 and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986): B. Perbal, “A Practical Guide to Molecular Cloning” (1984), and Sambrook, et al., “Molecular Cloning: a Laboratory Manual” (3^{rd} edition, 2001); each herein incorporated by reference in its entirety. One skilled in the art can obtain a protein encoded by an MGES gene (such as, e.g., Stat3, C/EBPβ, C/EBPδ, RunX1, FosL2, bHLH-B2, ZNF238) in several ways, which include, but are not limited to, isolating the protein via biochemical means or expressing a nucleotide sequence encoding the protein of interest by genetic engineering methods. For example, Stat3, C/EBPβ, C/EBPδ, RunX1, FosL2, bHLH-B2, ZNF238, or a variant thereof, can be obtained by purifying it from human cells expressing the same, or by direct chemical synthesis.

Host cells which contain a nucleic acid encoding an MGES polypeptide (such as, e.g., Stat3, C/EBPβ, C/EBPδ, RunX1, FosL2, bHLH-B2, ZNF238), and which subsequently express a protein encoded by an MGES gene (such as, e.g., Stat3, C/EBPβ, C/EBPδ, RunX1, FosL2, bHLH-B2, ZNF238), can be identified by various procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a nucleic acid encoding a MGES polypeptide (such as, e.g., Stat3, C/EBPβ, C/EBPδ, RunX1, FosL2, bHLH-B2, ZNF238) can be detected by DNA-DNA or DNA-RNA hybridization or amplification using probes or fragments of nucleic acids encoding a MGES polypeptide.

Amplification methods include, e.g., polymerase chain reaction, PCR (PCR PROTOCOLS, A GUIDE TO METHODS AND APPLICATIONS, ed. Innis, Academic Press, N.Y., 1990 and PCR STRATEGIES, 1995, ed. Innis, Academic Press, Inc., N.Y.; each herein incorporated by reference in its entirety), ligase chain reaction (LCR) (see, e.g., Wu, Genomics 4:560, 1989; Landegren, Science 241:1077, 1988; Barringer, Gene 89:117, 1990; each herein incorporated by reference in its entirety); transcription amplification (see, e.g., Kwoh, Proc. Natl. Acad. Sci. USA 86:1173, 1989; herein incorporated by reference in its entirety); and, self-sustained sequence replication (see, e.g., Guatelli, Proc. Natl. Acad. Sci. USA 87:1874, 1990; herein incorporated by reference in its entirety); Q Beta replicase amplification (see, e.g., Smith, J. Clin. Microbiol. 35:1477-1491, 1997; herein incorporated by reference in its entirety), automated Q-beta replicase amplification assay (see, e.g., Burg, Mol. Cell. Probes 10:257-271, 1996; herein incorporated by reference in its entirety) and other RNA polymerase mediated techniques (e.g., NASBA, Cangene, Mississauga, Ontario); see also Berger, Methods Enzymol. 152:307-316, 1987; U.S. Pat. Nos. 4,683,195 and 4,683,202; Sooknanan, Biotechnology 13:563-564, 1995; each herein incorporated by reference in its entirety.

A guide to the hybridization of nucleic acids is found in e.g., Sambrook, ed., Molecular Cloning: A Laboratory Manual (3^{rd} Ed.), Vols. 1-3, Cold Spring Harbor Laboratory, 2001; Current Protocols In Molecular Biology, Ausubel, ed. John Wiley & Sons, Inc., New York, 1997; Laboratory Techniques In Biochemistry And Molecular Biology: Hybridization With Nucleic Acid Probes, Part I. Theory and Nucleic Acid Preparation, Tijssen, ed. Elsevier, N.Y., 1993; each herein incorporated by reference in its entirety.

In some embodiments, a fragment of a nucleic acid of an MGES gene (such as, e.g., Stat3, C/EBPβ, C/EBPδ, RunX1, FosL2, bHLH-B2, ZNF238) can encompass any portion of at least about 8 consecutive nucleotides of either SEQ ID NOS: 232, 234, 236, 238, 240, 242, or 244. In some embodiments, the fragment can comprise at least about 10 consecutive nucleotides, at least about 15 consecutive nucleotides, at least about 20 consecutive nucleotides, or at least about 30 consecutive nucleotides of either SEQ ID NOS: 232, 234, 236, 238, 240, 242, or 244. Fragments can include all possible nucleotide lengths between about 8 and about 100 nucleotides, for example, lengths between about 15 and about 100 nucleotides, or between about 20 and about 100 nucleotides. Nucleic acid amplification-based assays involve the use of oligonucleotides selected from sequences encoding a polypeptide encoded by an MGES gene (such as, e.g., Stat3, C/EBPβ, C/EBPδ, RunX1, FosL2, bHLH-B2, ZNF238), to detect transformants which contain a nucleic acid encoding an MGES protein or polypeptide, e.g., Stat3, C/EBPβ, C/EBPδ, RunX1, FosL2, bHLH-B2, ZNF238.

Various techniques known in the art can be used to detect or quantify altered gene expression, RNA expression, or sequence, which include, but are not limited to, hybridization, sequencing, amplification, and/or binding to specific ligands (such as antibodies). Other suitable methods include allele-specific oligonucleotide (ASO), oligonucleotide ligation, allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, denaturing HLPC, melting curve analysis, heteroduplex analysis, RNase protection, chemical or enzymatic mismatch cleavage, ELISA, radio-immunoassays (RIA) and immuno-enzymatic assays (IEMA). Some of these approaches (such as SSCA and CGGE) are based on a change in electrophoretic mobility of the nucleic acids, as a result of the presence of an altered sequence. According to these techniques, the altered sequence is visualized by a shift in mobility on gels. The fragments can then be sequenced to confirm the alteration. Some other approaches are based on specific hybridization between nucleic acids from the subject and a probe specific for wild type or altered gene or RNA. The probe can be in suspension or immobilized on a substrate. The probe can be labeled to facilitate detection of hybrids. Some of these approaches are suited for assessing a polypeptide sequence or expression level, such as Northern blot, ELISA and RIA. These latter require the use of a ligand specific for the polypeptide, for example, the use of a specific antibody.

Embodiments of the invention provide for detecting whether expression of an MGES gene (such as, e.g., Stat3, C/EBPβ, C/EBPδ, RunX1, FosL2, bHLH-B2, or ZNF238) is altered. In some embodiments, the gene alteration can result in increased or reduced gene expression and/or activity. In some embodiments, the gene alteration can also result in increased or reduced protein expression and/or activity.

An alteration in a MGES gene locus (e.g., where Stat3, C/EBPβ, C/EBPδ, RunX1, FosL2, bHLH-B2, or ZNF238 are located) can be any form of mutation(s), deletion(s), rearrangement(s) and/or insertions in the coding and/or non-coding region of the locus, alone or in various combination(s). Mutations can include point mutations. Insertions can encompass the addition of one or several residues in a coding or non-coding portion of the gene locus. Insertions can comprise an addition of between 1 and 50 base pairs in the gene locus. Deletions can encompass any region of one, two or more residues in a coding or non-coding portion of the gene locus, such as from two residues up to the entire gene or locus. Deletions can affect smaller regions, such as domains (introns) or repeated sequences or fragments of less than about 50 consecutive base pairs; although larger deletions can occur as well. Rearrangement includes inversion of sequences.

The MGES gene locus alteration (such as, e.g., Stat3, C/EBPβ, C/EBPδ, RunX1, FosL2, bHLH-B2, or ZNF238) can result in amino acid substitutions, RNA splicing or processing, product instability, the creation of stop codons, frame-shift mutations, and/or truncated polypeptide production. The alteration can result in the production of a MGES polypeptide with altered function, stability, targeting or structure. The alteration can also cause a reduction in protein expression. In some embodiments, the alteration in a MGES gene locus can comprise a point mutation, a deletion, or an insertion in the MGES gene or corresponding expression product. The alteration can be determined at the level of the DNA, RNA, or polypeptide.

In some embodiments, the detecting comprises detecting in a biological sample whether there is a reduction in an mRNA encoding an MGES polypeptide, or a reduction in a MGES protein, or a combination thereof. In some embodiments, the detecting comprises detecting in a biological sample whether there is a reduction in an mRNA encoding an MGES polypeptide, or a reduction in a MGES protein, or a combination thereof. The presence of such an alteration is indicative of the presence or predisposition to a nervous system cancer (e.g., a glioma). The presence of an alteration in an MGES gene encoding an MGES polypeptide (such as, e.g., Stat3, C/EBPβ, C/EBPδ, RunX1, FosL2, bHLH-B2, or ZNF238) in the sample is detected through the genotyping of a sample, for example via gene sequencing, selective hybridization, amplification, gene expression analysis, or a combination thereof.

Methods for detecting and quantifying MGES polypeptides (such as, e.g., Stat3, C/EBPβ, C/EBPδ, RunX1, FosL2, bHLH-B2, or ZNF238 polypeptides) and MGES polynucleotides (e.g., Stat3, C/EBPβ, C/EBPδ, RunX1, FosL2, bHLH-B2, or ZNF238 polynucleotides) in biological samples are known the art. For example, protocols for detecting and measuring the expression of a polypeptide encoded by an MGES gene, such as Stat3, C/EBPβ, C/EBPδ, RunX1, FosL2, bHLH-B2, or ZNF238, using either polyclonal or monoclonal antibodies specific for the polypeptide are well established. Non-limiting examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a polypeptide encoded by an MGES gene (e.g, Stat3, C/EBPβ, C/EBPδ, RunX1, FosL2, bHLH-B2, or ZNF238) can be used, or a competitive binding assay can be employed. In some embodiments, expression or over-expression of an MGES gene product (e.g., a MGES polypeptide or MGES mRNA) can be determined. In some embodiments, a biological sample comprises, a blood sample, serum, cells (including whole cells, cell fractions, cell extracts, and cultured cells or cell lines), tissues (including tissues obtained by biopsy), body fluids (e.g., urine, sputum, amniotic fluid, synovial fluid), or from media (from cultured cells or cell lines). The methods of detecting or quantifying MGES polynucleotides (such as, e.g., Stat3, C/EBPβ, C/EBPδ, RunX1, FosL2, bHLH-B2, or ZNF238) include, but are not limited to, amplification-based assays with signal amplification) hybridization based assays and combination amplification-hybridization assays. For detecting and quantifying MGES polypeptides (such as, e.g., Stat3, C/EBPβ, C/EBPδ, RunX1, FosL2, bHLH-B2, or ZNF238), an exemplary method is an immunoassay that utilizes an antibody or other binding agents that specifically bind to a MGES polypeptide (such as, e.g., Stat3, C/EBPβ, C/EBPδ, RunX1, FosL2, bHLH-B2, or ZNF238) or epitope of such, for example, ELISA or RIA assays.

Labeling and conjugation techniques are known by those skilled in the art and can be used in various nucleic acid and amino acid assays. Methods for producing labeled hybridization or PCR probes for detecting sequences related to nucleic acid sequences encoding an MGES protein (such as, e.g., Stat3, C/EBPβ, C/EBPβ, RunX1, FosL2, bHLH-B2, or ZNF238), include, but are not limited to, oligolabeling, nick translation, end-labeling, or PCR amplification using a labeled nucleotide. Alternatively, a nucleic acid sequence encoding a polypeptide encoded by an MGES gene can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, and/or magnetic particles.

Host cells transformed with a nucleic acid sequence encoding an MGES polypeptide (such as, e.g., Stat3, C/EBPβ, C/EBPδ, RunX1, FosL2, bHLH-B2, or ZNF238), can be cultured under conditions suitable for the expression and recovery of the protein from cell culture. The polypeptide produced by a transformed cell can be secreted or contained intracellularly depending on the sequence and/or the vector used. Expression vectors containing a nucleic acid sequence encoding an MGES polypeptide can be designed to contain signal sequences which direct secretion of soluble polypeptide molecules encoded by an MGES gene (such as, e.g., Stat3, C/EBPβ, C/EBPδ, RunX1, FosL2, bHLH-B2, or ZNF238), through a prokaryotic or eukaryotic cell membrane, or which direct the membrane insertion of a membrane-bound polypeptide molecule encoded by an MGES gene.

Other constructions can also be used to join a gene sequence encoding an MGES polypeptide (such as, e.g., Stat3, C/EBPβ, C/EBPδ, RunX1, FosL2, bHLH-B2, or ZNF238) to a nucleotide sequence encoding a polypeptide domain which would facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Including cleavable linker sequences (i.e., those specific for Factor Xa or enterokinase (Invitrogen, San Diego, Calif.)) between the purification domain and a polypeptide encoded by an MGES gene also can be used to facilitate purification. One such expression vector provides for expression of a fusion protein containing a polypeptide encoded by an MGES gene (such as, e.g., Stat3, C/EBPβ, C/EBPδ, RunX1, FosL2, bHLH-B2, or ZNF238) and 6 histidine residues preceding a thioredoxin or an enterokinase cleavage site. The histidine residues facilitate purification by immobilized metal ion affinity chromatography, while the enterokinase cleavage site provides a means for purifying the polypeptide encoded by an MGES gene.

An MGES polypeptide (such as, e.g., Stat3, C/EBPβ, C/EBPδ, RunX1, FosL2, bHLH-B2, or ZNF238) can be purified from any human or non-human cell which expresses the polypeptide, including those which have been transfected with expression constructs that express an MGES protein. A purified MGES polypeptide (such as, e.g., Stat3, C/EBPβ, C/EBPδ, RunX1, FosL2, bHLH-B2, or ZNF238) can be separated from other compounds which normally associate with the MGES polypeptide in the cell, such as certain proteins, carbohydrates, or lipids, using methods practiced in the art. Non-limiting methods include size exclusion chromatography, ammonium sulfate fractionation, affinity chromatography, ion exchange chromatography, and preparative gel electrophoresis.

Nucleic acid sequences comprising an MGES gene (such as, e.g., Stat3, C/EBPβ, C/EBPδ, RunX1, FosL2, bHLH-B2, or ZNF238) that encode a polypeptide can be synthesized, in whole or in part, using chemical methods known in the art. Alternatively, an MGES polypeptide can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431 A Peptide Synthesizer (Perkin Elmer). Optionally, fragments of MGES polypeptides can be separately synthesized and combined using chemical methods to produce a full-length molecule. In some embodiments, a fragment of a nucleic acid sequence that comprises an MGES gene (such as, e.g., Stat3, C/EBPβ, C/EBPβ, RunX1, FosL2, bHLH-B2, or ZNF238) can encompass any portion of at least about 8 consecutive nucleotides of SEQ ID NO: 232, 234, 236, 238, 240, 242, or 244. In some embodiments, the fragment can comprise at least about 10 nucleotides, at least about 15 nucleotides, at least about 20 nucleotides, or at least about 30 nucleotides of SEQ ID NO: 232, 234, 236, 238, 240, 242, or 244. Fragments include all possible nucleotide lengths between about 8 and about 100 nucleotides, for example, lengths between about 15 and about 100 nucleotides, or between about 20 and about 100 nucleotides.

An MGES fragment can be a fragment of an MGES protein, such as, e.g., Stat3, C/EBPβ, C/EBPδ, RunX1, FosL2, bHLH-B2, and ZNF238. For example, the MGES fragment can encompass any portion of at least about 8 consecutive amino acids of SEQ ID NO: 231, 233, 235, 237, 239, 241, or 243. The fragment can comprise at least about 10 consecutive amino acids, at least about 20 consecutive amino acids, at least about 30 consecutive amino acids, at least about 40 consecutive amino acids, a least about 50 consecutive amino acids, at least about 60 consecutive amino acids, at least about 70 consecutive amino acids, or at least about 75 consecutive amino acids of SEQ ID NO: 231, 233, 235, 237, 239, 241, or 243. Fragments include all possible amino acid lengths between about 8 and 100 about amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids.

A synthetic peptide can be substantially purified via high performance liquid chromatography (HPLC). The composition of a synthetic MGES polypeptide can be confirmed by amino acid analysis or sequencing. Additionally, any portion of an amino acid sequence comprising a protein encoded by an MGES gene (e.g., Stat3, C/EBPβ, C/EBPδ, RunX1, FosL2, bHLH-B2, and ZNF238) can be altered during direct synthesis and/or combined using chemical methods with sequences from other proteins to produce a variant polypeptide or a fusion protein.

The invention further encompasses methods for using a protein or polypeptide encoded by a nucleic acid sequence of an MGES gene, such as the sequences shown in SEQ ID NOS: 231, 233, 235, 237, 239, 241, or 244. In some embodiments, the polypeptide can be modified, such as by glycosylations and/or acetylations and/or chemical reaction or coupling, and can contain one or several non-natural or synthetic amino acids. An example of an MGES polypeptide has the amino acid sequence shown in either SEQ ID NO: 231, 233, 235, 237, 239, 241, or 244. In certain embodiments, the invention encompasses variants of a human protein encoded by an MGES gene (such as, e.g., Stat3, C/EBPβ, C/EBPδ, RunX1, FosL2, bHLH-B2, and ZNF238). Such variants can include those having at least from about 46% to about 50% identity to SEQ ID NO: 231, 233, 235, 237, 239, 241, or 244, or having at least from about 50.1% to about 55% identity to SEQ ID NO: 231, 233, 235, 237, 239, 241, or 244, or having at least from about 55.1% to about 60% identity to SEQ ID NO: 231, 233, 235, 237, 239, 241, or 244, or having from at least about 60.1% to about 65% identity to SEQ ID NO: 231, 233, 235, 237, 239, 241, or 244, or having from about 65.1% to about 70% identity to SEQ ID NO: 231, 233, 235, 237, 239, 241, or 244, or having at least from about 70.1% to about 75% identity to SEQ ID NO: 231, 233, 235, 237, 239, 241, or 244, or having at least from about 75.1% to about 80% identity to SEQ ID NO: 231, 233, 235, 237, 239, 241, or 244, or having at least from about 80.1% to about 85% identity to SEQ ID NO: 231, 233, 235, 237, 239, 241, or 244, or having at least from about 85.1% to about 90% identity to SEQ ID NO: 231, 233, 235, 237, 239, 241, or 244, or having at least from about 90.1% to about 95% identity to SEQ ID NO 231, 233, 235, 237, 239, 241, or 244, or having at least from about 95.1% to about 97% identity to SEQ ID NO: 231, 233, 235, 237, 239, 241, or 244, or having at least from about 97.1% to about 99% identity to SEQ ID NO: 231, 233, 235, 237, 239, 241, or 244.

Identifying MGES Modulating Compounds

The invention provides methods for identifying compounds which can be used for controlling and/or regulating mesenchymal signature genes (i.e., MGES genes such as Stat3, C/EBPβ, C/EBPδ, RunX1, FosL2, bHLH-B2, ZNF238) of nervous system cancers. In addition, the invention provides methods for identifying compounds which can be used for the treatment of a nervous system cancers, such as malignant glioma. The methods can comprise the identification of test compounds or agents (e.g., peptides (such as antibodies or fragments thereof), small molecules, nucleic acids (such as siRNA or antisense RNA), or other agents) that can bind to a MGES polypeptide molecule and/or have a stimulatory or inhibitory effect on the biological activity of MGES or its expression, and subsequently determining whether these compounds can regulate mesenchymal signature genes of nervous system cancers in a subject or can have an effect on tumor growth in an in vitro or an in vivo assay (i.e., examining whether there is a decrease in tumor growth).

As used herein, a “MGES modulating compound” refers to a compound that interacts with an MGES transcription factor and modulates its DNA binding activity and/or its expression. The compound can either increase a MGES' activity or expression (e.g., Stat3, C/EBPβ, C/EBPδ, RunX1, FosL2, bHLH-B2, ZNF238). Conversely, the compound can decrease a MGES' activity or expression (e.g., Stat3, C/EBPβ, C/EBPδ, RunX1, FosL2, bHLH-B2, ZNF238). The compound can be a MGES inhibitor, agonist, or a MGES antagonist. Some non-limiting examples of MGES modulating compounds include peptides (such as MGES peptide fragments, or antibodies or fragments thereof), small molecules, and nucleic acids (such as MGES siRNA or antisense RNA specific for a MGES nucleic acid). Agonists of a MGES molecule can be molecules which, when bound to a MGES (such as Stat3, C/EBPβ, C/EBPδ, RunX1, FosL2, bHLH-B2, ZNF238) increase the expression, or increase or prolong the activity of a MGES molecule. Agonists of a MGES include, but are not limited to, proteins, nucleic acids, small molecules, or any other molecule which activates MGES. Antagonists of a MGES molecule can be molecules which, when bound to MGES or a variant thereof, decrease the amount or the duration of the activity of a MGES molecule. Antagonists include proteins, nucleic acids, antibodies, small molecules, or any other molecule which decrease the activity of MGES.

The term “modulate”, as it appears herein, refers to a change in the activity or expression of a MGES molecule (such as, Stat3, C/EBPβ, C/EBPδ, RunX1, FosL2, bHLH-B2, ZNF238). For example, modulation can cause an increase or a decrease in protein activity, binding characteristics, or any other biological, functional, or immunological properties of a MGES molecule.

In some embodiments, a MGES modulating compound can be a peptide fragment of a MGES protein that binds to the MGES or the upstream DNA region where the MGES transcription factor binds to. Peptide fragments can be obtained commercially or synthesized via liquid phase or solid phase synthesis methods (Atherton et al., (1989) Solid Phase Peptide Synthesis: a Practical Approach. IRL Press, Oxford, England; herein incorporated by reference in its entirety). The MGES peptide fragments can be isolated from a natural source, genetically engineered, or chemically prepared. These methods are well known in the art.

A MGES modulating compound can also be a protein, such as an antibody (monoclonal, polyclonal, humanized, and the like), or a binding fragment thereof, directed against the MGES. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402; each herein incorporated by reference in its entirety). Antibodies can be obtained commercially, custom generated, or synthesized against an antigen of interest according to methods established in the art (e.g., see Beck et al., Nat Rev Immunol. 2010 May; 10(5):345-52; Chan et al., Nat Rev Immunol. 2010 May; 10(5):301-16; and Kontermann, Curr Opin Mol. Ther. 2010 April; 12(2):176-83, each of which are incorporated by reference in their entireties).

Inhibition of RNA encoding a MGES molecule can effectively modulate the expression of the MGES gene from which the RNA is transcribed. Inhibitors are selected from the group comprising: siRNA, interfering RNA or RNAi; dsRNA; RNA Polymerase III transcribed DNAs; shRNAs; ribozymes; and antisense nucleic acid, which can be RNA, DNA, or artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding a MGES polypeptide can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59; each herein incorporated by reference in its entirety).

siRNA comprises a double stranded structure containing from about 15 to about 50 base pairs, for example from about 21 to about 25 base pairs, and having a nucleotide sequence identical or nearly identical to an expressed target gene or RNA within the cell. Antisense nucleotide sequences include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The MGES modulating compound can contain ribonucleotides, deoxyribonucleotides, synthetic nucleotides, or any suitable combination such that the target RNA and/or gene is inhibited. In addition, these forms of nucleic acid can be single, double, triple, or quadruple stranded. See for example Bass (2001) Nature, 411, 428 429; Elbashir et al., (2001) Nature, 411, 494 498; and PCT Publication Nos. WO 00/44895, WO 01/36646, WO 99/32619, WO 00/01846, WO 01/29058, WO 99/07409, WO 00/44914; each of which are herein incorporated by reference in its entirety.

siRNA can be produced chemically or biologically, or can be expressed from a recombinant plasmid or viral vector (for example, see U.S. Pat. No. 7,294,504; U.S. Pat. No. 7,148,342; and U.S. Pat. No. 7,422,896; the entire disclosures of which are herein incorporated by reference). Exemplary methods for producing and testing dsRNA or siRNA molecules are described in U.S. Patent Application Publication No. 2002/0173478 to Gewirtz, and in U.S. Patent Application Publication No. 2007/0072204 to Hannon et al., the entire disclosures of which are herein incorporated by reference.

A MGES modulating compound can additionally be a short hairpin RNA (shRNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., 2002, Genes Dev, 16:948-58; McCaffrey et al., 2002, Nature, 418:38-9; McManus et al., 2002, RNA, 8:842-50; Yu et al., 2002, Proc Natl Acad Sci USA, 99:6047-52; each herein incorporated by reference in its entirety. Such hairpin RNAs are engineered in cells or in an animal to ensure continuous and stable suppression of a desired gene. It is known in the art that siRNAs can be produced by processing a hairpin RNA in the cell.

When a nucleic acid such as RNA or DNA is used that encodes a protein or peptide of the invention, it can be delivered into a cell in any of a variety of forms, including as naked plasmid or other DNA, formulated in liposomes, in an expression vector, which includes a viral vector (including RNA viruses and DNA viruses, including adenovirus, lentivirus, alphavirus, and adeno-associated virus), by biocompatible gels, via a pressure injection apparatus such as the Powderject™ system using RNA or DNA, or by any other convenient means. Again, the amount of nucleic acid needed to sequester an Id protein in the cytoplasm can be readily determined by those of skill in the art, which also can vary with the delivery formulation and mode and whether the nucleic acid is DNA or RNA. For example, see Manjunath et al., (2009) Adv Drug Deliv Rev. 61(9):732-45; Singer and Verma, (2008) Curr Gene Ther. 8(6):483-8; and Lundberg et al., (2008) Curr Gene Ther. 8(6):461-73; each herein incorporated by reference in its entirety.

A MGES modulating compound can also be a small molecule that binds to the MGES and disrupts its function, or conversely, enhances its function. Small molecules are a diverse group of synthetic and natural substances having low molecular weights. They can be isolated from natural sources (for example, plants, fungi, microbes and the like), are obtained commercially and/or available as libraries or collections, or synthesized. Candidate small molecules that modulate MGES can be identified via in silico screening or high-throughput (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries as described herein (Werner et al., (2006) Brief Funct. Genomic Proteomic 5(1):32-6; herein incorporated by reference in its entirety).

In some embodiments, the compound is selected from the group consisting of etoposide, 5-fluorouracil, Clostridium difficile Toxin B,

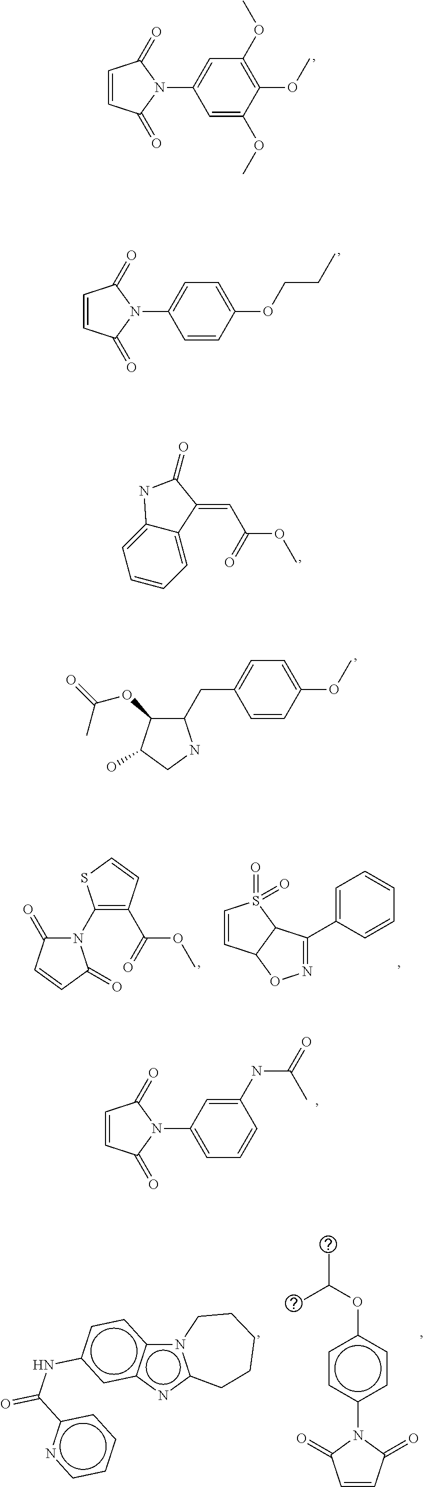

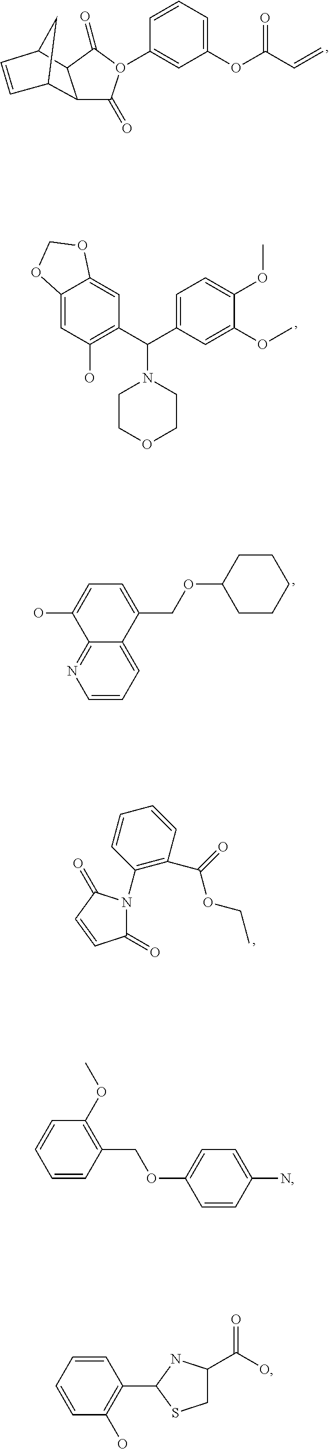

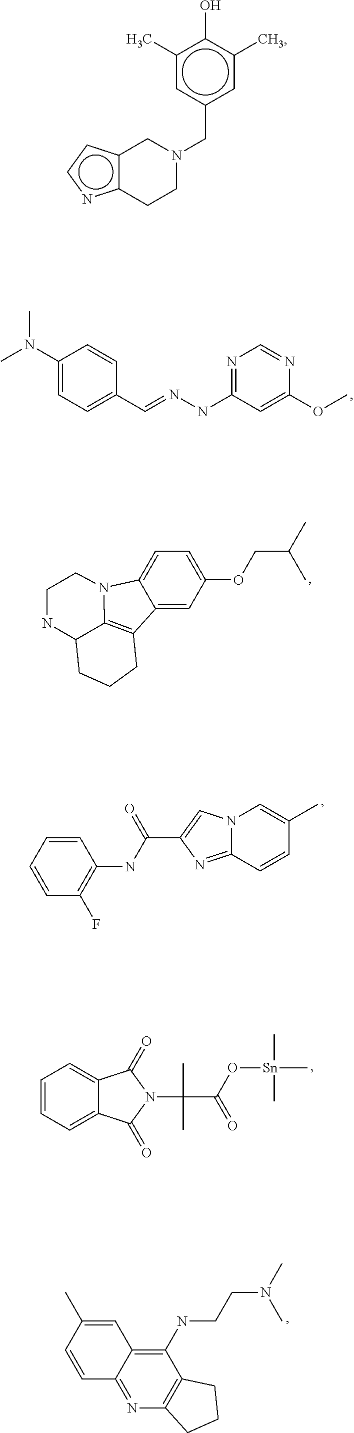

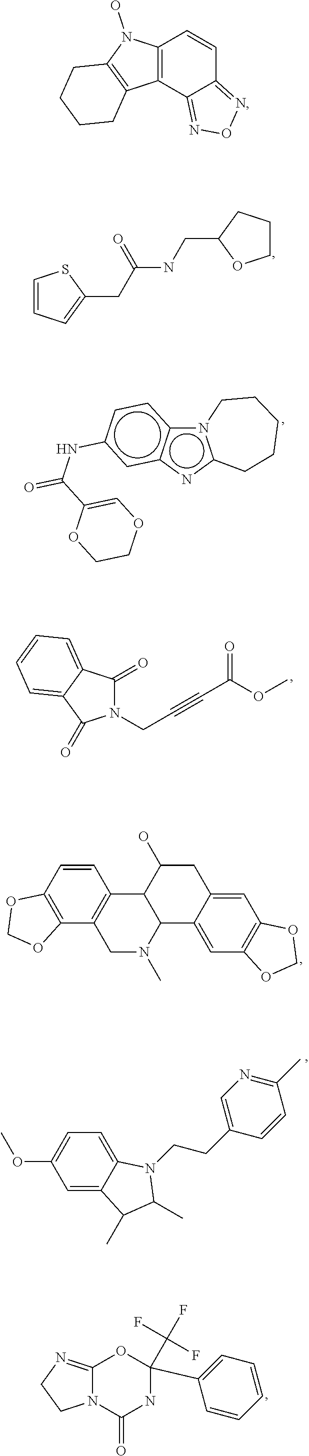

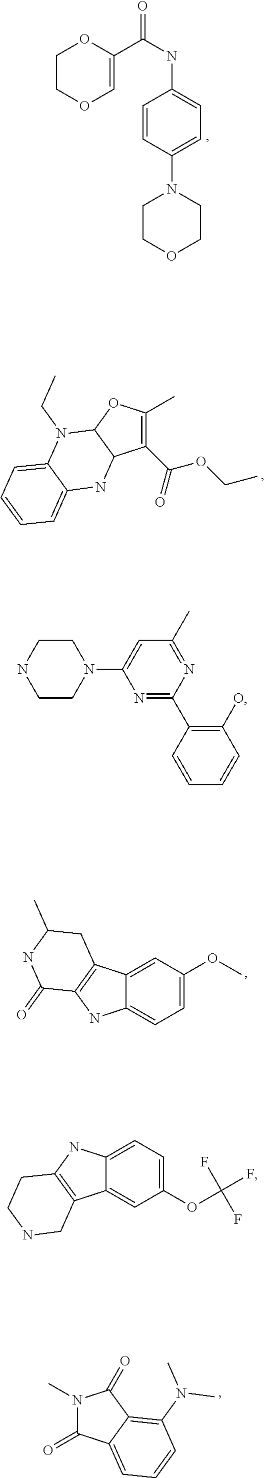

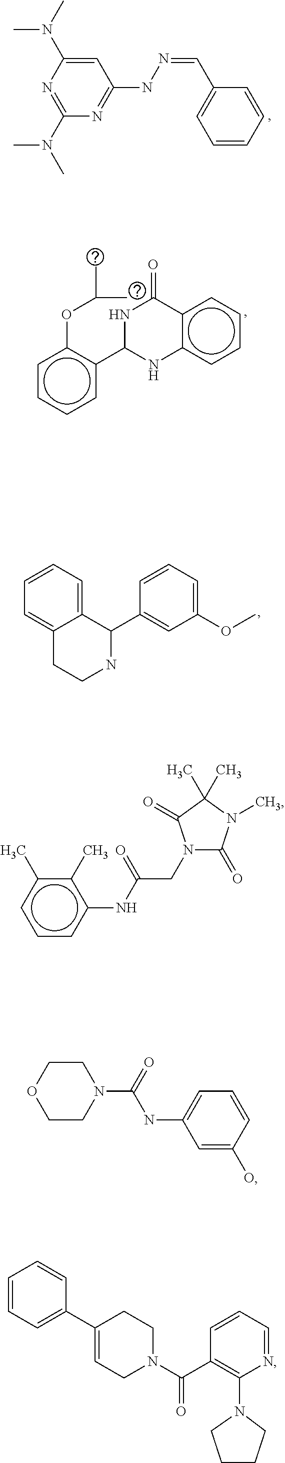

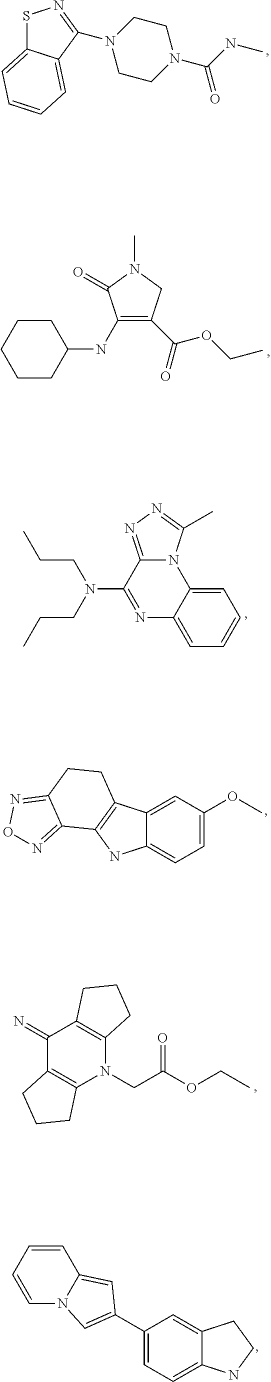

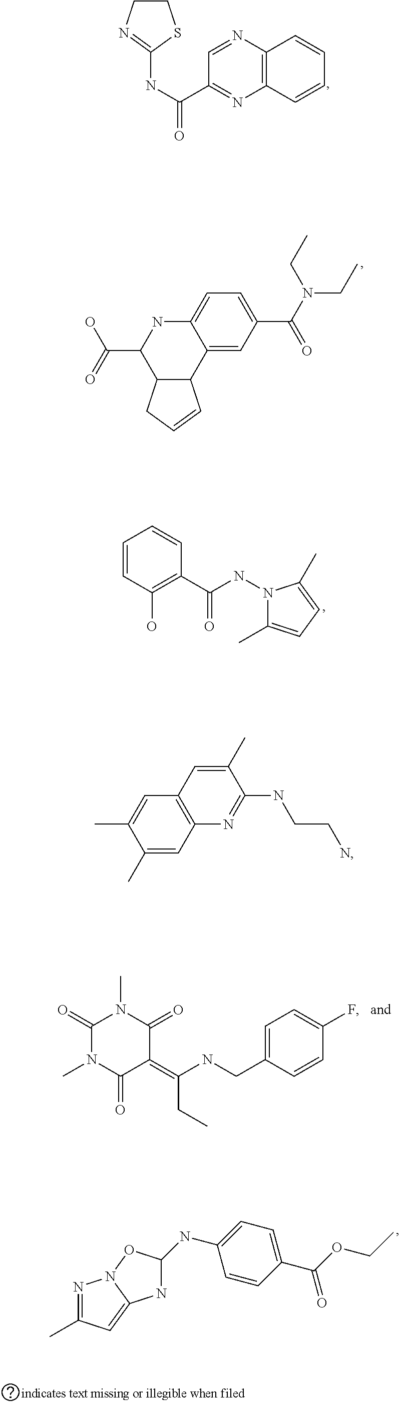

and pharmaceutically acceptable salts thereof.

In some embodiments, the compound is selected from the group consisting of 5-fluorouracil, Clostridium difficile Toxin B,

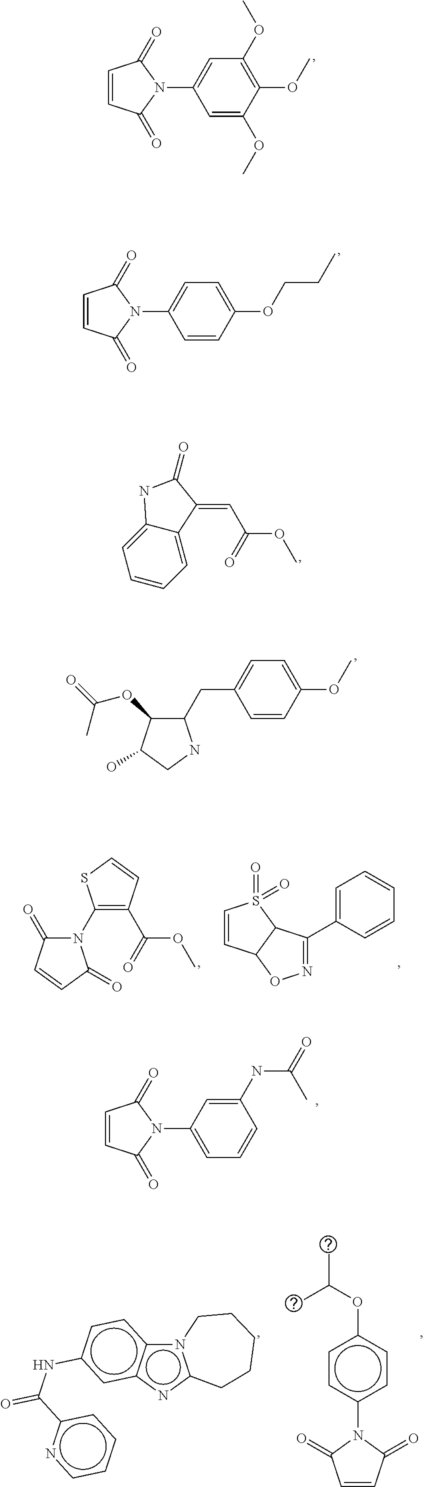

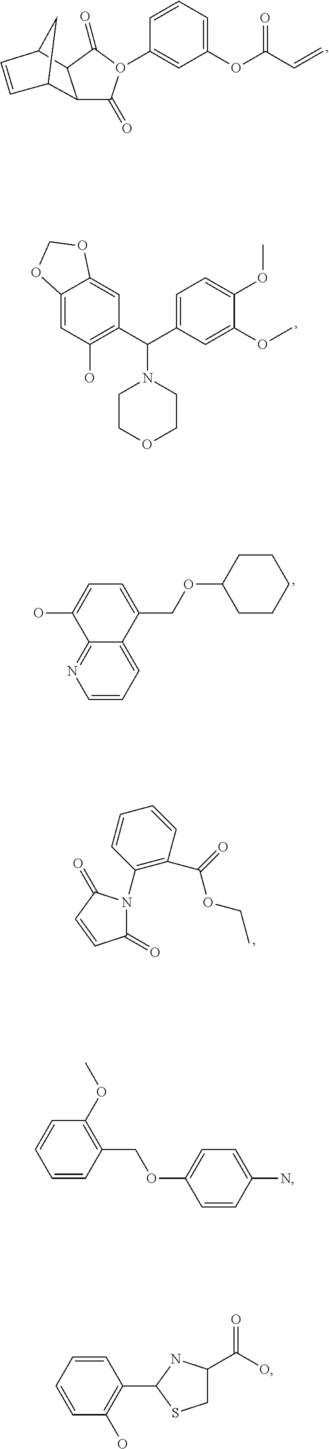

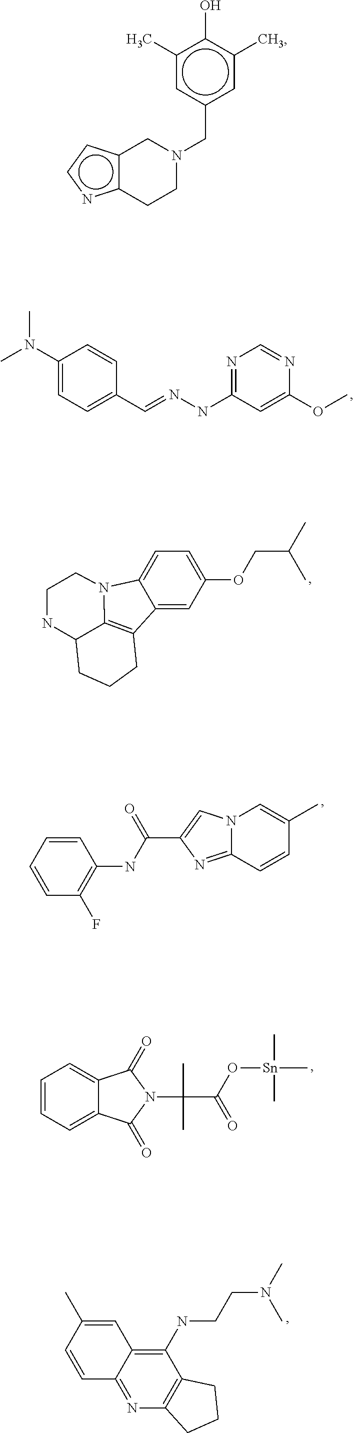

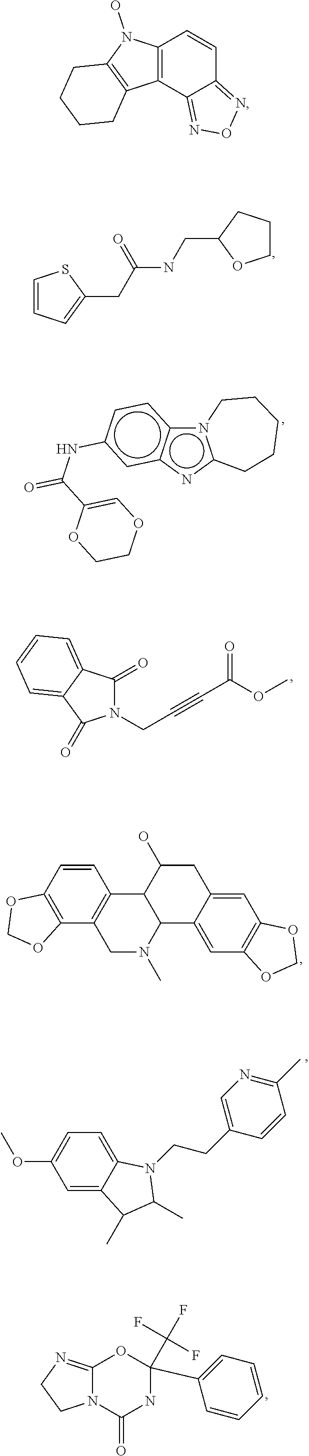

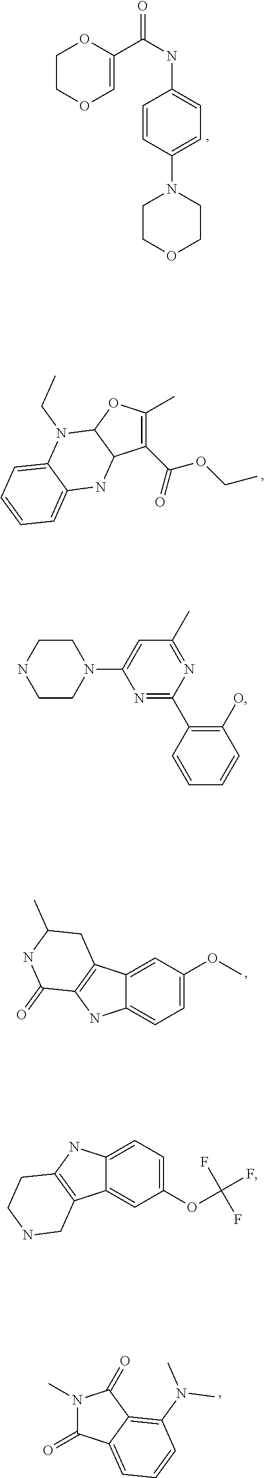

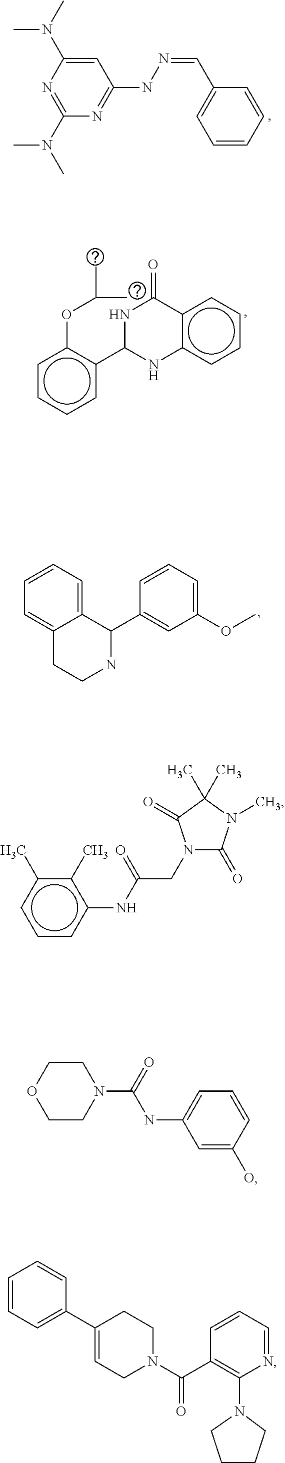

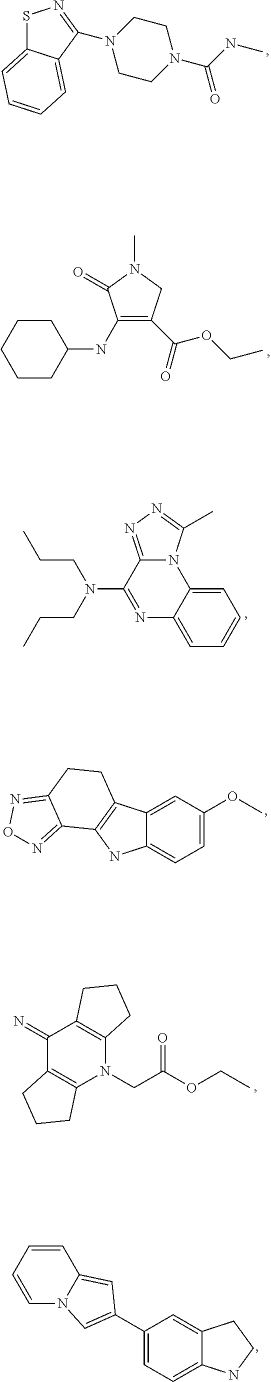

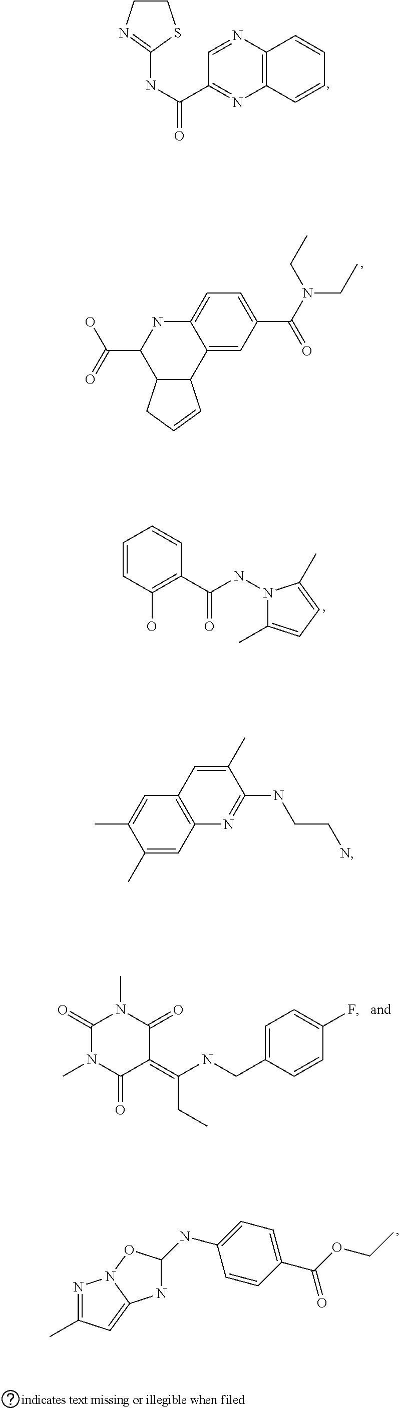

and pharmaceutically acceptable salts thereof.

In some embodiments, the compound is selected from the group consisting of Clostridium difficile Toxin B,

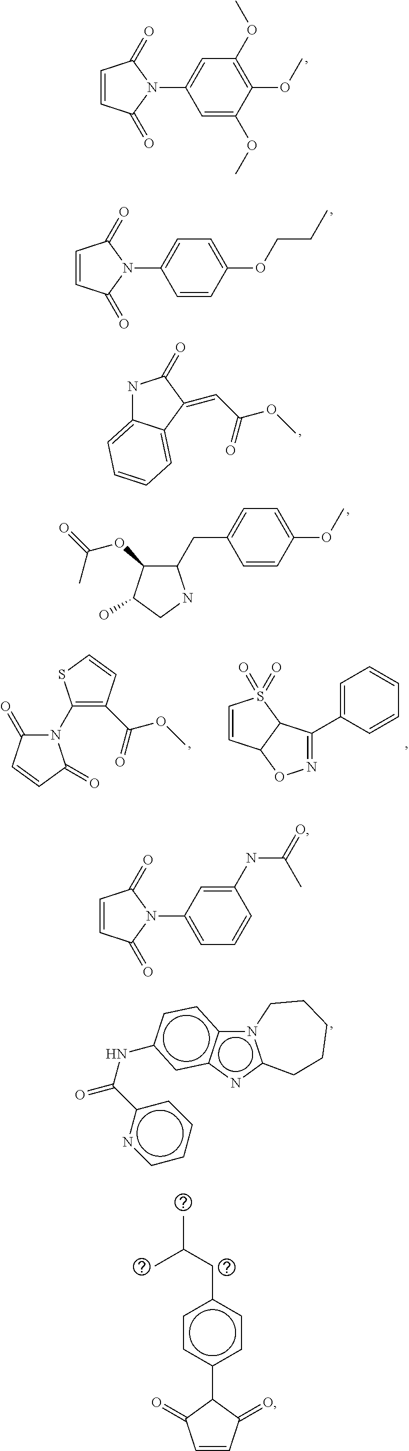

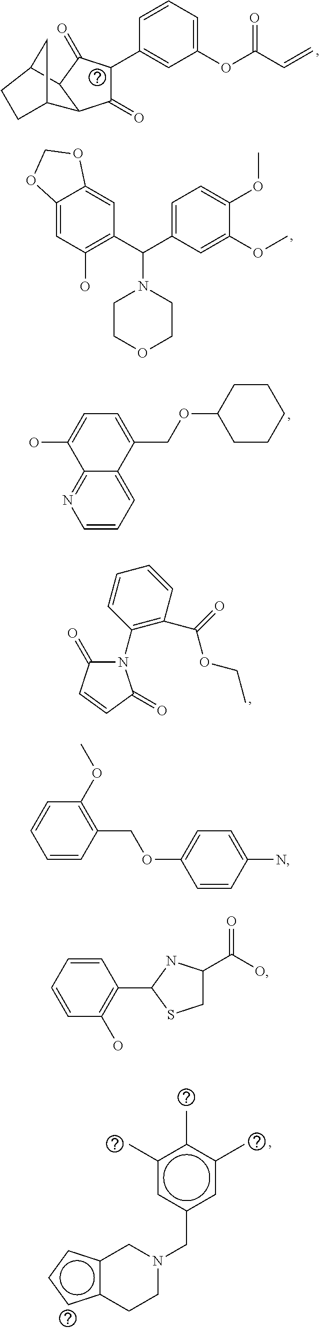

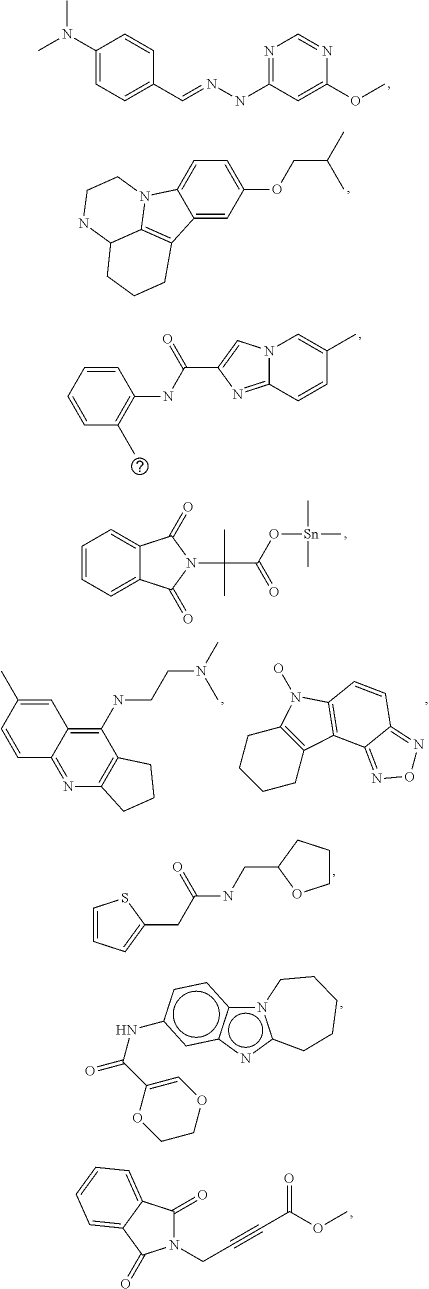

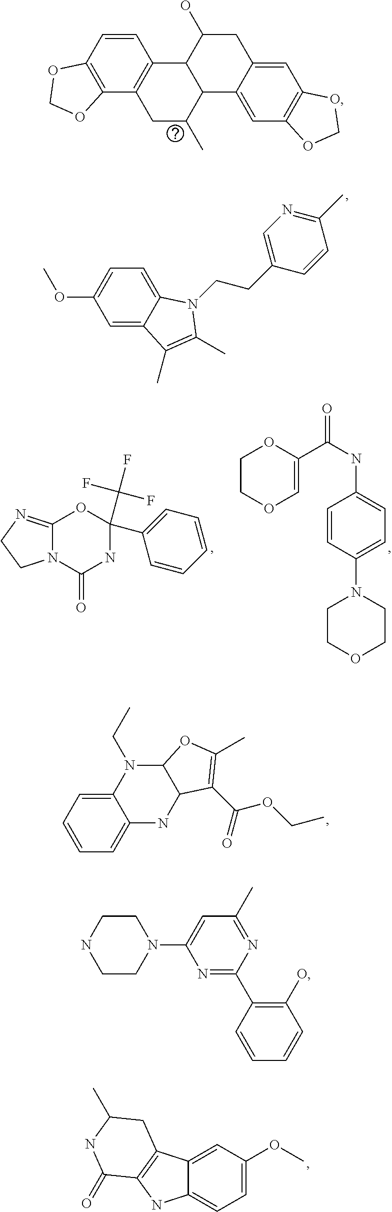

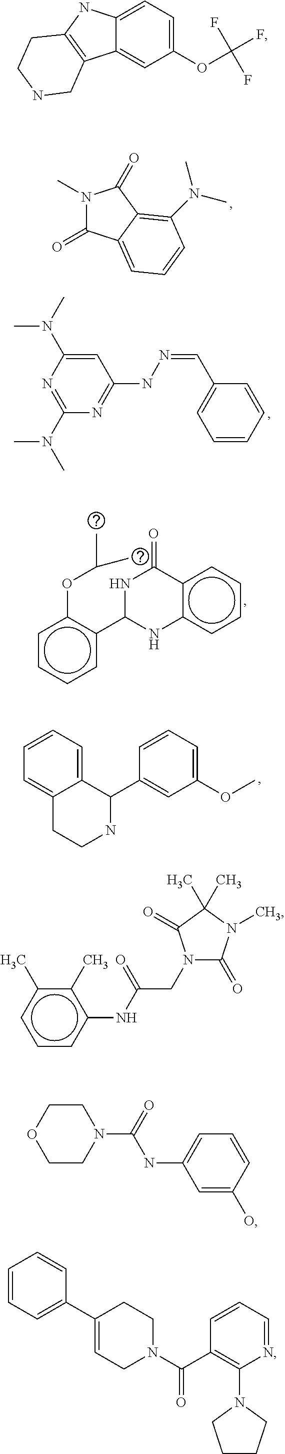

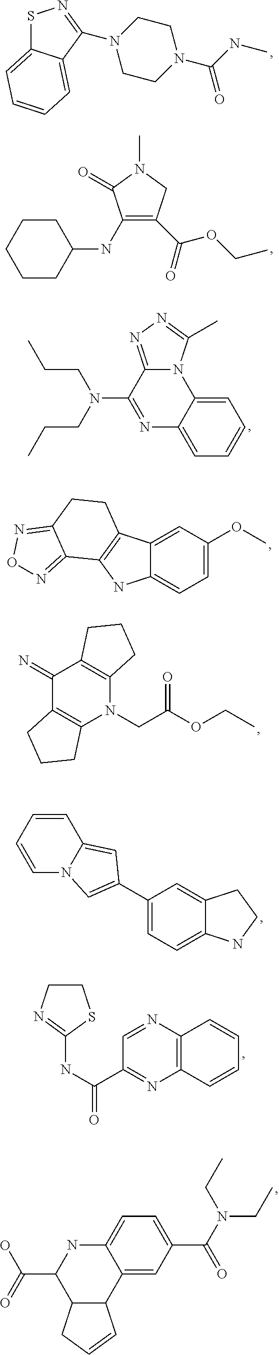

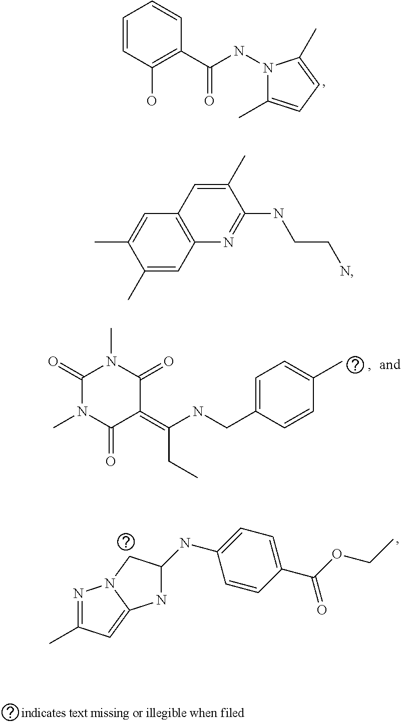

and pharmaceutically acceptable salts thereof.

In some embodiments, the compound is selected from the group consisting of

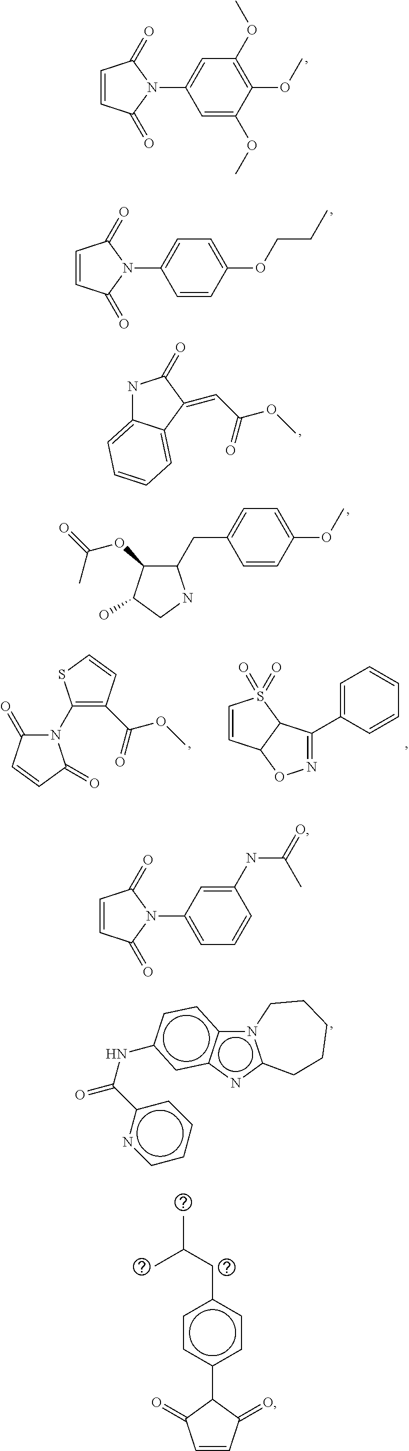

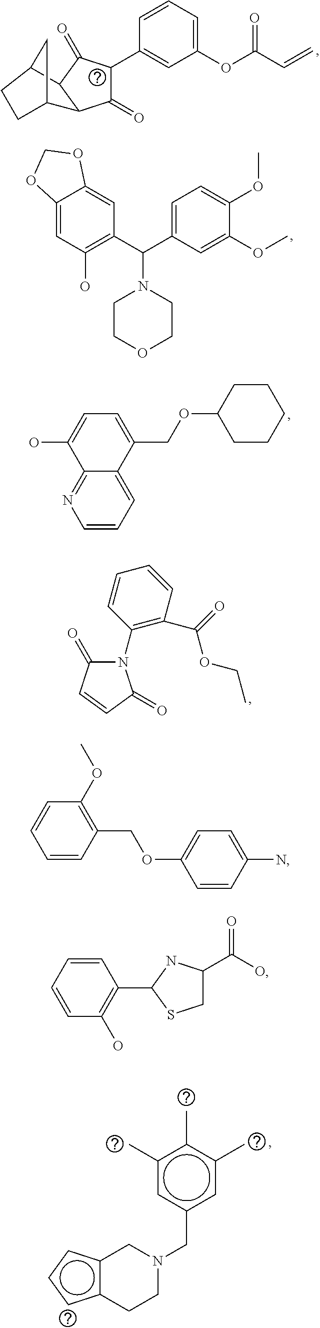

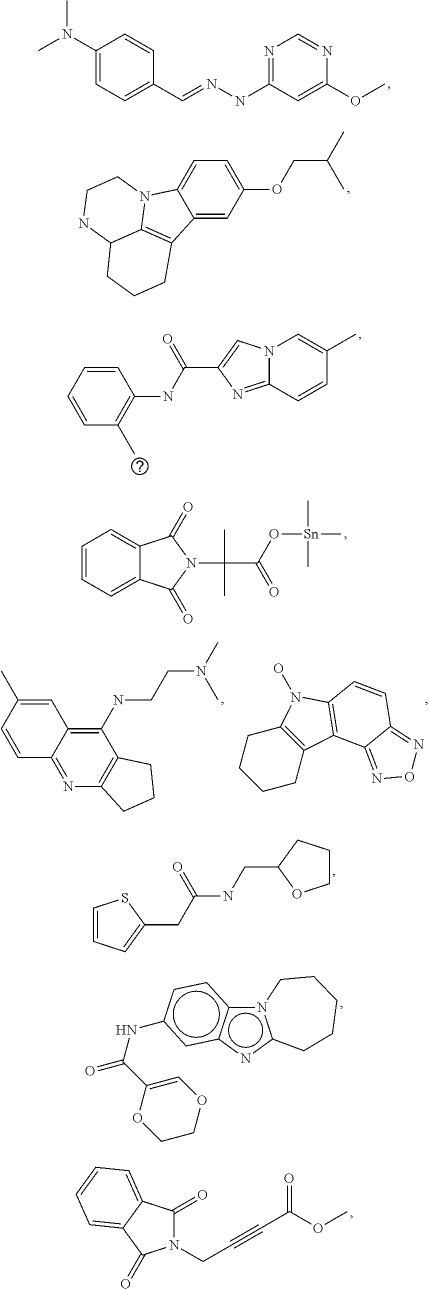

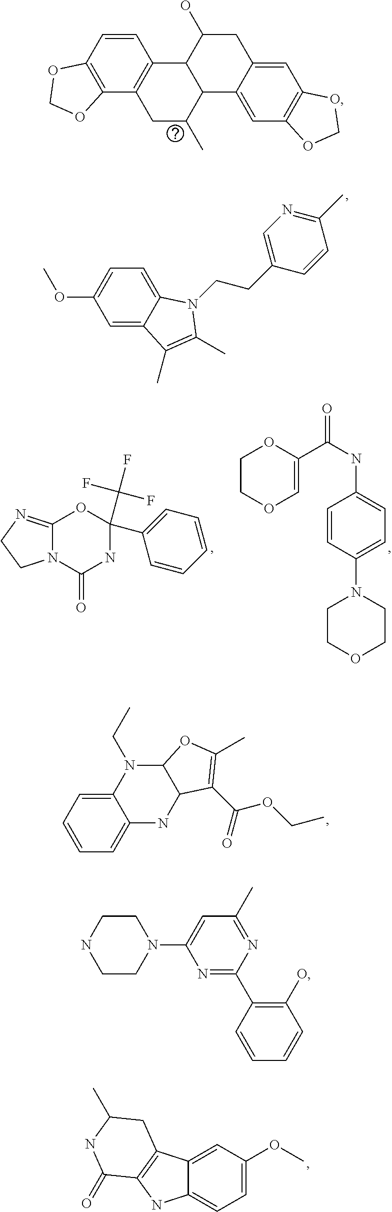

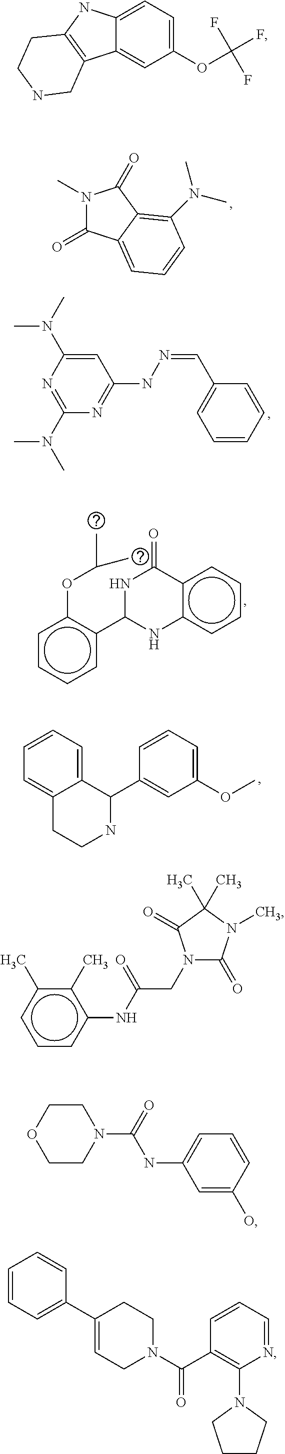

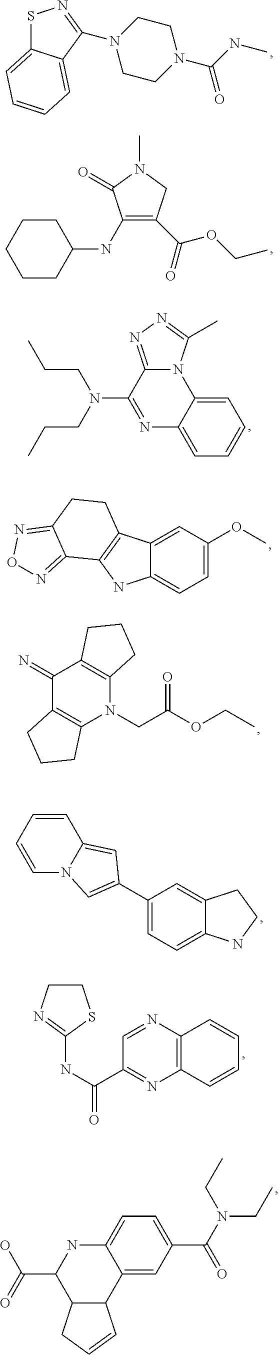

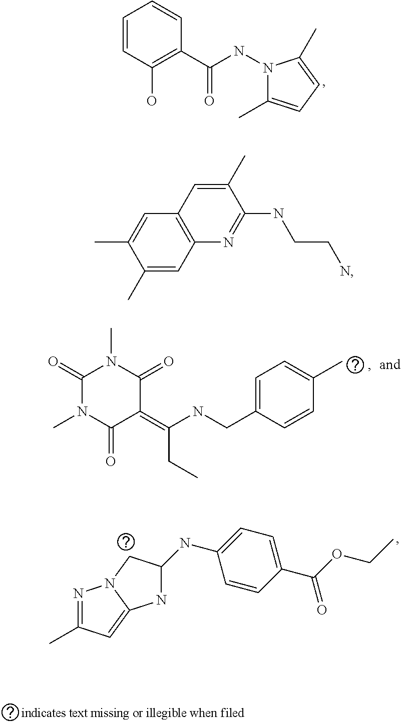

and pharmaceutically acceptable salts thereof.

In some embodiments, the compound is

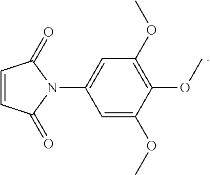 [SMILES: COc1cc(N2C(=O)C=CC2=O)cc(OC)c1OC]

In some embodiments, the compound is

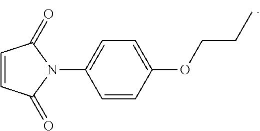 [SMILES: CCCOc1ccc(N2C(=O)C=CC2=O)cc1]

In some embodiments, the compound is

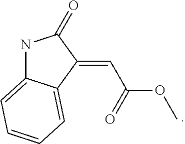

In some embodiments, the compound is

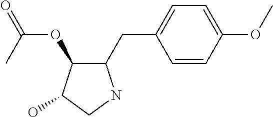 [SMILES: COc1ccc(CC2NC[C@H](O)[C@H]2OC(C)=O)cc1]

In some embodiments, the compound is

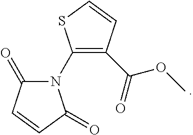 [SMILES: COC(=O)c1ccsc1N1C(=O)C=CC1=O]

In some embodiments, the compound is

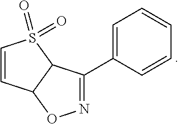 [SMILES: O=S1(=O)C=CC2ON=C(c3ccccc3)C21]

In some embodiments, the compound is

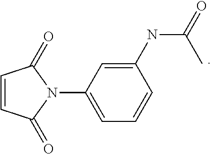 [SMILES: CC(=O)Nc1cccc(N2C(=O)C=CC2=O)c1]

In some embodiments, the compound is

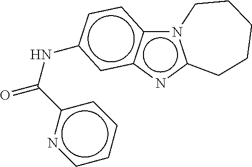

In some embodiments, the compound is

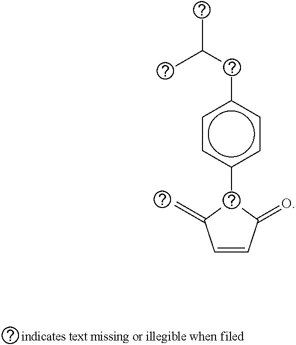

In some embodiments, the compound is

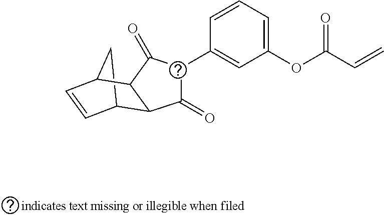 [SMILES: C=CC(=O)Oc1cccc(C2C(=O)C3C4C=CC(C4)C3C2=O)c1]

In some embodiments, the compound is

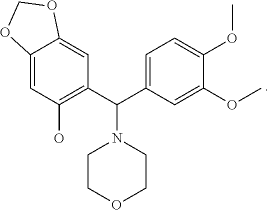 [SMILES: COc1ccc(C(c2cc3c(cc2O)OCO3)N2CCOCC2)cc1OC]

In some embodiments, the compound is

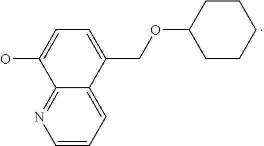 [SMILES: Oc1ccc(COC2CCCCC2)c2cccnc12]

In some embodiments, the compound is

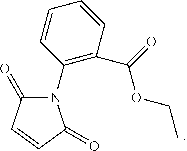 [SMILES: CCOC(=O)c1ccccc1N1C(=O)C=CC1=O]

In some embodiments, the compound is

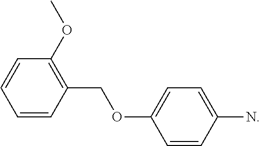

In some embodiments, the compound is

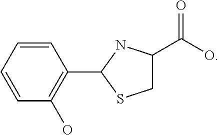

In some embodiments, the compound is

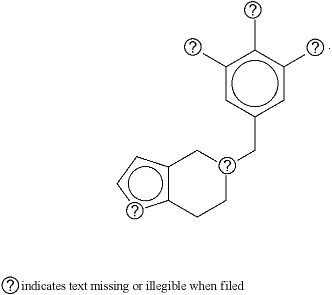

In some embodiments, the compound is

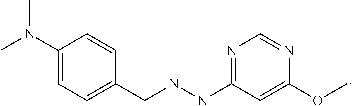

In some embodiments, the compound is

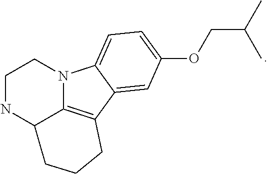 [SMILES: CC(C)COc1ccc2c(c1)c1c3n2CCNC3CCC1]

In some embodiments, the compound is

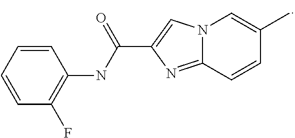 [SMILES: Cc1ccc2nc(C(=O)Nc3ccccc3F)cn2c1]

In some embodiments, the compound is

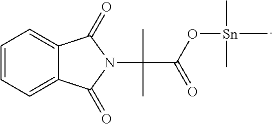 [SMILES: CC(C)(C(=O)[O][Sn]([CH3])([CH3])[CH3])N1C(=O)c2ccccc2C1=O]

In some embodiments, the compound is

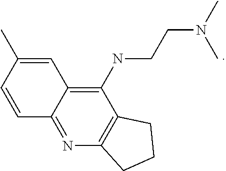 [SMILES: Cc1ccc2nc3c(c(NCCN(C)C)c2c1)CCC3]

In some embodiments, the compound is

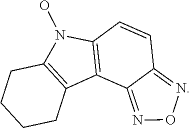 [SMILES: On1c2c(c3c4nonc4ccc31)CCCC2]

In some embodiments, the compound is

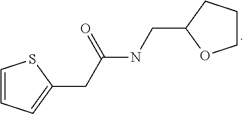 [SMILES: O=C(Cc1cccs1)NCC1CCCO1]

In some embodiments, the compound is

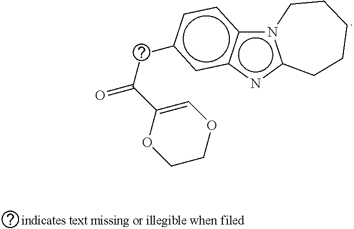

In some embodiments, the compound is

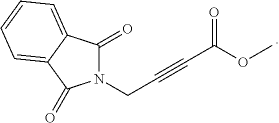 [SMILES: COC(=O)C#CCN1C(=O)c2ccccc2C1=O]

In some embodiments, the compound is

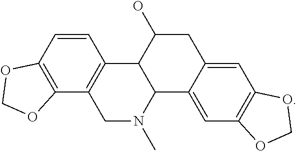 [SMILES: CN1Cc2c(ccc3c2OCO3)C2C(O)Cc3cc4c(cc3C21)OCO4]

In some embodiments, the compound is

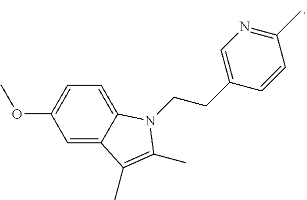

In some embodiments, the compound is

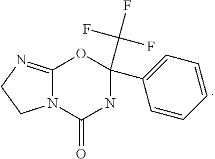 [SMILES: O=C1NC(c2ccccc2)(C(F)(F)F)OC2=NCCN12]

In some embodiments, the compound is

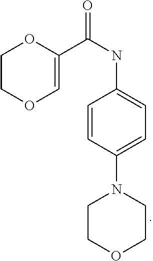 [SMILES: O=C(Nc1ccc(N2CCOCC2)cc1)C1=COCCO1]

In some embodiments, the compound is

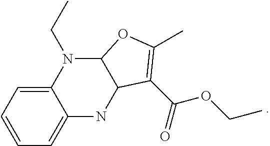 [SMILES: CCOC(=O)C1=C(C)OC2C1Nc1ccccc1N2CC]

In some embodiments, the compound is

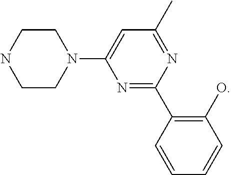

In some embodiments, the compound is

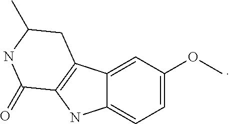 [SMILES: COc1ccc2[nH]c3c(c2c1)CC(C)NC3=O]

In some embodiments, the compound is

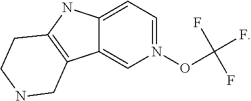

In some embodiments, the compound is

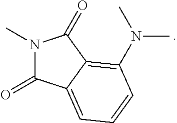

In some embodiments, the compound is

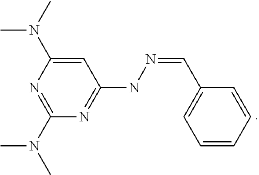 [SMILES: CN(C)c1cc(N/N=C\c2ccccc2)nc(N(C)C)n1]

In some embodiments, the compound is

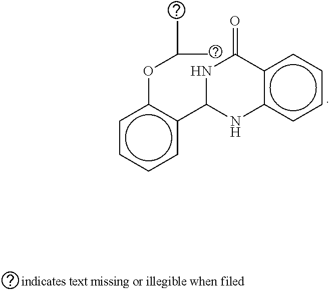

In some embodiments, the compound is

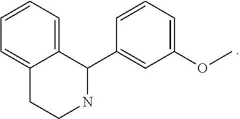 [SMILES: COc1cccc(C2NCCc3ccccc32)c1]

In some embodiments, the compound is

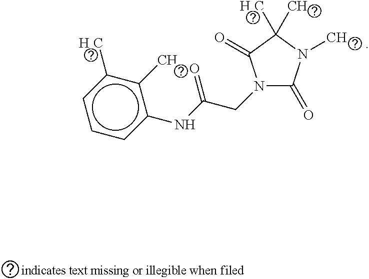

In some embodiments, the compound is

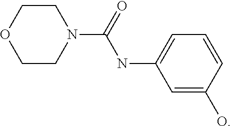

In some embodiments, the compound is

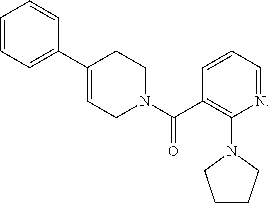 [SMILES: O=C(c1cccnc1N1CCCC1)N1CC=C(c2ccccc2)CC1]

In some embodiments, the compound is

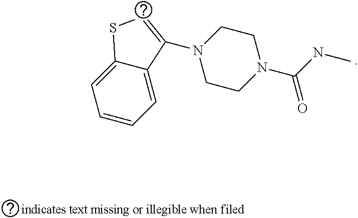 [SMILES: CNC(=O)N1CCN(c2csc3ccccc23)CC1]

In some embodiments, the compound is

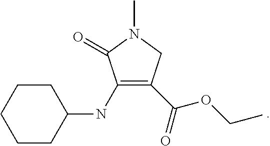 [SMILES: CCOC(=O)C1=C(NC2CCCCC2)C(=O)N(C)C1]

In some embodiments, the compound is.

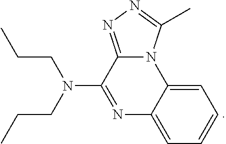 [SMILES: CCCN(CCC)c1nc2ccccc2n2c(C)nnc12]

In some embodiments, the compound is

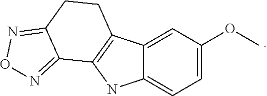 [SMILES: COc1ccc2[nH]c3c(c2c1)CCc1nonc1-3]

In some embodiments, the compound is

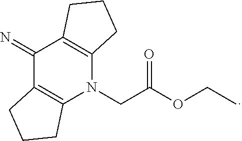 [SMILES: CCOC(=O)Cn1c2c(c(=N)c3c1CCC3)CCC2]

In some embodiments, the compound is

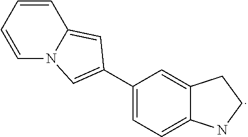 [SMILES: c1ccn2cc(-c3ccc4c(c3)CCN4)cc2c1]

In some embodiments, the compound is

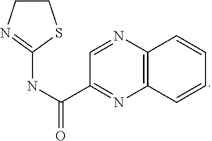 [SMILES: O=C(NC1=NCCS1)c1cnc2ccccc2n1]

In some embodiments, the compound is

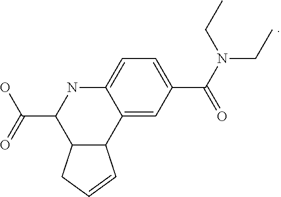 [SMILES: CCN(CC)C(=O)c1ccc2c(c1)C1C=CCC1C(C(=O)O)N2]

In some embodiments, the compound is

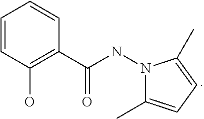 [SMILES: Cc1ccc(C)n1NC(=O)c1ccccc1O]

In some embodiments, the compound is

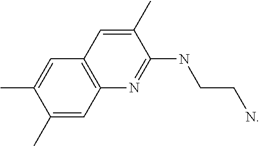

In some embodiments, the compound is

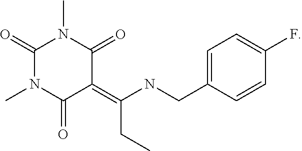 [SMILES: CCC(NCc1ccc(F)cc1)=C1C(=O)N(C)C(=O)N(C)C1=O]

In some embodiments, the compound is

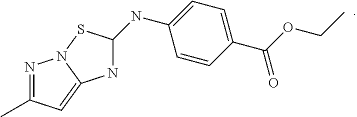 [SMILES: CCOC(=O)c1ccc(NC2Nc3cc(C)nn3S2)cc1]

In some embodiments, the compound is etoposide, 5-fluorouracil, or Clostridium difficile Toxin B. In some embodiments, the compound is etoposide. In some embodiments, the compound is 5-fluorouracil. In some embodiments, the compound is Clostridium difficile Toxin B.

Test compounds, such as MGES modulating compounds, can be screened from large libraries of synthetic or natural compounds (see Wang et al., (2007) Curr Med Chem, 14(2):133-55; Mannhold (2006) Curr Top Med Chem, 6 (10):1031-47; and Hensen (2006) Curr Med Chem 13(4):361-76; each herein incorporated by reference in its entirety). Various methods are currently used for random and directed synthesis of saccharide, peptide, and nucleic acid based compounds. Synthetic compound libraries are commercially available from Maybridge Chemical Co. (Trevillet, Cornwall, UK), AMRI (Albany, N.Y.), ChemBridge (San Diego, Calif.), and MicroSource (Gaylordsville, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60; herein incorporated by reference in its entirety). Many of these compounds are available from commercial source vendors such as, for example, Asinex, IBS, ChemBridge, Enamine, Life, TimTech, and Sigma-Aldrich.

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. Libraries are also meant to include for example but are not limited to peptide-on-plasmid libraries, polysome libraries, aptamer libraries, synthetic peptide libraries, synthetic small molecule libraries, neurotransmitter libraries, and chemical libraries. The libraries can also comprise cyclic carbon or heterocyclic structure and/or aromatic or polyaromatic structures substituted with one or more of the functional groups described herein.

Small molecule combinatorial libraries can also be generated and screened. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, each hereby incorporated by reference in its entirety.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/20242, dated Oct. 14, 1993; and Brenner et al., (1992) Proc. Natl. Acad. Sci. USA 89:5381-5383. Examples of phage display libraries are described in Scott et al., (1990) Science 249:386-390; Devlin et al., (1990) Science, 249:404-406; Christian, et al., (1992) J. Mol. Biol. 227:711-718; Lenstra, (1992) J. Immunol. Meth. 152:149-157; Kay et al., (1993) Gene 128:59-65; and PCT Publication No. WO 94/18318. In vitro translation-based libraries include but are not limited to those described in PCT Publication No. WO 91/05058; and Mattheakis et al., (1994) Proc. Natl. Acad. Sci. USA 91:9022-9026. Each of the foregoing publications are incorporated by reference herein in their entireties.

Computer modeling and searching technologies permit the identification of compounds, or the improvement of already identified compounds, that can modulate MGES expression or activity. Having identified such a compound or composition, the active sites or regions of a MGES molecule can be subsequently identified via examining the sites as to which the compounds bind. These active sites can be ligand binding sites and can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:571-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1993) Science 263:671-673; and PCT Pub. WO 94/18318. Each of the foregoing publications are incorporated by reference herein in their entireties.

The three dimensional geometric structure of an active site, for example that of a MGES polypeptide can be determined by known methods in the art, such as X-ray crystallography, which can determine a complete molecular structure. Solid or liquid phase NMR can be used to determine certain intramolecular distances. Any other experimental method of structure determination can be used to obtain partial or complete geometric structures. The geometric structures can be measured with a complexed ligand, natural or artificial, which can increase the accuracy of the active site structure determined. Potential MGES modulating compounds can also be identified using the X-ray coordinates of another MGES transcription factor that is similar in structure to a MGES (such as, Stat3, C/EBPβ, C/EBPδ, RunX1, FosL2, bHLH-B2, ZNF238). In some embodiments, a compound that binds to a P2RY5 protein can be identified via: (1) providing an electronic library of test compounds; (2) providing atomic coordinates for at least 20 amino acid residues for the binding pocket of a MGES protein, wherein the coordinates have a root mean square deviation therefrom, with respect to at least 50% of Ca atoms, of not greater than about 5 Å, in a computer readable format; (3) converting the atomic coordinates into electrical signals readable by a computer processor to generate a three dimensional model of the rhodopsin protein, which is similar to the MGES protein; (4) performing a data processing method, wherein electronic test compounds from the library are superimposed upon the three dimensional model of the protein; and determining which test compound fits into the binding pocket of the three dimensional model, thereby identifying which compound binds to a MGES (e.g., Stat3, C/EBPβ, C/EBPδ, RunX1, FosL2, bHLH-B2, ZNF238).

Methods for predicting the effect on protein conformation of a change in protein sequence, are known in the art, and the skilled artisan can thus design a variant which functions as an antagonist according to known methods. One example of such a method is described by Dahiyat and Mayo in Science (1997) 278:82 87; herein incorporated by reference in its entirety, which describes the design of proteins de novo. The method can be applied to a known protein to vary only a portion of the polypeptide sequence. Similarly, Blake (U.S. Pat. No. 5,565,325; herein incorporated by reference iri its entirety.) teaches the use of known ligand structures to predict and synthesize variants with similar or modified function.

Other methods for preparing or identifying peptides that bind to a target are known in the art. Molecular imprinting, for instance, can be used for the de novo construction of macromolecular structures such as peptides that bind to a molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Mosbach, (1994) Trends in Biochem. Sci., 19(9); and Wulff, G., in Polymeric Reagents and Catalysts (Ford, W. T., Ed.) ACS Symposium Series No. 308, pp 186-230, American Chemical Society (1986); each herein incorporated by reference in its entirety. One method for preparing mimics of a MGES modulating compound involves the steps of: (i) polymerization of functional monomers around a known substrate (the template) that exhibits a desired activity; (ii) removal of the template molecule; and then (iii) polymerization of a second class of monomers in, the void left by the template, to provide a new molecule which exhibits one or more desired properties which are similar to that of the template. In addition to preparing peptides in this manner other binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are prepared by the free radical polymerization of functional monomers, resulting in a compound with a nonbiodegradable backbone. Other methods for designing such molecules include for example drug design based on structure activity relationships, which require the synthesis and evaluation of a number of compounds and molecular modeling.

MGES modulating compounds of the invention can be incorporated into pharmaceutical compositions suitable for administration, for example in combination with a pharmaceutically acceptable carrier. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones.

In some embodiments, the composition comprises a compound selected from the group consisting of etoposide, 5-fluorouracil, Clostridium difficile Toxin B,

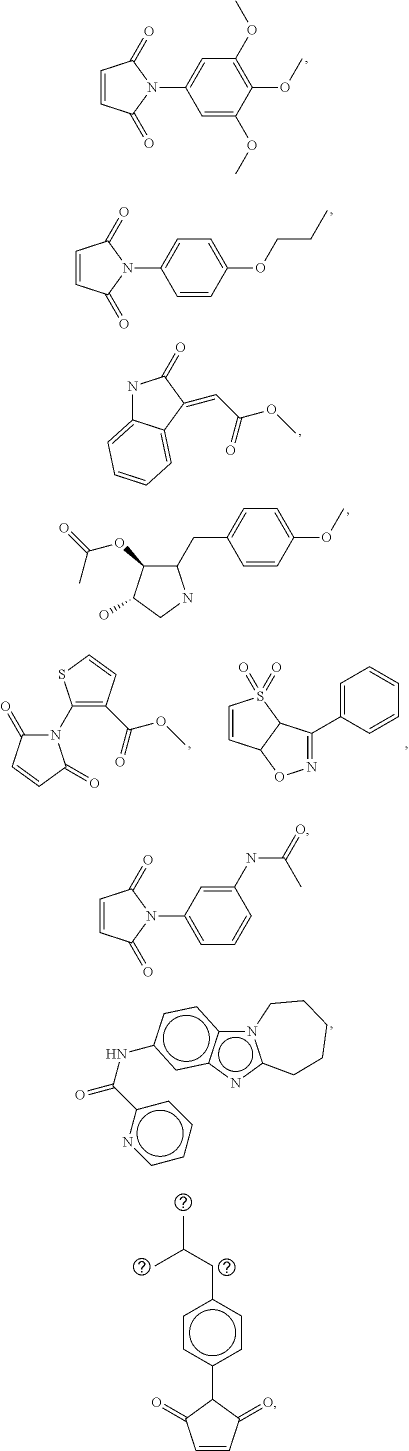

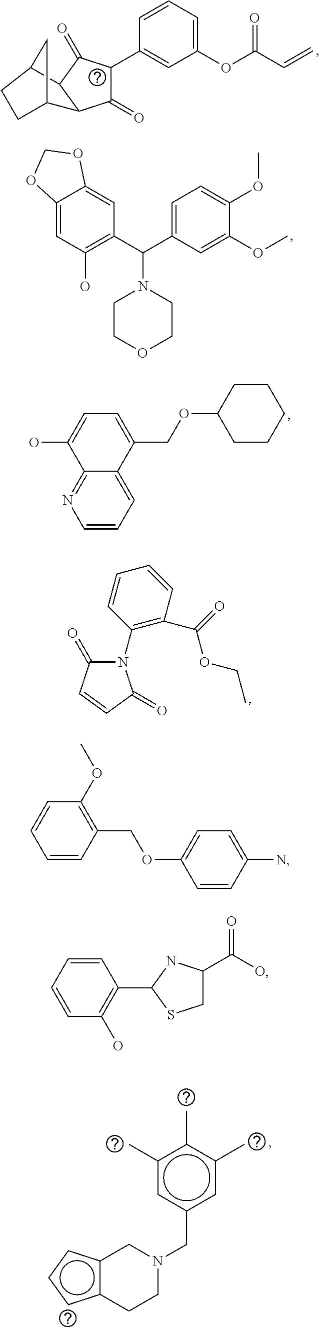

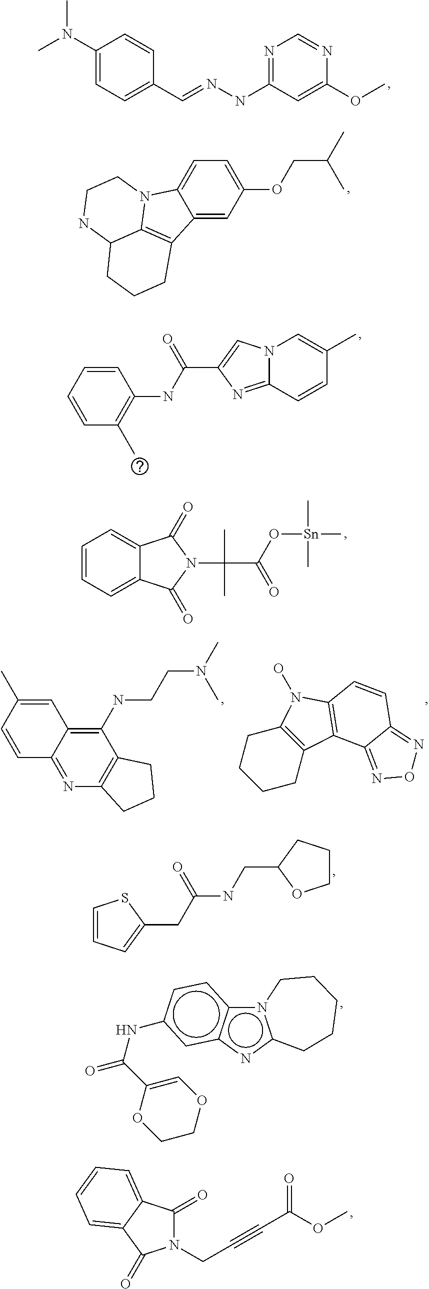

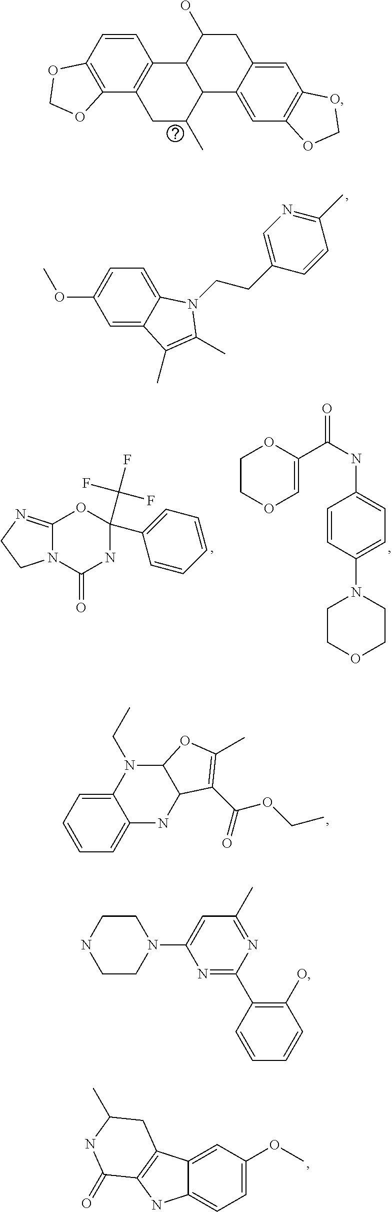

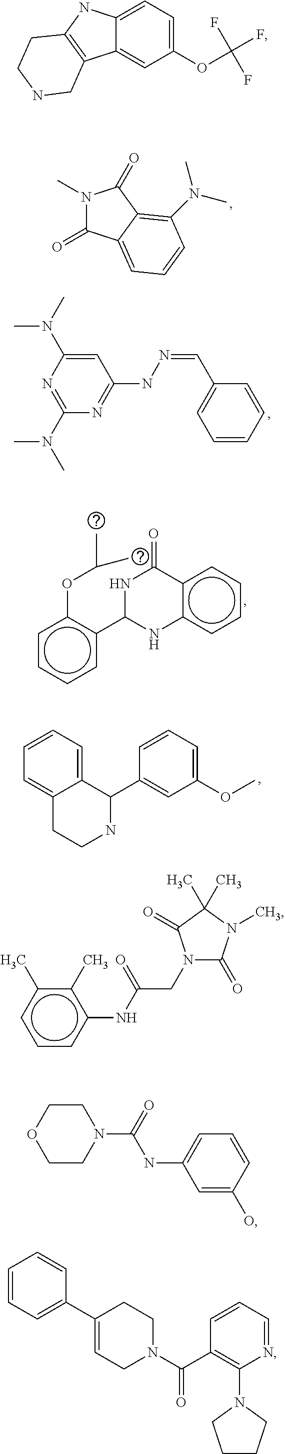

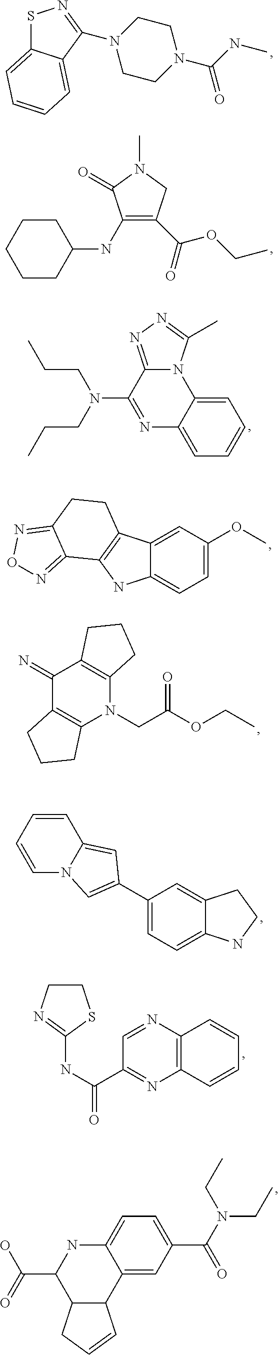

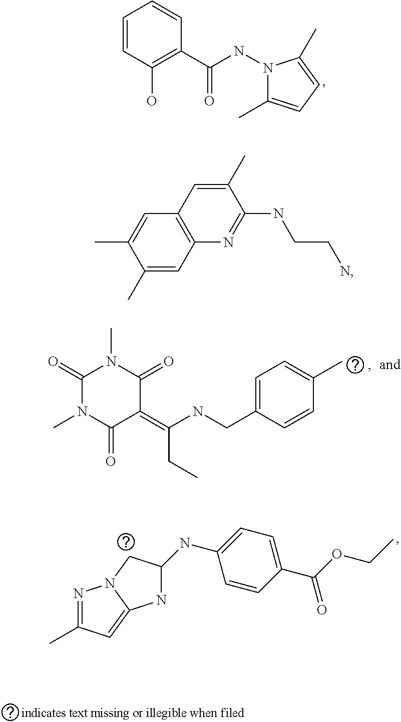

and pharmaceutically acceptable salts thereof.

In some embodiments, the composition comprises a compound selected from the group consisting of 5-fluorouracil, Clostridium difficile Toxin B,

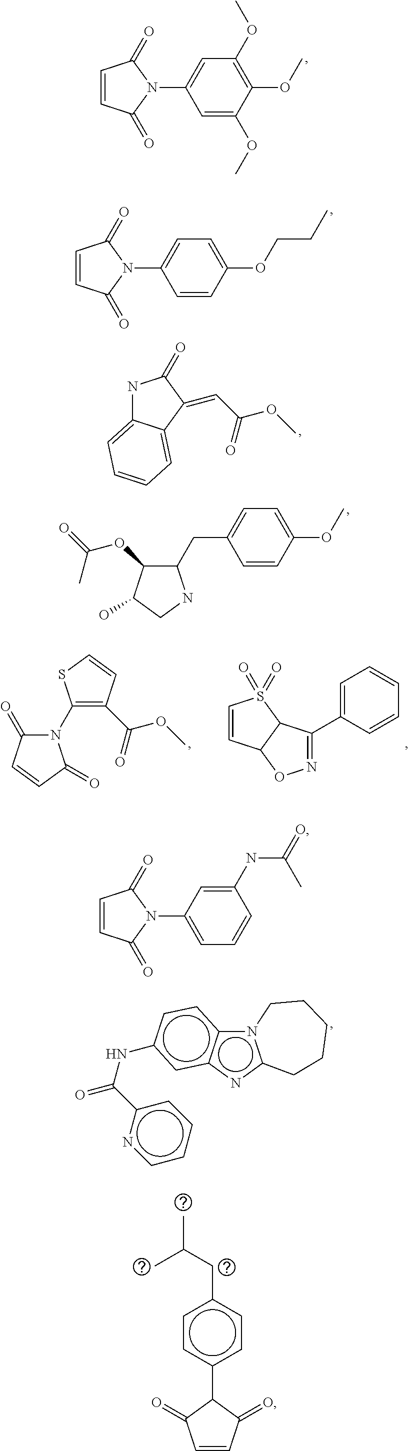

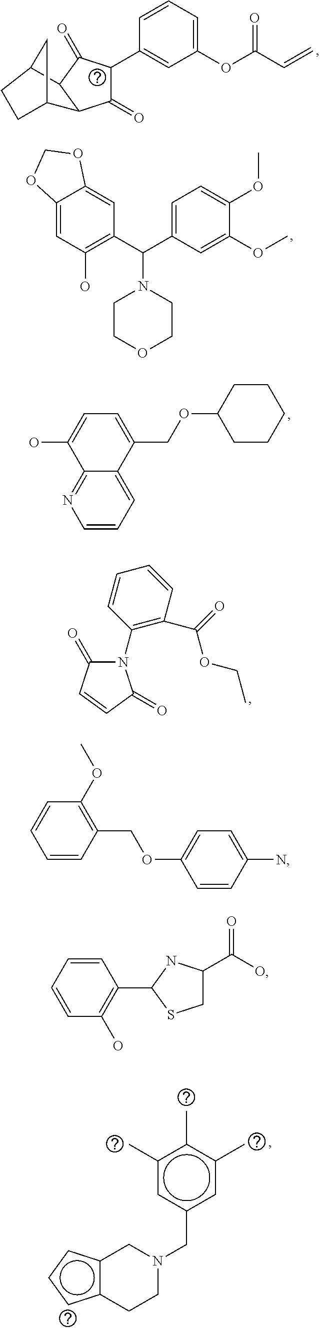

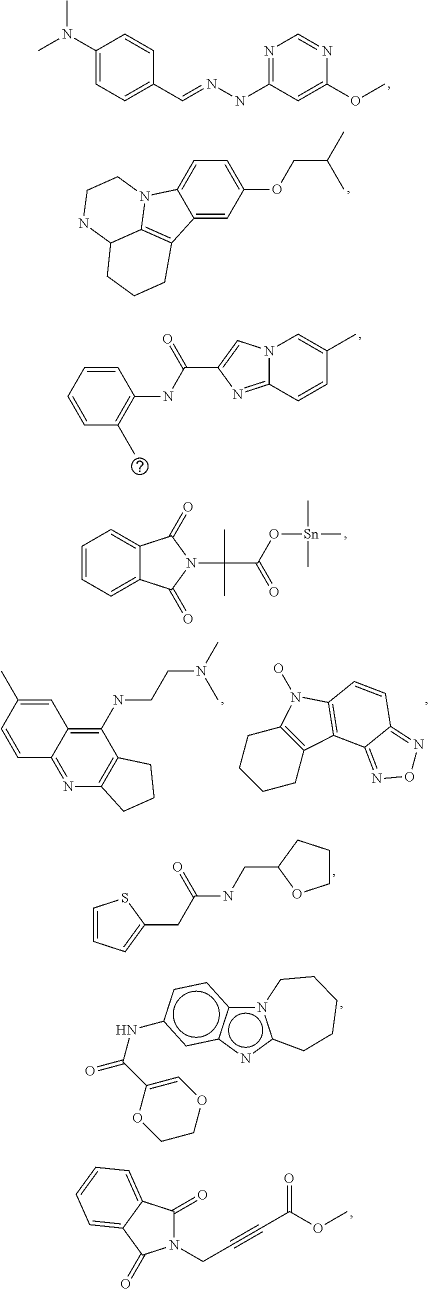

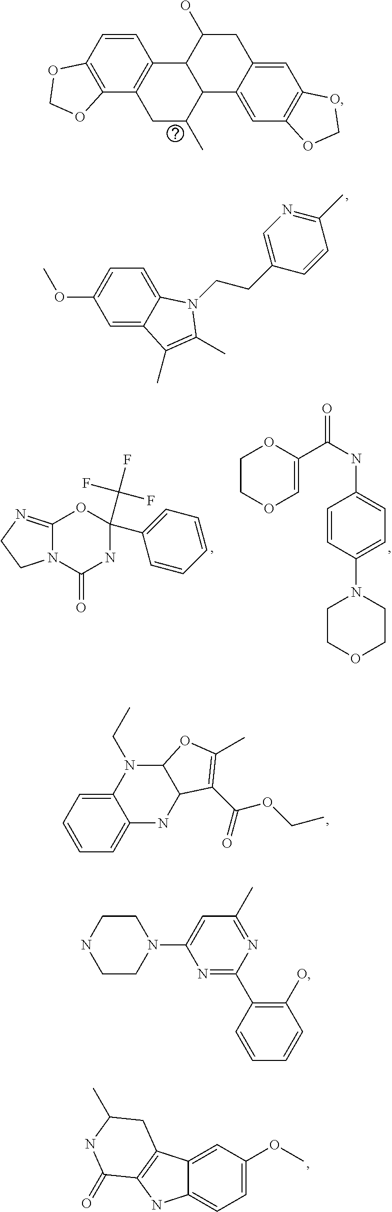

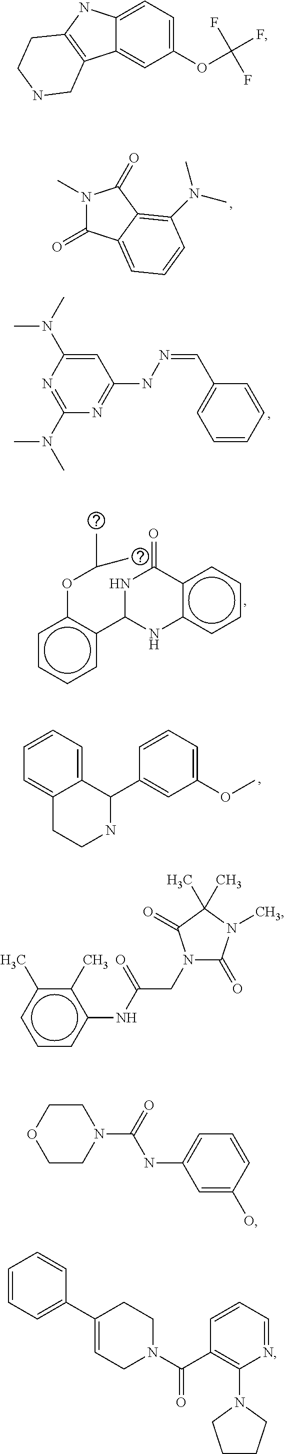

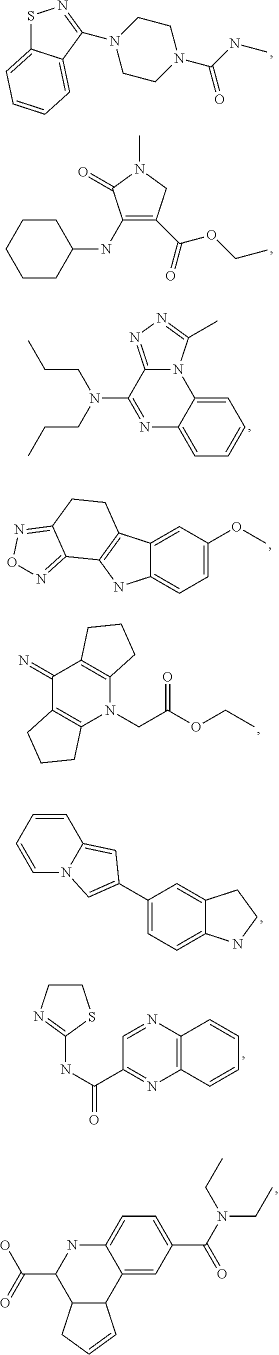

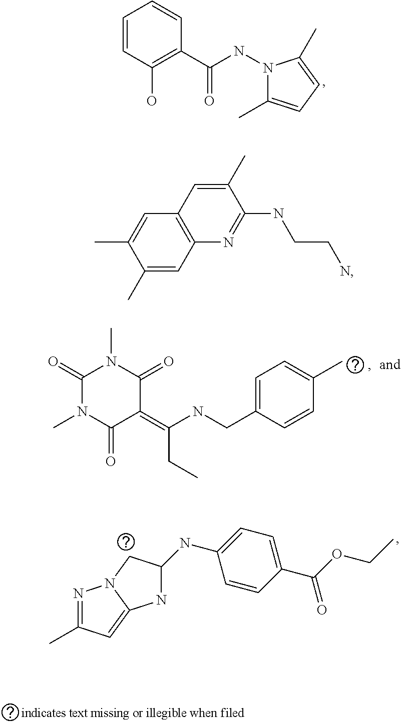

and pharmaceutically acceptable salts thereof.

In some embodiments, the composition comprises a compound selected from the group consisting of Clostridium difficile Toxin B,

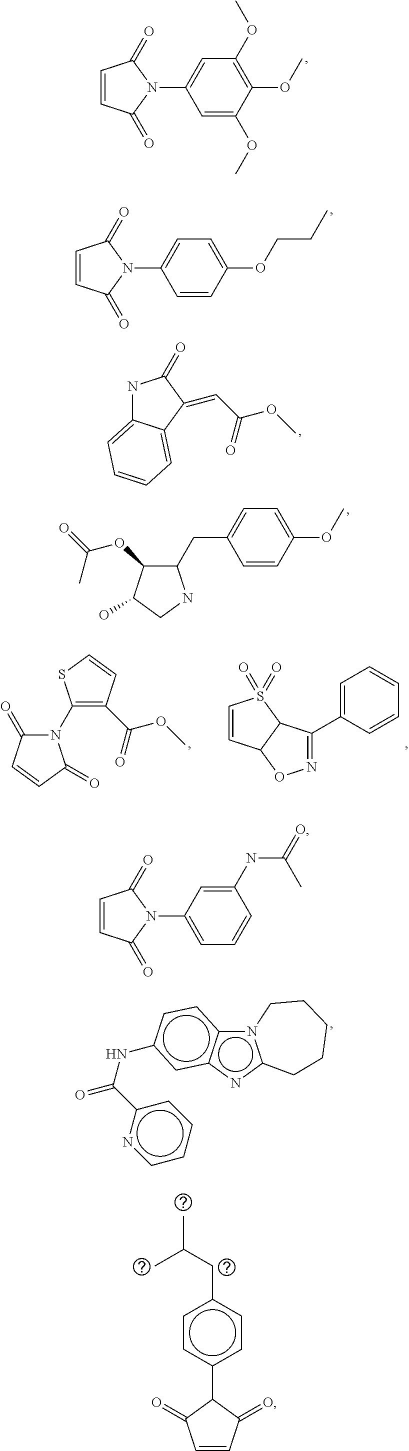

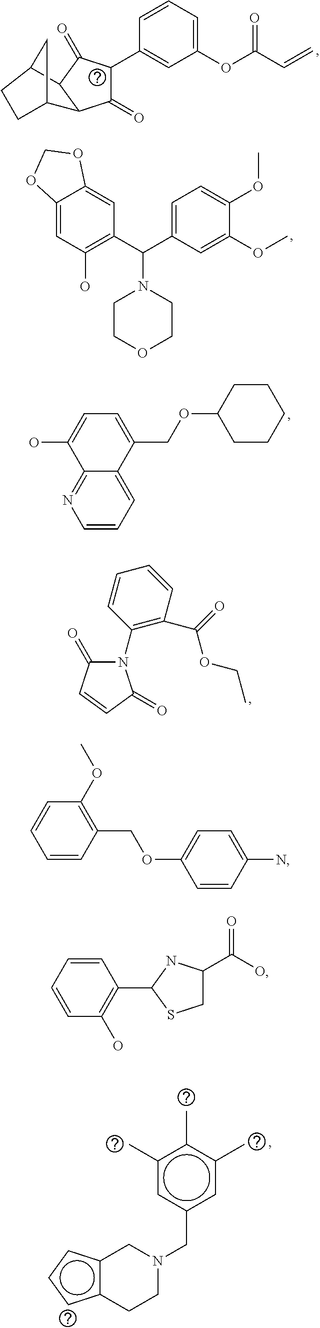

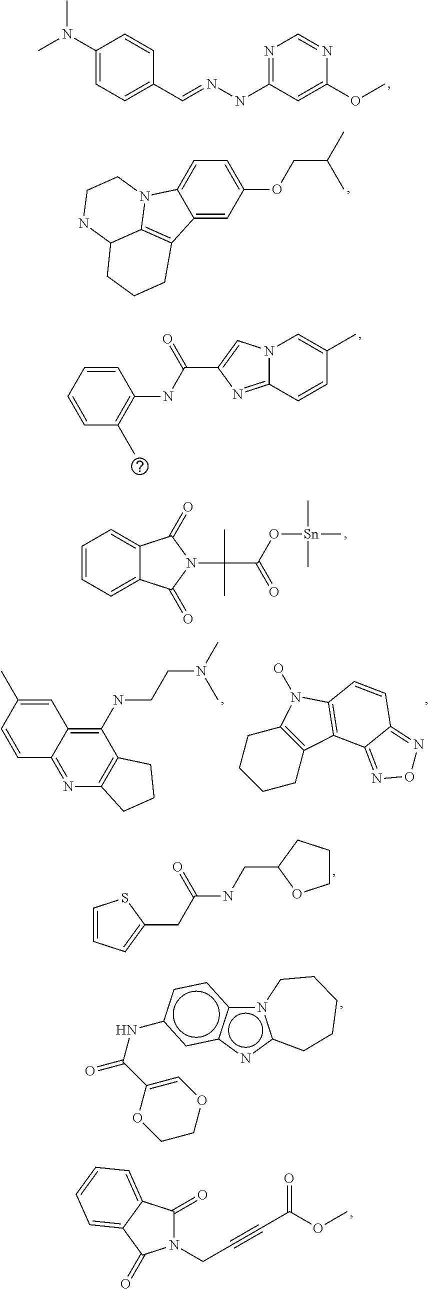

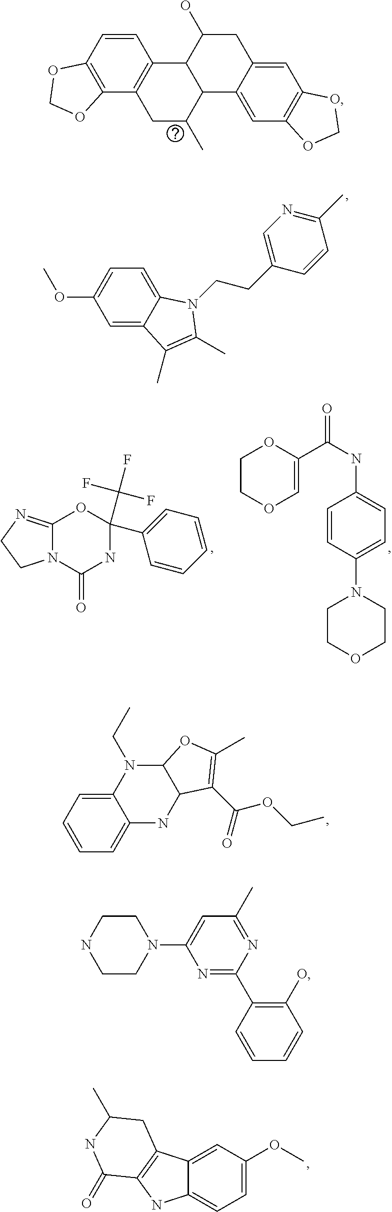

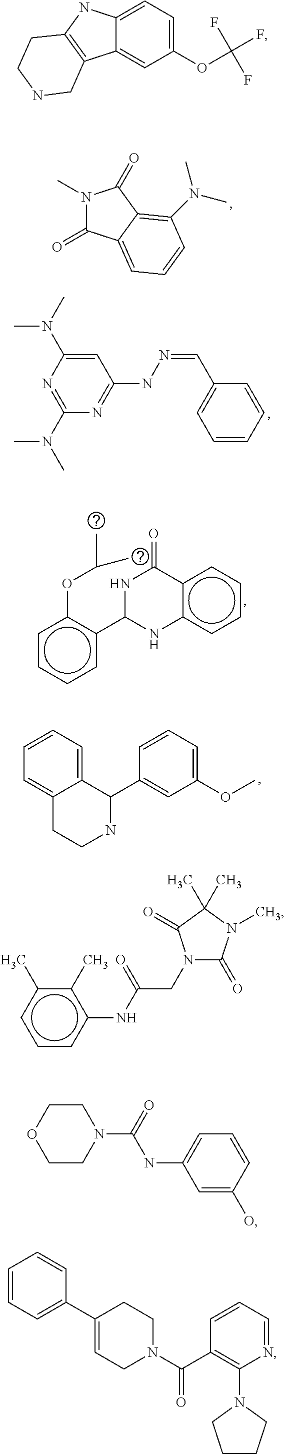

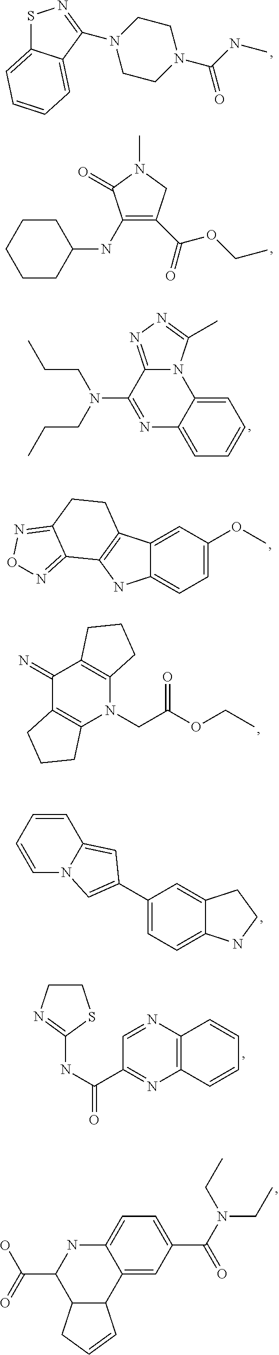

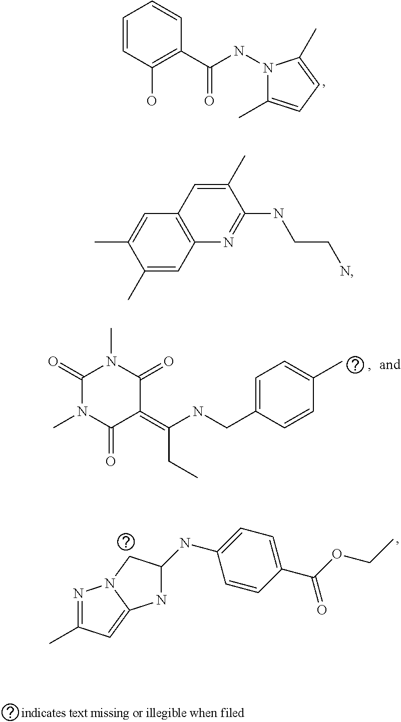

and pharmaceutically acceptable salts thereof.

In some embodiments, the composition comprises a compound selected from the group consisting of

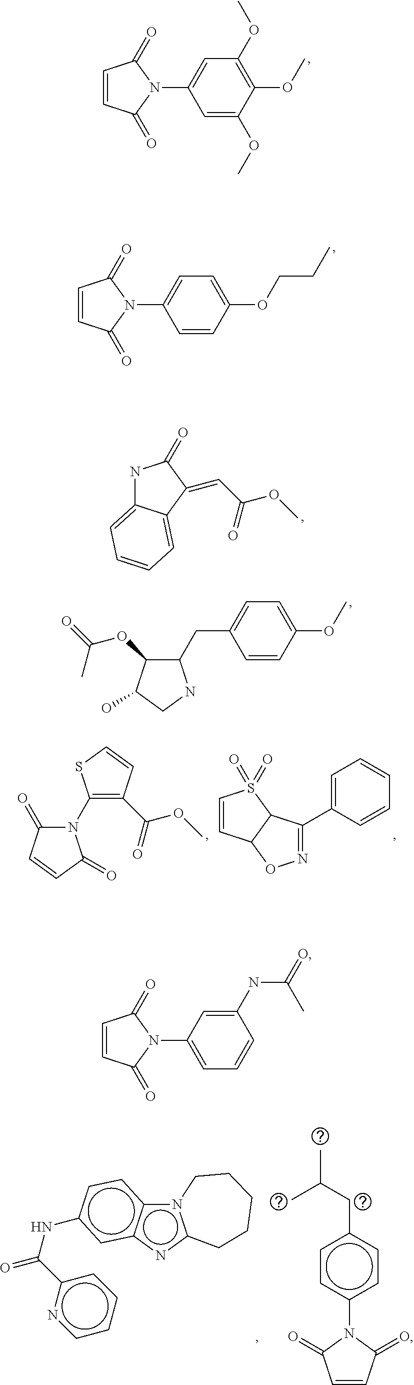

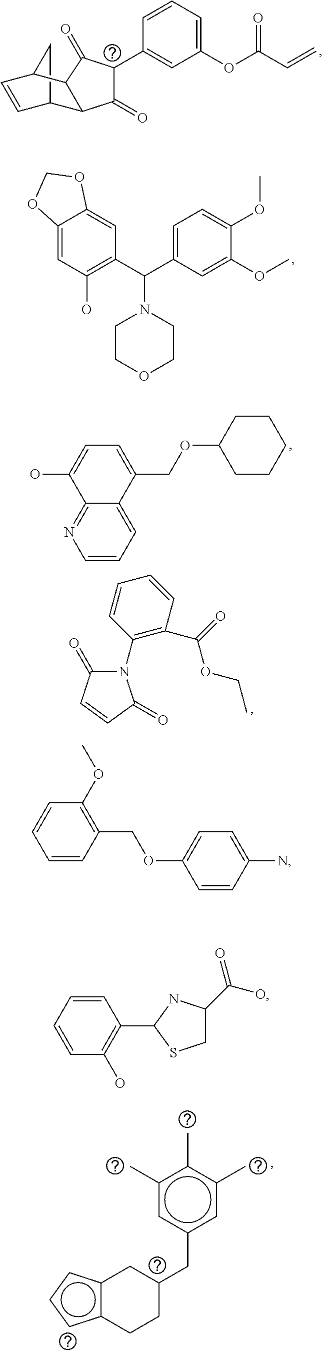

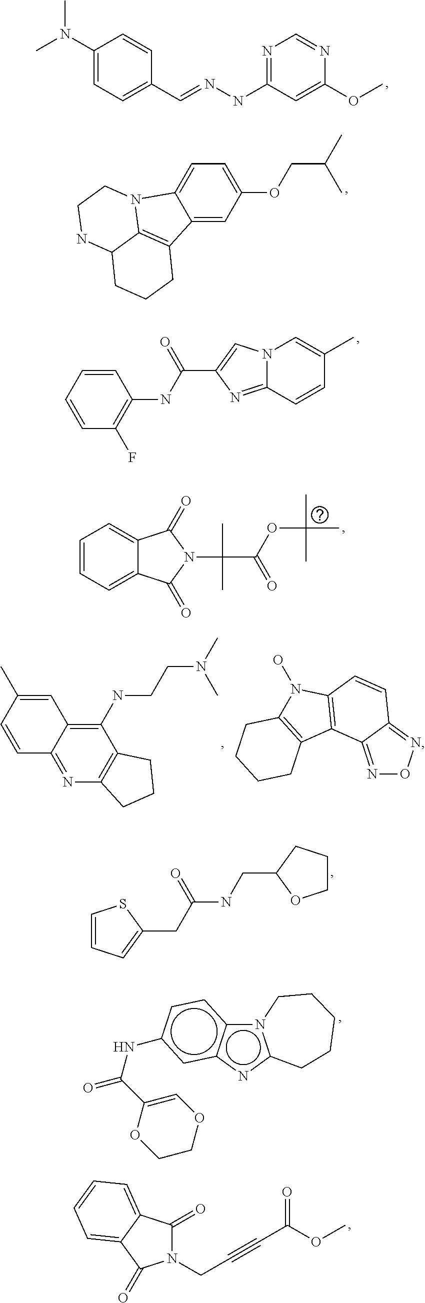

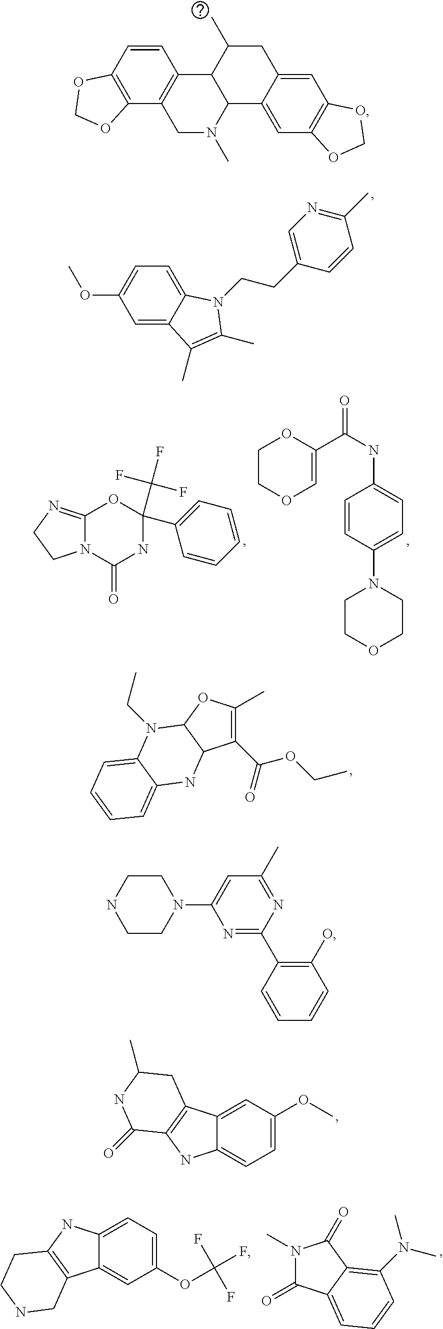

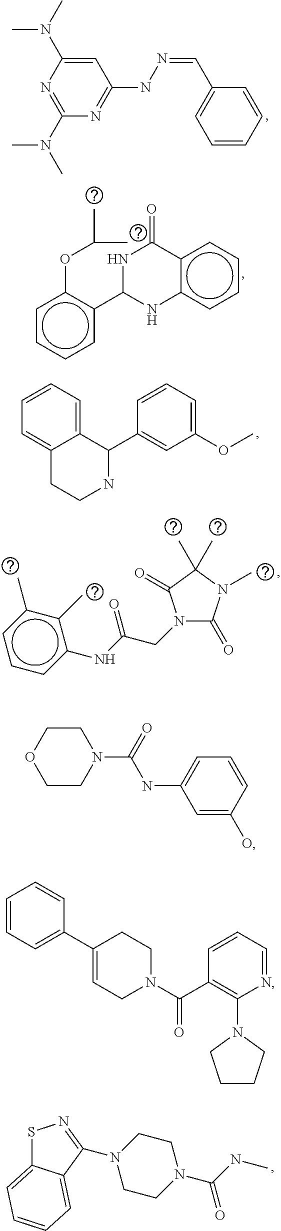

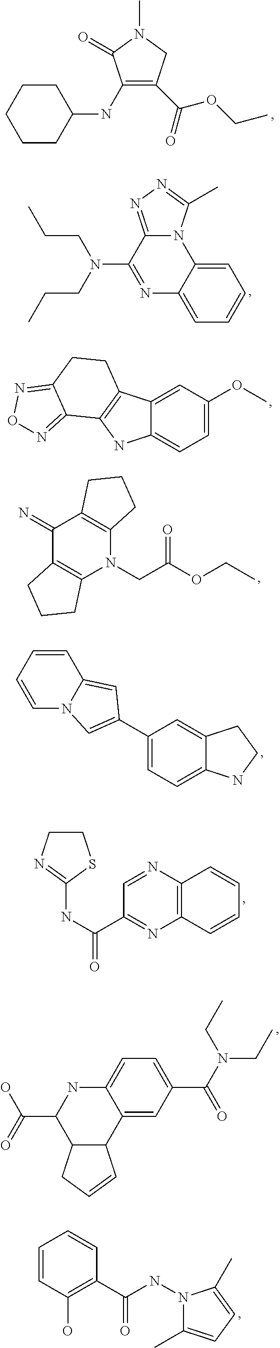

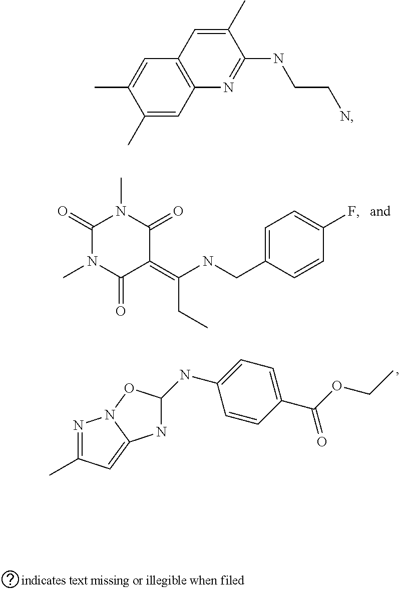

and pharmaceutically acceptable salts thereof.

In some embodiments, the composition comprises

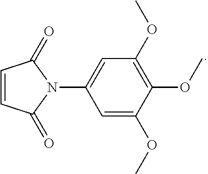 [SMILES: COc1cc(N2C(=O)C=CC2=O)cc(OC)c1OC]

In some embodiments, the composition comprises

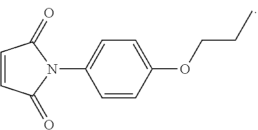 [SMILES: CCCOc1ccc(N2C(=O)C=CC2=O)cc1]

In some embodiments, the composition comprises

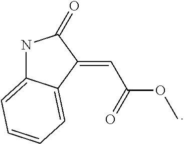

In some embodiments, the composition comprises

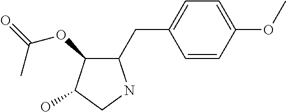 [SMILES: COc1ccc(CC2NC[C@H](O)[C@H]2OC(C)=O)cc1]

In some embodiments, the composition comprises

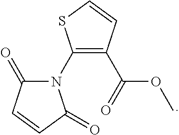 [SMILES: COC(=O)c1ccsc1N1C(=O)C=CC1=O]

In some embodiments, the composition comprises

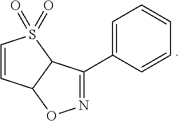 [SMILES: O=S1(=O)C=CC2ON=C(c3ccccc3)C21]

In some embodiments, the composition comprises

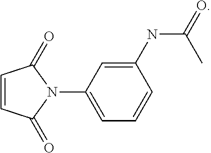 [SMILES: CC(=O)Nc1cccc(N2C(=O)C=CC2=O)c1]

In some embodiments, the composition comprises

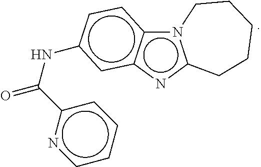

In some embodiments, the composition comprises

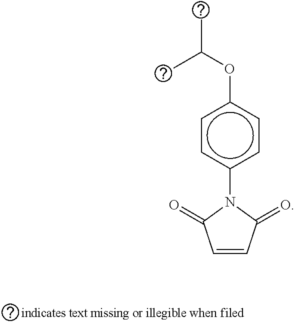

In some embodiments, the composition comprises

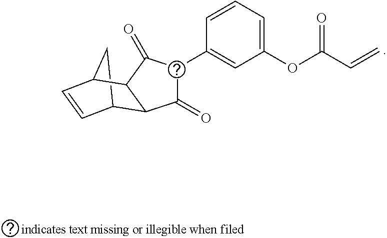 [SMILES: C=CC(=O)Oc1cccc(C2C(=O)C3C4C=CC(C4)C3C2=O)c1]

In some embodiments, the composition comprises

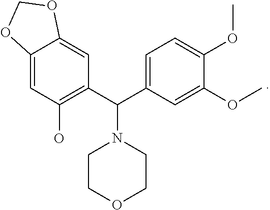 [SMILES: COc1ccc(C(c2cc3c(cc2O)OCO3)N2CCOCC2)cc1OC]

In some embodiments, the composition comprises

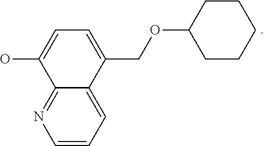 [SMILES: Oc1ccc(COC2CCCCC2)c2cccnc12]

In some embodiments, the composition comprises

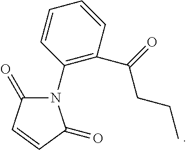 [SMILES: CCCC(=O)c1ccccc1N1C(=O)C=CC1=O]

In some embodiments, the composition comprises

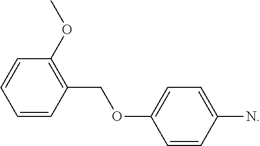

In some embodiments, the composition comprises

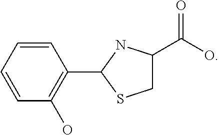

In some embodiments, the composition comprises

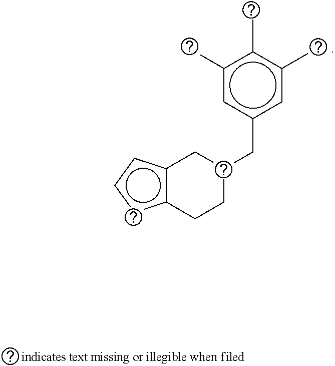

In some embodiments, the composition comprises

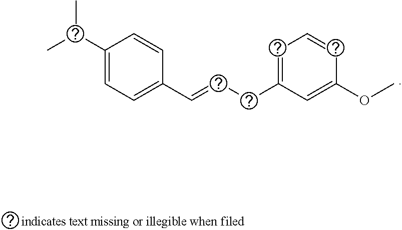 [SMILES: COc1cccc(C/C=C/c2ccc(C(C)C)cc2)c1]

In some embodiments, the composition comprises

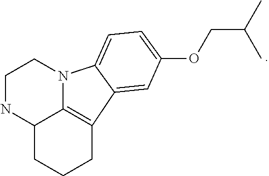 [SMILES: CC(C)COc1ccc2c(c1)c1c3n2CCNC3CCC1]

In some embodiments, the composition comprises

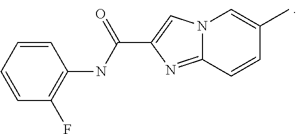 [SMILES: Cc1ccc2nc(C(=O)Nc3ccccc3F)cn2c1]

In some embodiments, the composition comprises

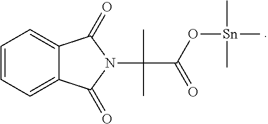 [SMILES: CC(C)(C(=O)[O][Sn]([CH3])([CH3])[CH3])N1C(=O)c2ccccc2C1=O]

In some embodiments, the composition comprises

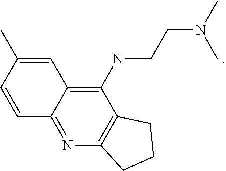 [SMILES: Cc1ccc2nc3c(c(NCCN(C)C)c2c1)CCC3]

In some embodiments, the composition comprises

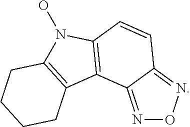 [SMILES: On1c2c(c3c4nonc4ccc31)CCCC2]

In some embodiments, the composition comprises

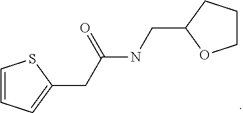 [SMILES: O=C(Cc1cccs1)NCC1CCCO1]

In some embodiments, the composition comprises

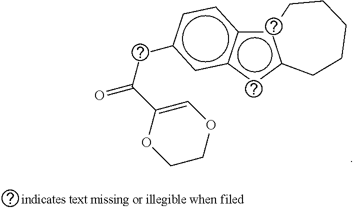

In some embodiments, the composition comprises

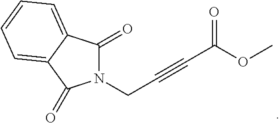 [SMILES: COC(=O)C#CCN1C(=O)c2ccccc2C1=O]

In some embodiments, the composition comprises

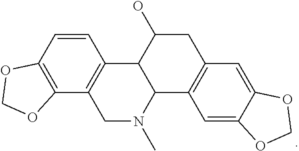 [SMILES: CN1Cc2c(ccc3c2OCO3)C2C(O)Cc3cc4c(cc3C21)OCO4]

In some embodiments, the composition comprises

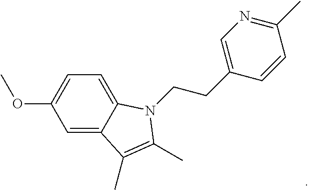

In some embodiments, the composition comprises

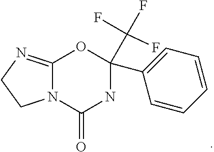 [SMILES: O=C1NC(c2ccccc2)(C(F)(F)F)OC2=NCCN12]

In some embodiments, the composition comprises

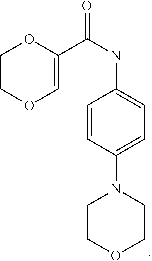 [SMILES: O=C(Nc1ccc(N2CCOCC2)cc1)C1=COCCO1]

In some embodiments, the composition comprises

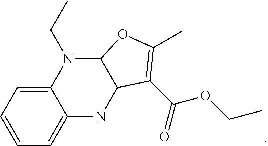 [SMILES: CCOC(=O)C1=C(C)OC2C1Nc1ccccc1N2CC]

In some embodiments, the composition comprises

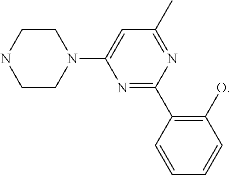

In some embodiments, the composition comprises

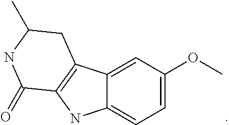 [SMILES: COc1ccc2[nH]c3c(c2c1)CC(C)NC3=O]

In some embodiments, the composition comprises

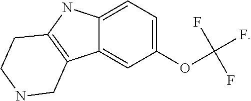 [SMILES: FC(F)(F)Oc1ccc2[nH]c3c(c2c1)CNCC3]

In some embodiments, the composition comprises

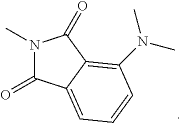

In some embodiments, the composition comprises

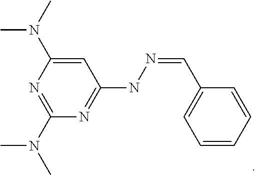 [SMILES: CN(C)c1cc(N/N=C\c2ccccc2)nc(N(C)C)n1]

In some embodiments, the composition comprises

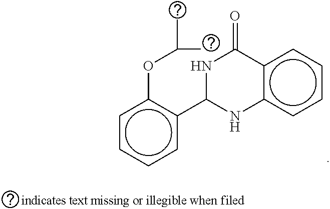

In some embodiments, the composition comprises

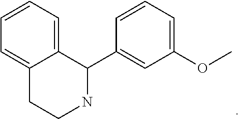 [SMILES: COc1cccc(C2NCCc3ccccc32)c1]

In some embodiments, the composition comprises

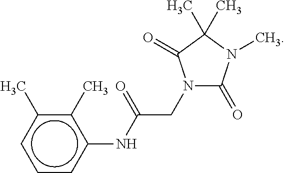

In some embodiments, the composition comprises

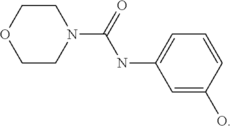

In some embodiments, the composition comprises

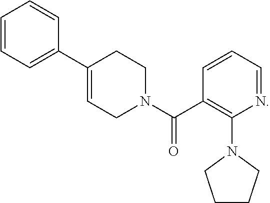 [SMILES: O=C(c1cccnc1N1CCCC1)N1CC=C(c2ccccc2)CC1]

In some embodiments, the composition comprises

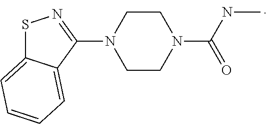 [SMILES: CNC(=O)N1CCN(c2nsc3ccccc23)CC1]

In some embodiments, the composition comprises

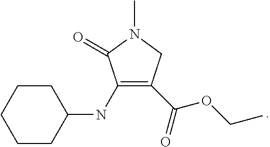 [SMILES: CCOC(=O)C1=C(NC2CCCCC2)C(=O)N(C)C1]

In some embodiments, the composition comprises

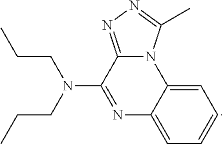 [SMILES: CCCN(CCC)c1nc2ccccc2n2c(C)nnc12]

In some embodiments, the composition comprises

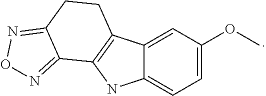 [SMILES: COc1ccc2[nH]c3c(c2c1)CCc1nonc1-3]

In some embodiments, the composition comprises

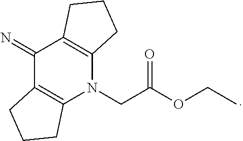 [SMILES: CCOC(=O)Cn1c2c(c(=N)c3c1CCC3)CCC2]

In some embodiments, the composition comprises

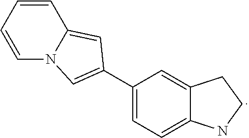 [SMILES: c1ccn2cc(-c3ccc4c(c3)CCN4)cc2c1]

In some embodiments, the composition comprises

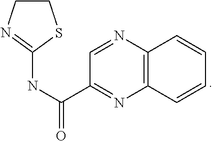 [SMILES: O=C(NC1=NCCS1)c1cnc2ccccc2n1]

In some embodiments, the composition comprises

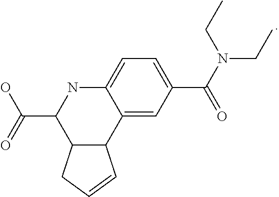 [SMILES: CCN(CC)C(=O)c1ccc2c(c1)C1C=CCC1C(C(=O)O)N2]

In some embodiments, the composition comprises

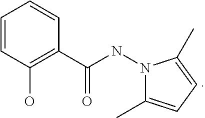 [SMILES: Cc1ccc(C)n1NC(=O)c1ccccc1O]

In some embodiments, the composition comprises

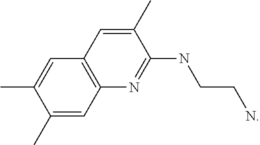

In some embodiments, the composition comprises

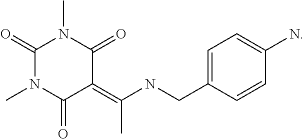

In some embodiments, the composition comprises

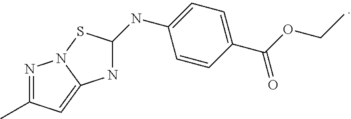 [SMILES: CCOC(=O)c1ccc(NC2Nc3cc(C)nn3S2)cc1]

In some embodiments, the composition comprises etoposide, 5-fluorouracil, or Clostridium difficile Toxin B. In some embodiments, the composition comprises etoposide. In some embodiments, the composition comprises 5-fluorouracil. In some embodiments, the composition comprises Clostridium difficile Toxin B.

Pharmaceutical Compositions and Administration Therapy

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

An MGES protein (such as, e.g., Stat3, C/EBPβ, C/EBPδ, RunX1, FosL2, bHLH-B2, and ZNF238) or an MGES modulating compound can be administered to the subject one time (e.g., as a single injection or deposition). Alternatively, and MGES protein or compounds of the invention can be administered once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12 times per year, or a combination thereof. Furthermore, an MGES protein or a MGES modulating compound can be co-administrated with another therapeutic, such as a chemotherapy drug.

Some non-limiting examples of conventional chemotherapy drugs include: aminoglutethimide, amsacrine, asparaginase, bcg, anastrozole, bleomycin, buserelin, bicalutamide, busulfan, capecitabine, carboplatin, camptothecin, chlorambucil, cisplatin, carmustine, cladribine, colchicine, cyclophosphamide, cytarabine, dacarbazine, cyproterone, clodronate, daunorubicin, diethylstilbestrol, docetaxel, dactinomycin, doxorubicin, dienestrol, etoposide, exemestane, filgrastim, fluorouracil, fludarabine, fludrocortisone, epirubicin, estradiol, gemcitabine, genistein, estramustine, fluoxymesterone, flutamide, goserelin, leuprolide, hydroxyurea, idarubicin, levamisole, imatinib, lomustine, ifosfamide, megestrol, melphalan, interferon, irinotecan, letrozole, leucovorin, ironotecan, mitoxantrone, nilutamide, medroxyprogesterone, mechlorethamine, mercaptopurine, mitotane, nocodazole, octreotide, methotrexate, mitomycin, paclitaxel, oxaliplatin, temozolomide, pentostatin, plicamycin, suramin, tamoxifen, porfimer, mesna, pamidronate, streptozocin, teniposide, procarbazine, titanocene dichloride, raltitrexed, rituximab, testosterone, thioguanine, vincristine, vindesine, thiotepa, topotecan, tretinoin, vinblastine, trastuzumab, and vinorelbine.

In some embodiments, the chemotherapy drug is an alkylating agent, a nitrosourea, an anti-metabolite, a topoisomerase inhibitor, a mitotic inhibitor, an anthracycline, a corticosteroid hormone, a sex hormone, or a targeted anti-tumor compound.

A targeted anti-tumor compound is a drug designed to attack cancer cells more specifically than standard chemotherapy drugs can. Most of these compounds attack cells that harbor mutations of certain genes, or cells that overexpress copies of these genes. In some embodiments, the anti-tumor compound can be imatinib (Gleevec), gefitinib (Iressa), erlotinib (Tarceva), rituximab (Rituxan), or bevacizumab (Avastin).

An alkylating agent works directly on DNA to prevent the cancer cell from propagating. These agents are not specific to any particular phase of the cell cycle. In some embodiments, alkylating agents can be selected from busulfan, cisplatin, carboplatin, chlorambucil, cyclophosphamide, ifosfamide, dacarbazine (DTIC), mechlorethamine (nitrogen mustard), melphalan, and temozolomide.

An antimetabolite makes up the class of drugs that interfere with DNA and RNA synthesis. These agents work during the S phase of the cell cycle and are commonly used to treat leukemias, tumors of the breast, ovary, and the gastrointestinal tract, as well as other cancers. In some embodiments, an antimetabolite can be 5-fluorouracil, capecitabine, 6-mercaptopurine, methotrexate, gemcitabine, cytarabine (ara-C), fludarabine, or pemetrexed.

Topoisomerase inhibitors are drugs that interfere with the topoisomerase enzymes that are important in DNA replication. Some examples of topoisomerase I inhibitors include topotecan and irinotecan while some representative examples of topoisomerase II inhibitors include etoposide (VP-16) and teniposide.

Anthracyclines are chemotherapy drugs that also interfere with enzymes involved in DNA replication. These agents work in all phases of the cell cycle and thus, are widely used as a treatment for a variety of cancers. In some embodiments, an anthracycline used with respect to the invention can be daunorubicin, doxorubicin (Adriamycin), epirubicin, idarubicin, or mitoxantrone.

An MGES protein or an MGES modulating compound of the invention can be administered to a subject by any means suitable for delivering the protein or compound to cells of the subject. For example, it can be administered by methods suitable to transfect cells. Transfection methods for eukaryotic cells are well known in the art, and include direct injection of the nucleic acid into the nucleus or pronucleus of a cell; electroporation; liposome transfer or transfer mediated by lipophilic materials; receptor mediated nucleic acid delivery, bioballistic or particle acceleration; calcium phosphate precipitation, and transfection mediated by viral vectors.

The compositions of this invention can be formulated and administered to reduce the symptoms associated with a nervous system cancer (e.g, a glioma) by any means that produce contact of the active ingredient with the agent's site of action in the body of a human or non-human subject. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's Pharmaceutical Sciences, Meade Publishing Co., Easton, Pa. (20^{1h} ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers, such as PBS, Hank's solution, or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

Any of the therapeutic applications described herein can be applied to any subject in need of such therapy, including, for example, a mammal such as a dog, a cat, a cow, a horse, a rabbit, a monkey, a pig, a sheep, a goat, or a human. Thus, in some embodiments, the subject is a mammal. In some embodiments, the subject is a dog, a cat, a cow, a horse, a rabbit, a monkey, a pig, a sheep, a goat, or a human. In some embodiments, the subject is a dog, a monkey, or a human. In some embodiments, the subject is a human.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition must be sterile and fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the MGES modulating compound in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art

A composition of the invention can be administered to a subject in need thereof. Subjects in need thereof can include but are not limited to, for example, a mammal such as a dog, a cat, a cow, a horse, a rabbit, a monkey, a pig, a sheep, a goat, or a human. Thus, in some embodiments, the subject is a mammal. In some embodiments, the subject is a dog, a cat, a cow, a horse, a rabbit, a monkey, a pig, a sheep, a goat, or a human. In some embodiments, the subject is a dog, a monkey, or a human. In some embodiments, the subject is a human.

A composition of the invention can also be formulated as a sustained and/or timed release formulation. Such sustained and/or timed release formulations can be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556; and 5,733,566, the entire disclosures of which are each incorporated herein by reference. The pharmaceutical compositions of the invention (e.g, that have a therapeutic effect) can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, can be readily selected for use with the pharmaceutical compositions of the invention. Single unit dosage forms suitable for oral administration, such as, but not limited to, tablets, capsules, gel-caps, caplets, or powders, that are adapted for sustained release are encompassed by the invention.

In the methods described herein, an MGES protein or a MGES modulating compound can be administered to the subject either as RNA, in conjunction with a delivery reagent, or as a nucleic acid (e.g., a recombinant plasmid or viral vector) comprising sequences which express the gene product. Suitable delivery reagents for administration of the MGES protein or compounds include the Mirus Transit TKO lipophilic reagent; lipofectin; lipofectamine; cellfectin; or polycations (e.g., polylysine), or liposomes.

The dosage administered can be a therapeutically effective amount of the composition sufficient to result in amelioration of symptoms of a nervous system cancer in a subject (e.g, a decrease or inhibition of nervous system tumor cell proliferation, a decrease or inhibition of angiogenesis), and can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

In some embodiments, the effective amount of the administered MGES polypetide, MGES, polynucleotide, or MGES modulating compound is at least about 0.01 μg/kg body weight, at least about 0.025 μg/kg body weight, at least about 0.05 μg/kg body weight, at least about 0.075 μg/kg body weight, at least about 0.1 μg/kg body weight, at least about 0.25 μg/kg body weight, at least about 0.5 μg/kg body weight, at least about 0.75 μg/kg body weight, at least about 1 μg/kg body weight, at least about 5 μg/kg body weight, at least about 10 μg/kg body weight, at least about 25 μg/kg body weight, at least about 50 μg/kg body weight, at least about 75 μg/kg body weight, at least about 100 μg/kg body weight, at least about 150 μg/kg body weight, at least about 200 μg/kg body weight, at least about 250 μg/kg body weight, at least about 300 μg/kg body weight, at least about 350 μg/kg body weight, at least about 400 μg/kg body weight, at least about 450 μg/kg body weight, at least about 500 μg/kg body weight, at least about 550 μg/kg body weight, at least about 600 μg/kg body weight, at least about 650 μg/kg body weight, at least about 700 μg/kg body weight, at least about 750 μg/kg body weight, at least about 800 μg/kg body weight, at least about 850 μg/kg body weight, at least about 900 μg/kg body weight, at least about 950 μg/kg body weight, or at least about 1000 μg/kg body weight.

In some embodiments, the effective amount of the administered MGES polypetide, MGES, polynucleotide, or MGES modulating compound is at least about 0.1 mg/kg body weight, at least about 0.3 mg/kg body weight, at least about 0.5 mg/kg body weight, at least about 0.75 mg/kg body weight, at least about 1 mg/kg body weight, at least about 5 mg/kg body weight, at least about 10 mg/kg body weight, at least about 25 mg/kg body weight, at least about 50 mg/kg body weight, at least about 75 mg/kg body weight, at least about 100 mg/kg body weight, at least about 150 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 850 mg/kg body weight, at least about 900 mg/kg body weight, at least about 950 mg/kg body weight, or at least about 1000 mg/kg body weight.

In some embodiments, an MGES protein or a MGES modulating compound is administered at least once daily. In some embodiments, an MGES protein or a MGES modulating compound is administered at least twice daily. In some embodiments, an MGES protein or a MGES modulating compound is administered for at least 1 week, for at least 2 weeks, for at least 3 weeks, for at least 4 weeks, for at least 5 weeks, for at least 6 weeks, for at least 8 weeks, for at least 10 weeks, for at least 12 weeks, for at least 18 weeks, for at least 24 weeks, for at least 36 weeks, for at least 48 weeks, or for at least 60 weeks. In some embodiments, an MGES protein and/or an MGES modulating compound is administered in combination with a second therapeutic agent.

Toxicity and therapeutic efficacy of therapeutic compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutic agents that exhibit large therapeutic indices are useful. Therapeutic compositions that exhibit some toxic side effects can be used.

Gene Therapy and Protein Replacement Methods

In one aspect, the invention provides methods for treating a nervous system cancer in a subject, e.g., a glioma. In some embodiments, the method can comprise administering to the subject an MGES molecule (e.g, a MGES polypeptide or a MGES polynucleotide) or a MGES modulating compound, which can be a polypeptide, small molecule, antibody, or a nucleic acid.

Various approaches can be carried out to restore the activity or function of an MGES gene (such as, e.g., Stat3, C/EBPβ, C/EBPδ, RunX1, FosL2, bHLH-B2, or ZNF238) in a subject, such as those carrying an altered MGES gene locus. For example, supplying wild-type MGES gene function (such as, e.g., Stat3, C/EBPβ, C/EBPδ, RunX1, FosL2, bHLH-B2, or ZNF238) to such subjects can suppress the phenotype of a nervous system cancer in a subject (e.g., nervous system tumor cell proliferation, mervous system tumor size, or angiogenesis). Increasing and/or decreasing MGES gene expression levels or activity (such as, e.g., Stat3, C/EBPβ, C/EBPδ, RunX1, FosL2, bHLH-B2, or ZNF238) can be accomplished through gene or protein therapy.

A nucleic acid encoding an MGES gene, or a functional part thereof can be introduced into the cells of a subject. For example, the wild-type gene (or a functional part thereof) can also be introduced into the cells of the subject in need thereof using a vector as described herein. The vector can be a viral vector or a plasmid. The gene can also be introduced as naked DNA. The gene can be provided so as to integrate into the genome of the recipient host cells, or to remain extra-chromosomal. Integration can occur randomly or at precisely defined sites, such as through homologous recombination. For example, a functional copy of an MGES gene can be inserted in replacement of an altered version in a cell, through homologous recombination. Further techniques include gene gun, liposome-mediated transfection, or cationic lipid-mediated transfection. Gene therapy can be accomplished by direct gene injection, or by administering ex vivo prepared genetically modified cells expressing a functional polypeptide.

Delivery of nucleic acids into viable cells can be effected ex vivo, in situ, or in vivo by use of vectors, and more particularly viral vectors (e.g., lentivirus, adenovirus, adeno-associated virus, or a retrovirus), or ex vivo by use of physical DNA transfer methods (e.g., liposomes or chemical treatments). Non-limiting techniques suitable for the transfer of nucleic acid into mammalian cells in vitro include the use of liposomes, electroporation, microinjection, cell fusion, DEAE-dextran, and the calcium phosphate precipitation method (see, for example, Anderson, Nature, supplement to vol. 392, no. 6679, pp. 25-20 (1998); herein incorporated by reference in its entirety). Introduction of a nucleic acid or a gene encoding a polypeptide of the invention can also be accomplished with extrachromosomal substrates (transient expression) or artificial chromosomes (stable expression). Cells may also be cultured ex vivo in the presence of therapeutic compositions of the present invention in order to proliferate or to produce a desired effect on or activity in such cells. Treated cells can then be introduced in vivo for therapeutic purposes.

Nucleic acids can be inserted into vectors and used as gene therapy vectors. A number of viruses have been used as gene transfer vectors, including papovaviruses, e.g., SV40 (Madzak et al., 1992; herein incorporated by reference in its entirety), adenovirus (Berkner, 1992; Berkner et al., 1988; Gorziglia and Kapikian, 1992; Quantin et al., 1992; Rosenfeld et al., 1992; Wilkinson et al., 1992; Stratford-Perricaudet et al., 1990; each herein incorporated by reference in its entirety), vaccinia virus (Moss, 1992; herein incorporated by reference in its entirety), adeno-associated virus (Muzyczka, 1992; Ohi et al., 1990; each herein incorporated by reference in its entirety), herpesviruses including HSV and EBV (Margolskee, 1992; Johnson et al., 1992; Fink et al., 1992; Breakfield and Geller, 1987; Freese et al., 1990; each herein incorporated by reference in its entirety), and retroviruses of avian (Biandyopadhyay and Temin, 1984; Petropoulos et al., 1992; each herein incorporated by reference in its entirety), murine (Miller, 1992; Miller et al., 1985; Sorge et al., 1984; Mann and Baltimore, 1985; Miller et al., 1988; each herein incorporated by reference in its entirety), and human origin (Shimada et al., 1991; Helseth et al., 1990; Page et al., 1990; Buchschacher and Panganiban, 1992; each herein incorporated by reference in its entirety). Non-limiting examples of in vivo gene transfer techniques include transfection with viral (typically retroviral) vectors (see U.S. Pat. No. 5,252,479; herein incorporated by reference in its entirety) and viral coat protein-liposome mediated transfection (Dzau et al., Trends in Biotechnology 11:205-210 (1993); herein incorporated by reference in its entirety). For example, naked DNA vaccines are generally known in the art; see Brower, Nature Biotechnology, 16:1304-1305 (1998); herein incorporated by reference in its entirety. Gene therapy vectors can be delivered to a subject by, for example, intravenous injection, local administration (see, e.g., U.S. Pat. No. 5,328,470; herein incorporated by reference in its entirety) or by stereotactic injection (see, e.g., Chen, et al., 1994. Proc. Natl. Acad. Sci. USA 91: 3054-3057; herein incorporated by reference in its entirety). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

For reviews of gene therapy protocols and methods see Anderson et al., Science 256:808-813 (1992); U.S. Pat. Nos. 5,252,479, 5,747,469, 6,017,524, 6,143,290, 6,410,010 6,511,847; and U.S. Application Publication Nos. 2002/0077313 and 2002/00069; each herein incorporated by reference in its entirety. For additional reviews of gene therapy technology, see Friedmann, Science, 244:1275-1281 (1989); Verma, Scientific American: 68-84 (1990); Miller, Nature, 357: 455-460 (1992); Kikuchi et al., J Dermatol Sci. 2008 May; 50(2):87-98; Isaka et al., Expert Opin Drug Deliv. 2007 September; 4(5):561-71; Jager et al., Curr Gene Ther. 2007 August; 7(4):272-83; Waehler et al., Nat Rev Genet. 2007 August; 8(8):573-87; Jensen et al., Ann Med. 2007; 39(2):108-15; Herweijer et al., Gene Ther. 2007 January; 14(2):99-107; Eliyahu et al., Molecules, 2005 Jan. 31; 10(1):34-64; and Altaras et al., Adv Biochem Eng Biotechnol. 2005; 99:193-260; each herein incorporated by reference in its entirety.

Protein replacement therapy can increase the amount of protein by exogenously introducing wild-type or biologically functional protein by way of infusion. A replacement polypeptide can be synthesized according to known chemical techniques or may be produced and purified via known molecular biological techniques. Protein replacement therapy has been developed for various disorders. For example, a wild-type protein can be purified from a recombinant cellular expression system (e.g., mammalian cells or insect cells-see U.S. Pat. No. 5,580,757 to Desnick et al.; U.S. Pat. Nos. 6,395,884 and 6,458,574 to Selden et al.; U.S. Pat. No. 6,461,609 to Calhoun et al.; U.S. Pat. No. 6,210,666 to Miyamura et al.; U.S. Pat. No. 6,083,725 to Selden et al.; U.S. Pat. No. 6,451,600 to Rasmussen et al.; U.S. Pat. No. 5,236,838 to Rasmussen et al. and U.S. Pat. No. 5,879,680 to Ginns et al.; each herein incorporated by reference in its entirety), human placenta, or animal milk (see U.S. Pat. No. 6,188,045 to Reuser et al.; herein incorporated by reference in its entirety), or other sources known in the art. After the infusion, the exogenous protein can be taken up by tissues through non-specific or receptor-mediated mechanism.

These methods described herein are by no means all-inclusive, and further methods to suit the specific application is understood by the ordinary skilled artisan. Moreover, the effective amount of the compositions can be further approximated through analogy to compounds known to exert the desired effect.

Nervous System Tumors and Tumor Targets

In some embodiments, the invention can be used to treat various nervous system tumors, for example gliomas (e.g., astrocytomas (such as anaplastic astrocytoma), Glioblastoma Multiforme (GBM), oligodendrogliomas, ependymoma) and meningiomas. The nervous system tumor can include, but is not limited to, cerebellar astrocytoma, medulloblastoma, ependymona, brain stem glioma, optic nerve glioma, acoustic neuromas, nerve sheath tumors, or germinoma. In some embodiments, the methods for treating cancer relate to methods for inhibiting proliferation of a cancer or tumor cell comprising administering to a subject a protein or other agent that decreases expression of a MGES gene (e.g., Stat3, C/EBPβ, C/EBPδ, RunX1, FosL2, bHLH-B2, ZNF238, or a combination thereof) of the tumor or cancer cell.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

Those skilled in the art will recognize, or be able to ascertain using no more than routine experimentation, many equivalents to the specific embodiments of the invention described herein. Such equivalents are intended to be within the scope of the present invention.

The invention is further described by the following non-limiting Examples.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Id Proteins Stimulate Axonal Elongation

Recent work from the laboratory identified a new and unexpected function for Id proteins, namely the ability to stimulate axonal elongation (Iavarone and Lasorella, 2006; Lasorella et al., 2006; each herein incorporated by reference in its entirety). These studies originated from the identification of the Anaphase Promoting Complex (APC) as the ubiquitin ligase that primes Id2 for proteasomal-mediated degradation. Degradation of Id2 by APC is mediated by a highly conserved sequence, the destruction box (D-box), which is required for recognition by the APC co-activator Cdhl. It was found that mutation of the D-box of Id2 (Id2-DBM) resulted in marked stabilization of the protein in neural cells. Previous work had shown that APC-Cdhl restrains axonal growth in different types of CNS neurons (Konishi et al., 2004; herein incorporated by reference in its entirety). However, the natural targets of APC-Cdhl for the axonal growth phenotype remained unknown. The recent studies identified those targets. It was found that introduction of Id2-DBM in cortical and cerebellar neurons was sufficient to enhance axonal growth and override the inhibitory effects on axonal elongation imposed by myelin components. These effects are implemented by Id2-mediated silencing of a gene expression response induced by bHLH transcription factors. The products of the bHLHinducible genes repressed by Id2 in neurons are secreted molecules (Sema3F), ligands (jagged-2) and receptors (Nogo Recepotor, Unc5A, Notch-I) of multiple inhibitory and repellant signals for axons (Barallobre et al., 2005; Fiore and Puschel, 2003; Lesuisse and Martin, 2002; Sestan et al., 1999; Spencer et al., 2003; each herein incorporated by reference in its entirety).

Recovery following spinal cord injury (SCI) is limited because severed axons of the CNS fail to regenerate. Neverthless, some recovery of sensory and motor functions occurs over the first few weeks following incomplete injuries. Without being bound by theory, the most important Mechanism responsible for this recovery is the trigger of injury-induced plasticity, a phenomenon manifested by the establishment of new intraspinal circuits in the lesioned area. Although the mechanisms promoting injury-induced plasticity are poorly understood, an important event is up-regulation of genes that stimulate axonal growth and neurotrophic factors (jun, NT-3, BDNF, etc.) (Becker and Bonni, 2005; herein incorporated by reference in its entirety). Remarkably, injury of many types of neurons in vivo is associated with upregulation of Id genes. Without being bound by theory, expression of Id2 can generate beneficial effects for regeneration of damaged axons in the CNS.

Experimental Plan and Methods:

Here, the results observed in vitro following introduction of undegradable Id2 into neurons are extended to a mouse model of spinal cord injury. To do this, a pilot study will be performed using adeno-associated viruses (AAV) encoding Id2-DBM in mice that have received a spinal cord injury. Without being bound by theory, mice transduced with AAV-Id2-DBM will regenerate axons more efficiently than control mice (infected with AAVGFP) and display greater functional locomotor recovery.

Delivery of Virus.

The delivery system to be used is injection of the sensory-motor cortex with the AAV-based constructs. AAV is the most effective system to introduce exogenous proteins in post-mitotic neurons in the adult animal (Kaspar et al., 2003; Xiao et al., 1997; each herein incorporated by reference in its entirety). The most striking aspect of AAV transduction in the CNS is the absence of expression of the exogenous gene in glial cells (Burger et al., 2004; Passini et al., 2006; each herein incorporated by reference in its entirety). The AAV5 serotype was selected based on its superior ability to transducer mammalian brain in comparison with the other AAV serotypes (Passini et al., 2006; herein incorporated by reference in its entirety).

AAV5-Id2-DBM and AAV5-GFP will be produced and purified by Virapur (San Diego, Calif.) by cotransfection of Helper plasmid and a plasmid expressing the AAV5 rep and cap genes. To evaluate whether introduction of Id2-DBM promotes axonal regeneration in the CST, 5 μl of each viral preparation (approximate titer: 2×10¹¹ genome copies/ml) will be stereotactically injected into the motor cortex of 20 mice (10 with AAV-GFP, 10 with AAV-Id2-DBM) using a single needle tract. In an additional group of 20 mice the AAVs will be injected directly in the spinal cord to transduce propriospinal neurons and evaluate whether Id2-DBM stimulates formation of new circuits and leads to better functional recovery in the behavioral tests. The total of 40 mice will undergo lateral hemisection injury of the thoracic spinal cord with severing of the dorsal cortico-spinal tract (CST) in the dorsal funiculus as well as the lateral CST. During the same operation as the lesion procedure, animals will be randomly divided into the two experimental groups (20 mice injected with AAVGFP, 20 mice injected with AAV-Id2-DBM) and will undergo stereotactic injection with each virus in the sensory-motor cortex controlateral to the lesion site or will be directly injected in the lesioned area of the spinal cord. The study will be terminated three months after SCI/AAV injection when the animals will be analyzed with end-point behavioral tests and sacrificed for pathological examination. Surgical and behavioral procedures will be performed at the CRF SCI Core, after which perfused, collected tissue will be shipped for histological analysis.

Behavioral Testing.

Animals will be monitored to analyze behavioral recovery weekly for nine weeks after injury in an open field environment by the BBB. Quantification will be performed in a blinded manner by two observers. Three months after lesion and just before sacrificing, the animals will be videotaped on a horizontal ladder beam test in a series of three trials and scored over 150 rungs by two independent observers. They will also undergo a final stage kinematic locomotor testing using CatWalk and DigiGait analysis. Results will be analyzed for statistically significant differences between the two experimental groups either a two-way ANOVA or by using a paired t test (significance <0.05).

Pathological Examination.

The integrity of the dorsal CST will be assessed by tracer (biotindextran amine, BDA) injection into the bilateral sensory-motor cortices 14 to 21 days prior to sacrifice. The retrograde tracer Fluorogold will also be injected below the injury site. Blocks extending 5 mm rostral and 5 mm caudal to the center of the injury will be sectioned in the sagittal plane. The far-rostral as well as the far-caudal segments will be sectioned in the transverse plane. The spinal cord will be dissected, fixed, embedded and sectioned. On each section the number of intersections of BDA-labeled fibers with a dorso-ventral line will be counted from 4 mm above to 4 mm below the lesion site. Axon number will be calculated as a percentage of the fibers seen 4 cm above the lesion where the CST is intact. For immunohistochemistry, frozen tissue will be obtained from an uninjured spinal cord and from each animal group.

### REFERENCES

Barallobre, M. J., Pascual, M., Del Rio, J. A., and Soriano, E. (2005). The Netrin family of guidance factors: emphasis on Netrin-1 signalling. Brain Res Brain Res Rev 49, 22-47.
Becker, E. B., and Bonni, A. (2005). Beyond proliferation—cell cycle control of neuronal survival and differentiation in the developing mammalian brain. Semin Cell Dev Biol 16, 439-448.
Burger, C., Gorbatyuk, O, S., Velardo, M. J., Peden, C. S., Williams, P., Zolotukhin, S., Reier, P. J., Mandel, R. J., and Muzyczka, N. (2004). Recombinant AAV viral vectors pseudotyped with viral capsids from serotypes 1, 2, and 5 display differential efficiency and cell tropism after delivery to different regions of the central nervous system. Mol Ther 10, 302-317.
Fiore, R., and Puschel, A. W. (2003). The function of semaphorins during nervous system development. Front Biosci 8, s484-499.
Iavarone, A., and Lasorella, A. (2004). Id proteins in neural cancer. Cancer Lett 204, 189-196.
Iavarone, A., and Lasorella, A. (2006). ID proteins as targets in cancer and tools in neurobiology. Trends Mol Med 12, 588-594.
Kaspar, B. K., Llado, J., Sherkat, N., Rothstein, J. D., and Gage, F. H. (2003). Retrograde viral delivery of IGF-1 prolongs survival in a mouse ALS model. Science 301, 839-842.
Konishi, Y., Stegmuller, J., Matsuda, T., Bonni, S., and Bonni, A. (2004). Cdhl-APC controls axonal growth and patterning in the mammalian brain. Science 303, 1026-1030.
Lasorella, A., Stegmuller, J., Guardavaccaro, D., Liu, G., Carro, M. S., Rothschild, G., de la Torre-Ubieta, L., Pagano, M., Bonni, A., and Iavarone, A. (2006). Degradation of Id2 by the anaphase-promoting complex couples cell cycle exit and axonal growth. Nature 442, 471-474.
Lasorella, A., Uo, T., and Iavarone, A. (2001). Id proteins at the cross-road of development and cancer. Oncogene 20, 8326-8333.
Lesuisse, C., and Martin, L. J. (2002). Long-term culture of mouse cortical neurons as a model for neuronal development, aging, and death. J Neurobiol 51, 9-23.
Norton, J. D., Deed, R. W., Craggs, G., and Sablitzky, F. (1998). Id helix-loop-helix proteins in cell growth and differentiation. Trends Cell Biol 8, 58-65.
Passini, M. A., Dodge, J. C., Bu, J., Yang, W., Zhao, Q., Sondhi, D., Hackett, N. R., Kaminsky, S. M., Mao, Q., Shihabuddin, L. S., et al. (2006). Intracranial delivery of CLN2 reduces brain pathology in a mouse model of classical late infantile neuronal ceroid lipofuscinosis. J Neurosci 26, 1334-1342.
Perk, J., Iavarone, A., and Benezra, R. (2005). Id family of helix-loop-helix proteins in cancer. Nat Rev Cancer 5, 603-614.
Sestan, N., Artavanis-Tsakonas, S., and Rakic, P. (1999). Contact-dependent inhibition of cortical neurite growth mediated by notch signaling. Science 286, 741-746.
Spencer, T., Domeniconi, M., Cao, Z., and Filbin, M. T. (2003). New roles for old proteins in adult CNS axonal regeneration. Curr Opin Neurobiol 13, 133-139.
Xiao, X., Li, J., McCown, T. J., and Samulski, R. J. (1997). Gene transfer by adeno-associated virus vectors into the central nervous system. Exp Neurol 144, 113-124.
Ying, Q. L., Nichols, J., Chambers, I., and Smith, A. (2003). BMP induction of Id proteins suppresses differentiation and sustains embryonic stem cell self-renewal in collaboration with STAT3. Cell 115, 281-292.

### Example 2

### Transcriptional Regulation Module in High-Grade Glioma

Computational Identification of the MGES Transcriptional Regulation Module in High-Grade Glioma.

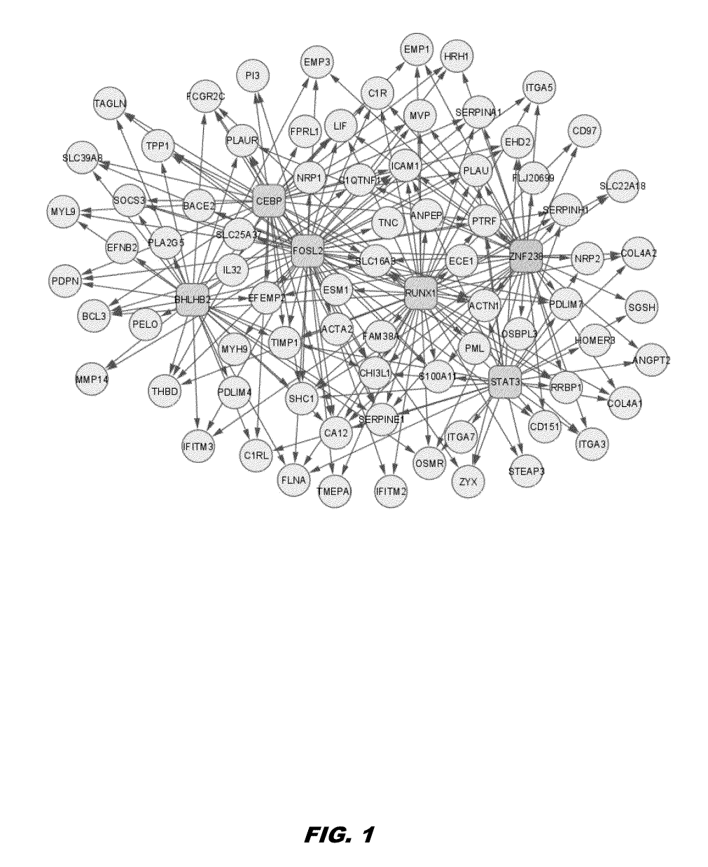

To identify Master Transcriptional Modules (MTM) and MRs of the MGES, ARACNe was applied to 176 AA and GBM samples (22, 66, 77; each herein incorporated by reference in its entirety), which had been previously classified into three molecular signature groups—proneural, proliferative, and mesenchymal (MGES) —by unsupervised cluster analysis (77; herein incorporated by reference in its entirety). The Master Regulator Analysis (MRA) algorithm was developed to infer a comprehensive repertoire of candidate MRs, regulating 102 genes that were overexpressed in the MGES. First, TFs were identified by their annotation in the Gene Ontology (3; herein incorporated by reference in its entirety). Then, for each TF the Fisher Exact Test (FET) was used to determine whether the intersection of its ARACNe predicted targets (the TF-regulon) with the MGES genes was statistically significant. From a global list of 1018 TFs, the MRA produced a subset of 55 MGES-specific, candidate MRs, at a False Discovery Rate, FDR ≦0.05. Among the 55 candidate MRs in the ARACNe network, the top six (Stat3, C/EBPβ/δ, bHLH-B2, Runx1, FosL2, and ZNF238) appear to collectively regulate 74% of the MGES genes (FIG. 1). This is a lower bound because ARACNe has a low false positive rate but a higher false negative rate. False negatives are not an issue in this analysis, as long as the number of TF-targets in the regulon is sufficient to assess statistically significant enrichment of MGES genes.

Multiple dataset and modality integration, using machine learning approaches such as Naïve Bayes classifiers, has been shown to significantly outperform individual analyses (36; herein incorporated by reference in its entirety). Additionally, since ARACNe trades off a low false-positive rate for a higher false-negative rate, appropriate integration of ARACNe's inferences from multiple datasets will be especially useful to achieve higher coverage of transcriptional interactions. Convergence of ARACNe inferences from distinct datasets was successfully shown (49; herein incorporated by reference in its entirety). High overlap between Master Regulators inferred from ARACNe analysis of completely independent Breast Cancer datasets was demonstrated. Thus, integration of target predictions from multiple datasets can improve the algorithm's performance without requiring data consolidation into a single dataset, which invariably introduces artifacts due to dataset specific bias.

Consistent with their previously reported activity, Pearson correlation analysis shows that five of the top six MRs (Stat3, C/EBPβ/δ, bHLH-B2, Runx1, and FosL2) are mostly activators of their regulon genes and only one (ZNF238) is a suppressor (2, 23; each herein incorporated by reference in its entirety). This can further indicate their respective potential as oncogenes or tumor suppressors. Since both C/EBPβ and C/EBPδ were among the top inferred MRs and they are known to form stoichiometric homo and heterodimers, with identical DNA binding specificity and redundant transcriptional activity (79; herein incorporated by reference in its entirety), the term C/EBP generically will be used to indicate these transcriptional complexes. The FET p-values for the enrichment of the MGES genes in the ARACNe-inferred MR regulons are respectively: ρ_{FosL2}=3.5E 44, ρ_{ZNF238}=3.1E 31, ρ_{bHLH-B}2=3.0E 29, ρ_{Runx1}=7.8E 24, ρ_{Stat3}=1.2E 21, ρ_{C/EBPβ}=3.2E 15. Thus, candidate MR regulons are highly enriched in MGES genes. The regulons of the six TFs show highly significant overlap, indicating their potential role in the combinatorial regulation of the MGES. Since TFs' expression is correlated, FET cannot compute statistical significance of this overlap. Significance was thus computed by comparing regulon overlap of each MR-pair against that of random TF-pairs with equivalent Mutual Information. Table I shows number of shared targets (lower left triangle) and p-value of regulon overlap (upper right triangle). For the TF pairs, the intersection between their regulon and the MGES is highly significant. This'further supports the role of these genes in a combinatorial Master Regulator Module (MRM), which controls the MGES program of GBM.

TABLE 1
Intersect between TFs and ARACNe targets (mesenchymal genes). The
number of mesenchymal genes shared as first neighbor by each pair of TFs
is reported on the lower left of the table. The statistical significance of
the target overlap for each pair of TFs after correction for the correlation
of the pair is shown on the upper right side of the table. The reported
P-values are test of independence between two TFs' neighborhoods
considering Mutual Information between TFs' gene expression profiles.

| TF | BHLHB2 | CEBPB | FOSL2 | RUNX1 | STAT3 | ZNF238 |
|---|---|---|---|---|---|---|
| BHLHB2 | 30 | 0.0e+00 | 0.0e+00 | 1.7e−03 | 2.5e−02 | 5.7e−03 |
| CEBPB | 12 | 20 | 0.0e+00 | 0.0e+00 | 5.2e−03 | 0.0e+00 |
| FOSL2 | 23 | 18 | 48 | 0.0e+00 | 0.0e+00 | 0.0e+00 |
| RUNX1 | 16 | 16 | 29 | 42 | 0.0e+00 | 0.0e+00 |
| STAT3 | 10 | 9 | 20 | 21 | 30 | 0.0e+00 |
| ZNF238 | 13 | 14 | 27 | 26 | 25 | 39 |

### Number of MES Targets

Alternative and Complementary MRA Analysis Tools.

Stepwise Linear Regression (SLR) was used to construct quantitative, albeit simplified MGES transcriptional regulation models (i.e. regulatory programs). In such models, log-expression of MGES genes is computed as a linear function of the log-expression of a few TFs (14, 96; each herein incorporated by reference in its entirety). Specifically, log 2 expression of the i-th MGES gene is the response variable and the log 2 expression of the TFs are the explanatory variables in the linear model log 2 xi=Σαᵢⱼ log₂ fⱼ+βᵢⱼ (96). Here, fⱼ represents the expression of the j-th TF in the model and the (αᵢⱼ, βᵢⱼ) are linear coefficients computed by standard regression analysis. TFs were iteratively added to the model, by choosing the one yielding the smallest relative error E=Σ|xᵢ−xᵢ₀|/xᵢ₀ between predicted and observed target expression. This was repeated until the decrease in relative error was no longer statistically significant, thus effectively preventing overfitting. TFs were chosen only among the 55 MRA-inferred MRs and TFs whose DNA binding signature was highly enriched in the proximal promoter of MGES genes and with a coefficient of variation (CV≧0.5), indicating a reasonable expression range in the dataset. This significantly reduces the number of candidate TFs. TFs were ranked based on the number of MGES target programs they affected. Surprisingly, the top six MRA-inferred TFs were among the top eight SLR-inferred TFs, showing significant robustness and consistency of the methods. The three TFs with the highest average coupling coefficients ( α̅ᵢ=Σᵢαᵢⱼ) were C/EBP (αᵢ=0.42), bHLH-B2 (αᵢ=0.41), and Stat3 (αᵢ=0.40), further indicating their potential role as MRs, with the next strongest modulator, ZNF238, showing a negative coefficient (αᵢ=−0.34) indicating its role as a transcriptional repressor.

Analysis of Candidate MRs in Human Glioma.

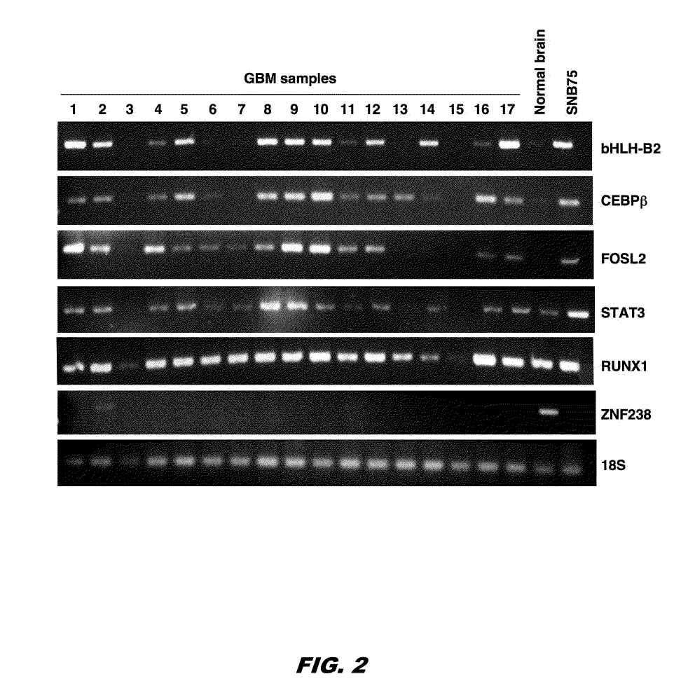

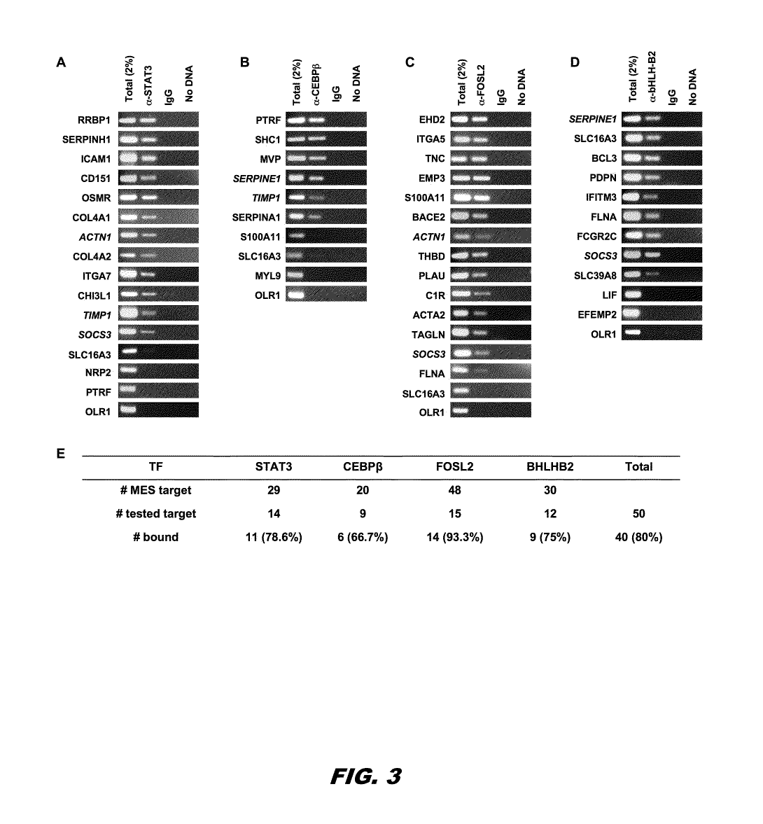

To analyze the expression patterns of the six candidate MRs, semi-quantitative RT-PCR was used in an independent set of 17 primary malignant gliomas. The analysis included both normal human brain and the glioma cell line SNB75 whose expression profile correlates with the mesenchymal centroid. bHLH-B2, C/EBPβ, FosL2, Stat3 and Runx1 were expressed in the SNB75 cell line. Expression of each of these TFs was present and concordant in at least 9 of 17 tumor samples (FIG. 2). This is in agreement with the reported incidence of malignant glioma with a mesenchymal phenotype (−50%) (77; herein incorporated by reference in its entirety). The Runx1 transcript was almost uniform in tumor samples and was also detectable in normal brain. Importantly, bHLH-B2, C/EBPβ and FosL2 transcripts were absent in normal brain, thus indicating a possible specific role of these TFs in gliomagenesis and/or progression. Stat3 levels were higher in GBM samples carrying high expression of bHLH-B2, C/EBPβ and FosL2. Conversely, expression of ZNF238 was readily detectable in normal brain but absent in SNB75 cells and in primary gliomas with the exception of one sample (#2) that displayed minimal expression levels (FIG. 2). This finding is consistent with the notion that the ability of ZNF238 to function as repressor of the MGES confers to the ZNF238 gene a tumor suppressor activity that is invariably abrogated in malignant glioma.

Biochemical Validation of MR Binding Sites.

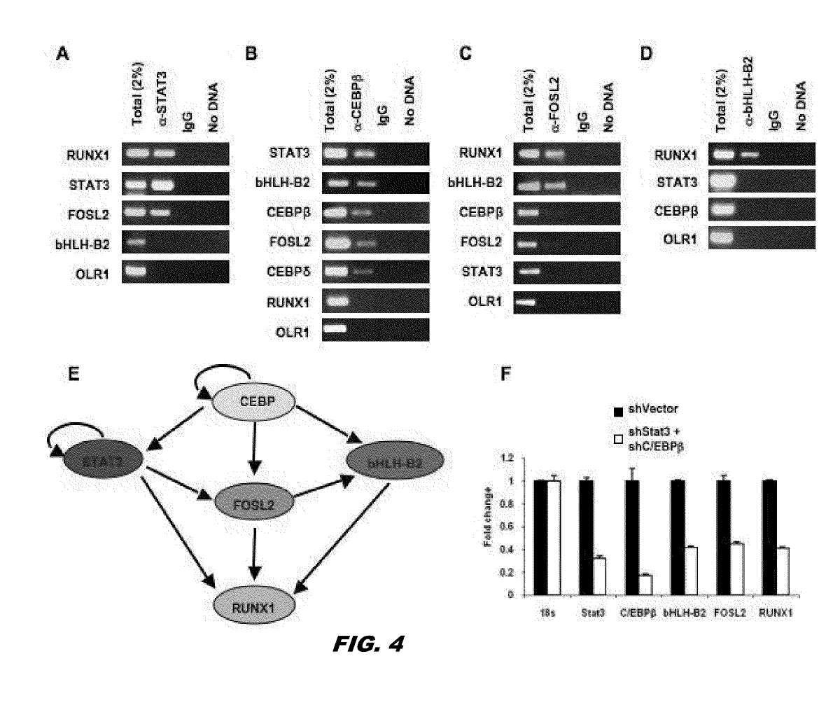

Each candidate MR was tested for its ability to bind to the promoter region (proximal regulatory DNA) of its predicted MGES targets. The target promoters were first analyzed in silico to identify putative binding sites. Promoter analysis was performed using the MatInspector software (www.genomatix.de; herein incorporated by reference in its entirety). A sequence of 2 kb upstream and 2 kb downstream from the transcription start site was analyzed for the presence of putative binding sites for each MR. ChIP assays were then performed near the best predicted site for each MR-target in the human glioma cell line SNB75, to validate targets of Stat3, bHLH-B2, C/EBPβ and FosL2, for which appropriate reagents were available. On average, 80% of the tested genomic regions can be immunoprecipitated by MR-specific antibodies but not by control antibodies (FIG. 3). Since binding can be co-factor mediated or occur in other promoter regions, this constitutes a lower-bound on the percent of MR-bound MGES genes. One can conclude that ARACNe accurately recapitulates the transcriptional activity of Stat3, bHLH-B2, C/EBPβ and FosL2 on the MGES genes in malignant gliomas.

Candidate MRs Form a Highly Connected and Hierarchically Organized Master Regulator Module.

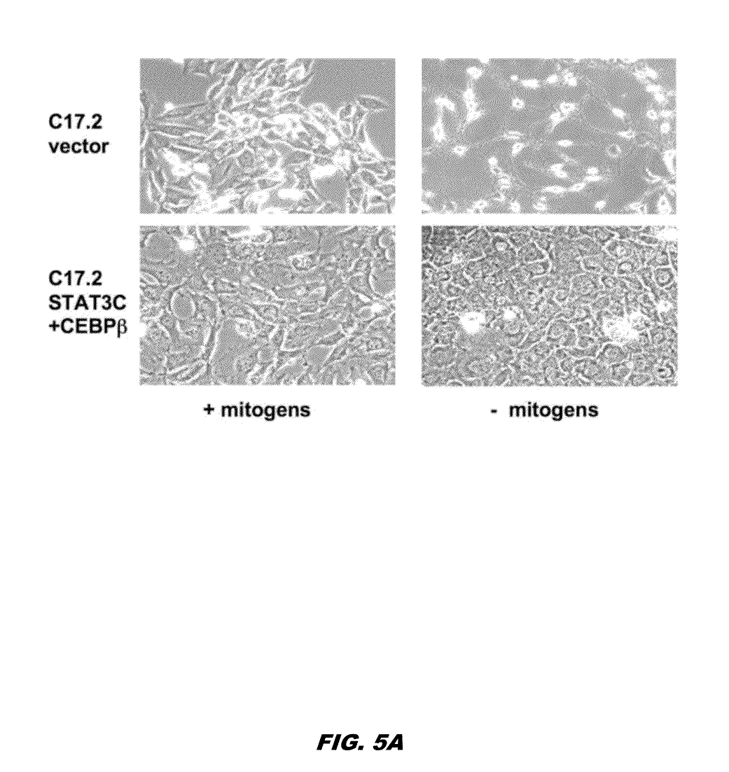

From recent results in yeast and mammalian cells, MRs of key cellular processes (a) are involved in auto-regulatory (AR), feedback (FB), and feed-forward (FF) loops (44, 68; each herein incorporated by reference in its entirety), (b) participate in highly interconnected TF modules (12; herein incorporated by reference in its entirety), and (c) are organized within hierarchical control structures (108; herein incorporated by reference in its entirety). Thus, whether the topology of the five candidate MRs involved in positive control of the MGES displayed such properties was considered. ChIP assays revealed that Stat3 and C/EBP occupy their own promoter and are thus involved in AR loops (FIGS. 4A-B). Additionally, Stat3 occupies the FosL2 and Runx1 promoters; C/EBPβ occupies those of Stat3, FosL2, bHLH-B2, C/EBPβ, and C/EBPδ (the latter two confirm the redundant autoregulatory activity of the two C/EBP subunits, FIG. 4B) (65, 79; each herein incorporated by reference in its entirety); FosL2 occupies those of Runx1 and bHLH-B2 (FIG. 4C); finally bHLH-B2 occupies only that of Runx1 (FIG. 4D). The regulatory topology emerging from promoter occupancy analysis is thus highly interconnected (12/15 possible interactions are implemented), has a hierarchical structure and is very rich in FF loops (FIG. 4E). The large number of FF loops can contribute to lower the MGES program sensitivity to short, random fluctuations (37; herein incorporated by reference in its entirety). Stat3 and C/EBP, which are also involved in AR and FF loops with a large fraction of MGES genes, appear to be at the top of the hierarchy. Lentivirus-mediated shRNA silencing of Stat3 and C/EBPβ in human GBM-derived stem-like cells (GBM-BTSCs) led to downregulation of the other TFs, confirming the hierarchical MRM organization (FIG. 4F). Without being bound by theory, (a) at least five of the six MRs participate in a hierarchical MRM control structure and (b) Stat3 and C/EBP can be master initiators and regulators of the mesenchymal signature of malignant gliomas.

Combined Expression of C/EBPβ and Stat3 Prevents Neuronal Differentiation and Induces Mesenchymal and Oncogenic Transformation of NSCs.

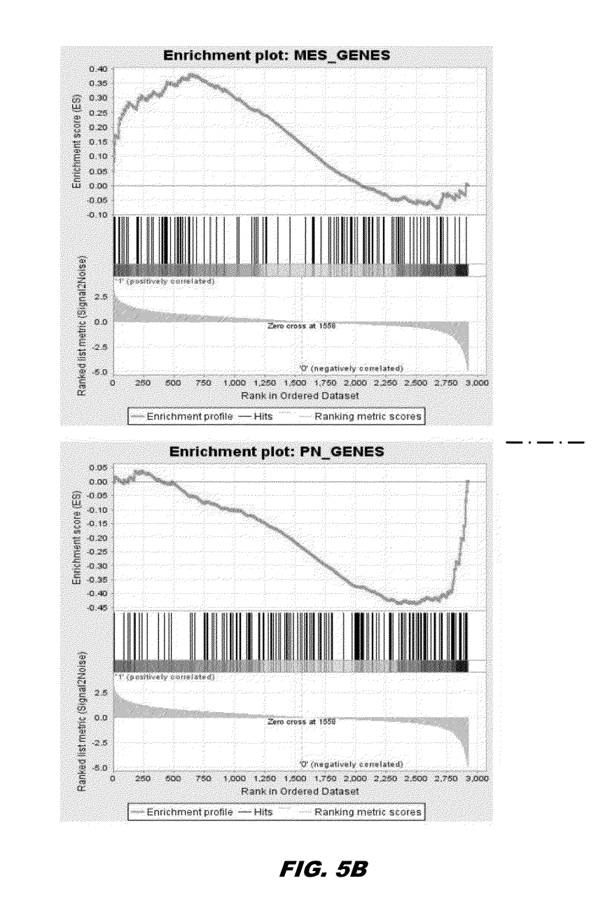

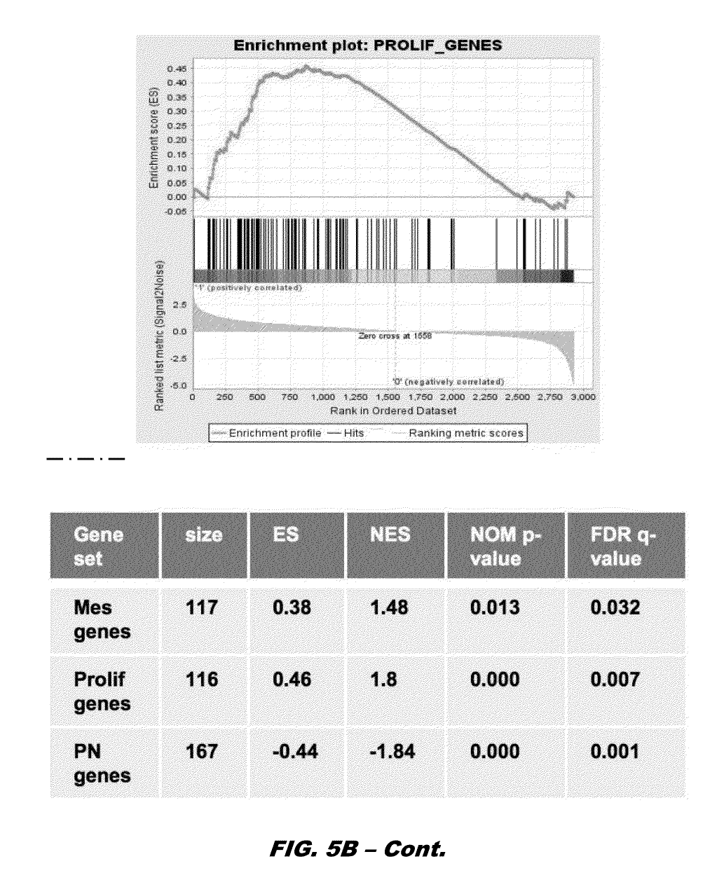

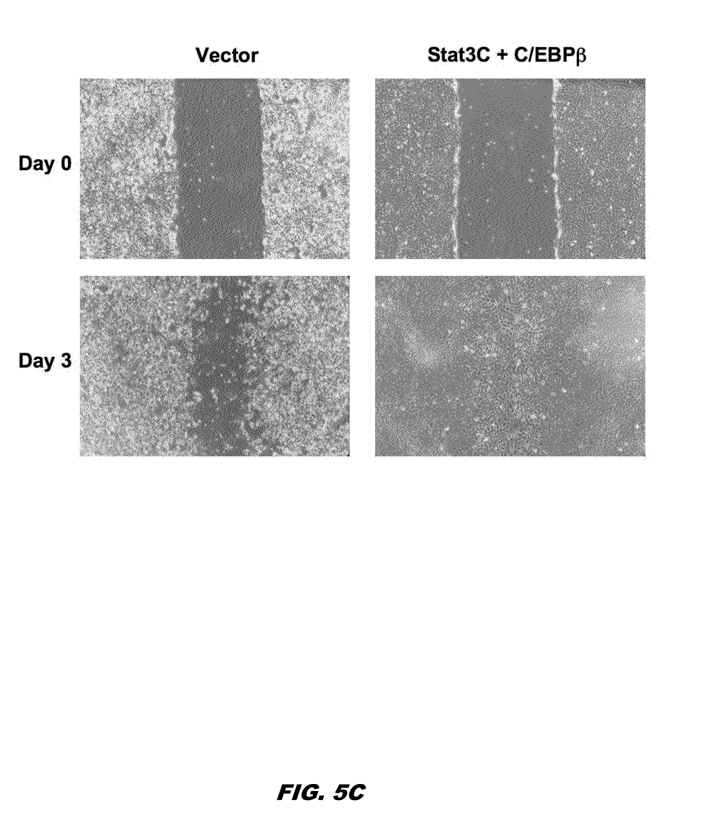

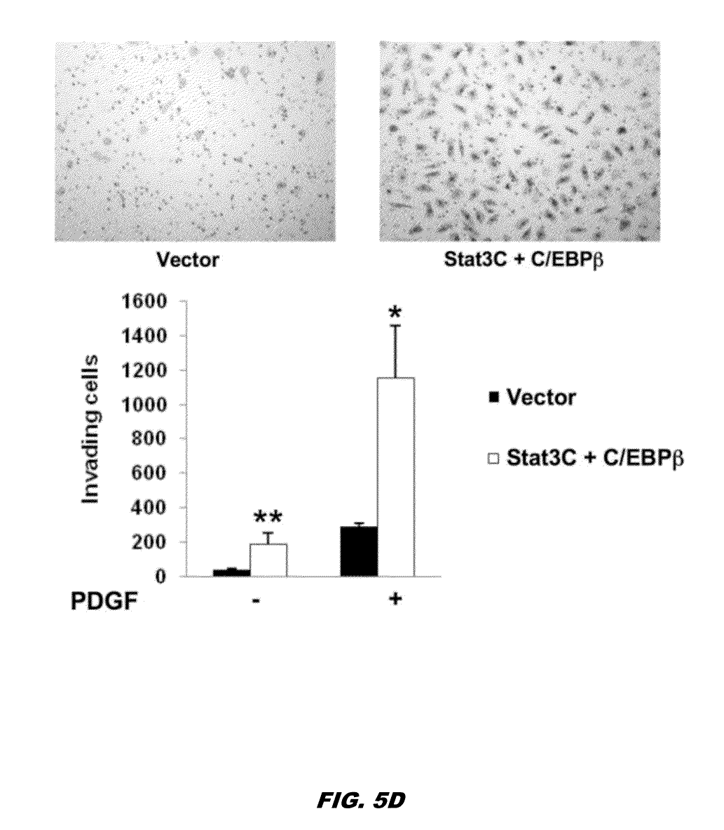

Without being bound by theory, NSCs are the cell of origin for malignant gliomas in the mesenchymal subgroup (77; herein incorporated by reference in its entirety). However, whether mesenchymal transformation in glial tumors recapitulates a normal albeit rare cell fate determination event intrinsic to NSCs remains unknown (95, 98, 105; each herein incorporated by reference in its entirety). Whether combined expression of Stat3 and C/EBPβ in NSCs is sufficient to initiate mesenchymal gene expression and to trigger the mesenchymal properties that characterize high-grade glioma was considered. An early passage of the stable, clonal population of mouse NSCs known as C17.2 was used because its enhanced yet constitutively self-regulated expression of sternness genes permits its cells to be efficiently grown as undifferentiated monolayers in sufficiently large, homogeneous and viable quantities to ensure reproducible patterns of self-renewal and differentiation without ever behaving in a tumorigenic fashion in vitro or in vivo (43, 72, 74; each herein incorporated by reference in its entirety). Following ectopic expression of C/EBPβ and a constitutively active form of Stat3 (Stat3C, 13; herein incorporated by reference in its entirety), dramatic morphologic changes of NSCs were observed, consistent with loss of ability to differentiate along the neuronal lineage (FIG. 5A). Parental and vector-transfected NSCs have the classical spindle-shaped morphology that is associated with the neural stem/progenitor cell phenotype. When grown in the absence of mitogens, these cells display efficient neuronal differentiation characterized by formation of a neuritic network (FIG. 5A, top-right panel). Conversely, expression of C/EBPβ and Stat3C leads to cellular flattening and manifestation of a fibroblast-like morphology. Remarkably, depletion of mitogens resulted in additional flattening with complete loss of every neuronal trait (FIG. 5A, bottom-right panel). These results indicate that expression of C/EBPβ and Stat3C efficiently suppresses differentiation along the neuronal lineage and induces mesenchymal features.

Next, whether C/EBPβ and Stat3C induce expression of the respective targets predicted by ARACNe and, more importantly, whether the induced expression pattern is consistent with that of the global MGES was considered. mRNA was extracted from duplicate samples of two independent C/EBPβ/Stat3C expressing and control clones of NSCs and hybridized custom expression arrays (Agilent Technologies) containing probes for 6,308 glioma-specific mouse and human genes. The Gene Set Enrichment Analysis method (GSEA) (92; herein incorporated by reference in its entirety) was used to test the enrichment of the mesenchymal, proliferative and proneural signatures (77; herein incorporated by reference in its entirety) among differentially expressed genes in C/EBPβ/Stat3C-expressing versus control cells. In this method, the Kolmogorov-Smirnoff test is used to determine whether two gene lists are statistically correlated. In this case, one list includes genes on the microarray expression profile dataset, ranked by their differential expression statistics across two conditions (e.g. ectopically expressed Stat3C-C/EBPβ vs. control), from most over- to most under-expressed. The other list contains non-ranked genes in a specific signature (e.g. mesenchymal). This is very useful to detect, for instance, situations where signature genes can be differentially expressed as a whole, even though the fold-change can be small for each gene in isolation. In this case, a gene-by-gene test, such as a T-test, can not be able to reveal statistical significance. The algorithm was set to implement weighted scoring scheme and the enrichment score significance is assessed by 1,000 permutation tests to compute the enrichment p-value. The analysis demonstrated that the global mesenchymal and proliferative signatures are both highly enriched in genes that are overexpressed in C/EBPβ/Stat3C-expressing NSCs. Conversely, the proneural signature is enriched in genes that are underexpressed in these cells (FIG. 5B). A subset of Stat3 and C/EBPβ targets of the microarray results was validated by quantitative RT-PCR (qRT-PCR).

Next, whether activation of the MGES by Stat3 and C/EBPβ is sufficient to transform NSCs into cells that can efficiently migrate and invade was considered. Two assays were used to address this question. The first (“wound assay”) evaluates the ability to migrate and fill a scratch introduced in cultures of adherent cells (FIG. 5C). The second (“Matrigel invasion assay”) tests how cells invade a Boyden chamber filter coated with a physiologic mixture of extracellular matrix components and concentrate the lower side of the filter (FIG. 5D). When the two assays were performed on C/EBPβ/Stat3C-expressing and control NSCs clones, it was found that the expression of the two TFs robustly promoted migration and invasion through the extracellular matrix (FIGS. 5C-D). The effects of C/EBPβ and Stat3C on migration and invasion of NSCs were similar in the absence of mitogens or in the presence of PDGF (FIG. 5D). Similarly, ectopic bHLH-B2 was irrelevant for the MGES and phenotypic behavior of Stat3C-C/EBPβ-expressing NSCs.

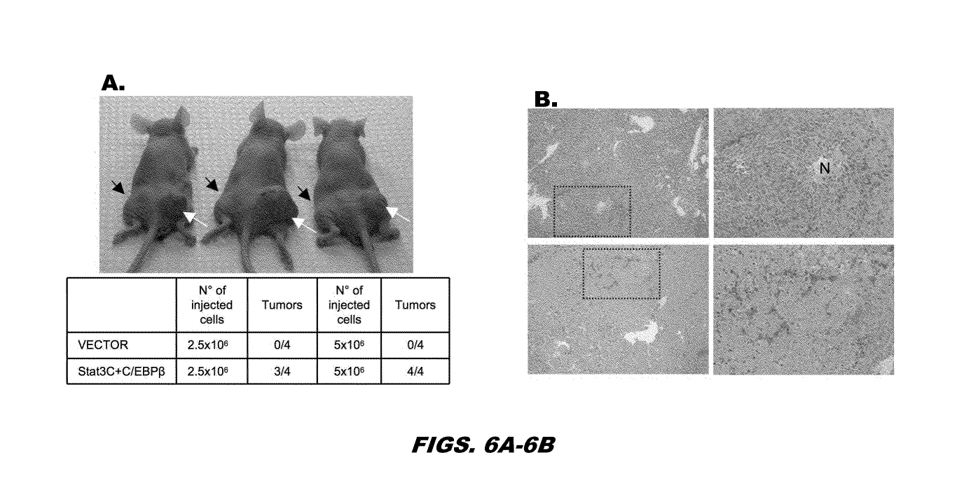

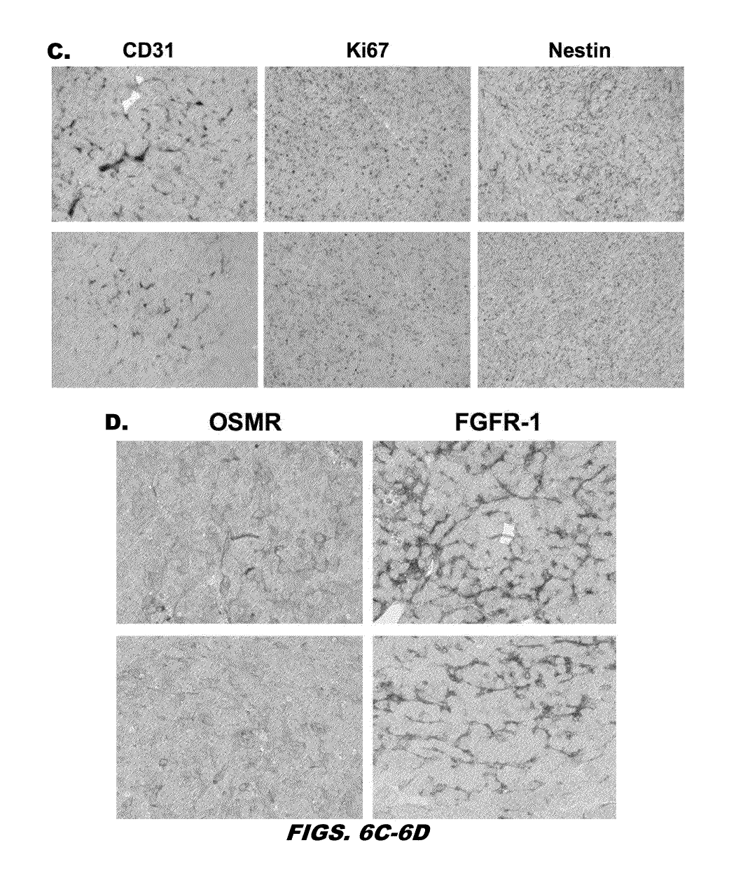

To ask whether Stat3 and C/EBPβ confer tumorigenic potential to NSCs in vivo, sub-cutaneous heterotopic transplantation of C17.2-Stat3C-C/EBPβ (or empty vector as control) was used. C17.2-Stat3C/C/EBPβ cells developed fast-growing tumors with high efficiency (4 out of 4 mice in the group injected with 5×10⁶ cells and 3 out of 4 mice in the group injected with 2.5×10⁶ cells), whereas NSCs transduced with empty vector never formed tumors (FIG. 6A). Histological analysis demonstrated that the tumors resembled human high grade glioma, exhibited large areas of polymorphic cells, had tendency to form pseudopalisades with central necrosis and although injected in the flank, a low angiogenic site, displayed extensive vascular proliferation, as confirmed by immunostaining for the endothelial marker CD31 (FIGS. 6B-C). Proliferation in the tumors was very high as determined by reactivity for Ki67. In line with the presence of stem-like cells, human GBM regularly exhibit expression of primitive markers. Corroborating this, it was found that the tumors stained positive for the progenitor marker nestin (FIG. 6C). Finally, positive immunostaining for the mesenchymal signature proteins OSMR and the FGF receptor-1 (FGFR-1) indicated that oncogenic transformation of neural stem cells had occurred in the context of reprogramming towards the mesenchymal lineage (FIG. 6D). Together, these findings establish that introduction of the two MRs of MGES in NSCs not only induces expression of the entire MGES but is also sufficient to transduce to these cells the key phenotypic characteristics of glioma aggressiveness that have been previously associated with that signature.

Stat3 and C/EBPβ are Essential for Expression of the MGES and Aggressiveness of Human Glioma Cells and Primary Tumors.

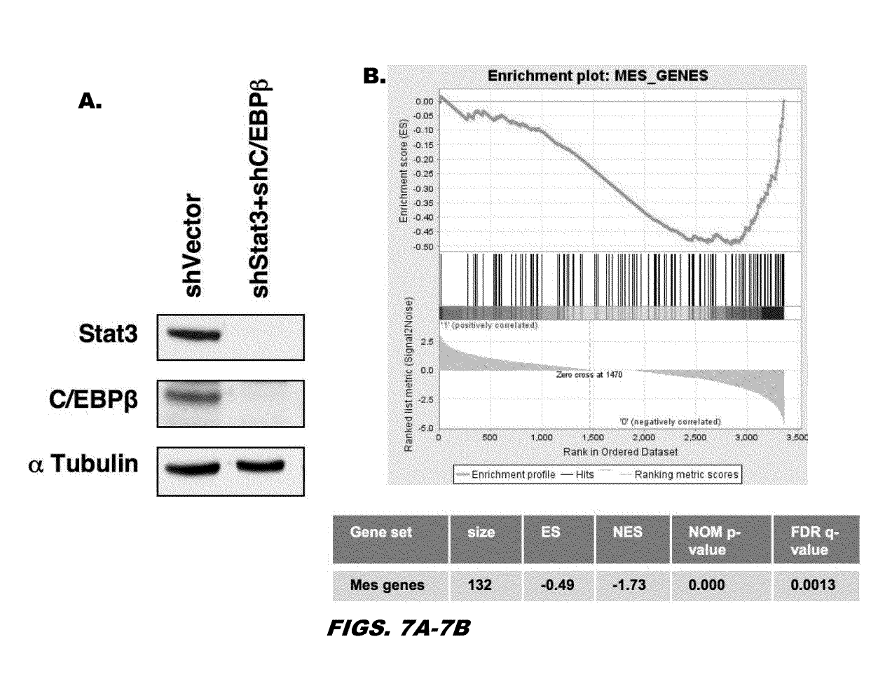

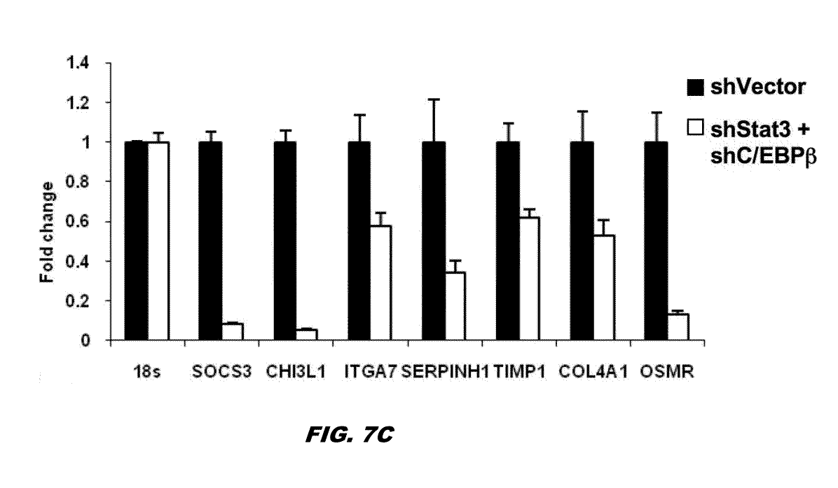

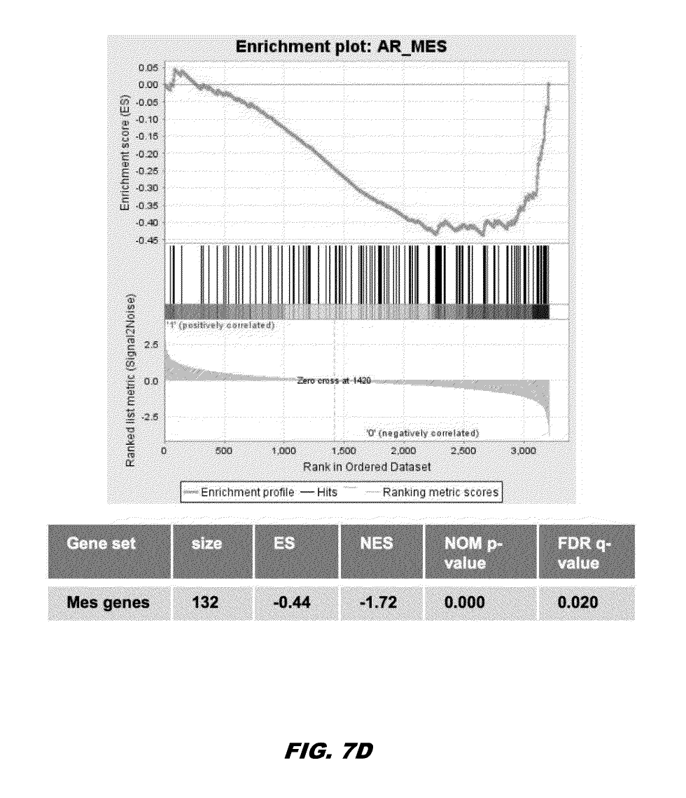

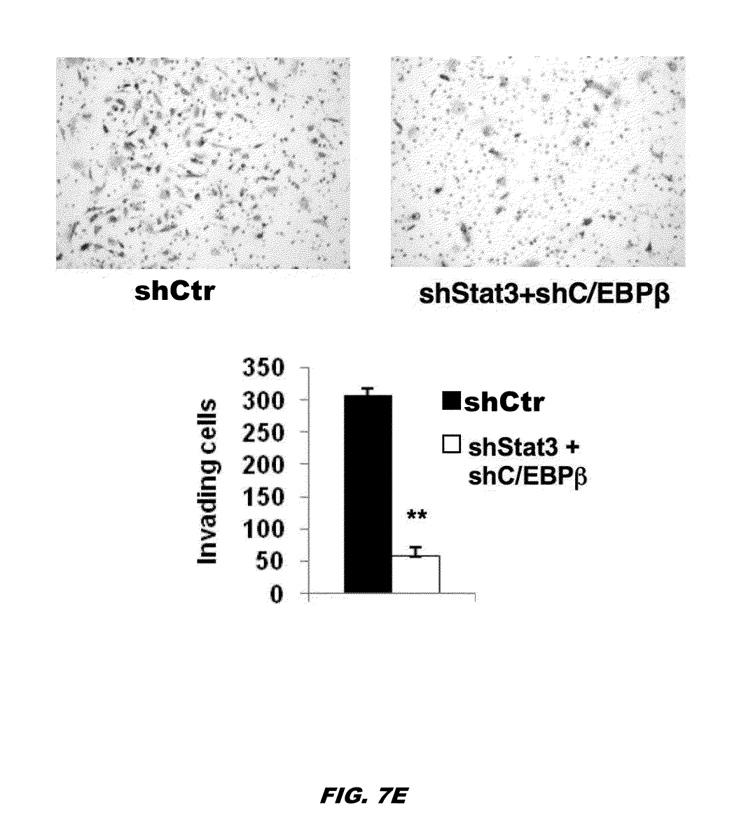

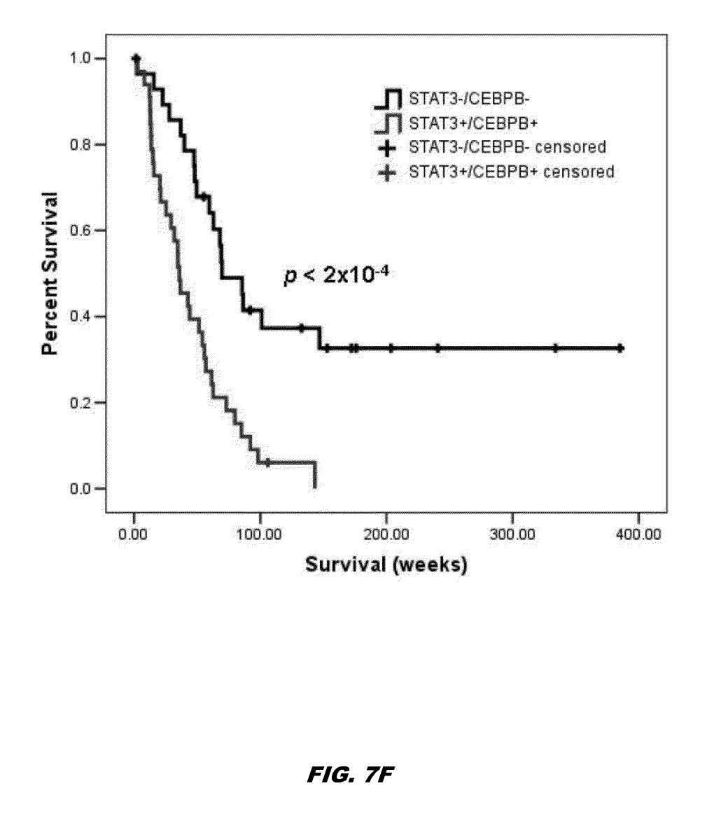

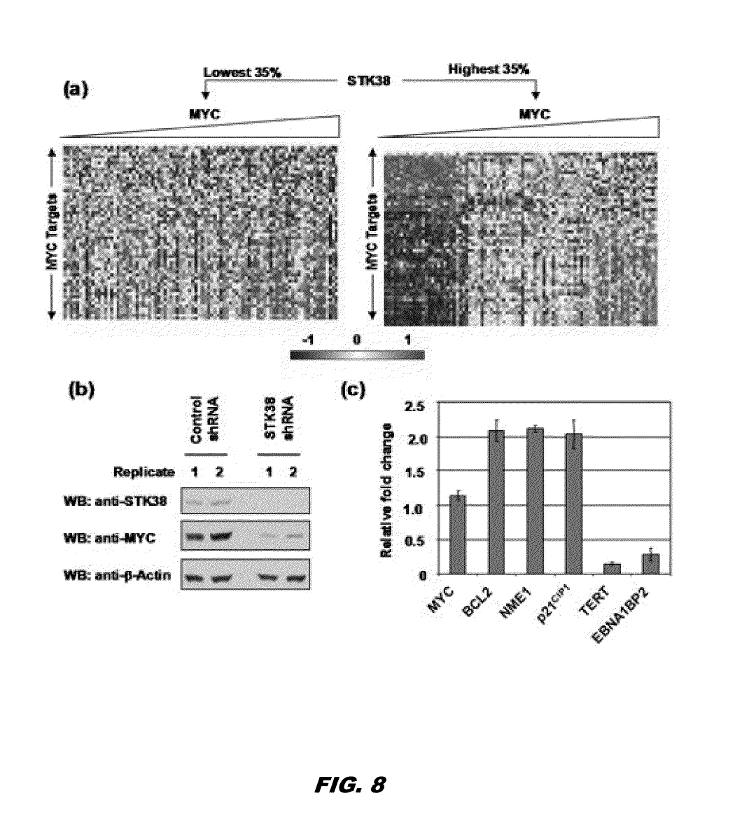

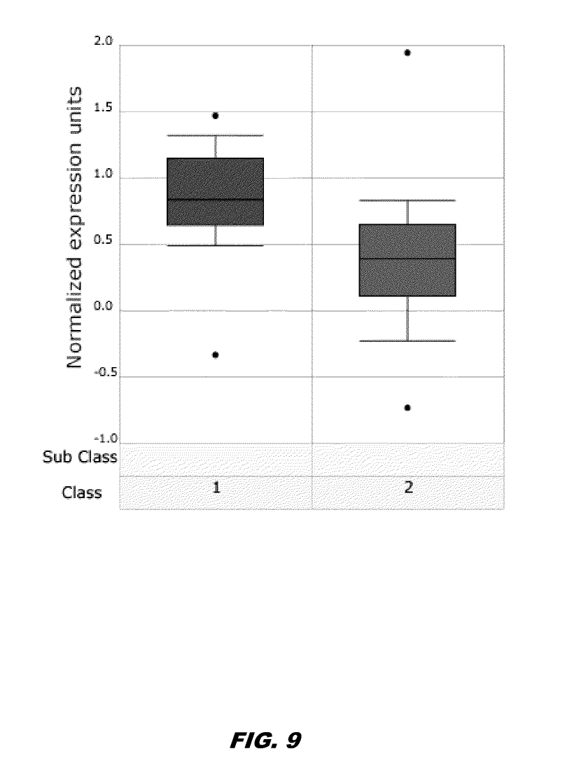

To assess the significance of constitutive Stat3 and C/EBPβ in cells responsible for glioma tumor growth in humans, it was sought to abolish the expression of Stat3 and C/EBPβ in GBM-derived brain tumor stem-like cells that closely mimic the biology of the parental primary tumors and retain tumor-initiating capacity (GBM-BTSCs, 42; herein incorporated by reference in its entirety). Transduction of GBM-BTSCs with specific shRNA-carrying lentiviruses efficiently silenced endogenous Stat3 and C/EBPβ (FIG. 7A). Expression analysis using GSEA and qRT-PCR showed that depletion of Stat3 and C/EBPβ in GBM-BTSCs dramatically suppressed expression of the MGES genes (FIGS. 7B-C). Next, the “mesenchymal” human glioma cell line SNB19 was infected with shStat3 and shC/EBPβ lentiviruses and confirmed that silencing of Stat3 and C/EBPβ depleted the mesenchymal signature even in established glioma cell lines (FIG. 7D). Furthermore, silencing of the two TFs in SNB19 eliminated 80% of their ability to invade through Matrigel (FIG. 7E).

As final test for the mesenchymal regulatory role of Stat3 and C/EBPβ in human glioma, an immunohistochemical analysis for C/EBPβ and active, phospho-Stat3 in human tumor specimens was conducted and compared the expression of these TFs with YKL-40 (a well-established mesenchymal protein also known as CHI3L1, Refs. 66, 75; each herein incorporated by reference in its entirety) as well as patient outcome in a collection of 62 newly diagnosed GBMs. FET showed that expression of either C/EBPβ and Stat3 were significantly correlated with YKL-40 expression (C/EBPβ, p=4.9×10⁻⁵; Stat3, p=2.2×10⁻⁴). However, the correlation was higher when double positive tumors (C/EBPβ+/Stat3+) were compared to double negatives (C/EBPβ−/Stat3−, p=2.7×10⁻⁶). Furthermore, double positive tumors were associated with markedly worse clinical outcome than tumors that were either single or double negatives (log-rank test, p=0.0002, FIG. 7F). Positivity for either of the two TFs remained predictive of negative outcome but with lower statistical strength than double positivity (C/EBPβ, p=0.0022; Stat3, p=0.0017). These results provide compelling indication that the synergistic activation of C/EBPβ and Stat3 generates mesenchymal properties and marks the poorest survival in patients with GBM.

Computational Inference of MR Modulators.

MINDy is the first algorithm for the systematic identification of post-translational modulators of TF activity (100, 101; each herein incorporated by reference in its entirety). It identifies candidate TF-modulators by testing whether, given the expression of a putative modulator gene, the Conditional Mutual Information (CMI) I[TF; t|M] between a TF and one of its targets changes as a function of the availability of M. In Ref. 102 (herein incorporated by reference in its entirety), four modulators of the MYC TF in human B cells, including the STK38 kinase, the HDACl histone deacetylase, and two co-TF factors, bHLH-B2 and MEF2B were biochemically validated. FIG. 8 shows experimental data supporting the role of STK38 as a post-translational modulator of MYC activity. Experimental evidence for the other validated modulators is provided in the appendix (102; herein incorporated by reference in its entirety). In Ref. 100 (herein incorporated by reference in its entirety), MINDy analysis was extended to systematically reverse-engineer the interface between ˜800 signaling proteins (including protein kinases, phosphatases, and cell surface receptors) and an equivalent number of TFs expressed in human B cells. STK38 was experimentally validated as a pleiotropic serine-threonine kinase, affecting not just MYC but several other TFs. Thus, MINDy is able to identify post-translational modulators of transcriptional programs. For details on MINDy implementation, see Refs 55, 100; each herein incorporated by reference in its entirety.

MINDy's applicability has been significantly enhanced by the availability of a large set of microarray expression profile for high grade glioma from The Genome Cancer ATLAS/TCGA effort. This dataset is now equivalent in statistical power to the human B cell dataset used for the development of the MINDy approach. As discussed herein, the new MINDy analysis of Stat3 modulators recapitulates the major direct and pathway mediated modulators of Stat3 activity and demonstrates the feasibility of the MINDy algorithm. In Ref. 55 (herein incorporated by reference in its entirety), it was shown that MINDy outputs were able to build a genome-wide interactome and to infer both causal oncogenic lesions as well as mechanism of action of specific chemical perturbations. Furthermore, in Ref. 100 (herein incorporated by reference in its entirety), the complete and biochemically validated analysis of the interface between signaling proteins and TFs in human B cells was reported. Results from the latter, as also shown in FIG. 8, have allowed the computational identification of kinases silenced by lentivirus-mediated transduction of shRNA constructs in human B cell, using only transcriptional data.

A key requirement of the algorithm is the availability of ≧200 GEPs, so that the Conditional MI dependency on the modulator can be accurately measured. False negatives further improve with higher sample sizes (i.e. fewer modulators are missed). Studies were limited by a sample size that was too small to be effective (176 samples). However, a set of 236 GBM-related GEPs was recently made available by the ATLAS/TCGA project (1). Using this larger dataset sufficient statistical power was achieved to infer several post-translational modulators of Stat3 and C/EBPβ activity. MINDy-inferred modulators can be used for two independent goals. First, preliminary analysis of gene copy number (GCN) alterations from matched TCGA samples revealed that several genes encoding Stat3 and C/EBPβ modulators harbor genetic alterations in high-grade glioma, supporting their potential tumorigenic role (Table 2).

TABLE 2
Summary table of the post-translational modulators of STAT3 and
CEBPβ identified by MINDy in two separate analyses. Shown are TF and signaling
modulators, having significant copy number aberrations enrichment in patients with high
expression of YKL40, selected as marker gene. Patients were binned into three categories,
high, medium and low, according to the YKL40 expression level. Modulators are called
significant whenever there is an enrichment in the frequency of patients for the corresponding
aberration with a p-value of the χ2 < 5% and are sorted left to right by decreasing number of affected targets.

| Source Analysis | TF | | | | | | | | |
|---|---|---|---|---|---|---|---|---|---|
| Fsym | STAT3 | | | | | CEBPB | | | |
| Cytoband | 7q31.2 | 10p11.21 | 17q21 | 7q32 | 22q12.2 | 19q13.31 | 10p12 | 17q21 | 10p12 |
| Modulator | TFEC | CREM | RARA | IRF5 | PATZ1 | ZNF576 | MSRB2 | RARA | MLLT10 |
| YKL40 high | | | | | | | | | |
| expression | | | | | | | | | |
| levels | | | | | | | | | |
| Enrichment in | Yes | No | No | Yes | No | Yes | No | No | No |
| Amplifications | | | | | | | | | |
| Enrichment in | No | Yes | Yes | No | Yes | No | Yes | Yes | Yes |
| Deletions | | | | | | | | | |
| Lowest χ | 1.43% | 0.46% | 4.55% | 2.18% | 3.39% | 2.53% | 0.46% | 4.55% | 0.46% |
| Significant | Amplifica | Deletion | Deletion | Amplifica | Deletion | Amplifica | Deletion | Deletion | Deletion |
| Alteration | | | | | | | | | |

| Source Analysis | Signaling | | | | | | |
|---|---|---|---|---|---|---|---|
| Fsym | STAT3 | | | CEBPB | | | |
| Cytoband | 10q26 | 7q22-q31.1 | 19q13.3 | 7p12 | 10q26 | 19q13 | 22q12\|7p14.3-p14.1 |
| Modulator | FGFR2 | SRPK2 | PRKD2 | EGFR | FGFR2 | VRK3 | CAMK2B |
| YKL40 high | | | | | | | |
| expression | | | | | | | |
| levels | | | | | | | |
| Enrichment in | No | Yes | Yes | Yes | No | Yes | Yes |
| Amplifications | | | | | | | |
| Enrichment in | Yes | No | No | No | Yes | No | No |
| Deletions | | | | | | | |
| Lowest χ | 4.12% | 1.05% | 4.55% | 0.67% | 4.12% | 4.55% | 0.47% |
| Significant | Deletion | Amplifica | Amplification | Amplification | Deletion | Amplifica | Amplification |
| Alteration | | | | | | | |

This is important because the Stat3 and C/EBPβ loci are not direct targets of genetic alterations in GBM. Hence, one can predict that genetic alterations can target their upstream regulators. Specifically, several GCN alterations of Stat3 and C/EBPβ modulators co-segregate with overexpression of YKL40, a marker of MGES activation. Without being bound by theory, genetic alterations of the modulator genes can irreversibly activate these MRs, thus leading to constitutive activation of the MGES in high-grade glioma. Second, the modulator proteins can constitute appropriate drug targets for therapeutic intervention.

The inferred repertoire of Stat3 modulators was compared to literature data (21, 34; each herein incorporated by reference in its entirety). The analysis revealed that several inferred modulators are known to regulate Stat3 activity post-translationally, either by direct physical interaction, or by effecting well-characterized pathways known to affect Stat3 function, mostly through phsphorylation cascades. Among the putative Stat3 modulators, we found the β2 adrenergic receptor (ADRB2) and Src kinase Lyn, which mediate phosphorylation and activation of Stat3 (103, 107; each herein incorporated by reference in its entirety). Conversely, the cdk2 and GSK3β kinases and the tumor suppressor PTEN are negative regulators of Stat3 phosphorylation and activity (10, 90, 93; each herein incorporated by reference in its entirety). Our approach was also able to identify the α subunit of Protein Kinase C(PRKCA), the MAP kinase MEK2 (MAP2K2) and the Receptor 2 for FGF (FGFR2), three essential components of signaling pathways known to modulate Stat3 activity (28, 39, 71, 73; each herein incorporated by reference in its entirety). Finally, MINDy identified Dyrk2 as a Stat3 modulator and, in screening assays Dyrk kinases have emerged as phosphorylation kinases for Stat3 (60; herein incorporated by reference in its entirety). These findings mirror those obtained for MYC (101, 102; each herein incorporated by reference in its entirety) and indicate that MINDy is effective in the identification of post-translational modulators of MR activity.

Conclusions.

Computational, ChIP and functional experiments, motivated by the inferred network topology, showed that Stat3 and C/EBP are key MRs of the MGES. However, the participation of the transcriptional repressor ZNF238 as a principal negative regulator of the mesenchymal signature, combined with the invariable loss of expression of ZNF238 in primary GBM, indicate that the full manifestation of the MGES inevitably requires elimination of the constraints imposed by ZNF238. Initial results will be followed up with a comprehensive computational reconstruction of the transcriptional and post-translational interactions that structure the regulatory network driving the MGES. The mechanisms used by glioma cells to silence the expression of ZNF238 will also be determined and tested whether this TF is a tumor suppressor gene in malignant brain tumors. Finally, computational approaches will be used to identify post-translational modulators of the ‘mesenchymal TFs’ and validate in vivo their functional activity and their value as therapeutic targets.

Future Directions.

As shown in this Example, use of tumor biopsy GEPs was sufficient to discover candidate synergistic oncogenes and tumor suppressor genes. However, the highly heterogeneous nature of the disease can prevent dissection of many TF-targets and upstream modulators. Given the decreasing cost of GEP microarrays and the availability of high-throughput robotic platforms available to us, a new, highly informative dataset will be assembled using a cellular context that is highly specific to the transformation under study. Specifically, a connectivity map (40; herein incorporated by reference in its entirety), using ˜200 chemical perturbations of human GBM-derived BTSCs will be produced. These cells represent the best cellular model for human GBM because they closely mimic the genotype, gene expression profile and in vivo biology of their parental primary tumor (42, 99; each herein incorporated by reference in its entirety).

Furthermore, it was shown that MGES expression in GBM-BTSCs requires the activity of the MRs Stat3 and C/EBPβ (FIG. 7). Therefore, GBM-BTSCs represent a model human cellular system to produce a glioma connectivity map and to study regulation of the MRs of mesenchymal signature GBM in vitro. This new dataset will be highly complementary to the GBM data produced by the TCGA project and is of critical importance to achieve the aims of this proposal. Specifically, while TCGA GEPs represent the natural physiologic variability of GBM samples and can be representative of a variety of diverse genetic and epigenetic abnormalities, the connectivity map will reflect the response of high-grade (mesenchymal) glioma to non-physiologic (i.e., chemical) perturbations. Thus, the combination of the two resources will allow optimal dissection of both type of processes.

Compound Selection and Optimization.

˜200 compounds will be prioritized by analysis of MCF7, PC3, HL60, and SK-MEL5 connectivity map data (40; herein incorporated by reference in its entirety). Optimal compounds will be those producing the most informative profiles. Several methods can be used for this analysis, including Principal Component Analysis (PCA), unsupervised clustering, and greedy optimization techniques to select maximum-entropy GEP subsets, among others. The Genome wide 44Kx12 Illumina array (HumanHT-12 Expression BeadChip) supports analysis of ˜200 assays (in replicate) and appropriate controls for approximately. As opposed to Ref. 40 (herein incorporated by reference in its entirety), where compounds were screened at a fixed 10 μM concentration in DMSO, the selected compounds will be profiled at multiple concentrations to determine optimal parameters for ˜10% growth inhibition of GBM-BTSCs, G110, after 48 h. This will optimize the screening, providing maximally informative data. Higher concentrations can produce largely equivalent cellular stress responses (e.g., apoptosis), while lower concentrations will produce little or no effects on cell dynamics.

Perturbation Assays and Microarray Expression Profiling.

GBM-BTSCs will be treated with selected compounds at G110 concentration in replicate, harvested after 6 h (to minimize secondary response effects), and profiled using the Illumina HumanHT-12 Expression BeadChip array. These monitor ˜44,000 probes covering known human alternative splice transcripts. Appropriate negative controls will be generated using the compound delivery medium (DMSO). Arrays will be hybridized and read by the Columbia Cancer Center genomic core facility. The lab has significant experience using the Illumina array, including automation and optimization of mRNA extraction and labeling protocols on the Hamilton Star microfluidic station. Since ARACNe requires >100 GEPs and MINDy requires >250 GEPs to achieve sufficient statistical power, the dataset (˜400 GEPs) is adequately powered to support both analyses. The resulting dataset will be referred to as the High-grade Glioma Connectivity Map (HGCM). Additionally, two public datasets will be analyzed including expression profiles from tumor samples (42, 77; each herein incorporated by reference in its entirety) as well as the 236 samples from the TCGA, identified respectively as HGEP_{Lee}, HGEP_{Ph}, and HGEP_{TCGA}.

### Example 3

### Creation of a High-Accuracy Map of Regulatory Interactions Effecting the MGES of High-Grade Glioma

In this example, the molecular interaction networks and transcriptional modules that regulate the mesenchymal phenotype of malignant glioma will be dissected, modeled, and interrogated. This phenotype, which displays a specific genetic signature identified by molecular profiling, is characterized by the activation of several genes involved in invasiveness and tumor angiogenesis and has been associated with a very poor prognosis. Genes causally involved in tumorigenesis or responsible for the aggressiveness of the malignant phenotype will be identified. Furthermore, computational tools will be designed and used to integrate the rich source of genetic, epigenetic, and functional data assembled by The Genome Cancer ATLAS/TCGA project on Glioblastoma Multiforme (GBM) to identify “druggable” proteins that can affect the mesenchymal phenotype, thus providing appropriate targets for therapeutic intervention (see EXAMPLES 5-7).

To find the Master Regulators of a malignant phenotype, the ARACNe algorithm, developed for the dissection of mammalian transcriptional networks and validated, will be coupled with new algorithms that model the regulatory process, by integrating DNA binding signatures. Preliminary studies (EXAMPLE 2) show that ARACNe identifies a small, tightly connected, self-regulating module comprising six transcription factors (TFs) that appears to regulate the mesenchymal signature of human high-grade glioma. This example discusses the reverse engineering and dissection of crucial mechanisms involved in the pathogenesis of GBM, one of the most lethal forms of human cancer.

A reverse engineering computational approach will be applied to dissect and validate the transcriptional network that drives the mesenchymal phenotype of high-grade glioma. The expression of mesenchymal and angiogenesis-associated genes in malignant human glioma is associated with very poor clinical outcome. ARACNe (Algorithm for the Reconstruction of Accurate Cellular Networks), one of the tools developed by the Columbia National Center for Biomedical Computing (MAGNet), has been used to identify transcription factors regulating a mesenchymal gene expression signature associated with poor prognosis. The latter was identified by hierarchical clustering of a wide collection of microarray expression profiles of malignant glioma.

The analysis has identified a highly interconnected module of six transcription factors that regulate each other as well as the vast majority of the mesenchymal genes. The computational analyses as also been extended to new algorithms able to predict post-translational modulators of the master transcriptional regulators (MINDy, Modulator Inference by Network Dynamics). New computational tools will be designed and used to integrate the many sources of genetic, epigenetic and functional date available on human brain tumors. The goals are: to reconstruct and experimentally manipulate the transcriptional and post-translational programs responsible for the expression of the mesenchymal signature of high-grade glioma (see EXAMPLE 2 and herein); to elucidate the mechanism by which high-grade glioma silence ZNF238, a transcriptional repressor of the mesenchymal signature, and test the role of ZNF238 gene inactivation in gliomagenesis in the mouse (EXAMPLE 4); to computationally identify and experimentally validate “druggable” genes that regulate the mesenchymal signature in malignant glioma and to test them as candidate therapeutic targets (EXAMPLE 5); to assemble and disseminate a genome-wide, Human Glioma interactome (HGi) that will integrate the diverse sources of genetic, epigenetic, and functional alterations that characterize the mesenchymal phenotype of high-grade glioma (EXAMPLE 6). The HGi will be accessible to the scientific community via the MAGNet Center dissemination infrastructure. Ultimately, the aim is to exploit the computationally inferred and experimentally validated regulators of glioma aggressiveness as invaluable new targets for therapeutic intervention.

Reconstruction of the Combinatorial Regulatory Program for the Expression of the Mesenchymal Signature of High-Grade Glioma and Phenotypic Analysis of its Disruption in GBM-BTSCs.

The goal of these experiments is the integration of the transcriptional network predicted by ARACNe, the post-translational interactions predicted by MINDy, the binding data generated by ChIP-on-Chip experiments, the proteomic TF-TF interaction experiments, and the expression profile analysis of the changes after inactivation of Stat3 and C/EBPβ TFs in GBM-BTSCs. By combining various data sources, the key proteins required for MGES activation and maintenance will be uncovered and a comprehensive view of the cellular network driving the MGES in malignant glioma will be provided.

ARACNe analysis. ARACNe will be used with 100 rounds of bootstrapping on each of four datasets (HGCM, HGEP_{Lee}, HGEP_{Ph}, and HGEP_{TCGA}) to generate comprehensive high-grade glioma transcriptional networks (58; herein incorporated by reference in its entirety). TFs will be identified based on their specific molecular function annotation in the Gene Ontology. The analysis protocol described in Ref. 58 (herein incorporated by reference in its entirety) will be followed to accomplish the following:

Stat3 and C/EBP Target Identification

An exhaustive set of candidate targets of the Stat3 and C/EBP TFs will be examined. The new and comprehensive set of targets will be used to further elucidate the role of these validated MRs in the direct and indirect control of mesenchymal genes and in the transformation of NSCs. TF-targets can have been missed due to the relatively small and heterogeneous sample dataset used to reconstruct the MGES control network shown in FIG. 1. Thus, integration of data from four datasets will significantly increase the statistical power and usefulness of the approach. Specifically, the HGCM will provide information on interactions driven by non-physiologic perturbations, while the other three sets will provide information about physiologic transcriptional response.

Identification of Additional Regulators of the MGES

Decreasing TF-target false negatives will greatly enhance our ability to infer additional MRs, whose regulons can have been too small to assess enrichment when computed from the Aldape dataset. Additional metrics, other than FET, will be explored to rank candidate MRs, including target density odds ratio, coefficient analysis from SLR (described in EXAMPLE 2) and GSEA (49; herein incorporated by reference in its entirety). These metrics are not affected by regulon size and will provide a more unbiased ranking than the FET. Inferred MRs will be assembled into the MGES Master Regulatory Module (MRM).

The identification of physical interactions between MRM TFs will provide us with valuable information to design further experimental validations for bioinformatics results. Although the detailed experimental plan will obviously depend on the nature of the interaction(s) that will be demonstrated, the interactions between two activator TFs can be required for full activation of the target mesenchymal promoters. Conversely, interaction(s) between an activator TF and a repressor TF can function to restrain the activity of the activator TF bound to the DNA regulatory region of the mesenchymal promoters. Both overexpression and silencing experiments will be appropriate to interrogate the consequences of TF-TF interactions for the expression of selected mesenchymal genes and/or the entire MGES

Identification of Upstream Regulators

Additional TFs that are candidate upstream transcriptional regulators of the MRM TFs will be identified. If both genes are TFs, ARACNe cannot determine directionality. Thus, additional assays and analysis can be necessary, such as the identification of DNA binding site and ChIP assays.

Full Transcriptional Regulation Mapping

A complete transcriptional network will be inferred using ARACNe, involving TFs that are expressed in the cells of interest (EXAMPLE 6).

Evidence from the four datasets, as well as additional evidence sources such as interaction databases, literature data, and interactions in orthologous organisms will be integrated (55; herein incorporated by reference in its entirety). The Bayesian evidence integration approach using either Naíve Bayes classifiers or a Bayesian Network approach will be used, depending on the statistical correlation of the clues originating from each dataset (see Ref. 55; herein incorporated by reference in its entirety). The approach involves the use of established machine learning methods. Additional integrative approaches, such as Adaboost (9; herein incorporated by reference in its entirety) will also be tested and compared to the Bayesian evidence integration approach. Based on prior work (36; herein incorporated by reference in its entirety) and B cell interactome data (55; herein incorporated by reference in its entirety), one can expect that clues arising from different GEP sets will not be statistically independent and that a Bayesian Network analysis can be needed. Positive and Negative Gold standards will be based on evidence in TRANSFAC, other Protein-DNA interaction databases, and ChIP assays. This will provide an ideal complement of evidence from both tumor samples (heterogeneous context) and from the HGCM GBM-BTSC connectivity map (homogeneous context), thus allowing an ideal integration of TF-targets responding under diverse physiological and perturbation related stimuli.

MINDy Analysis.

MINDy (see EXAMPLE 2) will be used on the two datasets of sufficient size (HGCM and HGEPTCGA) to generate an accurate and comprehensive map of the interface between signaling proteins (including, among others, protein kinases, phosphatases, acetyltransferases, ubiquitin conjugating enzymes, and receptors) and TFs. This work will replicate the equivalent map generated for human B cells and will provide important clues about signaling pathway conservation in distinct cellular contexts (100; herein incorporated by reference in its entirety). Appropriate metrics will be used to assess the quality of the results, including overlap of predicted interactions with protein-protein interaction databases and NetworKIN algorithm inferences (50, 100; each herein incorporated by reference in its entirety). Additional opportunistic assays will be used to validate interactions of specific biological value. The analysis will be used to:

Identify Modulators of MRM TF Activity

Upstream modulators of MRM TFs, including Stat3 and C/EBP will be inferred. Modulators that silence the MGES when inhibited provide candidate therapeutic targets and will be experimentally followed up in EXAMPLE 5. Conversely, modulators that activate the MGES genes when either inhibited or activated will provide candidate hypotheses for focal gene loss or amplification in tumors, which will be searched from the TCGA-derived tumor Gene Copy Number platforms.

Identify Candidate Post-Translational Master Regulators of the Mesenchymal signature of GBM

As discussed in Ref. 100 (herein incorporated by reference in its entirety) MINDy can be used to associate a regulon* to each non-TF modulator protein. This is an extension of the classical TF-regulon concept to protein that directly or indirectly regulate one or more TFs. A regulon* represents the set of TF-targets indirectly regulated by a protein via the TF(s) it modulates (the modulon). Ref. 100 (herein incorporated by reference in its entirety) shows and biochemically validates that MINDy identified regulons* can be effectively used to identify the signaling proteins targeted by an shRNA silencing assay from GEP differential expression before and after silencing. This effectively validates the ability to infer post-translationally acting MRs. Specifically, MINDy will first be used to infer a regulon* for each analyzed signaling protein and then the MR approach will be applied to determine significance of regulon* overlap with MGES genes. Signaling proteins whose regulon* is significantly enriched in MGES genes will be (a) considered candidate post-translational MRs, (b) experimentally validated using siRNA assays, and (c) tested for genetic and epigenetic alterations.

Extension of the Enrichment Analysis

FET p-values are strongly dependent on datasets size. Additional approaches will be explored, such as the GSEA (92; herein incorporated by reference in its entirety), as discussed in Ref. 49 (herein incorporated by reference in its entirety). This requires a list L I of available genes ranked by their differential expression between two phenotypes and a list L2 of genes of interest (i.e. the MGES). Whether L2 is enriched in genes that are most up- or down-regulated in L1 will be tested. Since GSEA corrects for gene set size, this will be less sensitive to regulon/modulon size.

MINDy Extensions

MINDy is the first algorithm able to identify post-translational modulators of TF activity from gene expression profile data. However, it has several limitations that can prevent specific modulators from being identified. MINDy uses an extremely conservative, Bonferroni-corrected significance threshold for the CMI analysis because of the large number of tested modulator-TF-target triplets. Thus, some significant triplets can be missed causing two problems: (a) increased false negatives among TF-targets and (b) increased false negatives among inferred modulators. Less conservative threshold for triplet selection will be used and compute a null hypothesis on the minimum number of significant triplets with same TF and modulator, necessary to declare the modulator-TF interaction statistically significant. This is similar to the notion of statistical enrichment in GSEA where a set of genes, each one with modest p-value (i.e. not statistically significant on a single-gene basis), produces significant p-value for the gene set. In the preliminary results, this approach was used to compute Stat3 and C/EBPβ modulators from 236 ATLAS/TCGA GEPs. Specifically, a threshold of p<0.05, not Bonferroni-corrected, was used to select significant modulator-TF-target triplets. The probability p(n) of observing n significant triplets with the same TF (e.g. Stat3) and modulator was computed. The null hypothesis model was generated by sample-shuffling based CMI analysis. As discussed, this was highly effective in discovering known modulators of Stat3. Additionally, modulators discovered by regular MINDy rank high among the larger set of modulators inferred by this more sensitive analysis (p<1E-4). While the new approach infers many more modulators and modulator-dependent targets, and can have far fewer false negatives, the p-value computed by sample-shuffling can be less conservative. The plan is to correct this problem by exploring a variety of approaches to improve the null-hypothesis generation, such as fitting distribution mixtures, an approach shown to be highly successful in the study of ChIP-Chip data (57; herein incorporated by reference in its entirety). That the new analysis reduces false negatives without substantially increasing false positives will also be validated. Additionally, exploration of additional multivariate metrics such as the information theoretic concept of synergy S[TF; t; M]=1[TF; t; M]−1[TF; t]−1[TF; M] is planned. By replacing the CMI with synergy one can remove the limitation that only modulators that are statistically independent of the TF are inferred by MINDy. Since modulators and TFs can be part of regulatory loops that affect their expression in coordinated fashion, this can also lead to discovery of additional modulators.

Experimental Determination of the Combinatorial Mode of Action of the Mesenchymal TFs in Human Glioma.

Yeast assays have shown that deletion of a TF affects only a relatively modest subset of targets and fails to dramatically affect cell physiology (24; herein incorporated by reference in its entirety). Without being bound by theory, combinatorial regulation by multiple TFs can be more specific and effective in activating and suppressing specific genetic programs in the cell. Coherent FF loops, where two TFs share the same targets and one regulates the other, are well-investigated models to implement such redundant regulation logic. Several studies showed that coherent FF loops with an AND logic reduce transient noise in transcriptional regulation programs, since their targets are effectively regulated only through persistent signals. However, OR logic feed-forward loops can also compensate for the loss of a single TF. Thus, it is important to address the role of the regulatory motifs within the inferred MRM to discriminate their ability to filter transient noise from that of providing transcriptional redundancy. Specifically, one behavior is associated with synergistic control (i.e. both TFs are required for target regulation) while the other is associated with additive (i.e. compensatory) control (one TF compensates for the other but the effect is stronger in combination). Discriminating between these two “regulatory logics” is important to understand disease etiology and determine appropriate therapeutic targets.

In EXAMPLE 2, it was shown that at least 80% of the regulatory regions of the genes predicted as first neighbor of the mesenchymal TFs by the ARACNe network are physically bound by the corresponding TFs (FIG. 3). However, individual binding assays fail to characterize the complexity of the regulatory region upstream of a gene providing only a lower-bound on the actual TF binding activity. Thus, the full scope of the direct regulatory activity of the mesenchymal TFs for the mesenchymal subnetwork can only emerge from genome-wide ChIP assays (ChIP-on-Chip). Since preliminary data indicate that Stat3 and C/EBPβ, are both necessary and sufficient to induce the mesenchymal signature genes, one can obtain high-resolution maps of their genome-wide chromatin interactions by ChIP-on-Chip analysis.

The ChIP-on-chip Significance Analysis (CSA), a method for ChIP-Chip data analysis, was recently described, which significantly improves specificity and sensitivity (57; herein incorporated by reference in its entirety). For this reason, CSA is suited to identify regulatory program overlap of multiple TFs. CSA was used to demonstrate that 93% of NOTCH1 bound promoter also bound MYC (57; herein incorporated by reference in its entirety). This cannot be possible with methods yielding higher false negative rates. This analysis will provide a set of targets bound by both TFs, which can be interrogated in functional assays for synergistic vs. additive regulatory control. Individual TF-DNA complexes will be immunoprecipitated from the human “mesenchymal” glioma cell line SNB75 (FIG. 2) and hybridize global tiled arrays (Agilent Technologies) covering promoter regions of annotated human genes (approx. 17,000 genes). DNA microarrays contain 60-mer oligonucleotide probes covering the region from −8 kb to +2 kb relative to the transcription start sites for annotated human genes. This analysis will allow determination of the full set of Stat3 and C/EBPβ-occupied genes in human glioma cells, as well as their overlap. Consequently, one will be able to determine whether, as predicted by original computational analysis, the promoters of the 136 mesenchymal signature genes are enriched among the Stat3-C/EBPβ-occupied promoters in the genome. Although some TFs regulate genes from distances greater than 8 kb, 98% of known binding sites for human TFs occur within 8 kb of target genes. For these assays state-of-the-art ChIP-on-Chip protocols and DNA microarray technology that are known to minimize false positive rates will be used (12, 70; each herein incorporated by reference in its entirety). Most of the initial ChIP-on-Chip experiments used genomic arrays comprised of PCR products that only allowed crude mapping of binding sites and often resulted in lower quality results. The more recent experimental platforms for these assays use oligonucleotide tiling arrays that allow far higher resolution mapping of the binding regions by covering the region where an interaction can be detected with multiple independent probes, thus reducing both false positives and false negatives.

Biochemical and Computational Analysis

ChIP and ChIP-on-Chip experiments will be done according to the protocols recently described (31, 41, 57, 70; each herein incorporated by reference in its entirety). Bound genomic regions will be identified using CSA, which has been shown to produce a 10-fold increase in biochemically validated bound sites (57; each herein incorporated by reference in its entirety). For example, a global, genome-wide analysis can exhaustively determine the full set of Stat3 and C/EBPβ-bound promoters and establish whether the promoters of the 136 mesenchymal signature genes are enriched among the Stat3-C/EBPβ-occupied promoters. Therefore, the ChIP-on-Chip experiments will be expanded to a global, genome-wide scale. Chromatin immunoprecipitation products will be hybridized onto tiled arrays (commercially available from Agilent Technologies) covering promoter regions of annotated human genes (approx. 17,000 genes). A method that significantly improves ChIP-Chip analysis (ChIP-Chip Significance Analysis, CSA) will be carried out (57; each herein incorporated by reference in its entirety). CSA was used to show the almost perfect overlap between promoters binding NOTCH1 and MYC (93% of NOTCH1 binding promoters also bind MYC). Because of its very low false negative and false positive rate, CSA is uniquely suited to show the overlap between Stat3- and C/EBPβ-bound promoters.

Briefly, this approach generates a much more realistic null hypothesis for ChIP-Chip data by modeling the IP/WCE ratio (IP=Immunoprecipitated protein channel, WCE=whole cell extract channel) for unbound sites. This is done by fitting a non-parametric probability density to the left tail of the IP/WCE distribution, which is essentially not-affected by DNA binding events, and using it to extrapolate the right tail of the distribution to obtain a realistic p-value for rejecting the null-hypothesis. The approach has led to the identification of almost perfectly overlapping transcriptional programs, such as those of the Notch1 and MYC TFs in T cells, overlapping on 1,668 of the 1,804 genes bound by Notch1 (92.5%, p-value=3.6×10⁻¹²). As a result, it will be useful to determine the true extent and identity of the Stat3 and C/EBP target overlap. It will also provide high-accuracy bound sites that can be interrogated using a variety of DNA binding site analysis tools, such as DME (84-87; each herein incorporated by reference in its entirety) to identify known TFs whose DNA-binding profiles matches are enriched in the bound vs. unbound fragment as well as to discover new DNA-binding profiles de novo. Both approaches will be used to fully characterize the cis-regulatory modules that support the combinatorial regulation of the targets by multiple TFs and to infer synergistic TF interactions. The Promoclust tool (88; herein incorporated by reference in its entirety), which uses permutation pattern discovery across orthologous regulatory sequences in related organisms, will be performed to identify conserved cis-regulatory motifs comprising multiple DNA binding sites. This method will be applied to the analysis of the MGES genes to identify specific regions where TFs, including Stat3 and C/EBPβ can interact. This will identify the sites mediating possible synergistic regulation by TF-complexes. Validation of promoter occupancy will be performed by quantitative PCR analysis of IP and their corresponding WCE as described in recent publications (57, 70; each herein incorporated by reference in its entirety).

Combinatorial Regulation

As previously shown, ARACNe inferred targets of the MRM TFs are highly overlapping (see Table I). Without being bound by theory, some of the MRM TFs can form transcriptional complexes supporting a combinatorial logic. To test this possibility immunoprecipitation assays for each individual TF followed by Western blot for any of the other candidate synergistic TFs identified by ARACNe or by the cis-regulatory module analysis will be performed. For most of the currently identified MRM TFs (Stat3, C/EBPβ, bHLHB2, and FosL2), antibodies are available and were validated in the ChIP assays shown in FIG. 3. For additional MRM TFs, including those emerging from the additional ARACNe and cis-regulatory module analysis, appropriate antibodies will be identified, when available, and identical testing will be performed. Positive results will be further investigated by testing whether any of the candidate interaction occurs directly through in vitro experiments in which one of the two TF, expressed as a GST-fusion protein, will be interrogated for its ability to capture the candidate interacting factors that had been synthesized from a rabbit reticulocyte lysate. Without being bound by theory, an interaction between an activator TF and a repressor TF can function to restrain the activity of the activator TF bound to the DNA regulatory region of the mesenchymal promoters. Overexpression and silencing experiments of the genes coding for the TFs will interrogate the functional consequences of TF-TF interactions for the expression of selected mesenchymal genes and/or the entire MGES.

Stat3 and C/EBPβ as Targets to Impair Brain Tumor Formation.

It has been shown that constitutive expression of Stat3 and C/EBPβ induces the MGES in NSCs and confers them the ability to develop tumors (FIGS. 5-6). These findings establish that Stat3 and C/EBPβ are sufficient to promote mesenchymal transformation of NSCs. However, the ultimate goal is to exploit the computationally inferred MRs as invaluable targets for therapeutic intervention in malignant glioma. Without being bound by theory, functional inactivation of the drivers of MGES in glioma collapse not only the gene expression signature but also the phenotypic hallmarks endowed by the signature, namely glioma tumor aggressiveness. This will be tested in GBM-BTSCs, a cellular system modeling human GBM in vitro and in vivo. Thus, Stat3 and C/EBPβ will be depleted using a tetracycline regulatable lentiviral system (94; herein incorporated by reference in its entirety) and the functional consequences of loss of Stat3 and C/EBPβ in GBM-BTSCs will be explored. Two assays—one determining the percentage of clone-forming neural precursors (clonogenic index) and the second assessing the expansion of neural stem cell pool by growth kinetics analysis—will be used to determine the consequences of Stat3 and C/EBPβ silencing on self renewal of GBM-BTSCs.

Next, the expression of CD133, a marker enriched in normal and tumor stem cells of the nervous system will be measured. Without being bound by theory, silencing of Stat3 and C/EBPβ will limit stem cell behavior of GBM-BTSCs. Possible outcomes of silencing of Stat3 and C/EBPβ in GBM-BTSCs are growth arrest associated with differentiation along one or multiple neural lineages or apoptosis. Therefore, the expression of specific markers for the neuronal, astroglial and oligodendroglial lineage will be determined, proliferation rate will be measured by immunostaining for BrdU and apoptotic response will be tested by Tunel assay and Annexin V immunostaining. In order to obtain statistically relevant results in vitro experiments will be conducted in at least five independent GBM-BTSCs lines. The effects of Stat3 and C/EBPβ silencing on the tumor initiating capacity of GBM-BTSCs will be tested in vivo by the transplantation of GBM-BTSCs into the mouse brain. Transplantation of GBM-BTSCs into the brain of immunodeficient mice generates highly aggressive tumors displaying each of the phenotypic hallmarks of human GBM (proliferation, anaplasia, tumor angiogenesis, necrosis, formation of pseudopalisades). Consistent with the notion that lentiviruses efficiently transduce neural precursors (94; herein incorporated by reference in its entirety), infection of more than 90% of GBM-BTSCs cultures is routinely obtained. For silencing experiments, a small hairpin RNA expression cassette targeting endogenous Stat3 and C/EBPβ (H1-Stat3 shRNA or H1-C/EBPβ shRNA) is inserted downstream of the tetO sequence. The advantages of this design in a single vector is tight tet-dependent regulation of either the transgene or the shRNA, a fast on to off or off to on kinetics and high levels of drug responsiveness (94; herein incorporated by reference in its entirety). Moreover, the conditional knockdown of the selected endogenous gene is mirrored by the expression of GFP by the transduced cells, thus facilitating monitoring.

Transduction of GBM-BTSCs with lentiviruses will be performed following protocols established in the past for lentivirus-mediated transduction of NSCs and routinely used in our laboratory (11, 16; each herein incorporated by reference in its entirety). The key aspect of GBM-BTSCs cultures is the ability of such cells to maintain their stem cell state when grown as neurospheres in serum-free medium containing EGF and bFGF. To initiate exit from the stem cell state and promote differentiation, single cell suspensions will be cultured in the absence of serum and growth factors and allowed to adhere onto Matrigel-coated glass coverslips. To analyze differentiation, cells will be fixed in 4% paraformaldeyde and processed for immunofluorescence of neural antigens. To evaluate tumorigenicity in the brain, lentivirally transduced BTSC will be orthotopically transplanted following washing and resuspension in PBS at the concentration of 10⁶ cells per ml (injection volume: 10 μl).

To activate the expression of Stat3 and C/EBPβ shRNA, mice will be treated by oral doxicyclin. Ten mice per group will be injected and survival analysis will be established by Kaplan-Meyer Longrank test. Without being bound by theory, inactivation of mesenchymal TFs impairs tumor formation and/or decreases migration and angiogenic capability. Similar experiments will be performed to ask whether enforced expression of ZNF238 synergizes with silencing of positive TFs to trigger the collapse of the MGES and suppresses the biological attributes of glioma aggressiveness that are linked to this signature.

### Example 4

### To Elucidate the Mechanism of ZNF238 Silencing in High-Grade Glioma and Test the Role of ZNF238 Gene Loss in Gliomagenesis in the Mouse

Somatic mutations affecting large TF hubs, controlling a large number of targets, have been shown to be associated with cancer (26; herein incorporated by reference in its entirety). Loss of multiple components and dysregulated expression and/or activity of key oncogenes and tumor suppressor genes occur in most forms of cancer. ZNF238 is the only large TF hub that emerged from the ARACNe analysis of GBM microarray collection as a candidate repressor of the MGES. We found that ZNF238 mRNA is markedly expressed in normal brain but undetectable in GBM (FIG. 2). Similar patterns of expression of ZNF238 mRNA from an independent set of normal brain vs. GBM samples available from the Oncomine database were detected (FIG. 9). Furthermore, ZNF238 can play important roles for differentiation of neural cells in the brain (8). ZNF238 codes for a 522-amino acid protein (also called RP58) that contains a N-terminal POZ domain displaying homology with the POZ domain of Bcl-6 and four sets of Kruppel-type C2H2 zinc fingers. It associates with condensed chromatin where it recruits the Dnmt3a DNA methyltransferase and is thought to function as a DNA-binding protein with transcriptional repression activity (2, 23; each herein incorporated by reference in its entirety).

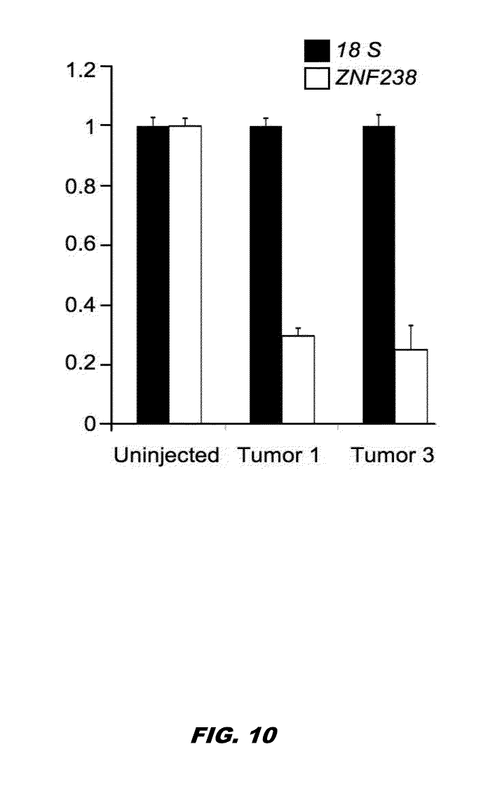

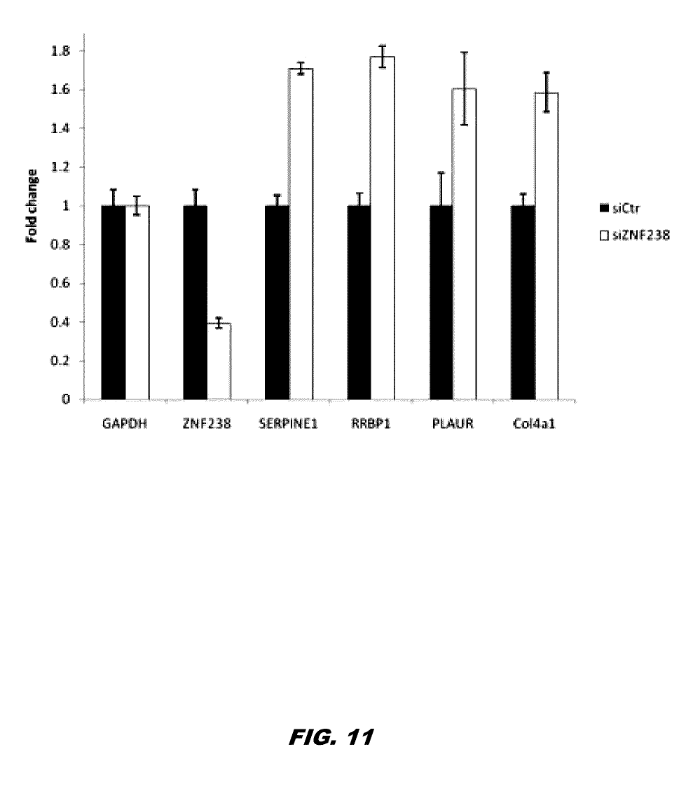

Given the high degree of connectivity in the ARACNe inferred network between ZNF238 and the MGES targets and the significant target overlap between ZNF238 and the positively acting mesenchymal TFs, loss of ZNF238 expression and/or activity is essential to release the normal constrains imposed on the regulatory regions of the MGES genes. Without being bound by theory, loss of ZNF238 in GBM compared to normal brain indicates that loss of ZNF238 is a necessary step in tumor progression. However, the computational and expression data cannot discriminate whether loss of ZNF238 is sufficient or concurrent overexpression of Stat3 and C/EBPβ is also needed to initiate glial tumorigenesis along the mesenchymal phenotype. To test this, the expression of ZNF238 between tumors derived from Stat3-C/EBPβ-expressing NSCs and the same cells cultured in vitro by qRT-PCR as been compared. Interestingly, ZNF238 was markedly down-regulated in the tumor cells in vivo (FIG. 10). This finding raises the intriguing possibility that cells expressing Stat3 and C/EBPβ require ablation of ZNF238 before they emerge into tumors. Furthermore, siRNA-mediated knockdown of ZNF238 in NSCs expressing Stat3 and C/EBPβ led to significant up-regulation of mesenchymal signature genes, thus providing further validation to our finding that ZNF238 is a powerful repressor of the MGES (FIG. 11). Interestingly, the gene encoding ZNF238 maps to chromosome 1q44, a region that is sporadically deleted in human brain tumors (8; herein incorporated by reference in its entirety).

In summary, the ZNF238 gene in NSCs expressing Stat3 and C/EBPβ has been knocked down, and decrease of ZNF238 derepresses the expression of selected mesenchymal signature genes has been shown (Serpinel, PLAUR, Col4A1, see FIG. 11). These findings validate that ZNF238 operates as repressor of mesenchymal signature genes. To further validate that ZNF238 operates as a new tumor suppressor gene in brain tumors, it is shown that: i) ZNF238 is markedly down-regulated in the tumors derived from Stat3-C/EBPβ expressing NSCs (FIG. 10); ii) From the analysis of an independent set of glioblastoma multiforme samples from the Oncomine database for the expression of ZNF238, it was discovered that these human tumors display a significantly reduced expression of ZNF238, when compared with the expression of ZNF238 in normal brain (FIG. 9). Taken together, the new data functionally validate the notion that ZNF238 is a transcriptional repressor of mesenchymal signature genes and strengthen the rationale for the generation of the conditional knockout mouse of ZNF238 in the neural tissue. The systems described herein determine whether ZNF238 is a true tumor suppressor gene for neural tumors and whether it functions to repress the expression of the mesenchymal signature in vivo.

In this example, whether ZNF238 is required to restrain the activity of the MGES in the brain will be examined and whether loss of ZNF238 is a tumor-initiating event in neural cells will be asked. The mechanism(s) of ZNF238 loss in primary glial tumors will be identified through an integrated search of genetic and epigenetic alterations. The specific requirement for ZNF238 in the suppression of malignant transformation will be examined by ablating ZNF238 in the mouse brain. Once generated, ZNF238 mutant mice will be used to ask whether loss of ZNF238 is gliomagenic per se or requires collaborating lesions and evaluate whether concurrent overexpression of ZNF238 target genes contributes to tumor formation. Specifically, whether loss of ZNF238 expression leads to overexpression of the other TFs in the MRM, whether the opposite is true, or whether the two events are independent and both required for oncogenesis will be determined. Finally, GEPs of genetically distinct tumors and cross-species comparisons will be assembled to identify the genetic components necessary to reconstruct the human GBM mesenchymal signature in the mouse. Final outcome of the study will be to establish brain tumor models in which we will test the vulnerability to multi-target intervention strategies. As for Stat3 and C/EBPβ, the HGCM, HGEP1, and HGEP2 dataset will be used to create a repertoire of ZNF238 co-factors and upstream regulators, using the same methodology discussed in EXAMPLES 2-3.

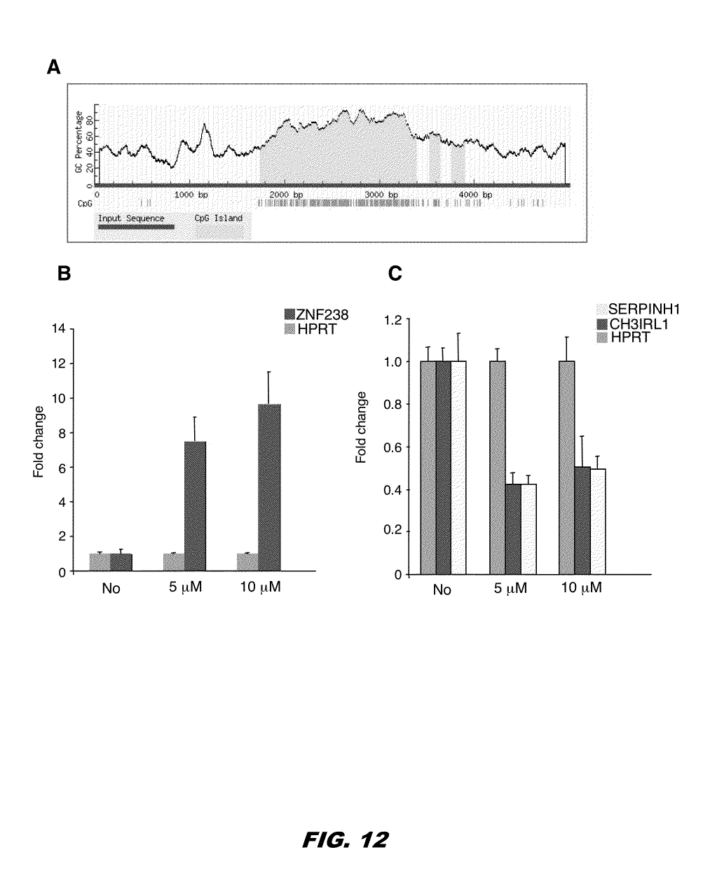

ZNF238 as a tumor suppressor gene in high-grade glioma. Different genetic and/or epigenetic mechanisms can operate, alone or in combination, to silence ZNF238 gene expression in malignant glioma. First and foremost, the ZNF238 gene can be targeted by direct genetic alterations (deletion, recombination such as internal duplication or translocation and mutation). These alterations can specifically target the ZNF238 gene (e.g. point mutations) or be broad and involve also adjacent loci. Furthermore, they can cooperate with other epigenetic alterations to effectively silence the two ZNF238 alleles. A prior analysis of the genetic platforms available from the ATLAS TCGA network did not identify major rearrangements in the ZNF238 locus. However, focal alterations of the ZNF238 gene can only be excluded after complete resequencing of the corresponding genetic locus in a significant number of brain tumors samples. Furthermore, it is recognized that, in the absence of changes in the coding region, genetic alterations in the ZNF238 regulatory region (promoter) can knock out a crucial enhancer activity for ZNF238 mRNA expression in the nervous system. Therefore, beside the analysis of the ZNF238 coding region, the analysis will have to include the full ZNF238 promoter. The relevance of ZNF238 promoter targeting in brain tumors is underscored by the preliminary finding that the ZNF238 promoter is aberrantly methylated in glioma cells (FIG. 12).

Promoter methylation is a frequent mechanism for inactivation of tumor suppressor genes in human tumors and it will be explored in the next paragraph. Here, whether the ZNF238 promoter and/or its coding sequence are targets for broad or focal alterations in malignant brain tumors by double strand sequencing of tumor DNA is considered. The availability of 200 frozen GBM specimens harvested from anonymous donors and stored in the brain tumor bank of the Columbia Cancer Center Tissue Bank will be taken advantage of. The ZNF238 gene in the 18 human glioma cell lines available in the laboratory will be sequenced. The entire ZNF238 promoter (4,000 by upstream of the transcription start site) and coding region from genomic DNA derived from 200 GBM specimens will be sequenced. The primer pairs required for successful PCR amplification followed by direct double-strand sequencing coverage have been validated. Functional experiments will validate the significance of any ZNF238 mutation identified in the sequencing screen. The type of genetic mutation that will be detected in brain tumors can immediately direct one towards the functional consequences produced by that genetic event. However, subtle mutations in putative TF-binding sites in the ZNF238 promoter (e.g. point mutations) are detected, experiments will be designed to establish the consequences of the mutation on ZNF238 promoter activity by using luciferase-reporter assays. The assays will be conducted by preparing plasmid constructs in which the wild type ZNF238 promoter and the corresponding mutant(s) will be placed in front of a luciferase reporter gene. This system allows accurate quantitation of promoter activity and is ideally suited to identify the partial reduction of ZNF238 promoter activity that can be associated with certain mutations in TF-binding sites. Execution and evaluation of promoter-luciferase assays have been shown (31, 41; each herein incorporated by reference in its entirety). An alternative/complementary mechanism to the direct genetic inactivation of ZNF238 can include genetic/epigenetic targeting of upstream regulators of ZNF238. ARACNe can be used to infer TFs that are candidate upstream regulators of ZNF238, as described in EXAMPLE 3. A similar experimental plan will be implemented to search for alterations in the genes coding for these modulators. The availability of the ATLAS TCGA genetic platforms will be instrumental to identify/exclude major rearrangements.

Analysis of Promoter Methylation of ZNF238.

Computational and expression predictions converged towards the identification of a highly vulnerable structure of the regulatory region controlling ZNF238 expression. The ZNF238 promoter/enhancer is unusually rich in evolutionarily conserved CpG islands (FIG. 12A), which are targeted by DNA methyltransferases leading to gene expression silencing. Methylation of regulatory DNA regions is a common mechanism in human cancer and is implicated in the constitutive silencing of tumor suppressor genes in malignant glioma (109; herein incorporated by reference in its entirety). Thus, whether promoter methylation induces silencing of ZNF238 was considered. Pharmacological inhibition of methylation with an inhibitor of DNA methyltransferases (5-Azacytidine) elevated the expression of ZNF238 mRNA in the T98G glioma cell line (FIG. 12B) and repressed the expression of SerpineH1 and CH3IRL1 (FIG. 12C), two mesenchymal genes predicted as ZNF238 targets by ARACNe (FIG. 1). These results indicate that the aberrant methylation of the ZNF238 promoter can account for silencing of ZNF238 expression in primary GBM.

The extent by which the ZNF238 promoter is aberrantly methylated in the collection of 200 human GBM will be determined. Methylation status of the promoter regions of ZNF238 will be analyzed by matrix-assisted laser desorption ionization time-of-flight mass spectrometry (MALDI-TOF MS) of PCR-amplified, bisulfate-modified high grade glioma DNA, as previously described (Sequenom, San Diego, Calif.) (19, 89; each herein incorporated by reference in its entirety). This method allows semiquantitative, high-throughput analysis of methylation status of multiple CpG units in each amplicon generated by base specific cleavage. The PCR product is cleaved U specifically. A methylated template carries a conserved cytosine, and, hence, the reverse transcript of the PCR product contains CG sequences. In an unmethylated template, the cytosine is converted to uracil. The reverse transcript of the PCR product therefore contains adenosines in the respective positions. The sequence changes from G to A yield 16-Da mass shifts. The spectrum can be analyzed for the presence/absence of mass signals to determine which CpGs in the template sequence are methylated, and the ratio of the peak areas of corresponding mass signals can be used to estimate the relative methylation. This assay enables the analysis of mixtures without cloning the PCR products.

The ZNF238 gene contains a large CpG island of approximately 2 kB that lies upstream of the coding region. Four independent amplicons that cover the entire region (#1, −3576 to −2894; #2, −2878 to −1643; #3, −1619 to −1416; #4, −1197 to −1090) will be analyzed. Methylation data will be viewed in GeneMaths XT v 1.5 (Applied Maths, Austin, Tex.). Similar approaches will be used to investigate co-factors and upstream regulators of ZNF238 that can emerge from the ARACNe analysis. Studies of the mechanisms of inactivation of tumor suppressor genes in primary tumors have been described (15, 32, 69; each herein incorporated by reference in its entirety) and, depending on the outcomes of the initial experiments, specific experimental strategies will be designed to validate the significance of genetic and/or epigenetic inactivation of ZNF238 in GBM and of any additional negative regulator of the mesenchymal signature genes emerging from the analysis.

Analysis of the Functional Effects of ZNF238 Expression in Glioma Cells.

A fundamental assay to test whether a gene has tumor suppressor function is its ability to inhibit tumor growth when re-introduced in cancer cells. Thus, whether ZNF238 fits this criteria will be evaluated by re-expressing the ZNF238 gene in the human glioma cell lines that lack endogenous expression of ZNF238. Through the use of a tetracycline-inducible system, the impact of ZNF238 expression for the MGES will be evaluated and the following functional experiments will be performed: i. Evaluate the effect of ectopic ZNF238 expression on cell proliferation in the glioma cell lines SNB75, T98G and SNB19. Like primary GBM, none of the three cell lines express detectable amounts of ZNF238 mRNA (FIG. 2). The effects of ZNF238 expression for proliferation will be tested by colony assays, cell counting, BrdU incorporation and FACS analyses; ii. Ask whether reconstitution of ZNF238 expression in glioma cells perturbs the ability to migrate and invade through the extracellular matrix using the in vitro and in vivo assays shown in FIGS. 5-6. These are the major phenotypic features of the MGES and similar experiments will also be done in the context of concurrent silencing of one or more of the positively connected “mesenchymal TFs.” Any additional key tumor suppressor gene candidate, emerging from the computational analysis, will be tested using similar approaches. In case a hierarchical control structure emerges from the analysis, one can start by validating the genes that are most upstream in the regulatory logic.

Generation of Mice Carrying a Conditional Mutant Allele of ZNF238.

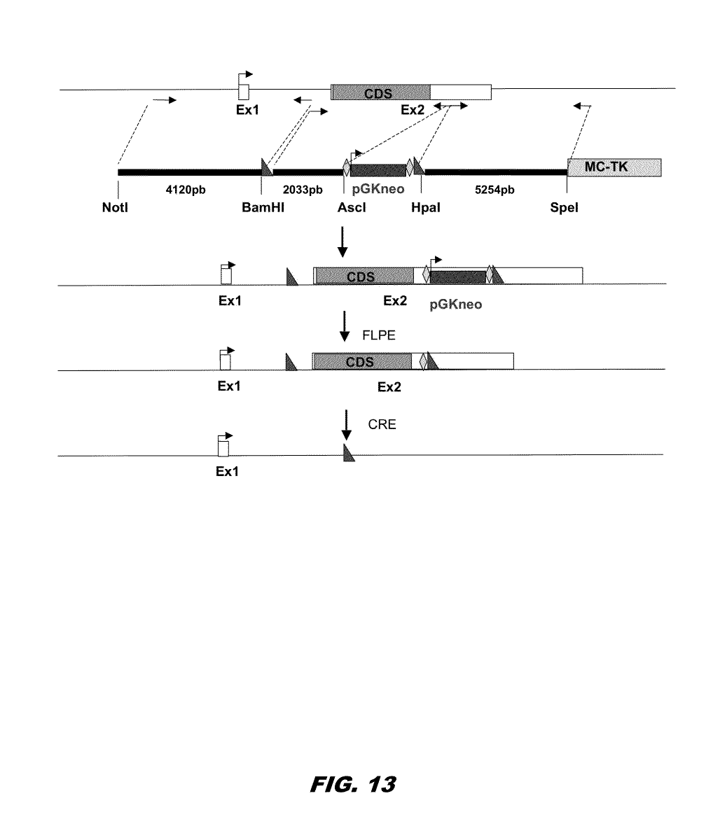

Although in vitro experiments can provide valuable insights, the validation of ZNF238 as repressor of the MGES and glioma tumor suppressor gene comes from the genetic analysis of ZNF238 function in vivo. Therefore, one can develop a ZNF238 allele (ZNF238Flox) that contains LoxP sites flanking exons 1 of the mouse ZNF238 gene (FIG. 13). Exon 1, which contains the entire ZNF238 coding sequence, is deleted after expression of Cre recombinase to generate a ZNF238 null allele. Once the appropriate constructs have been generated and sequence verified, the final targeting vector is electroporated into mouse embryonic stem cells (ES) and, after G418 selection, ES colonies will be screened for recombination events by Southern blotting and PCR. Appropriate clones will be used to generate chimeric mice by microinjection into C57BL/6 blastocysts. F1 animals will be screened for germ line transmission of the mutant ZNF238 allele by tail-DNA genotyping. This will involve direct sequence of PCR products as well as southern blotting to demonstrate ablation of ZNF238. The primary focus will be to establish the function of ZNF238 in the nervous system. To achieve specific inactivation of the ZNF238 in the nervous system, ZNF238Flox mice will be crossed with the GFAP-Cre deleter strains to generate GFAP-ZNF238Flox. GFAP-Cre mouse strains are already available in our facility. Conditional knockout mouse models have recently been generated for three different genes (Id2, Id1 and Huwe1) and one is fully equipped to generate this new genetically modified mouse. Other mouse tumor models based on Cre-mediated recombination have been generated and tested (51, 52; each herein incorporated by reference in its entirety).

Analysis of GFAP-ZNF238 Conditional Mutant Mice to Address the Role of ZNF238 Loss in Tumor Development in the Brain.

The GFAP promoter is active in most embryonic radial glial cells that exhibit neural progenitor cells properties and mature astrocytes (53, 54, 67, 112; each herein incorporated by reference in its entirety). Early onset of the activity of the GFAP promoter in progenitor cells leads to Cre-mediated recombination in early neural cells as well as their progeny, including a large array of neural stem/progenitor cells in the sub-ventricular zone of the adult mouse as well as in mature neurons, astrocytes oligodendrocytes and cerebellar granule neurons (53, 54, 59, 62, 97, 112; each herein incorporated by reference in its entirety). One can compare the tumor initiating potential of ZNF238 loss with or without mutation in tumor suppressor gene NF1. The choice is based on the following data: 1) Individuals afflicted with neurofibromatosis type 1 (NF1) are predisposed to malignant astrocytoma in the brain (80; herein incorporated by reference in its entirety), and 2) Mice carrying NF1 loss in the GFAP-positive compartment in the brain (GFAP-Cre; Nf1 flox/flox) exhibit increased numbers of brain and optic nerve astrocytes, but they do not develop gliomas (5; herein incorporated by reference in its entirety). Therefore, they represent a model system to identify a specific role for loss of ZNF238 in transformation of neural cells. Nf1 flox mice are available through the NCI Mouse Models of Human Cancer Consortium. Additionally, one can consider other candidate oncogenes and tumor suppressor genes emerging from the MGES transcriptional program modeling effort described earlier.

ZNF238Flox mice will be crossed with hemizygous GFAP-cre transgenic mice (38; herein incorporated by reference in its entirety), generating GFAP-ZNF238Flox mice and then bred to appropriate strains to yield GFAP-ZNF238Flox; Nf1Flox/Flox progeny for the analysis. Genotyping of ZNF238 and NF1 alleles will be performed by PCR. Offspring with conditional mutation of ZNF238 will be examined for neural defects. If the ZNF238 mutant mice develop differentiation and/or proliferation abnormalities, one can use gene expression microarray to determine whether such abnormalities are sustained by deregulated activity of the MGES in vivo.

One can determine the kinetics of tumor formation by daily clinical examination and serial pathology. Adult mice will be monitored for development of tumor associated signs and sacrificed appropriately. Tumor tissue will be isolated, fixed for immunostaining and frozen for DNA/RNA/protein analysis. Tumor latency, penetrance and histopathological features will be monitored. Pathological examination will include, H&E for morphology, BrdU for proliferative index, and Tunel for apoptotic rates. Immunohistochemical marker analysis for GFAP, NeuN and Synaptophysin will be used to confirm or rule out glial or neuronal lineage of the tumor, respectively. Further characterization will include Nestin immunohistochemistry to uncover NSCs and early glial progenitors. Whenever possible, cell lines will be derived from tumors for biochemical analysis or explant studies. A key objective of the studies is to perform a transcriptomic microarray analysis of the tumor samples to generate a map of the mesenchymal signature in different biological states. To determine the extent to which mouse cancers express the GBM mesenchymal signature in a manner resembling the human tumors, the genes in the GBM mesenchymal signature will be used to cluster the mouse tumor data set hierarchically.

To determine whether there is hierarchical causal function of the mesenchymal TFs for tumor formation in a ZNF238-null background, the genes coding for each mesenchymal TF will be ectopically expressed either individually or in combination by in vivo electroporation of retroviral vectors. The requirement of these same genes will be tested by stably decreasing their expression in vivo with short-hairpin RNA-mediated interference (RNAi) lentivirus. Lentiviral and retroviral vectors for gene expression or silencing that co-express GFP are routinely used. These vectors will allow one to track infected cells. Tumors will be examined for histology and gene expression profiling. Collectively, results from these experiments will reconstruct in vivo the mode of cooperation of ZNF238 with the mesenchymal TFs for MGES expression and brain tumor formation.

Without being bound by theory, GFAP-ZNF238LoxP mice will develop proliferative alterations in the brain and loss of NF1 accelerates tumor formation and/or increase malignancy. It has been shown that the only proliferating cells in the adult mouse brain are those in the SVZ (18; herein incorporated by reference in its entirety). Therefore, this extremely low background will permit a sensitive survey of the brain for proliferating cells by BrdU incorporation. Further analysis of the regulatory control responsible for differentiating ZNF238 knock-out mice expression from expression in high grade glioma can provide additional insight on key co-factor of this TF required for oncogenesis.

### Example 5

### To Computationally Identify and Biochemically Validate “Druggable” Proteins and Co-Factors that Modulate the Mesenchymal Signature in GBM

Without being bound by theory, MGES genes will be dysregulated by several processes, including epigenetic silencing, gene copy number alterations, regulation by additional TFs missed by the preliminary analysis, and genetic/epigenetic alterations of regulators upstream of the identified regulatory module. For the latter, one can especially focus on modulators upstream of Stat3, C/EBPβ and ZNF238. For instance, to become transcriptionally competent, Stat3 must be converted to its active form by tyrosine kinase-mediated phosphorylation events (21, 34; each herein incorporated by reference in its entirety). Thus, targeting some of the kinases in this pathway can suppress Stat3 phosphorylation, ablating its transcriptional activity.

In this example, one can (a) investigate complementary approaches to identify candidate pharmacological targets and compounds for MGES silencing and (b) validate their ability to reduce the aggressive phenotype of high-grade gliomas. A first more “targeted” approach will investigate specific upstream modulators of Stat3, C/EBP, ZNF238, and other MGES MRs from EXAMPLES 2-4. The second approach will use the High-grade Glioma Connectivity map (HGCM) to investigate druggable proteins as candidate MGES modulators. Druggable proteins will be identified using the Druggable Genome database (30; herein incorporated by reference in its entirety). Candidate targets will first be prioritized and screened in silico and then tested in vitro using siRNA silencing assays. The targets emerging from this analysis will also be tested for synergism to model the combinatorial regulation of the MGES. Finally, one can use several computational, literature-based, and experimental approaches to identify compounds that can target the MGES modulators identified by this analysis and test them in vitro and in vivo for the ability to block glioma cell proliferation and invasion.

Targeted approach. One can start with a collection of (a) MINDy inferred candidate modulators of the MGES regulatory module's TFs (see EXAMPLE 3) and (b) candidate MRs of the MGES genes inferred by the regulon*-based MRA (see EXAMPLE 3). Inferred modulators will be first filtered, using the Druggable Genome database (30; herein incorporated by reference in its entirety), to identify Candidate Pharmacological Targets (CPT) and associated compounds. In the MYC modulator analysis, ˜50% of the 30 highest-confidence MINDy inferred modulators were bona fide MYC modulators in vitro (101, 102; each herein incorporated by reference in its entirety). This is a lower bound, because the untested genes can include additional modulators. One can use the statistics defined in Ref. 101, 102 (each herein incorporated by reference in its entirety) to identify high-confidence candidate modulators of the MGES MRs and appropriate statistics will be developed to infer equally high-confidence candidate MGES MRs using the regulon*-based approach.

Validation will proceed in two steps and will be used to inform the “unbiased” approach described herein. Modulators will be divided in two categories, depending on biological activity. TF activators will include genes that increase the TF's transcriptional activity while antagonists will include genes that repress it. Since most drugs act as substrate inhibitors, only activators of the MGES positive regulators (e.g. Stat3 and C/EBPβ) and antagonists of MGES negative regulators (e.g. ZNF238) will be considered. Similarly, for genes inferred by modulon-analysis, only MGES activators will be considered, such that their chemical inhibition can result in down-regulation of the signature. Based on previous analyses, and without being bound by theory, about 30-50 candidate targets could emerge from this analysis. One can use a two-step screening approach to minimize cost and maximize changes for correct target identification. In the first phase, one can pool siRNAs directed against three sequences to silence each one of the candidate targets and can perform qRT-PCR to validate suppression of the corresponding target mRNA. Samples showing substantial (>70%) reduction in mRNA level will be hybridized to Illumina arrays in duplicates. One can then compute the GSEA enrichment of differentially expressed genes against the MGES to determine the contribution of silencing candidate targets to MGES abrogation. Furthermore, use of two replicates can provide adequate power to test enrichment of a large TF signature, including 50 to several hundred targets. Without being bound by theory, a smaller number of candidate modulators will show significant repression of the MGES. These will be validated using the individual siRNAs in the pool and additional siRNAs, if available, to exclude possible off-target effects. Specifically, one can test that siRNAs that induce silencing of the target modulator will show a consistent repression of the MGES. Finally, one can test the effect of compounds that are reported in the database as active on specific targets emerging from this analysis.

Unbiased Approach.

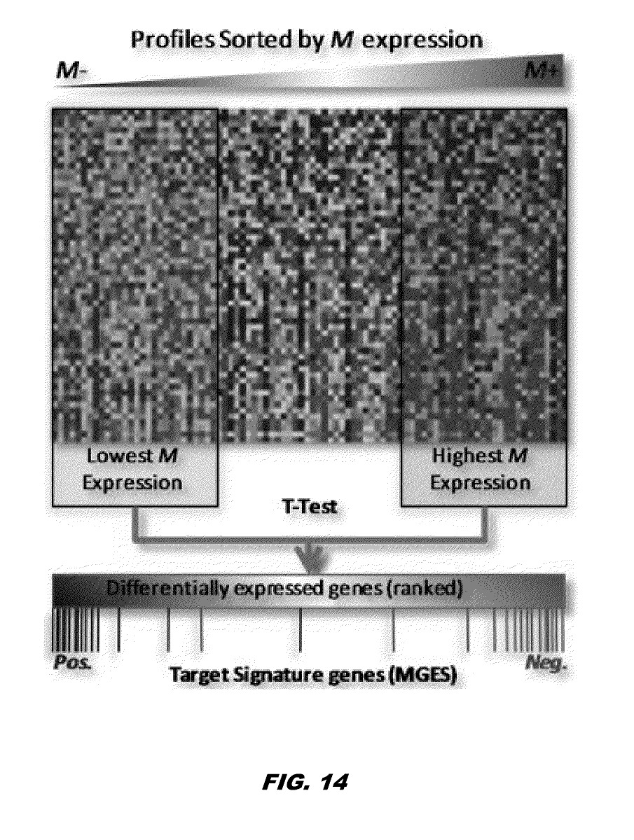

The availability of the HGCM from EXAMPLES 2 and 3 will inform approaches tested in the MCF7 breast cancer cell line (48; herein incorporated by reference in its entirety). A key advantage of this approach is that candidate druggable targets will be tested directly against the MGES, without requiring interaction map inference. Thus, it can provide targets whose connectivity can not have been appropriately reconstructed by ARACNe or MINDy. FIG. 14 illustrates the process for one candidate druggable target gene. This will be repeated exhaustively for every candidate gene.

For example, if g_{DT} is a CPT in the druggable genome database (30; herein incorporated by reference in its entirety), the following steps will determine if g_{DT} is a candidate MGES activator and thus a candidate target for pharmacological inhibition:

Step 1.

One can first rank-sort the profiles in the HGCM according to the expression of gDT. Since perturbation assays were performed on a single cell line, modulation of g_{DT} can be, on average, the dominant effect, i.e., induced by the chemical perturbation rather than by phenotypic assay variability. The first N profiles will thus represent assays where the perturbation induced transcriptional repression of g_{DT}. This can be called the G↓_{DT} set. Conversely, the last N profiles will represent assays where the perturbation induced transcriptional activation of g_{DT}. This second set can be called the G↑_{DT} set.

Step 2.

One can then assemble a list L of genes ranked according to the t-test statistics computed between the G↓_{DT} and G↑_{DT} sets. N can be chosen to be large enough so that g_{DT}-independent processes are averaged out over the N samples, akin to mean field theory approaches in physics, yet small enough so that average expression of g_{DT} is statistically different. This is similar to the corresponding set selection in MINDy (see EXAMPLES 2-3; where we show that choosing N to be about ⅓ of the total profile population produces optimal results). In this case, since true positive (TP) and false positive (FP) modulators biochemically validated will be available, one can select N such that it produces optimal recall and precision. One can compare the analytically and empirically derived values.

Step 3.

One can finally measure the MGES gene enrichment against differentially expressed genes in L, using the GSEA method. This allows one to treat the two sets, G↓_{DT} and G↑_{DT}, as “virtual” g_{DT} perturbations and the list L as the specific signature that results from that perturbation. In FIG. 14, genes that are activated in the MGES are shown as short, blue, vertical lines. Repressed genes are shown as short, red, vertical lines. GSEA analysis will pinpoint g_{DT} selections that will respectively enrich the blue genes among genes that are upregulated in G↑_{DT} and enrich red genes among genes that are downregulated in G↓DT. This approach was used preliminarily to test which druggable genes induce apoptosis in MCF₇ cells using published connectivity map data (40; herein incorporated by reference in its entirety). It was shown that known apoptosis inducing genes, such as the heat shock protein HSP38, were highly enriched among the top modulators inferred by the approach. Furthermore, testing of 8 high-ranking genes not previously associated with apoptosis, using known chemical inhibitors, identified two compounds that induce apoptosis in vitro with IC₅₀ in the high-nanomolar to low micromolar regimens.

Apoptosis as a consequence of MGES Silencing.

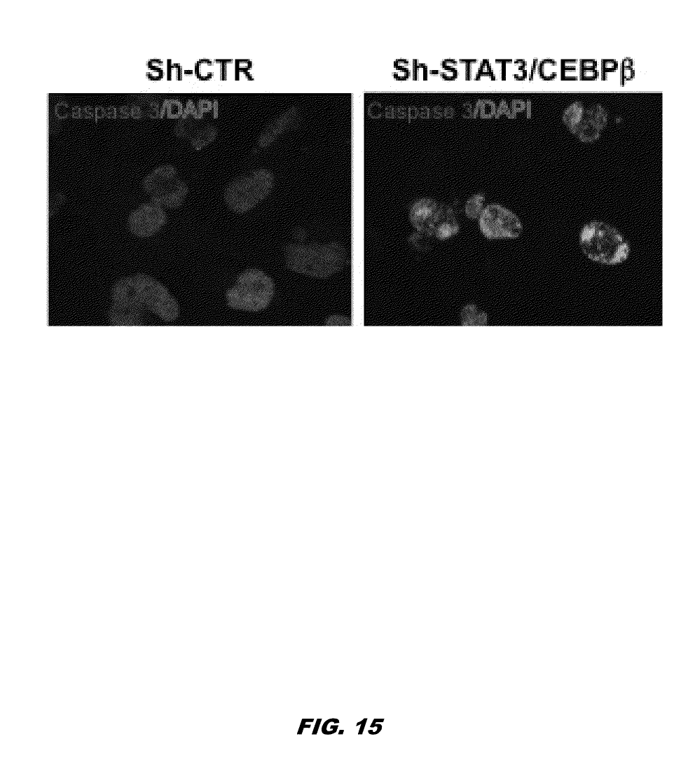

While MGES recapitulates the hallmark of aggressive high-grade glioma, MGES genes are not completely overlapping with the genes that are differentially expressed upon co-silencing of Stat3 and C/EBPβ in GBM-BTSCs. As shown in FIG. 15, such co-silencing produces a markedly apoptotic phenotype, as demonstrated by immunostaining for caspase 3, which can recapitulate tumor oncogene-addiction properties (104; herein incorporated by reference in its entirety). Thus, the analysis of co-silencing of Stat3 and C/EBPβ versus vector transduced controls, will allow one to generate a differential expression signature, distinct from the MGES signature, which will recapitulate the specific effects of knockdown of Stat3 and C/EBPβ in glioma cells. This signature will be used, in addition to the MGES signature, for analyses to identify additional candidate druggable targets that can implement the desired pro-apoptotic phenotype. It will also be used to test the accuracy and properties of the Master Regulator Analysis method (MRA). Without being bound by theory, MRA analysis can predict Stat3 and C/EBP as the MRs of the experimentally induced transformation event. This will allow one to explore alternative metrics for MR ranking purposes and validate the method.

Experimental Validation of MGES Modulators.

Once a repertoire of post-translational modulators of the MRM TFs is identified, they will be first prioritized and validated biochemically in this example and then their biological function will be examined. The repertoire of post-translational modulators will provide a context for the rapid identification of targets of therapeutic value for the suppression of the MGES.

Three distinct but highly integrated approaches will be used:

a) Constitutive and Inducible Expression of Individual Genes

Individual genes that appear to have a critical role in the regulation of MRM TFs will be tested for their ability to influence the regulation of the module through the tetracycline inducible lentiviral system described in EXAMPLE 3. This experimental system has been repeatedly validated with GBM-BTSCs.

b) Inhibition of Individual Gene Expression Via Lentivirus-Mediated shRNA Transduction

The use of shRNAs to inhibit the expression of target genes in transduced cells has been established as the method of choice for ablating the function of individual genes in somatic cells. In EXAMPLE 2, it is shown that shRNA-mediated gene silencing in GBM-BTSCs can be successfully achieved through lentiviral-mediated transduction (see for example the analysis of the effects of silencing Stat3 and C/EBPβ in GBM-BTSCs shown in FIG. 7).

One can use these experimental systems to examine whether i) overexpression of candidate activators of Stat3 and C/EBPβ in NSCs enhances mesenchymal and invasion phenotype in vitro as assayed by immunofluorescence for mesenchymal markers (e.g. SMA, fibronectin, YKL40) and invasion assay and is gliomagenic in vivo following stereotactic injection into the brain; ii) silencing of candidate modulators of Stat3 and C/EBPβ in GBM-BTSCs diminishes the expression of mesenchymal markers, decreases migration and invasion in vitro and inhibits the gliomagenic phenotype in vivo. Similar experiments will be performed to examine the effects of overexpression or silencing of candidate modulators of ZNF238.

c) Pharmacological Inhibition of Specific Targets

An increasing number of pharmacological inhibitors of specific proteins are becoming available. Although some of these inhibitors are not entirely specific for individual gene products, a sizable fraction is used with significant specificity. These pharmacological inhibitors will be very useful experimental tools for the blockage of specific targets and validation of their potential use as therapeutic targets in vivo.

### Example 6

### To Assemble a Human Glioma Interactome (HGi) Including Transcriptional, Signaling, and Complex-Formation Interactions

There are three main types of utilization: 1) one can make the Human Glioma interactome (HGi) available to the research community using the same geWorkbench infrastructure used for the Human B Cell interactome. This will allow the research community to interrogate the HGi to retrieve transcriptional and post-translational interactions for any gene of interest and to identify sub-networks in the HGi that are differentially regulated in various disease sub-phenotypes; 2) one can integrate the HGi with our master regulator analysis tools, also integrated in geWorkbench, to allow the analysis of master regulators of other phenotypes, E.g. low-grade/high-grade vs. normal, rather than high-grade vs. low-grade, which is the subject of this proposal; 3) by extending the IDEA algorithm, one can allow using the HGi as an integrative tool to combine diverse sources of evidence about genetic, epigenetic, and functional alterations to discover sub-networks that are dysregulated within specific sub-phenotypes of interest and to dissect the mechanism of actions of commonly used anti-cancer compounds in these cells.

Recent work has shown that context-specific interactomes can be effectively used as integrative tools to dissect mechanisms of differential regulation/dysregulation in normal and pathologic human phenotypes (49, 55; each herein incorporated by reference in its entirety). In this Example, one can assemble a computationally inferred, biochemically validated interactome for high-grade glioma and use it as a reference anchor to integrate the genetic, epigenetic, and functional data produced by different GBM-related studies. One can integrate data from the ATLAS/TCGA effort, including expression profiles, gene-copy number alterations, promoter hyper and hypo-methylation, and sequence. To assemble the HGi, one can extend the evidence integration methodology described in the attached Ref. 55 (herein incorporated by reference in its entirety). The HGi will include protein-DNA (PD) and protein-protein (PP) interactions specific to glioma cells. The latter include stable (i.e., same-complex) as well as transient (i.e., signaling) interactions. The HGi will be generated by applying a Naïve Bayes Classifier to integrate a large number of experimental and computational evidence.

Appropriate positive and negative “gold-standard” references will be assembled from curated databases, as also described in EXAMPLES 2-5. Evidence sources will include: the four expression profiles defined in EXAMPLES 2 and 3, literature data-mining from Gene Ways (83; herein incorporated by reference in its entirety), TF-binding-motif enrichment, orthologous interactions from model organisms, and reverse engineering algorithms, including ARACNe and MINDy for regulatory and post-translational interaction inference. For each evidence source, a Likelihood Ratio (LR) will be assessed using the positive/negative gold standards. Individual LRs will then be combined into a global LR for each interaction. A threshold corresponding to a posterior probability p≧0.5 will be used to qualify interactions as present or absent. It is important to notice that, given the infrastructure for the assembly of cellular networks implemented by the MAGNet center, one will be able to access a large variety of data sources and algorithms that, otherwise, requires a significant effort to organize and coordinate.

Stable Protein-Protein Interactions.

A Positive Gold Standard (PGS) for PP interactions will be generated using 27,568 human PP interactions from HPRD (76; herein incorporated by reference in its entirety), 4,430 from BIND (4; herein incorporated by reference in its entirety), and 3,522 from IntAct (29; herein incorporated by reference in its entirety). These originate from low-throughput, high-quality assays. The resultant PGS will have 28,554 unique PP interactions between 7,826 gene-products (after homodimer removal). The Negative Gold Standard (NGS) will include gene-pairs for proteins in different cellular compartments, resulting in a large number of gene pairs with low probability of direct physical interaction. Pairs in the NGS that are also included in the PGS will be removed from the NGS. PP interactions will be inferred from the following source: (a) Interactions in the HPRD (76; herein incorporated by reference in its entirety), IntAct (29; herein incorporated by reference in its entirety), BIND (4; herein incorporated by reference in its entirety) and MIPS (63; herein incorporated by reference in its entirety) databases for four eukaryotic organisms (fly, mouse, worm, yeast); (b) human high-throughput screens (82, 91; each herein incorporated by reference in its entirety); (c) Gene Ways literature data mining algorithm (83; herein incorporated by reference in its entirety); (d) Gene Ontology (GO) biological process annotations (3; herein incorporated by reference in its entirety); (e) gene co-expression data from the HGSS, HGES1, and HGES2 expression profiles; and (e) Interpro protein domain annotations (64; herein incorporated by reference in its entirety).

To simplify prior computation, evidence sources will be represented as categorical data (i.e., continuous values will be binned as necessary). Only genes that are both expressed in the glioma expression profiles will be tested for potential interactions. Multiple methods to test for gene expression are being developed, including: (a) standard coefficient of variation analysis (e.g., cv >0.5), (b) methods based on the correlation of multiple probes within Affymetrix probeset for the same gene, and (c) information theoretic approaches based on the ability to measure information with other probesets. These methods will be tested using the PGS and NGS to determine if one is more effective than the others at removing non expressed genes. The prior odds for a PP interaction will be estimated approximately at 1 in 800, based on previous estimates of ˜300,000 PP interactions among 22,000 proteins in a human cell (27, 82; each herein incorporated by reference in its entirety). From this value, any protein pair, after evidence integration, has at least 50% probability of being involved in a PP interaction. PGS PP interactions will also be included in the HGi.

Protein-DNA Interactions.

A PGS for PD interactions will be generated from the TRANSFAC Professional (61; herein incorporated by reference in its entirety), BIND and Myc (MycDB) databases (110; herein incorporated by reference in its entirety). The NGS will include 100,000 random TF-target pairs, excluding pairs in the PGS interaction or in the same biological process in Gene Ontology. A TF-specific prior odds will be used, since the TF-regulon size is approximated by a power-law distribution (7; herein incorporated by reference in its entirety). ARACNe inferences (58; herein incorporated by reference in its entirety) will be used to estimate TF-regulon sizes and to compute the TF-specific prior odds. PD interactions will be inferred from the following evidence sources: (a) mouse interactions from the TRANSFAC Professional and BIND databases; (b) the ARACNe and MINDy algorithms; (c) TF binding site analysis in the promoter of candidate target genes (85; herein incorporated by reference in its entirety); (d) target gene conditional co-expression based on the gene expression profiles defined in EXAMPLES 2 and 3. PGS interactions will be included in the HGi.

Post-Translational Modification.

The MINDy algorithm predicts post-translational modulation events, where a TF and target appear to only have an interaction in the presence or absence of a third modulator gene (M). These 3-way interactions will be split into two distinct pairwise interactions: a PD interaction between the TF and its target and a TF-modulator interaction that can be either a P-TF or a TF-TF interaction, depending on whether the modulator is also a TF. For the interaction types, one can qualify the accuracy and sensitivity of the Interactome using ROC curves based on 5-fold cross validation. Basically, the PGS and NGS will be divided in 5 random subsets of equal size. For each subset, one can train the Naïve Bayes classifier using the remaining four subsets and assess the methods performance using the PGS and NGS subsets that were not used for training the classifier. The MINDy improvements discussed in EXAMPLES 2 and 3 will also be tested to determine the most effective algorithmic approach.

Use of Alternative Classifiers.

Several successful strategies for evidence integration exist and will be considered in alternative to the Naïve Bayes Classifier. These include the use of voting methods (35; herein incorporated by reference in its entirety), Bayesian Networks (36; herein incorporated by reference in its entirety), boosting algorithms (9; herein incorporated by reference in its entirety), and Markov Random Fields (17; herein incorporated by reference in its entirety). The latter is interesting in this context as it allows the integration of functional information on existing network structures.

The HGi as a Framework for Genetic/Epigenetic/Functional Data Integration.

As more and more, largely orthogonal data is amassed to inform analysis of tumorigenesis, a key question is how to integrate this data so that each data modality informs the others. Here, the HGi will be used as an integrative platform for genetic, epigenetic, and functional data related to alterations or dysregulation events in GBM. The simplest level of integration will proceed as in Ref. 55 (herein incorporated by reference in its entirety), by determining whether the topological neighborhood of each gene is enriched in genetic/epigenetic alterations or in interactions that are dysregulated within the malignant phenotype. Each gene or gene interaction will be assigned a score based on the dysregulation events that affect it. For instance, if the promoter of a gene is found to be differentially methylated in cancer samples, then each transcriptional interaction upstream of that gene will be assigned a score. Similarly, if a gene copy number alteration affecting a region that includes N genes is detected, then each gene will be assigned a score. Differential mutual information on each interaction in normal vs. malignant samples will also be used to assign a dysregulation score to each gene-gene interaction (55; herein incorporated by reference in its entirety).

For each gene, we will then use several enrichment analysis methods, including the Fisher Exact test, GSEA, and others, to assess whether its neighborhood (i.e. other genes and interactions in its proximity within the HGi) is unusually enriched in alterations. As a result, one can plan to study methods that propagate dysregulation/alteration information on the network, which can reduce the dependency on hub size. Since the HGi network includes both directed and adirected interactions, use of individual approaches such as Bayesian Networks or Markov Random Fields is not an option. One can thus explore mixed approaches such as integrating information on two sub-networks, one fully directed and one fully adirected, at alternate time steps as well as using some recent graph-theoretic approaches that were specifically designed for this type of mixed networks. One can define a probability to each gene in the network, that is proportional to the gene's role in tumorigenesis and progression to high-grade tumors and to integrate information sources to compute such probability.

Additional Analyses Supported by the HGi.

Availability of the HGi will allow a rich set of interactomes-based methodologies to be tested on GBM data. For instance, while this research is specifically aimed at the genetic mechanisms that implement and maintain the most aggressive form of glioma, characterized by a mesenchymal signature and phenotype, other important avenues of investigations of the disease are around the dissection of the basic mechanisms of GBM tumorigenesis and the mechanism of action of drugs for the treatment of GBM. Availability of a complete and unbiased HGi, which represents the full complement of genome-wide molecular interactions in the disease, will be a significant tool for additional analyses and we expect that this resource will be heavily used by the community. For instance, the IDEA and MRA can be used to dissect normal vs. tumor phenotypes rather than high-grade vs. low-grade glioma as described in this proposal. Additionally, the approach in EXAMPLES 2-4 and discussed herein can be applied to identify drugs able to implement an apoptotic phenotype in GBM.

### LITERATURE CITED FOR EXAMPLES 2-6

1. 2008. Comprehensive genomic characterization defines human glioblastoma genes and core pathways. Nature 455:1061-8.
2. Aoki, K., G. Meng, K. Suzuki, T. Takashi, Y. Kameoka, K. Nakahara, R. Ishida, and M. Kasai. 1998. RP58 associates with condensed chromatin and mediates a sequence-specific transcriptional repression. J Biol Chem 273:26698-704.
3. Ashburner, M., C. A. Ball, J. A. Blake, D. Botstein, H. Butler, J. M. Chemy, A. P. Davis, K. Dolinski, S. S. Dwight, J. T. Eppig, M. A. Harris, D. P. Hill, L. Issel-Tarver, A. Kasarskis, S. Lewis, J. C. Matese, J. E. Richardson, M. Ringwald, G. M. Rubin, and G. Sherlock. 2000. Gene ontology: tool for the unification of biology. The Gene Ontology Consortium. Nat. Genet. 25:25-9.
4. Bader, G. D., D. Betel, and C. W. Hogue. 2003. BIND: the Biomolecular Interaction Network Database. Nucleic Acids Res 31:248-50.
5. Bajenaru, M. L., J. Donahoe, T. Corral, K. M. Reilly, S. Brophy, A. Pellicer, and D. H. Gutmann. 2001. Neurofibromatosis 1 (NF1) heterozygosity results in a cell-autonomous growth advantage for astrocytes. Glia 33:314-23.
6. Barabasi, A. L., and Z. N. Oltvai. 2004. Network biology: understanding the cell's functional organization. Nat Rev Genet. 5:101-13.
7. Basso, K., A. A. Margolin, G. Stolovitzky, U. Klein, R. Dalla-Favera, and A. Califano. 2005. Reverse engineering of regulatory networks in human B cells. Nat Genet. 37:382-90.
8. Becker, K. G., I. J. Lee, J. W. Nagle, R. D. Canning, A. M. Gado, R. Torres, M. H. Polymeropoulos, P. T. Massa, W. E. Biddison, and P. D. Drew. 1997. C2H2-171: a novel human cDNA representing a developmentally regulated POZ domain/zinc finger protein preferentially expressed in brain. Int J Dev Neurosci 15:891-9.
9. Ben-Dor, A., L. Bruhn, N. Friedman, I. Nachman, M. Schummer, and Z. Yakhini. 2000. Tissue classification with gene expression profiles. J Comput Biol 7:559-83.
10. Beurel, E., and R. S. Jope. 2008. Differential regulation of STAT family members by glycogen synthase kinase-3. J Biol Chem 283:21934-44.
11. Blits, B., B. M. Kitay, A. Farahvar, C. V. Caperton, W. D. Dietrich, and M. B. Bunge. 2005. Lentiviral vector-mediated transduction of neural progenitor cells before implantation into injured spinal cord and brain to detect their migration, deliver neurotrophic factors and repair tissue. Restor Neurol Neurosci 23:313-24.
12. Boyer, L. A., T. I. Lee, M. F. Cole, S. E. Johnstone, S. S. Levine, J. P. Zucker, M. G. Guenther, R. M. Kumar, H. L. Murray, R. G. Jenner, D. K. Gifford, D. A. Melton, R. Jaenisch, and R. A. Young. 2005. Core transcriptional regulatory circuitry in human embryonic stem cells. Cell 122:947-56.
13. Bromberg, J. F., M. H. Wrzeszczynska, G. Devgan, Y. Zhao, R. G. Pestell, C. Albanese, and J. E. Darnell, Jr. 1999. Stat3 as an oncogene. Cell 98:295-303.
14. Bussemaker, H. J., H. Ligand E. D. Siggia. 2001. Regulatory element detection using correlation with expression. Nat Genet. 27:167-71.
15. Chen, P., A. Iavarone, J. Fick, M. Edwards, M. Prados, and M. A. Israel. 1995. Constitutional p53 mutations associated with brain tumors in young adults. Cancer Genet Cytogenet 82:106-15.
16. Consiglio, A., A. Gritti, D. Dolcetta, A. Follenzi, C. Bordignon, F. H. Gage, A. L. Vescovi, and L. Naldini. 2004. Robust in vivo gene transfer into adult mammalian NSCs by lentiviral vectors. Proc Natl Acad Sci USA 101:14835-40.
17. Deng, M., K. Zhang, S. Mehta, T. Chen, and F. Sun. 2003. Prediction of protein function using protein-protein interaction data. J Comput Biol 10:947-60.
18. Doetsch, F., I. Caille, D. A. Lim, J. M. Garcia-Verdugo, and A. Alvarez-Buylla. 1999. Subventricular zone astrocytes are NSCs in the adult mammalian brain. Cell 97:703-16.
19. Ehrich, M., M. R. Nelson, P. Stanssens, M. Zabeau, T. Liloglou, G. Xinarianos, C. R. Cantor, J. K. Field, and D. van den Boom. 2005. Quantitative high-throughput analysis of DNA methylation patterns by base-specific cleavage and mass spectrometry. Proc Natl Acad Sci USA 102:15785-90.
20. Ergun, A., C. A. Lawrence, M. A. Kohanski, T. A. Brennan, and J. J. Collins. 2007. A network biology approach to prostate cancer. Mol Syst Biol 3:82.
21. Frank, D. A. 2007. STAT3 as a central mediator of neoplastic cellular transformation. Cancer Lett 251:199-210.
22. Freije, W. A., F. E. Castro-Vargas, Z. Fang, S. Horvath, T. Cloughesy, L. M. Liau, P. S. Mischel, and S. F. Nelson. 2004. Gene expression profiling of gliomas strongly predicts survival. Cancer Res 64:6503-10.
23. Fuks, F., W. A. Burgers, N. Godin, M. Kasai, and T. Kouzarides. 2001. Dnmt3a binds deacetylases and is recruited by a sequence-specific repressor to silence transcription. Embo J 20:2536-44.
24. Gasch, A. P., P. T. Spellman, C. M. Kao, O. Carmel-Harel, M. B. Eisen, G. Storz, D. Botstein, and P. O. Brown. 2000. Genomic expression programs in the response of yeast cells to environmental changes. Mol Biol Cell 11:4241-57.
25. Godard, S., G. Getz, M. Delorenzi, P. Farmer, H. Kobayashi, 1. Desbaillets, M. Nozaki, A. C. Diserens, M. F. Hamou, P. Y. Dietrich, L. Regli, R. C. Janzer, P. Bucher, R. Stupp, N. de Tribolet, E. Domany, and M. E. Hegi. 2003. Classification of human astrocytic gliomas on the basis of gene expression: a correlated group of genes with angiogenic activity emerges as a strong predictor of subtypes. Cancer Res 63:6613-25.
26. Goh, K. I., M. E. Cusick, D. Valle, B. Childs, M. Vidal, and A. L. Barabasi. 2007. The human disease network. Proc Natl Acad Sci USA 104:8685-90.
27. Hart, G. T., A. K. Ramani, and E. M. Marcotte. 2006. How complete are current yeast and human protein-interaction networks? Genome Biol 7:120.
28. Hart, K. C., S. C. Robertson, and D. J. Donoghue. 2001. Identification of tyrosine residues in constitutively activated fibroblast growth factor receptor 3 involved in mitogenesis, Stat activation, and phosphatidylinositol 3-kinase activation. Mol Biol Cell 12:931-42.
29. Hermjakob, H., L. Montecchi-Palazzi, C. Lewington, S. Mudali, S. Kerrien, S. Orchard, M. Vingron, B. Roechert, P. Roepstorff, A. Valencia, H. Margalit, J. Armstrong, A. Bairoch, G. Cesareni, D. Sherman, and R. Apweiler. 2004. IntAct: an open source molecular interaction database. Nucleic Acids Res 32:D452-5.
30. Hopkins, A. L., and C. R. Groom. 2002. The druggable genome. Nat Rev Drug Discov 1:727-30.
31. Iavarone, A., E. R. King, X. M. Dai, G. Leone, E. R. Stanley, and A. Lasorella. 2004. Retinoblastoma promotes definitive erythropoiesis by repressing Id2 in fetal liver macrophages. Nature 432:1040-5.
32. Iavarone, A., K. K. Matthay, T. M. Steinkirchner, and M. A. Israel. 1992. Germ-line and somatic p53 gene mutations in multifocal osteogenic sarcoma. Proc Natl Acad Sci USA 89:4207-9.
33. Ingenuity Systems, I. www.ingenuity.com.
34. Inghirami, G., R. Chiarle, W. J. Simmons, R. Piva, K. Schlessinger, and D. E. Levy. 2005. New and old functions of STAT3: a pivotal target for individualized treatment of cancer. Cell Cycle 4:1131-3.
35. Jansen, R., N. Lan, J. Qian, and M. Gerstein. 2002. Integration of genomic datasets to predict protein complexes in yeast. J Struct Funct Genomics 2:71-81.
36. Jansen, R., H. Yu, D. Greenbaum, Y. Kluger, N. J. Krogan, S. Chung, A. Emili, M. Snyder, J. F. Greenblatt, and M. Gerstein. 2003. A Bayesian networks approach for predicting protein-protein interactions from genomic data. Science 302:449-53.
37. Kalir, S., S. Mangan, and U. Alon. 2005. A coherent feed-forward loop with a SUM input function prolongs flagella expression in Escherichia coli. Mol Syst Biol 1:2005 0006.
38. Kwon, C. H., X. Zhu, J. Zhang, L. L. Knoop, R. Tharp, R. J. Smeyne, C. G. Eberhart, P. C. Burger, and S. J. Baker. 2001. Pten regulates neuronal soma size: a mouse model of Lhermitte-Duclos disease. Nat Genet. 29:404-11.
39. La Porta, C. A., C. Franchi, and R. Comolli. 1998. c-PKC-dependent modulation of plasma fibrinogen levels during the acute-phase response in young and old rats. Mech Ageing Dev 103:317-26.
40. Lamb, J., E. D. Crawford, D. Peck, J. W. Modell, I. C. Blat, M. J. Wrobel, J. Lerner, J. P. Brunet, A. Subramanian, K. N. Ross, M. Reich, H. Hieronymus, G. Wei, S. A. Armstrong, S. J. Haggarty, P. A. Clemons, R. Wei, S. A. Carr, E. S. Lander, and T. R. Golub. 2006. The Connectivity Map: using gene-expression signatures to connect small molecules, genes, and disease. Science 313:1929-35.
41. Lasorella, A., M. Noseda, M. Beyna, Y. Yokota, and A. Iavarone. 2000. Id2 is a retinoblastoma protein target and mediates signalling by Myc oncoproteins. Nature 407:592-8.
42. Lee, J., S. Kotliarova, Y. Kotliarov, A. L1, Q. Su, N. M. Donin, S. Pastorino, B. W. Purow, N. Christopher, W. Zhang, J. K. Park, and H. A. Fine. 2006. Tumor stem cells derived from glioblastomas cultured in bFGF and EGF more closely mirror the phenotype and genotype of primary tumors than do serum-cultured cell lines. Cancer Cell 9:391-403.
43. Lee, J. P., M. Jeyakumar, R. Gonzalez, H. Takahashi, P. J. Lee, R. C. Baek, D. Clark, H. Rose, G. Fu, J. Clarke, S. McKercher, J. Meerloo, F. J. Muller, K. I. Park, T. D. Butters, R. A. Dwek, P. Schwartz, G. Tong, D. Wenger, S. A. Lipton, T. N. Seyfried, F. M. Platt, and E. Y. Snyder. 2007. Stem cells act through multiple mechanisms to benefit mice with neurodegenerative metabolic disease. Nat Med 13:439-47.
44. Lee, T. I., N. J. Rinaldi, F. Robert, D. T. Odom, Z. Bar-Joseph, G. K. Gerber, N. M. Hannett, C. T. Harbison, C. M. Thompson, I. Simon, J. Zeitlinger, E. G. Jennings, H. L. Murray, D. B. Gordon, B. Ren, J. J. Wyrick, J. B. Tagne, T. L. Volkert, E. Fraenkel, D. K. Gifford, and R. A. Young. 2002. Transcriptional regulatory networks in Saccharomyces cerevisiae. Science 298:799-804.
45. Lefebvre, C., W. Lim, K. Basso, R. Dalla Favera, and A. Califano. 2006. A context-specific network of protein-DNA and protein-protein interactions reveals new regulatory motifs in human B cells. RECOMB Satellite Workshop on Systems Biology, San Diego, December 2006. Also in press in Lecture Notes in Bioinformatics, Springer Verlag.
46. Legler, J. M., L. A. Gloeckler Ries, M. A. Smith, J. L. Warren, E. F. Heineman, R. S. Kaplan, and M. S. Linet. 2000. RESPONSE: re: brain and other central nervous system cancers: recent trends in incidence and mortality. J Natl Cancer Inst 92:77 A-8.
47. Liang, Y., M. Diehn, N. Watson, A. W. Bollen, K. D. Aldape, M. K. Nicholas, K. R. Lamborn, M. S. Berger, D. Botstein, P. O. Brown, and M. A. Israel. 2005. Gene expression profiling reveals molecularly and clinically distinct subtypes of glioblastoma multiforme. Proc Natl Acad Sci USA 102:5814-9.
48. Lim, W. K., and A. Califano. 2007. Presented at the RECOMB Regulatory Genomics, Boston, October 11-13.
49. Lim, W. K., E. Lyashenko, and A. Califano. 2009. Master regulators used as breast caqncer metastasis classifier. Pac Symp Biocomput 14:504-515.
50. Linding, R., L. J. Jensen, G. J. Ostheimer, M. A. van Vugt, C. Jorgensen, I. M. Miron, F. Diella, K. Colwill, L. Taylor, K. Elder, P. Metalnikov, V. Nguyen, A. Pasculescu, J. Jin, J. G. Park, L. D. Samson, J. R. Woodgett, R. B. Russell, P. Bork, M. B. Yaffe, and T. Pawson. 2007. Systematic discovery of in vivo phosphorylation networks. Cell 129:1415-26.
51. Ludwig, T., P. Fisher, S. Ganesan, and A. Efstratiadis. 2001. Tumorigenesis in mice carrying a truncating Brcal mutation. Genes Dev 15:1188-93.
52. Ludwig, T., P. Fisher, V. Murty, and A. Efstratiadis. 2001. Development of mammary adenocarcinomas by tissue-specific knockout of Brca2 in mice. Oncogene 20:3937-48.
53. Malatesta, P., M. A. Hack, E. Hartfuss, H. Kettenmann, W. Klinkert, F. Kirchhoff, and M. Gotz. 2003. Neuronal or glial progeny: regional differences in radial glia fate. Neuron 37:751-64.
54. Malatesta, P., E. Hartfuss, and M. Gotz. 2000. Isolation of radial glial cells by fluorescent-activated cell sorting reveals a neuronal lineage. Development 127:5253-63.
55. Mani, K. M., C. Lefebvre, K. Wang, W. K. Lim, K. Basso, R. Dalla Favera, and A. Califano. 2007. A Systems biology approach to prediction of oncogenes and perturbation targets in B cell lymphomas. Molecular Systems Biology 4:169-179, 2008.
56. Margolin, A. A., I. Nemenman, K. Basso, C. Wiggins, G. Stolovitzky, R. Dalla Favera, and A. Califano. 2006. ARACNE: an algorithm for the reconstruction of gene regulatory networks in a mammalian cellular context. BMC Bioinformatics 7 Suppl 1:S7.
57. Margolin, A. A., T. Palomero, P. Sumazin, A. Califano, A. A. Ferrando, and G. Stolovitzky. 2009. ChIP-on-chip significance analysis reveals large-scale binding and regulation by human TF oncogenes. Proc Natl Acad Sci USA 106:244-9.
58. Margolin, A. A., K. Wang, W. K. Lim, M. Kustagi, I. Nemenman, and A. Califano. 2006. Reverse engineering cellular networks. Nat Protoc 1:662-71.
59. Marino, S., M. Vooijs, H. van Der Gulden, J. Jonkers, and A. Berns. 2000. Induction of medulloblastomas in p53-null mutant mice by somatic inactivation of Rb in the external granular layer cells of the cerebellum. Genes Dev 14:994-1004.
60. Matsuo, R., W. Ochiai, K. Nakashima, and T. Taga. 2001. A new expression cloning strategy for isolation of substrate-specific kinases by using phosphorylation site-specific antibody. J Immunol Methods 247:141-51.
61. Matys, V., E. Fricke, R. Geffers, E. Gossling, M. Haubrock, R. Hehl, K. Hornischer, D. Karas, A. E. Kel, O. V. Kel-Margoulis, D. U. Kloos, S. Land, B. Lewicki-Potapov, H. Michael, R. Munch, I. Reuter, S. Rotert, H. Saxel, M. Scheer, S. Thiele, and E. Wingender. 2003. TRANSFAC: transcriptional regulation, from patterns to profiles. Nucleic Acids Res 31:374-8.
62. Merkle, F. T., A. D. Tramontin, J. M. Garcia-Verdugo, and A. Alvarez-Buylla. 2004. Radial glia give rise to adult NSCs in the subventricular zone. Proc Natl Acad Sci USA 101:17528-32.
63. Mewes, H. W., D. Frishman, K. F. Mayer, M. Munsterkotter, O, Noubibou, P. Pagel, T. Rattei, M. Oesterheld, A. Ruepp, and V. Stumpflen. 2006. MIPS: analysis and annotation of proteins from whole genomes in 2005. Nucleic Acids Res 34:D169-72.
64. Mulder, N. J., R. Apweiler, T. K. Attwood, A. Bairoch, A. Bateman, D. Binns, P. Bork, V. Buillard, L. Cerutti, R. Copley, E. Courcelle, U. Das, L. Daugherty, M. Dibley, R. Finn, W. Fleischmann, J. Gough, D. Haft, N. Hulo, S. Hunter, D. Kahn, A. Kanapin, A. Kejariwal, A. Labarga, P. S. Langendijk-Genevaux, D. Lonsdale, R. Lopez, I. Letunic, M. Madera, J. Maslen, C. McAnulla, J. McDowall, J. Mistry, A. Mitchell, A. N. Nikolskaya, S. Orchard, C. Orengo, R. Petryszak, J. D. Selengut, C. J. Sigrist, P. D. Thomas, F. Valentin, D. Wilson, C. H. Wu, and C. Yeats. 2007. New developments in the InterPro database. Nucleic Acids Res 35:D224-8.
65. Niehof, M., S. Kubicka, L. Zender, M. P. Manns, and C. Trautwein. 2001. Autoregulation enables different pathways to control CCAAT/enhancer binding protein beta (C/EBP beta) transcription. J Mol Biol 309:855-68.
66. Nigro, J. M., A. Misra, L. Zhang, I. Smirnov, H. Colman, C. Griffin, N. Ozburn, M. Chen, E. Pan, D. Koul, W. K. Yung, B. G. Feuerstein, and K. D. Aldape. 2005. Integrated array-comparative genomic hybridization and expression array profiles identify clinically relevant molecular subtypes of glioblastoma. Cancer Res 65:1678-86.
67. Noctor, S. C., A. C. Flint, T. A. Weissman, R. S. Dammerman, and A. R. Kriegstein. 2001. Neurons derived from radial glial cells establish radial units in neocortex. Nature 409:714-20.
68. Odom, D. T., R. D. Dowell, E. S. Jacobsen, L. Nekludova, P. A. Rolfe, T. W. Danford, D. K. Gifford, E. Fraenkel, G. I. Bell, and R. A. Young. 2006. Core transcriptional regulatory circuitry in human hepatocytes. Mol Syst Biol 2:2006 0017.
69. Orlow, I., A. Iavarone, S. J. Crider-Miller, F. Bonilla, E. Latres, M. H. Lee, W. L. Gerald, J. Massague, B. E. Weissman, and C. Cordon-Cardo. 1996. Cyclin-dependent kinase inhibitor p57KIP2 in soft tissue sarcomas and Wilms'tumors. Cancer Res 56:1219-21.
70. Palomero, T., W. K. Lim, D. T. Odom, M. L. Sulis, P. J. Real, A. Margolin, K. C. Barnes, J. O'Neil, D. Neuberg, A. P. Weng, J. C. Aster, F. Sigaux, J. Soulier, A. T. Look, R. A. Young, A. Califano, and A. A. Ferrando. 2006. NOTCH1 directly regulates c-MYC and activates a feed-forward-loop transcriptional network promoting leukemic cell growth. Proc Natl Acad Sci USA 103:18261-6.
71. Park, J. 1., C. J. Strock, D. W. Ball, and B. D. Nelkin. 2003. The Ras/Raf/MEK/extracellular signal-regulated kinase pathway induces autocrine-paracrine growth inhibition via the leukemia inhibitory factor/JAK/STAT pathway. Mol Cell Biol 23:543-54.
72. Park, K. I., M. A. Hack, J. Ourednik, B. Yandava, J. D. Flax, P. E. Stieg, S. Gullans, F. E. Jensen, R. L. Sidman, V. Ourednik, and E. Y. Snyder. 2006. Acute injury directs the migration, proliferation, and differentiation of solid organ stem cells: evidence from the effect of hypoxia-ischemia in the CNS on clonal “reporter” NSCs. Exp Neurol 199:156-78.
73. Park, Y. J., E. S. Park, M. S. Kim, T. Y. Kim, H. S. Lee, S. Lee, I. S. Jang, M. Shong, D. J. Park, and B. Y. Cho. 2002. Involvement of the protein kinase C pathway in thyrotropin-induced STAT3 activation in FRTL-5 thyroid cells. Mol Cell Endocrinol 194:77-84.
74. Parker, M. A., J. K. Anderson, D. A. Corliss, V. E. Abraria, R. L. Sidman, K. I. Park, Y. D. Teng, D. A. Cotanche, and E. Y. Snyder. 2005. Expression profile of an operationally-defined neural stem cell clone. Exp Neurol 194:320-32:
75. Pelloski, C. E., A. Mahajan, M. Maor, E. L. Chang, S. Woo, M. Gilbert, H. Colman, H. Yang, A. Ledoux, H. Blair, S. Passe, R. B. Jenkins, and K. D. Aldape. 2005. YKL-40 expression is associated with poorer response to radiation and shorter overall survival in glioblastoma. Clin Cancer Res 11:3326-34.
76. Peri, S., J. D. Navarro, R. Amanchy, T. Z. Kristiansen, C. K. Jonnalagadda, V. Surendranath, V. Niranjan, B. Muthusamy, T. K. Gandhi, M. Gronborg, N. Ibarrola, N. Deshpande, K. Shanker, H. N. Shivashankar, B. P. Rashmi, M. A. Ramya, Z. Zhao, K. N. Chandrika, N. Padma, H. C. Harsha, A. J. Yatish, M. P. Kavitha, M. Menezes, D. R. Choudhury, S. Suresh, N. Ghosh, R. Saravana, S. Chandran, S. Krishna, M. Joy, S. K. Anand, V. Madavan, A. Joseph, G. W. Wong, W. P. Schiemann, S, N. Constantinescu, L. Huang, R. Khosravi-Far, H. Steen, M. Tewari, S. Ghaffari, G. C. Blobe, C. V. Dang, J. G. Garcia, J. Pevsner, O. N. Jensen, P. Roepstorff, K. S. Deshpande, A. M. Chinnaiyan, A. Hamosh, A. Chakravarti, and A. Pandey. 2003. Development of human protein reference database as an initial platform for approaching systems biology in humans. Genome Res 13:2363-71.
77. Phillips, H. S., S. Kharbanda, R. Chen, W. F. Forrest, R. H. Soriano, T. D. Wu, A. Misra, J. M. Nigro, H. Colman, L. Soroceanu, P. M. Williams, Z. Modrusan, B. G. Feuerstein, and K. Aldape. 2006. Molecular subclasses of high-grade glioma predict prognosis, delineate a pattern of disease progression, and resemble stages in neurogenesis. Cancer Cell 9:157-73.
78. Piccirillo, S. G., B. A. Reynolds, N. Zanetti, G. Lamorte, E. Binda, G. Broggi, H. Brem, A. Olivi, F. Dimeco, and A. L. Vescovi. 2006. Bone morphogenetic proteins inhibit the tumorigenic potential of human brain tumour-initiating cells. Nature 444:761-5.
79. Ramji, D. P., and P. Foka. 2002. CCAAT/enhancer-binding proteins: structure, function and regulation. Biochem J 365:561-75.
80. Rasmussen, S. A., Q. Yang, and J. M. Friedman. 2001. Mortality in neurofibromatosis 1: an analysis using U.S. death certificates. Am J Hum Genet. 68:1110-8.
81. Ridet, J. L., S. K. Malhotra, A. Privat, and F. H. Gage. 1997. Reactive astrocytes: cellular and molecular cues to biological function. Trends Neurosci 20:570-7.
82. Rual, J. F., K. Venkatesan, T. Hao, T. Hirozane-Kishikawa, A. Dricot, N. L1, G. F. Berriz, F. D. Gibbons, M. Dreze, N. Ayivi-Guedehoussou, N. Klitgord, C. Simon, M. Boxem, S. Milstein, J. Rosenberg, D. S. Goldberg, L. V. Zhang, S. L. Wong, G. Franklin, S. Li, J. S. Albala, J. Lim, C. Fraughton, E. Llamosas, S. Cevik, C. Bex, P. Lamesch, R. S. Sikorski, J. Vandenhaute, H. Y. Zoghbi, A. Smolyar, S. Bosak, R. Sequerra, L. Doucette-Stamm, M. E. Cusick, D. E. Hill, F. P. Roth, and M. Vidal. 2005. Towards a proteome-scale map of the human protein-protein interaction network. Nature 437:1173-8.
83. Rzhetsky, A., I. Iossifov, T. Koike, M. Krauthammer, P. Kra, M. Morris, H. Yu, P. A. Duboue, W. Weng, W. J. Wilbur, V. Hatzivassiloglou, and C. Friedman. 2004. Gene Ways: a system for extracting, analyzing, visualizing, and integrating molecular pathway data. J Biomed Inform 37:43-53.
84. Smith, A. D., P. Sumazin, D. Das, and M. Q. Zhang. 2005. Mining ChIP-chip data for TF and cofactor binding sites. Bioinformatics 21 Suppl 1:1403-12.
85. Smith, A. D., P. Sumazin, Z. Xuan, and M. Q. Zhang. 2006. DNA motifs in human and mouse proximal promoters predict tissue-specific expression. Proc Natl Acad Sci USA 103:6275-80.
86. Smith, A. D., P. Sumazin, and M. Q. Zhang. 2005. Identifying tissue-selective TF binding sites in vertebrate promoters. Proc Natl Acad Sci USA 102:1560-5.
87. Smith, A. D., P. Sumazin, and M. Q. Zhang. 2007. Tissue-specific regulatory elements in mammalian promoters. Mol Syst Biol 3:73.
88. Sosinsky, A., B. Honig, R. S. Mann, and A. Califano. 2007. Discovering transcriptional regulatory regions in Drosophila by a nonalignment method for phylogenetic footprinting. Proc Natl Acad Sci USA 104:6305-10.
89. Stanssens, P., M. Zabeau, G. Meersseman, G. Remes, Y. Gansemans, N. Storm, R. Hartmer, C. Honisch, C. P. Rodi, S. Bocker, and D. van den Boom. 2004. High-throughput MALDI-TOF discovery of genomic sequence polymorphisms. Genome Res 14:126-33.
90. Steinman, R. A., A. Wentzel, Y. Lu, C. Stehle, and J. R. Grandis. 2003. Activation of Stat3 by cell confluence reveals negative regulation of Stat3 by cdk2. Oncogene 22:3608-15.
91. Stelzl, U., U. Worm, M. Lalowski, C. Haenig, F. H. Brembeck, H. Goehler, M. Stroedicke, M. Zenkner, A. Schoenherr, S. Koeppen, J. Timm, S. Mintzlaff, C. Abraham, N. Bock, S. Kietzmann, A. Goedde, E. Toksoz, A. Droege, S. Krobitsch, B. Korn, W. Birchmeier, H. Lehrach, and E. E. Wanker. 2005. A human protein-protein interaction network: a resource for annotating the proteome. Cell 122:957-68.
92. Subramanian, A., P. Tamayo, V. K. Mootha, S. Mukherjee, B. L. Ebert, M. A. Gillette, A. Paulovich, S. L. Pomeroy, T. R. Golub, E. S. Lander, and J. P. Mesirov. 2005. Gene set enrichment analysis: a knowledge-based approach for interpreting genome-wide expression profiles. Proc Natl Acad Sci USA 102:15545-50.
93. Sun, S., and B. M. Steinberg. 2002. PTEN is a negative regulator of STAT3 activation in human papillomavirus-infected cells. J Gen Virol 83:1651-8.
94. Szulc, J., M. Wiznerowicz, M. O, Sauvain, D. Trono, and P. Aebischer. 2006. A versatile tool for conditional gene expression and knockdown. Nat Methods 3:109-16.
95. Takashima, Y., T. Era, K. Nakao, S. Kondo, M. Kasuga, A. G. Smith, and S, Nishikawa. 2007. Neuroepithelial cells supply an initial transient wave of MSC differentiation. Cell 129:1377-88.
96. Tegner, J., M. K. Yeung, J. Hasty, and J. J. Collins. 2003. Reverse engineering gene networks: integrating genetic perturbations with dynamical modeling. Proc Natl Acad Sci USA 100:5944-9.
97. Tramontin, A. D., J. M. Garcia-Verdugo, D. A. Lim, and A. Alvarez-Buylla. 2003. Postnatal development of radial glia and the ventricular zone (VZ): a continuum of the neural stem cell compartment. Cereb Cortex 13:580-7.
98. Tso, C. L.; P. Shintaku, J. Chen, Q. Liu, J. Liu, Z. Chen, K. Yoshimoto, P. S. Mischel, T. F. Cloughesy, L. M. Liau, and S. F. Nelson. 2006. Primary glioblastomas express mesenchymal stem-like properties. Mol Cancer Res 4:607-19.
99. Vescovi, A. L., R. Galli, and B. A. Reynolds. 2006. Brain tumour stem cells. Nat Rev Cancer 6:425-36.
100. Wang, K., M. Alvarez, B. Bisikirska, R. Linding, K. Basso, R. Dalla Favera, and A. Califano. 2009. Dissecting the Interface Between Signaling and Transcriptional Regulation in Human B Cells. Pacific Symposium on Biocomputing 14:264-275.
101. Wang, K., N. Banerjee, A. Margolin, I. Nemenman, and A. Califano. 2006. Genome-wide discovery of modulators of transcriptional interactions in human B lymphocytes. Lecture Notes in Computer Science 3909:348-362.
102. Wang, K., M. Saito, I. Nemenman, K. Basso, A. A. Margolin, U. Klein, R. Dalla Favera, and A. Califano. 2008. Genome-wide identification of transcriptional network modulators in human B cells. submitted.
103. Wang, L., T. Kurosaki, and S. J. Corey. 2007. Engagement of the B-cell antigen receptor activates STAT through Lyn in a Jak-independent pathway. Oncogene 26:2851-9.
104. Weinstein, I. B. 2002. Cancer. Addiction to oncogenes—the Achilles heal of cancer. Science 297:63-4.
105. Wurmser, A. E., K. Nakashima, R. G. Summers, N. Toni, K. A. D'Amour, D. C. Lie, and F. H. Gage. 2004. Cell fusion-independent differentiation of NSCs to the endothelial lineage. Nature 430:350-6.
106. Yang, J., S. A. Mani, J. L. Donaher, S. Ramaswamy, R. A. Itzykson, C. Come, P. Savagner, I. Gitelman, A. Richardson, and R. A. Weinberg. 2004. Twist, a master regulator of morphogenesis, plays an essential role in tumor metastasis. Cell 117:927-39.
107. Yin, F., P. L1, M. Zheng, L. Chen, Q. Xu, K. Chen, Y. Y. Wang, Y. Y. Zhang, and C. Han. 2003. Interleukin-6 family of cytokines mediates isoproterenol-induced delayed STAT3 activation in mouse heart. J Biol Chem 278:21070-5.
108. Yu, H., and M. Gerstein. 2006. Genomic analysis of the hierarchical structure of regulatory networks. Proc Natl Acad Sci USA 103:14724-31.
109. Zardo, G., M. I. Tiirikainen, C. Hong, A. Misra, B. G. Feuerstein, S. Volik, C. C. Collins, K. R. Lamborn, A. Bollen, D. Pinkel, D. G. Albertson, and J. F. Costello. 2002. Integrated genomic and epigenomic analyses pinpoint biallelic gene inactivation in tumors. Nat Genet. 32:453-8.
110. Zeller, K. 1., A. G. Jegga, B. J. Aronow, K. A. O'Donnell, and C. V. Dang. 2003. An integrated database of genes responsive to the Myc oncogenic TF: identification of direct genomic targets. Genome Biol 4:R69.
111. Zhu, X., M. Gerstein, and M. Snyder. 2007. Getting connected: analysis and principles of biological networks. Genes Dev 21:1010-24.
112. Zhuo, L., M. Theis, 1. Alvarez-Maya, M. Brenner, K. Willecke, and A. Messing. 2001. hGFAP-cre transgenic mice for manipulation of glial and neuronal function in vivo. Genesis 31:85-94.

### Example 7

### A Transcriptional Module Synergistically Initiates and Maintains Mesenchymal Transformation in the Brain

Using a combination of cellular-network reverse engineering algorithms and experimental validation assays, a small transcriptional module was identified, including six transcription factors (TFs), that synergistically regulates the mesenchymal signature of malignant glioma. This is a poorly understood molecular phenotype, never observed in normal neural tissue (A 1-3; each herein incorporated by reference in its entirety). It represents the hallmark of tumor aggressiveness in high-grade glioma, and its upstream regulation is so far unknown (A1). Overall, the newly discovered transcriptional module regulates >74% of the signature genes, while two of its TFs (Stat3 and C/EBPβ) display features of initiators and master regulators of mesenchymal transformation. Ectopic co-expression of Stat3 and C/EBPβ is sufficient to reprogram neural stem cells along the aberrant mesenchymal lineage, while simultaneously suppressing genes associated with the normal neuronal state (pro-neural signature). These effects promote tumor formation in the mouse and endow neural stem cells with the phenotypic hallmarks of the mesenchymal state (migration and invasion). Silencing the two TFs in human high grade glioma-derived stem cells and glioma cell lines leads to the collapse of the mesenchymal signature with corresponding reduction in tumor aggressiveness. In human tumor samples, combined expression of Stat3 and C/EBPβ correlates with mesenchymal differentiation of primary glioma and it is a powerful predictor of poor clinical outcome. Taken together, these results reveal that synergistic activation of a small transcriptional module, inferred using a systems biology approach, is necessary and sufficient to reprogram neural stem cells towards a transformed mesenchymal state. This provides the first experimentally validated computational approach to infer master transcriptional regulators from signatures of human cancer.

To discover TFs causally linked to the expression of the MGES+ signature the conventional paradigm of microarray expression profile based cancer research was inverted. Rather than asking which genes are part of the MGES+ signature, a computationally inferred, genomewide transcriptional interaction map was interrogated to identify which TFs in the human genome can induce its overexpression in vivo. Such an unbiased, genome-wide approach was not previously attempted because knowledge of the transcriptional regulatory interactions within a specific cellular phenotype is extraordinarily sparse, especially in a mammalian context. Thus, only a handful of candidate TFs can be previously interrogated in this fashion and only after obtaining large-scale binding and functional assays in the specific cellular context of interest (A10; herein incorporated by reference in its entirety). Recently, however, reverse engineering approaches have been pioneered for the genome-wide inference of regulatory networks in mammalian cells (A11, A12; herein incorporated by reference in its entirety) and have been applied to the identification of lesions associated with the dysregulation of tumor-related pathways (A13; herein incorporated by reference in its entirety). It has been reasoned that these algorithms can allow one to use causal logic rather than statistical associations (A14, 15; herein incorporated by reference in its entirety) towards the identification of master regulators of the MGES+ signature. It is shown that the integration of multiple reverse engineering algorithms, based on expression profile and sequence data from glioma patients, produces highaccuracy maps of the regulatory relationships in normal and transformed neural cells. These computational findings were biochemically validated and subsequently used to identify the transcriptional events responsible for initiation and maintenance of the mesenchymal phenotype of high-grade glioma.

Specifically, computational, functional, and chromatin immunoprecipitation (ChIP) experiments motivated by the inferred regulatory network topology point to two TFs (Stat3 and C/EBPβ) as master regulators of the mesenchymal signature of human glioma. Ectopic co-expression of the two factors in neural stem cells is sufficient to initiate expression of the mesenchymal set of genes, suppress proneural genes and trigger invasion and a malignant mesenchymal phenotype in the mouse. Conversely, silencing of these TFs depletes glioma stem cells and cell lines of mesenchymal attributes and greatly impairs their ability to invade. Most notably, independent immunohistochemistry experiments in 62 human glioma specimens show that concurrent expression of Stat3 and C/EBPβ is significantly associated to the expression of mesenchymal proteins and is an accurate predictor of poorest outcome in glioma patients.

Methods

ARACNe Network Reconstruction.

ARACNe (Algorithm for the Reconstruction of Accurate Cellular Networks), an information-theoretic algorithm for inferring transcriptional interactions, was used to identify a repertoire of candidate transcriptional regulators of the MGES genes. Expression profiles used in the analysis were previously characterized using Affymetrix HU-133A microarrays and preprocessed by MAS 5.0 normalization procedure 1. First, candidate interactions between a TF (x) and its potential target (y) are identified by computing pairwise mutual information, MI[x; y], using a Gaussian kernel estimator (A39) and by thresholding the mutual information based on the null-hypothesis of statistical independence (p<0.05 Bonferroni corrected for the number of tested pairs). Then, indirect interactions are removed using the data processing inequality, a well known property of the mutual information. For each TFtarget pair (x, y) we considered a path through any other TF (z) and remove any interaction such that MI[x; y]<min(MI[x; z], MI[y; z]).

Stepwise Linear Regression (SLR) Analysis.

A regulatory program for each MGES gene was computed as follows: the log₂ expression of the i-thMGES gene was considered as the response variable and the log₂-expression of the TFs as the explanatory variables in the linear model log xᵢ=Σαᵢⱼ log fⱼ+βᵢⱼ (A24). Here, fⱼ represents the expression of the j-th TF in the model and the (αᵢⱼ, βᵢⱼ) are linear coupling coefficients computed by standard regression analysis. TFs are iteratively added to the model, by choosing each time the one producing the smallest relative error E=Σ|xᵢ−xᵢ₀|/xᵢ₀ between predicted and observed target expression. This is repeated until the decrease in relative error is no longer statistically significant. To avoid excessive multiple hypothesis testing correction, TFs were chosen only among the following: (a) the 55 inferred by ARACNe at FDR <0.05 and (b) TFs whose DNA binding signature was significantly enriched in the proximal promoter of the MGES genes and that are expressed in the dataset, based on the coefficient of variation (CV≧0.5). Then, for each TF, the number of MGES target programs it contributed to and the average value of the coupling coefficient were counted.

Cell Lines and Cell Culture Conditions.

SNB75, SNB19, 293T and Rat1 cell lines were grown in DMEM plus 10% Fetal Bovine Serum (FBS, Gibco/BRL). GBM-derived BTSCs were grown as neurospheres in NBE media consisting of Neurobasal media (Invitrogen), N2 and B27 supplements (0.5× each; Invitrogen), human recombinant bFGF and EGF (50 ng/ml each; R&D Systems). Murine neural stem cells (mNSCs) (from an early passage of clone C17.2) (A27-29; each herein incorporated by reference in its entirety) were cultured in DMEM plus 10% Fetal Bovine Serum (FBS), 5% Horse serum (HS, Gibco/BRL) and 1% L-Glutamine (Gibco/BRL). Subclones are extremely easy to make from this line of mNSCs. For such stable mNSC subclones, 10% DMEM Tet system Approved (Clontech) was used.

To generate stable mNSC subclones, the cells were transfected with pBigibHLH-B2-FLAG, pcDNA6-V5-C/EBPβ and pBabe-FLAG-Stat3C using Lipofectamine 2000 (Invitrogen), according to the manufacturer's instructions. Cells were selected with 3 μg/ml Puromycin (Sigma), 6.5 μg/ml Blasticydin (SIGMA), and 300 μg/ml Hygromycin B (Invitrogen). Single clones were isolated and analyzed for the expression of the recombinant proteins using monoclonal antobodies anti-FLAG (M2, SIGMA) and anti-V5 (Invitrogen). bHLH-B2 expression was induced with 2 μg/ml Doxyxycline (Sigma) for 24 hrs. To induce neuronal differentiation, mNSCs were grown in 0.5% Horse serum for 10 days.

Brain tumor stem cells were grown as neurospheres in Neurobasal medium (Invitrogen) containing N2 and B27 supplements and 50 ng/ml of EGF and basic FGF. Cells were transduced with lentiviruses expressing shRNA for Stat3 and C/EBPβ or the empty vector and were analyzed 6 days after infection.

Plasmid Constructs.

pcDNA6-V5-C/EBPβ was constructed as follows. cDNA encoding murine C/EBPβ was amplified from pcDNA3.1-mC/EBPβ using the following primers: C/EBPβ-EcoRI-for (5′-GCCTTGGAATTCATGGAAGTGGCCAACTTC-3′; SEQ ID NO: 1) and C/EBPβ-XbaI-rev (5′-GCCTTGTCTAGACGGCAGTGACCGGCCGAGGC-3′; SEQ ID NO: 2). The amplified sequence was digested with EcoRI and XbaI and subcloned into pcDNA6 in frame with V5 tag. To create pBig21-b-HLH-B2-FLAG, pcDNA3.1-bHLHB2-FLAG was digested with EcoRI and subcloned into pBig21. pBabe-Flag-Stat3C, expressing a constitutive active form of murine Stat3.

Chromatin Immunoprecipitation (ChIP).

Chromatin immunoprecipitaion was performed as described in (A40; herein incorporated by reference in its entirety). SNB75 cells were cross-linked with 1% formaldehyde for 10 min and stopped with 0.125 M glycine for 5 min. Fixed cells were washed in PBS and harvested in sodium dodecyl sulfate buffer. After centrifugation, cells were resuspended in ice-cold immunoprecipitation buffer and sonicated to obtain fragments of 500-1000 pb. Lysates were centrifuged at full speed and the supernatant was precleared with Protein A/G beads (Santa Cruz) and incubated at 4° C. overnight with 1 μg of polyclonal antibody specific for C/EBPβ (sc-150, Santa Cruz), Stat3 (sc-482, Santa Cruz), FosL2 (Fra2, sc-604, Santa Cruz), bHLH-B2 (A300-649A, BETHYL laboratories), or 1 μg of normal rabbit immunoglobulins (Santa Cruz). The immunocomplexes were recovered by incubating the lysates with protein A/G for 1 additional hour at 4° C. After washing, the immunocomplexes were eluted, reverse cross-linked and DNA was recovered by phenolchloroform extraction and ethanol precipitation. DNA was eluted in 200 μl of water and 1 μl was analyzed by PCR with Platinum Taq (Invitrogen).

A modified protocol was developed for the ChIP assays testing interaction of TFs with the promoters of mesenchymal genes in primary GBM samples. Briefly, 30 mg of frozen GBM samples per antibody were chopped into small pieces with a razor blade and transferred in a tube with 1 ml of culture medium, fixed with 1% formaldehyde for 15 min and stopped with 0.125 M glycine for 5 min. Samples were centrifuged at 4000 rpm for 2 min, washed twice and diluted in PBS. Tissues were homogenized using a pestel and suspended in 3 ml of ice-cold immunoprecipitation buffer with protease inhibitors and sonicated. ChIP was then performed as described herein.

Promoter Analysis.

Promoter analysis was performed using the MatInspector software (www.genomatix.de). A sequence of 2 kb upstream and 2 kb downstream from the transcription start site was analyzed for the presence of putative binding sites for each TFs. Primers used to amplify sequences surroundings the predicted binding sites were designed using the Primer3 software (http://frodo.wi.mitedu/cgibin/primer3/primer3_www.cgi; herein incorporated by reference in its entirety).

Quantitative RT—PCR and Immunohistochemistry.

RNA was prepared with RiboPure kit (Ambion) and subsequently used for first strand cDNA synthesis using random primers and SuperScriptll Reverse Transcriptase (Invitrogen). Real-time PCR was performed using iTaq SYBR Green from Biorad. For mNSC subclones, gene expression was normalized to GAPDH. For human GBM cell lines and GBM-derived BTSCs 18S ribosomal RNA was used.

Immunohistochemistry was performed as previously described (A41; herein incorporated by reference in its entirety). Briefly, tumors from patients with newly diagnosed glioblastoma (none of which were included in the original microarray analyses) were collected from the archival collection of the MD Anderson Pathology department. Following sectioning and deparaffinization, tumor samples were subject to antigen retrieval and incubated overnight at 4° C. with the primary antibody. The primary antibodies and dilutions were anti-YKL-40 (rabbit polyclonal, Quidel, 1:750), anti C/EBPβ, (rabbit polyclonal, Santa Cruz, 1:250) and anti-p-STAT3 (rabbit monoclonal, Cell Signaling 1:25). Scoring for YKL-40 and was based on a 3-tiered system, where 0 was <5% of tumor cells positive, 1 was 5-30% positivity and 2 was >30% of tumor cells positive. Scores of 1 and 2 were later collapsed into a single value for display purposes on Kaplan-Meier curves. Associations between C/EBPβ/Stat3 and YKL-40 were assessed using the Fisher exact test (FET). Associations between C/EBPβ/Stat3 and patients survival were assessed using the log-rank (Mantel-Cox) test of equality of survival distributions.

Microarray Analysis.

RNA was prepared with RiboPure kit (Ambion) and assessed for quality with an Agilent 2100 Bioanalyzer. Cy₃ labeled cRNA was prepared with Agilent low RNA input linear amplification kit according to the manufacturer's instructions, and hybridized to an Agilent 8×15K one-color customized array. The array was designed with E-array software 4.0 (Agilent, Palo Alto, Calif.) and included 14,851 probe sets corresponding to 2,945 mouse and 3,363 human genes. For the analysis, each array was normalized to its 75% quantile so that gene expression profiles can be compared across samples.

Gene Set Enrichment Analysis (GSEA).

To test whether specific gene signatures were globally differentially regulated, we used the Gene Set Enrichment analysis method (A31; herein incorporated by reference in its entirety). In this method, the Kolmogorov-Smirnoff test is used to determine whether two gene lists are statistically correlated. In this case, one list includes genes on the microarray expression profile dataset, ranked by their differential expression statistics across two conditions (e.g. ectopically expressed Stat3C/C/EBPβ vs. control), from most over- to most underexpressed. The other list contains non-ranked genes in a specific signature (e.g. mesenchymal). This is very useful to detect, for instance, situations where signature genes can be differentially expressed as a whole, even though the fold-change can be small for each gene in isolation. In this case, a gene-by-gene test, such as a T-test, cannot reveal statistical significance. The algorithm was set to implement weighted scoring scheme and the enrichment score significance was assessed by 1000 permutation tests.

Migration and Invasion Assays.

For the wound assay testing migration, mNSCs were plated in 60 mm dishes and grown until 95% confluence. To initiate the experiment, a scratch of approximately 400 μm was made with a P1000 pipet tip and images were taken every 24 h over the course of 4 days with an inverted microscope. In the PDGF experiment, the cells were incubated for 24 h with 20 μg/ml PDGF-BB (R&D systems) before making the scratch.

For the Matrigel invasion assay, mNSCs (1×10⁴) were added to the top of the chamber of a 24 well BioCoat Matrigel Invasion Chambers (BD) in 500 μl volume of serum free DMEM. The lower compartment of the chamber was filled with DMEM containing either 0.5% horse serum or 20 μg/ml PDGF-BB (R&D systems) as chemoattractants. After incubation for 24 h, invading cells were fixed, stained and counted according to the manufacturer's instructions. For SNB19 transduced with shRNA expressing lentivirus, 1.5×10⁴ cells were plated in the top of the chamber. The lower compartment contained 5% FBS.

Lentivirus Production and Infection.

Lentiviral expression vectors carrying shRNAs (short hairpin RNAs) specific for C/EBPβ and Stat3 were purchased from Sigma and virus stocks were prepared as recommended by the supplier. The C/EBPβ specific shRNA (shC/EBPβ) has the following sequence: 5′-CCGGCATCGACTACAAACGGAACTT CTCGAGAAGTTCCGTTTGTAGTCGATGTTTTTG-3′ (SEQ ID NO: 3). The Stat3-specific shRNA (shStat3) has the following sequence: 5′-CCGGCCTGAGTTGAATTATCAGCTTCT CGAGAAGCTGATAATTCAACTCAGGTTTTTG-3′ (SEQ ID NO: 4). To generate lentiviral particles, the lentiviral plasmids were co-transfected along with helper plasmids into human embryonic kidney 293T cells. Each shRNA expression plasmid (5 μg) was mixed with pCMVdR8.91 (2.5 μg) and pCMV-MD2.G (1 μg) vectors and transfected into human embryonic kidney 293T cells using the Fugene 6 reagent (Roche). Media from these cultures were collected after 24 h, centrifuged 10 min at 2500 rpm, passed through a 0.45-μm filter and used as source for lentiviral shRNAs. A second virus collection was performed 48 h after transfection.

To knockdown Stat3 and C/EBPβ, SNB19 (1×10⁵) were plated in 6 well culture plates and incubated for 24 h. Cells were transduced with Stat3 and C/EBPβ sh-RNA or non target control shRNA lentiviral particles. After overnight incubation, fresh culture media were exchanged, and the transduced cells were cultured in a CO₂ incubator for 5 days.

To infect GBM-derived BTSCs, lentiviral stocks were prepared as follows. Briefly, 293T cells were transfected as before with shRNA expression plasmids or non target control and supernatant collected after 24 h, centrifuged 10 min at 2500 rpm and passed through a 0.45-μm filter. The lentiviral particles were then ultracentrifuged for 1.5 h at 25,000 rpm with a SW28 rotor and diluted in 100 μl PBS1% BSA. The lentiviral titer was determined after transfection of Rat1 cells with serial dilution of the virus. GBM-derived BTSCs were plated as neurospheres in 24 well plates at 1×10⁴ cells/well and infected with shRNA expressing lentiviral stock at a multiplicity of infection (MOI) of 25. After 6 h 500 μl of fresh neurobasal medium was added. Cells were harvested after 5 days and subjected to gene expression analysis by qRT-PCR and microarray gene expression profiles.

Tumor Growth in Nude Mice and Immunohistochemistry.

6 weeks BALBc/nude mice were injected subcutaneously with C17.2 neural stem cell transduced with empty vector (bottom flank, left) or expressing Stat3C plus C/EBPβ (bottom flank, right). Four mice were injected with 2.5×10⁶ and four mice were injected with 5×10⁶ cells in 200 μA PBS/Matrigel. Mice were sacrificed after 10 (5×10⁶) or 13 weeks (2.5×10⁶) after the injection. Tumors were removed, fixed in formalin overnight and processed for the analysis of tumor histology and immunohistochemistry. Tumor sections were subjected to deparaffinization, followed by antigen retrieval and incubated overnight at 4 degrees (Nestin, CD31, FGFR-1 and OSMR) or 1 h at room temperature (Ki67) with the primary antibody. Primary antibodies and dilutions were Nestin (mouse monoclonal, BD, 1:150), CD31, (mouse monoclonal, BD, 1:100), Ki67 (rabbit polyclonal, Novocastra laboratories, 1:1000), FGFR1 (rabbit polyclonal, Abgent, 1:100), and OSMR (goat polyclonal, R&D, 1:50).

Results

Computational Identification of the Transcriptional Regulation Module Driving the Mesenchymal Signature of High-Grade Glioma.

ARACNe (Algorithm for the Reconstruction of Accurate Cellular Networks) was used to compute a comprehensive, genome-wide repertoire of regulatory interactions between any TF and the 102 genes in the MGES+ signature of high grade glioma. TFs were identified based on their Gene Ontology annotation (A16; herein incorporated by reference in its entirety) and only genes represented in the microarray expression profile data were considered in the analysis.

ARACNe is an information theoretic approach for the reverse engineering of transcriptional interactions from large sets of microarray expression profiles. This algorithm was able to identify validated targets of the MYC and NOTCH1 TFs in B and T cells (A11, A17; each herein incorporated by reference in its entirety). Here ARACNe was adapted towards a far more challenging goal, namely the unbiased identification of TFs associated with a given gene expression signature (MGES of human high grade glioma). The dataset used in this analysis included 176 grade III (anaplastic astocytoma) and grade IV (glioblastoma multiforme, GBM) samples (A1, A18, A19; each herein incorporated by reference in its entirety). These samples were previously classified into three molecular signature groups—proneural, proliferative, and mesenchymal—based on the identification of coordinated expression of specific gene sets by unsupervised cluster analysis1.

The Fisher Exact Test (FET) was then used to determine whether the ARACNe inferred targets of a TF overlaps with the MGES genes in a statistically significant way, thus indicating specificity in the regulation of the MGES+. From a list of 1018 TFs, a subset of 55 MGES+ specific regulators was inferred, at a false discovery rate (FDR) smaller than 5%. This suggests that relatively few TFs synergistically control the MGES+ signature, as indicated from a combinatorial, scale-free regulation model (hubs). Remarkably, the six most statistically significant TFs emerging from this analysis (Stat3, C/EBPβ/δ, bHLH-B2, Runx1, FosL2, and ZNF238) collectively control >74% of the MGES genes (FIG. 1). Clearly, this is a lower bound because ARACNe has a very low false positive rate but a relatively high false negative rate. Thus, many targets will be missed by the analysis.

Consistent with their previously reported activity (A20, A21; each herein incorporated by reference in its entirety), correlation analysis reveals that five are activators (Stat3, C/EBPβ/δ, bHLH-B2, Runx1, and FosL2) and one is a repressor (ZNF238) of the MGES+ genes. This can further indicate their potential as oncogenes or tumor suppressors, respectively. Since both C/EBPβ and C/EBPδ were among the top TF hubs and are known to form stoichiometric homo and heterodimers with identical DNA binding specificity and redundant transcriptional activity (A22), the term C/EBP is used generically to indicate the TF complex. The interactions inferred for each TF show statistically significant overlap, indicating that the six TFs are involved in combinatorial regulation of the MGES targets. This biochemically validated finding suggests a hierarchical, combinatorial control mechanism that provides both redundancy and fine-grain control of the mesenchymal signature of brain tumor cells by a handful of TFs.

Computational Validation of the Mesenchymal TFs Network as Regulator of the MGES.

A stepwise linear regression (SLR) method was then used to infer a simple quantitative transcriptional regulation model (i.e. a regulatory program) for the MGES+ genes. In this model, the log-expression of each target gene is approximated by a linear combination of the log-expression of a small set of TFs using linear regression (A23, A24; each herein incorporated by reference in its entirety). This allows a convenient linear representation of multiplicative interactions between TF activities (combinatorial regulation). TFs are added one at the time to the model, by choosing the one that produces the greatest reduction in the relative error on the predicted vs. observed expression, until the reduction is no longer statistically significant. TFs were then looked for that were used to model the largest number of MGES genes (see Methods). The top six TFs inferred by the FET analysis on ARACNe targets were also among the top eight inferred by SLR. Among them, the three TFs with the highest average value of their linear coupling coefficient were C/EBP (α=0.42), bHLH-B2 (α=0.41), and Stat3 (α=0.40), indicating their potential role as master regulators of the MGES genes with the next strongest TF, ZNF238, showing a negative coefficient (α=−0.34).

Biochemical Validation of TF Binding Sites.

To further validate the inferred MGES regulation network, each TF was tested for its ability to bind to the promoter region (proximal regulatory DNA) of its predicted mesenchymal targets. The target promoters were first analyzed in silico to identify putative binding sites (see Methods). ChIP assays were then performed near predicted sites in the human glioma cell line SNB75 to validate targets of Stat3, bHLH-B2, C/EBPβ and FosL2, for which appropriate reagents were available. On average, about 80% of the tested genomic regions were immunoprecipitated by specific antibodies for these TFs but not control antibodies (FIG. 3). Given that binding can occur via co-factor or outside of the selected region, this provides a conservative lowerbound of the number of actual mesenchymal targets bound by these TFs. One can conclude that ARACNe accurately recapitulates the transcriptional activity of Stat3, bHLH-B2, C/EBPβ and FosL2 on the MGES genes in malignant glioma.

Mesenchymal TFs from Malignant Glioma Form a Highly Connected and Hierarchically Organized Module.

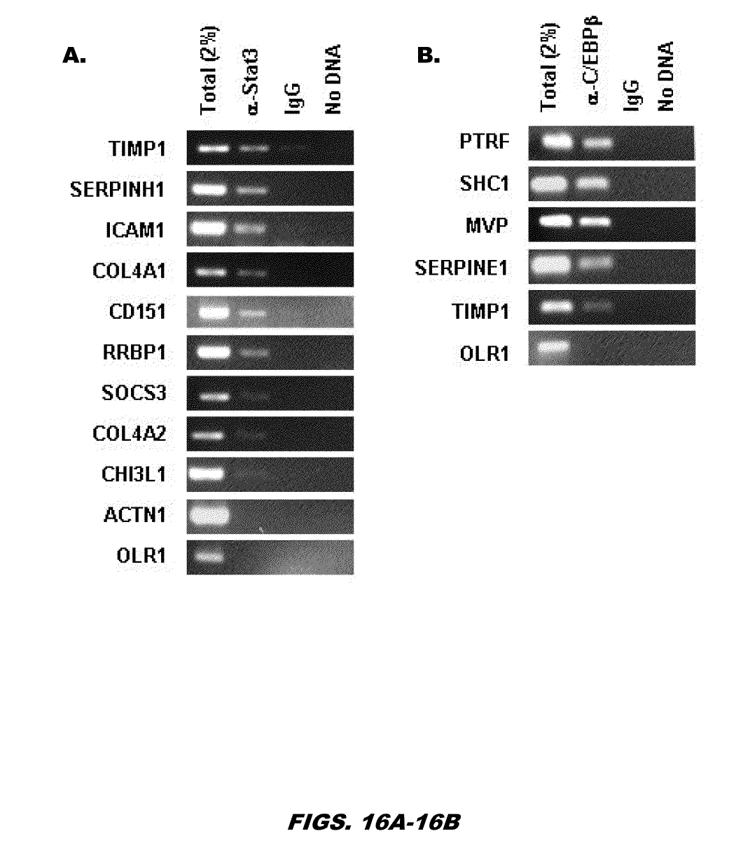

ChIP assays revealed that Stat3 and C/EBP occupy their own promoter and are thus involved in autoregulatory (AR) loops (FIG. 4A, 4B). Additionally, Stat3 occupies the FosL2 and Runx1 promoters; C/EBPβ occupies those of Stat3, FosL2, bHLH-B2, C/EBPβ, and C/EBPδ (the latter two confirm the redundant autoregulatory activity of the two C/EBP subunits, FIG. 3b) (A22, A25; each herein incorporated by reference in its entirety); FosL2 occupies those of Runx1 and bHLH-B2 (FIG. 4C); finally bHLH-B2 occupies only that of Runx1 (FIG. 4D). The MGES+ control topology that emerges from this promoter occupancy analysis is remarkably modular (high number of intra-module interactions) and displays a clearly hierarchical structure (FIG. 4E). At the very top of this hierarchical control structure, we find Stat3 and C/EBP, which are also involved in AR and form feed-forward (FF) loops with a large fraction of the MGES genes. FF loops involving only positive regulation have been shown to filter short input transient signals and thus help make such a network topology less sensitive to short, random fluctuations (A26; herein incorporated by reference in its entirety). Whether the interactions between these two TFs and the promoters of their mesenchymal targets is conserved in tumor tissues was then tested. Experimental conditions were developed to perform Stat3 and C/EBPβ ChIP assays in two human GBM samples in the mesenchymal signature group. The experiments confirmed that, also in this in vivo context, Stat3 and C/EBPβ bind to the MGES targets predicted by the computational algorithms (FIGS. 16A-16B). Taken together, these findings suggest that the six inferred TFs form a hierarchical regulatory module and that Stat3 and C/EBP can operate as master regulators of the mesenchymal signature of malignant gliomas.

Combined Expression of C/EBPβ and Stat3 Prevents Neuronal Differentiation and Reprograms Neural Stem Cells Towards the Mesenchymal Lineage.

Without being bound by theory, Neural stem cells (NSCs) are the cell of origin for malignant gliomas in the mesenchymal subgroup (A1; herein incorporated by reference in its entirety). However, whether mesenchymal transformation in glial tumors recapitulates a normal albeit rare cell fate determination event intrinsic to NSCs remains unknown (A2, A3, A9; each herein incorporated by reference in its entirety). Whether combined expression of Stat3 and C/EBPβ in NSCs is sufficient to initiate mesenchymal gene expression and to trigger the mesenchymal properties that characterize high-grade gliomas was next considered. To do this, an early passage of the stable, clonal population of mouse NSCs known as C17.2 was used. The enhanced, yet constitutively self-regulated expression of sternness genes permits these cells to be efficiently grown as undifferentiated monolayers in sufficiently large, homogeneous and viable quantities to ensure reproducible patterns of self-renewal and differentiation without ever behaving in a tumorigenic fashion in vitro or in vivo (A27-29; each herein incorporated by reference in its entirety).

Following ectopic expression of C/EBPβ and a constitutively active form of Stat3 (Stat3C) (A30; herein incorporated by reference in its entirety) in NSCs, we observed dramatic morphologic changes, consistent with loss of ability to differentiate along the neuronal lineage (FIG. 5A). Parental and vectortransfected NSCs have the classical spindle-shaped morphology that is associated with the neural stem/progenitor cell phenotype. When grown in the absence of mitogens, these cells display efficient neuronal differentiation characterized by formation of a neuritic network (FIG. 5A, top-right panel). Conversely, expression of C/EBPβ and Stat3C leads to cellular flattening and manifestation of a fibroblast-like morphology. Remarkably, depletion of mitogens resulted in additional flattening with complete loss of every neuronal trait (FIG. 5A, bottom-right panel). These results indicate that expression of C/EBPβ and Stat3C efficiently suppresses differentiation along the neuronal lineage and induces established mesenchymal features.

Next, whether C/EBPβ and Stat3C induce expression of the MGES+ genes in vivo was considered. To do this, mRNA was extracted from duplicate samples of two independent C/EBPβ/Stat3C expressing and control clones of NSCs and hybridized custom expression arrays (Agilent Technologies), containing probes for 6,308 glioma-specific mouse and human genes. The Gene Set Enrichment Analysis method (GSEA, (A31; herein incorporated by reference in its entirety)) was used to test the enrichment of the mesenchymal, proliferative and'proneural signatures (A1; herein incorporated by reference in its entirety) among differentially expressed genes in C/EBPβ/Stat3C expressing versus control cells. The algorithm was set to implement weighted scoring scheme and the enrichment score significance is assessed by 1,000 permutation tests to compute the enrichment p-value. The analysis demonstrated that the global mesenchymal and proliferative signatures are both highly enriched in genes that are overexpressed in C/EBPβ/Stat3C-expressing NSCs. Conversely, the proneural signature is enriched in genes that are underexpressed in these cells (FIG. 5B). Consistent with these findings, several mesenchymal-specific gene categories are highly enriched in C/EBPβ/Stat3C expressing NSCs.

Quantitative RT-PCR (qRT-PCR) of the microarray results was also validated for a subset of Stat3 and C/EBPβ targets. Interestingly, the genes coding for the receptors of the growth factors PDGF, EGF and bFGF were among the most upregulated genes in NSCs expressing Stat3C and C/EBPβ. Outputs from these growth factors provide essential signals for proliferation and invasion of glial tumor cells and are able to revert mature neural cells into pluripotent stem-like cells, an effect that can contribute to the mesenchymal transformation of NSCs (A32, A33; each herein incorporated by reference in its entirety). Other genes markedly overexpressed in C/EBPβ/Stat3C expressing NSCs are those coding for the morphogenetic proteins BMP4 and BMP6, two crucial inducers of tumor invasion and angiogenesis (A34, A35; each herein incorporated by reference in its entirety). Thus, Stat3 and C/EBPβ are sufficient to induce reprogramming of neuralstem cells towards an aberrant mesenchymal lineage.

Neural Stem Cells Expressing Stat3 and C/EBPβ Acquire the Hallmarks of Mesenchymal Aggressiveness and Tumorigenic Capability In Vitro and In Vivo.

Whether activation of the MGES by Stat3 and C/EBPβ is sufficient to transform NSCs into cells that can efficiently migrate and invade, two properties invariably associated with MGES+ in high grade glioma (A1, A2; each herein incorporated by reference in its entirety) was considered. The first assay used to address this question (“wound assay”) evaluates the ability to migrate and fill a scratch introduced in cultures of adherent cells (FIG. 5C). The second (“Matrigel invasion assay”) tests how cells invade a Boyden chamber filter coated with a physiologic mixture of extracellular matrix components and concentrate the lower side of the filter (FIG. 5D). When the two assays were performed on C/EBPβ/Stat3C-expressing and control NSCs clones, we found that the expression of the two TFs robustly promoted migration and invasion through the extracellular matrix (FIGS. 5C-5D). The effects of C/EBPβ and Stat3C on migration and invasion of NSCs were similar in the absence of mitogens or in the presence of PDGF (FIG. 5D). Conversely, ectopic bHLHB2 was irrelevant for the MGES and phenotypic behavior of Stat3C-C/EBPβ-expressing NSCs.

To ask whether Stat3 and C/EBPβ confer tumorigenic potential to neural stem cells in vivo, sub-cutaneous heterotopic transplantation of C17.2-Stat3C/C/EBPβ (and empty vector as control) was used. Male, six-week old BALB/nude mice (a total of eight animals in two separate experiments) were injected subcutaneously with 2.5×10⁶ and 5×10⁶ C17.2-Stat3C/C/EBPβ cells (right flank) or C17.2-Vector (left flank) in PBS-Matrigel. C17.2-Stat3C/C/EBPβ cells developed fast-growing tumors with high efficiency (4 out of 4 mice in the group injected with 5×10⁶ cells and 3 out of 4 mice in the group injected with 2.5×10⁶ cells), whereas neural stem cells transduced with empty vector never formed tumors (FIG. 6A). Histological analysis demonstrated that the tumors resembled human high grade glioma, exhibited large areas of polymorphic cells, had tendency to form pseudopalisades with central necrosis and although injected in the flank, a low angiogenic site, displayed vascular proliferation, as confirmed by immunostaining for the endothelial marker CD31 (FIGS. 6B-6C). Proliferation in the tumors was very high as determined by reactivity for Ki67. In line with the presence of stem-like cells, human GBM regularly exhibit expression of primitive markers. Corroborating this, it was found that the tumors stained positive for the progenitor marker nestin (FIG. 6C). Finally, positive immunostaining for the mesenchymal signature proteins OSMR and the FGF receptor-1 (FGFR-1) indicated that oncogenic transformation of neural stem cells had occurred in the context of reprogramming towards the mesenchymal lineage (FIG. 6D). Together, these findings establish that introduction of the two master regulators of MGES in NSCs not only induces expression of the entire mesenchymal signature but is also sufficient to transduce to these cells the key phenotypic characteristics of glioma aggressiveness that have been previously associated with the signature.

Stat3 and C/EBPβ are Essential for Expression of the MGES and Aggressiveness of Human Glioma Cells and Primary Tumors.

To assess the significance of constitutive Stat3 and C/EBPβ in the glioma cells responsible for tumor growth in humans, it was sought to abolish the expression of Stat3 and C/EBPβ in GBM-derived brain tumor stem-like cells that closely mimic the genotype, gene expression and biology of their parental primary tumors (GBM-BTSCs) (A36; herein incorporated by reference in its entirety). Transduction of GBMBTSCs with specific shRNA-carrying lentiviruses efficiently silenced endogenous Stat3 and C/EBPβ (FIG. 7A). Gene expression profile analysis using GSEA showed that depletion of Stat3 and C/EBPβ in GBM-BTSCs dramatically suppressed expression of the MGES genes (FIGS. 7B-7C). Loss of Stat3 and C/EBPβ from GBM-BTSCs led to marked down-regulation of the expression of the second layer of TFs (bHLH-B2, FosL2, Runx1) associated with the glioma derived MGES (FIG. 4F). This finding validates the hierarchical nature of the mesenchymal TFs subnetwork that emerged from ChIP (FIG. 7D).

Next, the human glioma cell line SNB19 (that clusters with tumors of the mesenchymal group) was infected with the shStat3 and shC/EBPβ lentiviruses and confirmed that silencing of Stat3 and C/EBPβ depleted the mesenchymal signature even in established glioma cell lines (FIG. 7D). Furthermore, silencing of the two master TFs of MGES in SNB19 cells eliminated 80% of their ability to invade through Matrigel (FIG. 7E). As final test for the mesenchymal regulatory role of Stat3 and C/EBPβ in human glioma, immunohistochemical analysis for C/EBPβ and active, phospho-Stat3 was conducted in human tumor specimens, and expression of these TFs was compared with YKL-40 (a well-established mesenchymal protein also known as CHI3L1) (A19, A37; herein incorporated by reference in its entirety) as well as patient outcome in a collection of 62 newly diagnosed GBMs. FET showed that expression of either C/EBPβ and Stat3 were significantly correlated with YKL-40 expression (C/EBPβ, p=4.9×10⁻⁵; Stat3, p=2.2×10⁻⁴). However, the correlation was higher when double positive tumors (C/EBPβ+/Stat3+) were compared to double negatives (C/EBPβ−/Stat3−, p=2.7×10⁻⁶). Furthermore, double positive tumors were associated with markedly worse clinical outcome than tumors that were either single or double negatives (log-rank test, p=0.0002, FIG. 7F). Positivity for either of the two TFs remained predictive of negative outcome but with lower statistical strength than double positivity (C/EBPβ, p=0.0022; Stat3, p=0.0017). These results provide compelling indication that the synergistic activation of C/EBPβ and Stat3 generates mesenchymal properties and marks the worst survival group of GBM patients.

Discussion

It has been shown that expression of Stat3 and C/EBPβ is necessary and sufficient to initiate and maintain the mesenchymal signature of high-grade glioma in neural cells. Remarkably, these two genes were identified in a completely unbiased and genome-wide fashion by a computational systems biology approach. In this context, the traditional paradigm of gene expression profile based cancer research, yielding long lists of differentially expressed genes (i.e., cancer signatures), becomes just a starting point for a more detailed and rational cellular-network based analysis where the regulators of the differentially expressed signature are identified using a causal model, reflecting physical TF-DNA interactions, rather than statistical associations. This yields a repertoire of candidate transcriptional interactions that can be further interrogated using both computational and experimental techniques to determine topology, modularity, and master regulation properties. Further computational and experimental analysis revealed that among candidate TFs, Stat3 and C/EBPβ not only directly regulate their own set of transcriptional mesenchymal targets but also participate in the hierarchical regulation of several other TFs, which were in turn validated as regulators of the MGES genes.

Taken together, these results indicate that the co-expression of C/EBPβ and constitutively active Stat3 convert neural stem cells towards a mesenchymal lineage fate with coordinated induction of a MGES+. Consistently, C/EBPβ/Stat3C-expressing neural stem cells lose their ability to differentiate along the neuronal lineage and express the normal proneural signature genes. Such a finding reflects the mutually exclusive expression of the proneural and mesenchymal signatures observed in primary GBM (A1; herein incorporated by reference in its entirety) and is further indication that C/EBPβ and Stat3C are master regulator genes, capable of inducing the mesenchymal signature of high-grade glioma in neural stem cells. Without being bound by theory, the neuroepithelial to mesenchymal reprogramming induced by Stat3 and C/EBPβ TFs in neural stem cells recapitulates the epithelial to mesenchymal transition frequently described in epithelial neoplasms undergoing progression towards a more invasive and metastatic tumor type (A38; herein incorporated by reference in its entirety). Thus, an exciting implication of this work is that, by acting upstream of the mesenchymal genes, C/EBP/Stat3-mediated transcription reprograms the cell fate of neural stem cells towards an aberrant “mesenchymal” lineage. This transformation triggers the most aggressive properties of malignant brain tumors, namely invasion and neo-angiogenesis. Since expression of Stat3 and C/EBPβ is essential to maintain the mesenchymal properties of human glioma cells, they provide important clues for diagnostic and pharmacological intervention. Immunohistochemistry assays in independent GBM samples confirmed that, based on the correlation with YKL-40, Stat3 and C/EBPβ are strongly linked to the mesenchymal state and their combined expression provides an excellent prognostic biomarker for tumor aggressiveness.

In conclusion, the studies present the first evidence that computational systems biology methods can be effectively used to infer master regulator genes that choreograph the malignant transformation of a human cell. This is a general new paradigm that will be applicable to the dissection of any normal and pathologic phenotypic state.

### REFERENCES

A1. Phillips, H. S. et al. Molecular subclasses of high-grade glioma predict prognosis, delineate a pattern of disease progression, and resemble stages in neurogenesis. Cancer Cell 9, 157-173 (2006).
A2. Tso, C. L. et al. Primary glioblastomas express mesenchymal stem-like properties. Mol Cancer Res 4, 607-619 (2006).
A3. Wurmser, A. E. et al. Cell fusion-independent differentiation of neural stem cells to the endothelial lineage. Nature 430, 350-356 (2004).
A4. Ohgaki, H. & Kleihues, P. Population-based studies on incidence, survival rates, and genetic alterations in astrocytic and oligodendroglial gliomas. J Neuropathol Exp Neurol 64, 479-489 (2005).
A5. Demuth, T. & Berens, M. E. Molecular mechanisms of glioma cell migration and invasion. J Neurooncol 70, 217-228 (2004).
A6. Kargiotis, O., Rao, J. S. & Kyritsis, A. P. Mechanisms of angiogenesis in gliomas. J Neurooncol 78, 281-293 (2006).
A7. Hoelzinger, D. B., Demuth, T. & Berens, M. E. Autocrine factors that sustain glioma invasion and paracrine biology in the brain microenvironment. J Natl Cancer Inst 99, 1583-1593 (2007).
A8. Visted, T., Enger, P. O., Lund-Johansen, M. & Bjerkvig, R. Mechanisms of tumor cell invasion and angiogenesis in the central nervous system. Front Biosci 8, e289-304 (2003).
A9. Takashima, Y. et al. Neuroepithelial cells supply an initial transient wave of MSC differentiation. Cell 129, 1377-1388 (2007).
A10. Cheng, A. S. et al. Combinatorial analysis of transcription factor partners reveals recruitment of c-MYC to estrogen receptor-alpha responsive promoters. Mol Cell 21, 393-404 (2006).
A11. Basso, K. et al. Reverse engineering of regulatory networks in human B cells. Nat Genet. 37, 382-390 (2005).
A12. Margolin, A. A. et al. ARACNE: an algorithm for the reconstruction of gene regulatory networks in a mammalian cellular context. BMC Bioinformatics 7 Suppl 1, S7 (2006).
A13. Mani, K. M. et al. A Systems biology approach to prediction of oncogenes and perturbation targets in B cell lymphomas. Molecular Systems Biology in press (2007).
A14. Hanauer, D. A., Rhodes, D. R., Sinha-Kumar, C. & Chinnaiyan, A. M. Bioinformatics approaches in the study of cancer. Curr Mol Med 7, 133-141 (2007).
A15. Lander, A. D. A calculus of purpose. PLoS Biol 2, e164 (2004).
A16. Ashburner, M. et al. Gene ontology: tool for the unification of biology. The Gene Ontology Consortium. Nat. Genet. 25, 25-29 (2000).
A17. Palomero, T. et al. NOTCH1 directly regulates c-MYC and activates a feed-forward-loop transcriptional network promoting leukemic cell growth. Proc Natl Acad Sci USA 103, 18261-18266 (2006).
A18. Freije, W. A. et al. Gene expression profiling of gliomas strongly predicts survival. Cancer Res 64, 6503-6510 (2004).
A19. Nigro, J. M. et al. Integrated array-comparative genomic hybridization and expression array profiles identify clinically relevant molecular subtypes of glioblastoma. Cancer Res 65, 1678-1686 (2005).
A20. Aoki, K. et al. RP58 associates with condensed chromatin and mediates a sequence-specific transcriptional repression. J Biol Chem 273, 26698-26704 (1998).
A21. Fuks, F., Burgers, W. A., Godin, N., Kasai, M. & Kouzarides, T. Dnmt3a binds deacetylases and is recruited by a sequence-specific repressor to silence transcription. Embo J 20, 2536-2544 (2001).
A22. Ramji, D. P. & Foka, P. CCAAT/enhancer-binding proteins: structure, function and regulation. Biochem J 365, 561-575 (2002).
A23. Bussemaker, H. J., Li, H. & Siggia, E. D. Regulatory element detection using correlation with expression. Nat Genet. 27, 167-171 (2001).
A24. Tegner, J., Yeung, M. K., Hasty, J. & Collins, J. J. Reverse engineering gene networks: integrating genetic perturbations with dynamical modeling. Proc Natl Acad Sci USA 100, 5944-5949 (2003).
A25. Niehof, M., Kubicka, S., Zender, L., Manns, M. P. & Trautwein, C. Autoregulation enables different pathways to control CCAAT/enhancer binding protein beta (C/EBP beta) transcription. J Mol Biol 309, 855-868 (2001).
A26. Kalir, S., Mangan, S. & Alon, U. A coherent feed-forward loop with a SUM input function prolongs flagella expression in Escherichia coli. Mol Syst Biol 1, 2005 0006 (2005).
A27. Lee, J. P. et al. Stem cells act through multiple mechanisms to benefit mice with neurodegenerative metabolic disease. Nat Med 13, 439-447 (2007).
A28. Park, K. I. et al. Acute injury directs the migration, proliferation, and differentiation of solid organ stem cells: evidence from the effect of hypoxia-ischemia in the CNS on clonal “reporter” neural stem cells. Exp Neurol 199, 156-178 (2006).
A29. Parker, M. A. et al. Expression profile of an operationally-defined neural stem cell clone. Exp Neurol 194, 320-332 (2005).
A30. Bromberg, J. F. et al. Stat3 as an oncogene. Cell 98, 295-303 (1999).
A31. Subramanian, A. et al. Gene set enrichment analysis: a knowledge-based approach for interpreting genome-wide expression profiles. Proc Natl Acad Sci USA 102, 15545-15550 (2005).
A32. Engebraaten, 0., Bjerkvig, R., Pedersen, P. H. & Laerum, 0. D. Effects of EGF, bFGF, NGF and PDGF(bb) on cell proliferative, migratory and invasive capacities of human brain-tumour biopsies in vitro. Int J Cancer 53, 209-214 (1993).
A33. Jackson, E. L. et al. PDGFR alpha-positive B cells are neural stem cells in the adult SVZ that form glioma-like growths in response to increased PDGF signaling. Neuron 51, 187-199 (2006).
A34. Rothhammer, T., Bataille, F., Spruss, T., Eissner, G. & Bosserhoff, A. K. Functional implication of BMP4 expression on angiogenesis in malignant melanoma. Oncogene 26, 4158-4170 (2007).
A35. Rothhammer, T. et al. Bone morphogenic proteins are overexpressed in malignant melanoma and promote cell invasion and migration. Cancer Res 65, 448-456 (2005).
A36. Lee, J. et al. Tumor stem cells derived from glioblastomas cultured in bFGF and EGF more closely mirror the phenotype and genotype of primary tumors than do serum-cultured cell lines. Cancer Cell 9, 391-403 (2006).
A37. Pelloski, C. E. et al. YKL-40 expression is associated with poorer response to radiation and shorter overall survival in glioblastoma. Clin Cancer Res 11, 3326-3334 (2005).
A38. Tarin, D., Thompson, E. W. & Newgreen, D. F. The fallacy of epithelial mesenchymal transition in neoplasia. Cancer Res 65, 5996-6000; discussion 6000-5991 (2005).
A39. Margolin, A. A. et al. Reverse engineering cellular networks. Nat Protoc 1, 662-671 (2006).
A40. Frank, S. R., Schroeder, M., Fernandez, P., Taubert, S. & Amati, B. Binding of c-Myc to chromatin mediates mitogen-induced acetylation of histone H4 and gene activation. Genes Dev 15, 2069-2082 (2001).
A41. Simmons, M. L. et al. Analysis of complex relationships between age, p53, epidermal growth factor receptor, and survival in glioblastoma patients. Cancer Res 61, 1122-1128 (2001).
A42. Zeeberg, B. R. et al. GoMiner: a resource for biological interpretation of genomic and proteomic data. Genome Biol 4, R28 (2003).

### Example 8

### A Transcriptional Module Initiates and Maintains Mesenchymal Transformation in Brain Tumors

Using a combination of cellular-network reverse-engineering algorithms and experimental validation assays, a transcriptional module, including six transcription factors (TFs) that synergistically regulates the mesenchymal signature of malignant glioma was identified. This is a poorly understood molecular phenotype, never observed in normal neural tissue. It represents the hallmark of tumor aggressiveness in high-grade glioma, and its upstream regulation is so far unknown. Overall, the newly discovered transcriptional module regulates >74% of the signature genes, while two of its TFs (C/EBPβ and Stat3) display features of initiators and master regulators of mesenchymal transformation. Ectopic co-expression of C/EBPβ and Stat3 is sufficient to reprogram neural stem cells along the aberrant mesenchymal lineage, while simultaneously suppressing differentiation along the default neural lineages (neuronal and glial). Conversely, silencing the two TFs in human glioma cell lines and glioblastoma-derived tumor initiating cells leads to collapse of the mesenchymal signature with corresponding loss of tumor aggressiveness in vitro and in immunodeficient mice after intracranial injection. In human tumor samples, combined expression of C/EBPβ and Stat3 correlates with mesenchymal differentiation of primary glioma and is a predictor of poor clinical outcome. Taken together, these results reveal that activation of a small regulatory module—inferred from the accurate reconstruction of transcriptional networks—is necessary and sufficient to initiate and maintain an aberrant phenotypic state in eukaryotic cells.

High-grade gliomas (HGGs) are the most common brain tumors in humans and are essentially incurable (Ohgaki, 2005; herein incorporated by reference in its entirety). Just as the ability to metastasize identifies the highest degree of malignancy in epithelial tumors, the defining hallmarks of aggressiveness of glioblastoma multiforme (GBM) are local invasion and neo-angiogenesis {Demuth, 2004; Kargiotis, 2006; each herein incorporated by reference in its entirety}. Drivers of these phenotypic traits include intrinsic autocrine signals produced by brain tumor cells to invade the adjacent normal brain and stimulate formation of new blood vessels {Hoelzinger, 2007; herein incorporated by reference in its entirety}. It has been suggested that GBM re-engages pre-established ontogenetic motility and invasion signals that normally operate in neural stem cells (NSCs) and immature progenitors {Visted, 2003; herein incorporated by reference in its entirety}. A recently established notion postulates that neoplastic transformation in the central nervous system (CNS) converts neural stem cells into cell types manifesting a mesenchymal phenotype, a state associated with uncontrolled ability to invade and stimulate angiogenesis {Phillips, 2006; Tso, 2006; each herein incorporated by reference in its entirety}. Differentiation along the mesenchymal lineage, however, is virtually undetectable in normal neural tissue during development. Specifically, gene expression studies have established that over-expression of a “mesenchymal” gene expression signature (MGES) and loss of a proneural signature (PNGES), co-segregate with the poorest prognosis group of glioma patients {Phillips, 2006; herein incorporated by reference in its entirety}. The MGES↑PNGES↓ phenotype can thus be referred to as the mesenchymal phenotype of high-grade glioma. Without being bound by theory, drift toward the mesenchymal lineage may be exclusively an aberrant event that occurs during brain tumor progression. Without being bound by theory, glioma cells may recapitulate the rare mesenchymal plasticity of NSCs {Phillips, 2006; Takashima, 2007; Tso, 2006; Wurmser, 2004; each herein incorporated by reference in its entirety}. The molecular events that trigger activation of the MGES and suppression of the PNGES signatures, imparting a highly aggressive phenotype to glioma cells, remain unknown.

To discover transcription factors (TFs) causally linked to overexpression of MGES genes, the conventional paradigm of gene expression profile-based cancer research was inverted. Rather than asking which genes comprise the MGES, a genome-wide, glioma-specific map of transcriptional interactions was inferred and then interrogated to identify TFs controlling MGES induction in vivo. Efforts to identify TFs associated with specific cancer signatures from regulatory networks have yet to produce experimentally validated discoveries, likely because these networks are still poorly mapped, especially within specific mammalian cellular contexts {Rhodes, 2005; herein incorporated by reference in its entirety}. However, extension of reverse engineering approaches to the genome-wide inference of regulatory networks in mammalian cells have recently shown some promise {Basso, 2005, Margolin, 2006; each herein incorporated by reference in its entirety}. These methods have been further refined to identify causal, rather than associative interactions {Margolin, 2006; herein incorporated by reference in its entirety}, and have been successfully applied to the identification of dysregulated genes within developmental and tumor-related pathways {Zhao, 2009, Lim, 2009, Mani, 2007, Palomero, 2006, Taylor, 2008; each herein incorporated by reference in its entirety}. It was reasoned that the context-specific regulatory networks inferred by these algorithms may provide sufficient accuracy to allow estimating (a) the activity of TFs from that of their transcriptional targets or regulons and (b) TFs that are Master Regulators (MRs) of specific eukaryotic signatures {Hanauer, 2007; Lander, 2004; each herein incorporated by reference in its entirety} from the overlap between their regulons and the signatures. Thus, by studying the overlap between the MGES of malignant glioma and the computationally-inferred regulon of each TF, the aim was to unravel the complement of primary TFs activated and suppressed in that phenotype and, more specifically, those associated with its induction in human brain tumors.

TFs causally linked with MGES activation were first identified using the published dataset {Phillips, 2006; herein incorporated by reference in its entirety}. Next, it was discovered that the same TFs are associated with induction of a poor prognosis signature in the distinct GBM sample set from the Atlas-TCGA consortium {Network, 2008; herein incorporated by reference in its entirety}. Comprehensive computational and experimental assays converged on two of these TFs (C/EBP and Stat3) as synergistic initiators and essential MRs of the MGES of human glioma. Indeed, ectopic co-expression of the two factors in NSCs was sufficient to initiate expression of the mesenchymal set of genes, suppress proneural genes, promote mesenchymal transformation and trigger invasion. Conversely, silencing of these TFs consistently depleted GBM-derived brain tumor initiating cells (GBM-BTICs) and glioma cell lines of mesenchymal attributes and greatly impaired their ability to initiate brain tumor formation after intracranial transplantation in the mouse brain. Most notably, independent immunohistochemistry experiments in 62 human glioma specimens showed that concurrent expression of C/EBPβ and Stat3 is significantly associated to the expression of mesenchymal proteins and is an accurate predictor of the poorest outcome of glioma patients.

Computational Identification of the Transcriptional Regulation Module Driving the Mesenchymal Signature of High-Grade Glioma.

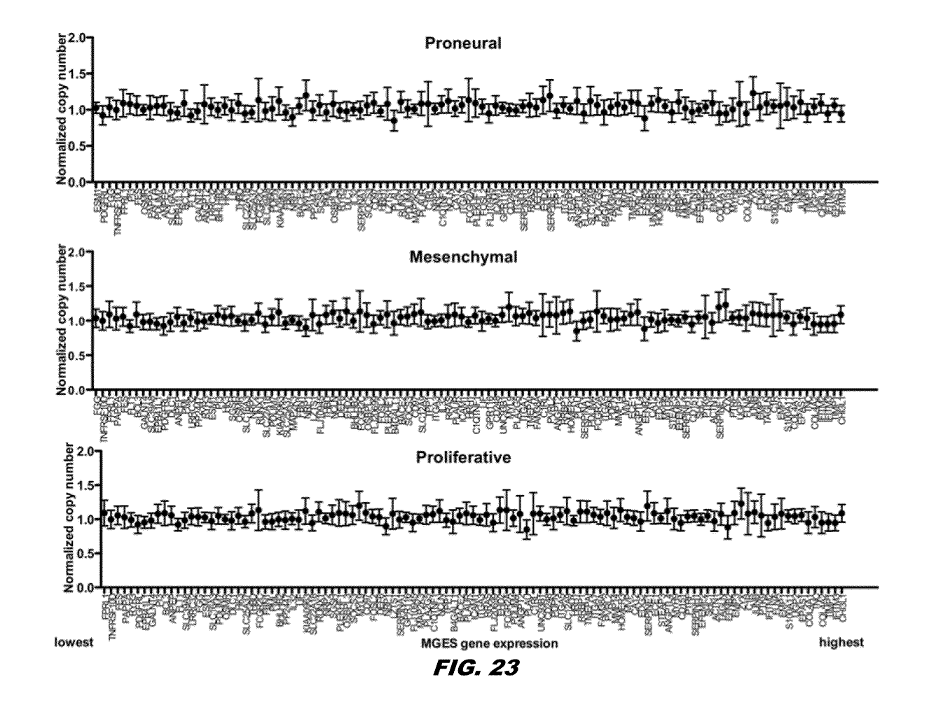

To identify the causal events that activate the MGES in HGGs, whether copy number variation alone may account for the aberrant expression of all or some of its genes was first asked. Integrated analysis of 76 HGGs for gene expression profiling and array comparative genomic hybridization (aCGH) failed to show any correlation between mean expression value and DNA copy number of MGES genes in tumors from any of the molecular subgroups (proneural, mesenchymal, and proliferative, see Methods and FIG. 23). Thus, it was sought to identify candidate MR-TFs, which may functionally activate the MGES in HGGs, using an unbiased computational approach.

The ARACNe reverse-engineering algorithm {Basso, 2005; herein incorporated by reference in its entirety} was used to assemble a genome-wide repertoire of HGGs-specific transcriptional interactions (the HGG-interactome), from 176 gene expression profiles of grade III (anaplastic astrocytoma) and grade IV (GBM) samples {Freije, 2004; Nigro, 2005; Phillips, 2006; each herein incorporated by reference in its entirety}. These specimens had been previously classified into three molecular signature groups—proneural, proliferative, and mesenchymal—based on the coordinated expression of specific gene sets by unsupervised cluster analysis {Phillips, 2006; herein incorporated by reference in its entirety} (see Table 3A-C). ARACNe is an information theoretic approach for the inference of TF-target interactions from large sets of microarray expression profiles. It previously identified targets of MYC and NOTCH 1 in B and T cells respectively, which were experimentally validated {Basso, 2005; Palomero, 2006; each herein incorporated by reference in its entirety}. It was later refined to infer directed (i.e. causal) interactions by considering only those involving at least one GO-annotated TF {Ashburner, 2000; herein incorporated by reference in its entirety} (see Methods) and by assuming that direct information transfer between mRNA species is mostly mediated by transcriptional interactions {Margolin, 2006; herein incorporated by reference in its entirety}. Thus, all interactions in the HGG-interactome, except those between two TFs (<10% of the total), are directed and thus explicitly model causality. These included 117,789 transcriptional interactions, 1,563 of which were between TFs and 102 of the 149 MGES genes {Phillips, 2006; herein incorporated by reference in its entirety} represented across all the gene expression profile data.

Next, a Master Regulator Analysis (MRA) algorithm was applied to the HGG-interactome (see Methods). The algorithm used the statistical significance of the overlap between each TF regulon (the ARACNe-inferred targets of the TF) and the MGES genes (MGES-enrichment) to infer the TFs that are more likely to regulate signature activity. Enrichment p-values were measured by Fisher Exact Test (FET). From a list of 928 TFs (Table 4), the MRA inferred 53 MGES-specific TFs, at a False Discovery Rate (FDR) <5% (Table 5A). These were ranked based on the total number of MGES targets they regulated. The top six TFs (Stat3, C/EBPβ/δ, bHLH-B2, Runx1, FosL2, and ZNF238) collectively controlled >74% of the MGES genes, suggesting that a signature core may be controlled by a relatively small number of TFs (FIG. 1). Consistent with their previously reported activity {Aoki, 1998; Fuks, 2001; each herein incorporated by reference in its entirety}, Spearman correlation analysis revealed that five of these TFs are likely activators (Stat3, C/EBPβ/δ, bHLH-B2, Runx1, and FosL2) and one is likely a repressor (ZNF238). Overlap between the regulons of the six TFs was highly significant (Table 6), suggesting coordinated and potentially synergistic regulation of the MGES. Both C/EBPβ and C/EBPδ were among the most MGES-enriched TFs. These are known to form stoichiometric homo and heterodimers with identical DNA binding specificity and redundant transcriptional activity {Ramji, 2002; herein incorporated by reference in its entirety}. One can thus use the term C/EBP to indicate the TF complex and the union of their targets as the corresponding regulon.

Similar MRA analysis of the Proneural (PNGES) and Proliferative (PROGES) signatures of HGGs was conducted (Table 7). Virtually no overlap among candidate MRs of the three signatures was detected, with the notable exception of a handful of TFs inversely associated with MGES and PNGES activation (OLIG2, for instance, activates 46 proneural and represses 12 mesenchymal genes, respectively). These results are consistent with the notion that proneural and mesenchymal genes in HGGs are mutually exclusive {Phillips, 2006; herein incorporated by reference in its entirety}. It also indicates that the reconstruction of the network topology and the application of the MRA algorithm to HGG samples are not biased towards the identification of specific TFs. Note that the impact of potential false negatives from ARACNe is considerably reduced since MRA analysis is based on enrichment criteria rather than on the identification of specific targets.

Inference of Regulatory Programs Controlling Individual MGES Genes.

Stepwise linear regression (SLR) was then used to infer simple, quantitative regulation models for each MGES gene (i.e. a regulatory program). In these models, the log-expression of each MGES gene is approximated by a linear combination of the log-expression of 53 ARACNe-inferred and 52 additional TFs, whose DNA-binding signature was enriched in MGES gene promoters (see Methods). Six TFs were in both lists, for a total of 99 TFs (Table 5B). The log-transformation allows convenient linear representation of multiplicative interactions between TF activities {Bussemaker, 2001; Tegner, 2003; each herein incorporated by reference in its entirety}. TFs were individually added to the model, each time selecting the one contributing the most significant reduction in relative expression error (predicted vs. observed), until error-reduction was no longer significant. Thus, expression of each MGES gene was defined as a function of a small number of TFs (1 to 5). Finally, TFs were ranked based on the fraction of MGES genes they regulated. Surprisingly, the top six MRA-inferred TFs were also among the eight controlling the largest number of MGES targets, based on SLR analysis (Table 8). This finding provides further support for a regulatory role of these TFs in the control of the MGES. Among them, the three TFs with the highest linear-regression coefficient values were C/EBP (α=0.40), bHLH-B2 (α=0.41), and Stat3 (α=0.40), thus establishing them as likely MGES-MR candidates. The next strongest TF, ZNF238, had a negative coefficient (α=−0.34) confirming its role as a strong MGES repressor.

Biochemical and Functional Validation of the ARACNe/MRA Regulatory Module.

It was sought to experimentally validate the TFs inferred as positive regulators of the MGES in HGGs. The first consideration was whether these TFs could bind the promoter region (proximal regulatory DNA) of their predicted MGES targets. Target promoters were first analyzed in silico to identify putative binding sites (see Methods). Chromatin Immunoprecipitation (ChIP) assays were then performed near predicted sites in a human glioma cell line to validate the ARACNe-inferred targets of four of the five TFs (C/EBPβ, Stat3, bHLH-B2, and FosL2), for which appropriate reagents were available. On average, TF-specific antibodies (but not control antibodies) immunoprecipitated with 80% of the tested genomic regions (FIG. 3). Given that binding may occur via co-factors, via non-canonical binding sites, or outside the selected region, this provides a conservative lower-bound on the number of their bound MGES targets.

Next, lentivirus-mediated shRNA silencing of the five TFs (C/EBPβ, Stat3, bHLH-B2, FosL2, and Runx1) was performed in the SNB19 human glioma cell line, followed by gene expression profiling using HT-12v3 Illumina BeadArrays in triplicate. GSEA analysis revealed: (a) that genes differentially expressed following shRNA-mediated silencing of each TF were enriched in its ARACNe-inferred regulon genes (but not in those of equivalent control TFs) (Table 9A); (b) that, consistent with predicted TF-regulon overlap, cross-enrichment among the TFs was also significant (Table 9A), suggesting that these TFs may work as a regulatory module; and (c) that genes differentially expressed following silencing of each TF were also enriched in MGES genes (Table 9B). Taken together, these results suggest that ARACNe and MRA accurately predicted the modular regulation of the MGES by these five TFs in malignant glioma.

TFs Controlling MGES in Malignant Glioma Form a Highly Connected and Hierarchically Organized Module.

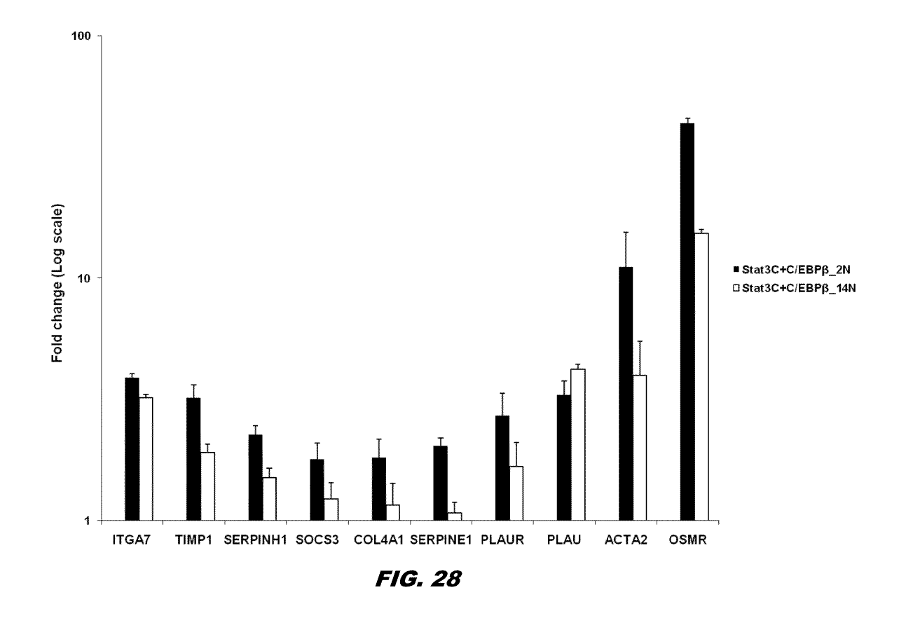

It was considered whether the inferred TFs could be organized into a regulatory module. ChIP assays revealed that C/EBPβ and Stat3 occupy their own promoter and are thus likely involved in autoregulatory (AR) loops (FIG. 4A-B). Additionally, Stat3 occupies the FosL2 and Runx1 promoters (FIG. 4A); C/EBPβ occupies those of Stat3, FosL2, bHLH-B2, C/EBPβ, and C/EBPβ, thus confirming the redundant autoregulatory activity of the two C/EBP subunits (FIG. 4B) {Niehof, 2001; Ramji, 2002; each herein incorporated by reference in its entirety}; FosL2 occupies those of Runx1 and bHLH-B2 (FIG. 4C) and bHLH-B2 occupies only the promoter of Runx1 (FIG. 4D). The MGES regulatory-control topology that emerges from promoter occupancy analysis is highly modular, with 8 of 10 possible intra-module interactions implemented (p=1.0×10⁻⁸ by FET, based on the ratio of intra- vs. inter-module interactions for equally connected TFs) and displays a clearly hierarchical structure (FIG. 4E). At the very top of this hierarchical control structure, we find C/EBP and Stat3, which are also involved in AR loops and form feed-forward (FF) loops with the largest fraction of MGES genes (43%) than any of the other TF-pairs. Accordingly, shRNA-mediated co-silencing of C/EBPβ and Stat3 in glioma cells produced >2-fold reduction of the levels of the mRNAs coding for the second layer TFs in the FF loops (bHLH-B2, FosL2, and Runx1), thus further supporting a hierarchical modular structure (FIG. 16A). Whether C/EBPβ and Stat3 bound the promoters of their MGES targets also in primary tumors was tested. Experimental conditions were developed to perform C/EBPβ and Stat3 ChIP assays in two human GBM samples belonging to the mesenchymal signature group. These assays confirmed that C/EBPβ and Stat3 bind to their inferred MGES targets also in this in vivo context (FIG. 28).

Cross-Species Integrative Analysis of Mouse and Human Cells Carrying Perturbations of C/EBPβ and Stat3.

The above results suggest that C/EBPβ and Stat3 may operate as cooperative and possibly synergistic MRs of MGES activation in malignant glioma. To functionally validate this hypothesis, gain and loss-of-function experiments were conducted for the two TFs in NSCs and human glioma cells, respectively. NSCs have been proposed as the cell of origin for malignant glioma in the mesenchymal subgroup {Phillips, 2006; herein incorporated by reference in its entirety}. Two populations of murine NSCs were infected with retroviruses expressing C/EBPβ and a constitutively active form of Stat3 (Stat3C) {Bromberg, 1999; herein incorporated by reference in its entirety}. These included an early passage of the stable, clonal population of v-myc immortalized mouse NSCs known as C17.2 {Lee, 2007; Park, 2006; Parker, 2005; each herein incorporated by reference in its entirety} as well as primary murine NSCs derived from the mouse telencephalon at embryonic day 13.5.

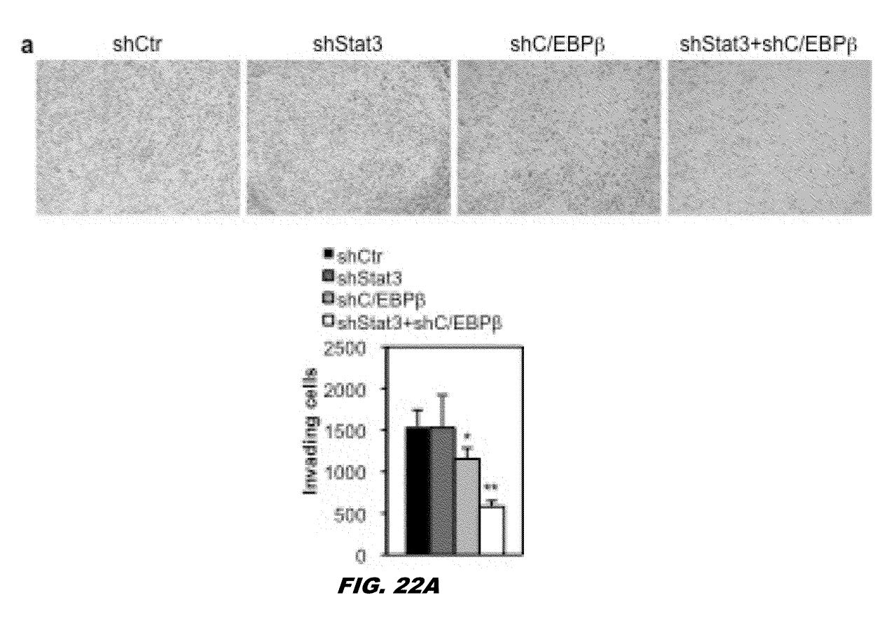

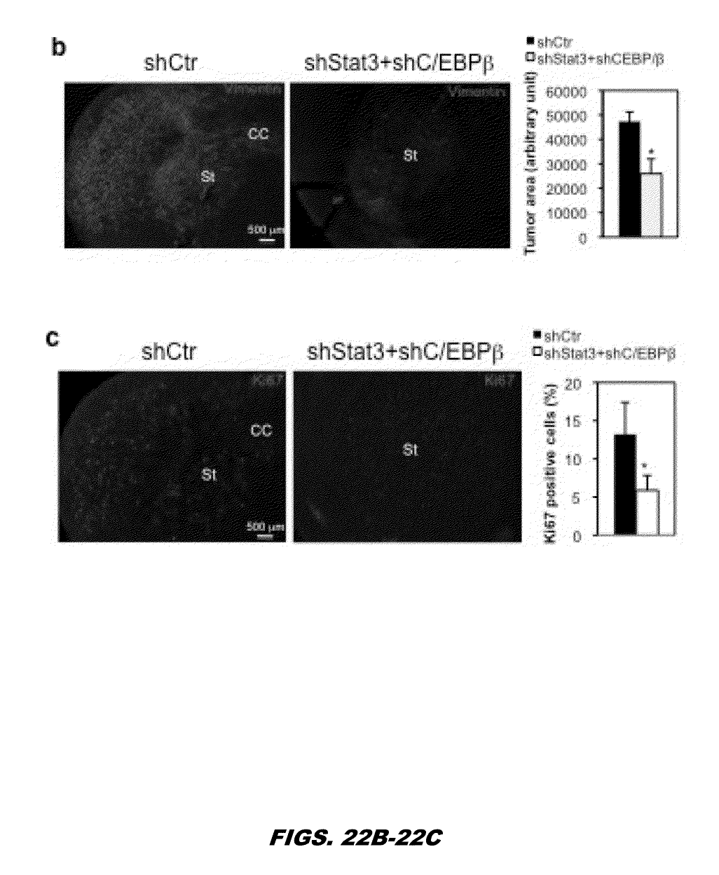

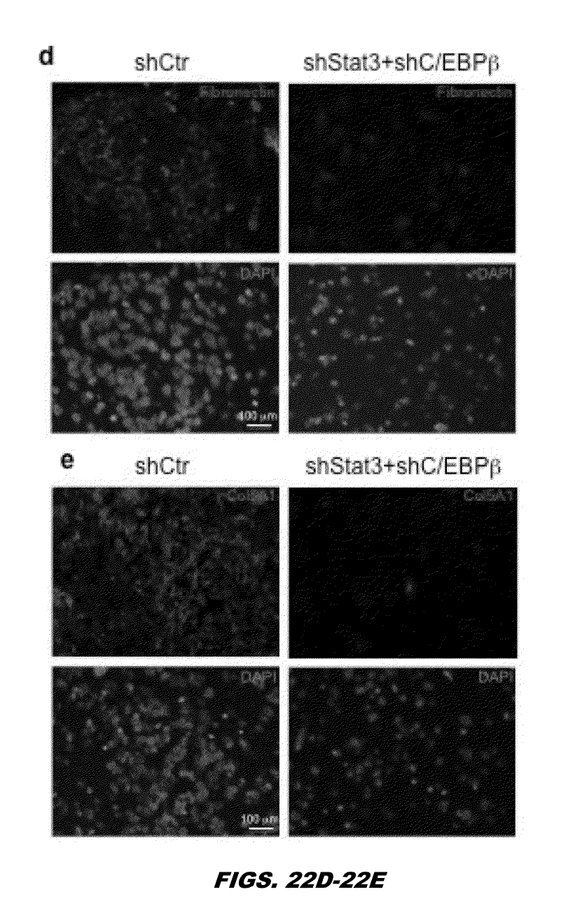

For loss-of-function experiments, lentivirus-mediated shRNA silencing of C/EBPβ and Stat3 in the human glioma cell line SNB19 and in early-passage cultures of tumor cells derived from primary GBM was performed. The latter were grown in serum-free conditions, in the presence of the growth factors bFGF and EGF. These culture conditions preserve the tumor stem cell-like features of GBM-derived cells and propel the formation of GBM-like tumors after intracranial transplantation in immunodeficient mice {Lee, 2006; herein incorporated by reference in its entirety} (GBM-derived brain tumor initiating cells, GBM-BTICs, see FIG. 22 for the analysis of their tumor-initiating capacity). At least three replicates for each condition were produced and a global dataset of 89 individual samples was generated, including 55 knockdown experiments in human glioma cells and 34 ectopic expression experiments in mouse NSCs. Gene expression profiles of human samples were produced with the HT-12v3 Illumina BeadArrays (including 24,385 human genes), while murine samples were profiled on mouse-6V2 Illumina BeadArrays (including 20,311 mouse genes). 14,857 murine genes were mapped to human orthologs, using the homologene database (http://www.ncbi.nlm.nih.gov/homologene; herein incorporated by reference in its entirety). Of the 149 genes in the MGES, 118 could be mapped to murine genes represented on the mouse-6V2 array.

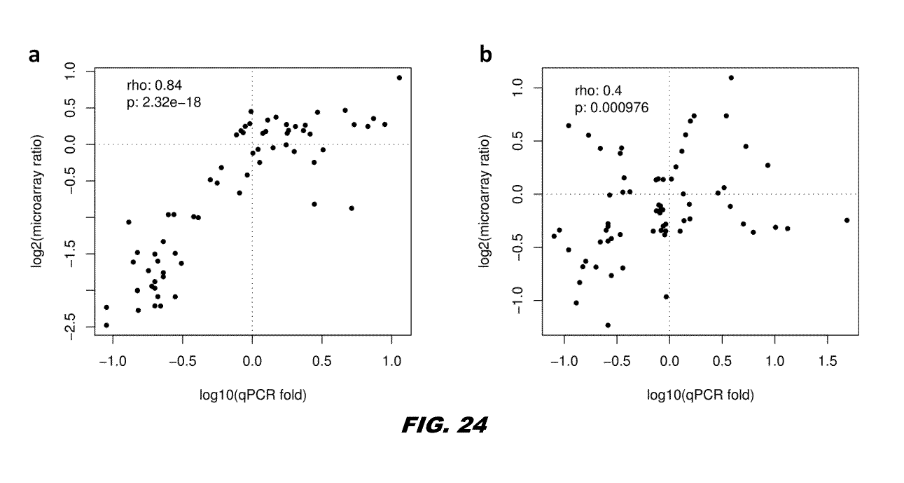

Quantitative RT-PCR (qRT-PCR) analysis performed on each sample showed that C/EBPβ and Stat3 were effectively silenced and overexpressed (Table 10). Following C/EBPβ shRNA silencing in GBM-BTICs and SNB19, C/EBPβ mRNA levels measured by qRT-PCR were significantly reduced compared to non-target control transduced cells (fold ratio=0.26, p≦0.00108, by U-test). Slightly stronger reduction was observed for Stat3 mRNA in Stat3-shRNA silenced cells (fold ratio=0.205, p≦0.00109, U-test). Reciprocal changes followed ectopic expression of the two TFs in C17.2 and NSC cells (Table 10) qRT-PCR values and microarray-based measurements were highly correlated for Stat3 but not for C/EBPβ mRNA (FIG. 24). Moreover, the Stat3C and C/EBPβ constructs used in the ectopic expression experiments in mouse NSCs lack the 3′ UTR sequence targeted by the Illumina probes. Thus, the qRT-PCR values for C/EBPβ and Stat3 were used, rather than the microarray measurements, as more accurate read-outs for their mRNA expression across the 89 samples.

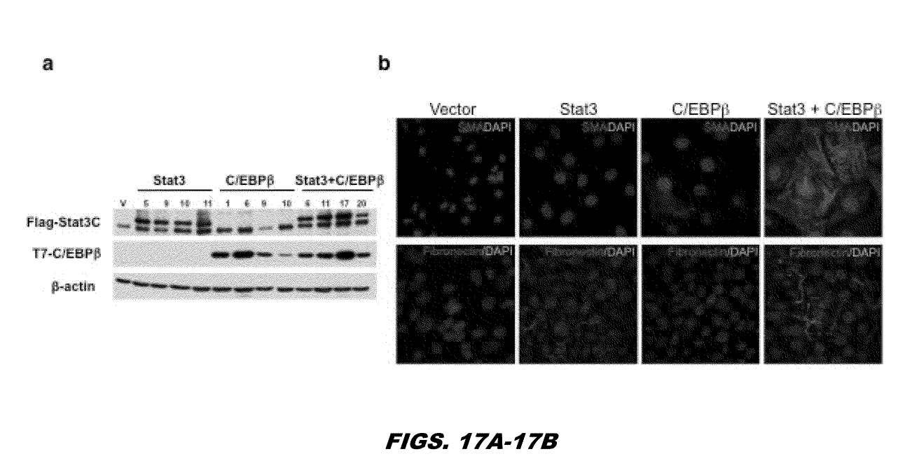

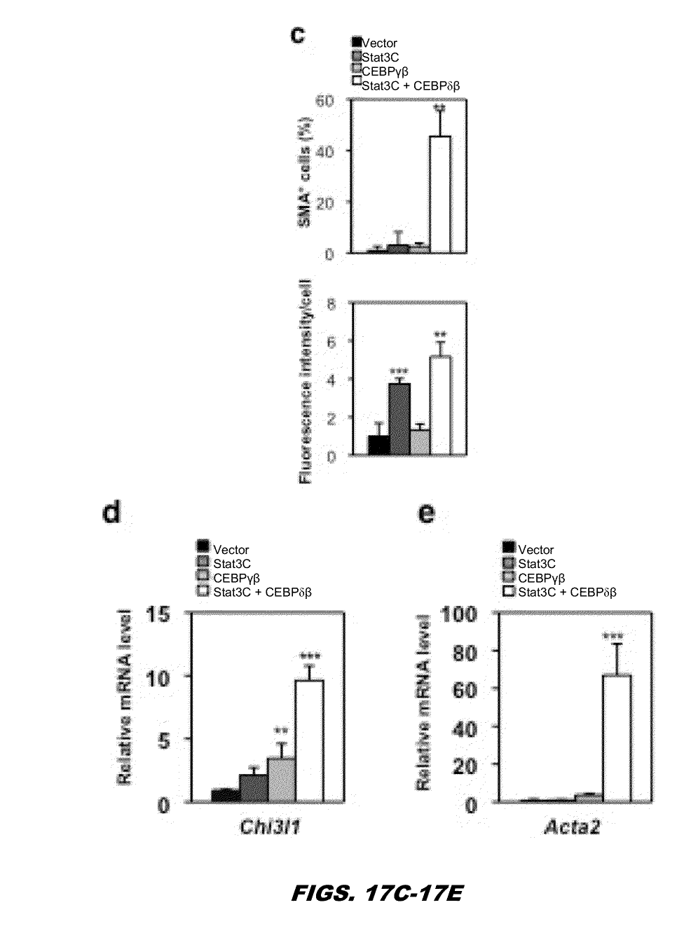

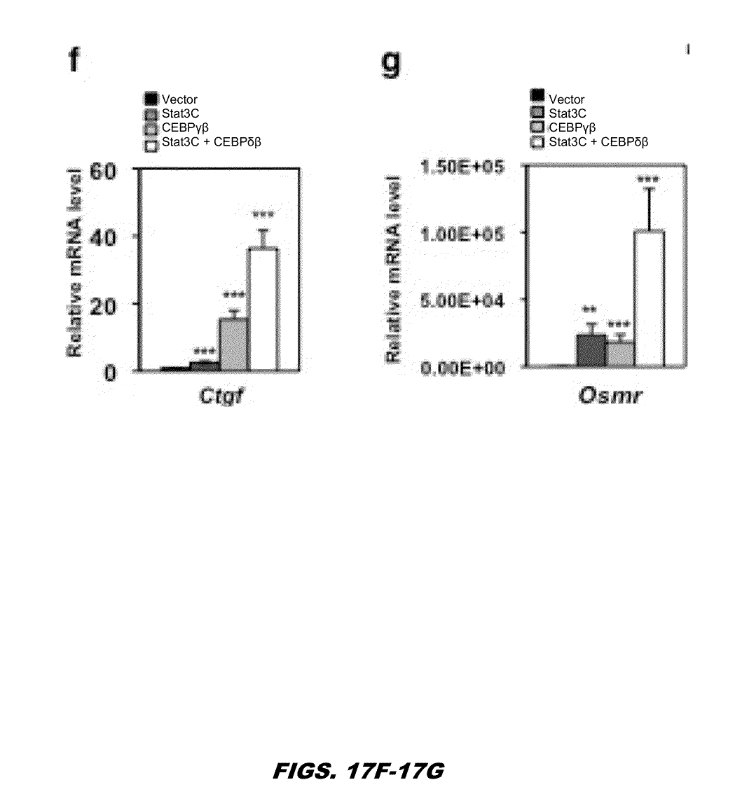

First, it was considered whether this large set of experiments demonstrated specific regulation of C/EBPβ and Stat3 ARACNe-inferred targets. GSEA analysis confirmed that genes co-expressed with the two TFs across the 89 samples were significantly enriched in their respective ARACNe-inferred regulon genes but not in those of control TFs (Table 11). More importantly, the GSEA analysis showed that perturbation of either C/EBPβ (FIG. 17A, FIG. 17D) or Stat3 (FIG. 17B, FIG. 17E) affected the MGES signature specifically (p=2.67×10⁻² and p=2.0×10⁻⁴, respectively by GSEA). Interestingly, common targets of both C/EBP and Stat3 were 8-fold more enriched in MGES genes than targets controlled individually by each TF (FIG. 17G) (p=2.25×10⁻⁵), suggesting synergistic regulation. To test whether the two TFs may be involved in synergistic MGES control, a metagene (C/EBPβ×Stat3) was created whose expression was proportional to the product of their mRNAs. The expression profile of any target regulated synergistically by the two TFs (i.e., by multiplicative rather than additive logic) should be highly correlated with such a metagene (FIG. 17C). GSEA analysis confirmed that genes ranked by Spearman correlation to the C/EBPPxStat3 metagene were significantly enriched in MGES genes (FIG. 17F). This suggests that at least a subset of the MGES follows a multiplicative (synergistic) model of regulation, while another subset may be individually regulated by C/EBPβ or Stat3 (complementarity). Taken together, these experiments support a cooperative and synergistic control of the MGES by C/EBPβ and Stat3 across a large subset of murine NSC and human glioma contexts, with MGES genes responding to both silencing and overexpression of the two TFs.

Signature and Dataset-Independent Validation of the Identification of MRs in HGG.

The MGES was originally identified as common biological attribute of a fraction of the samples associated with the poorest prognosis group of HGGs. It was sought to establish whether i) MRs inferred by the procedure would also be inferred when using an entirely independent glioma sample datasets and it) MRs identified purely on the basis of clinical outcome would overlap significantly with those inferred from analysis of the MGES signature. The MRA and SLR approaches were thus applied to the independent glioma dataset provided by the Atlas-TCGA consortium {Network, 2008; herein incorporated by reference in its entirety}. This dataset includes 77 and 21 samples associated with worst- and best-prognosis, respectively (92 samples with intermediate prognosis were not considered). Differential expression analysis identified a TCGA Worst-Prognosis Signature (TWPS), comprising 884 genes differentially expressed in the worst-prognosis samples compared to the best-prognosis ones (p≦0.05 by Student's t-test, Table 12).

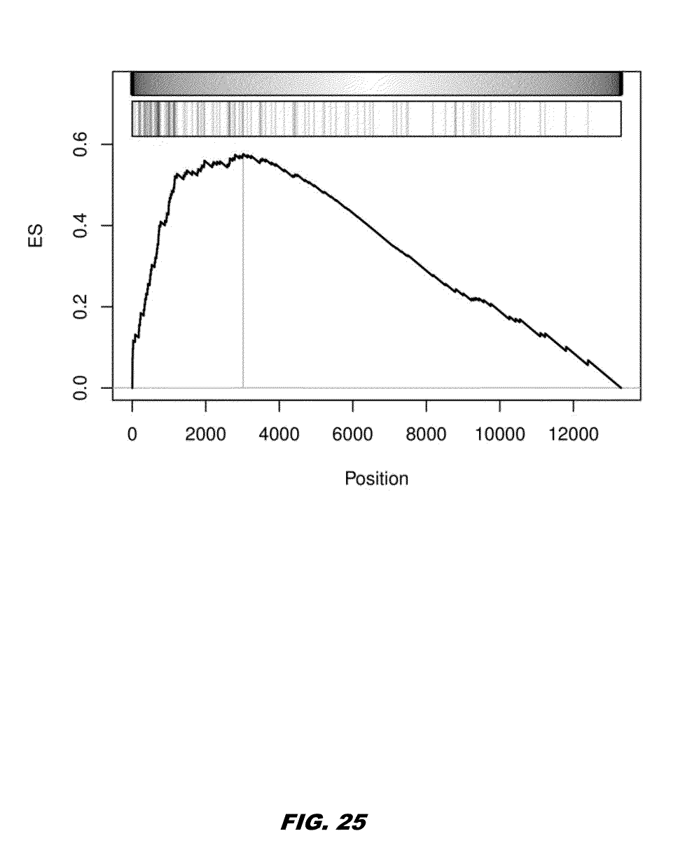

GSEA analysis confirmed that MGES genes identified in Phillips, 2006; herein incorporated by reference in its entirety were markedly enriched in the TWPS signature (p≦1.0×10⁻⁴, FIG. 25), suggesting that the poor-prognosis group in the Atlas-TCGA dataset also displays a markedly mesenchymal phenotype. However, overlap between MGES and TWPS genes was partial (22.8%), indicating that other previously unrecognized “mesenchymal” genes should be added to the MGES and/or that other biologically relevant functions may cooperate with mesenchymal transformation to produce the poor-prognosis cluster of HGGs. Nonetheless, five of the 10 most significant MRs identified by MRA analysis from the original dataset, including 4 out of 5 of our positive MGES modulators (C/EBPβ, C/EBPδ, Stat3, bHLH-B2, and FosL2), were also found among the 10 most significant TFs identified by TWPS-based analysis of the Atlas-TCGA dataset. Specifically, C/EBP was inferred as the most significant TF (C/EBPδ and C/EBPβ were 3^{rd} and 10^{th}, respectively), while Stat3 was in 7^{th} position. Additionally, among the top 10 TFs, C/EBPβ and C/EBPδ had respectively the first and second best linear-regression coefficient by SLR analysis (Table 13). These results suggest significant robustness of the approach both to dataset and signature selection. Furthermore, these findings suggest that the MGES and a more comprehensive signature broadly associated with the poorest-prognosis are regulated by the same TFs, including C/EBP and Stat3 among the top-ranking ones. Recently, there have been several unsuccessful attempts to identify common expression signatures from different sample sets representative of the same phenotype {Ein-Dor, 2005; herein incorporated by reference in its entirety}. These findings indicate that MRs of mammalian phenotype signatures may be significantly more conserved than their specific genes.

Concurrent Expression of Active C/EBPβ and Stat3 Reprograms NSCs Toward the Mesenchymal Lineage.

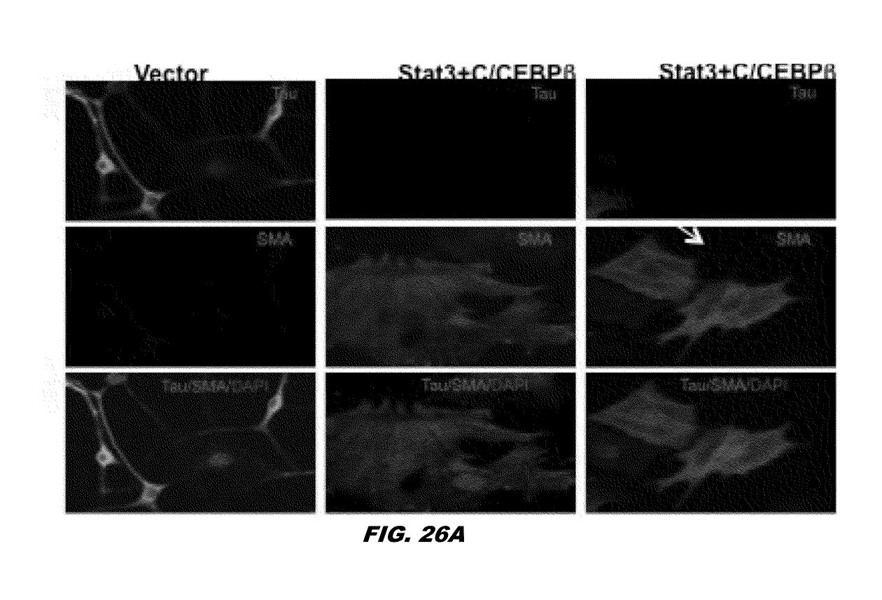

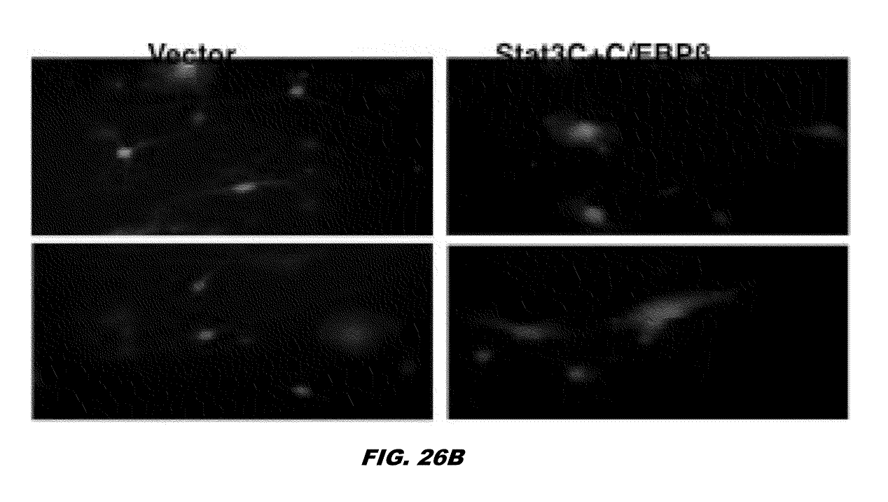

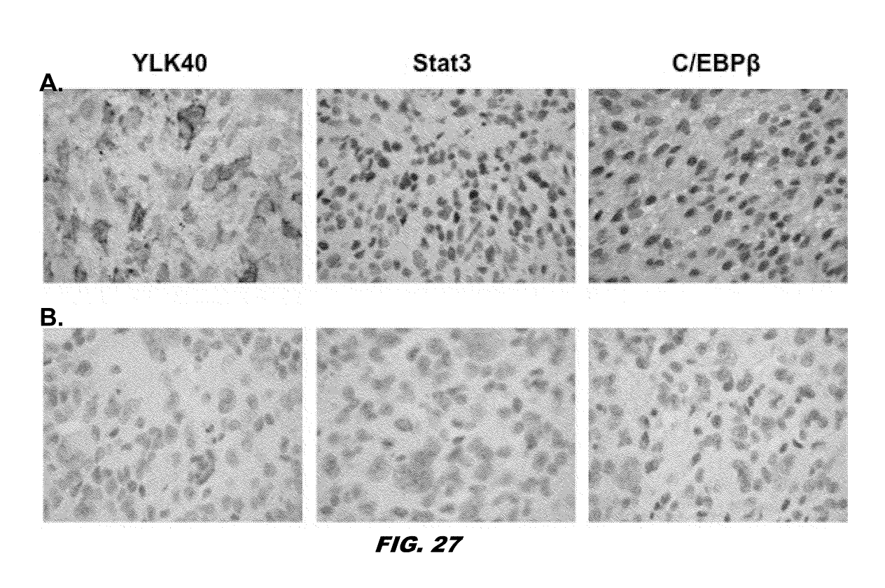

Having shown that manipulation of C/EBPβ and Stat3 results in corresponding changes in the MGES, the next question was whether these effects are associated with phenotypic changes. First, it was considered whether combined and/or individual expression of Stat3C and C/EBPβ in NSCs is sufficient to trigger the mesenchymal phenotypic properties that characterize high-grade gliomas. Ectopic expression of C/EBPβ and Stat3C in C17.2 NSCs induced dramatic morphologic changes, consistent with loss of ability to differentiate along the default neuronal lineage (FIG. 5A, FIG. 26A). Parental and vector-transfected NSCs have the classical spindle-shaped morphology that is associated with the neural stem/progenitor cell phenotype. When grown in the absence of mitogens, these cells display efficient neuronal differentiation characterized by extensive formation of a neuritic network. Conversely, expression of Stat3C and C/EBPβ led to cellular flattening and manifestation of a fibroblast-like morphology (FIG. 26A).

Ectopic expression of C/EBPβ and Stat3C cooperatively induced the expression of mesenchymal markers in NSCs. This was shown with immunofluorescence staining for SMA and fibronectin in C17.2 expressing the indicated TFs. SMA positive cells were quantified. For fibronectin immunostaining, the intensity of fluorescence was quantified. QRT-PCR analysis of mesenchymal targets in C17.2 expressing the indicated TFs or transduced with the empty vector was also carried out. Gene expression was normalized to the expression of 18S ribosomal RNA.

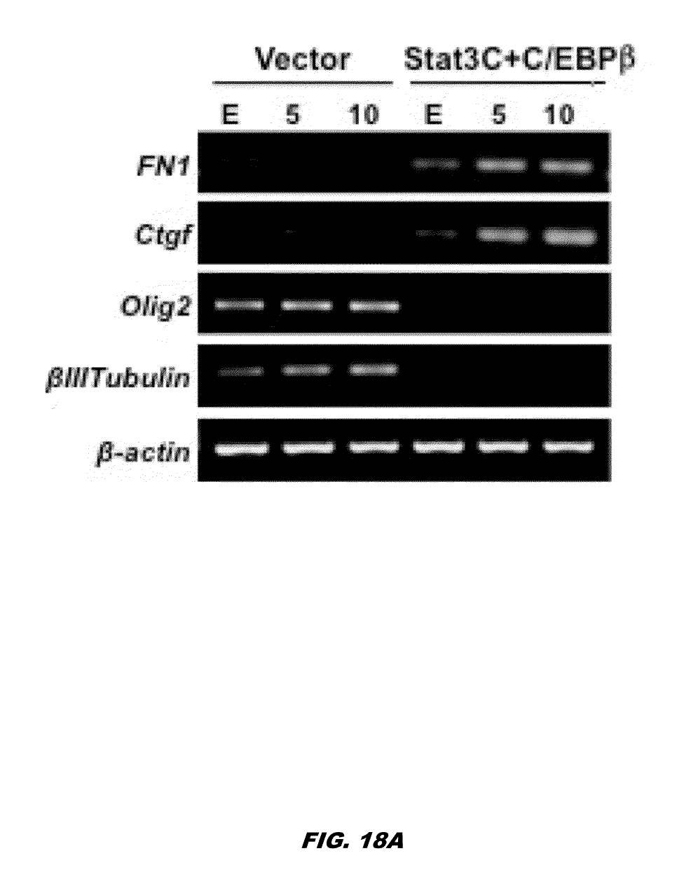

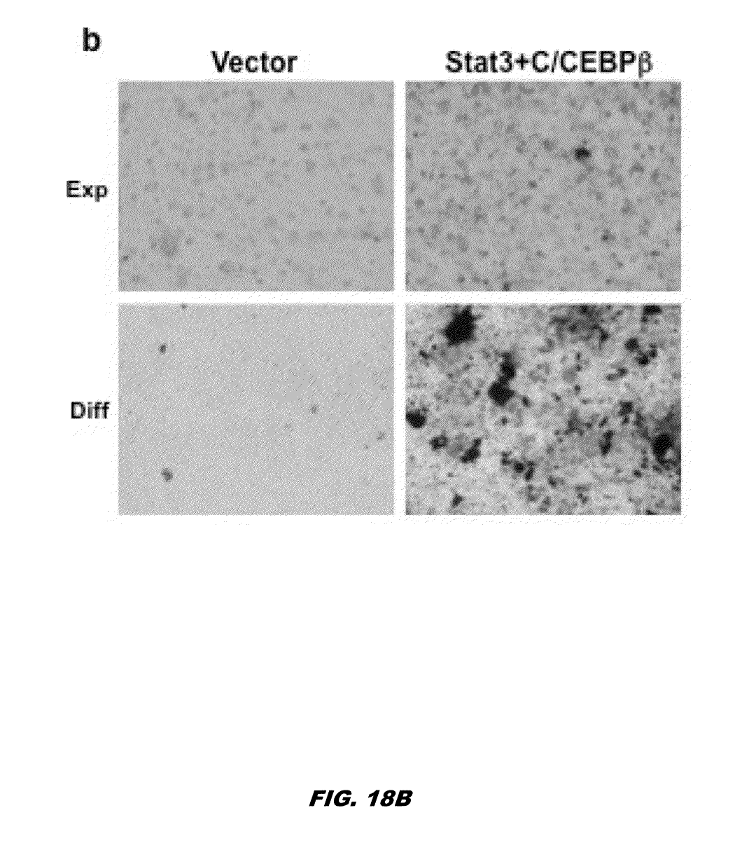

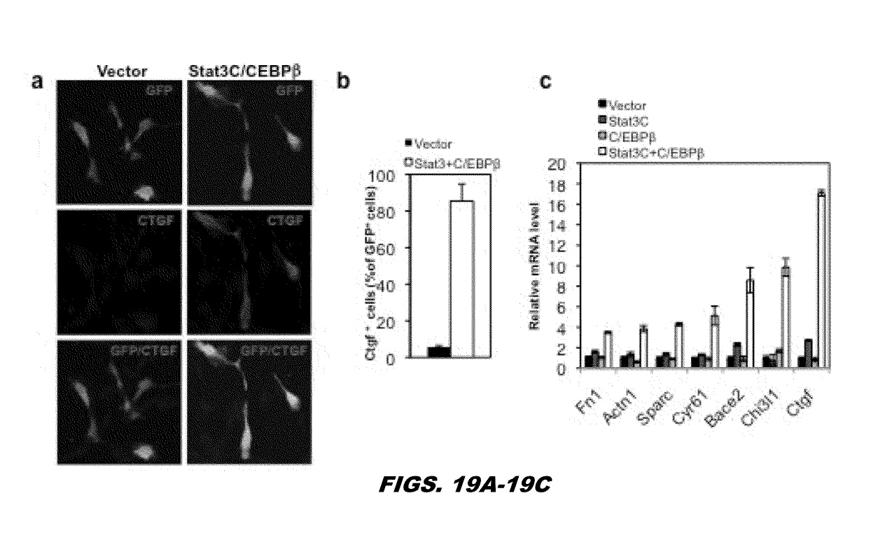

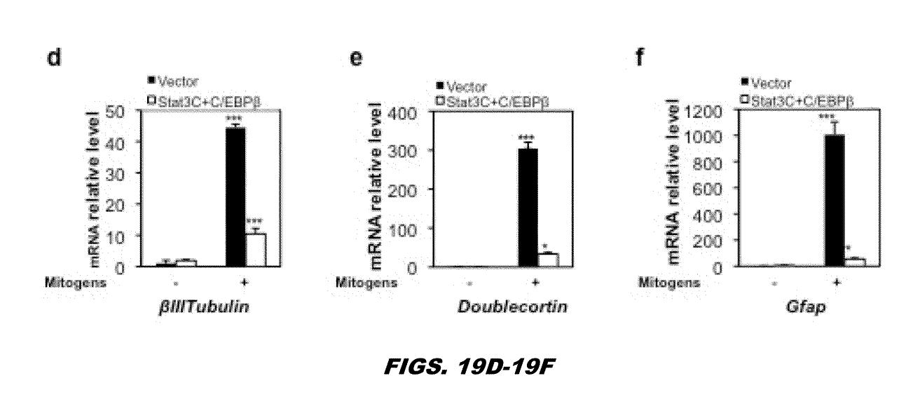

The morphological changes were associated with gain of the expression of the mesenchymal marker proteins SMA and fibronectin and induced mRNA expression of the mesenchymal genes Chi311/YKL40, Acta2/SMA, CTGF and OSMR. However, the individual expression of Stat3C or C/EBPβ was generally insufficient to induce either mesenchymal marker proteins or expression of mesenchymal genes. Rather than triggering differentiation along the neuronal lineage, removal of mitogens to Stat3C/C/EBPβ-expressing C17.2 cells resulted in further increase of the expression of mesenchymal genes and complete acquisition of mesenchymal features such as positive alcian blue staining, a specific assay for chondrocyte differentiation (FIG. 18A-B, FIG. 26A-B). Consistent with the cellular properties conferred by mesenchymal transformation to multiple cell types, we found that the expression of Stat3C and C/EBPβ robustly promoted migration in a wound assay and triggered invasion through the extracellular matrix in a Matrigel invasion assay (FIG. 5C-D). Invasion through Matrigel by C17.2 was stimulated by Stat3C and C/EBPβ in the absence of mitogens or in the presence of PDGF, a known inducer of cell migration, therefore indicating that the Stat3C/C/EBPβ-induced migration and invasion are likely cell intrinsic effects (FIG. 5D). Next, it was sought to establish the effects of C/EBPβ and Stat3 in primary NSCs. NSCs isolated from the mouse cortex at embryonic day 13 were cultured and infected with retroviruses expressing Stat3C together with a puromycin-resistance gene and/or C/EBPβ together with a green fluorescence protein (GFP). Also in this primary system the combined but not the individual expression of Stat3C and C/EBPβ efficiently induced mesenchymal marker proteins and mesenchymal gene expression (FIG. 19A-C). Conversely, Stat3C and C/EBPβ abolished differentiation along the neuronal and glial lineages that is normally triggered in NSCs by removal of mitogens (EGF and bFGF) from the medium (FIG. 19D-F). The C/EBPβ/Stat3C-induced mesenchymal transformation of primary NSCs was associated with withdrawal from cell cycle. Thus, the combined introduction of active C/EBPβ and Stat3 in NSCs prevents differentiation along the normal neural lineages and triggers reprogramming toward an aberrant mesenchymal lineage.

C/EBPβ and Stat3 are Essential for Mesenchymal Transformation and Aggressiveness of Human Glioma Cells In Vitro, in the Mouse Brain and in Primary Human Tumors.

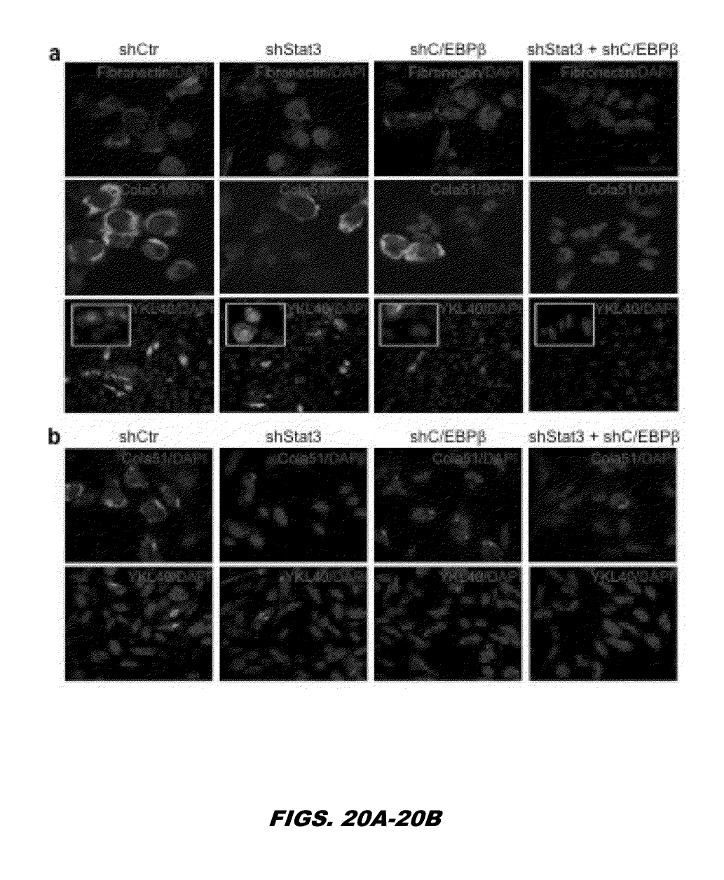

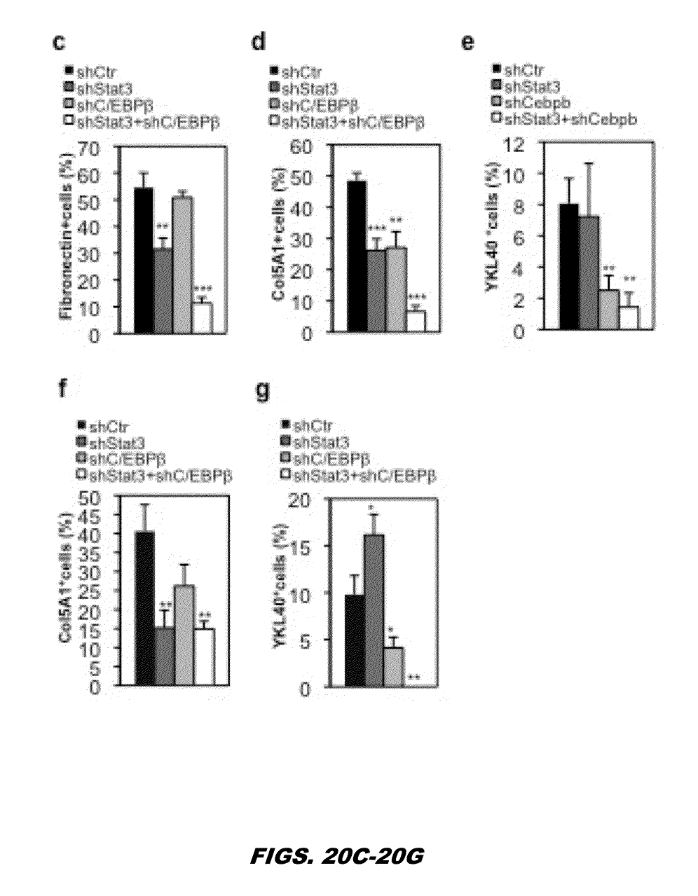

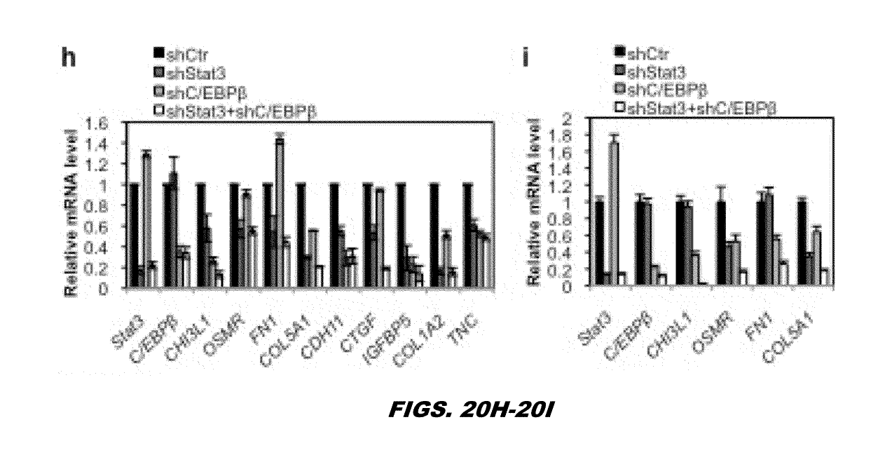

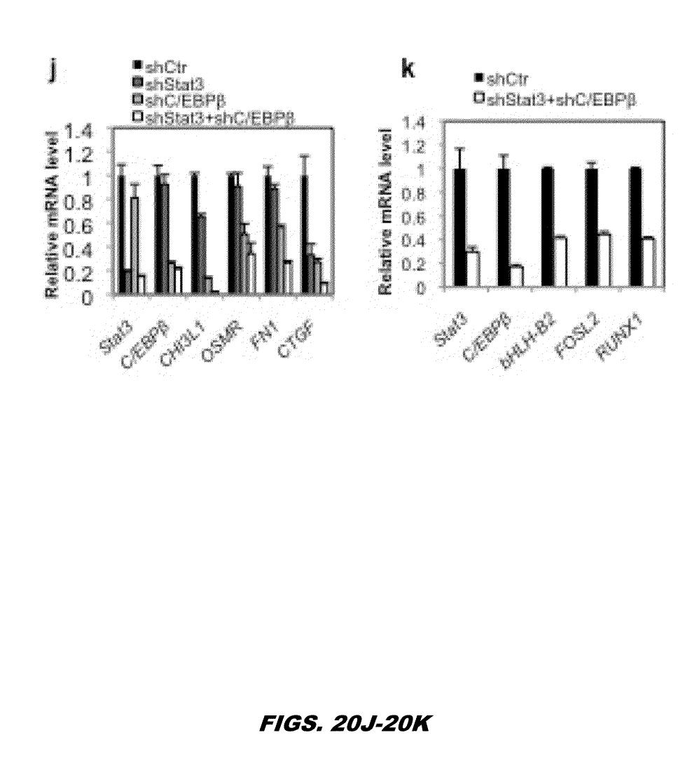

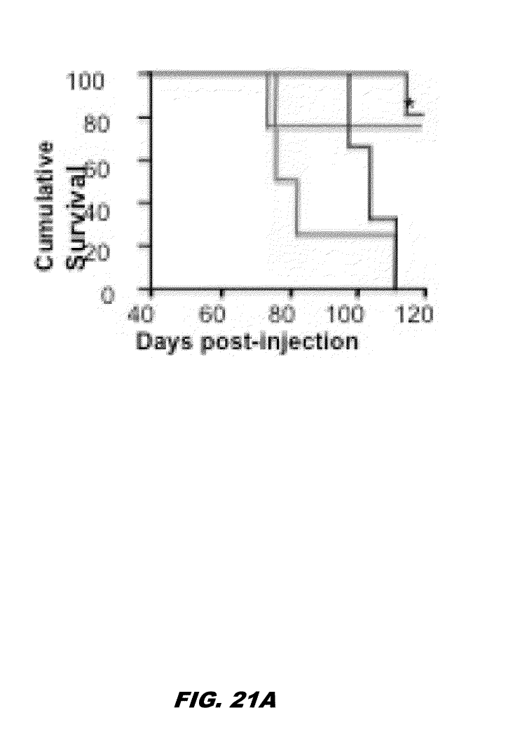

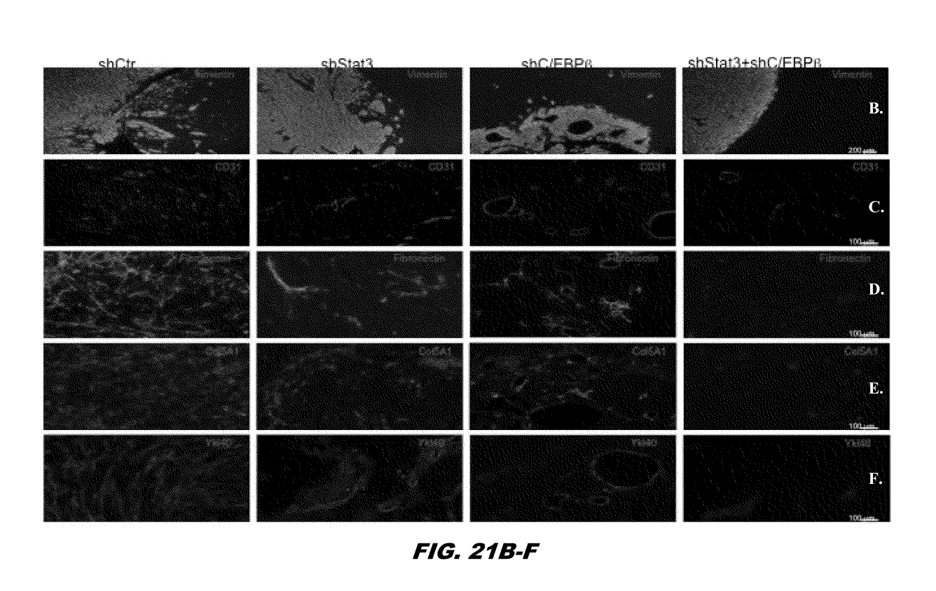

To assess the significance of constitutive C/EBPβ and Stat3 in the cells responsible for brain tumor growth in humans, it was sought to abolish the expression of C/EBPβ and Stat3 in cells freshly derived from primary human GBM and grown in serum-free medium, a condition optimal for retention of stem-like properties and tumor initiating ability (GBM-BTICs, see FIG. 7E AND FIG. 21) {Lee, 2006; herein incorporated by reference in its entirety}. Transduction of GBM-BTICs cultures derived from two GBM patients (BTSC-20 and BTSC-3408) with specific shRNA-carrying lentiviruses silenced endogenous C/EBPβ and Stat3 and efficiently eliminated expression of mesenchymal genes and depleted the tumor cells of the mesenchymal marker proteins fibronectin, collagen-5A1 and YKL40 (FIG. 20A-D, FIG. 20H, and FIG. 20I). Individual silencing of C/EBPβ or Stat3 produced variable inhibitory effects with the silencing of C/EBPβ typically carrying the most severe consequences (see for example the quantitative analysis of YKL40 staining in FIG. 20D). Combined or individual silencing of C/EBPβ and Stat3 in the human glioma cell line SNB19 produced effects similar to those observed in GBM-BTICs (FIG. 20E-G, FIG. 20J).

Next, it was considred whether loss of C/EBPβ and Stat3 in glioma cells reduced tumor aggressiveness in vitro and in vivo. First, it was found that silencing of the two TFs in SNB19 and GBM-BTICs eliminated >70% of their ability to invade through Matrigel (FIG. 22A, FIG. 7E). Then, the impact of C/EBPβ and Stat3 knockdown for brain tumorigenesis in vivo was determined. SNB19 cells transduced with non-targeting control shRNA lentivirus or shRNA targeting C/EBPβ and/or Stat3 were xenografted into the striatum of immunocompromised mice. Efficient tumor formation was observed in all mice injected with shRNA control and shStat3 cells. However, only one of four mice from the shC/EBPβ and one of five mice from the shC/EBPβ+shStat3 groups developed tumors after 120 days from the injection (FIG. 22B). The histologic analysis demonstrated high-grade tumors, which displayed peripheral invasion of the surrounding brain as single cells and cell clusters in the shRNA control group as shown by the staining pattern produced by a human specific vimentin antibody (FIG. 22C). Staining for the endothelial marker CD31 revealed marked vascularization in the shRNA control group of tumors. Conversely, the single tumor in the shC/EBPβ+shStat3 group grew well circumscribed and was less angiogenic. Tumors in the shStat3 group and the single tumor in the shC/EBPβ group had an intermediate growth pattern and limited angiogenesis (FIG. 22C-D). Consistent with the notion that the expression of mesenchymal markers correlates with brain tumor aggressiveness, it was found that staining for fibronectin, collagen-5A1 and YKL40 was readily detected in the tumors from the control group but absent or barely detectable in the single tumors from the shC/EBPβ and shC/EBPβ+shStat3 groups. Tumors derived from shStat3 cells displayed an intermediate phenotype with reduced expression of mesenchymal markers compared with tumors in the shcontrol group but higher than that observed in the tumors in the shC/EBPβ and shC/EBPβ+shStat3 groups (shcontrol>shStat3>shC/EBPβ>shC/EBPβ+shStat3).

Intracranial transplantation of GBM-BTICs transduced with shRNA control lentivirus produced extremely invasive tumor cell masses extending through the corpus callosum to the controlateral brain. Combined knockdown of C/EBPβ and Stat3 led to a significant decrease of the tumor area and tumor cell density as evaluated by human vimentin staining (FIG. 21B), markedly reduced the proliferation index (FIG. 21A) and abolished the expression of mesenchymal markers fibronectin and collagen-5A1 (FIG. 21D-E).

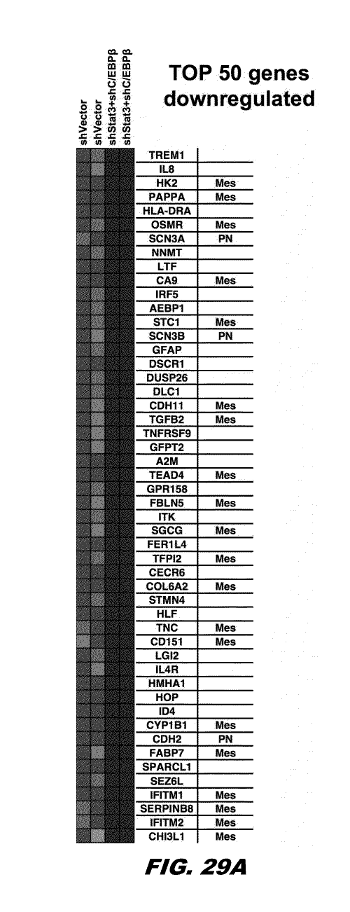

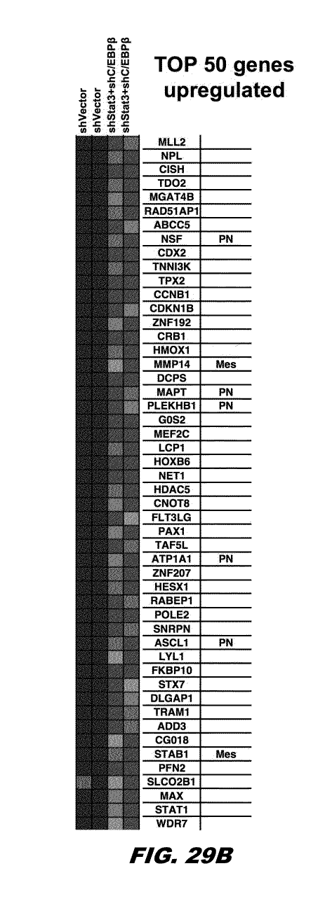

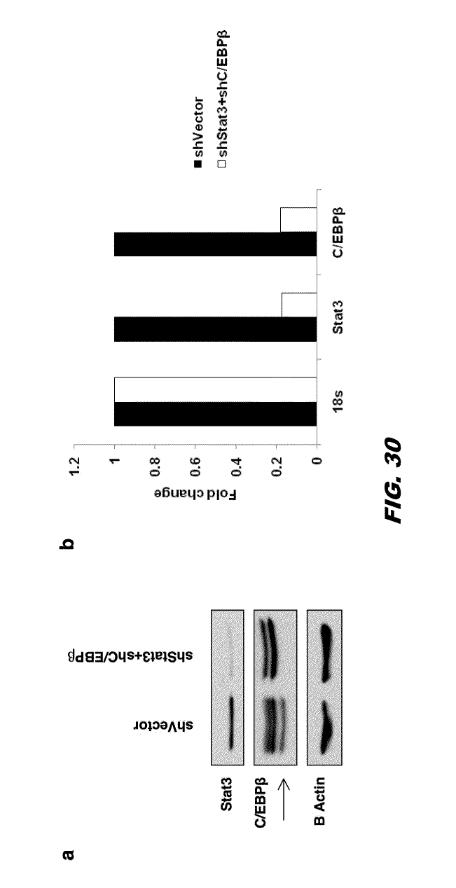

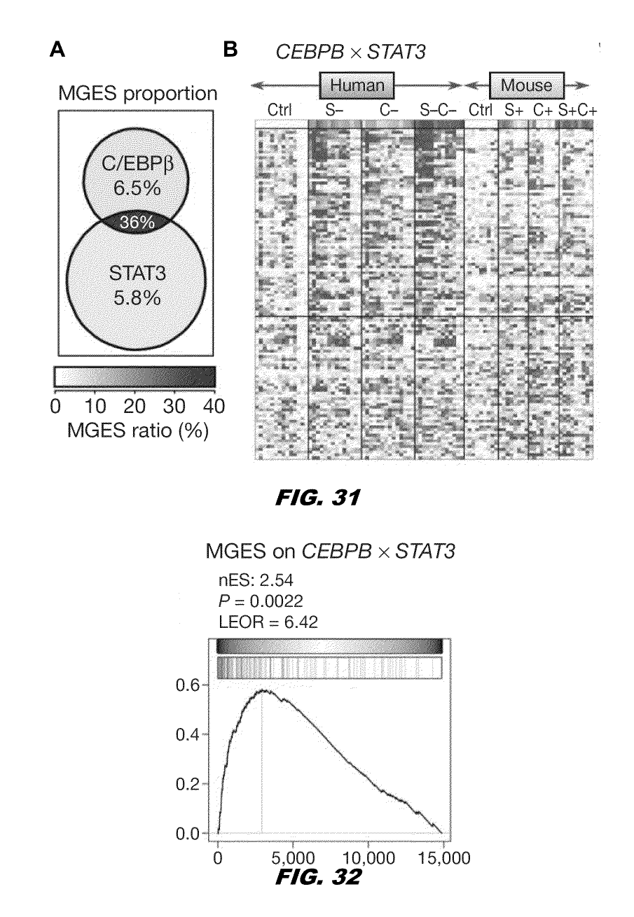

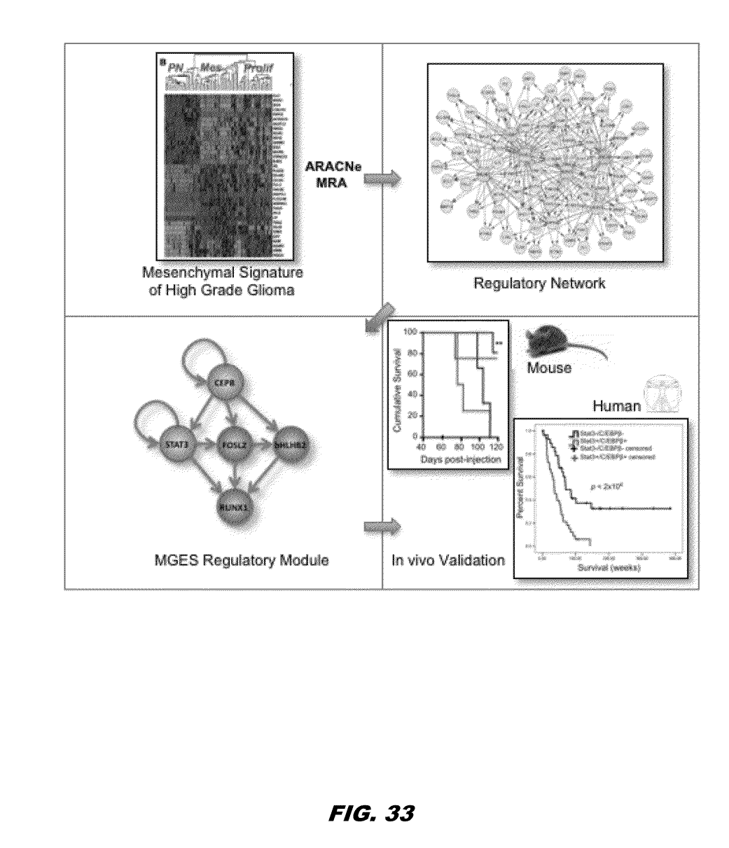

As final test for the significance of the expression of C/EBPβ and Stat3 for the mesenchymal phenotype and aggressiveness of human glioma, an immunohistochemical analysis was conducted for C/EBPβ and active, phospho-Stat3 in human tumor specimens, and the expression of these TFs was compared with YKL-40 (a well-established mesenchymal protein expressed in primary human GBM) {Nigro, 2005; Pelloski, 2005; each herein incorporated by reference in its entirety} and patient outcome in a collection of 62 newly diagnosed GBMs (FIG. 29A-B). FET analysis showed that expression of either C/EBPβ or Stat3 were significantly associated with YKL-40 expression (C/EBPβ, p=4.9×10⁻⁵; Stat3, p=2.2×10⁻⁴). However, the association was higher when double positive tumors (C/EBPβ+/Stat3+) were compared to double negatives (C/EBPβ−/Stat3−, p=2.7×10⁻⁶). Furthermore, double positive tumors were associated with markedly worse clinical outcome than tumors that were either single or double negatives (log-rank test, p=0.0002, FIG. 21E). Positivity for either of the two TFs remained predictive of negative outcome but with lower statistical strength than double positivity (C/EBPβ, p=0.0022; Stat3, p=0.0017). Together, the above results provide compelling indication that the activities of C/EBPβ and Stat3 are essential to maintain mesenchymal properties and aggressiveness of human glioma, and mark the worst survival group of GBM patients.

Discussion

Recent progress in systems biology has allowed the reconstruction of cellular networks proposed to play important functions in various phenotypic states, including cancer {Ergun, 2007; Rhodes, 2005; each herein incorporated by reference in its entirety}. However, network-based methods have yet to identify MRs of predefined tumor phenotypes that could withstand rigorous experimental validation. Similarly, synergistic/cooperative regulations of human phenotypes are virtually unexplored using network-based approaches. Here, it is shown that context-specific inference of a regulatory network in HGGs can be used to identify a transcriptional regulatory module that controls the expression of genes associated with the mesenchymal signature and poorest-prognosis of HGGs. Two of the module TFs, C/EBPβ and Stat3, were further characterized as first level controllers of module activity, via a large number of FF loops, and cooperative/synergistic initiators and MRs of the MGES. FF loops contribute to stabilizing positive regulation of the signature and to making its activity relatively insensitive to short regulatory fluctuations{Kalir, 2005; Milo, 2002, Science; each herein incorporated by reference in its entirety}.

In the proposed approach presented here, the traditional paradigm of gene expression profile based cancer research, yielding long lists of differentially expressed genes (i.e., cancer signatures), becomes a starting point for a cellular-network analysis where a causal regulatory model identifies the TFs that control the signatures and related phenotypes. As shown, the stability of the MRs across distinct datasets surpasses by far that of the signature genes. Indeed, poor overlap of cancer signatures and lack of validation across distinct datasets has been a long-standing concern {Ein-Dor, 2005; herein incorporated by reference in its entirety}. Yet the new approach produced virtually identical regulatory MR modules when applied to two completely distinct datasets and signatures associated with poor-prognosis in HGGs. Conversely, attempts to test several more conventional statistical association methods failed to identify the two MRs. This suggests that enrichment analysis of ARACNe-inferred TF regulons is specifically useful for the identification of MRs of tumor-related phenotypes. Due to the hyperexponential complexity in the number of parent regulators, other graph-theoretical methods such as Bayesian Networks may be less suited to explore regulatory modules where a large number of TFs cooperatively and synergistically determine signature regulation. The results do not exclude that such approaches may however provide further fine-grain regulatory insight once the number of candidate MRs is reduced to a handful by methods such as those proposed here. Yet, once a relatively small number of TFs is identified, direct experimental validation is feasible and will provide more conclusive results, as shown here.

While such an approach is of general applicability, it also presents some limitations. For instance, the activity of some TFs may be modulated only post-translationally, thus preventing the identification of their targets by ARACNe. Furthermore, due to false negatives, the regulons of some TFs may be too small to detect statistically significant enrichment, thus preventing their identification as potential MRs. The latter is partially mitigated by the fact that TFs with small regulons may be less likely to produce the broad regulatory changes associated with phenotypic transformations.

The experimental follow-up established that C/EBPβ and Stat3 are sufficient in NSCs and necessary in human glioma cells for mesenchymal transformation. Interestingly, C/EBPβ and Stat3 are expressed in the developing nervous system {Barnabe-Heider, 2005; Bonni, 1997; Nadeau, 2005; Sterneck, 1998; each herein incorporated by reference in its entirety}. However, while Stat3 induces astrocyte differentiation and inhibits neuronal differentiation of neural stem/progenitor cells, C/EBPβ promotes neurogenesis and opposes gliogenesis {He, 2005; Menard, 2002; Nakashima, 1999; Paquin, 2005; each herein incorporated by reference in its entirety}. How can the combined activity of C/EBPβ and Stat3 promote differentiation toward an aberrant lineage (mesenchymal) and oppose the genesis of the normal neural lineages (neuronal and glial)? Without being bound by theory, it is proposed that mesenchymal transformation results from concurrent activation of two conflicting transcriptional regulators normally operating to funnel opposing signals (neurogenesis vs. gliogenesis). This scenario is intolerable by normal neural stem/progenitor cells whereas it operates to permanently drive the mesenchymal phenotype in the context of the genetic and epigenetic changes that accompany high-grade gliomagenesis (EGFR amplification, PTEN loss, Akt activation, etc.) {Phillips, 2006; herein incorporated by reference in its entirety}.

The finding that C/EBPβ/Stat3C-expressing NSCs become unable to differentiate along the default neuronal lineage and lose expression of the normal proneural signature genes reflects the mutually exclusive expression of the proneural and mesenchymal signatures observed in primary GBM {Phillips, 2006; herein incorporated by reference in its entirety}. Without being bound by theory, it is proposed that the neuroepithelial to mesenchymal reprogramming induced by C/EBPβ and Stat3 recapitulates the epithelial to mesenchymal transition frequently described in epithelial neoplasms undergoing progression toward a more invasive and metastatic tumor type {Tarin, 2005; herein incorporated by reference in its entirety}. Thus, an exciting implication of this work is that, by acting upstream of the mesenchymal genes, C/EBP/Stat3-mediated transcription reprograms the cell fate of NSCs toward an aberrant “mesenchymal” lineage. In the context of other genetic and epigenetic alterations, this transformation triggers the most aggressive properties of malignant brain tumors, namely invasion and neo-angiogenesis. Since the expression of C/EBPβ and Stat3 in human glioma cells is essential to maintain the tumor initiating capacity and the ability to invade the normal brain, the two TFs provide important clues for diagnostic and pharmacological intervention. Consistent with this notion, the combined expression of C/EBPβ and Stat3 is linked to the mesenchymal state of primary GBM and provides an excellent prognostic biomarker for tumor aggressiveness.

In conclusion, the first evidence that computational systems biology methods can be effectively used to infer MRs that choreograph the malignant transformation of a human cell is presented. This is a general new paradigm that will be applicable to the dissection of normal and pathologic phenotypic states.

Methods

ARACNe Network Reconstruction.

ARACNe (Algorithm for the Reconstruction of Accurate Cellular Networks), an information-theoretic algorithm for inferring transcriptional interactions, was used to identify a repertoire of candidate transcriptional regulators of the MGES genes. Expression profiles used in the analysis were previously characterized using Affymetrix HU-133A microarrays and preprocessed by MAS 5.0 normalization procedure {Phillips, 2006; herein incorporated by reference in its entirety}. First, candidate interactions between a TF (x) and its potential target (y) are identified by computing pairwise mutual information, MI[x; y], using a Gaussian kernel estimator {Margolin, 2006; herein incorporated by reference in its entirety} and by thresholding the mutual information based on the null-hypothesis of statistical independence (p<0.05 Bonferroni corrected for the number of tested pairs). Then, indirect interactions are removed using the data processing inequality, a well known property of the mutual information. For each TF-target pair (x, y) a path through any other TF (z) was considered and any interaction such that MI[x; y]<min(MI[x; z], MI[y; z]) was removed.

Transcription Factor Classification.

To identify human transcription factors (TFs), the human genes annotated as “transcription factor activity” in Gene Ontology and the list of TFs from TRANSFAC were selected. From this list, general TFs (e.g. stable complexes like polymerases or TATA-box-binding proteins) were removed, and some TFs not annotated by GO were added, producing a final list of 928 TFs that were represented on the HG-U133A microarray gene set.

Master Regulator Analysis.

The MRA has two steps. First, for each TF its MGES-enrichment is computed as the p-value of the overlap between the TF-regulon and the MGES genes, assessed by Fisher Exact Test (FET). Since FET depends on regulon size, it can be used to assess MGES-enriched TFs but not to rank them. MGES-enriched TFs are thus ranked based on the total number of MGES genes in their regulon, under the assumption that TFs controlling a larger fraction of MGES genes will be more likely to determine signature activity.

Stepwise Linear Regression (SLR) Analysis.

A regulatory program for each MGES gene was computed as follows: the log₂ expression of the i-th MGES gene was considered as the response variable and the log₂-expression of the TFs as the explanatory variables in the linear model log xᵢ=Σαᵢⱼ log fⱼ+βᵢⱼ {Tegner, 2003}. Here, fⱼ represents the expression of the j-th TF in the model and the (αᵢⱼ, βᵢⱼ) are linear coupling coefficients computed by standard regression analysis. TFs are iteratively added to the model, by choosing each time the one producing the smallest relative error E=Σ|xᵢ−xᵢ₀|/xᵢ₀ between predicted and observed target expression. This is repeated until the decrease in relative error is no longer statistically significant, based on permutation testing. To avoid excessive multiple hypothesis testing correction, TFs were chosen only among the following: (a) the 55 inferred by ARACNe at FDR <0.05 and (b) TFs whose DNA binding signature was significantly enriched in the proximal promoter of the MGES genes and that are expressed in the dataset, based on the coefficient of variation (CV≧0.5). TFs were then ranked based on the number of MGES target they regulated, with the average Linear-Regression coefficient providing additional insight.

Cell Lines and Cell Culture Conditions.

SNB75, SNB19, 293T and Phoenix cell lines were grown in DMEM plus 10% Fetal Bovine Serum (FBS, Gibco/BRL). GBM-derived BTICs were grown as neurospheres in Neurobasal media (Invitrogen) containing N2 and B27 supplements (Invitrogen), and human recombinant FGF-2 and EGF (50 ng/ml each; Peprotech). Murine neural stem cells (mNSCs) (from an early passage of clone C17.2) (27-29; each herein incorporated by reference in its entirety) were cultured in DMEM plus 10% heat inactivated FBS, (Gibco/BRL), 5% Horse serum (Gibco/BRL) and 1% L-Glutamine (Gibco/BRL). Neuronal differentiation of mNSCs was induced by growing cells in DMEM supplemented with 0.5% Horse serum. For chondrocyte differentiation, cells were treated with STEMPRO chondrogenesis differentiation kit (Gibco/BRL) for 20 days.

Primary murine neural stem cells were isolated from E13.5 mouse telencephalon and cultured in the presence of FGF-2 and EGF (20 ng/ml each) as described {Bachoo, 2002; herein incorporated by reference in its entirety} Differentiation of neural stem cells was induced by culturing neurospheres on laminin-coated dishes in NSC medium in the absence of growth factors. mNSC expressing Stat3C and C/EBPβ, were generated by retroviral infections using supernatant from Phoenix ecotropic packaging cells transfected with pBabe-Stat3C-FLAG and/or pLZRS-T7-His-C/EBPβ-2-IRES-GFP.

Promoter Analysis and Chromatin Immunoprecipitation (ChIP).

Promoter analysis was performed using the MatInspector software (www.genomatix.de; herein incorporated by reference in its entirety). A sequence of 2 kb upstream and 2 kb downstream from the transcription start site was analyzed for the presence of putative binding sites for each TFs. Primers used to amplify sequences surroundings the predicted binding sites were designed using the Primer3 software (http://frodo.wi.mit.edu/cgi-bin/primer3/primer3_www.cgi; herein incorporated by reference in its entirety) and are listed in Table 15.

Chromatin immunoprecipitaion was performed as described in Frank, 2001; herein incorporated by reference in its entirety. SNB75 cells lysates were precleared with Protein A/G beads (Santa Cruz) and incubated at 4° C. overnight with 1 μl of polyclonal antibody specific for C/EBPβ (sc-150, Santa Cruz), Stat3 (sc-482, Santa Cruz), FosL2 (Fra2, sc-604, Santa Cruz), bHLH-B2 (A300-649A, BETHYL Laboratories), or normal rabbit immunoglobulins (Santa Cruz). DNA was eluted in 200 μl of water and 1 μl was analyzed by PCR with Platinum Taq (Invitrogen). For primary GBM samples, 30 mg of frozen tissue was transferred in a tube with 1 ml of culture medium, fixed with 1% formaldehyde for 15 min and stopped with 0.125 M glycine for 5 min. Samples were centrifuged at 4000 rpm for 2 min, washed twice and diluted in PBS. Tissues were homogenized using a pestle and suspended in 3 ml of ice-cold immunoprecipitation buffer with protease inhibitors and sonicated. ChIP was then performed as described above.

QRT—PCR and Microarray Analysis.

RNA was prepared with RiboPure kit (Ambion), and used for first strand cDNA synthesis using random primers and SuperScriptll Reverse Transcriptase (Invitrogen). QRT-PCR was performed using Power SYBR Green PCR Master Mix (Applied Biosystems). Primers are listed in Table 16. QRT-PCR results were analyzed by the ΔΔCT method (Livak & Schmittgen, Methods 25:402, 2001; herein incorporated by reference in its entirety) using GAPDH or 18S as housekeeping genes.

RNA amplification for Array analysis was performed with Illumina TotalPrep RNA Amplification Kit (Ambion). 1.5 μg of amplified RNA was hybridized on Illumina HumanHT-12v3 or MouseWG-6 expression BeadChip according to the manufacturer's instructions. Hybridization data was obtained with an iScan BeadArray scanner (Illumina) and pre-processed by variance stabilization and robust spline normalization implemented in the lumi package under the R-system (Du, P., Kibbe, W. A. and Lin, S. M., (2008) ‘lumi: a pipeline for processing Illumina microarray’, Bioinformatics 24(13):1547-1548; herein incorporated by reference in its entirety).

Immunofluorescence and Immunohistochemistry.

Immunofluorescence staining was performed as previously described {Rothschild, 2006; herein incorporated by reference in its entirety}. Primary antibodies and dilutions were: SMA (mouse monoclonal, Sigma, 1:200), Fibronectin (mouse monoclonal, BD Biosciences, 1:200), Tau (rabbit polyclonal, Dako, 1:400), βIIITubulin (mouse monoclonal, Promega, 1:1000), CTGF (rabbit polyclonal, Santa Cruz, 1:200), YKL40 (rabbit polyclonal, Quidel, 1:200) and Col5A1 (rabbit polyclonal, Santa Cruz, 1:200). Confocal images acquired with a Zeiss Axioscop2 FS MOT microscope were used to score positive cells. At least 500 cells were scored for each sample. Quantification of the fibronectin intensity staining in mNSC was performed using NIH Image J software (http://rsb.info.nih.gov/ij/, NIH, USA; herein incorporated by reference in its entirety). The histogram of the intensity of fluorescence of each point of a representative field for each condition was generated. The fluorescence intensity of three fields from three independent experiments was scored, standardized to the number of cells in the field and divided by the intensity of the vector. For immunostaining of xenograft tumors, mice were perfused trans-cardially with 4% PFA, brains were dissected and post-fixed for 48 h in 4% PFA. Immunostaining was performed as previously described {Zhao, 2008; herein incorporated by reference in its entirety}. Primary antibodies and dilutions were fibronectin (mouse moclonal, BD Bioscences, 1; 100), Col5A1 (rabbit polyclonal, Santa Cruz, 1:100), YKL40 (rabbit polyclonal, Quidel, 1; 100), human vimentin (mouse monoclonal, Sigma, 1:50), Ki67 (rabbit polyclonal, Novocastra laboratories, 1:1000). Quantification of the tumor area was obtained by measuring the human vimentin positive area in the section using the NIH Image J software (http://rsb.info.nih.gov/ij/, NIH, USA; herein incorporated by reference in its entirety). Five tumors for each group were analyzed. For quantification of Ki67, the percentage of positive cells was scored in 5 tumors per each group. In histogram values represents the mean values; error bars are standard deviations. Statistical significance was determined by t test (with Welch's Correction) using GraphPad Prism 4.0 software (GraphPad Inc., San Diego, Calif.). Immunohistochemistry of primary human GBM was performed as previously described {Simmons, 2001; herein incorporated by reference in its entirety}. The primary antibodies and dilutions were anti-YKL-40 (rabbit polyclonal, Quidel, 1:750), anti C/EBPβ, (rabbit polyclonal, Santa Cruz, 1:250) and anti-p-Stat3 (rabbit monoclonal, Cell Signaling, 1; 25), Scoring for YKL-40 was based on a 3-tiered system, where 0 was <5% of tumor cells positive, 1 was 5-30% positivity and 2 was >30% of tumor cells positive. Scores of 1 and 2 were later collapsed into a single value for display purposes on Kaplan-Meier curves. Associations between C/EBPβ/Stat3 and YKL-40 were assessed using the Fisher exact test (FET). Associations between C/EBPβ/Stat3 and patients survival were assessed using the log-rank (Mantel-Cox) test of equality of survival distributions.

Migration and Invasion Assays.

For the wound assay testing migration, mNSCs were plated in 60 mm dishes and grown until 95% confluence. A scratch of approximately 1000 μm was made with a P1000 pipet tip and images were taken every 24 h with an inverted microscope. For the Matrigel invasion assay, mNSCs and SNB19 (1×10⁴) were added to the upper compartment of a 24 well BioCoat Matrigel Invasion Chamber (BD Bioscences) in serum free DMEM. The lower compartment of the chamber was filled with DMEM containing either 0.5% horse serum or 20 μg/ml PDGF-BB (R&D systems) as chemoattractant. After 24 h, invading cells were fixed, stained according to the manufacturer's instructions and counted. For GBM-derived BTICs, 5×10⁴ cells were plated on the upper chamber in the absence of growth factors. In the lower compartment Neurobasal medium containing B27 and N2 supplements plus 20 μg/ml PDGF-BB (R&D systems) was used as chemoattractant.

Lentivirus Infection.

Lentiviral expression vectors carrying shRNAs were purchased from Sigma. The sequences are listed in Table 17. To generate lentiviral particles, each shRNA expression plasmid was co-transfected with pCMV-dR8.91 and pCMV-MD2.G vectors into human embryonic kidney 293T cells using Fugene 6 (Roche). Lentiviral infections were performed as described {Zhao, 2008; herein incorporated by reference in its entirety}.

Intracranial Injection.

Intracranial injection of SNB19 glioma cell line and GBM-derived BTICs was performed in 6-8 weeks NOD/SCID mice (Charles River laboratories) in accordance with guidelines of the International Agency for Reserch on Cancer's Animal Care and Use Committee. Briefly, 48 h after lentiviral infection, 2×10⁵ SNB19 or 3×10⁵ BTICs were injected 2 mm lateral and 0.5 mm anterior to the bregma, 3 mm below the skull. Mice were monitored daily and sacrificed when neurological symptoms appeared. Kaplan-Meier survival curve of the mice injected with SNB19 glioma cells was generated using the DNA Statview software package (AbacusConcepts, Berkeley Calif.).

TABLE 3A
Table 3A. Genes in the MGES signature.

| AffyID | Gene Symbol | Gene ID | MRA | Illumina |
|---|---|---|---|---|
| 200660_at | S100A11 | 6282 | * | |
| 200808_s_at | ZYX | 7791 | * | * |
| 200859_x_at | FLNA | 2316 | * | * |
| 200879_s_at | EPAS1 | 2034 | * | * |
| 200974_at | ACTA2 | 59 | * | |
| 201058_s_at | MYL9 | 10398 | * | |
| 201169_s_at | BHLHB2 | 8553 | * | * |
| 201204_s_at | RRBP1 | 6238 | * | * |
| 201315_x_at | IFITM2 | 10581 | * | * |
| 201389_at | ITGA5 | 3678 | * | * |
| 201473_at | JUNB | 3726 | * | * |
| 201474_s_at | ITGA3 | 3675 | * | * |
| 201645_at | TNC | 3371 | * | * |
| 201666_at | TIMP1 | 7076 | * | * |
| 201750_s_at | ECE1 | 1889 | * | * |
| 202180_s_at | MVP | 9961 | * | * |
| 202627_s_at | SERPINE1 | 5054 | * | * |
| 202628_s_at | SERPINE1 | 5054 | * | * |
| 202637_s_at | ICAM1 | 3383 | * | * |
| 202638_s_at | ICAM1 | 3383 | * | * |
| 202669_s_at | EFNB2 | 1948 | * | * |
| 202765_s_at | FBN1 | 2200 | * | |
| 202771_at | FAM38A | 9780 | * | |
| 202827_s_at | MMP14 | 4323 | * | * |
| 202833_s_at | SERPINA1 | 5265 | * | * |
| 202856_s_at | SLC16A3 | 9123 | * | * |
| 202888_s_at | ANPEP | 290 | * | * |
| 202910_s_at | CD97 | 976 | * | * |
| 203370_s_at | PDLIM7 | 9260 | * | |
| 203691_at | PI3 | 5266 | * | |
| 203729_at | EMP3 | 2014 | * | * |
| 203828_s_at | IL32 | 9235 | * | |
| 203835_at | LRRC32 | 2615 | * | |
| 203887_s_at | THBD | 7056 | * | * |
| 203888_at | THBD | 7056 | * | * |
| 204036_at | LPAR1 | 1902 | * | * |
| 204037_at | LPAR1 | 1902 | * | * |
| 204166_at | SBNO2 | 22904 | * | * |
| 204293_at | SGSH | 6448 | * | * |
| 204306_s_at | CD151 | 977 | * | * |
| 204879_at | PDPN | 10630 | * | |
| 204908_s_at | BCL3 | 602 | * | * |
| 204981_at | SLC22A18 | 5002 | * | * |
| 205226_at | PDGFRL | 5157 | * | * |
| 205266_at | LIF | 3976 | * | * |
| 205418_at | FES | 2242 | * | * |
| 205463_s_at | PDGFA | 5154 | * | |
| 205547_s_at | TAGLN | 6876 | * | * |
| 205572_at | ANGPT2 | 285 | * | * |
| 205580_s_at | HRH1 | 3269 | * | * |
| 205729_at | OSMR | 9180 | * | * |
| 205936_s_at | HK3 | 3101 | * | * |
| 206178_at | PLA2G5 | 5322 | * | * |
| 206306_at | RYR3 | 6263 | * | * |
| 206359_at | SOCS3 | 9021 | * | * |
| 207714_s_at | SERPINH1 | 871 | * | * |
| 208394_x_at | ESM1 | 11082 | * | * |
| 208637_x_at | ACTN1 | 87 | * | * |
| 208789_at | PTRF | 284119 | * | * |
| 208790_s_at | PTRF | 284119 | * | * |
| 209356_x_at | EFEMP2 | 30008 | * | * |
| 209359_x_at | RUNX1 | 861 | * | |
| 209360_s_at | RUNX1 | 861 | * | |
| 209395_at | CHI3L1 | 1116 | * | * |
| 209396_s_at | CHI3L1 | 1116 | * | * |
| 209626_s_at | OSBPL3 | 26031 | * | * |
| 209663_s_at | ITGA7 | 3679 | * | * |
| 210287_s_at | FLT1 | 2321 | * | * |
| 210510_s_at | NRP1 | 8829 | * | * |
| 210735_s_at | CA12 | 771 | * | * |
| 210762_s_at | DLC1 | 10395 | * | * |
| 210772_at | FPR2 | 2358 | * | * |
| 210845_s_at | PLAUR | 5329 | * | * |
| 210992_x_at | FCGR2C | 9103 | * | |
| 211012_s_at | PML | 5371 | * | |
| 211148_s_at | ANGPT2 | 285 | * | * |
| 211160_x_at | ACTN1 | 87 | * | * |
| 211429_s_at | SERPINA1 | 5265 | * | * |
| 211564_s_at | PDLIM4 | 8572 | * | * |
| 211668_s_at | PLAU | 5328 | * | * |
| 211844_s_at | NRP2 | 8828 | * | * |
| 211924_s_at | PLAUR | 5329 | * | * |
| 211926_s_at | MYH9 | 4627 | * | |
| 211964_at | COL4A2 | 1284 | * | * |
| 211966_at | COL4A2 | 1284 | * | * |
| 211980_at | COL4A1 | 1282 | * | * |
| 211981_at | COL4A1 | 1282 | * | * |
| 212067_s_at | C1R | 715 | * | * |
| 212203_x_at | IFITM3 | 10410 | * | * |
| 212647_at | RRAS | 6237 | * | * |
| 212951_at | GPR116 | 221395 | * | * |
| 213746_s_at | FLNA | 2316 | * | * |
| 213895_at | EMP1 | 2012 | * | * |
| 214196_s_at | TPP1 | 1200 | * | * |
| 214660_at | PELO | 53918 | * | * |
| 214752_x_at | FLNA | 2316 | * | * |
| 214853_s_at | SHC1 | 6464 | * | * |
| 215498_s_at | MAP2K3 | 5606 | * | * |
| 215760_s_at | SBNO2 | 22904 | * | * |
| 215870_s_at | PLA2G5 | 5322 | * | * |
| 216331_at | ITGA7 | 3679 | * | * |
| 217867_x_at | BACE2 | 25825 | * | * |
| 217875_s_at | PMEPA1 | 56937 | * | * |
| 218272_at | TTC38 | 55020 | * | |
| 218424_s_at | STEAP3 | 55240 | * | * |
| 218880_at | FOSL2 | 2355 | * | * |
| 218983_at | C1RL | 51279 | * | * |
| 219025_at | CD248 | 57124 | * | * |
| 219042_at | LZTS1 | 11178 | * | * |
| 219566_at | PLEKHF1 | 79156 | * | * |
| 219869_s_at | SLC39A8 | 64116 | * | * |
| 220442_at | GALNT4 | 8693 | * | * |
| 220681_at | C22orf26 | 55267 | * | |
| 220975_s_at | C1QTNF1 | 114897 | * | * |
| 221293_s_at | DEF6 | 50619 | * | * |
| 221807_s_at | TRABD | 80305 | * | * |
| 221870_at | EHD2 | 30846 | * | * |
| 221898_at | PDPN | 10630 | * | |
| 221920_s_at | SLC25A37 | 51312 | * | * |
| 222206_s_at | NCLN | 56926 | * | |
| 222222_s_at | HOMER3 | 9454 | * | |
| 222528_s_at | SLC25A37 | 51312 | * | * |
| 222723_at | LOC727901 | 727901 | | |
| 222817_at | HSD3B7 | 80270 | | * |
| 223321_s_at | FGFRL1 | 53834 | | * |
| 223333_s_at | ANGPTL4 | 51129 | * | * |
| 223994_s_at | SLC12A9 | 56996 | | * |
| 224197_s_at | C1QTNF1 | 114897 | * | * |
| 224710_at | RAB34 | 83871 | | * |
| 224822_at | DLC1 | 10395 | * | * |
| 224942_at | PAPPA | 5069 | * | * |
| 225262_at | FOSL2 | 2355 | * | * |
| 225548_at | SHROOM3 | 57619 | | * |
| 225868_at | TRIM47 | 91107 | | * |
| 225869_s_at | UNC93B1 | 81622 | * | * |
| 225955_at | METRNL | 284207 | | * |
| 226328_at | KLF16 | 83855 | | * |
| 226401_at | PARP10 | 84875 | | |
| 226498_at | FLT1 | 2321 | * | * |
| 226621_at | FGG | 2266 | * | * |
| 226658_at | PDPN | 10630 | * | |
| 226722_at | FAM20C | 56975 | | * |
| 227055_at | METTL7B | 196410 | | * |
| 227272_at | C15orf52 | 388115 | | |
| 227325_at | LOC255783 | 255783 | | |
| 227345_at | TNFRSF10D | 8793 | * | |
| 227458_at | PDCD1LG1 | 29126 | | * |
| 227592_at | ALDH16A1 | 126133 | | * |
| 227697_at | SOCS3 | 9021 | * | * |
| 228498_at | B4GALT1 | 2683 | * | * |
| 229438_at | LOC100132244 | 100132244 | | |
| 229661_at | SALL4 | 57167 | | * |
| 230046_at | SPRED3 | 399473 | | * |
| 230283_at | NEURL2 | 140825 | | * |
| 230501_at | — | — | | |
| 231420_at | GGN | 199720 | | * |
| 231698_at | FLJ36848 | 647115 | | |
| 231876_at | TRIM56 | 81844 | | * |
| 232078_at | PVRL2 | 5819 | * | * |
| 232079_s_at | PVRL2 | 5819 | * | * |
| 232545_at | LRRC29 | 26231 | | |
| 232748_at | PAPPA | 5069 | * | * |
| 233695_s_at | CECR2 | 27443 | | * |
| 235417_at | SPOCD1 | 90853 | | |
| 235489_at | RHOJ | 57381 | | * |
| 238938_at | THADA | 63892 | * | * |
| 239507_at | LOC151300 | 151300 | | |
| 241645_at | MYO1D | 4642 | * | * |
| 243033_at | TWF1 | 5756 | | |
| 41469_at | PI3 | 5266 | * | |

TABLE 3B
Table 3B. Genes in the PNGES signature.

| | Gene | |
|---|---|---|
| AffyID | Symbol | Gene ID |
| 200612_s_at | AP2B1 | 163 |
| 200831_s_at | SCD | 6319 |
| 200946_x_at | GLUD1 | 2746 |
| 200965_s_at | ABLIM1 | 3983 |
| 201718_s_at | EPB41L2 | 2037 |
| 201830_s_at | NET1 | 10276 |
| 202022_at | ALDOC | 230 |
| 202178_at | PRKCZ | 5590 |
| 202455_at | HDAC5 | 10014 |
| 203146_s_at | GABBR1 | 2550 |
| 203381_s_at | APOE | 348 |
| 203382_s_at | APOE | 348 |
| 203485_at | RTN1 | 6252 |
| 203609_s_at | ALDH5A1 | 7915 |
| 203619_s_at | FAIM2 | 23017 |
| 203631_s_at | GPRC5B | 51704 |
| 203853_s_at | GAB2 | 9846 |
| 203928_x_at | MAPT | 4137 |
| 203929_s_at | MAPT | 4137 |
| 204072_s_at | FRY | 10129 |
| 204100_at | THRA | 7067 |
| 204134_at | PDE2A | 5138 |
| 204411_at | KIF21B | 23046 |
| 204513_s_at | ELMO1 | 9844 |
| 204749_at | NAP1L3 | 4675 |
| 204754_at | HLF | 3131 |
| 204762_s_at | GNAO1 | 2775 |
| 204953_at | SNAP91 | 9892 |
| 205050_s_at | MAPK8IP2 | 23542 |
| 205110_s_at | FGF13 | 2258 |
| 205278_at | GAD1 | 2571 |
| 205289_at | BMP2 | 650 |
| 205290_s_at | BMP2 | 650 |
| 205318_at | KIF5A | 3798 |
| 205330_at | MN1 | 4330 |
| 205358_at | GRIA2 | 2891 |
| 205575_at | C1QL1 | 10882 |
| 205730_s_at | ABLIM3 | 22885 |
| 205751_at | SH3GL2 | 6456 |
| 205754_at | F2 | 2147 |
| 205903_s_at | KCNN3 | 3782 |
| 205960_at | PDK4 | 5166 |
| 206103_at | RAC3 | 5881 |
| 206117_at | TPM1 | 7168 |
| 206137_at | RIMS2 | 9699 |
| 206196_s_at | RUNDC3A | 10900 |
| 206243_at | TIMP4 | 7079 |
| 206298_at | ARHGAP22 | 58504 |
| 206320_s_at | SMAD9 | 4093 |
| 206355_at | GNAL | 2774 |
| 206356_s_at | GNAL | 2774 |
| 206401_s_at | MAPT | 4137 |
| 206453_s_at | NDRG2 | 57447 |
| 206518_s_at | RGS9 | 8787 |
| 206604_at | OVOL1 | 5017 |
| 206785_s_at | KLRC1 | 3821 |
| 206850_at | RASL10A | 10633 |
| 207091_at | P2RX7 | 5027 |
| 207093_s_at | OMG | 4974 |
| 207210_at | GABRA3 | 2556 |
| 207276_at | CDR1 | 1038 |
| 207302_at | SGCG | 6445 |
| 207414_s_at | PCSK6 | 5046 |
| 207501_s_at | FGF12 | 2257 |
| 207723_s_at | KLRC3 | 3823 |
| 208017_s_at | MCF2 | 4168 |
| 208102_s_at | PSD | 5662 |
| 208552_at | GRIK4 | 2900 |
| 209283_at | CRYAB | 1410 |
| 209293_x_at | ID4 | 3400 |
| 209347_s_at | MAF | 4094 |
| 209504_s_at | PLEKHB1 | 58473 |
| 209558_s_at | HIP1R | 9026 |
| 209610_s_at | SLC1A4 | 6509 |
| 209611_s_at | SLC1A4 | 6509 |
| 209839_at | DNM3 | 26052 |
| 209889_at | SEC31B | 25956 |
| 209981_at | CSDC2 | 27254 |
| 209987_s_at | ASCL1 | 429 |
| 209988_s_at | ASCL1 | 429 |
| 209991_x_at | GABBR2 | 9568 |
| 210035_s_at | RPL5 | 6125 |
| 210222_s_at | RTN1 | 6252 |
| 210414_at | FLRT1 | 23769 |
| 210432_s_at | SCN3A | 6328 |
| 210657_s_at | SEPT4 | 5414 |
| 210753_s_at | EPHB1 | 2047 |
| 210815_s_at | CALCRL | 10203 |
| 211006_s_at | KCNB1 | 3745 |
| 211162_x_at | SCD | 6319 |
| 211184_s_at | USH1C | 10083 |
| 211203_s_at | CNTN1 | 1272 |
| 211484_s_at | DSCAM | 1826 |
| 211520_s_at | GRIA1 | 2890 |
| 211663_x_at | PTGDS | 5730 |
| 211679_x_at | GABBR2 | 9568 |
| 211708_s_at | SCD | 6319 |
| 211748_x_at | PTGDS | 5730 |
| 211819_s_at | SORBS1 | 10580 |
| 211898_s_at | EPHB1 | 2047 |
| 211925_s_at | PLCB1 | 23236 |
| 212187_x_at | PTGDS | 5730 |
| 212419_at | ZCCHC24 | 219654 |
| 212611_at | DTX4 | 23220 |
| 212812_at | SERINC5 | 256987 |
| 212884_x_at | APOE | 348 |
| 212914_at | CBX7 | 23492 |
| 213091_at | CRTC1 | 23373 |
| 213217_at | ADCY2 | 108 |
| 213222_at | PLCB1 | 23236 |
| 213411_at | ADAM22 | 53616 |
| 213433_at | ARL3 | 403 |
| 213486_at | COPG2IT1 | 53844 |
| 213549_at | SLC18A2 | 6571 |
| 213601_at | SLIT1 | 6585 |
| 213664_at | SLC1A1 | 6505 |
| 213724_s_at | PDK2 | 5164 |
| 213744_at | ATRNL1 | 26033 |
| 213824_at | OLIG2 | 10215 |
| 213825_at | OLIG2 | 10215 |
| 213841_at | — | — |
| 213880_at | LGR5 | 8549 |
| 213904_at | — | — |
| 213924_at | MPPE1 | 65258 |
| 214046_at | FUT9 | 10690 |
| 214071_at | MPPE1 | 65258 |
| 214111_at | OPCML | 4978 |
| 214162_at | LOC284244 | 284244 |
| 214251_s_at | NUMA1 | 4926 |
| 214279_s_at | NDRG2 | 57447 |
| 214376_at | — | — |
| 214434_at | HSPA12A | 259217 |
| 214487_s_at | RAP2A | 5911 |
| 214589_at | FGF12 | 2257 |
| 214680_at | NTRK2 | 4915 |
| 214762_at | ATP6V1G2 | 534 |
| 214834_at | PAR5 | 8123 |
| 214874_at | PKP4 | 8502 |
| 214914_at | FAM13C1 | 220965 |
| 214930_at | SLITRK5 | 26050 |
| 214952_at | NCAM1 | 4684 |
| 214954_at | SUSD5 | 26032 |
| 215306_at | — | — |
| 215323_at | LUZP2 | 338645 |
| 215444_s_at | TRIM31 | 11074 |
| 215469_at | — | — |
| 215522_at | SORCS3 | 22986 |
| 215687_x_at | PLCB1 | 23236 |
| 215767_at | ZNF804A | 91752 |
| 215785_s_at | CYFIP2 | 26999 |
| 215789_s_at | AJAP1 | 55966 |
| 215794_x_at | GLUD2 | 2747 |
| 216594_x_at | AKR1C1 | 1645 |
| 216925_s_at | TAL1 | 6886 |
| 217077_s_at | GABBR2 | 9568 |
| 217359_s_at | NCAM1 | 4684 |
| 217455_s_at | SSTR2 | 6752 |
| 217681_at | WNT7B | 7477 |
| 217897_at | FXYD6 | 53826 |
| 217969_at | C11orf2 | 738 |
| 218228_s_at | TNKS2 | 80351 |
| 218723_s_at | C13orf15 | 28984 |
| 218790_s_at | TMLHE | 55217 |
| 218796_at | FERMT1 | 55612 |
| 218862_at | ASB13 | 79754 |
| 218935_at | EHD3 | 30845 |
| 218938_at | FBXL15 | 79176 |
| 218952_at | PCSK1N | 27344 |
| 218976_at | DNAJC12 | 56521 |
| 219005_at | TMEM59L | 25789 |
| 219093_at | PID1 | 55022 |
| 219107_at | BCAN | 63827 |
| 219144_at | DUSP26 | 78986 |
| 219170_at | FSD1 | 79187 |
| 219196_at | SCG3 | 29106 |
| 219230_at | TMEM100 | 55273 |
| 219273_at | CCNK | 8812 |
| 219305_x_at | FBXO2 | 26232 |
| 219370_at | RPRM | 56475 |
| 219415_at | TTYH1 | 57348 |
| 219521_at | B3GAT1 | 27087 |
| 219537_x_at | DLL3 | 10683 |
| 219732_at | RP11- | 54886 |
| | 35N6.1 | |
| 219743_at | HEY2 | 23493 |
| 219961_s_at | C20orf19 | 55857 |
| 220005_at | P2RY13 | 53829 |
| 220061_at | ACSM5 | 54988 |
| 220188_at | JPH3 | 57338 |
| 221310_at | FGF14 | 2259 |
| 221527_s_at | PARD3 | 56288 |
| 221552_at | ABHD6 | 57406 |
| 221578_at | RASSF4 | 83937 |
| 221623_at | BCAN | 63827 |
| 221679_s_at | ABHD6 | 57406 |
| 221792_at | RAB6B | 51560 |
| 221824_s_at | 9-Mar | 220972 |
| 221861_at | — | — |
| 221959_at | FAM110B | 90362 |
| 222171_s_at | PKNOX2 | 63876 |
| 222783_s_at | SMOC1 | 64093 |
| 222784_at | SMOC1 | 64093 |
| 222898_s_at | DLL3 | 10683 |
| 222957_at | NEU4 | 129807 |
| 223315_at | NTN4 | 59277 |
| 223552_at | LRRC4 | 64101 |
| 223614_at | C8orf57 | 84257 |
| 223839_s_at | SCD | 6319 |
| 223865_at | SOX6 | 55553 |
| 223885_at | CALN1 | 83698 |
| 224215_s_at | DLL1 | 28514 |
| 224393_s_at | CECR6 | 27439 |
| 224482_s_at | RAB11FIP4 | 84440 |
| 224763_at | RPL37 | 6167 |
| 225379_at | MAPT | 4137 |
| 225482_at | KIF1A | 547 |
| 226186_at | TMOD2 | 29767 |
| 226587_at | — | — |
| 226591_at | — | — |
| 226623_at | PHYHIPL | 84457 |
| 226680_at | IKZF5 | 64376 |
| 226913_s_at | SOX8 | 30812 |
| 226918_at | JPH4 | 84502 |
| 227202_at | CNTN1 | 1272 |
| 227341_at | C10orf30 | 222389 |
| 227401_at | IL17D | 53342 |
| 227425_at | REPS2 | 9185 |
| 227440_at | ANKS1B | 56899 |
| 227498_at | — | — |
| 227550_at | LOC143381 | 143381 |
| 227769_at | — | — |
| 227845_s_at | SHD | 56961 |
| 227949_at | PHACTR3 | 116154 |
| 227984_at | LOC650392 | 650392 |
| 228017_s_at | NKAIN4 | 128414 |
| 228018_at | NKAIN4 | 128414 |
| 228051_at | LOC202451 | 202451 |
| 228165_at | C12orf53 | 196500 |
| 228170_at | OLIG1 | 116448 |
| 228193_s_at | C13orf15 | 28984 |
| 228206_at | HS3ST4 | 9951 |
| 228376_at | GGTA1 | 2681 |
| 228403_at | C9orf165 | 375704 |
| 228509_at | SPHKAP | 80309 |
| 228598_at | DPP10 | 57628 |
| 228608_at | NALCN | 259232 |
| 228679_at | — | — |
| 229233_at | NRG3 | 10718 |
| 229234_at | ZC3H12B | 340554 |
| 229294_at | JPH3 | 57338 |
| 229378_at | STOX1 | 219736 |
| 229459_at | FAM19A5 | 25817 |
| 229463_at | NTRK2 | 4915 |
| 229545_at | FERMT1 | 55612 |
| 229590_at | RPL13 | 6137 |
| 229612_at | — | — |
| 229613_at | — | — |
| 229655_at | FAM19A5 | 25817 |
| 229724_at | GABRB3 | 2562 |
| 229799_s_at | NCAM1 | 4684 |
| 229831_at | CNTN3 | 5067 |
| 229875_at | ZDHHC22 | 283576 |
| 229901_at | ZNF488 | 118738 |
| 229921_at | — | — |
| 230287_at | SGSM1 | 129049 |
| 230307_at | SLC25A21 | 89874 |
| 230336_at | — | — |
| 230551_at | KSR2 | 283455 |
| 230568_x_at | DLL3 | 10683 |
| 230577_at | — | — |
| 230771_at | NKAIN4 | 128414 |
| 230869_at | FAM155A | 728215 |
| 230932_at | — | — |
| 230942_at | CMTM5 | 116173 |
| 231103_at | — | — |
| 231131_at | FAM133A | 286499 |
| 231214_at | — | — |
| 231650_s_at | SEZ6L | 23544 |
| 231798_at | NOG | 9241 |
| 231935_at | ARPP-21 | 10777 |
| 231977_at | GRID1 | 2894 |
| 231978_at | TPCN2 | 219931 |
| 231980_at | — | — |
| 232010_at | FSTL5 | 56884 |
| 232059_at | DSCAML1 | 57453 |
| 232192_at | LOC153811 | 153811 |
| 232195_at | GPR158 | 57512 |
| 232833_at | — | — |
| 233051_at | SLITRK2 | 84631 |
| 234472_at | GALNT13 | 114805 |
| 234996_at | CALCRL | 10203 |
| 235118_at | — | — |
| 235527_at | DLGAP1 | 9229 |
| 235591_at | SSTR1 | 6751 |
| 236038_at | — | — |
| 236095_at | NTRK2 | 4915 |
| 236287_at | — | — |
| 236290_at | DOK6 | 220164 |
| 236333_at | — | — |
| 236433_at | — | — |
| 236536_at | GALNT13 | 114805 |
| 236538_at | GRIA2 | 2891 |
| 236576_at | — | — |
| 236748_at | RASGEF1C | 255426 |
| 236771_at | C6orf159 | 134701 |
| 237094_at | FAM19A5 | 25817 |
| 238458_at | EFHA2 | 286097 |
| 238521_at | — | — |
| 238603_at | LOC254559 | 254559 |
| 238663_x_at | GRIA4 | 2893 |
| 239293_at | NRSN1 | 140767 |
| 239509_at | — | — |
| 239787_at | KCTD4 | 386618 |
| 239827_at | C13orf15 | 28984 |
| 240067_at | — | — |
| 240218_at | DSCAM | 1826 |
| 240228_at | CSMD3 | 114788 |
| 240433_x_at | — | — |
| 240512_x_at | KCTD4 | 386618 |
| 240578_at | — | — |
| 240869_at | — | — |
| 241255_at | — | — |
| 241365_at | — | — |
| 241729_at | DOK6 | 220164 |
| 241909_at | TNKS2 | 80351 |
| 242571_at | REPS2 | 9185 |
| 242651_at | — | — |
| 243526_at | WDR86 | 349136 |
| 243779_at | GALNT13 | 114805 |
| 243952_at | psiTPTE22 | 387590 |
| 244184_at | — | — |
| 244218_at | — | — |
| 244623_at | KCNQ5 | 56479 |
| 35846_at | THRA | 7067 |
| 43511_s_at | — | — |
| 49111_at | — | — |
| 60474_at | FERMT1 | 55612 |
| 89977_at | ACSM5 | 54988 |
| 91920_at | BCAN | 63827 |

TABLE 3C
Table 3C. Genes in the PROGES signature.

| AffyID | Gene Symbol | Gene ID |
|---|---|---|
| 200934_at | DEK | 7913 |
| 201016_at | EIF1AX | 1964 |
| 201202_at | PCNA | 5111 |
| 201291_s_at | TOP2A | 7153 |
| 201292_at | TOP2A | 7153 |
| 201477_s_at | RRM1 | 6240 |
| 201663_s_at | SMC4 | 10051 |
| 201664_at | SMC4 | 10051 |
| 201764_at | TMEM106C | 79022 |
| 201890_at | RRM2 | 6241 |
| 201930_at | MCM6 | 4175 |
| 201970_s_at | NASP | 4678 |
| 202107_s_at | MCM2 | 4171 |
| 202276_at | SHFM1 | 7979 |
| 202412_s_at | USP1 | 7398 |
| 202503_s_at | KIAA0101 | 9768 |
| 202532_s_at | DHFR | 1719 |
| 202533_s_at | DHFR | 1719 |
| 202534_x_at | DHFR | 1719 |
| 202589_at | TYMS | 7298 |
| 202904_s_at | LSM5 | 23658 |
| 202979_s_at | CREBZF | 58487 |
| 203046_s_at | TIMELESS | 8914 |
| 203213_at | CDC2 | 983 |
| 203276_at | LMNB1 | 4001 |
| 203344_s_at | RBBP8 | 5932 |
| 203347_s_at | MTF2 | 22823 |
| 203358_s_at | EZH2 | 2146 |
| 203362_s_at | MAD2L1 | 4085 |
| 203401_at | PRPS2 | 5634 |
| 203560_at | GGH | 8836 |
| 203675_at | NUCB2 | 4925 |
| 203764_at | DLGAP5 | 9787 |
| 203830_at | C17orf75 | 64149 |
| 203925_at | GCLM | 2730 |
| 203960_s_at | HSPB11 | 51668 |
| 203967_at | CDC6 | 990 |
| 203968_s_at | CDC6 | 990 |
| 203976_s_at | CHAF1A | 10036 |
| 204005_s_at | PAWR | 5074 |
| 204023_at | RFC4 | 5984 |
| 204026_s_at | ZWINT | 11130 |
| 204092_s_at | AURKA | 6790 |
| 204146_at | RAD51AP1 | 10635 |
| 204159_at | CDKN2C | 1031 |
| 204162_at | NDC80 | 10403 |
| 204170_s_at | CKS2 | 1164 |
| 204240_s_at | SMC2 | 10592 |
| 204244_s_at | DBF4 | 10926 |
| 204252_at | CDK2 | 1017 |
| 204342_at | SLC25A24 | 29957 |
| 204485_s_at | TOM1L1 | 10040 |
| 204517_at | PPIC | 5480 |
| 204531_s_at | BRCA1 | 672 |
| 204641_at | NEK2 | 4751 |
| 204709_s_at | KIF23 | 9493 |
| 204775_at | CHAF1B | 8208 |
| 204784_s_at | MLF1 | 4291 |
| 204822_at | TTK | 7272 |
| 204825_at | MELK | 9833 |
| 204833_at | ATG12 | 9140 |
| 204886_at | PLK4 | 10733 |
| 204947_at | E2F1 | 1869 |
| 204962_s_at | CENPA | 1058 |
| 205023_at | RAD51 | 5888 |
| 205034_at | CCNE2 | 9134 |
| 205046_at | CENPE | 1062 |
| 205061_s_at | EXOSC9 | 5393 |
| 205063_at | SIP1 | 8487 |
| 205071_x_at | XRCC4 | 7518 |
| 205167_s_at | CDC25C | 995 |
| 205176_s_at | ITGB3BP | 23421 |
| 205260_s_at | ACYP1 | 97 |
| 205339_at | STIL | 6491 |
| 205345_at | BARD1 | 580 |
| 205393_s_at | CHEK1 | 1111 |
| 205394_at | CHEK1 | 1111 |
| 205628_at | PRIM2 | 5558 |
| 206102_at | GINS1 | 9837 |
| 206172_at | IL13RA2 | 3598 |
| 206316_s_at | KNTC1 | 9735 |
| 206364_at | KIF14 | 9928 |
| 207039_at | CDKN2A | 1029 |
| 207165_at | HMMR | 3161 |
| 208051_s_at | PAIP1 | 10605 |
| 208079_s_at | AURKA | 6790 |
| 208443_x_at | SHOX2 | 6474 |
| 208808_s_at | HMGB2 | 3148 |
| 208995_s_at | PPIG | 9360 |
| 209172_s_at | CENPF | 1063 |
| 209507_at | RPA3 | 6119 |
| 209642_at | BUB1 | 699 |
| 209644_x_at | CDKN2A | 1029 |
| 209709_s_at | HMMR | 3161 |
| 210093_s_at | MAGOH | 4116 |
| 210691_s_at | CACYBP | 27101 |
| 211200_s_at | EFCAB2 | 84288 |
| 211675_s_at | MDFIC | 29969 |
| 211713_x_at | KIAA0101 | 9768 |
| 211747_s_at | LSM5 | 23658 |
| 212094_at | PEG10 | 23089 |
| 212533_at | WEE1 | 7465 |
| 212918_at | RECQL | 5965 |
| 212949_at | NCAPH | 23397 |
| 213007_at | FANCI | 55215 |
| 213017_at | ABHD3 | 171586 |
| 213226_at | CCNA2 | 890 |
| 213253_at | SMC2 | 10592 |
| 213353_at | ABCA5 | 23461 |
| 213424_at | KIAA0895 | 23366 |
| 214224_s_at | PIN4 | 5303 |
| 214431_at | GMPS | 8833 |
| 214710_s_at | CCNB1 | 891 |
| 214804_at | CENPI | 2491 |
| 216228_s_at | WDHD1 | 11169 |
| 218349_s_at | ZWILCH | 55055 |
| 218355_at | KIF4A | 24137 |
| 218585_s_at | DTL | 51514 |
| 218602_s_at | FAM29A | 54801 |
| 218662_s_at | NCAPG | 64151 |
| 218663_at | NCAPG | 64151 |
| 218726_at | HJURP | 55355 |
| 218772_x_at | TMEM38B | 55151 |
| 218875_s_at | FBXO5 | 26271 |
| 218883_s_at | MLF1IP | 79682 |
| 218894_s_at | MAGOHB | 55110 |
| 218911_at | YEATS4 | 8089 |
| 218981_at | ACN9 | 57001 |
| 219105_x_at | ORC6L | 23594 |
| 219174_at | IFT74 | 80173 |
| 219208_at | FBXO11 | 80204 |
| 219288_at | C3orf14 | 57415 |
| 219512_at | DSN1 | 79980 |
| 219555_s_at | CENPN | 55839 |
| 219587_at | TTC12 | 54970 |
| 219650_at | ERCC6L | 54821 |
| 219703_at | MNS1 | 55329 |
| 219736_at | TRIM36 | 55521 |
| 219758_at | TTC26 | 79989 |
| 219787_s_at | ECT2 | 1894 |
| 219918_s_at | ASPM | 259266 |
| 219978_s_at | NUSAP1 | 51203 |
| 219990_at | E2F8 | 79733 |
| 220060_s_at | C12orf48 | 55010 |
| 220144_s_at | ANKRD5 | 63926 |
| 220175_s_at | CBWD1 | 55871 |
| 220840_s_at | C1orf112 | 55732 |
| 221258_s_at | KIF18A | 81930 |
| 221521_s_at | GINS2 | 51659 |
| 221677_s_at | DONSON | 29980 |
| 222606_at | ZWILCH | 55055 |
| 222768_s_at | TRMT6 | 51605 |
| 222848_at | CENPK | 64105 |
| 223133_at | TMEM14B | 81853 |
| 223274_at | TCF19 | 6941 |
| 223381_at | NUF2 | 83540 |
| 223542_at | ANKRD32 | 84250 |
| 223544_at | TMEM79 | 84283 |
| 223700_at | MND1 | 84057 |
| 224204_x_at | ARNTL2 | 56938 |
| 224428_s_at | CDCA7 | 83879 |
| 224443_at | C1orf97 | 84791 |
| 224444_s_at | C1orf97 | 84791 |
| 224715_at | WDR34 | 89891 |
| 224944_at | TMPO | 7112 |
| 225078_at | EMP2 | 2013 |
| 225297_at | CCDC5 | 115106 |
| 226117_at | TIFA | 92610 |
| 226223_at | PAWR | 5074 |
| 226231_at | PAWR | 5074 |
| 226287_at | CCDC34 | 91057 |
| 226452_at | PDK1 | 5163 |
| 226908_at | LRIG3 | 121227 |
| 226936_at | C6orf173 | 387103 |
| 227314_at | ITGA2 | 3673 |
| 227350_at | HELLS | 3070 |
| 227793_at | — | — |
| 228033_at | E2F7 | 144455 |
| 228280_at | ZC3HAV1L | 92092 |
| 228654_at | SPIN4 | 139886 |
| 228729_at | CCNB1 | 891 |
| 228776_at | GJC1 | 10052 |
| 229305_at | MLF1IP | 79682 |
| 229490_s_at | — | — |
| 229551_x_at | ZNF367 | 195828 |
| 229974_at | EVC2 | 132884 |
| 230121_at | C1orf133 | 574036 |
| 230165_at | SGOL2 | 151246 |
| 230696_at | — | — |
| 230860_at | C3orf34 | 84984 |
| 232065_x_at | CENPL | 91687 |
| 232242_at | — | — |
| 233970_s_at | TRMT6 | 51605 |
| 234863_x_at | FBXO5 | 26271 |
| 235004_at | RBM24 | 221662 |
| 235113_at | PPIL5 | 122769 |
| 235425_at | SGOL2 | 151246 |
| 235572_at | SPC24 | 147841 |
| 235609_at | — | — |
| 235644_at | CCDC138 | 165055 |
| 235949_at | — | — |
| 236222_at | C3orf15 | 89876 |
| 236641_at | KIF14 | 9928 |
| 236915_at | C4orf47 | 441054 |
| 237469_at | TOP2A | 7153 |
| 237585_at | C4orf47 | 441054 |
| 238021_s_at | hCG_1815491 | 643911 |
| 238022_at | hCG_1815491 | 643911 |
| 238075_at | — | — |
| 238843_at | NPHP1 | 4867 |
| 238865_at | PABPC4L | 132430 |
| 239413_at | CEP152 | 22995 |
| 239680_at | — | — |
| 241705_at | ABCA5 | 23461 |
| 242560_at | FANCD2 | 2177 |
| 243198_at | TEX9 | 374618 |
| 48808_at | DHFR | 1719 |

TABLE 4
List of 928 Transcription Factors used by the MRA analysis.

| No. | TF Name |
|---|---|
| 1 | AATF |
| 2 | ADNP |
| 3 | AEBP1 |
| 4 | AFF1 |
| 5 | AFF3 |
| 6 | AFF4 |
| 7 | AHCTF1 |
| 8 | AHR |
| 9 | ALX4 |
| 10 | AR |
| 11 | ARID3A |
| 12 | ARID4A |
| 13 | ARNT |
| 14 | ARNT2 |
| 15 | ARNTL |
| 16 | ARNTL2 |
| 17 | ASCL1 |
| 18 | ASCL2 |
| 19 | ATBF1 |
| 20 | ATF1 |
| 21 | ATF2 |
| 22 | ATF3 |
| 23 | ATF4 |
| 24 | ATF5 |
| 25 | ATF6 |
| 26 | ATF7 |
| 27 | ATOH1 |
| 28 | BACH1 |
| 29 | BACH2 |
| 30 | BAPX1 |
| 31 | BARX2 |
| 32 | BATF |
| 33 | BAZ1B |
| 34 | BCL6 |
| 35 | BHLHB2 |
| 36 | BHLHB3 |
| 37 | BLZF1 |
| 38 | BNC1 |
| 39 | BRD8 |
| 40 | BRF1 |
| 41 | BRPF1 |
| 42 | BTAF1 |
| 43 | BUD31 |
| 44 | C2orf3 |
| 45 | CBFA2T2 |
| 46 | CBFA2T3 |
| 47 | CBFB |
| 48 | CBL |
| 49 | CCRN4L |
| 50 | CDX1 |
| 51 | CDX2 |
| 52 | CDX4 |
| 53 | CEBPA |
| 54 | CEBPB |
| 55 | CEBPD |
| 56 | CEBPE |
| 57 | CEBPG |
| 58 | CEBPZ |
| 59 | CHES1 |
| 60 | CIITA |
| 61 | CIR |
| 62 | CITED1 |
| 63 | CITED2 |
| 64 | CLOCK |
| 65 | CNBP |
| 66 | CNOT7 |
| 67 | CNOT8 |
| 68 | CREB1 |
| 69 | CREB3 |
| 70 | CREB3L1 |
| 71 | CREB3L2 |
| 72 | CREB5 |
| 73 | CREBBP |
| 74 | CREBL1 |
| 75 | CREBL2 |
| 76 | CREG1 |
| 77 | CREM |
| 78 | CRX |
| 79 | CSDA |
| 80 | CTBP1 |
| 81 | CTBP2 |
| 82 | CTCF |
| 83 | CTNNB1 |
| 84 | CUTL1 |
| 85 | CUTL2 |
| 86 | DAXX |
| 87 | DBP |
| 88 | DDIT3 |
| 89 | DEK |
| 90 | DENND4A |
| 91 | DLX2 |
| 92 | DLX4 |
| 93 | DLX5 |
| 94 | DLX6 |
| 95 | DMTF1 |
| 96 | DR1 |
| 97 | DRAP1 |
| 98 | DSCR1 |
| 99 | DUX1 |
| 100 | E2F1 |
| 101 | E2F2 |
| 102 | E2F3 |
| 103 | E2F4 |
| 104 | E2F5 |
| 105 | E2F6 |
| 106 | E2F8 |
| 107 | E4F1 |
| 108 | EDF1 |
| 109 | EGR1 |
| 110 | EGR2 |
| 111 | EGR3 |
| 112 | EGR4 |
| 113 | ELF1 |
| 114 | ELF2 |
| 115 | ELF3 |
| 116 | ELF4 |
| 117 | ELF5 |
| 118 | ELK1 |
| 119 | ELK3 |
| 120 | ELK4 |
| 121 | EMX1 |
| 122 | EMX2 |
| 123 | EN1 |
| 124 | EN2 |
| 125 | ENO1 |
| 126 | EP300 |
| 127 | EPAS1 |
| 128 | ERCC6 |
| 129 | ERF |
| 130 | ERG |
| 131 | ESR1 |
| 132 | ESR2 |
| 133 | ESRRA |
| 134 | ESRRB |
| 135 | ESRRG |
| 136 | ETS1 |
| 137 | ETS2 |
| 138 | ETV1 |
| 139 | ETV3 |
| 140 | ETV4 |
| 141 | ETV5 |
| 142 | ETV6 |
| 143 | ETV7 |
| 144 | EVI1 |
| 145 | EVX1 |
| 146 | EWSR1 |
| 147 | FALZ |
| 148 | FEV |
| 149 | FEZF2 |
| 150 | FLI1 |
| 151 | FMNL2 |
| 152 | FOS |
| 153 | FOSB |
| 154 | FOSL1 |
| 155 | FOSL2 |
| 156 | FOXA1 |
| 157 | FOXA2 |
| 158 | FOXB1 |
| 159 | FOXD1 |
| 160 | FOXD3 |
| 161 | FOXE1 |
| 162 | FOXE3 |
| 163 | FOXF1 |
| 164 | FOXF2 |
| 165 | FOXG1B |
| 166 | FOXH1 |
| 167 | FOXI1 |
| 168 | FOXJ1 |
| 169 | FOXJ2 |
| 170 | FOXJ3 |
| 171 | FOXK2 |
| 172 | FOXL1 |
| 173 | FOXM1 |
| 174 | FOXN1 |
| 175 | FOXO1A |
| 176 | FOXO3A |
| 177 | FOXP1 |
| 178 | FOXP3 |
| 179 | FUBP1 |
| 180 | FUBP3 |
| 181 | GABPB2 |
| 182 | GAS7 |
| 183 | GATA1 |
| 184 | GATA2 |
| 185 | GATA3 |
| 186 | GATA4 |
| 187 | GATA6 |
| 188 | GATAD1 |
| 189 | GATAD2A |
| 190 | GBX2 |
| 191 | GLI2 |
| 192 | GLI3 |
| 193 | GMEB1 |
| 194 | GRLF1 |
| 195 | GTF2IRD1 |
| 196 | HAND1 |
| 197 | HAND2 |
| 198 | HBP1 |
| 199 | HCFC1 |
| 200 | HCLS1 |
| 201 | HES1 |
| 202 | HES2 |
| 203 | HESX1 |
| 204 | HEY1 |
| 205 | HEY2 |
| 206 | HEYL |
| 207 | HHEX |
| 208 | HIC1 |
| 209 | HIF1A |
| 210 | HIF3A |
| 211 | HIRA |
| 212 | HIVEP1 |
| 213 | HIVEP2 |
| 214 | HIVEP3 |
| 215 | HKR3 |
| 216 | HLF |
| 217 | HLX1 |
| 218 | HLXB9 |
| 219 | HMBOX1 |
| 220 | HMG20A |
| 221 | HMG20B |
| 222 | HMGA1 |
| 223 | HMGA2 |
| 224 | HMGB1 |
| 225 | HMGB2 |
| 226 | HMX1 |
| 227 | HNF4A |
| 228 | HNF4G |
| 229 | HOP |
| 230 | HOXA1 |
| 231 | HOXA10 |
| 232 | HOXA11 |
| 233 | HOXA2 |
| 234 | HOXA3 |
| 235 | HOXA4 |
| 236 | HOXA5 |
| 237 | HOXA6 |
| 238 | HOXA7 |
| 239 | HOXA9 |
| 240 | HOXB13 |
| 241 | HOXB2 |
| 242 | HOXB5 |
| 243 | HOXB6 |
| 244 | HOXB7 |
| 245 | HOXB8 |
| 246 | HOXB9 |
| 247 | HOXC10 |
| 248 | HOXC11 |
| 249 | HOXC4 |
| 250 | HOXC5 |
| 251 | HOXC6 |
| 252 | HOXD1 |
| 253 | HOXD10 |
| 254 | HOXD11 |
| 255 | HOXD12 |
| 256 | HOXD13 |
| 257 | HOXD3 |
| 258 | HOXD4 |
| 259 | HOXD9 |
| 260 | H-plk |
| 261 | HR |
| 262 | HSF1 |
| 263 | HSF2 |
| 264 | HSF4 |
| 265 | HTLF |
| 266 | IKZF1 |
| 267 | IKZF4 |
| 268 | IKZF5 |
| 269 | ILF2 |
| 270 | INSM1 |
| 271 | IPF1 |
| 272 | IRF1 |
| 273 | IRF2 |
| 274 | IRF3 |
| 275 | IRF4 |
| 276 | IRF5 |
| 277 | IRF6 |
| 278 | IRF7 |
| 279 | IRF8 |
| 280 | IRX4 |
| 281 | IRX5 |
| 282 | ISGF3G |
| 283 | ISL1 |
| 284 | JARID1A |
| 285 | JARID1B |
| 286 | JUN |
| 287 | JUNB |
| 288 | JUND |
| 289 | KIAA0415 |
| 290 | KIAA0963 |
| 291 | KLF1 |
| 292 | KLF10 |
| 293 | KLF11 |
| 294 | KLF12 |
| 295 | KLF13 |
| 296 | KLF15 |
| 297 | KLF2 |
| 298 | KLF3 |
| 299 | KLF4 |
| 300 | KLF5 |
| 301 | KLF6 |
| 302 | KLF7 |
| 303 | KLF9 |
| 304 | KNTC1 |
| 305 | L3MBTL |
| 306 | LASS2 |
| 307 | LASS4 |
| 308 | LASS6 |
| 309 | LBX1 |
| 310 | LHX2 |
| 311 | LHX3 |
| 312 | LHX5 |
| 313 | LHX6 |
| 314 | LMO1 |
| 315 | LMO4 |
| 316 | LMX1B |
| 317 | LOC645682 |
| 318 | LYL1 |
| 319 | LZTFL1 |
| 320 | LZTR1 |
| 321 | LZTS1 |
| 322 | MAF |
| 323 | MAFB |
| 324 | MAFF |
| 325 | MAFG |
| 326 | MAFK |
| 327 | MAML3 |
| 328 | MAX |
| 329 | MAZ |
| 330 | MBD1 |
| 331 | MDS1 |
| 332 | MECP2 |
| 333 | MEF2A |
| 334 | MEF2B |
| 335 | MEF2C |
| 336 | MEF2D |
| 337 | MEIS1 |
| 338 | MEIS2 |
| 339 | MEIS3P1 |
| 340 | MEOX1 |
| 341 | MEOX2 |
| 342 | MGA |
| 343 | MITF |
| 344 | MIZF |
| 345 | MLL |
| 346 | MLL4 |
| 347 | MLLT10 |
| 348 | MLLT7 |
| 349 | MLX |
| 350 | MLXIP |
| 351 | MLXIPL |
| 352 | MNT |
| 353 | MSC |
| 354 | MSL3L1 |
| 355 | MSRB2 |
| 356 | MSX1 |
| 357 | MSX2 |
| 358 | MTA1 |
| 359 | MTA2 |
| 360 | MTF1 |
| 361 | MXD1 |
| 362 | MYB |
| 363 | MYBL1 |
| 364 | MYBL2 |
| 365 | MYC |
| 366 | MYCL1 |
| 367 | MYCN |
| 368 | MYF6 |
| 369 | MYNN |
| 370 | MYOD1 |
| 371 | MYOG |
| 372 | MYST2 |
| 373 | MYT1 |
| 374 | MYT1L |
| 375 | MZF1 |
| 376 | NANOG |
| 377 | NCOR1 |
| 378 | NEUROD1 |
| 379 | NEUROD2 |
| 380 | NEUROG1 |
| 381 | NEUROG3 |
| 382 | NFAT5 |
| 383 | NFATC1 |
| 384 | NFATC3 |
| 385 | NFATC4 |
| 386 | NFE2 |
| 387 | NFE2L1 |
| 388 | NFE2L2 |
| 389 | NFE2L3 |
| 390 | NFIB |
| 391 | NFIC |
| 392 | NFIL3 |
| 393 | NFIX |
| 394 | NFKB1 |
| 395 | NFKB2 |
| 396 | NFRKB |
| 397 | NFX1 |
| 398 | NFYA |
| 399 | NFYB |
| 400 | NFYC |
| 401 | NHLH1 |
| 402 | NHLH2 |
| 403 | NKRF |
| 404 | NKX2-2 |
| 405 | NKX2-5 |
| 406 | NKX2-8 |
| 407 | NKX3-1 |
| 408 | NKX6-1 |
| 409 | NOTCH2 |
| 410 | NPAS2 |
| 411 | NPAS3 |
| 412 | NPAT |
| 413 | NR0B1 |
| 414 | NR0B2 |
| 415 | NR1D2 |
| 416 | NR1H2 |
| 417 | NR1H3 |
| 418 | NR1H4 |
| 419 | NR1I2 |
| 420 | NR1I3 |
| 421 | NR2C1 |
| 422 | NR2C2 |
| 423 | NR2E1 |
| 424 | NR2E3 |
| 425 | NR2F1 |
| 426 | NR2F2 |
| 427 | NR2F6 |
| 428 | NR3C1 |
| 429 | NR3C2 |
| 430 | NR4A1 |
| 431 | NR4A2 |
| 432 | NR4A3 |
| 433 | NR5A1 |
| 434 | NR5A2 |
| 435 | NR6A1 |
| 436 | NRF1 |
| 437 | NRL |
| 438 | OLIG2 |
| 439 | ONECUT1 |
| 440 | OVOL1 |
| 441 | PAX1 |
| 442 | PAX2 |
| 443 | PAX3 |
| 444 | PAX4 |
| 445 | PAX6 |
| 446 | PAX7 |
| 447 | PAX8 |
| 448 | PAX9 |
| 449 | PBX1 |
| 450 | PBX2 |
| 451 | PBX3 |
| 452 | PCGF2 |
| 453 | PEG3 |
| 454 | PFDN1 |
| 455 | PGR |
| 456 | PHF2 |
| 457 | PHOX2A |
| 458 | PHOX2B |
| 459 | PHTF1 |
| 460 | PHTF2 |
| 461 | PITX1 |
| 462 | PITX3 |
| 463 | PKNOX1 |
| 464 | PKNOX2 |
| 465 | PLAG1 |
| 466 | PLAGL1 |
| 467 | PLAGL2 |
| 468 | PML |
| 469 | POU2F1 |
| 470 | POU2F2 |
| 471 | POU2F3 |
| 472 | POU3F1 |
| 473 | POU3F2 |
| 474 | POU3F3 |
| 475 | POU3F4 |
| 476 | POU4F1 |
| 477 | POU4F2 |
| 478 | POU6F1 |
| 479 | POU6F2 |
| 480 | PPARA |
| 481 | PPARD |
| 482 | PPARG |
| 483 | PRDM1 |
| 484 | PRDM16 |
| 485 | PRDM2 |
| 486 | PREB |
| 487 | PROP1 |
| 488 | PRRX1 |
| 489 | PRRX2 |
| 490 | PTTG1 |
| 491 | PURA |
| 492 | RARA |
| 493 | RARB |
| 494 | RARG |
| 495 | RAX |
| 496 | RB1 |
| 497 | RBL2 |
| 498 | RBPSUH |
| 499 | RBPSUHL |
| 500 | REL |
| 501 | RELA |
| 502 | RELB |
| 503 | RERE |
| 504 | REST |
| 505 | REXO4 |
| 506 | RFX1 |
| 507 | RFX2 |
| 508 | RFX3 |
| 509 | RFX5 |
| 510 | RFXANK |
| 511 | RFXAP |
| 512 | RLF |
| 513 | RNF4 |
| 514 | RORA |
| 515 | RORB |
| 516 | RORC |
| 517 | RREB1 |
| 518 | RUNX1 |
| 519 | RUNX1T1 |
| 520 | RUNX2 |
| 521 | RUNX3 |
| 522 | RXRA |
| 523 | RXRB |
| 524 | RXRG |
| 525 | SALL1 |
| 526 | SALL2 |
| 527 | SATB1 |
| 528 | SATB2 |
| 529 | SCAND1 |
| 530 | SCAND2 |
| 531 | SCML1 |
| 532 | SCML2 |
| 533 | SHOX |
| 534 | SHOX2 |
| 535 | SIM2 |
| 536 | SIX1 |
| 537 | SIX2 |
| 538 | SIX3 |
| 539 | SIX5 |
| 540 | SIX6 |
| 541 | SLC26A3 |
| 542 | SLC2A4RG |
| 543 | SLC30A9 |
| 544 | SMAD1 |
| 545 | SMAD2 |
| 546 | SMAD3 |
| 547 | SMAD4 |
| 548 | SMAD5 |
| 549 | SMAD6 |
| 550 | SMAD7 |
| 551 | SMAD9 |
| 552 | SMARCA3 |
| 553 | SMARCA4 |
| 554 | SNAI1 |
| 555 | SNAI2 |
| 556 | SNAPC2 |
| 557 | SNAPC4 |
| 558 | SNAPC5 |
| 559 | SNFT |
| 560 | SOLH |
| 561 | SOX1 |
| 562 | SOX10 |
| 563 | SOX11 |
| 564 | SOX12 |
| 565 | SOX13 |
| 566 | SOX15 |
| 567 | SOX17 |
| 568 | SOX18 |
| 569 | SOX2 |
| 570 | SOX21 |
| 571 | SOX3 |
| 572 | SOX4 |
| 573 | SOX5 |
| 574 | SOX9 |
| 575 | SP1 |
| 576 | SP140 |
| 577 | SP2 |
| 578 | SP3 |
| 579 | SP4 |
| 580 | SPDEF |
| 581 | SPI1 |
| 582 | SPIB |
| 583 | SREBF1 |
| 584 | SREBF2 |
| 585 | SRF |
| 586 | ST18 |
| 587 | STAT1 |
| 588 | STAT2 |
| 589 | STAT3 |
| 590 | STAT4 |
| 591 | STAT5A |
| 592 | STAT5B |
| 593 | STAT6 |
| 594 | SUPT4H1 |
| 595 | SUPT6H |
| 596 | T |
| 597 | TADA2L |
| 598 | TADA3L |
| 599 | TAF1B |
| 600 | TAF5L |
| 601 | TAL1 |
| 602 | TARDBP |
| 603 | TBR1 |
| 604 | TBX1 |
| 605 | TBX10 |
| 606 | TBX19 |
| 607 | TBX2 |
| 608 | TBX21 |
| 609 | TBX3 |
| 610 | TBX4 |
| 611 | TBX5 |
| 612 | TBX6 |
| 613 | TCEAL1 |
| 614 | TCF1 |
| 615 | TCF12 |
| 616 | TCF15 |
| 617 | TCF2 |
| 618 | TCF21 |
| 619 | TCF25 |
| 620 | TCF3 |
| 621 | TCF4 |
| 622 | TCF7 |
| 623 | TCF7L1 |
| 624 | TCF7L2 |
| 625 | TCF8 |
| 626 | TCFL5 |
| 627 | TEAD1 |
| 628 | TEAD3 |
| 629 | TEAD4 |
| 630 | TEF |
| 631 | TFAM |
| 632 | TFAP2A |
| 633 | TFAP2B |
| 634 | TFAP2C |
| 635 | TFAP4 |
| 636 | TFCP2 |
| 637 | TFCP2L1 |
| 638 | TFDP1 |
| 639 | TFDP2 |
| 640 | TFDP3 |
| 641 | TFE3 |
| 642 | TFEB |
| 643 | TFEC |
| 644 | TGIF |
| 645 | TGIF2 |
| 646 | THRA |
| 647 | THRB |
| 648 | TLX1 |
| 649 | TLX2 |
| 650 | TNRC4 |
| 651 | TP53 |
| 652 | TP73 |
| 653 | TP73L |
| 654 | TRERF1 |
| 655 | TRIM22 |
| 656 | TRIM25 |
| 657 | TRIM28 |
| 658 | TRIM29 |
| 659 | TRPS1 |
| 660 | TSC22D1 |
| 661 | TSC22D2 |
| 662 | TSC22D3 |
| 663 | TSC22D4 |
| 664 | TULP4 |
| 665 | TWIST1 |
| 666 | UBN1 |
| 667 | UBP1 |
| 668 | USF2 |
| 669 | VAV1 |
| 670 | VAX2 |
| 671 | VDR |
| 672 | VENTX |
| 673 | VEZF1 |
| 674 | VPS72 |
| 675 | VSX1 |
| 676 | WT1 |
| 677 | XBP1 |
| 678 | YBX1 |
| 679 | YEATS4 |
| 680 | YWHAE |
| 681 | YWHAZ |
| 682 | YY1 |
| 683 | YY2 |
| 684 | ZBTB16 |
| 685 | ZBTB17 |
| 686 | ZBTB22 |
| 687 | ZBTB25 |
| 688 | ZBTB38 |
| 689 | ZBTB43 |
| 690 | ZBTB6 |
| 691 | ZBTB7A |
| 692 | ZBTB7B |
| 693 | ZF |
| 694 | ZFHX1B |
| 695 | ZFHX4 |
| 696 | ZFP36L1 |
| 697 | ZFP36L2 |
| 698 | ZFP37 |
| 699 | ZFP95 |
| 700 | ZFX |
| 701 | ZFY |
| 702 | ZHX2 |
| 703 | ZHX3 |
| 704 | ZIC1 |
| 705 | ZIM2 |
| 706 | ZKSCAN1 |
| 707 | ZMYM2 |
| 708 | ZMYM3 |
| 709 | ZMYM4 |
| 710 | ZNF10 |
| 711 | ZNF117 |
| 712 | ZNF12 |
| 713 | ZNF124 |
| 714 | ZNF131 |
| 715 | ZNF132 |
| 716 | ZNF133 |
| 717 | ZNF134 |
| 718 | ZNF135 |
| 719 | ZNF136 |
| 720 | ZNF137 |
| 721 | ZNF14 |
| 722 | ZNF140 |
| 723 | ZNF141 |
| 724 | ZNF142 |
| 725 | ZNF143 |
| 726 | ZNF146 |
| 727 | ZNF148 |
| 728 | ZNF154 |
| 729 | ZNF155 |
| 730 | ZNF16 |
| 731 | ZNF160 |
| 732 | ZNF167 |
| 733 | ZNF174 |
| 734 | ZNF175 |
| 735 | ZNF177 |
| 736 | ZNF180 |
| 737 | ZNF184 |
| 738 | ZNF185 |
| 739 | ZNF187 |
| 740 | ZNF189 |
| 741 | ZNF192 |
| 742 | ZNF193 |
| 743 | ZNF195 |
| 744 | ZNF197 |
| 745 | ZNF20 |
| 746 | ZNF200 |
| 747 | ZNF202 |
| 748 | ZNF204 |
| 749 | ZNF205 |
| 750 | ZNF207 |
| 751 | ZNF211 |
| 752 | ZNF212 |
| 753 | ZNF215 |
| 754 | ZNF217 |
| 755 | ZNF219 |
| 756 | ZNF22 |
| 757 | ZNF221 |
| 758 | ZNF222 |
| 759 | ZNF223 |
| 760 | ZNF224 |
| 761 | ZNF225 |
| 762 | ZNF226 |
| 763 | ZNF227 |
| 764 | ZNF228 |
| 765 | ZNF230 |
| 766 | ZNF232 |
| 767 | ZNF235 |
| 768 | ZNF236 |
| 769 | ZNF238 |
| 770 | ZNF239 |
| 771 | ZNF24 |
| 772 | ZNF248 |
| 773 | ZNF250 |
| 774 | ZNF253 |
| 775 | ZNF259 |
| 776 | ZNF26 |
| 777 | ZNF263 |
| 778 | ZNF264 |
| 779 | ZNF266 |
| 780 | ZNF267 |
| 781 | ZNF268 |
| 782 | ZNF271 |
| 783 | ZNF273 |
| 784 | ZNF274 |
| 785 | ZNF277 |
| 786 | ZNF278 |
| 787 | ZNF281 |
| 788 | ZNF282 |
| 789 | ZNF286 |
| 790 | ZNF287 |
| 791 | ZNF289 |
| 792 | ZNF291 |
| 793 | ZNF292 |
| 794 | ZNF294 |
| 795 | ZNF3 |
| 796 | ZNF302 |
| 797 | ZNF304 |
| 798 | ZNF306 |
| 799 | ZNF307 |
| 800 | ZNF313 |
| 801 | ZNF318 |
| 802 | ZNF32 |
| 803 | ZNF322B |
| 804 | ZNF323 |
| 805 | ZNF324 |
| 806 | ZNF329 |
| 807 | ZNF330 |
| 808 | ZNF331 |
| 809 | ZNF334 |
| 810 | ZNF335 |
| 811 | ZNF337 |
| 812 | ZNF33B |
| 813 | ZNF34 |
| 814 | ZNF343 |
| 815 | ZNF345 |
| 816 | ZNF35 |
| 817 | ZNF350 |
| 818 | ZNF354A |
| 819 | ZNF358 |
| 820 | ZNF364 |
| 821 | ZNF365 |
| 822 | ZNF384 |
| 823 | ZNF394 |
| 824 | ZNF395 |
| 825 | ZNF403 |
| 826 | ZNF407 |
| 827 | ZNF408 |
| 828 | ZNF409 |
| 829 | ZNF410 |
| 830 | ZNF415 |
| 831 | ZNF419A |
| 832 | ZNF42 |
| 833 | ZNF423 |
| 834 | ZNF426 |
| 835 | ZNF43 |
| 836 | ZNF430 |
| 837 | ZNF432 |
| 838 | ZNF434 |
| 839 | ZNF435 |
| 840 | ZNF44 |
| 841 | ZNF440 |
| 842 | ZNF443 |
| 843 | ZNF444 |
| 844 | ZNF446 |
| 845 | ZNF447 |
| 846 | ZNF45 |
| 847 | ZNF451 |
| 848 | ZNF460 |
| 849 | ZNF467 |
| 850 | ZNF468 |
| 851 | ZNF471 |
| 852 | ZNF473 |
| 853 | ZNF480 |
| 854 | ZNF484 |
| 855 | ZNF493 |
| 856 | ZNF500 |
| 857 | ZNF506 |
| 858 | ZNF507 |
| 859 | ZNF508 |
| 860 | ZNF510 |
| 861 | ZNF516 |
| 862 | ZNF518 |
| 863 | ZNF528 |
| 864 | ZNF529 |
| 865 | ZNF532 |
| 866 | ZNF536 |
| 867 | ZNF544 |
| 868 | ZNF549 |
| 869 | ZNF550 |
| 870 | ZNF551 |
| 871 | ZNF552 |
| 872 | ZNF556 |
| 873 | ZNF557 |
| 874 | ZNF562 |
| 875 | ZNF573 |
| 876 | ZNF574 |
| 877 | ZNF576 |
| 878 | ZNF580 |
| 879 | ZNF586 |
| 880 | ZNF587 |
| 881 | ZNF588 |
| 882 | ZNF589 |
| 883 | ZNF592 |
| 884 | ZNF593 |
| 885 | ZNF606 |
| 886 | ZNF609 |
| 887 | ZNF611 |
| 888 | ZNF614 |
| 889 | ZNF623 |
| 890 | ZNF629 |
| 891 | ZNF638 |
| 892 | ZNF643 |
| 893 | ZNF646 |
| 894 | ZNF652 |
| 895 | ZNF654 |
| 896 | ZNF659 |
| 897 | ZNF665 |
| 898 | ZNF667 |
| 899 | ZNF668 |
| 900 | ZNF669 |
| 901 | ZNF671 |
| 902 | ZNF672 |
| 903 | ZNF673 |
| 904 | ZNF675 |
| 905 | ZNF682 |
| 906 | ZNF688 |
| 907 | ZNF692 |
| 908 | ZNF695 |
| 909 | ZNF696 |
| 910 | ZNF7 |
| 911 | ZNF701 |
| 912 | ZNF702 |
| 913 | ZNF706 |
| 914 | ZNF710 |
| 915 | ZNF711 |
| 916 | ZNF74 |
| 917 | ZNF75 |
| 918 | ZNF79 |
| 919 | ZNF8 |
| 920 | ZNF81 |
| 921 | ZNF83 |
| 922 | ZNF84 |
| 923 | ZNF85 |
| 924 | ZNF91 |
| 925 | ZNF93 |
| 926 | ZNF96 |
| 927 | ZNFN1A1 |
| 928 | ZSCAN5 |

TABLE 5
Ranked list of the TFs most frequently connected to the MGES
predicted by ARACNe and the TFs with consensus enrichment in
MGES promoters. TFs marked in blue are MRA-inferred TFs
with significant enrichment of binding site in MGES promoters,
and TFs marked in pink are enriched in DNA binding and
highly connected to MGES in the ARACNe inferred networks.
(a) MRA (b) DNA-Binding

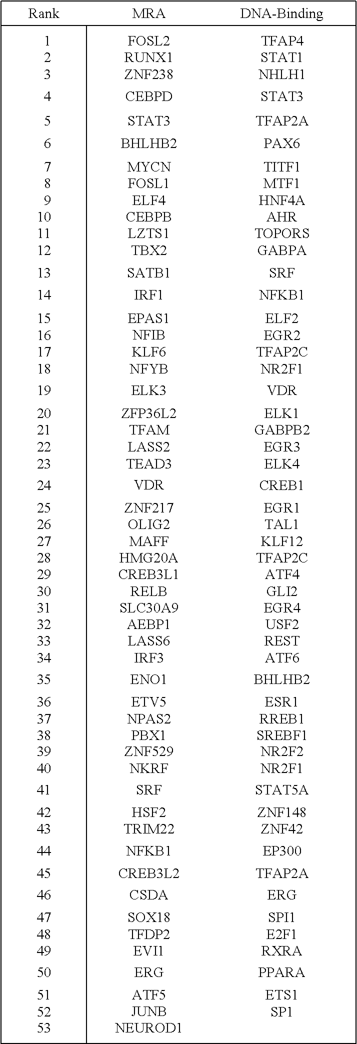

TABLE 6
Table 6. Regulon overlap analysis. The proportion of target genes
shared by pairs of TFs is significantly higher than expected by chance.
The top-right portion of the table shows the odds ratio and the
bottom-left portion the FET p-value for the contingency table of
the number of target genes specific and shared by each TF among
all genes tested by ARACNe as potential targets.

| | Stat3 | C/EBPβ | FosL2 | bHLH-B2 | Runx1 |
|---|---|---|---|---|---|
| Stat3 | | 4.81 | 10.6 | 9.39 | 6.29 |
| C/EBPβ | 1.77E−09 | | 13.9 | 6.63 | 13.5 |
| FosL2 | 3.00E−46 | 4.12E−40 | | 13.6 | 12.3 |
| bHLH-B2 | 2.15E−25 | 4.76E−17 | 1.76E−41 | | 5.87 |
| Runx1 | 9.56E−28 | 1.78E−44 | 2.26E−68 | 8.45E−17 | |

TABLE 7
Master Regulators inferred by the MRA and SLR
algorithms using the MGES signature..

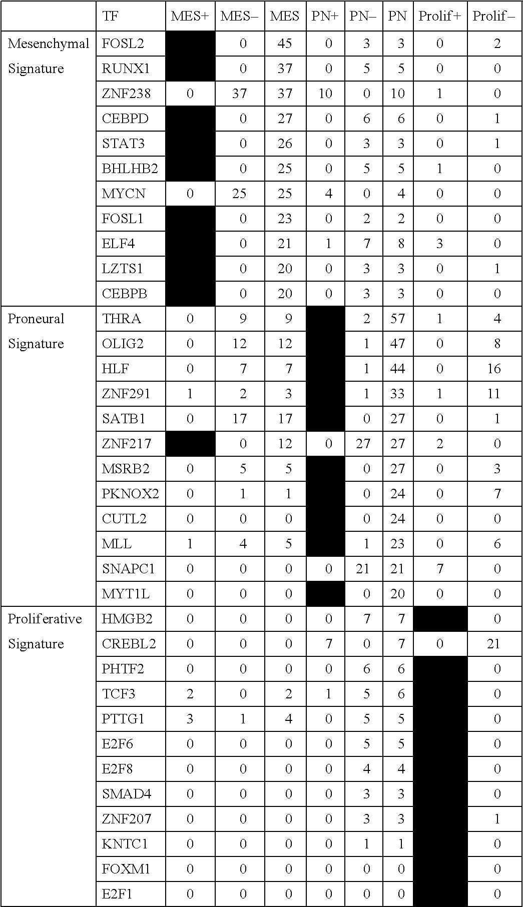

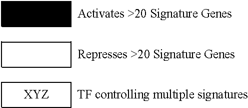

TABLE 8
Table 8. TFs with more than 20 connections with MGES, PNGES
and PROGES in the transcriptional networks. TFs marked in red
control more than one signature.

| | MGES Analysis | | | | |
|---|---|---|---|---|---|
| TF | MRA-rank | Overlap | p-value | SLR-rank | LR-Coeff |
| FOSL2 | 1 | 45 | 9.4E−39 | 5 | 0.21 |
| ZNF238 | 2 | 37 | 9.6E−28 | 2 | −0.34 |
| RUNX1 | 3 | 37 | 2.3E−24 | 4 | 0.13 |
| C/EBP(*) | 4 | 30 | 3.2E−19 | 1 | 0.40 |
| C/EBPδ | 5 | 27 | 1.2E−19 | 6 | 0.42 |
| STAT3 | 6 | 26 | 1.2E−16 | 7 | 0.40 |
| BHLHB2 | 7 | 25 | 7.8E−21 | 9 | 0.41 |
| MYCN | 8 | 25 | 6.2E−20 | 37 | −0.11 |
| FOSL1 | 9 | 23 | 3.6E−25 | 47 | 0.24 |
| ELF4 | 10 | 21 | 7.0E−09 | 34 | 0.1 |
| C/EBPβ | 11 | 20 | 2.2E−15 | 28 | 0.35 |
| LZTS1 | 12 | 20 | 3.8E−14 | 3 | 0.22 |
| TBX2 | 13 | 17 | 4.6E−12 | 23 | 0.17 |
| SATB1 | 14 | 17 | 1.4E−07 | 21 | −0.32 |
| IRF1 | 15 | 16 | 2.0E−11 | 19 | 0.48 |
| EPAS1 | 16 | 16 | 2.6E−09 | 16 | 0.21 |
| NFIB | 17 | 15 | 5.4E−07 | 8 | −0.32 |
| KLF6 | 18 | 14 | 2.0E−11 | — | 0.16 |
| NFYB | 19 | 14 | 3.5E−07 | 14 | −0.55 |
| ELK3 | 20 | 14 | 1.8E−06 | 53 | 0.24 |

‘—’ indicate that TF is not significant in regulon enrichment analysis and not included in SLR analysis
(*)The C/EBP metagene includes targets of both C/EBPβ and C/EBPδ

TABLE 9
shRNA mediated knock-down of MR-TFs in human glioma cells. a,
Enrichment of each MR-TF regulon on each TF-knock-down gene expression profile by
GSEA. Five additional TFs showing similar regulon size were added to the analysis as
negative controls: ATF2 for Stat3, SOX15 for C/EBPβ, ZNF500 for FosL2 and Runx1, and
ZNF277 for bHLH-B2. b, Enrichment of the MGES on genes downregulated after each MR-TF
knock-down. Shown is the normalized enrichment score (nES) and p-value estimated by permuting genes.
Table 9a

| | Silencing | | C/EBPβ | | Stat3 | | FosL2 | | bHLH-B2 | | Runx1 | |
|---|---|---|---|---|---|---|---|---|---|---|---|---|
| | Regulon | Size | nES | p-value | nES | p-value | nES | p-value | nES | p-value | nES | p-value |
| Module TFs | Stat3 | 366 | 2.49 | 0.0077 | 1.78 | 0.0397 | 3.29 | 0.0011 | 3.04 | 0.0016 | 2.18 | 0.0146 |
| | C/EBPβ | 209 | 1.91 | 0.0306 | 2.43 | 0.0092 | 2.30 | 0.0121 | 1.66 | 0.0539 | 3.62 | 0.0001 |
| | FosL2 | 403 | 3.83 | 0.0001 | 4.98 | <1E−4 | 3.83 | 0.0001 | 3.39 | 0.0007 | 3.66 | 0.0001 |
| | bHLH-B2 | 226 | 1.74 | 0.0429 | 0.59 | 0.2773 | 2.17 | 0.0171 | 1.39 | 0.0870 | 3.09 | 0.0014 |
| | Runx1 | 490 | 0.55 | 0.2910 | 2.41 | 0.0097 | 1.18 | 0.1267 | 1.98 | 0.0274 | 2.13 | 0.0168 |
| Control TFs | ATF2 | 386 | 1.54 | 0.0615 | −0.24 | 0.5965 | 1.42 | 0.0865 | 0.20 | 0.4134 | −1.49 | 0.9293 |
| | SOX15 | 213 | 0.28 | 0.3908 | −2.81 | 0.9976 | −0.12 | 0.5496 | −0.28 | 0.6070 | 0.70 | 0.2397 |
| | ZNF500 | 469 | −0.24 | 0.5970 | 0.20 | 0.4185 | 0.85 | 0.2012 | −0.74 | 0.7698 | 0.11 | 0.4543 |
| | ZNF277 | 238 | −0.79 | 0.7852 | −0.55 | 0.7116 | 0.41 | 0.3433 | 0.90 | 0.1849 | −0.91 | 0.8162 |

Table 9b

| | C/EBPβ | | Stat3 | | FosL2 | | bHLH-B2 | | Runx1 | |
|---|---|---|---|---|---|---|---|---|---|---|
| Silencing | nES | p value | nES | p value | nES | p value | nES | p value | nES | p value |
| MGES Enrich. | 3.23 | 0.0001 | 3.59 | 0.0001 | 3.92 | <1E−4 | 4.36 | 4.67E−06 | 3.82 | <1E−4 |

TABLE 10
Table 10. mRNA levels for C/EBPβ and Stat3 after silencing and over-
expression experiments. Shown is the median ± MAD and U-test
p-value for the C/EBPβ and Stat3 mRNA levels relative to non-target
shRNA transduced cells and mRNA levels for the
GAPDH mRNA housekeeping gene.

| | | C/EBPβ mRNA | | Stat3 mRNA | |
|---|---|---|---|---|---|
| | | Median ± | | Median ± | |
| | | MAD | p-value | MAD | p-value |
| Si- | C/EBPβ | 0.26 ± 0.119 | 0.00108 | 1.13 ± 0.43 | 0.153 |
| lencing | Stat3 | 0.87 ± 0.111 | 0.149 | 0.205 ± 0.052 | 0.00109 |
| | C/EBPβ × | 0.25 ± 0.163 | 0.00165 | 0.2 ± 0.074 | 0.00165 |
| | Stat3 | | | | |
| Over- | C/EBPβ | 45.53 ± 23.929 | 0.00781 | 0.89 ± 0.393 | 0.383 |
| ex- | Stat3 | 1.17 ± 0.541 | 0.313 | 3.79 ± 2.758 | 0.00781 |
| pression | CEBPβ × | 155.1 ± 57.11 | 0.00391 | 2.79 ± 1.171 | 0.00391 |
| | Stat3 | | | | |

TABLE 11
Table 11. GSEA of ARACNe regulons on the gene expression profile rank-
sorted by its correlation with the mRNA levels of C/EBPβ, Stat3, and C/EBPβ × Stat3
(the metagene). Shown is the regulon size, normalized enrichment score (nES), sample
permutation-based p-value and leading-edge odds ratio (LEOR) for the MR-TFs:
C/EBPβ, Stat3, FosL2, bHLH-B2 and Runx1; and 5 randomly selected control TFs with
comparable number of target genes.

| | C/EBPβ mRNA | | | Stat3 mRNA | | | C/EBPβ × Stat3 | | |
|---|---|---|---|---|---|---|---|---|---|
| | nES | p-value | LEOR | nES | p-value | LEOR | nES | p-value | LEOR |
| C/EBPβ | 2.05 | 0.0290 | 2.29 | 3.17 | 0.0008 | 3.46 | 2.67 | 0.0038 | 2.75 |
| Stat3 | 1.91 | 0.0340 | 1.94 | 3.21 | 0.0007 | 2.38 | 2.60 | 0.0046 | 2.56 |
| FosL2 | 2.03 | 0.0210 | 2.35 | 3.60 | 0.0002 | 3.51 | 3.02 | 0.0013 | 3.26 |
| bHLH- | 2.07 | 0.0190 | 2.37 | 3.48 | 0.0002 | 3.28 | 2.82 | 0.0024 | 2.91 |
| B2 | | | | | | | | | |
| Runx1 | 2.16 | 0.0170 | 1.81 | 4.04 | <1E−4 | 2.56 | 3.24 | 0.0006 | 2.25 |
| ATF2 | −1.37 | 0.8800 | — | −1.43 | 0.9220 | — | −1.57 | 0.9290 | — |
| SOX15 | 0.15 | 0.4370 | — | 0.36 | 0.3850 | — | 0.42 | 0.3460 | — |
| ZNF500 | −1.50 | 0.9190 | — | −0.77 | 0.7530 | — | −1.34 | 0.8990 | — |
| ZNF277 | −0.29 | 0.6060 | — | 0.56 | 0.3120 | — | 0.20 | 0.4250 | — |

TABLE 12
List of 884 genes in TCGA Worst Prognosis Signature (TWPS),
identified by differential expression analysis
(p < 0.05 based on Student's t-test) between 77
low- and 21 high-survival samples in the TCGA dataset.

| Rank | Gene ID | p-value | Overlap |
|---|---|---|---|
| 1 | IL8 | 1.1E−06 | |
| 2 | PTX3 | 2.8E−06 | |
| 3 | EFEMP2 | 5.9E−06 | 1 |
| 4 | SSR3 | 6.7E−06 | |
| 5 | TAGLN2 | 6.8E−06 | |
| 6 | PDPN | 1.7E−05 | 1 |
| 7 | EMP3 | 2.7E−05 | 1 |
| 8 | TFRC | 2.9E−05 | |
| 9 | GLT8D1 | 5.2E−05 | |
| 10 | PSMD13 | 5.3E−05 | |
| 11 | ADM | 5.8E−05 | |
| 12 | LGALS8 | 6.6E−05 | |
| 13 | PLOD2 | 7.3E−05 | |
| 14 | CHI3L1 | 7.4E−05 | 1 |
| 15 | TMEM22 | 8.0E−05 | |
| 16 | NRN1 | 8.5E−05 | |
| 17 | LGALS1 | 8.7E−05 | |
| 18 | RIG | 9.0E−05 | |
| 19 | IGFBP2 | 9.6E−05 | |
| 20 | C6orf62 | 1.0E−04 | |
| 21 | MT1M | 1.2E−04 | |
| 22 | LDHA | 1.4E−04 | |
| 23 | NOL3 | 1.5E−04 | |
| 24 | TIMP1 | 1.6E−04 | 1 |
| 25 | SCG2 | 1.7E−04 | |
| 26 | CLIC1 | 1.7E−04 | |
| 27 | ARFIP2 | 1.7E−04 | |
| 28 | HFE | 2.0E−04 | |
| 29 | COPB1 | 2.2E−04 | |
| 30 | MDK | 2.5E−04 | |
| 31 | DUSP6 | 2.5E−04 | |
| 32 | NSUN5C | 2.7E−04 | |
| 33 | KRT10 | 2.9E−04 | |
| 34 | PGK1 | 3.0E−04 | |
| 35 | DKK3 | 3.2E−04 | |
| 36 | POLR1D | 3.2E−04 | |
| 37 | FAS | 3.4E−04 | |
| 38 | PCNP | 3.6E−04 | |
| 39 | NSUN5 | 4.2E−04 | |
| 40 | DYNLT3 | 4.2E−04 | |
| 41 | TUBB2A | 4.4E−04 | |
| 42 | UPP1 | 4.4E−04 | |
| 43 | ABHD3 | 4.5E−04 | |
| 44 | SPP1 | 5.0E−04 | |
| 45 | DDIT3 | 5.1E−04 | |
| 46 | NNMT | 5.2E−04 | |
| 47 | SPA17 | 5.5E−04 | |
| 48 | SSBP2 | 5.5E−04 | |
| 49 | DLAT | 5.6E−04 | |
| 50 | DRG2 | 5.6E−04 | |
| 51 | FAM3C | 5.8E−04 | |
| 52 | ATP2B1 | 6.4E−04 | |
| 53 | DNAJB9 | 6.4E−04 | |
| 54 | ARNTL | 6.4E−04 | |
| 55 | CD63 | 6.4E−04 | |
| 56 | MT1F | 6.6E−04 | |
| 57 | FLJ11286 | 6.8E−04 | |
| 58 | SDC2 | 6.8E−04 | |
| 59 | RAB33B | 7.5E−04 | |
| 60 | PIGB | 7.7E−04 | |
| 61 | DERA | 7.9E−04 | |
| 62 | PEX7 | 8.1E−04 | |
| 63 | RIOK3 | 8.2E−04 | |
| 64 | KIAA0409 | 8.4E−04 | |
| 65 | HRASLS3 | 9.5E−04 | |
| 66 | TAF9 | 9.5E−04 | |
| 67 | FZD7 | 9.5E−04 | |
| 68 | SLC25A24 | 9.6E−04 | |
| 69 | TRIP4 | 9.6E−04 | |
| 70 | FRAG1 | 9.6E−04 | |
| 71 | CARS | 9.7E−04 | |
| 72 | EGLN3 | 9.7E−04 | |
| 73 | FAHD2A | 9.9E−04 | |
| 74 | ANGPT1 | 1.0E−03 | |
| 75 | FLJ11506 | 1.0E−03 | |
| 76 | CD44 | 1.0E−03 | |
| 77 | GBE1 | 1.0E−03 | |
| 78 | NTAN1 | 1.0E−03 | |
| 79 | SLC35A3 | 1.0E−03 | |
| 80 | LOC390940 | 1.1E−03 | |
| 81 | REXO2 | 1.1E−03 | |
| 82 | FLNC | 1.1E−03 | |
| 83 | RPL23AP7 | 1.1E−03 | |
| 84 | FABP3 | 1.1E−03 | |
| 85 | AACS | 1.2E−03 | |
| 86 | SLC38A6 | 1.2E−03 | |
| 87 | PTS | 1.2E−03 | |
| 88 | SLC43A3 | 1.2E−03 | |
| 89 | HRH4 | 1.2E−03 | |
| 90 | TRIB3 | 1.2E−03 | |
| 91 | AP3S1 | 1.2E−03 | |
| 92 | C13orf18 | 1.2E−03 | |
| 93 | COQ10B | 1.2E−03 | |
| 94 | RGN | 1.2E−03 | |
| 95 | GTF2H5 | 1.3E−03 | |
| 96 | NUP160 | 1.4E−03 | |
| 97 | DDX47 | 1.4E−03 | |
| 98 | LSM5 | 1.5E−03 | |
| 99 | TPI1 | 1.5E−03 | |
| 100 | KIAA0495 | 1.5E−03 | |
| 101 | S100A13 | 1.6E−03 | |
| 102 | ARTS-1 | 1.6E−03 | |
| 103 | CYCS | 1.6E−03 | |
| 104 | TMEM158 | 1.6E−03 | |
| 105 | IL1RAPL2 | 1.7E−03 | |
| 106 | HEMK1 | 1.7E−03 | |
| 107 | C3orf60 | 1.7E−03 | |
| 108 | NUP98 | 1.8E−03 | |
| 109 | TMBIM1 | 1.8E−03 | |
| 110 | HIGD1A | 1.8E−03 | |
| 111 | SSH3 | 1.9E−03 | |
| 112 | MDS032 | 1.9E−03 | |
| 113 | EIF1 | 1.9E−03 | |
| 114 | DALRD3 | 1.9E−03 | |
| 115 | SYPL1 | 2.0E−03 | |
| 116 | APOE | 2.0E−03 | |
| 117 | PTPN12 | 2.0E−03 | |
| 118 | TOM1L1 | 2.0E−03 | |
| 119 | EIF4E2 | 2.0E−03 | |
| 120 | C7orf25 | 2.0E−03 | |
| 121 | KIAA0895 | 2.0E−03 | |
| 122 | HEBP1 | 2.1E−03 | |
| 123 | ECHDC2 | 2.1E−03 | |
| 124 | IQCG | 2.2E−03 | |
| 125 | FKBP9 | 2.2E−03 | |
| 126 | SOD2 | 2.3E−03 | |
| 127 | RBP1 | 2.3E−03 | |
| 128 | MRPL17 | 2.3E−03 | |
| 129 | SLC2A3 | 2.3E−03 | |
| 130 | DUS4L | 2.5E−03 | |
| 131 | CCDC109B | 2.5E−03 | |
| 132 | C12orf29 | 2.6E−03 | |
| 133 | FBXO17 | 2.6E−03 | |
| 134 | CAMK2N1 | 2.6E−03 | |
| 135 | RIC8A | 2.6E−03 | |
| 136 | HK2 | 2.6E−03 | |
| 137 | PLSCR1 | 2.7E−03 | |
| 138 | G0S2 | 2.7E−03 | |
| 139 | DCTD | 2.8E−03 | |
| 140 | SDHD | 2.8E−03 | |
| 141 | MT1E | 2.8E−03 | |
| 142 | POLR2L | 2.8E−03 | |
| 143 | OSTM1 | 2.8E−03 | |
| 144 | F3 | 2.9E−03 | |
| 145 | RNH1 | 2.9E−03 | |
| 146 | CCL20 | 2.9E−03 | |
| 147 | CSRP1 | 2.9E−03 | |
| 148 | FLJ22222 | 2.9E−03 | |
| 149 | PDLIM3 | 2.9E−03 | |
| 150 | ATG12 | 3.0E−03 | |
| 151 | COG5 | 3.0E−03 | |
| 152 | CBR1 | 3.1E−03 | |
| 153 | MTRR | 3.2E−03 | |
| 154 | MAFF | 3.3E−03 | |
| 155 | LIN7C | 3.3E−03 | |
| 156 | SPRY2 | 3.4E−03 | |
| 157 | BCL2A1 | 3.4E−03 | |
| 158 | BCAP29 | 3.4E−03 | |
| 159 | STEAP3 | 3.4E−03 | 1 |
| 160 | CTNNB1 | 3.4E−03 | |
| 161 | CYP3A43 | 3.5E−03 | |
| 162 | SMS | 3.6E−03 | |
| 163 | GRPEL1 | 3.6E−03 | |
| 164 | DOK3 | 3.6E−03 | |
| 165 | CCL2 | 3.6E−03 | |
| 166 | ARSJ | 3.6E−03 | |
| 167 | ITGA7 | 3.6E−03 | 1 |
| 168 | FKBP2 | 3.7E−03 | |
| 169 | WWTR1 | 3.7E−03 | |
| 170 | PGCP | 3.7E−03 | |
| 171 | VLDLR | 3.7E−03 | |
| 172 | STK19 | 3.8E−03 | |
| 173 | LOC201229 | 3.8E−03 | |
| 174 | TFPI | 3.8E−03 | |
| 175 | POP5 | 3.8E−03 | |
| 176 | GAP43 | 3.8E−03 | |
| 177 | FAM62A | 3.8E−03 | |
| 178 | MT1G | 3.8E−03 | |
| 179 | TUSC2 | 3.9E−03 | |
| 180 | MET | 3.9E−03 | |
| 181 | EPS8 | 3.9E−03 | |
| 182 | C19orf10 | 4.0E−03 | |
| 183 | ATP13A3 | 4.1E−03 | |
| 184 | UNC84A | 4.1E−03 | |
| 185 | GRB10 | 4.1E−03 | |
| 186 | STK17A | 4.1E−03 | |
| 187 | RQCD1 | 4.2E−03 | |
| 188 | C19orf53 | 4.3E−03 | |
| 189 | EXOC3 | 4.3E−03 | |
| 190 | HSD17B12 | 4.3E−03 | |
| 191 | PDGFA | 4.3E−03 | 1 |
| 192 | RPL14 | 4.4E−03 | |
| 193 | HES1 | 4.4E−03 | |
| 194 | TMEM41B | 4.4E−03 | |
| 195 | SYNJ2 | 4.5E−03 | |
| 196 | TRAM1 | 4.5E−03 | |
| 197 | RCP9 | 4.5E−03 | |
| 198 | SP100 | 4.6E−03 | |
| 199 | TNFRSF12A | 4.6E−03 | |
| 200 | VAMP4 | 4.6E−03 | |
| 201 | CDC5L | 4.7E−03 | |
| 202 | CHL1 | 4.7E−03 | |
| 203 | ANGPTL4 | 4.8E−03 | 1 |
| 204 | TNPO1 | 4.8E−03 | |
| 205 | TCEB1 | 4.8E−03 | |
| 206 | HBXIP | 4.9E−03 | |
| 207 | DNPEP | 4.9E−03 | |
| 208 | ACOX2 | 4.9E−03 | |
| 209 | TNFAIP6 | 4.9E−03 | |
| 210 | ARL4C | 5.0E−03 | |
| 211 | FAM18B | 5.0E−03 | |
| 212 | LITAF | 5.1E−03 | |
| 213 | PMP22 | 5.2E−03 | |
| 214 | ADFP | 5.2E−03 | |
| 215 | RRAS2 | 5.2E−03 | |
| 216 | TSPAN13 | 5.2E−03 | |
| 217 | TIPARP | 5.3E−03 | |
| 218 | ARPC3 | 5.3E−03 | |
| 219 | NUP37 | 5.3E−03 | |
| 220 | TBCA | 5.3E−03 | |
| 221 | S100A4 | 5.3E−03 | |
| 222 | NSUN5B | 5.3E−03 | |
| 223 | GOLT1B | 5.3E−03 | |
| 224 | UGCG | 5.3E−03 | |
| 225 | HMBS | 5.3E−03 | |
| 226 | ISG20 | 5.3E−03 | |
| 227 | IFT57 | 5.3E−03 | |
| 228 | CALR | 5.5E−03 | |
| 229 | TBCE | 5.5E−03 | |
| 230 | MEOX2 | 5.5E−03 | |
| 231 | CSRP2 | 5.5E−03 | |
| 232 | PDIA4 | 5.6E−03 | |
| 233 | SMEK2 | 5.6E−03 | |
| 234 | OBSL1 | 5.7E−03 | |
| 235 | CD164 | 5.7E−03 | |
| 236 | PRPS2 | 5.7E−03 | |
| 237 | PTDSS2 | 5.8E−03 | |
| 238 | SPAG4 | 5.8E−03 | |
| 239 | RBPMS | 5.8E−03 | |
| 240 | FN3KRP | 5.9E−03 | |
| 241 | MXRA7 | 5.9E−03 | |
| 242 | HEXB | 6.0E−03 | |
| 243 | MGC14376 | 6.0E−03 | |
| 244 | ATP5L | 6.0E−03 | |
| 245 | TMEM38B | 6.0E−03 | |
| 246 | GRB14 | 6.1E−03 | |
| 247 | BUD31 | 6.1E−03 | |
| 248 | NDP | 6.1E−03 | |
| 249 | GCA | 6.1E−03 | |
| 250 | CLN5 | 6.2E−03 | |
| 251 | ASB4 | 6.2E−03 | |
| 252 | TSPAN4 | 6.2E−03 | |
| 253 | S100A6 | 6.2E−03 | |
| 254 | ILK | 6.2E−03 | |
| 255 | GNG12 | 6.2E−03 | |
| 256 | BRP44L | 6.4E−03 | |
| 257 | ABCB9 | 6.4E−03 | |
| 258 | MRPL49 | 6.4E−03 | |
| 259 | RNF14 | 6.4E−03 | |
| 260 | ARL8B | 6.4E−03 | |
| 261 | TBL2 | 6.4E−03 | |
| 262 | NXPH4 | 6.5E−03 | |
| 263 | CYP3A7 | 6.5E−03 | |
| 264 | CHCHD2 | 6.6E−03 | |
| 265 | LECT1 | 6.7E−03 | |
| 266 | SLC2A1 | 6.7E−03 | |
| 267 | COPS2 | 6.7E−03 | |
| 268 | ARF6 | 6.7E−03 | |
| 269 | MAOB | 6.7E−03 | |
| 270 | SMYD2 | 6.8E−03 | |
| 271 | SLC2A10 | 6.8E−03 | |
| 272 | CD58 | 6.8E−03 | |
| 273 | C19orf42 | 6.8E−03 | |
| 274 | IL1RAP | 6.9E−03 | |
| 275 | MPV17 | 6.9E−03 | |
| 276 | NPY2R | 6.9E−03 | |
| 277 | TIMM10 | 7.0E−03 | |
| 278 | PIPOX | 7.1E−03 | |
| 279 | PUS7 | 7.3E−03 | |
| 280 | ORMDL2 | 7.3E−03 | |
| 281 | HOXC6 | 7.3E−03 | |
| 282 | MAB21L2 | 7.3E−03 | |
| 283 | TM2D1 | 7.3E−03 | |
| 284 | GNAT3 | 7.3E−03 | |
| 285 | HOMER1 | 7.4E−03 | |
| 286 | C5orf21 | 7.5E−03 | |
| 287 | AP1S1 | 7.5E−03 | |
| 288 | TCTA | 7.6E−03 | |
| 289 | TRIM5 | 7.6E−03 | |
| 290 | UQCRQ | 7.6E−03 | |
| 291 | ACTL6A | 7.7E−03 | |
| 292 | MYD88 | 7.7E−03 | |
| 293 | FXC1 | 7.8E−03 | |
| 294 | FLOT1 | 7.8E−03 | |
| 295 | CA12 | 7.8E−03 | 1 |
| 296 | HUS1 | 8.0E−03 | |
| 297 | EN2 | 8.0E−03 | |
| 298 | ITPR1 | 8.0E−03 | |
| 299 | HOXA1 | 8.2E−03 | |
| 300 | WEE1 | 8.3E−03 | |
| 301 | CUL5 | 8.3E−03 | |
| 302 | LRRC16 | 8.3E−03 | |
| 303 | CAST | 8.3E−03 | |
| 304 | S100A10 | 8.4E−03 | |
| 305 | FXYD3 | 8.4E−03 | |
| 306 | UEVLD | 8.4E−03 | |
| 307 | PRNP | 8.5E−03 | |
| 308 | TAPBPL | 8.5E−03 | |
| 309 | PI3 | 8.5E−03 | 1 |
| 310 | IL1A | 8.6E−03 | |
| 311 | SUB1 | 8.6E−03 | |
| 312 | PTRH2 | 8.6E−03 | |
| 313 | TXN | 8.6E−03 | |
| 314 | MPL | 8.7E−03 | |
| 315 | GSTO1 | 8.8E−03 | |
| 316 | KRAS | 8.8E−03 | |
| 317 | CNDP2 | 8.8E−03 | |
| 318 | IGFBP5 | 8.9E−03 | |
| 319 | MYCBP | 8.9E−03 | |
| 320 | ANXA2 | 9.0E−03 | |
| 321 | TANK | 9.1E−03 | |
| 322 | ZNF226 | 9.1E−03 | |
| 323 | CAPG | 9.1E−03 | |
| 324 | TOB1 | 9.1E−03 | |
| 325 | C3orf28 | 9.2E−03 | |
| 326 | PKM2 | 9.2E−03 | |
| 327 | GAPDH | 9.2E−03 | |
| 328 | POLR2A | 9.3E−03 | |
| 329 | SNUPN | 9.4E−03 | |
| 330 | CHPF | 9.4E−03 | |
| 331 | EIF5 | 9.4E−03 | |
| 332 | CD151 | 9.4E−03 | 1 |
| 333 | AK2 | 9.5E−03 | |
| 334 | LYPLA1 | 9.6E−03 | |
| 335 | CNR2 | 9.6E−03 | |
| 336 | CRTAP | 9.7E−03 | |
| 337 | ATF3 | 9.7E−03 | |
| 338 | RPL37A | 9.8E−03 | |
| 339 | ICT1 | 9.8E−03 | |
| 340 | PDCD10 | 9.8E−03 | |
| 341 | TNFRSF11B | 9.8E−03 | |
| 342 | MOSC2 | 9.8E−03 | |
| 343 | CXCL3 | 9.8E−03 | |
| 344 | TAF10 | 9.8E−03 | |
| 345 | IKBKE | 9.9E−03 | |
| 346 | C12orf41 | 9.9E−03 | |
| 347 | FLJ10292 | 9.9E−03 | |
| 348 | PRR13 | 9.9E−03 | |
| 349 | SLFN12 | 1.0E−02 | |
| 350 | NPAS3 | 1.0E−02 | |
| 351 | SCARB1 | 1.0E−02 | |
| 352 | ACACA | 1.0E−02 | |
| 353 | SPCS1 | 1.0E−02 | |
| 354 | IPO7 | 1.0E−02 | |
| 355 | CA3 | 1.0E−02 | |
| 356 | GGCX | 1.0E−02 | |
| 357 | PSMA1 | 1.0E−02 | |
| 358 | ANXA5 | 1.1E−02 | |
| 359 | SLC30A5 | 1.1E−02 | |
| 360 | ANGPT2 | 1.1E−02 | 1 |
| 361 | AP4S1 | 1.1E−02 | |
| 362 | PLA2G2A | 1.1E−02 | |
| 363 | MPP6 | 1.1E−02 | |
| 364 | CCL8 | 1.1E−02 | |
| 365 | CTTN | 1.1E−02 | |
| 366 | SERPINB6 | 1.1E−02 | |
| 367 | CDR2 | 1.1E−02 | |
| 368 | LEPR | 1.1E−02 | |
| 369 | TMBIM4 | 1.1E−02 | |
| 370 | SSX2IP | 1.1E−02 | |
| 371 | RYR3 | 1.1E−02 | 1 |
| 372 | TPST1 | 1.1E−02 | |
| 373 | SNRPA1 | 1.2E−02 | |
| 374 | TMEM5 | 1.2E−02 | |
| 375 | ALG8 | 1.2E−02 | |
| 376 | TIMM8B | 1.2E−02 | |
| 377 | PARVA | 1.2E−02 | |
| 378 | NDFIP1 | 1.2E−02 | |
| 379 | THOC7 | 1.2E−02 | |
| 380 | TBC1D15 | 1.2E−02 | |
| 381 | DNAJC6 | 1.2E−02 | |
| 382 | EPPB9 | 1.2E−02 | |
| 383 | LSM4 | 1.2E−02 | |
| 384 | GLRA1 | 1.2E−02 | |
| 385 | UBB | 1.2E−02 | |
| 386 | MINA | 1.2E−02 | |
| 387 | TRAPPC4 | 1.2E−02 | |
| 388 | SAR1B | 1.3E−02 | |
| 389 | ANGEL2 | 1.3E−02 | |
| 390 | TAF1B | 1.3E−02 | |
| 391 | DIRAS3 | 1.3E−02 | |
| 392 | MLX | 1.3E−02 | |
| 393 | HSPB7 | 1.3E−02 | |
| 394 | C17orf75 | 1.3E−02 | |
| 395 | C5orf28 | 1.3E−02 | |
| 396 | CEBPB | 1.3E−02 | |
| 397 | TRSPAP1 | 1.3E−02 | |
| 398 | RFK | 1.3E−02 | |
| 399 | CNIH | 1.3E−02 | |
| 400 | HSPA5 | 1.3E−02 | |
| 401 | GNS | 1.3E−02 | |
| 402 | CHPT1 | 1.3E−02 | |
| 403 | ELOVL6 | 1.3E−02 | |
| 404 | BNIP3 | 1.3E−02 | |
| 405 | COX5B | 1.3E−02 | |
| 406 | G6PC3 | 1.3E−02 | |
| 407 | ZNF143 | 1.4E−02 | |
| 408 | DUSP3 | 1.4E−02 | |
| 409 | YIPF2 | 1.4E−02 | |
| 410 | DOHH | 1.4E−02 | |
| 411 | GNAT1 | 1.4E−02 | |
| 412 | ARF5 | 1.4E−02 | |
| 413 | PSPH | 1.4E−02 | |
| 414 | OSMR | 1.4E−02 | 1 |
| 415 | GALNT7 | 1.4E−02 | |
| 416 | HSPE1 | 1.4E−02 | |
| 417 | SLC39A14 | 1.4E−02 | |
| 418 | FTL | 1.4E−02 | |
| 419 | ANXA2P2 | 1.4E−02 | |
| 420 | SMC4 | 1.4E−02 | |
| 421 | PDK1 | 1.4E−02 | |
| 422 | PSMC6 | 1.4E−02 | |
| 423 | TPD52L1 | 1.4E−02 | |
| 424 | PCDH8 | 1.4E−02 | |
| 425 | ACTN1 | 1.5E−02 | 1 |
| 426 | SWAP70 | 1.5E−02 | |
| 427 | FER1L4 | 1.5E−02 | |
| 428 | CHRNA2 | 1.5E−02 | |
| 429 | C17orf42 | 1.5E−02 | |
| 430 | MAS1 | 1.5E−02 | |
| 431 | IRF7 | 1.5E−02 | |
| 432 | PDCD6 | 1.5E−02 | |
| 433 | DHRS7B | 1.5E−02 | |
| 434 | TMEM9B | 1.5E−02 | |
| 435 | GLRX | 1.5E−02 | |
| 436 | TMED7 | 1.5E−02 | |
| 437 | CCDC59 | 1.5E−02 | |
| 438 | CAPZA2 | 1.5E−02 | |
| 439 | ZNF552 | 1.5E−02 | |
| 440 | BHLHB2 | 1.5E−02 | 1 |
| 441 | FAM96B | 1.5E−02 | |
| 442 | GPNMB | 1.5E−02 | |
| 443 | SMPD1 | 1.5E−02 | |
| 444 | TMCO3 | 1.5E−02 | |
| 445 | SNX3 | 1.5E−02 | |
| 446 | CHST2 | 1.5E−02 | |
| 447 | MGC3196 | 1.5E−02 | |
| 448 | POLR2G | 1.5E−02 | |
| 449 | LRP12 | 1.6E−02 | |
| 450 | CD47 | 1.6E−02 | |
| 451 | EXT2 | 1.6E−02 | |
| 452 | CHMP2A | 1.6E−02 | |
| 453 | EFEMP1 | 1.6E−02 | |
| 454 | TMEM14A | 1.6E−02 | |
| 455 | IGF2BP3 | 1.6E−02 | |
| 456 | BCL3 | 1.6E−02 | 1 |
| 457 | CHN2 | 1.6E−02 | |
| 458 | RARRES2 | 1.6E−02 | |
| 459 | FNTA | 1.7E−02 | |
| 460 | CPD | 1.7E−02 | |
| 461 | CLEC5A | 1.7E−02 | |
| 462 | LEF1 | 1.7E−02 | |
| 463 | SNX10 | 1.7E−02 | |
| 464 | PCDH9 | 1.7E−02 | |
| 465 | ABCC3 | 1.7E−02 | |
| 466 | ARHGAP29 | 1.7E−02 | |
| 467 | ELOVL2 | 1.7E−02 | |
| 468 | NENF | 1.7E−02 | |
| 469 | UNC50 | 1.7E−02 | |
| 470 | APITD1 | 1.7E−02 | |
| 471 | ARPC4 | 1.7E−02 | |
| 472 | VIL2 | 1.7E−02 | |
| 473 | USP33 | 1.7E−02 | |
| 474 | POLR2C | 1.7E−02 | |
| 475 | PAM | 1.7E−02 | |
| 476 | LZTFL1 | 1.7E−02 | |
| 477 | UTP6 | 1.7E−02 | |
| 478 | HIG2 | 1.7E−02 | |
| 479 | MIA2 | 1.7E−02 | |
| 480 | STK3 | 1.8E−02 | |
| 481 | CPEB1 | 1.8E−02 | |
| 482 | GADD45B | 1.8E−02 | |
| 483 | RGS3 | 1.8E−02 | |
| 484 | C14orf109 | 1.8E−02 | |
| 485 | CFLAR | 1.8E−02 | |
| 486 | SLC25A20 | 1.8E−02 | |
| 487 | VAMP5 | 1.8E−02 | |
| 488 | COMMD8 | 1.8E−02 | |
| 489 | ST8SIA5 | 1.8E−02 | |
| 490 | SLC33A1 | 1.8E−02 | |
| 491 | IFRD1 | 1.8E−02 | |
| 492 | PLP2 | 1.8E−02 | |
| 493 | PLS3 | 1.8E−02 | |
| 494 | PSMC3IP | 1.8E−02 | |
| 495 | POSTN | 1.8E−02 | |
| 496 | PCBD1 | 1.9E−02 | |
| 497 | CHI3L2 | 1.9E−02 | |
| 498 | DUSP14 | 1.9E−02 | |
| 499 | LYRM2 | 1.9E−02 | |
| 500 | PPIC | 1.9E−02 | |
| 501 | ATP5S | 1.9E−02 | |
| 502 | CFI | 1.9E−02 | |
| 503 | GMPR | 1.9E−02 | |
| 504 | ARMET | 1.9E−02 | |
| 505 | HSP90B1 | 1.9E−02 | |
| 506 | SLC4A3 | 1.9E−02 | |
| 507 | CASP3 | 1.9E−02 | |
| 508 | RHEB | 1.9E−02 | |
| 509 | ATPBD1C | 1.9E−02 | |
| 510 | MAP7 | 1.9E−02 | |
| 511 | MGC5618 | 1.9E−02 | |
| 512 | ARPC5 | 1.9E−02 | |
| 513 | ACAA2 | 1.9E−02 | |
| 514 | FKBP1B | 1.9E−02 | |
| 515 | Magmas | 2.0E−02 | |
| 516 | UBE2NL | 2.0E−02 | |
| 517 | MTCH2 | 2.0E−02 | |
| 518 | AZGP1 | 2.0E−02 | |
| 519 | PPP1R15A | 2.0E−02 | |
| 520 | BBS10 | 2.0E−02 | |
| 521 | HOXA5 | 2.0E−02 | |
| 522 | HS2ST1 | 2.0E−02 | |
| 523 | ATP6V1D | 2.0E−02 | |
| 524 | C11orf58 | 2.0E−02 | |
| 525 | STOML1 | 2.0E−02 | |
| 526 | HRH1 | 2.0E−02 | 1 |
| 527 | TGFBI | 2.0E−02 | |
| 528 | ATP5G1 | 2.0E−02 | |
| 529 | CASP4 | 2.1E−02 | |
| 530 | TIAM2 | 2.1E−02 | |
| 531 | RGS16 | 2.1E−02 | |
| 532 | SNAPC5 | 2.1E−02 | |
| 533 | GLS | 2.1E−02 | |
| 534 | PUS1 | 2.1E−02 | |
| 535 | CHMP2B | 2.1E−02 | |
| 536 | C9orf53 | 2.1E−02 | |
| 537 | RRAS | 2.1E−02 | 1 |
| 538 | CHCHD7 | 2.1E−02 | |
| 539 | AKAP12 | 2.1E−02 | |
| 540 | LARP6 | 2.1E−02 | |
| 541 | PPP3CC | 2.1E−02 | |
| 542 | ATP5F1 | 2.2E−02 | |
| 543 | CLDN10 | 2.2E−02 | |
| 544 | ALAS1 | 2.2E−02 | |
| 545 | CHN1 | 2.2E−02 | |
| 546 | SACM1L | 2.2E−02 | |
| 547 | IFI44 | 2.2E−02 | |
| 548 | PSMD14 | 2.2E−02 | |
| 549 | IL6 | 2.2E−02 | |
| 550 | FABP7 | 2.2E−02 | |
| 551 | ZNF593 | 2.2E−02 | |
| 552 | RS1 | 2.2E−02 | |
| 553 | EFHC2 | 2.2E−02 | |
| 554 | B3GALNT1 | 2.3E−02 | |
| 555 | GRPR | 2.3E−02 | |
| 556 | EI24 | 2.3E−02 | |
| 557 | GINS4 | 2.3E−02 | |
| 558 | DLG1 | 2.3E−02 | |
| 559 | LTBP1 | 2.3E−02 | |
| 560 | LOX | 2.3E−02 | |
| 561 | GLIPR1 | 2.3E−02 | |
| 562 | P4HA2 | 2.3E−02 | |
| 563 | RIMBP2 | 2.4E−02 | |
| 564 | MRPL2 | 2.4E−02 | |
| 565 | PLA2G5 | 2.4E−02 | 1 |
| 566 | IER3IP1 | 2.4E−02 | |
| 567 | MCFD2 | 2.4E−02 | |
| 568 | SRPX2 | 2.4E−02 | |
| 569 | EBNA1BP2 | 2.4E−02 | |
| 570 | RPL39L | 2.4E−02 | |
| 571 | TMED9 | 2.4E−02 | |
| 572 | RNASE1 | 2.4E−02 | |
| 573 | C14orf2 | 2.4E−02 | |
| 574 | BHLHB9 | 2.4E−02 | |
| 575 | ARL1 | 2.4E−02 | |
| 576 | TSC22D2 | 2.4E−02 | |
| 577 | EFNB2 | 2.4E−02 | 1 |
| 578 | PTPN21 | 2.4E−02 | |
| 579 | YAP1 | 2.5E−02 | |
| 580 | WSB2 | 2.5E−02 | |
| 581 | IL1RN | 2.5E−02 | |
| 582 | CYBRD1 | 2.5E−02 | |
| 583 | GUK1 | 2.5E−02 | |
| 584 | ORC5L | 2.5E−02 | |
| 585 | XPOT | 2.5E−02 | |
| 586 | LIAS | 2.5E−02 | |
| 587 | ITGB1BP1 | 2.5E−02 | |
| 588 | CTBS | 2.5E−02 | |
| 589 | GTF2H1 | 2.5E−02 | |
| 590 | TMEM106C | 2.5E−02 | |
| 591 | COX17 | 2.5E−02 | |
| 592 | HOMER3 | 2.5E−02 | 1 |
| 593 | SDC4 | 2.5E−02 | |
| 594 | DUSP5 | 2.5E−02 | |
| 595 | GPX3 | 2.6E−02 | |
| 596 | APIP | 2.6E−02 | |
| 597 | IFRD2 | 2.6E−02 | |
| 598 | RPA3 | 2.6E−02 | |
| 599 | GGH | 2.6E−02 | |
| 600 | HOXC10 | 2.6E−02 | |
| 601 | CD99 | 2.6E−02 | |
| 602 | HPCAL1 | 2.6E−02 | |
| 603 | FAH | 2.6E−02 | |
| 604 | PPFIA4 | 2.6E−02 | |
| 605 | C14orf45 | 2.6E−02 | |
| 606 | MC3R | 2.6E−02 | |
| 607 | PIGG | 2.6E−02 | |
| 608 | PCSK5 | 2.6E−02 | |
| 609 | ITGA5 | 2.6E−02 | 1 |
| 610 | RBKS | 2.7E−02 | |
| 611 | C18orf10 | 2.7E−02 | |
| 612 | AUH | 2.7E−02 | |
| 613 | CD97 | 2.7E−02 | 1 |
| 614 | RNF7 | 2.7E−02 | |
| 615 | PIGN | 2.7E−02 | |
| 616 | C12orf24 | 2.7E−02 | |
| 617 | C11orf51 | 2.7E−02 | |
| 618 | DRAM | 2.7E−02 | |
| 619 | CYP51A1 | 2.7E−02 | |
| 620 | ANXA1 | 2.7E−02 | |
| 621 | PLAUR | 2.7E−02 | 1 |
| 622 | SHQ1 | 2.7E−02 | |
| 623 | CD46 | 2.8E−02 | |
| 624 | RECQL | 2.8E−02 | |
| 625 | KMO | 2.8E−02 | |
| 626 | GUCA1A | 2.8E−02 | |
| 627 | PDK3 | 2.8E−02 | |
| 628 | PSMD9 | 2.8E−02 | |
| 629 | SPINK1 | 2.8E−02 | |
| 630 | UBE1C | 2.8E−02 | |
| 631 | MTERFD1 | 2.8E−02 | |
| 632 | RAGE | 2.8E−02 | |
| 633 | PVR | 2.8E−02 | |
| 634 | SLC35E3 | 2.8E−02 | |
| 635 | MMP12 | 2.9E−02 | |
| 636 | NRGN | 2.9E−02 | |
| 637 | CSDA | 2.9E−02 | |
| 638 | ATP6V1C1 | 2.9E−02 | |
| 639 | PIK3C2A | 2.9E−02 | |
| 640 | PSMB3 | 2.9E−02 | |
| 641 | FGA | 2.9E−02 | |
| 642 | PCGF1 | 2.9E−02 | |
| 643 | MRPL22 | 2.9E−02 | |
| 644 | SLC22A5 | 2.9E−02 | |
| 645 | HMOX1 | 2.9E−02 | |
| 646 | AQP1 | 2.9E−02 | |
| 647 | HR44 | 2.9E−02 | |
| 648 | CGRRF1 | 2.9E−02 | |
| 649 | PSMC2 | 2.9E−02 | |
| 650 | RMND5B | 2.9E−02 | |
| 651 | CRP | 2.9E−02 | |
| 652 | MRPL23 | 2.9E−02 | |
| 653 | PEX16 | 3.0E−02 | |
| 654 | GABRB2 | 3.0E−02 | |
| 655 | GBAS | 3.0E−02 | |
| 656 | DLC1 | 3.0E−02 | 1 |
| 657 | PPP2R1B | 3.0E−02 | |
| 658 | CAMK1 | 3.0E−02 | |
| 659 | SLC25A32 | 3.0E−02 | |
| 660 | SEPX1 | 3.0E−02 | |
| 661 | CDK10 | 3.0E−02 | |
| 662 | ADAM8 | 3.0E−02 | |
| 663 | MSN | 3.0E−02 | |
| 664 | PIR | 3.0E−02 | |
| 665 | PMM2 | 3.1E−02 | |
| 666 | PLA2G3 | 3.1E−02 | |
| 667 | MT1X | 3.1E−02 | |
| 668 | NEDD4L | 3.1E−02 | |
| 669 | ARPC2 | 3.1E−02 | |
| 670 | CD300A | 3.1E−02 | |
| 671 | ZCCHC10 | 3.1E−02 | |
| 672 | SLC3A1 | 3.1E−02 | |
| 673 | ABCA1 | 3.1E−02 | |
| 674 | ITGB5 | 3.1E−02 | |
| 675 | ASS1 | 3.1E−02 | |
| 676 | BCAT1 | 3.1E−02 | |
| 677 | POT1 | 3.1E−02 | |
| 678 | UBE2N | 3.2E−02 | |
| 679 | DARS | 3.2E−02 | |
| 680 | RINT1 | 3.2E−02 | |
| 681 | HSPB2 | 3.2E−02 | |
| 682 | NME5 | 3.2E−02 | |
| 683 | KIAA0101 | 3.2E−02 | |
| 684 | VAV3 | 3.2E−02 | |
| 685 | TMEM111 | 3.2E−02 | |
| 686 | MAX | 3.2E−02 | |
| 687 | PSMB2 | 3.2E−02 | |
| 688 | TAAR5 | 3.3E−02 | |
| 689 | PDHX | 3.3E−02 | |
| 690 | ZNF415 | 3.3E−02 | |
| 691 | SEC24A | 3.3E−02 | |
| 692 | CXCL5 | 3.3E−02 | |
| 693 | AMDHD2 | 3.3E−02 | |
| 694 | SPATA6 | 3.3E−02 | |
| 695 | C9orf3 | 3.3E−02 | |
| 696 | C1QBP | 3.3E−02 | |
| 697 | SEC24D | 3.3E−02 | |
| 698 | PSRC1 | 3.3E−02 | |
| 699 | LAMP2 | 3.3E−02 | |
| 700 | FKBP11 | 3.3E−02 | |
| 701 | LAMC1 | 3.3E−02 | |
| 702 | CASP1 | 3.3E−02 | |
| 703 | MCL1 | 3.3E−02 | |
| 704 | SLC35A2 | 3.3E−02 | |
| 705 | C2orf28 | 3.3E−02 | |
| 706 | HCCS | 3.4E−02 | |
| 707 | WDR61 | 3.4E−02 | |
| 708 | S100A14 | 3.4E−02 | |
| 709 | BDH1 | 3.4E−02 | |
| 710 | UFM1 | 3.5E−02 | |
| 711 | DKFZP586H2123 | 3.5E−02 | |
| 712 | CYP27A1 | 3.5E−02 | |
| 713 | NIT2 | 3.5E−02 | |
| 714 | CSGlcA-T | 3.5E−02 | |
| 715 | CD83 | 3.5E−02 | |
| 716 | GIP | 3.5E−02 | |
| 717 | DERL2 | 3.5E−02 | |
| 718 | MASP2 | 3.5E−02 | |
| 719 | PEX3 | 3.5E−02 | |
| 720 | NUPL1 | 3.5E−02 | |
| 721 | GSDMDC1 | 3.5E−02 | |
| 722 | PCK2 | 3.5E−02 | |
| 723 | TFAP2C | 3.6E−02 | |
| 724 | CLDN15 | 3.6E−02 | |
| 725 | KIAA1660 | 3.6E−02 | |
| 726 | PRMT3 | 3.6E−02 | |
| 727 | ECAT8 | 3.6E−02 | |
| 728 | MS4A2 | 3.6E−02 | |
| 729 | IFI35 | 3.6E−02 | |
| 730 | SLC31A1 | 3.6E−02 | |
| 731 | ASNS | 3.6E−02 | |
| 732 | NRL | 3.6E−02 | |
| 733 | PON2 | 3.6E−02 | |
| 734 | MPI | 3.6E−02 | |
| 735 | OAS1 | 3.6E−02 | |
| 736 | BAG2 | 3.6E−02 | |
| 737 | NUPR1 | 3.6E−02 | |
| 738 | SLC35A5 | 3.6E−02 | |
| 739 | NUDT15 | 3.6E−02 | |
| 740 | SDF2L1 | 3.6E−02 | |
| 741 | MDH2 | 3.6E−02 | |
| 742 | RER1 | 3.7E−02 | |
| 743 | SQRDL | 3.7E−02 | |
| 744 | SDS | 3.7E−02 | |
| 745 | SNX2 | 3.7E−02 | |
| 746 | FLJ20035 | 3.7E−02 | |
| 747 | NAGLU | 3.7E−02 | |
| 748 | TTC27 | 3.7E−02 | |
| 749 | TRIP6 | 3.7E−02 | |
| 750 | COPS8 | 3.7E−02 | |
| 751 | C21orf62 | 3.7E−02 | |
| 752 | FGF5 | 3.7E−02 | |
| 753 | TMEM168 | 3.7E−02 | |
| 754 | LEP | 3.7E−02 | |
| 755 | KIAA0692 | 3.7E−02 | |
| 756 | MIS12 | 3.7E−02 | |
| 757 | CCR4 | 3.7E−02 | |
| 758 | CCNB1 | 3.7E−02 | |
| 759 | C12orf47 | 3.8E−02 | |
| 760 | EMP1 | 3.8E−02 | 1 |
| 761 | APOBEC3F | 3.8E−02 | |
| 762 | GLB1 | 3.8E−02 | |
| 763 | CGA | 3.8E−02 | |
| 764 | SRPRB | 3.8E−02 | |
| 765 | KIAA0143 | 3.8E−02 | |
| 766 | NEK11 | 3.8E−02 | |
| 767 | REEP5 | 3.8E−02 | |
| 768 | NMI | 3.8E−02 | |
| 769 | CXCL14 | 3.8E−02 | |
| 770 | TUFT1 | 3.8E−02 | |
| 771 | ADAM7 | 3.8E−02 | |
| 772 | NUBP2 | 3.8E−02 | |
| 773 | NEDD9 | 3.8E−02 | |
| 774 | LMO4 | 3.9E−02 | |
| 775 | CTSB | 3.9E−02 | |
| 776 | KIAA0415 | 3.9E−02 | |
| 777 | TNFRSF1A | 3.9E−02 | |
| 778 | PRDX4 | 3.9E−02 | |
| 779 | HOXD11 | 3.9E−02 | |
| 780 | SH3BGR | 3.9E−02 | |
| 781 | CNGA3 | 3.9E−02 | |
| 782 | PHEX | 3.9E−02 | |
| 783 | CNIH4 | 4.0E−02 | |
| 784 | YKT6 | 4.0E−02 | |
| 785 | RWDD3 | 4.0E−02 | |
| 786 | AGTR1 | 4.0E−02 | |
| 787 | NRAS | 4.0E−02 | |
| 788 | SLC4A7 | 4.0E−02 | |
| 789 | CCDC53 | 4.0E−02 | |
| 790 | ZAK | 4.0E−02 | |
| 791 | DYNC1LI1 | 4.0E−02 | |
| 792 | AP2S1 | 4.1E−02 | |
| 793 | PIGL | 4.1E−02 | |
| 794 | C1RL | 4.1E−02 | 1 |
| 795 | SNAPC1 | 4.1E−02 | |
| 796 | HOXA2 | 4.1E−02 | |
| 797 | CNNM1 | 4.1E−02 | |
| 798 | RASAL1 | 4.1E−02 | |
| 799 | RGS12 | 4.1E−02 | |
| 800 | PAQR3 | 4.1E−02 | |
| 801 | HCG2P7 | 4.2E−02 | |
| 802 | DIABLO | 4.2E−02 | |
| 803 | CCT6A | 4.2E−02 | |
| 804 | SERPINE1 | 4.2E−02 | 1 |
| 805 | ETV5 | 4.2E−02 | |
| 806 | IDS | 4.2E−02 | |
| 807 | GSTM5 | 4.2E−02 | |
| 808 | TIMM44 | 4.2E−02 | |
| 809 | PTPRR | 4.2E−02 | |
| 810 | MEA1 | 4.2E−02 | |
| 811 | C1orf107 | 4.2E−02 | |
| 812 | XKR8 | 4.2E−02 | |
| 813 | PPL | 4.2E−02 | |
| 814 | MTHFS | 4.3E−02 | |
| 815 | PHLDB1 | 4.3E−02 | |
| 816 | PHLDA2 | 4.3E−02 | |
| 817 | SDF2 | 4.3E−02 | |
| 818 | LYRM1 | 4.3E−02 | |
| 819 | APOBEC3B | 4.3E−02 | |
| 820 | CASP7 | 4.3E−02 | |
| 821 | TM9SF1 | 4.3E−02 | |
| 822 | TAX1BP3 | 4.4E−02 | |
| 823 | LACTB2 | 4.4E−02 | |
| 824 | C9orf95 | 4.4E−02 | |
| 825 | TRIM36 | 4.4E−02 | |
| 826 | SIGLEC7 | 4.4E−02 | |
| 827 | SPRY1 | 4.4E−02 | |
| 828 | POLR2H | 4.4E−02 | |
| 829 | HTR5A | 4.4E−02 | |
| 830 | WNT11 | 4.4E−02 | |
| 831 | IL6ST | 4.4E−02 | |
| 832 | COMMD9 | 4.4E−02 | |
| 833 | FAM82B | 4.5E−02 | |
| 834 | MRPS18A | 4.5E−02 | |
| 835 | FBXO9 | 4.5E−02 | |
| 836 | IBSP | 4.5E−02 | |
| 837 | RPLP2 | 4.5E−02 | |
| 838 | NDUFB5 | 4.5E−02 | |
| 839 | RAB32 | 4.5E−02 | |
| 840 | PDLIM4 | 4.5E−02 | 1 |
| 841 | OXTR | 4.6E−02 | |
| 842 | MMP14 | 4.6E−02 | 1 |
| 843 | PSMB8 | 4.6E−02 | |
| 844 | LDLR | 4.6E−02 | |
| 845 | DUSP4 | 4.6E−02 | |
| 846 | CCDC72 | 4.6E−02 | |
| 847 | SS18L2 | 4.6E−02 | |
| 848 | PITX1 | 4.6E−02 | |
| 849 | LIF | 4.6E−02 | 1 |
| 850 | CRYBA2 | 4.6E−02 | |
| 851 | LRRC50 | 4.6E−02 | |
| 852 | SNX11 | 4.7E−02 | |
| 853 | RFNG | 4.7E−02 | |
| 854 | LAMP3 | 4.7E−02 | |
| 855 | EBAG9 | 4.7E−02 | |
| 856 | ABCA5 | 4.7E−02 | |
| 857 | KIAA0323 | 4.8E−02 | |
| 858 | ACTR1B | 4.8E−02 | |
| 859 | CDKN3 | 4.8E−02 | |
| 860 | CD1A | 4.8E−02 | |
| 861 | CSH1 | 4.8E−02 | |
| 862 | HOXC4 | 4.8E−02 | |
| 863 | SIPA1L1 | 4.8E−02 | |
| 864 | TMEM2 | 4.8E−02 | |
| 865 | CROT | 4.8E−02 | |
| 866 | PTDSS1 | 4.8E−02 | |
| 867 | HK3 | 4.8E−02 | 1 |
| 868 | SRPR | 4.8E−02 | |
| 869 | UCHL3 | 4.9E−02 | |
| 870 | ANXA4 | 4.9E−02 | |
| 871 | YIPF4 | 4.9E−02 | |
| 872 | TRIAP1 | 4.9E−02 | |
| 873 | ZFYVE21 | 4.9E−02 | |
| 874 | BST1 | 4.9E−02 | |
| 875 | SCN4A | 4.9E−02 | |
| 876 | IFI6 | 4.9E−02 | |
| 877 | WTAP | 4.9E−02 | |
| 878 | MBD4 | 5.0E−02 | |
| 879 | HOXD10 | 5.0E−02 | |
| 880 | LOH11CR2A | 5.0E−02 | |
| 881 | ZNF443 | 5.0E−02 | |
| 882 | CTR9 | 5.0E−02 | |
| 883 | HOP | 5.0E−02 | |
| 884 | CP | 5.0E−02 | |

TABLE 13
Table 13. MRs discovered by MRA and SLR using the TCGA data and TWPS signature.

| | MGES Analysis | | | | | TCGA Prognosis Analysis | | | |
|---|---|---|---|---|---|---|---|---|---|
| | MRA- | | | SLR- | LR- | MRA- | | | LR- |
| TF | rank | Overlap | P-value | rank | Coeff | rank | Overlap | P-value | Coeff |
| FOSL2 | 1 | 45 | 9.4E−39 | 5 | 0.21 | 4 | 69 | 1.9E−16 | 0.25 |
| ZNF238 | 2 | 37 | 9.6E−28 | 2 | −0.34 | — | — | — | — |
| RUNX1 | 3 | 37 | 2.3E−24 | 4 | 0.13 | — | — | — | — |
| C/EBP(*) | 4 | 30 | 3.2E−19 | 1 | 0.40 | 1 | 91 | 1.0E−28 | 0.42 |
| C/EBPδ | 5 | 27 | 1.2E−19 | 6 | 0.42 | 3 | 75 | 1.8E−27 | 0.41 |
| STAT3 | 6 | 26 | 1.2E−16 | 7 | 0.40 | 7 | 60 | 9.4E−17 | 0.21 |
| BHLHB2 | 7 | 25 | 7.8E−21 | 9 | 0.41 | 2 | 78 | 5.3E−41 | 0.36 |
| MYCN | 8 | 25 | 6.2E−20 | 37 | −0.11 | — | — | — | — |
| FOSL1 | 9 | 23 | 3.6E−25 | 47 | 0.24 | 19 | 30 | 1.0E−11 | 0.28 |
| ELF4 | 10 | 21 | 7.0E−09 | 34 | 0.1 | — | — | — | |
| C/EBPβ | 11 | 20 | 2.2E−15 | 28 | 0.35 | 10 | 45 | 1.5E−13 | 0.44 |
| LZTS1 | 12 | 20 | 3.8E−14 | 3 | 0.22 | — | — | — | — |
| TBX2 | 13 | 17 | 4.6E−12 | 23 | 0.17 | 21 | 28 | 1.6E−06 | 0.13 |
| SATB1 | 14 | 17 | 1.4E−07 | 21 | −0.32 | — | — | — | — |
| IRF1 | 15 | 16 | 2.0E−11 | 19 | 0.48 | — | — | — | — |
| EPAS1 | 16 | 16 | 2.6E−09 | 16 | 0.21 | — | — | — | — |
| NFIB | 17 | 15 | 5.4E−07 | 8 | −0.32 | — | — | — | — |
| KLF6 | 18 | 14 | 2.0E−11 | — | 0.16 | — | — | — | — |
| NFYB | 19 | 14 | 3.5E−07 | 14 | −0.55 | — | — | — | — |
| ELK3 | 20 | 14 | 1.8E−06 | 53 | 0.24 | 14 | 35 | 2.1E−05 | 0.19 |

‘—’ indicate that TF is not significant in regulon enrichment analysis and not included in SLR analysis
(*)The C/EBP metagene includes targets of both C/EBPβ and C/EBPδ

TABLE 14
Immunohistochemistry results of GBM tumor specimens for C/EBPβ
and p-Stat3 and comparison with YKL-40 expression.

| | STAT3− | STAT3+ |
|---|---|---|
| YKL40− | 12 | 2 |
| YKL40+ | 14 | 34 |
| FET | 0.00022 | |
| | CEBPB− | CEBPB+ |
| YKL40− | 9 | 5 |
| YKL40+ | 4 | 44 |
| FET | 4.9E−05 | |
| | DOUBLE− | DOUBLE+ |
| YKL40− | 8 | 1 |
| YKL40+ | 2 | 32 |
| FET | 2.7E−06 | |

Tumors were scored as positive or negative as described in the Methods herein. Expression of either C/EBPβ or STAT3 was significantly associated with YKL40 expression (C/EBPβ, P=4.9×10⁻⁵; STAT3, P=2.2×10⁻⁴), with higher association in double-positive tumours (C/EBPβ⁺ STAT3⁺, P=2.7×10⁻⁶) versus double-negative ones (C/EBPβ⁻ STAT3⁻, Table 14).

TABLE 15
Primers used for ChIP assays.

| | | SEQ |
|---|---|---|
| | | ID |
| ChIP_Stat3 | Primers | NO: |
| Rrpb1_3655_f1 | ATCTGGATGGCATTTTCAGG | 5 |
| Rrpb1_3801_r1 | GGGGTAACATTCGCAGTTGT | 6 |
| Serpinh1_3546 | CCTCACCATCTCTCCTTTGC | 7 |
| Serpinh1_3677_r | GGGTCCCAAACACTTGAGAG | 8 |
| Chi3I1_3311_f | CTGAGGTCTCTTGCCGAATC | 9 |
| Chi3I1_3511_r | TGTCGATGTGATCGTTGCTT | 10 |
| Timp1_2035_f | GGTGGGTGGATGAGTAATGC | 11 |
| Timp1_2194_r | CCCTGCTTACCTCTGGTGTC | 12 |
| Socs3_f_1876_f | GCGCTCAGCCTTTCTCTG | 13 |
| Socs3_r_2025_r | GGAGCAGGGAGTCCAAGTC | 14 |
| Osmr_3468_f | TGGGTGGGGTGTTTCATTAT | 15 |
| Osmr_3666_r | GAACAAATGCTACGGGGAAA | 16 |
| Actn1_1054_f | TAGATCACTCGGGGTTGTCC | 17 |
| Actn1_1290_r | ACTGCTCTCAGAGGCTACCG | 18 |
| Slc16a3_3601_ | CCAGTGAGGTGCCAAATGT | 19 |
| Slc16a3_3731_r | GACGCCCTGAGCTCTGTCT | 20 |
| Col4a1_2620_f | TTTGGGCGTATTTCTCCTTG | 21 |
| Col4a1_2806_r | AGAAGGCAACGAGTTGAGGA | 22 |
| Col4a2_1023_f | AGAAGGCAACGAGTTGAGGA | 23 |
| Col4a2_1209_r | TTTGGGCGTATTTCTCCTTG | 24 |
| Itga7_3019_f | GCAGCAGCTGTAGCAGTGAG | 25 |
| Itga7_3246_r | GCCAAGGATACAGGCAACAT | 26 |
| Cd151_3821 | AGGGGCATAGCCTGTCTGT | 27 |
| Cd151_3983_r | CAGGCCTGTTTACGGTCTGT | 28 |
| Icam1_365_f | CCCAGGTGGATTTTTGTCTG | 29 |
| Icam1_488_r | ACAATGGTGCCGTTCTTTTC | 30 |
| Runx1_2719_f | TGCGAGTAAGTTGTGCTGGT | 31 |
| Runx1-2849_r | CAGCATGCCGAGTTAAGGAT | 32 |
| Bhlhb2_1160_f | TTCCCATGGGGTGACATC | 33 |
| Bhlhb2_1277_r | CAGAGGCTGGGGTTTCTTTC | 34 |
| Fosl2_275_f | TGACCCCGAGTATTGTTTGG | 35 |
| Fosl2_423_r | GGGGTGTTGGTAGCAGAGAA | 36 |
| Stat3_1501_f | CAGGAGGGAGCTGTATCAGG | 37 |
| Stat3_1630_r | AGGACTTGGGCACAGAAGC | 38 |
| ChIP_CEBPβ | Primers | |
| Ptrf_1587_f | GCAAGGGTCCTTTTGTGCT | 39 |
| Ptrf_1700_r | GCTCATCCGAAAATCCTCAA | 40 |
| Shc1_1367_f2 | CGCAACCACTTTGTTTTACG | 41 |
| Shc1_1514_r2 | GCTGAGGGCACAAGGAATTA | 42 |
| Mvp_3222_f2 | CGGCTCCGTCCTTTGATAAC | 43 |
| Mvp_3354_r2 | AGCTCCCACTTCAGATGAGC | 44 |
| Serpine1_720_f | GGGCTCCCACTGATTCTACA | 45 |
| Serpine1_843_r | ATGGTTTCGGGATGATTCAA | 46 |
| Timp1_2314_f2 | GGGCTAGTCTAGGGGGAAGA | 47 |
| Timp1_2390_r2 | GGGGTTCTAGGGAGTTTGGA | 48 |
| Serpina1_914_f4 | TGTGCTGTCATCCAGAGTTTG | 49 |
| Serpina1_1057_r4 | GGGTCTAGTGCTGCTGATGA | 50 |
| S100a11_2551_f | CATTGGCTCTCCACACCAG | 51 |
| S100a11_2642_r | ACATGTGTGTGCATGTGCTG | 52 |
| Slc26a3_2504_f | CGCAACACCCTGAACACTC | 53 |
| Slc26a3_2580_r | CACTTCCCTGCACGGTCT | 54 |
| Myl9_502_f | TGGGATAACTGGCACAACCT | 55 |
| Myl9_571_r | TCAGGACAATTTTCACATTGATT | 56 |
| Stat3_2639_f2 | CTGGCTGGTCGTGGGTAG | 57 |
| Stat3_2755_r2 | GGGAGCATAATTTAACCTAGAAAAAG | 58 |
| Cebpb_401_f | ACCCCAGCTCAGCAGATAAC | 59 |
| Cebpb_450_r | ACCTCTCTGCCACTCCTAGC | 60 |
| Fosl2_516_f1 | TCCTCATAAGGACCCTGTGG | 61 |
| Fosl2_626_r1 | TGTAGCGGAAGTCAGGGAAC | 62 |
| Runx1_1362_f | AAGTTGTCCATTTAGGGGGAAT | 63 |
| Bhlhb2_434_f1 | TGGCCTCGATACAATTTTCC | 69 |
| Bhlhb2_555_r1 | TAGGCGCTGCACTAGTTGAT | 65 |
| ChIP_FosL2 | Primers | |
| Actn1(2)_3581_f | CAGCCAAAGGCATCCTGTAT | 66 |
| Actn1(2)_3711_r | GGTCATCCTGCTTTGAGGAA | 67 |
| Itga5(1)_1894_f | GCGGGCTCAGAGTTCCAG | 68 |
| Itga5(1)2027_r | CGCTTCCTAAACCTCCCAGA | 69 |
| Socs3_1734_f | CCTTCGAACTTGCTTTGCAT | 70 |
| Socs3_1816_r | GCAGCCACCTAGACTTACCG | 71 |
| S100a11(1)_1185_f | CTCCGGGACACCTGTGTATT | 72 |
| S100a11(1)_1308_r | CTGAGGAGTGGATGCATGTG | 73 |
| c1r(3)_3178_f | ACTGAGGGGAGAAGCACAGA | 74 |
| c1r(3)_3308_r | AAGCTGAGGCACAGTGGTTT | 75 |
| Flna(2)_3196_f | CACCCACCTCCTGACACTCT | 76 |
| Flna(2)_3295_r | CTGGGTTGTCTGGGTTCATT | 77 |
| Tagln_1202_f | TATTGACACTGCCCACTGGA | 78 |
| Tagln_1351_r | CACCCTTTCAATTGGACCAC | 79 |
| Emp3_2706_f | TCCCTGGTGCTTAGAGATGG | 80 |
| Emp3_2848_r | CCGACATCAGGATTGAGGAG | 81 |
| Plau_278_f | TTGGCTCTGAAGCCTATAGCA | 82 |
| Plau_420_r | CCTGCTGGGGAAAGTACAAG | 83 |
| Thbd(1)_38_f | AACGAGGTTCCTGCCCTTAT | 84 |
| Thbd(1)_180_r | AGTCAAGCTGTGGCTGCCTA | 85 |
| Tnc(2)_1060_f | ACTCCCTTAAATGCCCCTGT | 86 |
| Tnc(2)_1180_r | ATAAGCTGCGCCTTTGCTT | 87 |
| Acta2_135_f | AAAATTCACAGGGCTGTTGC | 88 |
| Acta2_580_r | TCTCTGGCCCTGTAACTTGC | 89 |
| Ehd2_645_f2 | AGGGGAGAGAGTGAGGCATT | 90 |
| Ehd2_814_r2 | CCACCTACATCTCCCCTGTC | 91 |
| Bace2(2)_1027_f | CAGCTGGAGGAGGTACAAAGA | 92 |
| Bace2(2)_1175_r | GCCAAGACGCAGAAATGC | 93 |
| Slc16a3_282_f | GGCAGATGTGGAAGGTGTCT | 94 |
| Slc16a3_415_r | GGGTCCCCTATGGGGTATT | 95 |
| Runx1_3473_f5 | TGATGGTTTGGCAAAGCTG | 96 |
| Runx1_3609_r5 | GCATTCCCCTGCTCACTTAG | 97 |
| Stat3_3395_f3 | TTGGTTCAGCCAGTTTTCTATC | 98 |
| Stat3_3544_r3 | TCCAGACTTGTTTCCCCATC | 99 |
| Cebpb_1129_f1 | GATTGCAGCTGGGAGAAGTG | 100 |
| Cebpb_1278_r1 | CTGCTCGAGGCTTGGACAC | 101 |
| Fosl2_434_f1 | CCACCCCCAGTTTTCTGAG | 102 |
| Fosl2_551_r1 | GGCTTGCCTGGGTGTTTAC | 103 |
| Bhlhb2_1361_f2 | GGGCTGGAGCTAGCAAGG | 104 |
| Bhlhb2_1503_r2 | AGGGGGAGAAGTTGGTAACG | 105 |
| ChIP_b-HLH-B2 | Primers | |
| Serpine1_1227_f | TCAGGGGCACAGAGAGAGTC | 106 |
| Serpine1_1375_r | CAGCCACGTGATTGTCTAGG | 107 |
| Efemp2_885_f | ATGGTGGTGGCAGAGTGG | 108 |
| Efemp2_1027_r | CTGCTTATCCCCGCAGTC | 109 |
| Slc16a3_3151_f | GGAGGGAAGGAACTGAGGAG | 110 |
| Slc16a3_3290_r | ACCCCAGACTCTGTCCACAC | 111 |
| Bcl3_1175_f | AGCCCCTTTAGACCCACAG | 112 |
| Bcl3_1318_r | AGCCGTTTCCTCCTTAGTGG | 113 |
| Pdpn_2946_f | GCTTCCGAGGAGTGTGAGTG | 114 |
| Pdpn_3046_r | CACTGATGTTGTTGCCCAAG | 115 |
| Ifitm3_3298_f | GAGCCGAGTCCTGTATCAGC | 116 |
| Ifitm3_3443_r | CCTGCTCAGTCTCAGAACCAC | 117 |
| Flna_3577_f | GCACCCCCTAACACCACTAC | 118 |
| Flna_3718_r | CATGCCCAAATATGGTTGAC | 119 |
| Fcgr2c_112_f | GCCAATTTACCGAGAGCAAG | 120 |
| Fcgr2c_214_r | TGGAGGGGAAAGAGGAAGAG | 121 |
| Socs3_1058_f | ACCTCCCTGAACCTGAGTTG | 122 |
| Socs3_1207_r | ACAAGGCAGGCATTCTCATC | 123 |
| Slc39a8_523_f | CCTGATGAAAGGCAAGAACG | 124 |
| Slc39a8_1030_r | GGACTTCCTGAGGCTGTGTC | 125 |
| Lif_82_f | CCTGGTCACATGGATTTGG | 126 |
| Lif_219_r | ATCTCCTGCACAAGGACCTG | 127 |
| Runx1_674_f | TTTCTGAAGTGCCTGTGCTG | 128 |
| Runx1_815_r | GCTCTGCTCTGCCTACATCC | 129 |
| Stat3_1376_f | AGGAGTTGGGTCCCCAGAG | 130 |
| Stat3_1520_r | CCTGATACAGCTCCCTCCTG | 131 |
| Cebpb_3549_f2 | TTTCGAAGTTGATGCAATCG | 132 |
| Cebpb_3673_r | AACAAGCCCGTAGGAACATC | 133 |
| Olr_f | ACTGCACCTGGCCAACTTTT | 134 |
| Olr_r | TGCAAAGAAAAGAATACACAAAGGA | 135 |

TABLE 16
Primers used for qRT-PCR.

| Primers | | SEQ |
|---|---|---|
| mesenchymal genes | | ID |
| mouse | Sequence (5′-3′) | NO: |
| mSerpinh1_f | GCCGAGGTGAAGAAACCCC | 136 |
| mSerpinh1_r | CATCGCCTGATATAGGCTGAAG | 137 |
| mCol4a1f | CCAGGTGAAAGGGGAGAAAAAG | 138 |
| mCol4a1_r | CCAGGTTGACACTCCACAATG | 139 |
| mPlau_f | CCTTCAGAAACCCTACAATGCC | 140 |
| mPlau_r | CAAACTGCCTTAGGCCAATCT | 141 |
| mActa2f | GGACGTACAACTGGTATTGTGC | 142 |
| mActa2_r | CGGCAGTAGTCACGAAGGAAT | 193 |
| mSocs3_f | TGCGCCTCAAGACCTTCAG | 144 |
| mSocs3_r | GAGCTGTCGCGGATCAGAAA | 195 |
| mSerpine1_f | CATCCCCCATCCTACGTGG | 146 |
| mSerpine1_r | CCCCATAGGGTGAGAAAACCA | 147 |
| mItga7_qPCR_F1 | CTGCTGTGGAAGCTGGGATTC | 148 |
| mItga7_qPCR_R1 | CTCCTCCTTGAACTGCTGTCG | 149 |
| mOsmr_qPCR_f1 | CATCCCGAAGCGAAGTCTTGG | 150 |
| mOsmr_qPCR_r1 | GGCTGGGACAGTCCATTCTAAA | 151 |
| mTimpl_qPCR_f1 | CTTGGTTCCCTGGCGTACTC | 152 |
| mTimpl_qPCR_r1 | ACCTGATCCGTCCACAAACAG | 153 |
| mPlaur_qPCR_f1 | CAGAGCTTTCCACCGAATGG | 154 |
| mPlaur_qPCR_r1 | GTCCCCGGCAGTTGATGAG | 155 |
| mGapdh_f | TGACCACAGTCCATGCCATC | 156 |
| mGapdh_r | GACGGACACATTGGGGGTAG | 157 |
| mCtgf_f | GGGCCTCTTCTGCGATTTC | 158 |
| mCtgf_r | ATCCAGGCAAGTGCATTGGTA | 159 |
| mFibfonectin_f | GCAGTGACCACCATTCCTG | 160 |
| mFibronectin_r | GGTAGCCAGTGAGCTGAACAC | 161 |
| mCyr61_f | CTGCGCTAAACAACTCAACGA | 162 |
| mCyr61_r | GCAGATCCCTTTCAGAGCGG | 163 |
| mSparc_f | GTGGAAATGGGAGAATTTGAGGA | 164 |
| mSparc_r | CTCACACACCTTGCCATGTTT | 165 |
| mActn1_f | GACCATTATGATTCCCAGCAGAC | 166 |
| mActn1_r | CGGAAGTCCTCTTCGATGTTCTC | 167 |
| mBace2_f | GGAGCCTGTCAGGGCTACT | 168 |
| mBace2_r | CCACAAGAATCTGTACCTTCTGC | 169 |
| mGfap_f | CGGAGACGCATCACCTCTG | 170 |
| mGfap_r | AGGGAGTGGAGGAGTCATTCG | 171 |
| mDoublecortin_f | AAACTGGAAACCGGAGTTGTC | 172 |
| m_Doublecortin_r | CGTCTTGGTCGTTACCTGAGT | 173 |
| m_Olig2_f | CTGGTGTCTAGTCGCCCATC | 174 |
| m_Olig2_r | GGGCTCAGTCATCTGCTTCT | 175 |
| mBetaIIITubulin_f | TGGACAGTGTTCGGTCTGG | 176 |
| mBetaIIITubulin_r | CCTCCGTATAGTGCCCTTTGG | 177 |
| rCebpb_f | ATCGACTTCAGCCCCTACCT | 178 |
| rCebpb_r | GGCTCACGTAACCGTAGTCG | 179 |
| m18s_f | TCAAGAACGAAAGTCGGAGG | 180 |
| m18s_r | GGACATCTAAGGGCATCACA | 181 |
| mStat3_f | TGGCACCTTGGATTGAGAGTC | 182 |
| mStat3_r | GCAGGAATCGGCTATATTGCT | 183 |
| mChi3I1_f | GTACAAGCTGGTCTGCTACTTC | 184 |
| mChi3I1_r | ATGTGCTAAGCATGTTGTCGC | 185 |
| mβActin_f | GATGACGATATCGCTGCGCTG | 186 |
| mβActin_f | GTACGACCAGAGGCATACAGG | 187 |
| Primers | | |
| mesenchymal genes | | |
| human | Sequence (5′-3′) | |
| hSOCS3_f | GAGCTGTCGCGGATCAGAAA | 188 |
| hSOCS3_r | TGACCAACATTGATAGCTCAGAC | 189 |
| hlTGA7_f | GCGCAGGATAACCACAGCA | 190 |
| hlTGA7_r | AGGATTGAAACATCCAATGTCA | 191 |
| hOSMR_f | GCTCCAGAAATTTGGCTCAG | 192 |
| hOSMR_r | CCACCCTAATCAAGGAAATGA | 193 |
| hCHl3L1_f | TGAAATCCAGGTGTTGGGATA | 194 |
| hCHl3L1_r | TCAAGATGACCAAGATGTATAAAGG | 195 |
| hTIMP1_f | GCAGTTTTCCAGCAATGAGA | 196 |
| hTIMP1_r | CTGACATTCCCAAGGAGGAG | 197 |
| hSTAT3_136_f | AGGTGAGGGACTCAAACTGC | 198 |
| hSTAT3_331_r | ATCGACTTCAGCCCGTACC | 199 |
| hCEBP13_412 _f | CCGTAGTCGTCGGAGAAGAG | 200 |
| hCEBP13_575_r | CGCCGCTAGAGGTGAAATTC | 201 |
| h18s-f | CGCCGCTAGAGGTGAAATTC | 202 |
| h18s-r | CTTTCGCTCTGGTCCGTCTT | 203 |
| hCOL1A2_f | TCTGGATGGATTGAAGGGACA | 204 |
| hCOL1A2_r | CCAACACGTCCTCTCTCACC | 205 |
| hFN_f | GAAGGCTTGAACCAACCTACG | 206 |
| hFN_r | TGATTCAGACATTCGTTCCCAC | 207 |
| hCDH11_f | TCCCAGGGAAGACATGAGATT | 208 |
| hCDH11_r | TGTAGCCACCACATAGAGGAA | 209 |
| hTNC_f | GCACACAGTAGATGGGGAAAA | 210 |
| hTNC_r | CAGCAGCTCCTTAACATCAGG | 211 |
| hIGFBP5_f | TGTGACCGCAAAGGATTCTAC | 212 |
| hIGFBP5_r | GCAGCTTCATCCCGTACTTG | 213 |
| hCOL5A1_f | GCATTTCCCGAGGACTTCTCC | 214 |
| hCOL5A1_r | AATCTGCTGGATACCCTGCTC | 215 |
| hFOSL2_f | TATCCCGGGAACTTTGACAC | 216 |
| hFOSL2_r | TGAGCCAGGCATATCTACCC | 217 |
| hBHLHB2_f | CAGCAGCAGAAAATCATTGC | 218 |
| hBHLHB2_r | TTCAGGTCCCGAGTGTTCTC | 219 |
| hRUNX1_f | CCCATCGCTTTCAAGGTG | 220 |
| hRUNX1_r | TGGTCAGAGTGAAGCTTTTCC | 221 |
| hCTGF_f | GGCAAAAAGTGCATCCGTACT | 222 |
| hCTGF_r | CCGTCGGTACATACTCCACAG | 223 |

TABLE 17
shRNA sequences

| Gene | Gene ID | TRC number | Clone ID |
|---|---|---|---|
| Stat3 | 6774 | TRCN0000020843 | NM_003150.2-361s1c1 |
| SEQ ID | CCGGGCAAAGAATCACATGCCACTTCTCGAGAAGTGGCATGTGATTCTTTGCTTTTT | | |
| NO: 224 | | | |
| C/EBPβ | 1051 | TRCN0000007442 | NM_005194.2-540s1c1 |
| SEQ ID | CCGGCGACTTCCTCTCCGACCTCTTCTCGAGAAGAGGTCGGAGAGGAAGTCGTTTTT | | |
| NO: 225 | | | |
| Fosl2 | 2355 | TRCN0000016142 | NM_005253.3-1368s1c1 |
| SEQ ID | CCGGCACGGCCCAGTGTGCAAGATTCTCGAGAATCTTGCACACTGGGCCGTGTTTTT | | |
| NO: 226 | | | |
| bHLHB2 | 8553 | TRCN0000013249 | NM_003670.1-512s1c1 |
| SEQ ID | CCGGGCACTAACAAACCTAATTGATCTCGAGATCAATTAGGTTTGTTAGTGCTTTTT | | |
| NO: 227 | | | |
| Runx1 | 861 | TRCN0000013660 | NM_001754.2-1051s1c1 |

| | |
|---|---|
| SEQ ID | CCGGCCTCGAAGACATCGGCAGAAACTCGAGTTTCTGCCGATGTCTTCGAGGTTTTT |
| NO: 228 | |

Aoki, K., Meng, G., Suzuki, K., Takashi, T., Kameoka, Y., Nakahara, K., lshida, R., and Kasai, M. (1998). RP58 associates with condensed chromatin and mediates a sequence-specific transcriptional repression. J Biol Chem 273, 26698-26704.
Ashburner, M., Ball, C. A., Blake, J. A., Botstein, D., Butler, H., Chemy, J. M., Davis, A. P., Dolinski, K., Dwight, S. S., Eppig, J. T., et al. (2000). Gene ontology: tool for the unification of biology. The Gene Ontology Consortium. Nat. Genet. 25, 25-29.
Bachoo, R. M., Maher, E. A., Ligon, K. L., Sharpless, N. E., Chan, S. S., You, M. J., Tang, Y., DeFrances, J., Stover, E., Weissleder, R., et al. (2002). Epidermal growth factor receptor and Ink4a/Arf: convergent mechanisms governing terminal differentiation and transformation along the neural stem cell to astrocyte axis. Cancer Cell 1,269-277.
Barnabe-Heider, F., Wasylnka, J. A., Fernandes, K. J., Porsche, C., Sendtner, M., Kaplan, D. R., and Miller, F. D. (2005). Evidence that embryonic neurons regulate the onset of cortical gliogenesis via cardiotrophin-1. Neuron 48, 253-265.
Basso, K., Margolin, A. A., Stolovitzky, G., Klein, U., Dalla-Favera, R., and Califano, A. (2005). Reverse engineering of regulatory networks in human B cells. Nat Genet. 37, 382-390.
Bonni, A., Sun, Y., Nadal-Vicens, M., Bhatt, A., Frank, D. A., Rozovsky, 1., Stahl, N., Yancopoulos, G. D., and Greenberg, M. E. (1997). Regulation of gliogenesis in the central nervous system by the JAK-STAT signaling pathway. Science 278, 477-483.
Bromberg, J. F., Wrzeszczynska, M. H., Devgan, G., Zhao, Y., Pestell, R. G., Albanese, C., and Darnell, J. E., Jr. (1999). Stat3 as an oncogene. Cell 98, 295-303.
Bussemaker, H. J., L1, H., and Siggia, E. D. (2001). Regulatory element detection using correlation with expression. Nat Genet. 27, 167-171.
Demuth, T., and Berens, M. E. (2004). Molecular mechanisms of glioma cell migration and invasion. J Neurooncol 70, 217-228.
Frank, S. R., Schroeder, M., Fernandez, P., Taubert, S., and Amati, B. (2001). Binding of c-Myc to chromatin mediates mitogen-induced acetylation of histone H4 and gene activation. Genes Dev 15, 2069-2082.
Freije, W. A., Castro-Vargas, F. E., Fang, Z., Horvath, S., Cloughesy, T., Liau, L. M., Mischel, P. S., and Nelson, S. F. (2004). Gene expression profiling of gliomas strongly predicts survival. Cancer Res 64, 6503-6510.
Fuks, F., Burgers, W. A., Godin, N., Kasai, M., and Kouzarides, T. (2001). Dnmt3a binds deacetylases and is recruited by a sequence-specific repressor to silence transcription. Embo J 20, 2536-2544.
Hanauer, D. A., Rhodes, D. R., Sinha-Kumar, C., and Chinnaiyan, A. M. (2007). Bioinformatics approaches in the study of cancer. Curr Mol Med 7, 133-141.
He, F., Ge, W., Martinowich, K., Becker-Catania, S., Coskun, V., Zhu, W., Wu, H., Castro, D., Guillemot, F., Fan, G., et al. (2005). A positive autoregulatory loop of Jak-STAT signaling controls the onset of astrogliogenesis. Nat Neurosci 8, 616-625.
Hoelzinger, D. B., Demuth, T., and Berens, M. E. (2007). Autocrine factors that sustain glioma invasion and paracrine biology in the brain microenvironment. J Natl Cancer Inst 99, 1583-1593.
Kalir, S., Mangan, S., and Alon, U. (2005). A coherent feed-forward loop with a SUM input function prolongs flagella expression in Escherichia coli. Mol Syst Biol 1, 2005 0006.
Kargiotis, 0., Rao, J. S., and Kyritsis, A. P. (2006). Mechanisms of angiogenesis in gliomas. J Neurooncol 78, 281-293.
Lander, A. D. (2004). A calculus of purpose. PLoS Biol 2, e164.
Lee, J., Kotliarova, S., Kotliarov, Y., L1, A., Su, Q., Donin, N. M., Pastorino, S., Purow, B. W., Christopher, N., Zhang, W., et al. (2006). Tumor stem cells derived from glioblastomas cultured in bFGF and EGF more closely mirror the phenotype and genotype of primary tumors than do serum-cultured cell lines. Cancer Cell 9, 391-403.
Lee, J. P., Jeyakumar, M., Gonzalez, R., Takahashi, H., Lee, P. J., Baek, R. C., Clark, D., Rose, H., Fu, G., Clarke, J., et al (2007). Stem cells act through multiple mechanisms to benefit mice with neurodegenerative metabolic disease. Nat Med 13, 439-447.
Mani, K. M., Lefebvre, C., Wang, K., Lim, W. K., Basso, K., Dalla Favera, R., and Califano, A. (2007). A Systems biology approach to prediction of oncogenes and perturbation targets in B cell lymphomas. Molecular Systems Biology in press.
Margolin, A. A., Nemenman, I., Basso, K., Wiggins, C., Stolovitzky, G., Dalla Favera, R., and Califano, A. (2006a). ARACNE: an algorithm for the reconstruction of gene regulatory networks in a mammalian cellular context. BMC Bioinformatics 7 Suppl 1, S7.
Margolin, A. A., Wang, K., Lim, W. K., Kustagi, M., Nemenman, I., and Califano, A. (2006b). Reverse engineering cellular networks. Nat Protoc 1, 662-671.
Menard, C., Hein, P., Paquin, A., Savelson, A., Yang, X. M., Lederfein, D., Barnabe-Heider, F., Mir, A. A., Stemeck, E., Peterson, A. C., et al. (2002). An essential role for a MEK-C/EBP pathway during growth factor-regulated cortical neurogenesis. Neuron 36, 597-610.
Nadeau, S., Hein, P., Fernandes, K. J., Peterson, A. C., and Miller, F. D. (2005). A transcriptional role for C/EBP beta in the neuronal response to axonal injury. Mol Cell Neurosci 29, 525-535.
Nakashima, K., Yanagisawa, M., Arakawa, H., Kimura, N., Hisatsune, T., Kawabata, M., Miyazono, K., and Taga, T. (1999). Synergistic signaling in fetal brain by STAT3-Smadl complex bridged by p300. Science 284, 479-482.
Network, A. (2008). Comprehensive genomic characterization defines human glioblastoma genes and core pathways. Nature 455, 1061-1068.
Niehof, M., Kubicka, S., Zender, L., Manns, M. P., and Trautwein, C. (2001). Autoregulation enables different pathways to control CCAAT/enhancer binding protein beta (C/EBP beta) transcription. J Mol Biol 309, 855-868.
Nigro, J. M., Misra, A., Zhang, L., Smirnov, I., Colman, H., Griffin, C., Ozburn, N., Chen, M., Pan, E., Koul, D., et al. (2005). Integrated array-comparative genomic hybridization and expression array profiles identify clinically relevant molecular subtypes of glioblastoma. Cancer Res 65, 1678-1686.
Ohgaki, H., and Kleihues, P. (2005). Population-based studies on incidence, survival rates, and genetic alterations in astrocytic and oligodendroglial gliomas. J Neuropathol Exp Neurol 64, 479-489.
Palomero, T., Lim, W. K., Odom, D. T., Sulis, M. L., Real, P. J., Margolin, A., Barnes, K. C., O'Neil, J., Neuberg, D., Weng, A. P., et al. (2006). NOTCH I directly regulates c-MYC and activates a feed-forward-loop transcriptional network promoting leukemic cell growth. Proc Natl Acad Sci USA 103, 18261-18266.
Paquin, A., Barnabe-Heider, F., Kageyama, R., and Miller, F. D. (2005). CCAAT/enhancer-binding protein phosphorylation biases cortical precursors to generate neurons rather than astrocytes in vivo. J Neurosci 25, 10747-10758.
Park, K. I., Hack, M. A., Ourednik, J., Yandava, B., Flax, J. D., Stieg, P. E., Gullans, S., Jensen, F. E., Sidman, R. L., Ourednik, V., et al. (2006). Acute injury directs the migration, proliferation, and differentiation of solid organ stem cells: evidence from the effect of hypoxia-ischemia in the CNS on clonal “reporter” neural stem cells. Exp Neurol 199, 156-178.
Parker, M. A., Anderson, J. K., Corliss, D. A., Abraria, V. E., Sidman, R. L., Park, K. I., Teng, Y. D., Cotanche, D. A., and Snyder, E. Y. (2005). Expression profile of an operationally-defined neural stem cell clone. Exp Neurol 194, 320-332.
Pelloski, C. E., Mahajan, A., Maor, M., Chang, E. L., Woo, S., Gilbert, M., Colman, H., Yang, H., Ledoux, A., Blair, H., et al. (2005). YKL-40 expression is associated with poorer response to radiation and shorter overall survival in glioblastoma. Clin Cancer Res 11, 3326-3334.
Phillips, H. S., Kharbanda, S., Chen, R., Forrest, W. F., Soriano, R. H., Wu, T. D., Misra, A., Nigro, J. M., Colman, H., Soroceanu, L., et al. (2006). Molecular subclasses of high-grade glioma predict prognosis, delineate a pattern of disease progression, and resemble stages in neurogenesis. Cancer Cell 9, 157-173.
Ramji, D. P., and Foka, P. (2002). CCAAT/enhancer-binding proteins: structure, function and regulation. Biochem J 365, 561-575.
Rhodes, D. R., and Chinnaiyan, A. M. (2005). Integrative analysis of the cancer transcriptome. Nat Genet. 37 Suppl, S31-37.
Rothschild, G., Zhao, X., Iavarone, A., and Lasorella, A. (2006). E Proteins and ld2 Converge on p57Kip2 To Regulate Cell Cycle in Neural Cells. Mol Cell Biol 26, 4351-4361.
Simmons, M. L., Lamborn, K. R., Takahashi, M., Chen, P., Israel, M. A., Berger, M. S., Godfrey, T., Nigro, J., Prados, M., Chang, S., et al. (2001). Analysis of complex relationships between age, p53, epidermal growth factor receptor, and survival in glioblastoma patients. Cancer Res 61, 1122-1128.
Sterneck, E., and Johnson, P. F. (1998). CCAAT/enhancer binding protein beta is a neuronal transcriptional regulator activated by nerve growth factor receptor signaling. J Neurochem 70, 2424-2433.
Takashima, Y., Era, T., Nakao, K., Kondo, S., Kasuga, M., Smith, A. G., and Nishikawa, S. (2007). Neuroepithelial cells supply an initial transient wave of MSC differentiation. Cell 129, 1377-1388.
Tarin, D., Thompson, E. W., and Newgreen, D. F. (2005). The fallacy of epithelial mesenchymal transition in neoplasia. Cancer Res 65, 5996-6000; discussion 6000-5991.
Tegner, J., Yeung, M. K., Hasty, J., and Collins, J. J. (2003). Reverse engineering gene networks: integrating genetic perturbations with dynamical modeling. Proc Natl Acad Sci USA 100, 5944-5949.
Tso, C. L., Shintaku, P., Chen, J., Liu, Q., Liu, J., Chen, Z., Yoshimoto, K., Mischel, P. S., Cloughesy, T. F., Liau, L. M., et al. (2006). Primary glioblastomas express mesenchymal stem-like properties. Mol Cancer Res 4, 607-619.
Visted, T., Enger, P. O., Lund-Johansen, M., and Bjerkvig, R. (2003). Mechanisms of tumor cell invasion and angiogenesis in the central nervous system. Front Biosci 8, e289-304.
Wurmser, A. E., Nakashima, K., Summers, R. G., Toni, N., D'Amour, K. A., Lie, D. C., and Gage, F. H. (2004). Cell fusion-independent differentiation of neural stem cells to the endothelial lineage. Nature 430, 350-356.
Zhao, X., D, D. A., Lim, W. K., Brahmachary, M., Carro, M. S., Ludwig, T., Cardo, C. C., Guillemot, F., Aldape, K., Califano, A., et al. (2009). The N-Myc-DLL3 Cascade Is Suppressed by the Ubiquitin Ligase Huwel to Inhibit Proliferation and Promote Neurogenesis in the Developing Brain. Dev Cell 17, 210-221.
Zhao, X., Heng, J. I., Guardavaccaro, D., Jiang, R., Pagano, M., Guillemot, F., Iavarone, A., and Lasorella, A. (2008). The HECT-domain ubiquitin ligase Huwel controls neural differentiation and proliferation by destabilizing the N-Myc oncoprotein. Nat Cell Biol 10, 643-653.

### Example 9

### Transient Analysis of Reporters Transfected into Glioma Cells

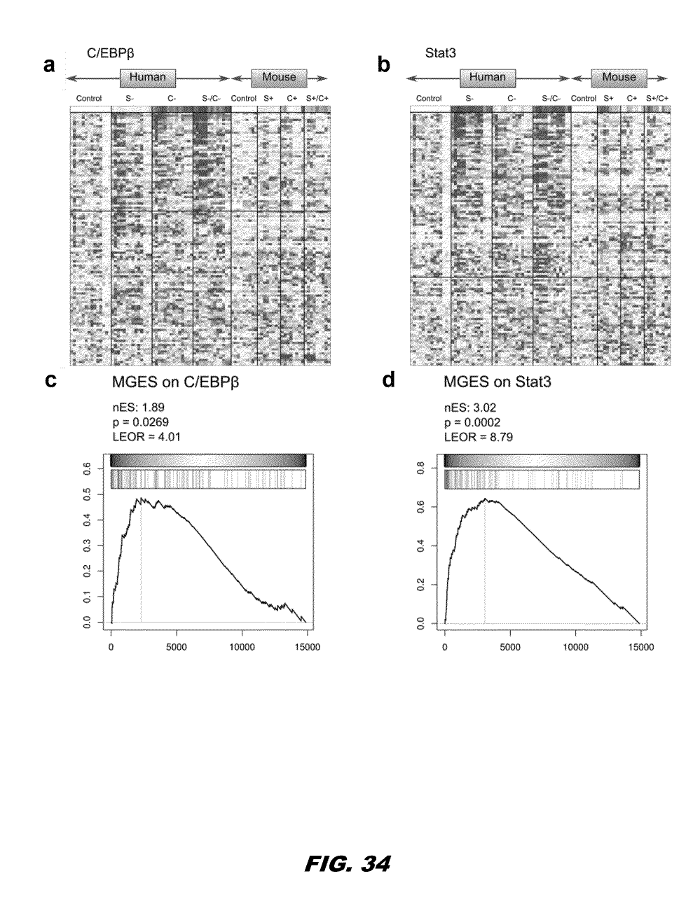

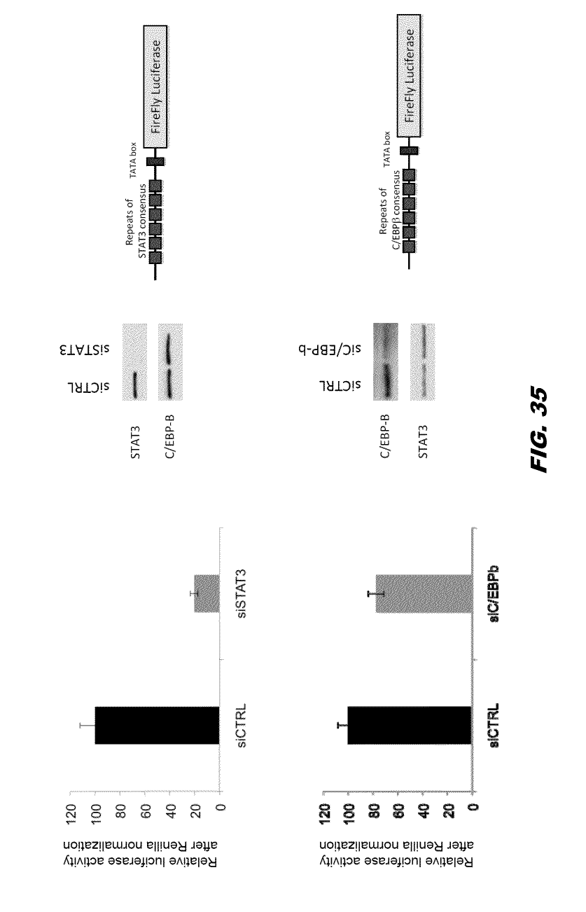

SNB19 human glioma cells were transiently transfected with the plasmids expressing luciferase under the control of the indicated Stat3 or C/EBPbeta binding sites in the presence or absence of siRNA oligonucleotides targeting Stat3 or C/EBPbeta, respectively (FIG. 35). Luciferase activity was measured on a luminometer and the results are shown after normalization with a control renilla-expression vector driven by a CMV-promoter plasmid. Stat3-driven luciferase activity is efficiently down-regulated in cells with silenced Stat3 expression and C/EBPbeta-driven luciferase activity is partially reduced in cells with silenced C/EBPbeta expression.

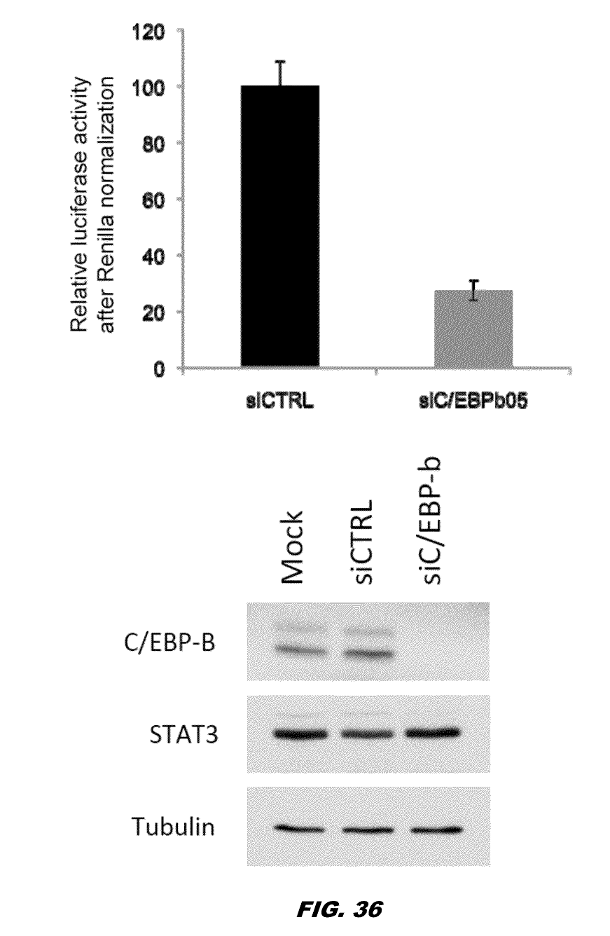

SNB19 human glioma cells were stably transfected with the C/EBPbeta-driven luciferase plasmid (FIG. 36). Several clones were isolated and propagated. Results are shown for clone #9 in combination with cells expressing a control renilla-expression vector driven by a CMV-promoter plasmid (clone #19) (FIG. 36). Cells were transfected with control siRNAs or siRNA oligonucleotides targeting C/EBPbeta (for example, SEQ ID NO: 228 or SEQ ID NO: 229). The control siRNA sequence is the Dharmacon ON-TARGETpIus Non-targeting Pool (Cat#: D-001810-10-20). Luciferase activity was measured on a luminometer and the results are shown after normalization with renilla. C/EBPbeta-driven luciferase activity is efficiently down-regulated in cells with silenced C/EBPbeta expression.

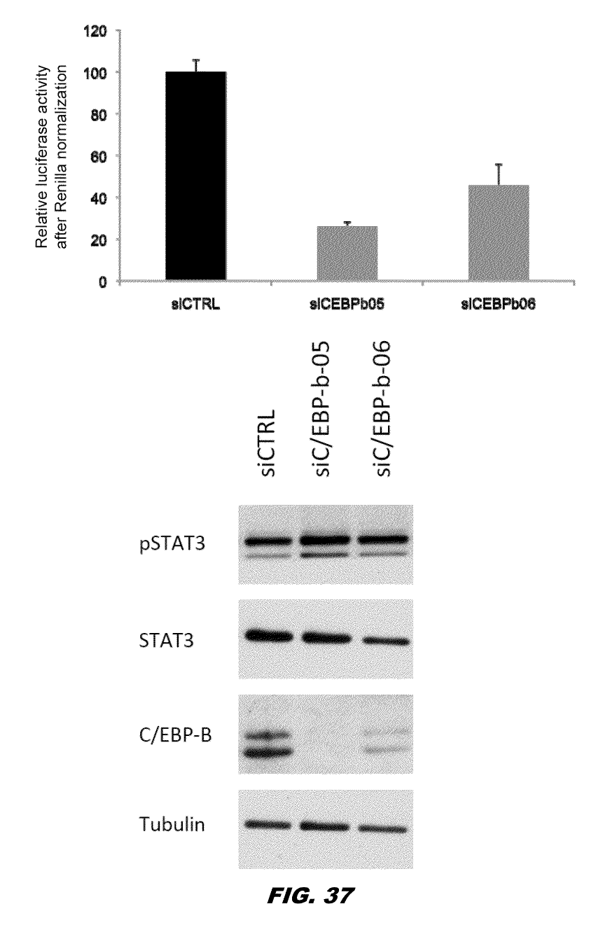

SNB19 human glioma cells were stably transfected with the C/EBPbeta-driven luciferase plasmid (FIG. 37). Several clones were isolated and propagated. Results are shown for clone #9 in combination with cells expressing a control renilla-expression vector driven by a CMV-promoter plasmid (clone #19). Cells were transfected with control siRNAs or two different siRNA oligonucleotides targeting C/EBPbeta (siCEBPb05: CCUCGCAGGUCAAGAGCAA [SEQ ID NO: 228]; and siCEBP06: CUGCUUGGCUGCUGCGUAC [SEQ ID NO: 229]) (FIG. 37). The control siRNA sequence is the Dharmacon ON-TARGETplus Non-targeting Pool (Cat#: D-001810-10-20). Luciferase activity was measured on a luminometer and the results are shown after normalization with renilla. There is a correlation between the efficiency of down-regulation of C/EBPbeta-driven luciferase activity and the efficiency of silencing C/EBPbeta expression.

SNB19 human glioma cells will be stably transfected with the Stat3-driven luciferase plasmid (FIG. 35). Several clones will be isolated and propagated. Cells will then be transfected with control siRNAs or an siRNA oligonucleotide targeting Stat3 (for example, CAGCCUCUCUGCAGAAUUCAA [SEQ ID NO: 230). The control siRNA sequence used will be the Dharmacon ON-TARGETpIus Non-targeting Pool (Cat#: D-001810-10-20). Luciferase activity will be measured on a luminometer and the results will be normalized with renilla.

SNB19 human glioma cells will also be stably transfected with either a C/EBPδ-driven luciferase plasmid, a RunX1-driven luciferase plasmid, a FosL2-driven luciferase plasmid, a bHLH-B2-driven luciferase plasmid, or a ZNF238-driven luciferase plasmid. Several clones will be isolated and propagated. Cells expressing a C/EBPδ-driven luciferase plasmid, a RunX1-driven luciferase plasmid, a FosL2-driven luciferase plasmid, a bHLH-B2-driven luciferase plasmid, or a ZNF238-driven luciferase plasmid will then be transfected with control siRNAs or an siRNA oligonucleotide(s) targeting either C/EBPδ, RunX1, FosL2, bHLH-B2, or ZNF238, respectively. The control siRNA sequence used will be the Dharmacon ON-TARGETpIus Non-targeting Pool (Cat#: D-001810-10-20). Luciferase activity will be measured on a luminometer and the results will be normalized with renilla.

### Example 10

### Identification of Compounds that Interfere with C/EBP-Mediated Transcriptional Activity

A screening for the identification of compounds that could specifically interfere with C/EBP-mediated transcriptional activity was developed in the mesenchymal glioma cell line SNB19. A multimerized C/EBPbeta-luciferase reporter was stably introduced in SNB19 and a screening of ˜9,800 compounds was done to identify positive candidates. In a first pilot screen with 2000 compounds, the chemotherapeutic drug etoposide was the most specific and potent compound identified by screening a microsource library (2000 compounds) and was found to not only inhibit the CCAAT/enhancer-binding protein (CEBP) luciferase reporter, but also the active form of the signal transducer and activator of transcription 3 protein (phospho-STAT3). From the later screen of ˜9,800 compounds, molecules were identified for their ability to inhibit the reporter signal >50%. The list of the molecules is included in Table 18. Further studies are aimed to determine specificity and validate in multiple in vitro and in vivo systems of glioma.

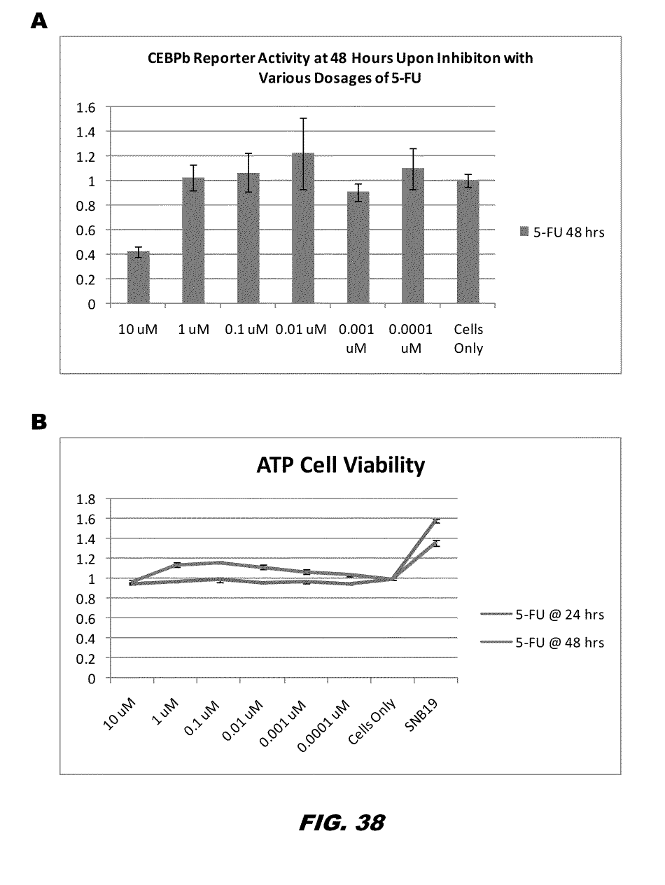

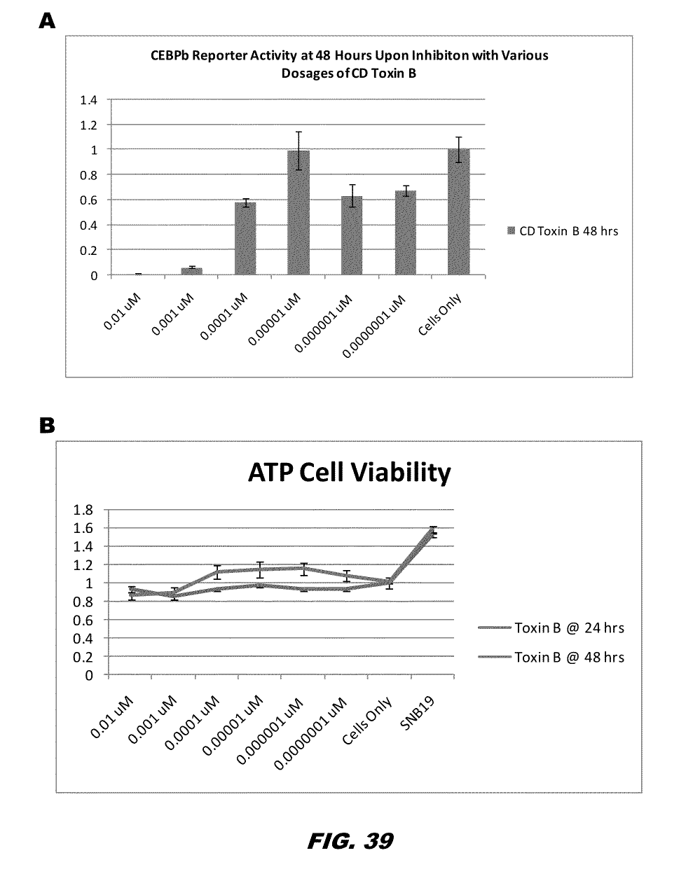

Additional compounds have also been tested for inhibition of C/EBPb activity including 5-fluorouracil and Toxin B from clostridium difticilis. Graphs showing inhibition using a C/EBPb gene reporter assay for both compounds are shown in FIG. 38 and FIG. 39. FIG. 38A shows CEBPb reporter activity at 48 hr upon inhibition with various dosages of 5-fluorouracil (5-FU). FIG. 38B shows ATP cell viability at 24 hr and 48 hr upon inhibition with various dosages of 5-FU. FIG. 39A shows CEBPb reporter activity at 48 hr upon inhibition with various dosages of clostridium difficilis Toxin B (CD Toxin B). FIG. 39B shows ATP cell viability at 24 hr and 48 hr upon inhibition with various dosages of CD Toxin B.

TABLE 18
Compounds that inhibit C/EBPbeta-luciferase reporter signal > 50%.

| ID | Vendor | Structure |
|---|---|---|
| STOCK6S-71833 | IBS | 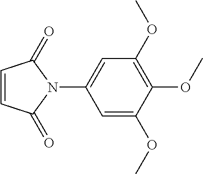 [SMILES: COc1cc(N2C(=O)C=CC2=O)cc(OC)c1OC] |
| BAS 00293383 | Asinex | 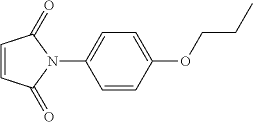 [SMILES: CCCOc1ccc(N2C(=O)C=CC2=O)cc1] |
| BAS 00702176 | Asinex | 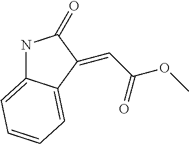 |
| ST057175 | TimTech | 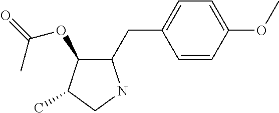 [SMILES: COc1ccc(CC2NC[C@H](C)[C@H]2OC(C)=O)cc1] |
| F3205-0060 | Life | 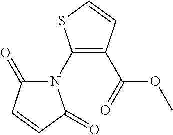 [SMILES: COC(=O)c1ccsc1N1C(=O)C=CC1=O] |
| 9123281 | ChemBridge | 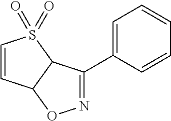 [SMILES: O=S1(=O)C=CC2ON=C(c3ccccc3)C21] |
| BAS 01109087 | Asinex | 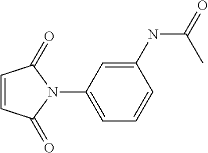 [SMILES: CC(=O)Nc1cccc(N2C(=O)C=CC2=O)c1] |
| T5756459 | Enamine | 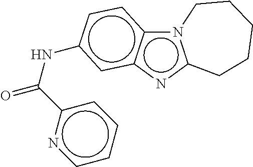 |
| T0505-3249 | Enamine | 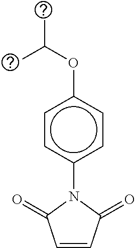 |
| BAS 00389694 | Asinex | 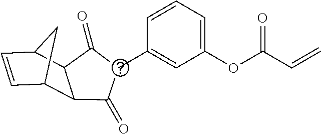 [SMILES: C=CC(=O)Oc1cccc(C2C(=O)C3C4C=CC(C4)C3C2=O)c1] |
| STOCK6S-69648 | IBS | 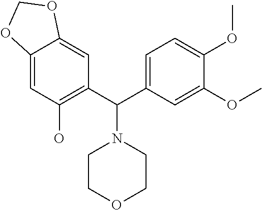 [SMILES: COc1ccc(C(c2cc3c(cc2O)OCO3)N2CCOCC2)cc1OC] |
| STOCK1S-41380 | IBS | 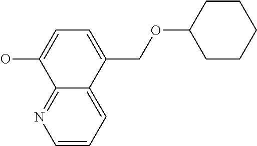 [SMILES: Oc1ccc(COC2CCCCC2)c2cccnc12] |
| F1065-0197 | Life | 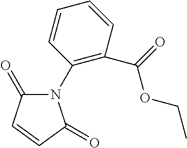 [SMILES: CCOC(=O)c1ccccc1N1C(=O)C=CC1=O] |
| ASN 16287147 | Asinex | 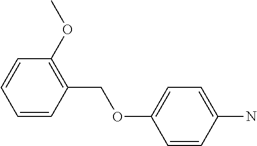 [SMILES: COc1ccccc1COc1ccc(N)cc1] |
| BAS 00873812 | Asinex | 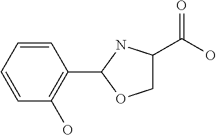 [SMILES: O=C(O)C1COC(c2ccccc2O)N1] |
| T6261669 | Enamine | 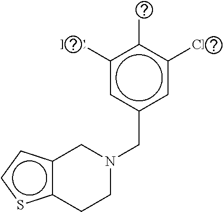 |
| 5338884 | ChemBridge | 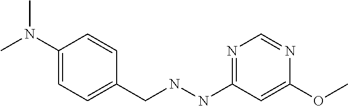 |
| STOCK2S-10951 | IBS | 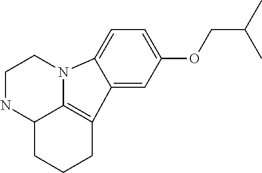 [SMILES: CC(C)COc1ccc2c(c1)c1c3n2CCNC3CCC1] |
| STOCK6S-65265 | IBS | 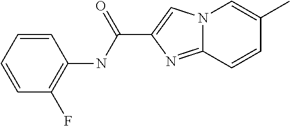 [SMILES: Cc1ccc2nc(C(=O)Nc3ccccc3F)cn2c1] |
| STOCK1S-62600 | IBS | 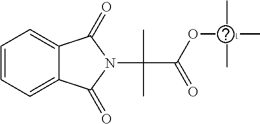 [SMILES: CC(C)(C(=O)[O][Sn]([CH3])([CH3])[CH3])N1C(=O)c2ccccc2C1=O] |
| BAS 02946522 | Asinex | 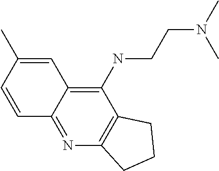 [SMILES: Cc1ccc2nc3c(c(NCCN(C)C)c2c1)CCC3] |
| BAS 00318863 | Asinex | 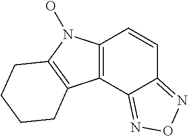 [SMILES: On1c2c(c3c4nonc4ccc31)CCCC2] |
| T5225535 | Enamine | |
| BAS 02256215 | Asinex | 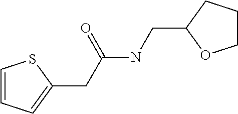 [SMILES: O=C(Cc1cccs1)NCC1CCCO1] |
| T5756959 | Enamine | 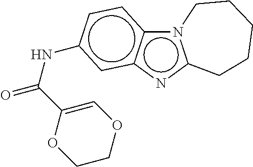 |
| 5106399 | ChemBridge | 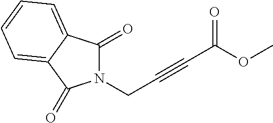 [SMILES: COC(=O)C#CCN1C(=O)c2ccccc2C1=O] |
| ST057180 | TimTech | 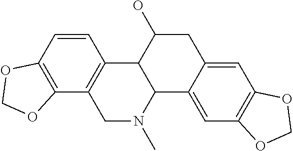 [SMILES: CN1Cc2c(ccc3c2OCO3)C2C(O)Cc3cc4c(cc3C21)OCO4] |
| STOCK2S-14814 | IBS | 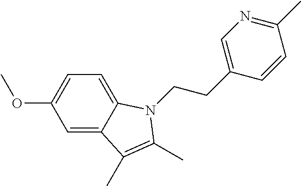 |
| T0519-1108 | Enamine | 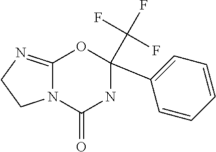 [SMILES: O=C1NC(c2ccccc2)(C(F)(F)F)OC2=NCCN12] |
| T5552290 | Enamine | 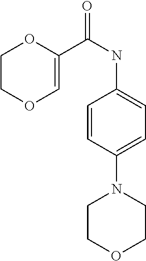 [SMILES: O=C(Nc1ccc(N2CCOCC2)cc1)C1=COCCO1] |
| ST084242 | TimTech | 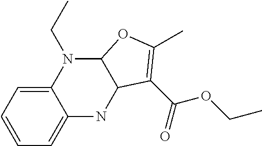 [SMILES: CCOC(=O)C1=C(C)OC2C1Nc1ccccc1N2CC] |
| STOCK4S-73514 | IBS | 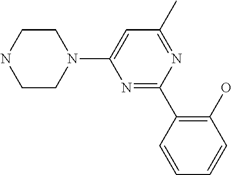 [SMILES: Cc1cc(N2CCNCC2)nc(-c2ccccc2O)n1] |
| BAS 02140954 | Asinex | 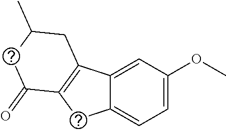 [SMILES: COc1ccc2[nH]c3c(c2c1)CC(C)NC3=O] |
| STOCK6S-76426 | IBS | 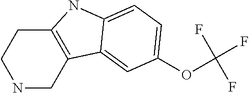 [SMILES: FC(F)(F)Oc1ccc2[nH]c3c(c2c1)CNCC3] |
| BAS 02592298 | Asinex | 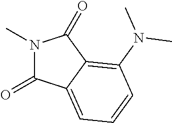 |
| 5570087 | ChemBridge | 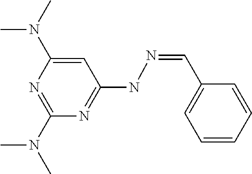 [SMILES: CN(C)c1cc(N/N=C\c2ccccc2)nc(N(C)C)n1] |
| T5636062 | Enamine | 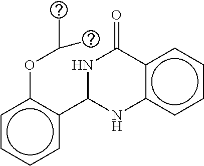 |
| ASN 07731410 | Asinex | 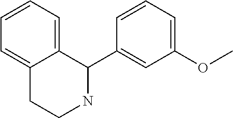 [SMILES: COc1cccc(C2NCCc3ccccc32)c1] |
| T5414273 | Enamine | 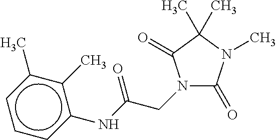 |
| BAS 01236576 | Asinex | 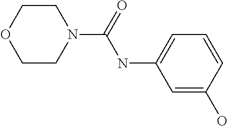 [SMILES: O=C(Nc1cccc(O)c1)N1CCOCC1] |
| T5691624 | Enamine | 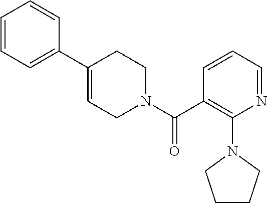 [SMILES: O=C(c1cccnc1N1CCCC1)N1CC=C(c2ccccc2)CC1] |
| STOCK6S-83108 | IBS | 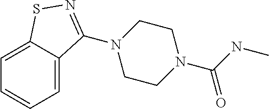 [SMILES: CNC(=O)N1CCN(c2nsc3ccccc23)CC1] |
| ST079841 | TimTech | 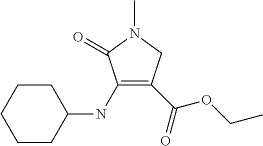 [SMILES: CCOC(=O)C1=C(NC2CCCCC2)C(=O)N(C)C1] |
| STOCK3S-62210 | IBS | 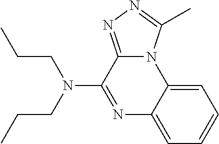 [SMILES: CCCN(CCC)c1nc2ccccc2n2c(C)nnc12] |
| 6102842 | ChemBridge | 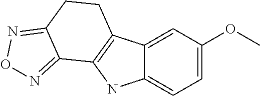 [SMILES: COc1ccc2[nH]c3c(c2c1)CCc1nonc1-3] |
| STOCK2S-03855 | IBS | 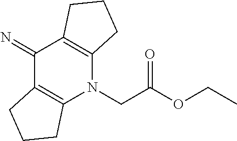 [SMILES: CCOC(=O)Cn1c2c(c(=N)c3c1CCC3)CCC2] |
| ASN 19852408 | Asinex | 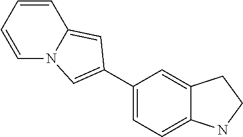 [SMILES: c1ccn2cc(-c3ccc4c(c3)CCN4)cc2c1] |
| T5644376 | Enamine | 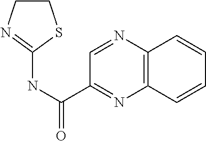 [SMILES: O=C(NC1=NCCS1)c1cnc2ccccc2n1] |
| STOCK3S-68420 | IBS | 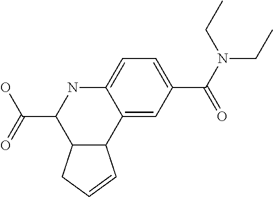 [SMILES: CCN(CC)C(=O)c1ccc2c(c1)C1C=CCC1C(C(=O)O)N2] |
| BAS 06632970 | Asinex | 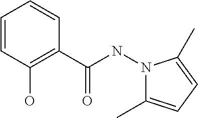 [SMILES: Cc1ccc(C)n1NC(=O)c1ccccc1O] |
| ASN 17325346 | Asinex | 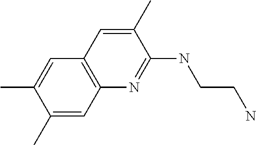 [SMILES: Cc1cc2cc(C)c(NCCN)nc2cc1C] |
| STOCK4S-01916 | IBS | 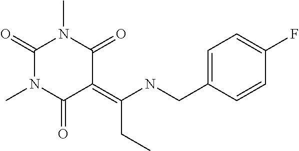 [SMILES: CCC(NCc1ccc(F)cc1)=C1C(=O)N(C)C(=O)N(C)C1=O] |
| ST4093613 | TimTech | 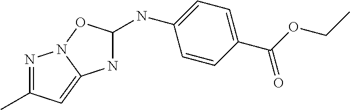 [SMILES: CCOC(=O)c1ccc(NC2Nc3cc(C)nn3O2)cc1] |

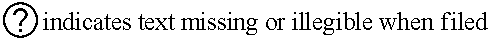

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways to obtain additional embodiments within the scope and spirit of the invention.

What is claimed is:

1. A method for treating nervous system cancer in a subject in need thereof comprising administering to the subject a compound that inhibits a MGES protein.

2. The method of claim 1, wherein the compound is selected from the group consisting of etoposide, 5-fluorouracil, Clostridium difficile Toxin B, 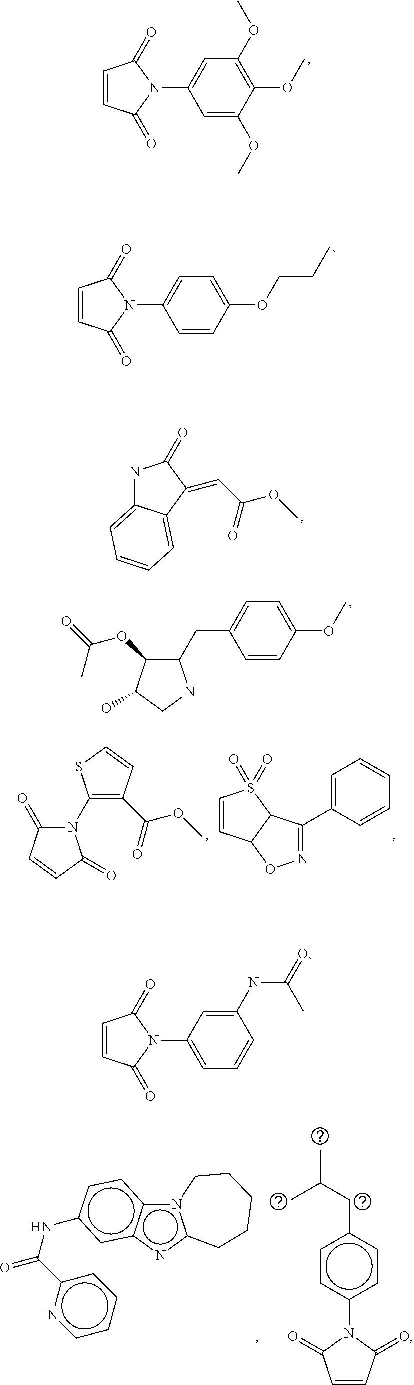 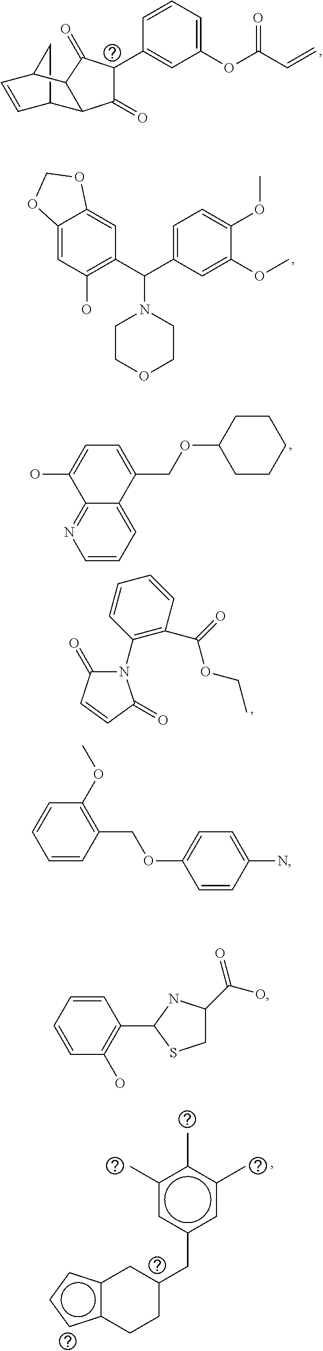 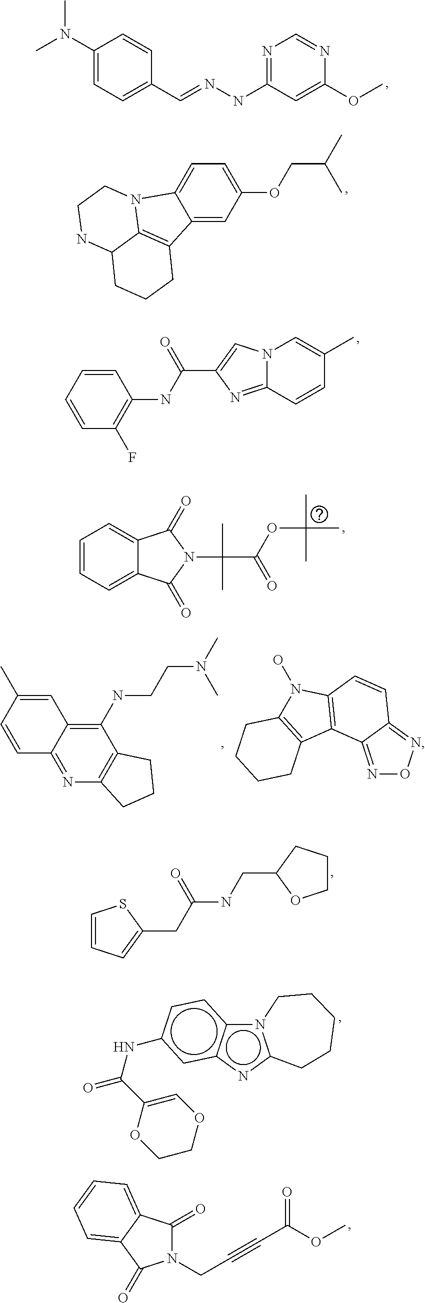 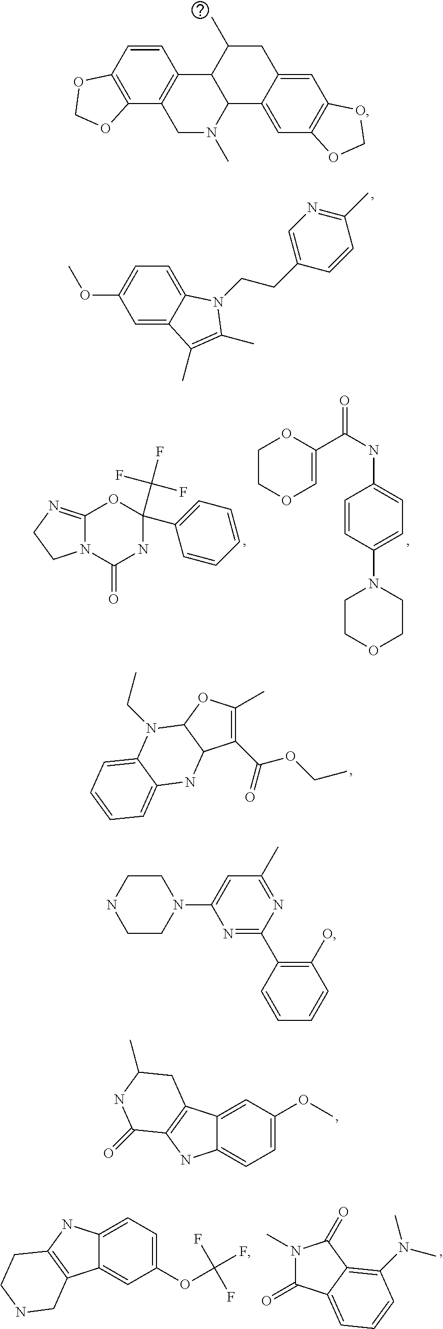 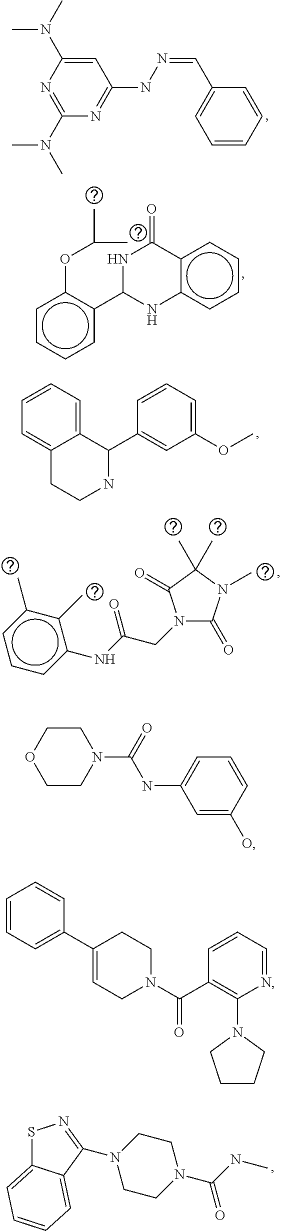 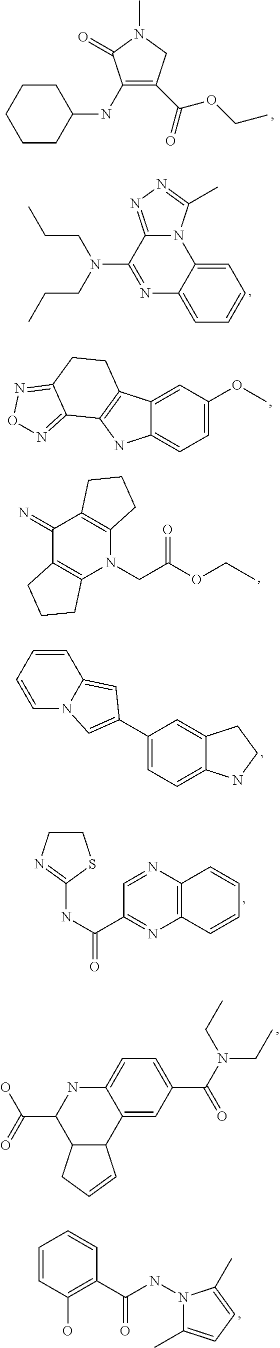 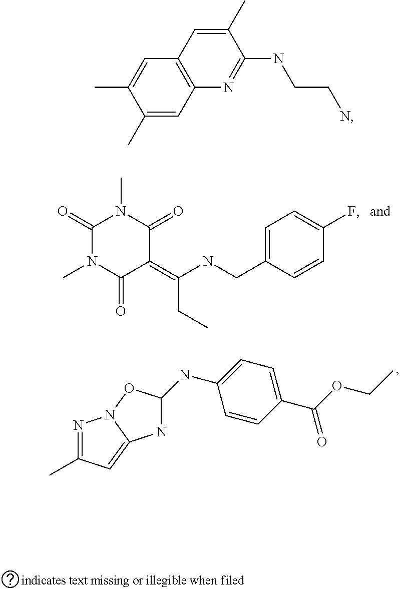
and pharmaceutically acceptable salts thereof.

3. The method of claim 2, wherein the compound is selected from the group consisting of 5-fluorouracil, Clostridium difficile Toxin B, 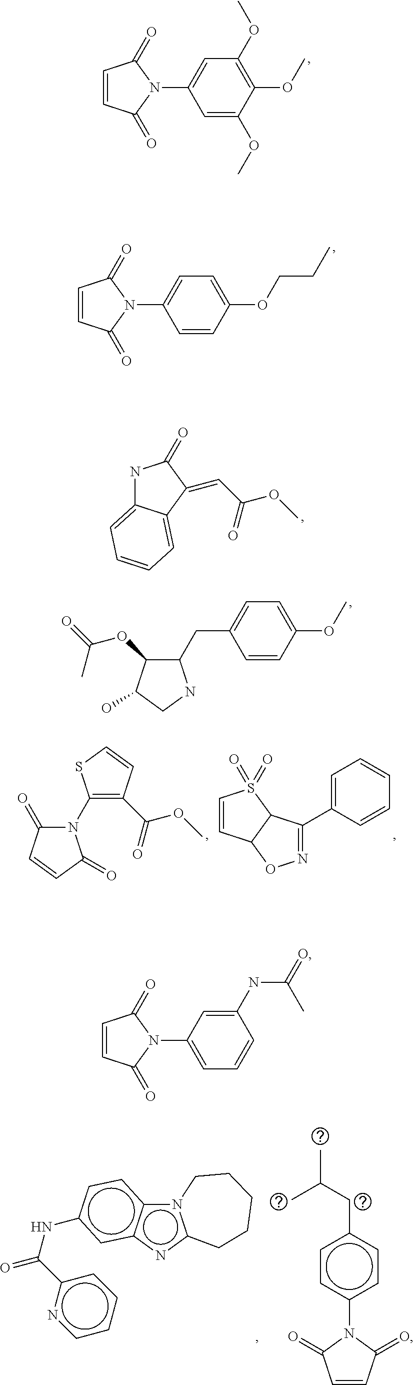 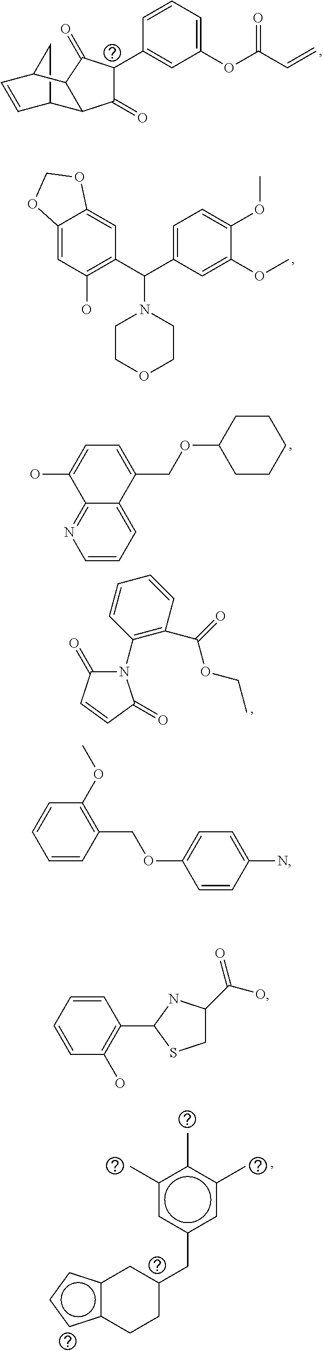 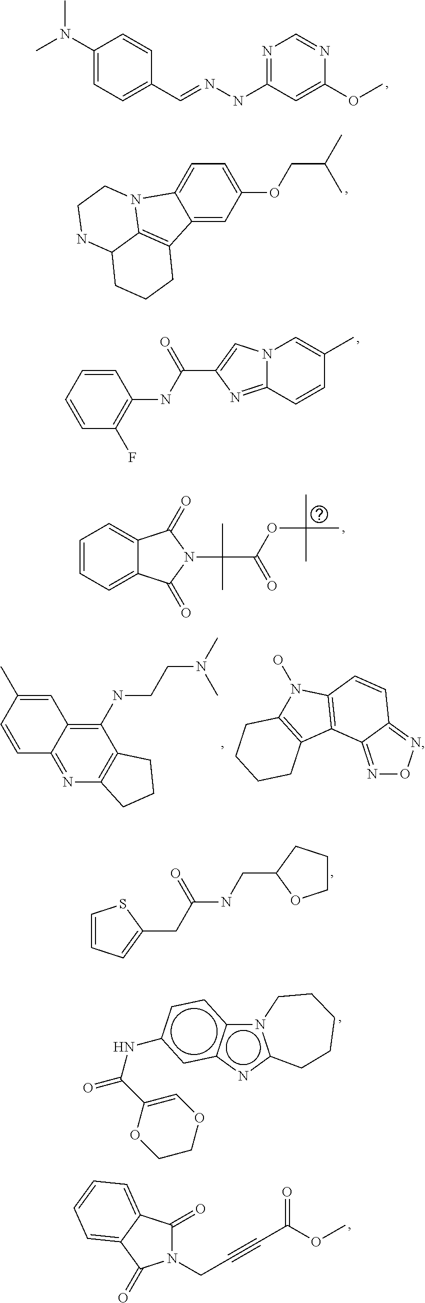 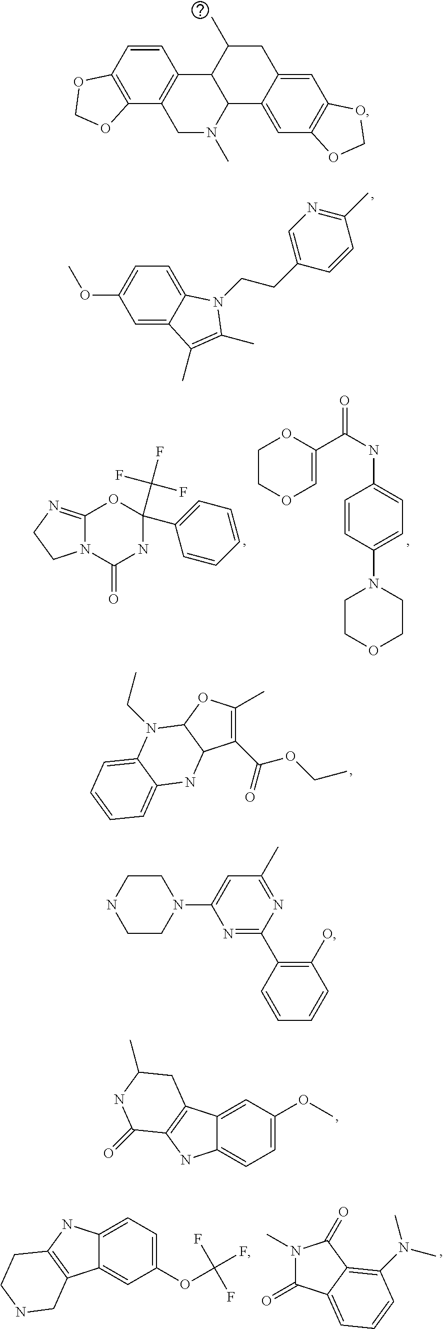 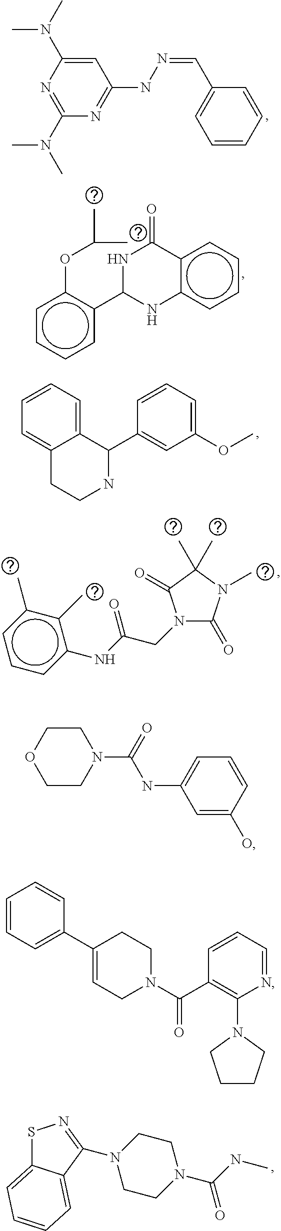 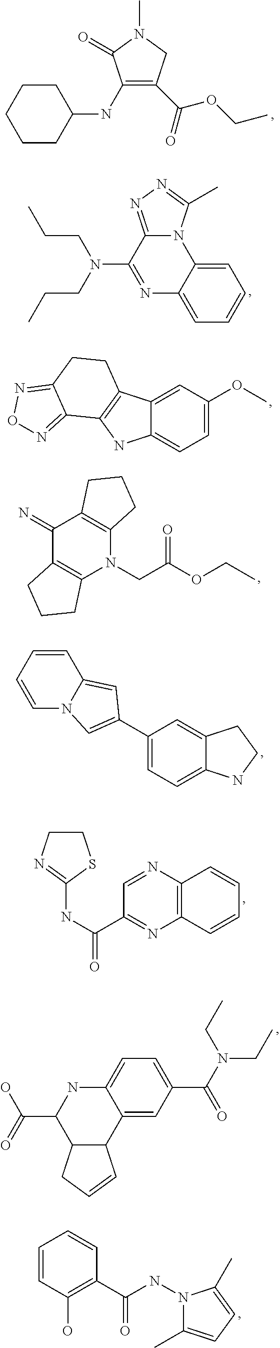 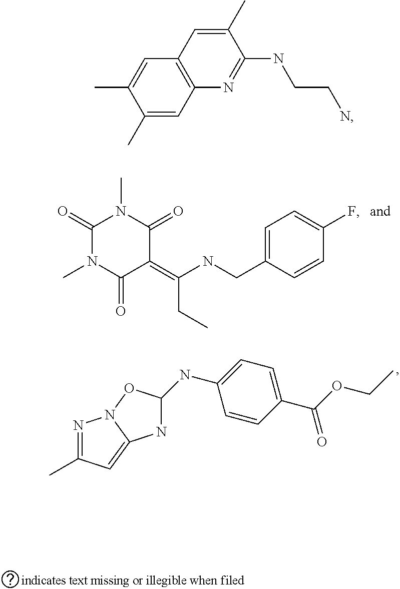
and pharmaceutically acceptable salts thereof.

4. The method of claim 3, wherein the compound is selected from the group consisting of Clostridium difficile Toxin B, 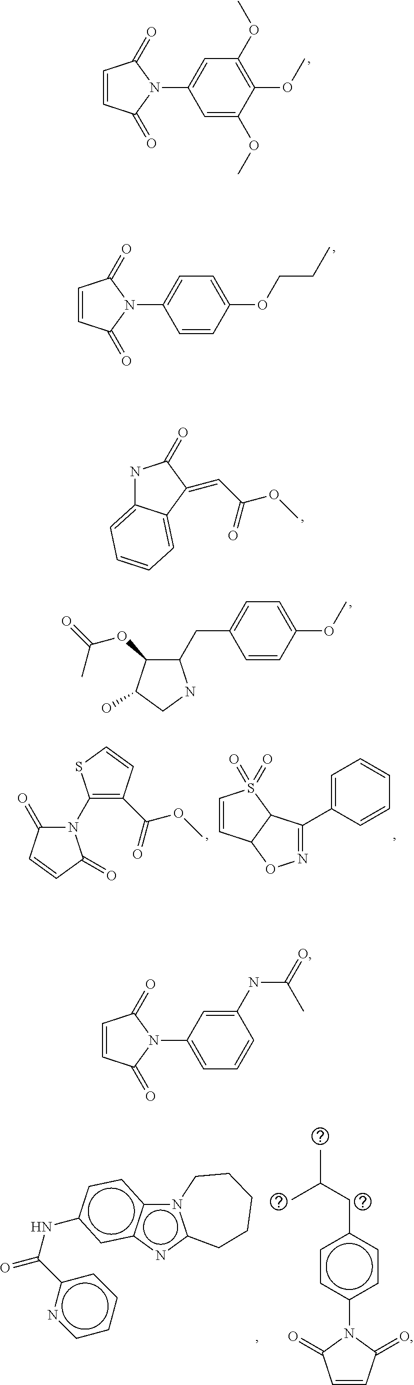 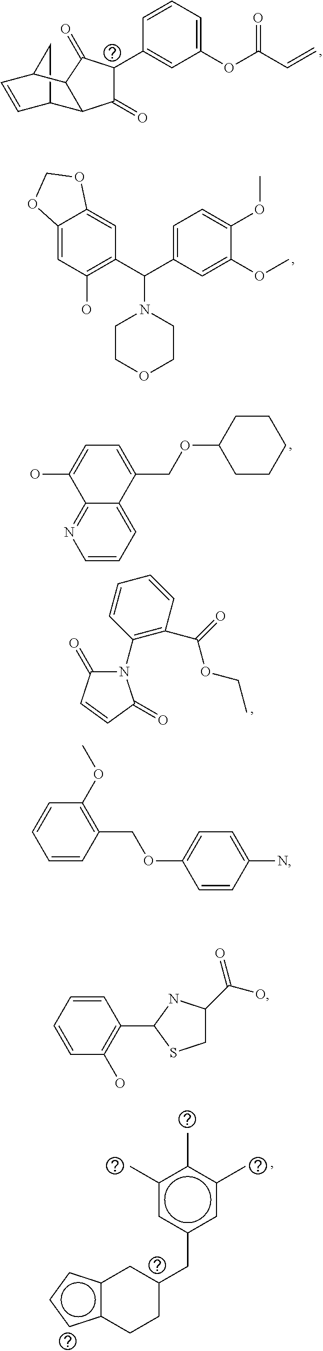 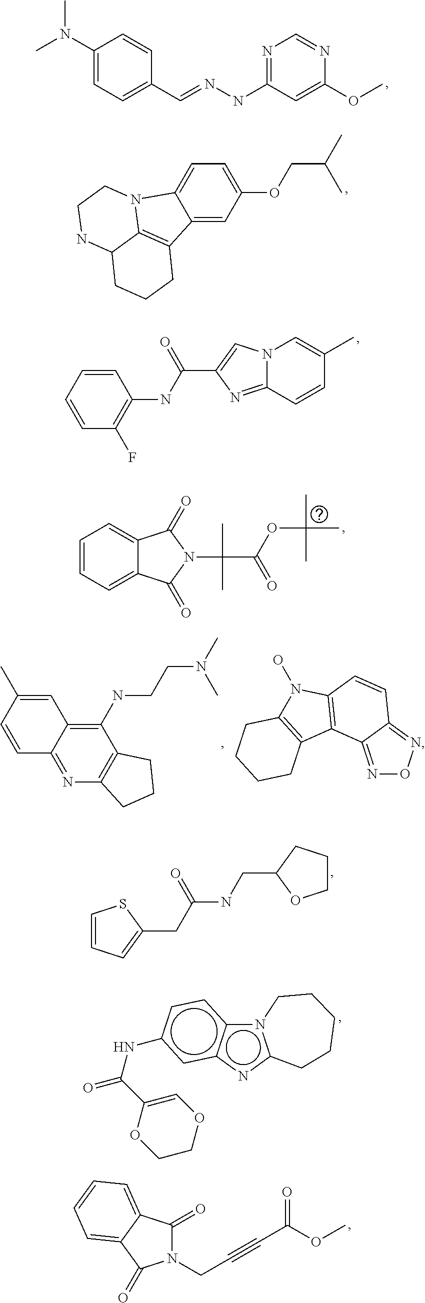 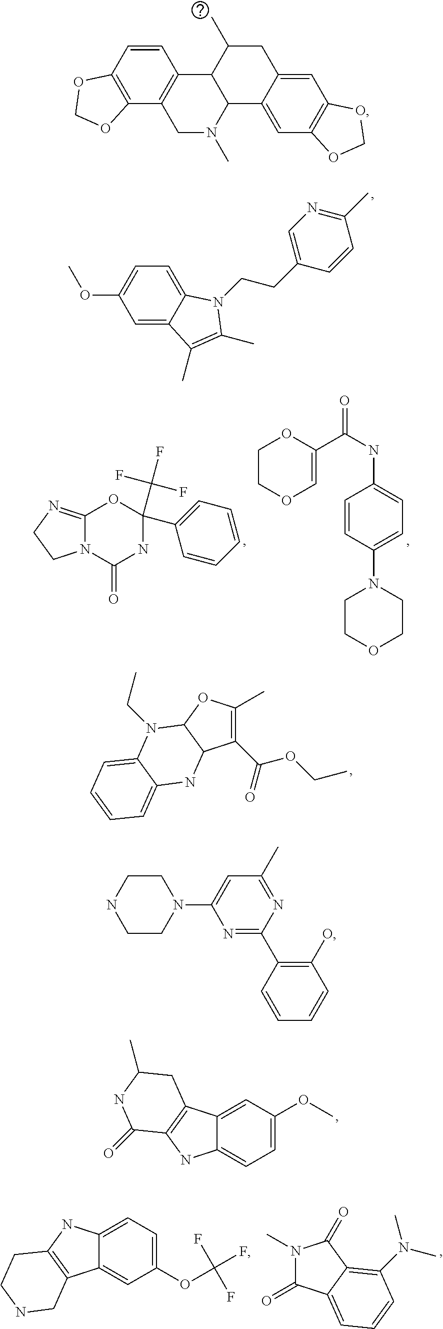 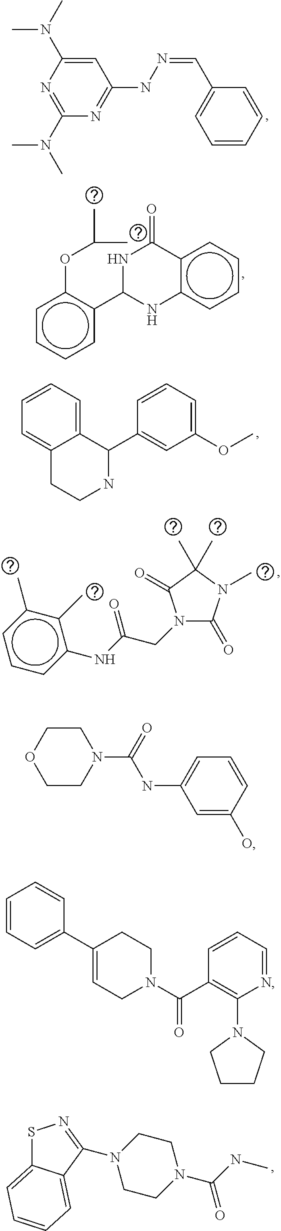 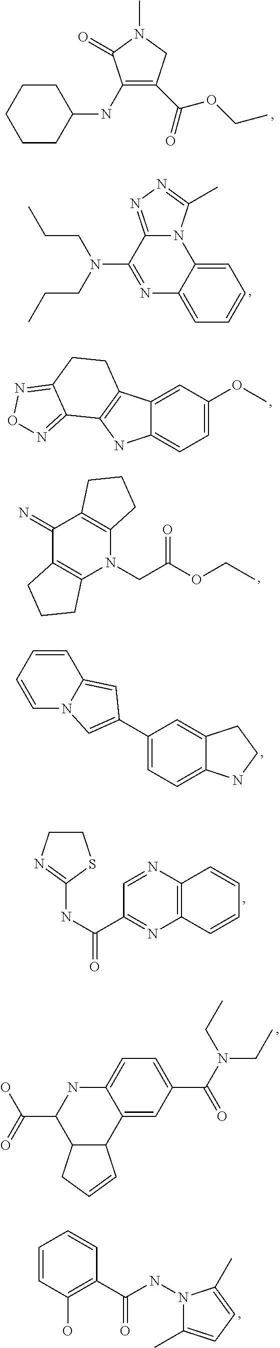 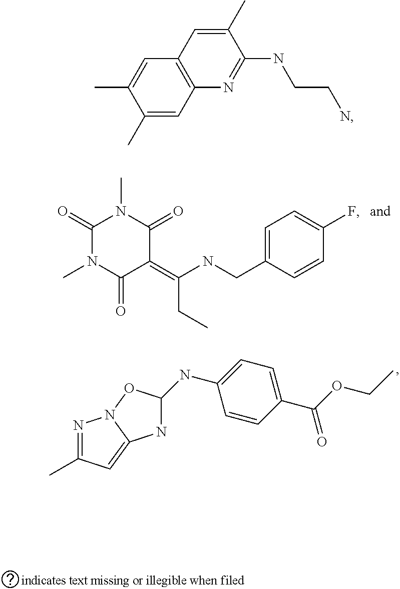
and pharmaceutically acceptable salts thereof.

5. The method of claim 1, wherein the MGES protein is C/EPB or Stat3.

6. The method of claim 1, wherein the cancer is glioma or meningioma.

7. The method of claim 1, wherein the cancer is astrocytoma, Glioblastoma Multiforme, oligodentroglioma, ependymoma or meningioma.

8. The method of claim 1, wherein the cancer is cerebellar astrocytoma, medulloblastoma, ependymona, brain stem glioma, optic nerve glioma, acoustic neuromas, nerve sheath tumors, or germinoma.

9. A method for decreasing MGES protein activity in a subject having a nervous system cancer, the method comprising administering to the subject a compound that inhibits a MGES protein.

10. The method of claim 9, wherein the compound is selected from the group consisting of etoposide, 5-fluorouracil, Clostridium difficile Toxin B, 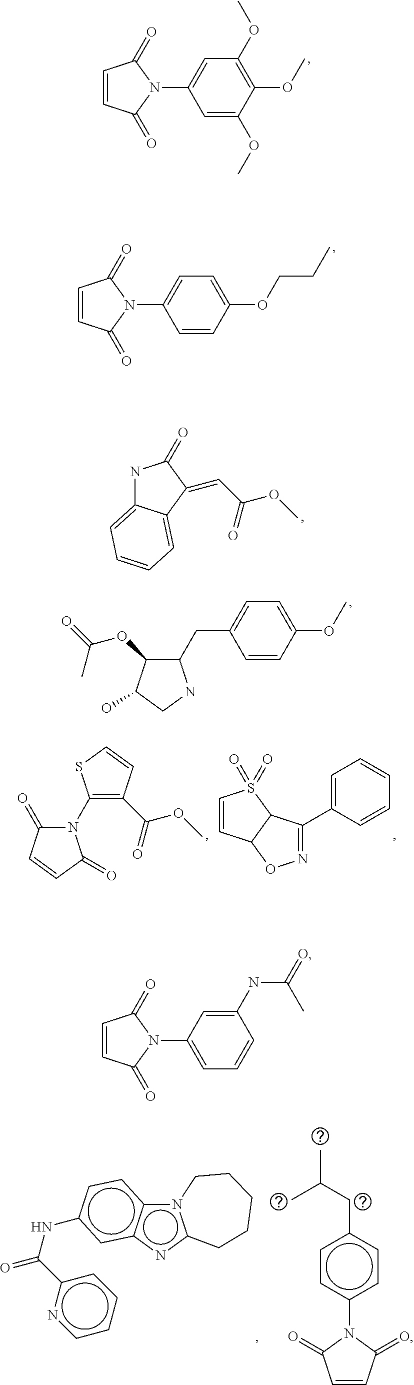 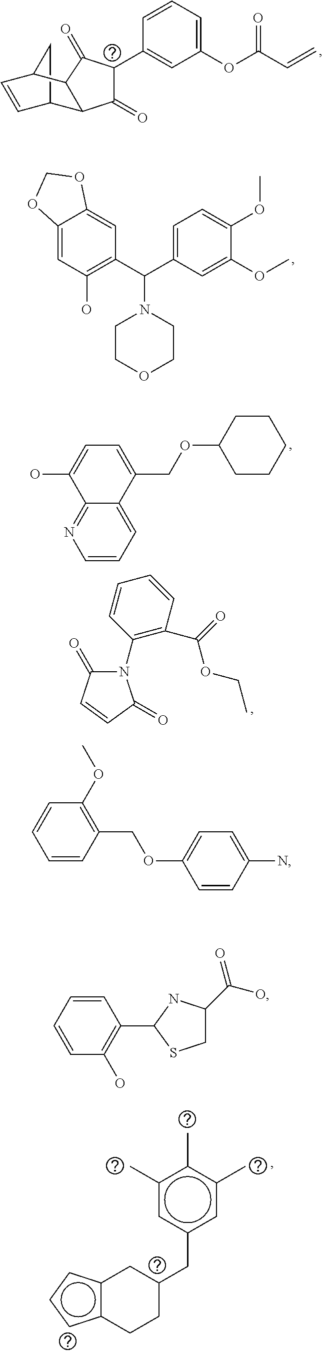 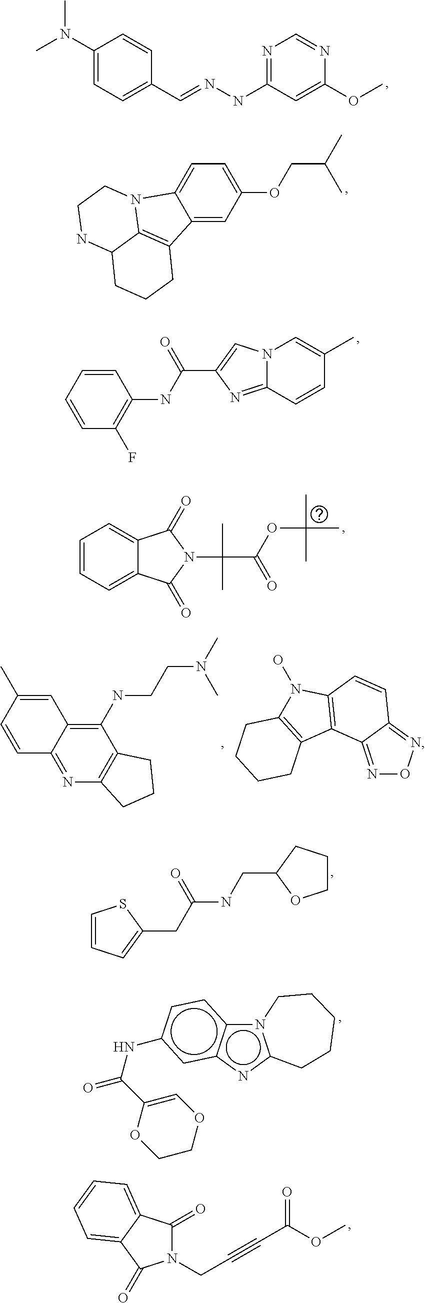 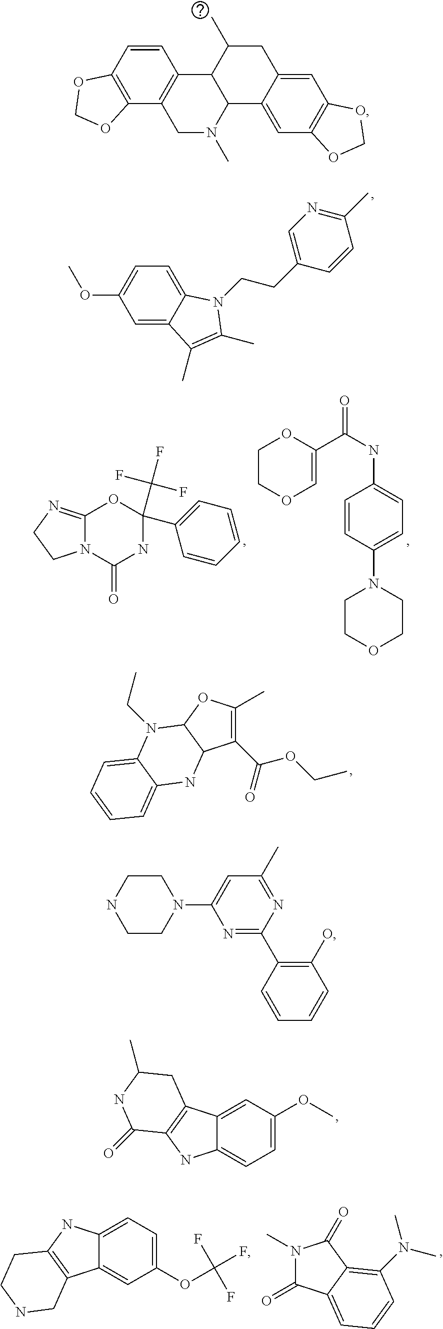 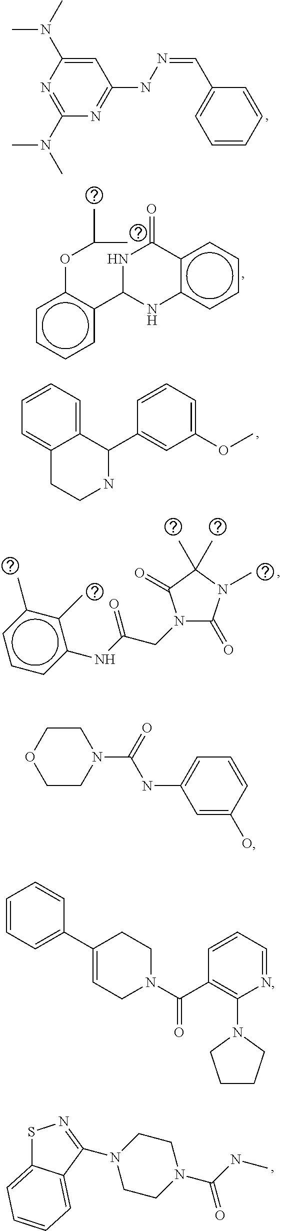 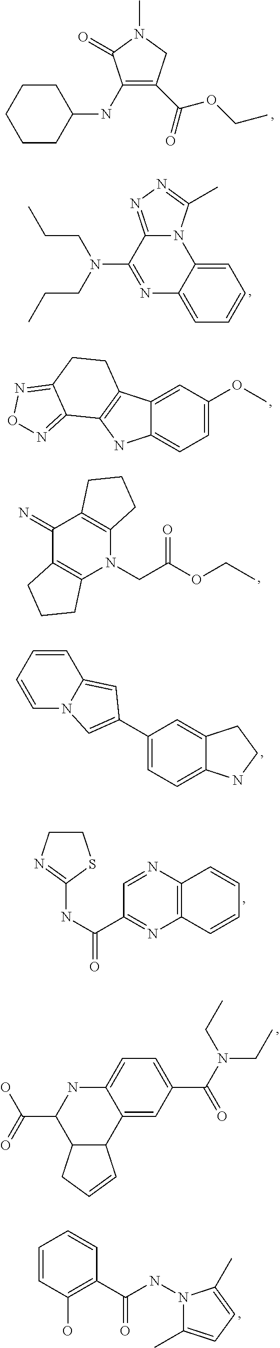 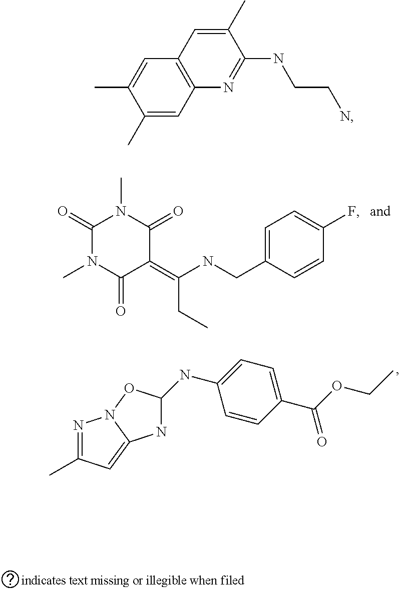
and pharmaceutically acceptable salts thereof.

11. The method of claim 10, wherein the compound is selected from the group consisting of 5-fluorouracil, Clostridium difficile Toxin B, 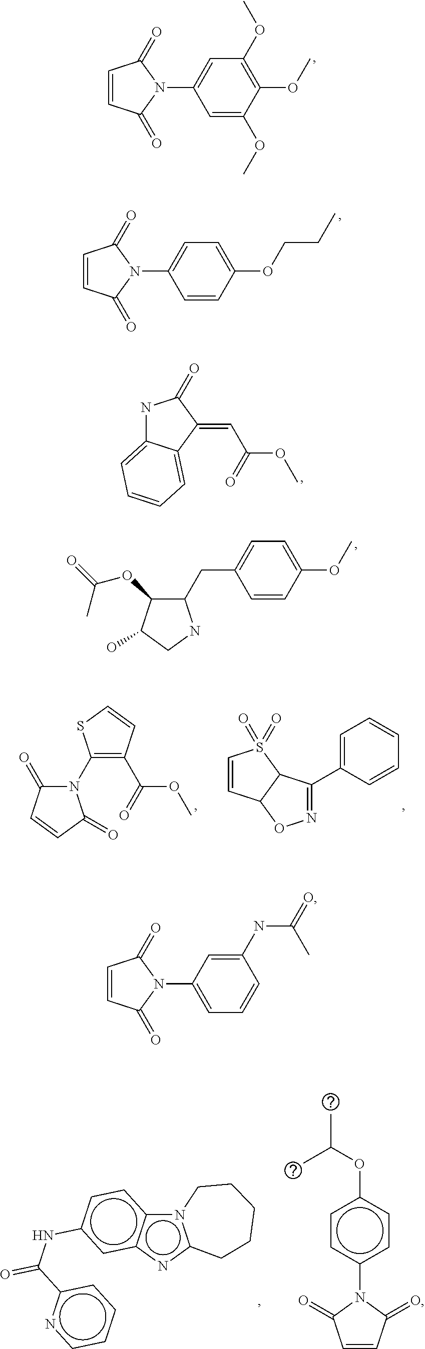 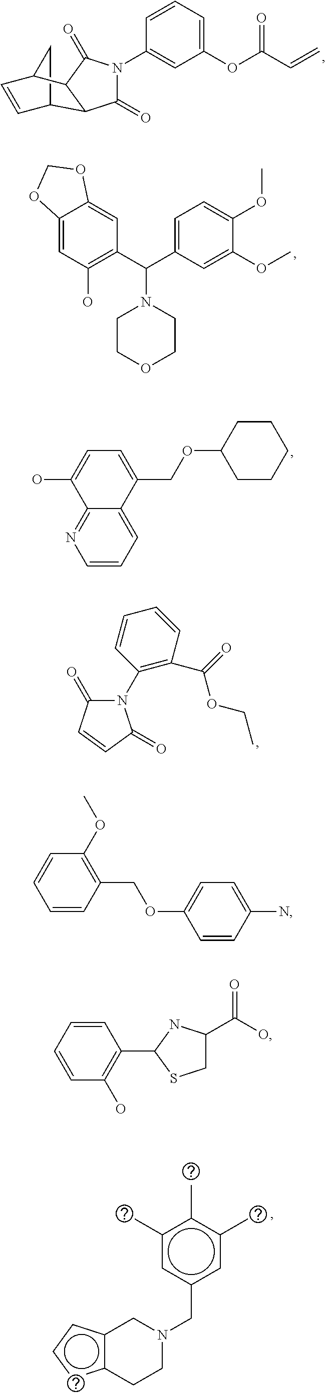 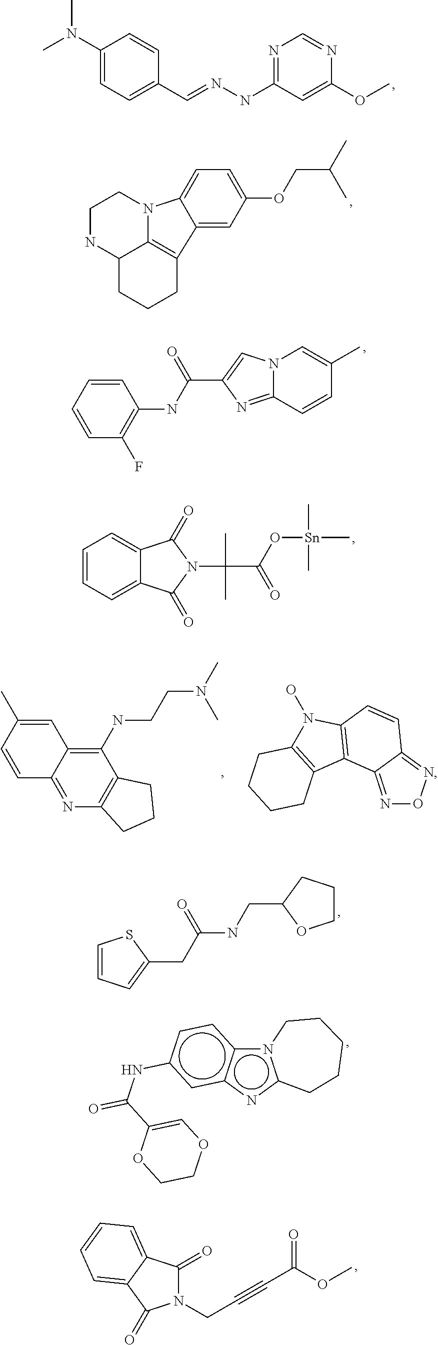 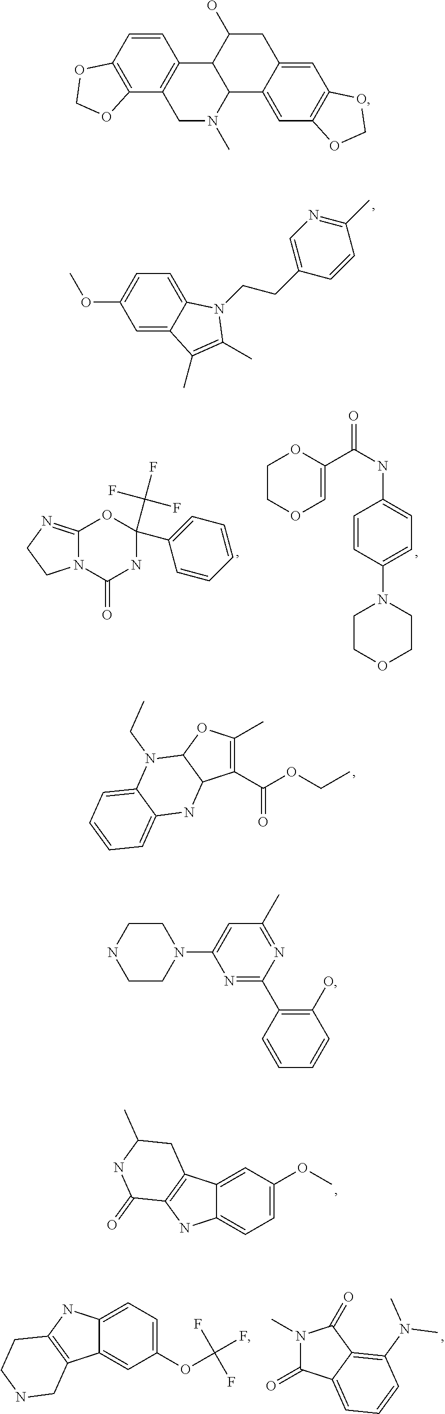 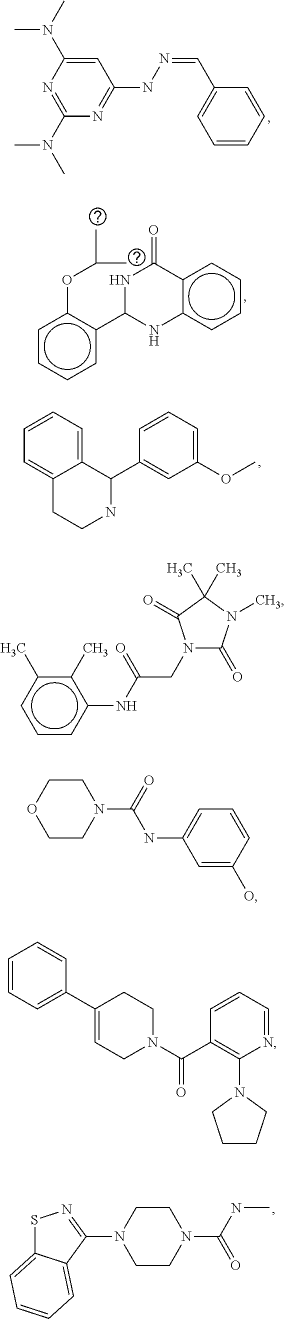 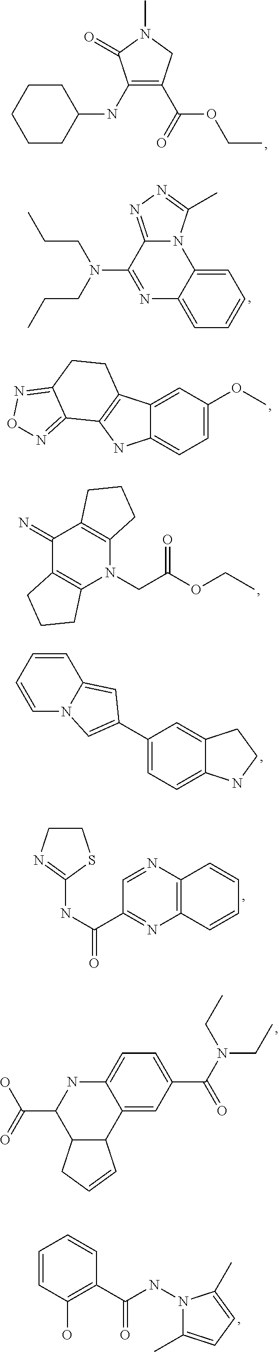 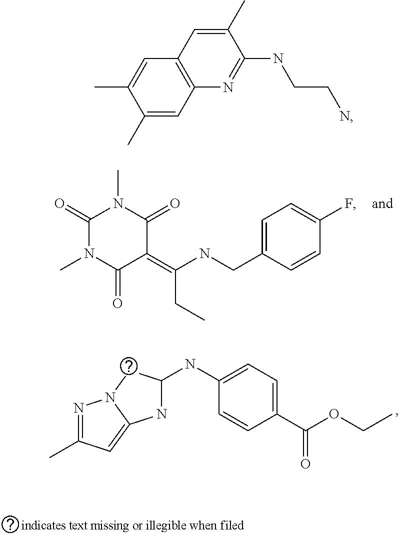
and pharmaceutically acceptable salts thereof.

12. The method of claim 11, wherein the compound is selected from the group consisting of Clostridium difficile Toxin B, 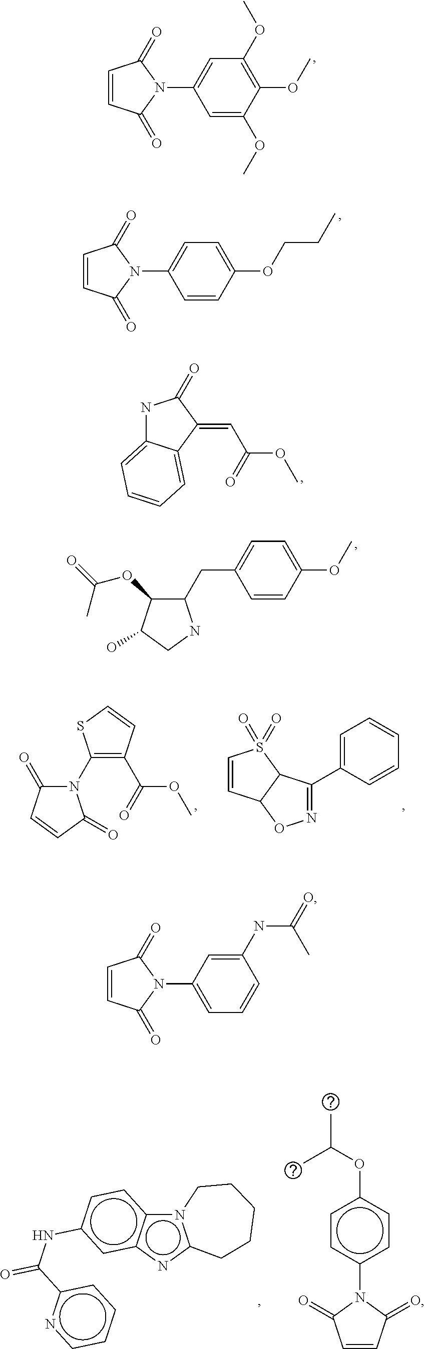 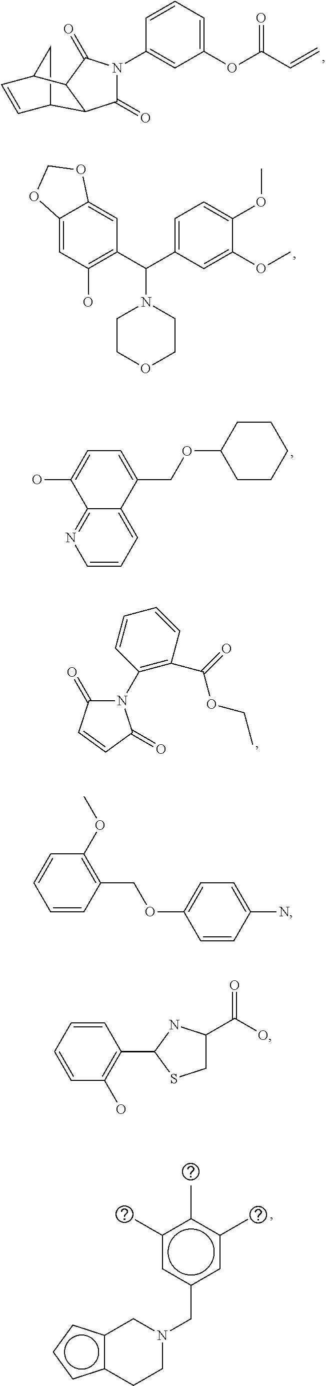 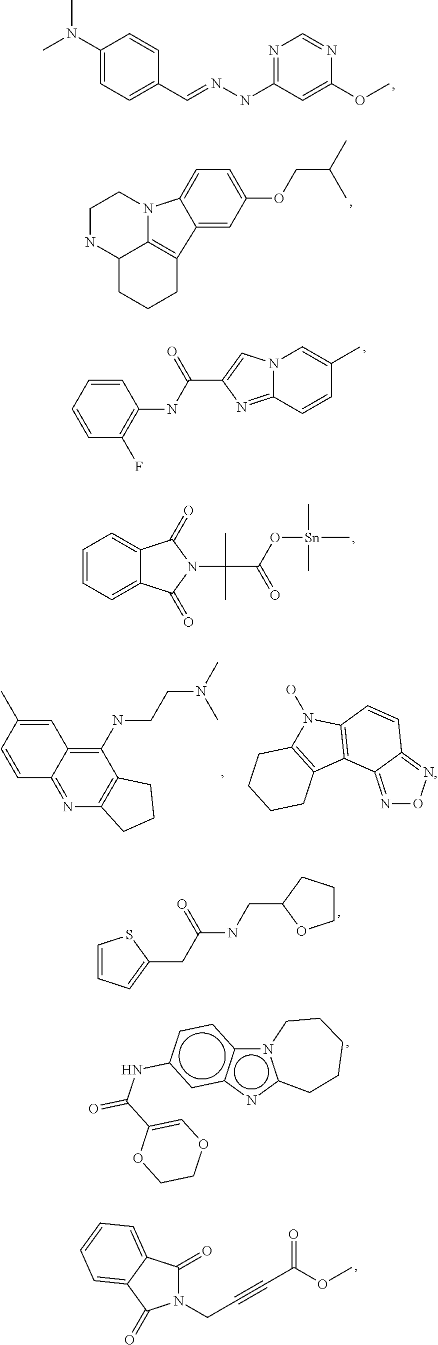 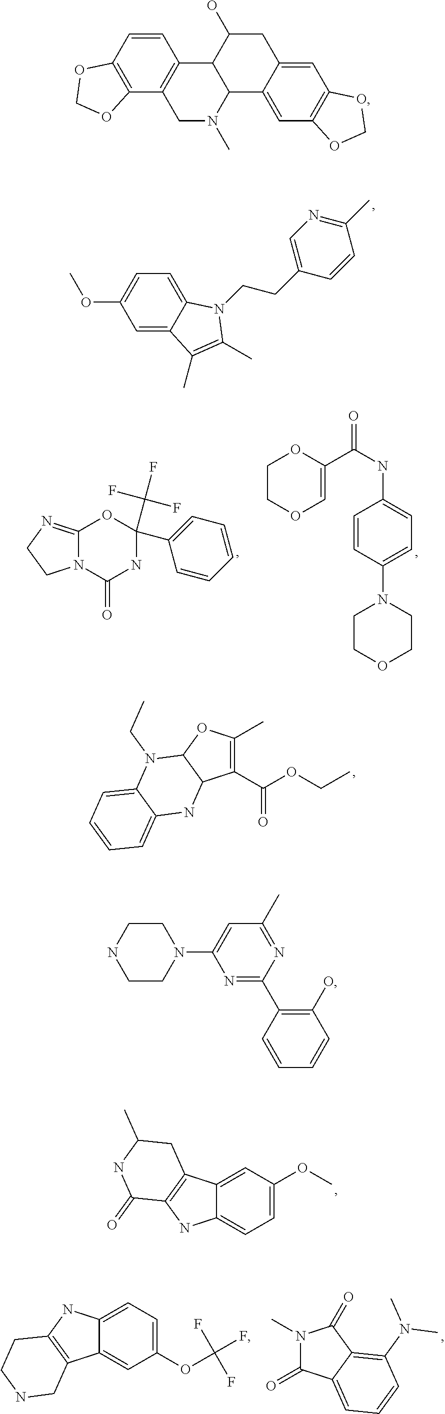 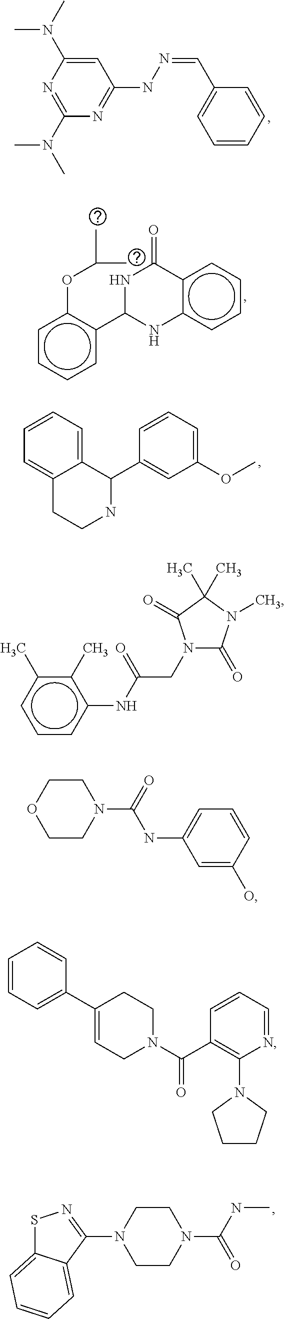 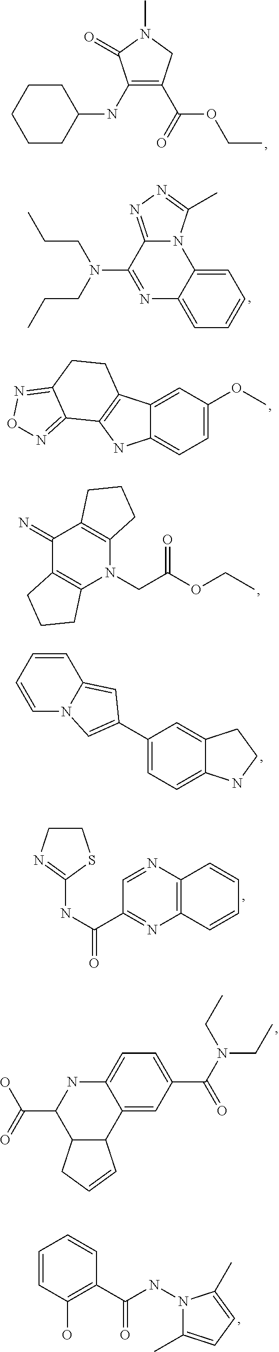 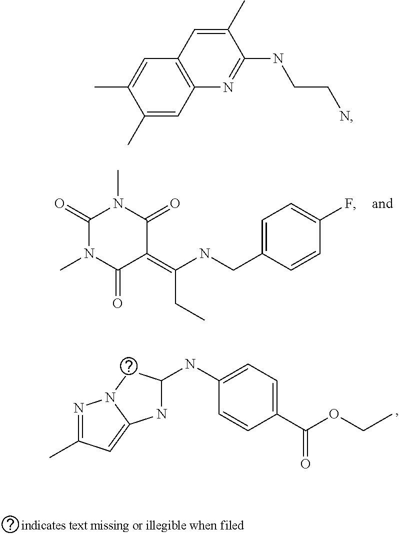
and pharmaceutically acceptable salts thereof.

13. The method of claim 9, wherein the MGES protein is C/EPB or Stat3.

14. The method of claim 9, wherein the cancer is glioma or meningioma.

15. The method of claim 9, wherein the cancer is astrocytoma, Glioblastoma Multiforme, oligodentroglioma, ependymoma or meningioma.

16. The method of claim 9, wherein the cancer is cerebellar astrocytoma, medulloblastoma, ependymona, brain stem glioma, optic nerve glioma, acoustic neuromas, nerve sheath tumors, or germinoma.

17. A method for inhibiting a MGES protein comprising contacting said protein with an effective amount of a compound selected from the group consisting of etoposide, 5-fluorouracil, Clostridium difficile Toxin B, 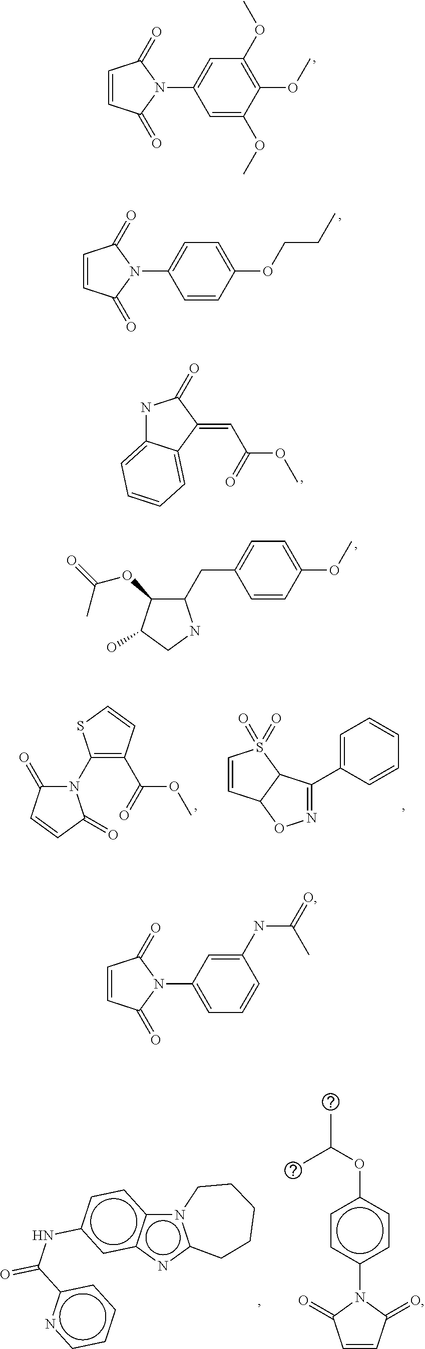 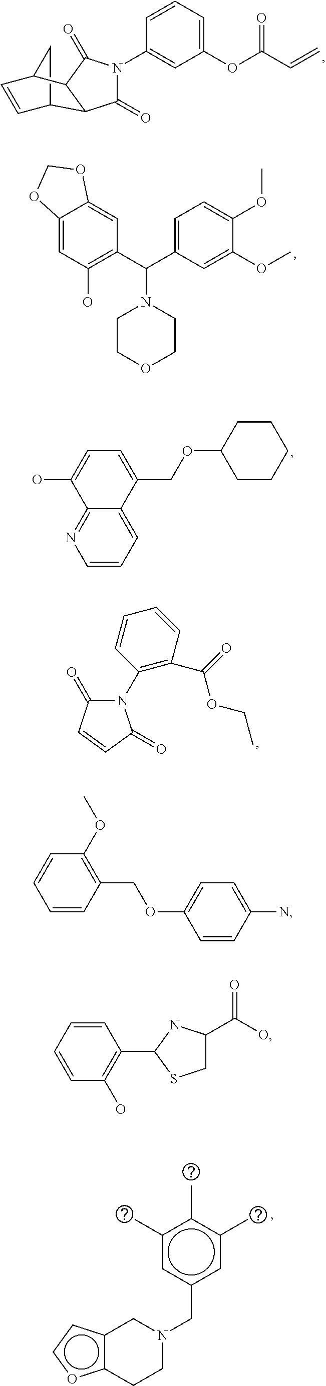 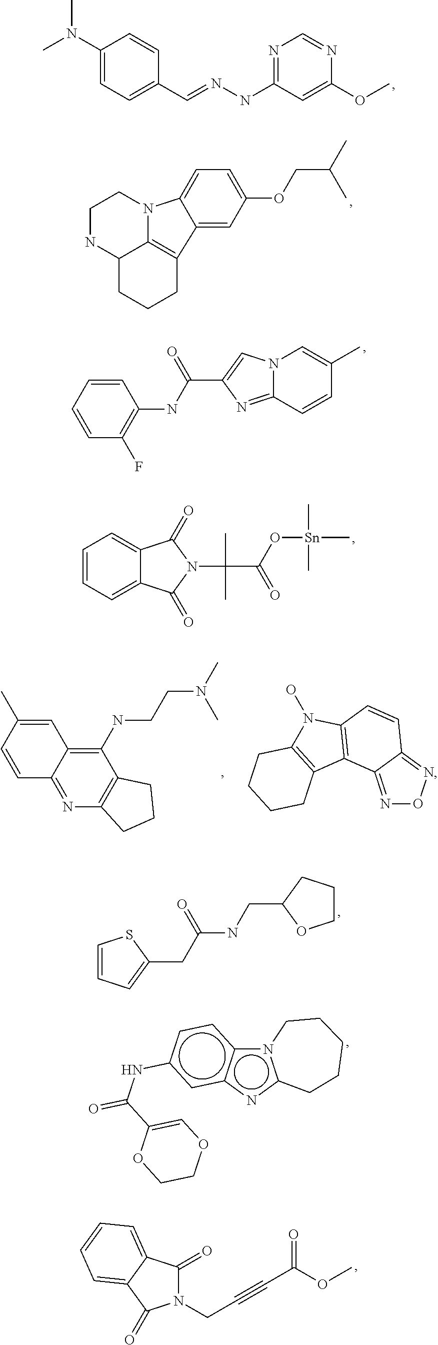 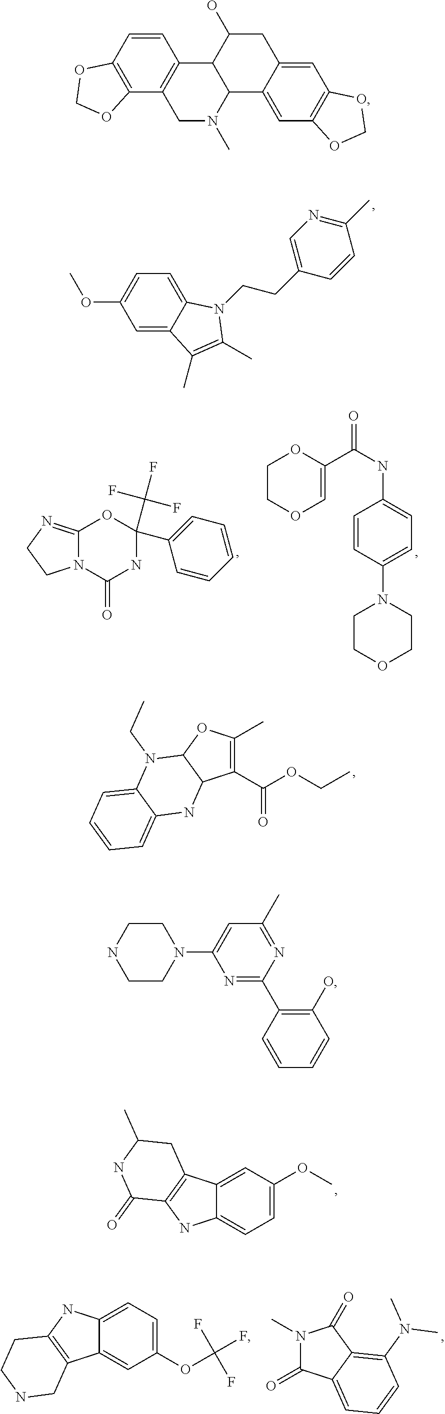 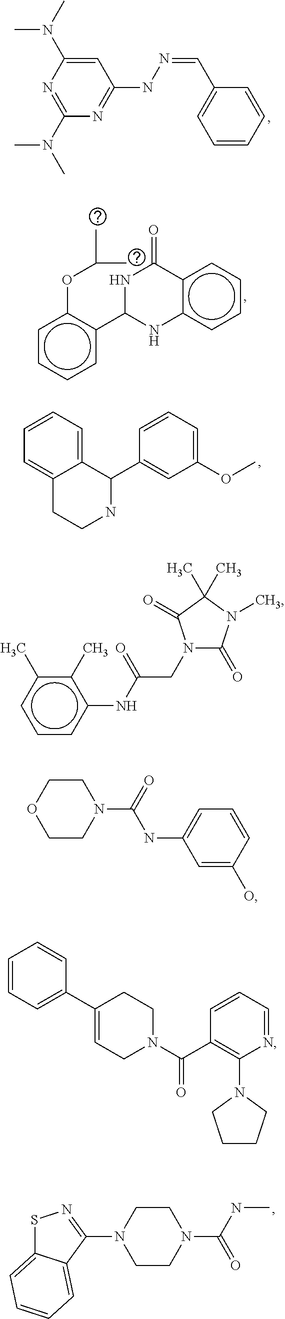 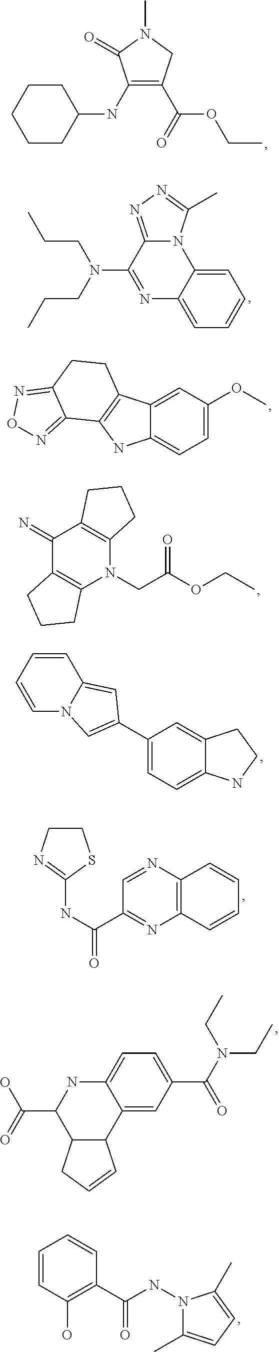 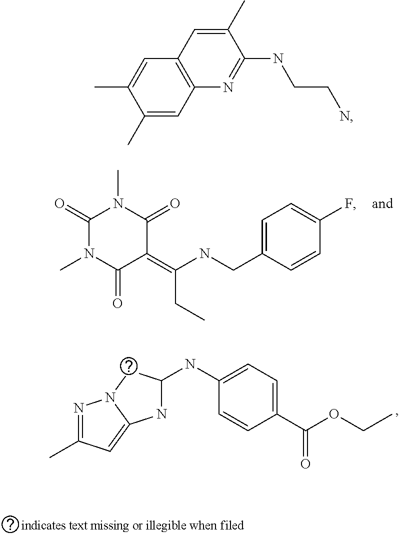
and pharmaceutically acceptable salts thereof.

18. The method of claim 17, wherein the compound is selected from the group consisting of 5-fluorouracil, Clostridium difficile Toxin B, 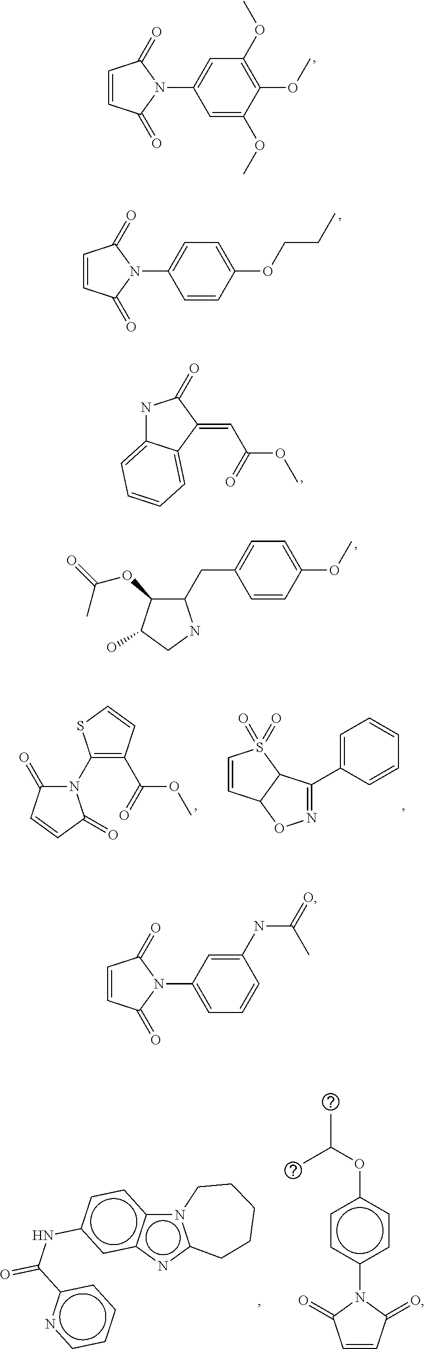 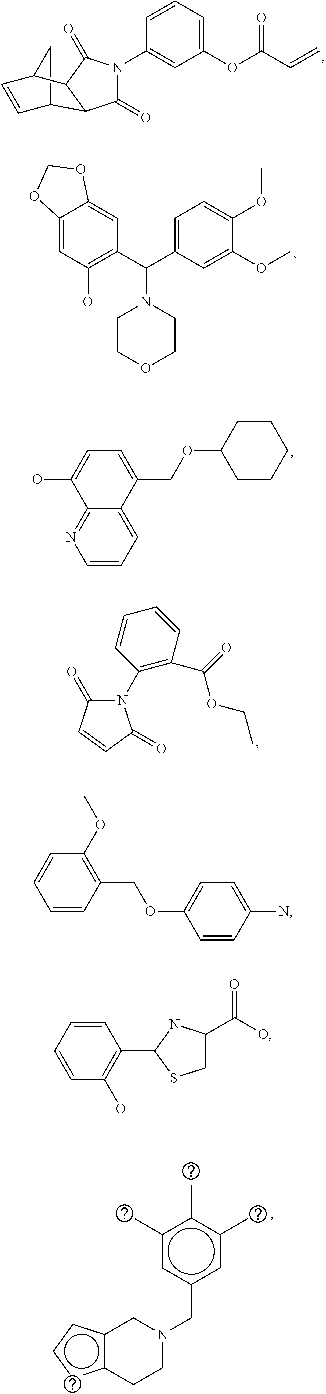 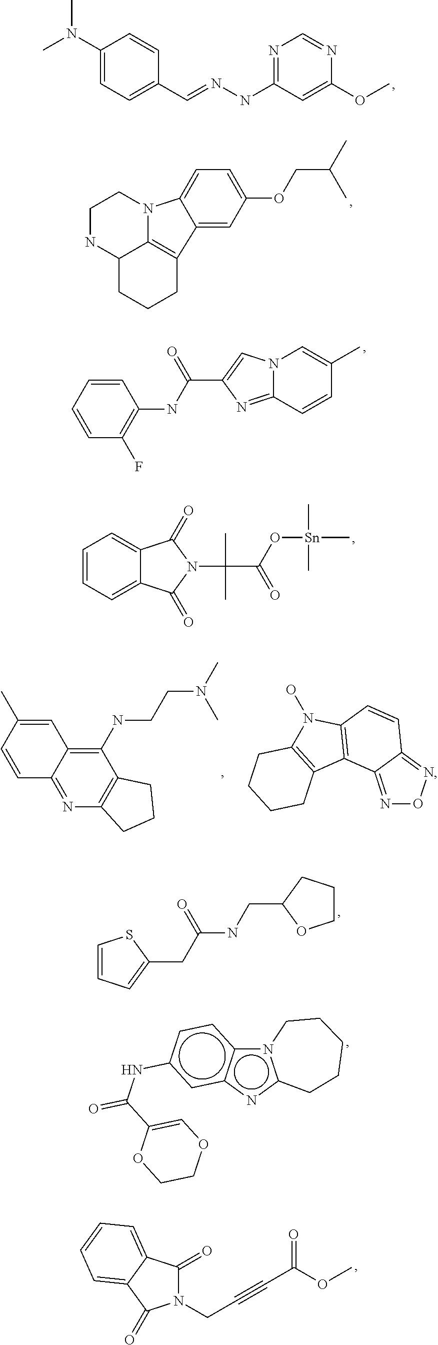 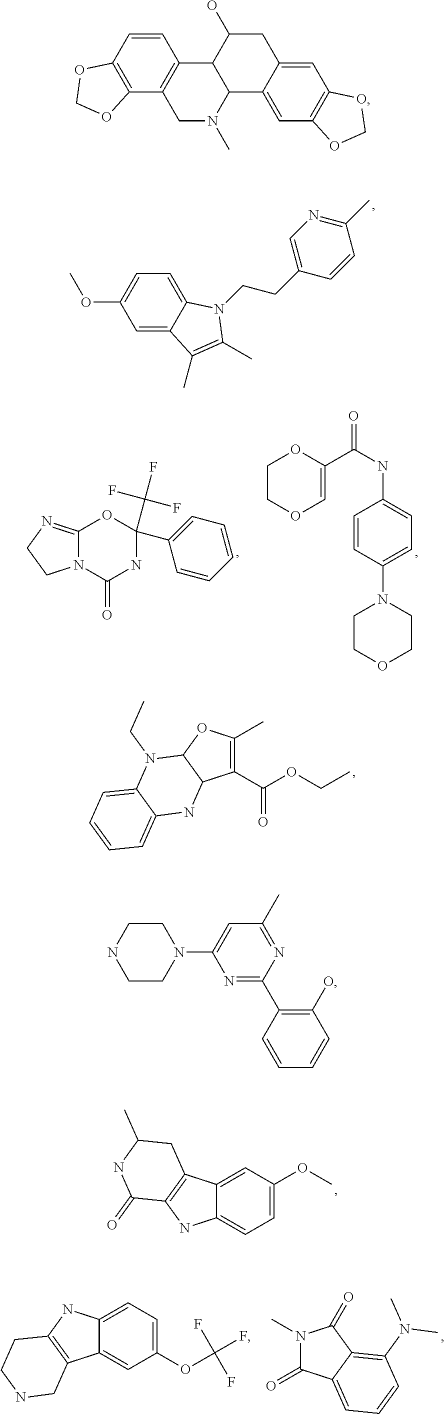 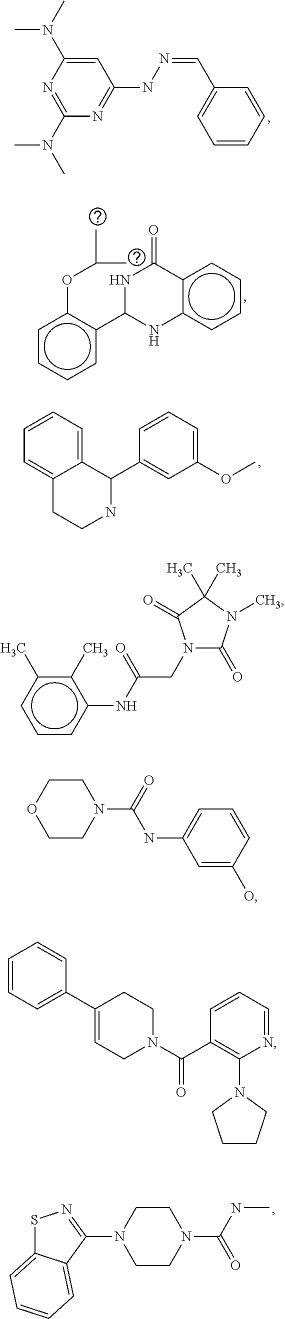 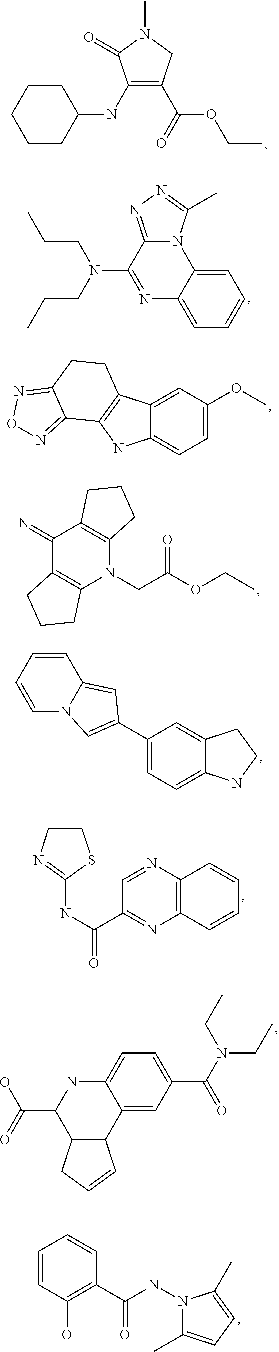 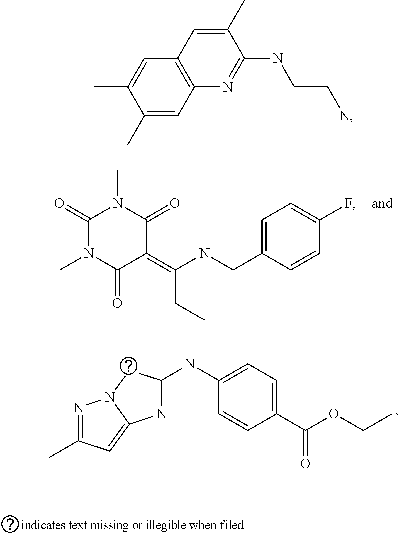
and pharmaceutically acceptable salts thereof.

19. The method of claim 18, wherein the compound is selected from the group consisting of Clostridium difficile Toxin B, 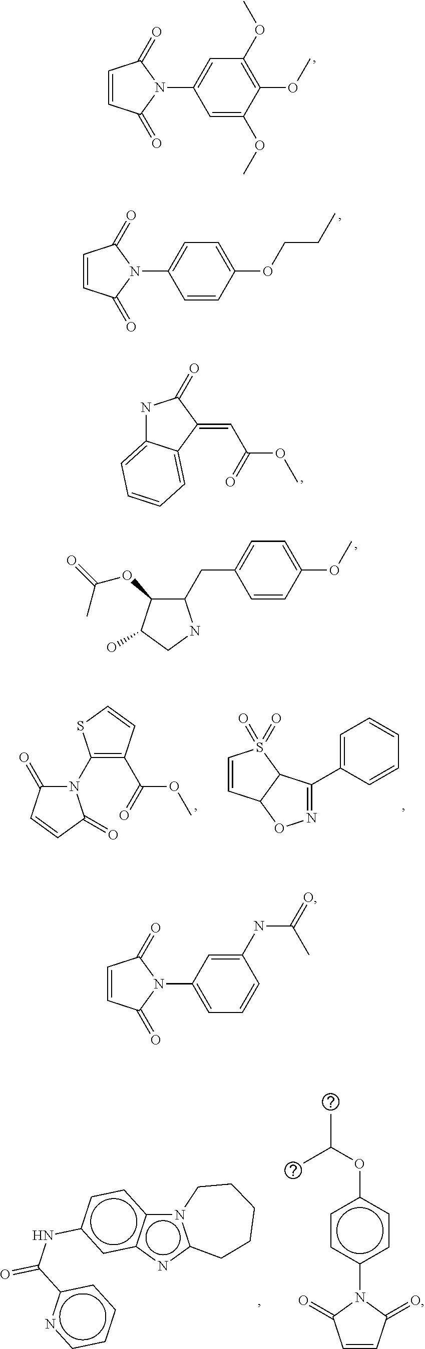 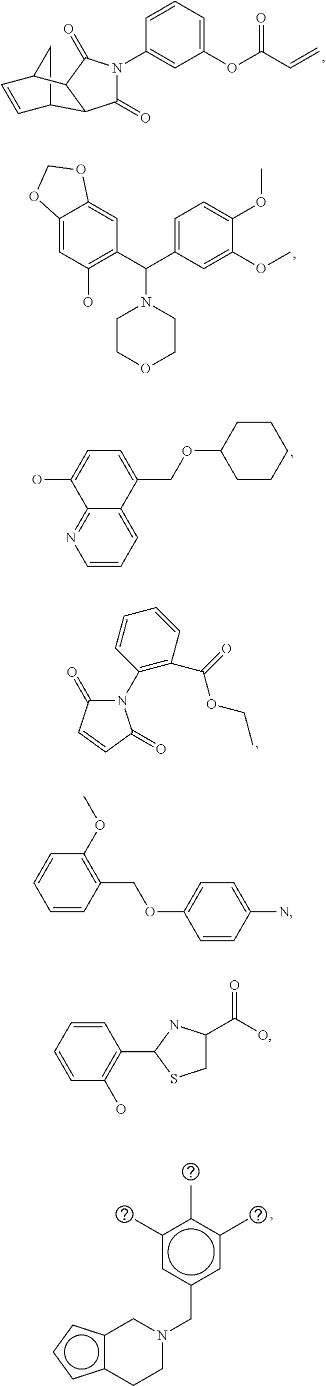 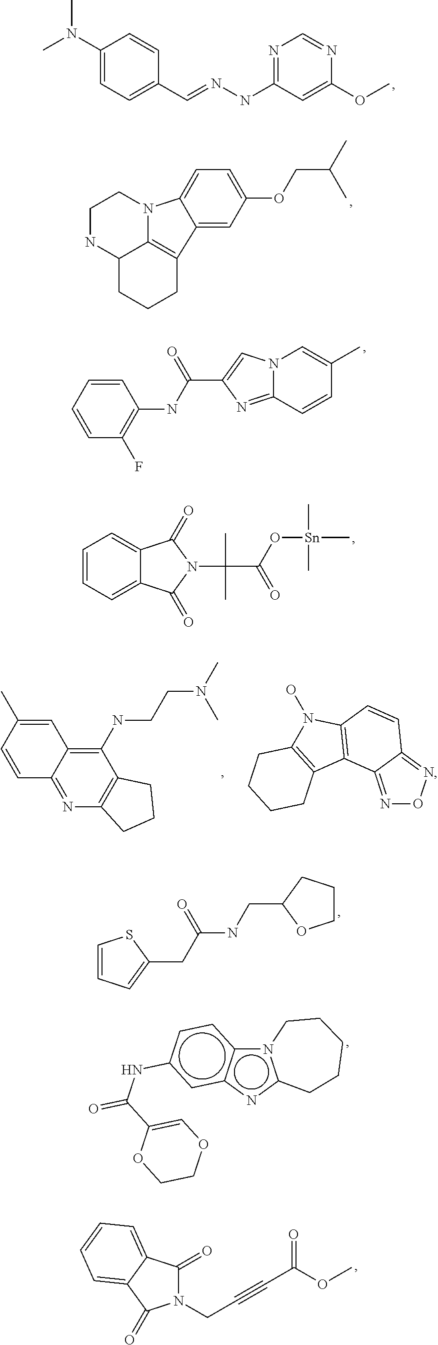 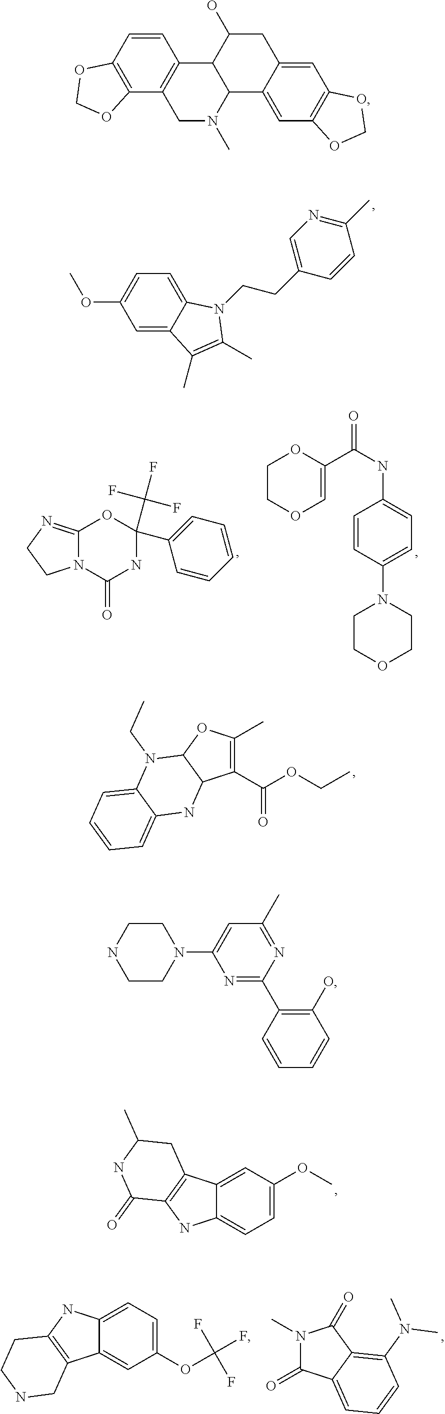 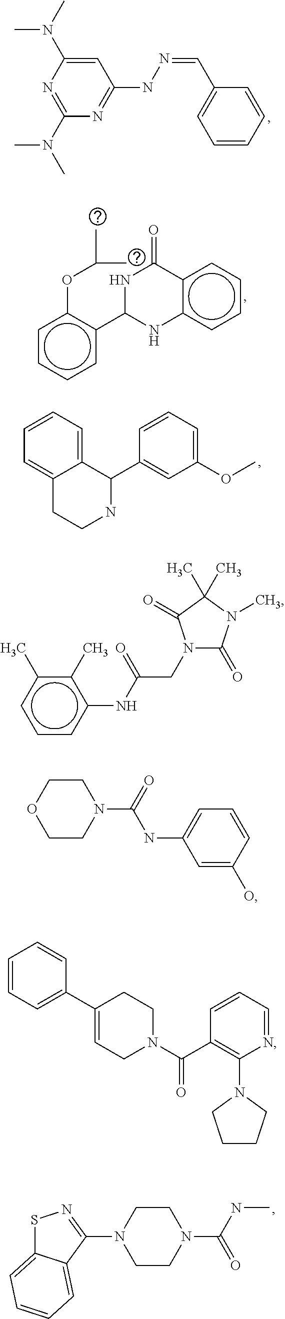 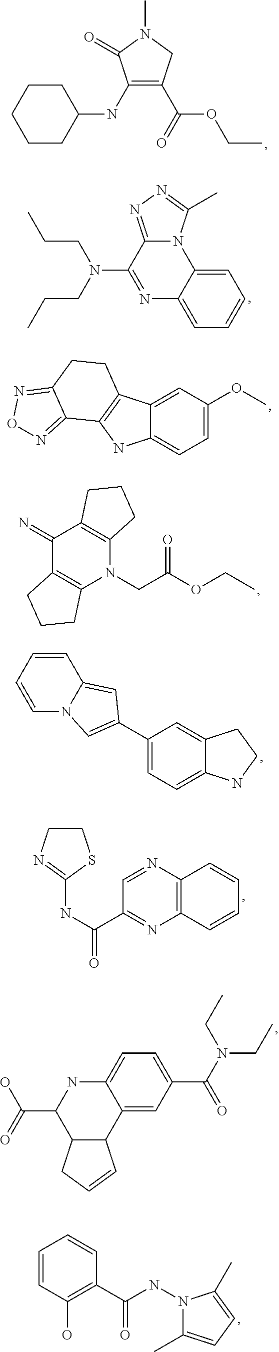 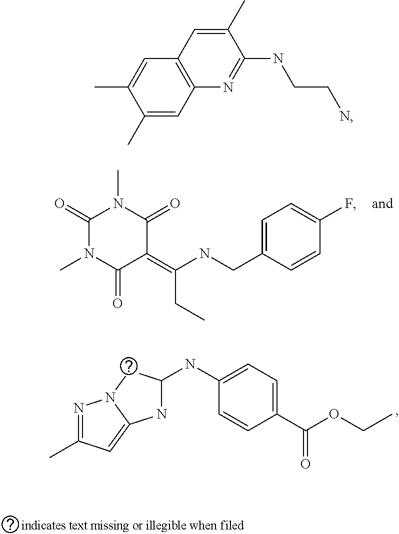
and pharmaceutically acceptable salts thereof.

20. The method of claim 17, wherein the MGES protein is C/EPB or Stat3.

21. A method for detecting the presence of or a predisposition to a nervous system cancer in a human subject, the method comprising:
(a) obtaining a biological sample from a subject; and
(b) detecting whether or not there is an alteration in the expression of a Mesenchymal-Gene-Expression-Signature (MGES) gene in the subject as compared to a subject not afflicted with a nervous system cancer.

22. The method of claim 21, wherein the MGES gene comprises Stat3, C/EBPβ, C/EBPδ, RunX1, FosL2, bHLH-B2, ZNF238, or a combination thereof.

23. The method of claim 21, wherein the detecting comprises detecting in the sample whether there is an increase in a MGES mRNA, a MGES polypeptide, or a combination thereof.

24. The method of claim 23, wherein the MGES gene comprises Stat3, C/EBPβ, C/EBPδ, RunX1, FosL2, bHLH-B2, or a combination thereof.

25. The method of claim 21, wherein the detecting comprises detecting in the sample whether there is a decrease in a MGES mRNA, a MGES polypeptide, or a combination thereof.

26. The method of claim 25, wherein the MGES gene comprises ZNF238.

27. The method of claim 21, wherein the nervous system cancer comprises a glioma.

28. The method of claim 27, wherein the glioma comprises an astrocytoma, a Glioblastoma Multiforme, an oligodendroglioma, an ependymoma, or a combination thereof.

29. A method for inhibiting proliferation of a nervous system tumor cell or for promoting differentiation of a nervous system tumor cell, the method comprising decreasing the expression of a Mesenchymal-Gene-Expression-Signature (MGES) molecule in a nervous system tumor cell, thereby inhibiting proliferation or promoting differentiation.

30. The method of claim 29, wherein the proliferation comprises cell invasion, cell migration, or a combination thereof.

31. A method for inhibiting angiogenesis in a nervous system tumor, the method comprising decreasing the expression of a Mesenchymal-Gene-Expression-Signature (MGES) molecule in a nervous system tumor cell, thereby inhibiting angiogenesis.

32. A method for treating a nervous system tumor in a subject, the method comprising administering to a nervous system tumor cell in the subject an effective amount of a composition that decreases the expression of a Mesenchymal-Gene-Expression-Signature (MGES) molecule in a nervous system tumor cell, thereby treating nervous system tumor in the subject.

33. A method for identifying a compound that binds to a Mesenchymal-Gene-Expression-Signature (MGES) protein, the method comprising:
a) providing an electronic library of test compounds;
b) providing atomic coordinates for at least 20 amino acid residues for the binding pocket of the MGES protein, wherein the coordinates have a root mean square deviation therefrom, with respect to at least 50% of Cα atoms, of not greater than about 5 Å, in a computer readable format;
c) converting the atomic coordinates into electrical signals readable by a computer processor to generate a three dimensional model of the MGES protein;
d) performing a data processing method, wherein electronic test compounds from the library are superimposed upon the three dimensional model of the MGES protein; and
e) determining which test compound fits into the binding pocket of the three dimensional model of the MGES protein,
thereby identifying which compound binds to the Mesenchymal-Gene-Expression-Signature (MGES) protein.

34. The method of claim 33, further comprising:
f) obtaining or synthesizing the compound determined to bind to the Mesenchymal-Gene-Expression-Signature (MGES) protein or to modulate MGES protein activity;
g) contacting the MGES protein with the compound under a condition suitable for binding; and
h) determining whether the compound modulates MGES protein activity using a diagnostic assay.

35. The method of claim 33, wherein the MGES protein comprises Stat3, C/EBPβ, C/EBPδ, RunX1, FosL2, bHLH-B2, ZNF238

36. The method of claim 33, wherein the compound is a MGES antagonist or MGES agonist.

37. The method of claim 36, wherein the antagonist decreases MGES protein or RNA expression or MGES activity by at least about 10%, at least about 20%, at least about 30%, at least about 40%, at least about 50%, at least about 60%, at least about 70%, at least about 75%, at least about 80%, at least about 90%, at least about 95%, at least about 99%, or 100%.

38. The method of claim 36, wherein the antagonist is directed to Stat3, C/EBPβ, C/EBPδ, RunX1, FosL2, bHLH-B2 or a combination thereof.

39. The method of claim 36, wherein the agonist increases MGES protein or RNA expression or MGES activity by at least about 10%, at least about 20%, at least about 30%, at least about 40%, at least about 50%, at least about 60%, at least about 70%, at least about 75%, at least about 80%, at least about 90%, at least about 95%, at least about 99%, or 100%.

40. The method of claim 36, wherein the agonist is directed to ZNF238.

41. A compound identified by the method of claim 33, wherein the compound binds to the active site of MGES.

42. A method for decreasing MGES gene expression in a subject having a nervous system cancer, the method comprising:
a) administering to the subject an effective amount of a composition comprising a MGES inhibitor compound,
thereby decreasing MGES expression in the subject.

43. The method of claim 33 or claim 42, wherein the compound comprises an antibody that specifically binds to a MGES protein or a fragment thereof; an antisense RNA or antisense DNA that inhibits expression of MGES polypeptide; a siRNA that specifically targets a MGES gene; a shRNA that specifically targets a MGES gene; or a combination thereof.

44. A diagnostic kit for determining whether a sample from a subject exhibits increased or decreased expression of at least 2 or more MGES genes, the kit comprising nucleic acid primers that specifically hybridize to an MGES gene, wherein the primer will prime a polymerase reaction only when a nucleic acid sequence comprising any one of SEQ ID NOS: 232, 234, 236, 238, 240, 242, or 244 is present.

45. The kit of claim 44, wherein the MGES gene is Stat3, C/EBPβ, C/EBPδ, RunX1, FosL2, bHLH-B2, ZNF238, or a combination thereof.


SEQUENCE LISTING (248 sequences)

SEQ ID NO: 1 - DNA, 30 bp, Artificial Sequence
  Description of Artificial Sequence Synthetic primer
gccttggaattcatggaagtggccaacttc

SEQ ID NO: 2 - DNA, 32 bp, Artificial Sequence
  Description of Artificial Sequence Synthetic primer
gccttgtctagacggcagtgaccggccgaggc

SEQ ID NO: 3 - DNA, 58 bp, Artificial Sequence
  Description of Artificial Sequence Synthetic oligonucleotide
ccggcatcgactacaaacggaacttctcgagaagttccgtttgtagtcgatgtttttg

SEQ ID NO: 4 - DNA, 58 bp, Artificial Sequence
  Description of Artificial Sequence Synthetic oligonucleotide
ccggcctgagttgaattatcagcttctcgagaagctgataattcaactcaggtttttg

SEQ ID NO: 5 - DNA, 20 bp, Artificial Sequence
  Description of Artificial Sequence Synthetic primer
atctggatggcattttcagg

SEQ ID NO: 6 - DNA, 20 bp, Artificial Sequence
  Description of Artificial Sequence Synthetic primer
ggggtaacattcgcagttgt

SEQ ID NO: 7 - DNA, 20 bp, Artificial Sequence
  Description of Artificial Sequence Synthetic primer
cctcaccatctctcctttgc

SEQ ID NO: 8 - DNA, 20 bp, Artificial Sequence
  Description of Artificial Sequence Synthetic primer
gggtcccaaacacttgagag

SEQ ID NO: 9 - DNA, 20 bp, Artificial Sequence
  Description of Artificial Sequence Synthetic primer
ctgaggtctcttgccgaatc

SEQ ID NO: 10 - DNA, 20 bp, Artificial Sequence
  Description of Artificial Sequence Synthetic primer
tgtcgatgtgatcgttgctt

SEQ ID NO: 11 - DNA, 20 bp, Artificial Sequence
  Description of Artificial Sequence Synthetic primer
ggtgggtggatgagtaatgc

SEQ ID NO: 12 - DNA, 20 bp, Artificial Sequence
  Description of Artificial Sequence Synthetic primer
ccctgcttacctctggtgtc

SEQ ID NO: 13 - DNA, 18 bp, Artificial Sequence
  Description of Artificial Sequence Synthetic primer
gcgctcagcctttctctg

SEQ ID NO: 14 - DNA, 19 bp, Artificial Sequence
  Description of Artificial Sequence Synthetic primer
ggagcagggagtccaagtc

SEQ ID NO: 15 - DNA, 20 bp, Artificial Sequence
  Description of Artificial Sequence Synthetic primer
tgggtggggtgtttcattat

SEQ ID NO: 16 - DNA, 20 bp, Artificial Sequence
  Description of Artificial Sequence Synthetic primer
gaacaaatgctacggggaaa

SEQ ID NO: 17 - DNA, 20 bp, Artificial Sequence
  Description of Artificial Sequence Synthetic primer
tagatcactcggggttgtcc

SEQ ID NO: 18 - DNA, 20 bp, Artificial Sequence
  Description of Artificial Sequence Synthetic primer
actgctctcagaggctaccg

SEQ ID NO: 19 - DNA, 19 bp, Artificial Sequence
  Description of Artificial Sequence Synthetic primer
ccagtgaggtgccaaatgt

SEQ ID NO: 20 - DNA, 19 bp, Artificial Sequence
  Description of Artificial Sequence Synthetic primer
gacgccctgagctctgtct

SEQ ID NO: 21 - DNA, 20 bp, Artificial Sequence
  Description of Artificial Sequence Synthetic primer
tttgggcgtatttctccttg

SEQ ID NO: 22 - DNA, 20 bp, Artificial Sequence
  Description of Artificial Sequence Synthetic primer
agaaggcaacgagttgagga

SEQ ID NO: 23 - DNA, 20 bp, Artificial Sequence
  Description of Artificial Sequence Synthetic primer
agaaggcaacgagttgagga

SEQ ID NO: 24 - DNA, 20 bp, Artificial Sequence
  Description of Artificial Sequence Synthetic primer
tttgggcgtatttctccttg

SEQ ID NO: 25 - DNA, 20 bp, Artificial Sequence
  Description of Artificial Sequence Synthetic primer
gcagcagctgtagcagtgag

SEQ ID NO: 26 - DNA, 20 bp, Artificial Sequence
  Description of Artificial Sequence Synthetic primer
gccaaggatacaggcaacat

SEQ ID NO: 27 - DNA, 19 bp, Artificial Sequence
  Description of Artificial Sequence Synthetic primer
aggggcatagcctgtctgt

SEQ ID NO: 28 - DNA, 20 bp, Artificial Sequence
  Description of Artificial Sequence Synthetic primer
caggcctgtttacggtctgt

SEQ ID NO: 29 - DNA, 20 bp, Artificial Sequence
  Description of Artificial Sequence Synthetic primer
cccaggtggatttttgtctg

SEQ ID NO: 30 - DNA, 20 bp, Artificial Sequence
  Description of Artificial Sequence Synthetic primer
acaatggtgccgttcttttc

SEQ ID NO: 31 - DNA, 20 bp, Artificial Sequence
  Description of Artificial Sequence Synthetic primer
tgcgagtaagttgtgctggt

SEQ ID NO: 32 - DNA, 20 bp, Artificial Sequence
  Description of Artificial Sequence Synthetic primer
cagcatgccgagttaaggat

SEQ ID NO: 33 - DNA, 18 bp, Artificial Sequence
  Description of Artificial Sequence Synthetic primer
ttcccatggggtgacatc

SEQ ID NO: 34 - DNA, 20 bp, Artificial Sequence
  Description of Artificial Sequence Synthetic primer
cagaggctggggtttctttc

SEQ ID NO: 35 - DNA, 20 bp, Artificial Sequence
  Description of Artificial Sequence Synthetic primer
tgaccccgagtattgtttgg

SEQ ID NO: 36 - DNA, 20 bp, Artificial Sequence
  Description of Artificial Sequence Synthetic primer
ggggtgttggtagcagagaa

SEQ ID NO: 37 - DNA, 20 bp, Artificial Sequence
  Description of Artificial Sequence Synthetic primer
caggagggagctgtatcagg

SEQ ID NO: 38 - DNA, 19 bp, Artificial Sequence
  Description of Artificial Sequence Synthetic primer
aggacttgggcacagaagc

SEQ ID NO: 39 - DNA, 19 bp, Artificial Sequence
  Description of Artificial Sequence Synthetic primer
gcaagggtccttttgtgct

SEQ ID NO: 40 - DNA, 20 bp, Artificial Sequence
  Description of Artificial Sequence Synthetic primer
gctcatccgaaaatcctcaa

SEQ ID NO: 41 - DNA, 20 bp, Artificial Sequence
  Description of Artificial Sequence Synthetic primer
cgcaaccactttgttttacg

SEQ ID NO: 42 - DNA, 20 bp, Artificial Sequence
  Description of Artificial Sequence Synthetic primer
gctgagggcacaaggaatta

SEQ ID NO: 43 - DNA, 20 bp, Artificial Sequence
  Description of Artificial Sequence Synthetic primer
cggctccgtcctttgataac

SEQ ID NO: 44 - DNA, 20 bp, Artificial Sequence
  Description of Artificial Sequence Synthetic primer
agctcccacttcagatgagc

SEQ ID NO: 45 - DNA, 20 bp, Artificial Sequence
  Description of Artificial Sequence Synthetic primer
gggctcccactgattctaca

SEQ ID NO: 46 - DNA, 20 bp, Artificial Sequence
  Description of Artificial Sequence Synthetic primer
atggtttcgggatgattcaa

SEQ ID NO: 47 - DNA, 20 bp, Artificial Sequence
  Description of Artificial Sequence Synthetic primer
gggctagtctagggggaaga

SEQ ID NO: 48 - DNA, 20 bp, Artificial Sequence
  Description of Artificial Sequence Synthetic primer
ggggttctagggagtttgga

SEQ ID NO: 49 - DNA, 21 bp, Artificial Sequence
  Description of Artificial Sequence Synthetic primer
tgtgctgtcatccagagtttg

SEQ ID NO: 50 - DNA, 20 bp, Artificial Sequence
  Description of Artificial Sequence Synthetic primer
gggtctagtgctgctgatga

SEQ ID NO: 51 - DNA, 19 bp, Artificial Sequence
  Description of Artificial Sequence Synthetic primer
cattggctctccacaccag

SEQ ID NO: 52 - DNA, 20 bp, Artificial Sequence
  Description of Artificial Sequence Synthetic primer
acatgtgtgtgcatgtgctg

SEQ ID NO: 53 - DNA, 19 bp, Artificial Sequence
  Description of Artificial Sequence Synthetic primer
cgcaacaccctgaacactc

SEQ ID NO: 54 - DNA, 18 bp, Artificial Sequence
  Description of Artificial Sequence Synthetic primer
cacttccctgcacggtct

SEQ ID NO: 55 - DNA, 20 bp, Artificial Sequence
  Description of Artificial Sequence Synthetic primer
tgggataactggcacaacct

SEQ ID NO: 56 - DNA, 23 bp, Artificial Sequence
  Description of Artificial Sequence Synthetic primer
tcaggacaattttcacattgatt

SEQ ID NO: 57 - DNA, 18 bp, Artificial Sequence
  Description of Artificial Sequence Synthetic primer
ctggctggtcgtgggtag

SEQ ID NO: 58 - DNA, 26 bp, Artificial Sequence
  Description of Artificial Sequence Synthetic primer
gggagcataatttaacctagaaaaag

SEQ ID NO: 59 - DNA, 20 bp, Artificial Sequence
  Description of Artificial Sequence Synthetic primer
accccagctcagcagataac

SEQ ID NO: 60 - DNA, 20 bp, Artificial Sequence
  Description of Artificial Sequence Synthetic primer
acctctctgccactcctagc

SEQ ID NO: 61 - DNA, 20 bp, Artificial Sequence
  Description of Artificial Sequence Synthetic primer
tcctcataaggaccctgtgg

SEQ ID NO: 62 - DNA, 20 bp, Artificial Sequence
  Description of Artificial Sequence Synthetic primer
tgtagcggaagtcagggaac

SEQ ID NO: 63 - DNA, 22 bp, Artificial Sequence
  Description of Artificial Sequence Synthetic primer
aagttgtccatttagggggaat

SEQ ID NO: 64 - DNA, 20 bp, Artificial Sequence
  Description of Artificial Sequence Synthetic primer
tggcctcgatacaattttcc

SEQ ID NO: 65 - DNA, 20 bp, Artificial Sequence
  Description of Artificial Sequence Synthetic primer
taggcgctgcactagttgat

SEQ ID NO: 66 - DNA, 20 bp, Artificial Sequence
  Description of Artificial Sequence Synthetic primer
cagccaaaggcatcctgtat

SEQ ID NO: 67 - DNA, 20 bp, Artificial Sequence
  Description of Artificial Sequence Synthetic primer
ggtcatcctgctttgaggaa

SEQ ID NO: 68 - DNA, 18 bp, Artificial Sequence
  Description of Artificial Sequence Synthetic primer
gcgggctcagagttccag

SEQ ID NO: 69 - DNA, 20 bp, Artificial Sequence
  Description of Artificial Sequence Synthetic primer
cgcttcctaaacctcccaga

SEQ ID NO: 70 - DNA, 20 bp, Artificial Sequence
  Description of Artificial Sequence Synthetic primer
ccttcgaacttgctttgcat

SEQ ID NO: 71 - DNA, 20 bp, Artificial Sequence
  Description of Artificial Sequence Synthetic primer
gcagccacctagacttaccg

SEQ ID NO: 72 - DNA, 20 bp, Artificial Sequence
  Description of Artificial Sequence Synthetic primer
ctccgggacacctgtgtatt

SEQ ID NO: 73 - DNA, 20 bp, Artificial Sequence
  Description of Artificial Sequence Synthetic primer
ctgaggagtggatgcatgtg

SEQ ID NO: 74 - DNA, 20 bp, Artificial Sequence
  Description of Artificial Sequence Synthetic primer
actgaggggagaagcacaga

SEQ ID NO: 75 - DNA, 20 bp, Artificial Sequence
  Description of Artificial Sequence Synthetic primer
aagctgaggcacagtggttt

SEQ ID NO: 76 - DNA, 20 bp, Artificial Sequence
  Description of Artificial Sequence Synthetic primer
cacccacctcctgacactct

SEQ ID NO: 77 - DNA, 20 bp, Artificial Sequence
  Description of Artificial Sequence Synthetic primer
ctgggttgtctgggttcatt

SEQ ID NO: 78 - DNA, 20 bp, Artificial Sequence
  Description of Artificial Sequence Synthetic primer
tattgacactgcccactgga

SEQ ID NO: 79 - DNA, 20 bp, Artificial Sequence
  Description of Artificial Sequence Synthetic primer
caccctttcaattggaccac

SEQ ID NO: 80 - DNA, 20 bp, Artificial Sequence
  Description of Artificial Sequence Synthetic primer
tccctggtgcttagagatgg

SEQ ID NO: 81 - DNA, 20 bp, Artificial Sequence
  Description of Artificial Sequence Synthetic primer
ccgacatcaggattgaggag

SEQ ID NO: 82 - DNA, 21 bp, Artificial Sequence
  Description of Artificial Sequence Synthetic primer
ttggctctgaagcctatagca

SEQ ID NO: 83 - DNA, 20 bp, Artificial Sequence
  Description of Artificial Sequence Synthetic primer
cctgctggggaaagtacaag

SEQ ID NO: 84 - DNA, 20 bp, Artificial Sequence
  Description of Artificial Sequence Synthetic primer
aacgaggttcctgcccttat

SEQ ID NO: 85 - DNA, 20 bp, Artificial Sequence
  Description of Artificial Sequence Synthetic primer
agtcaagctgtggctgccta

SEQ ID NO: 86 - DNA, 20 bp, Artificial Sequence
  Description of Artificial Sequence Synthetic primer
actcccttaaatgcccctgt

SEQ ID NO: 87 - DNA, 19 bp, Artificial Sequence
  Description of Artificial Sequence Synthetic primer
ataagctgcgcctttgctt

SEQ ID NO: 88 - DNA, 20 bp, Artificial Sequence
  Description of Artificial Sequence Synthetic primer
aaaattcacagggctgttgc

SEQ ID NO: 89 - DNA, 20 bp, Artificial Sequence
  Description of Artificial Sequence Synthetic primer
tctctggccctgtaacttgc

SEQ ID NO: 90 - DNA, 20 bp, Artificial Sequence
  Description of Artificial Sequence Synthetic primer
aggggagagagtgaggcatt

SEQ ID NO: 91 - DNA, 20 bp, Artificial Sequence
  Description of Artificial Sequence Synthetic primer
ccacctacatctcccctgtc

SEQ ID NO: 92 - DNA, 21 bp, Artificial Sequence
  Description of Artificial Sequence Synthetic primer
cagctggaggaggtacaaaga

SEQ ID NO: 93 - DNA, 18 bp, Artificial Sequence
  Description of Artificial Sequence Synthetic primer
gccaagacgcagaaatgc

SEQ ID NO: 94 - DNA, 20 bp, Artificial Sequence
  Description of Artificial Sequence Synthetic primer
ggcagatgtggaaggtgtct

SEQ ID NO: 95 - DNA, 19 bp, Artificial Sequence
  Description of Artificial Sequence Synthetic primer
gggtcccctatggggtatt

SEQ ID NO: 96 - DNA, 19 bp, Artificial Sequence
  Description of Artificial Sequence Synthetic primer
tgatggtttggcaaagctg

SEQ ID NO: 97 - DNA, 20 bp, Artificial Sequence
  Description of Artificial Sequence Synthetic primer
gcattcccctgctcacttag

SEQ ID NO: 98 - DNA, 22 bp, Artificial Sequence
  Description of Artificial Sequence Synthetic primer
ttggttcagccagttttctatc

SEQ ID NO: 99 - DNA, 20 bp, Artificial Sequence
  Description of Artificial Sequence Synthetic primer
tccagacttgtttccccatc

SEQ ID NO: 100 - DNA, 20 bp, Artificial Sequence
  Description of Artificial Sequence Synthetic primer
gattgcagctgggagaagtg

SEQ ID NO: 101 - DNA, 19 bp, Artificial Sequence
  Description of Artificial Sequence Synthetic primer
ctgctcgaggcttggacac

SEQ ID NO: 102 - DNA, 19 bp, Artificial Sequence
  Description of Artificial Sequence Synthetic primer
ccacccccagttttctgag

SEQ ID NO: 103 - DNA, 19 bp, Artificial Sequence
  Description of Artificial Sequence Synthetic primer
ggcttgcctgggtgtttac

SEQ ID NO: 104 - DNA, 18 bp, Artificial Sequence
  Description of Artificial Sequence Synthetic primer
gggctggagctagcaagg

SEQ ID NO: 105 - DNA, 20 bp, Artificial Sequence
  Description of Artificial Sequence Synthetic primer
agggggagaagttggtaacg

SEQ ID NO: 106 - DNA, 20 bp, Artificial Sequence
  Description of Artificial Sequence Synthetic primer
tcaggggcacagagagagtc

SEQ ID NO: 107 - DNA, 20 bp, Artificial Sequence
  Description of Artificial Sequence Synthetic primer
cagccacgtgattgtctagg

SEQ ID NO: 108 - DNA, 18 bp, Artificial Sequence
  Description of Artificial Sequence Synthetic primer
atggtggtggcagagtgg

SEQ ID NO: 109 - DNA, 18 bp, Artificial Sequence
  Description of Artificial Sequence Synthetic primer
ctgcttatccccgcagtc

SEQ ID NO: 110 - DNA, 20 bp, Artificial Sequence
  Description of Artificial Sequence Synthetic primer
ggagggaaggaactgaggag

SEQ ID NO: 111 - DNA, 20 bp, Artificial Sequence
  Description of Artificial Sequence Synthetic primer
accccagactctgtccacac

SEQ ID NO: 112 - DNA, 19 bp, Artificial Sequence
  Description of Artificial Sequence Synthetic primer
agcccctttagacccacag

SEQ ID NO: 113 - DNA, 20 bp, Artificial Sequence
  Description of Artificial Sequence Synthetic primer
agccgtttcctccttagtgg

SEQ ID NO: 114 - DNA, 20 bp, Artificial Sequence
  Description of Artificial Sequence Synthetic primer
gcttccgaggagtgtgagtg

SEQ ID NO: 115 - DNA, 20 bp, Artificial Sequence
  Description of Artificial Sequence Synthetic primer
cactgatgttgttgcccaag

SEQ ID NO: 116 - DNA, 20 bp, Artificial Sequence
  Description of Artificial Sequence Synthetic primer
gagccgagtcctgtatcagc

SEQ ID NO: 117 - DNA, 21 bp, Artificial Sequence
  Description of Artificial Sequence Synthetic primer
cctgctcagtctcagaaccac

SEQ ID NO: 118 - DNA, 20 bp, Artificial Sequence
  Description of Artificial Sequence Synthetic primer
gcaccccctaacaccactac

SEQ ID NO: 119 - DNA, 20 bp, Artificial Sequence
  Description of Artificial Sequence Synthetic primer
catgcccaaatatggttgac

SEQ ID NO: 120 - DNA, 20 bp, Artificial Sequence
  Description of Artificial Sequence Synthetic primer
gccaatttaccgagagcaag

SEQ ID NO: 121 - DNA, 20 bp, Artificial Sequence
  Description of Artificial Sequence Synthetic primer
tggaggggaaagaggaagag

SEQ ID NO: 122 - DNA, 20 bp, Artificial Sequence
  Description of Artificial Sequence Synthetic primer
acctccctgaacctgagttg

SEQ ID NO: 123 - DNA, 20 bp, Artificial Sequence
  Description of Artificial Sequence Synthetic primer
acaaggcaggcattctcatc

SEQ ID NO: 124 - DNA, 20 bp, Artificial Sequence
  Description of Artificial Sequence Synthetic primer
cctgatgaaaggcaagaacg

SEQ ID NO: 125 - DNA, 20 bp, Artificial Sequence
  Description of Artificial Sequence Synthetic primer
ggacttcctgaggctgtgtc

SEQ ID NO: 126 - DNA, 19 bp, Artificial Sequence
  Description of Artificial Sequence Synthetic primer
cctggtcacatggatttgg

SEQ ID NO: 127 - DNA, 20 bp, Artificial Sequence
  Description of Artificial Sequence Synthetic primer
atctcctgcacaaggacctg

SEQ ID NO: 128 - DNA, 20 bp, Artificial Sequence
  Description of Artificial Sequence Synthetic primer
tttctgaagtgcctgtgctg

SEQ ID NO: 129 - DNA, 20 bp, Artificial Sequence
  Description of Artificial Sequence Synthetic primer
gctctgctctgcctacatcc

SEQ ID NO: 130 - DNA, 19 bp, Artificial Sequence
  Description of Artificial Sequence Synthetic primer
aggagttgggtccccagag

SEQ ID NO: 131 - DNA, 20 bp, Artificial Sequence
  Description of Artificial Sequence Synthetic primer
cctgatacagctccctcctg

SEQ ID NO: 132 - DNA, 20 bp, Artificial Sequence
  Description of Artificial Sequence Synthetic primer
tttcgaagttgatgcaatcg

SEQ ID NO: 133 - DNA, 20 bp, Artificial Sequence
  Description of Artificial Sequence Synthetic primer
aacaagcccgtaggaacatc

SEQ ID NO: 134 - DNA, 20 bp, Artificial Sequence
  Description of Artificial Sequence Synthetic primer
actgcacctggccaactttt

SEQ ID NO: 135 - DNA, 25 bp, Artificial Sequence
  Description of Artificial Sequence Synthetic primer
tgcaaagaaaagaatacacaaagga

SEQ ID NO: 136 - DNA, 19 bp, Artificial Sequence
  Description of Artificial Sequence Synthetic primer
gccgaggtgaagaaacccc

SEQ ID NO: 137 - DNA, 22 bp, Artificial Sequence
  Description of Artificial Sequence Synthetic primer
catcgcctgatataggctgaag

SEQ ID NO: 138 - DNA, 22 bp, Artificial Sequence
  Description of Artificial Sequence Synthetic primer
ccaggtgaaaggggagaaaaag

SEQ ID NO: 139 - DNA, 21 bp, Artificial Sequence
  Description of Artificial Sequence Synthetic primer
ccaggttgacactccacaatg

SEQ ID NO: 140 - DNA, 22 bp, Artificial Sequence
  Description of Artificial Sequence Synthetic primer
ccttcagaaaccctacaatgcc

SEQ ID NO: 141 - DNA, 21 bp, Artificial Sequence
  Description of Artificial Sequence Synthetic primer
caaactgccttaggccaatct

SEQ ID NO: 142 - DNA, 22 bp, Artificial Sequence
  Description of Artificial Sequence Synthetic primer
ggacgtacaactggtattgtgc

SEQ ID NO: 143 - DNA, 21 bp, Artificial Sequence
  Description of Artificial Sequence Synthetic primer
cggcagtagtcacgaaggaat

SEQ ID NO: 144 - DNA, 19 bp, Artificial Sequence
  Description of Artificial Sequence Synthetic primer
tgcgcctcaagaccttcag

SEQ ID NO: 145 - DNA, 20 bp, Artificial Sequence
  Description of Artificial Sequence Synthetic primer
gagctgtcgcggatcagaaa

SEQ ID NO: 146 - DNA, 19 bp, Artificial Sequence
  Description of Artificial Sequence Synthetic primer
catcccccatcctacgtgg

SEQ ID NO: 147 - DNA, 21 bp, Artificial Sequence
  Description of Artificial Sequence Synthetic primer
ccccatagggtgagaaaacca

SEQ ID NO: 148 - DNA, 21 bp, Artificial Sequence
  Description of Artificial Sequence Synthetic primer
ctgctgtggaagctgggattc

SEQ ID NO: 149 - DNA, 21 bp, Artificial Sequence
  Description of Artificial Sequence Synthetic primer
ctcctccttgaactgctgtcg

SEQ ID NO: 150 - DNA, 21 bp, Artificial Sequence
  Description of Artificial Sequence Synthetic primer
catcccgaagcgaagtcttgg

SEQ ID NO: 151 - DNA, 22 bp, Artificial Sequence
  Description of Artificial Sequence Synthetic primer
ggctgggacagtccattctaaa

SEQ ID NO: 152 - DNA, 20 bp, Artificial Sequence
  Description of Artificial Sequence Synthetic primer
cttggttccctggcgtactc

SEQ ID NO: 153 - DNA, 21 bp, Artificial Sequence
  Description of Artificial Sequence Synthetic primer
acctgatccgtccacaaacag

SEQ ID NO: 154 - DNA, 20 bp, Artificial Sequence
  Description of Artificial Sequence Synthetic primer
cagagctttccaccgaatgg

SEQ ID NO: 155 - DNA, 19 bp, Artificial Sequence
  Description of Artificial Sequence Synthetic primer
gtccccggcagttgatgag

SEQ ID NO: 156 - DNA, 20 bp, Artificial Sequence
  Description of Artificial Sequence Synthetic primer
tgaccacagtccatgccatc

SEQ ID NO: 157 - DNA, 20 bp, Artificial Sequence
  Description of Artificial Sequence Synthetic primer
gacggacacattgggggtag

SEQ ID NO: 158 - DNA, 19 bp, Artificial Sequence
  Description of Artificial Sequence Synthetic primer
gggcctcttctgcgatttc

SEQ ID NO: 159 - DNA, 21 bp, Artificial Sequence
  Description of Artificial Sequence Synthetic primer
atccaggcaagtgcattggta

SEQ ID NO: 160 - DNA, 19 bp, Artificial Sequence
  Description of Artificial Sequence Synthetic primer
gcagtgaccaccattcctg

SEQ ID NO: 161 - DNA, 21 bp, Artificial Sequence
  Description of Artificial Sequence Synthetic primer
ggtagccagtgagctgaacac

SEQ ID NO: 162 - DNA, 21 bp, Artificial Sequence
  Description of Artificial Sequence Synthetic primer
ctgcgctaaacaactcaacga

SEQ ID NO: 163 - DNA, 20 bp, Artificial Sequence
  Description of Artificial Sequence Synthetic primer
gcagatccctttcagagcgg

SEQ ID NO: 164 - DNA, 23 bp, Artificial Sequence
  Description of Artificial Sequence Synthetic primer
gtggaaatgggagaatttgagga

SEQ ID NO: 165 - DNA, 21 bp, Artificial Sequence
  Description of Artificial Sequence Synthetic primer
ctcacacaccttgccatgttt

SEQ ID NO: 166 - DNA, 23 bp, Artificial Sequence
  Description of Artificial Sequence Synthetic primer
gaccattatgattcccagcagac

SEQ ID NO: 167 - DNA, 23 bp, Artificial Sequence
  Description of Artificial Sequence Synthetic primer
cggaagtcctcttcgatgttctc

SEQ ID NO: 168 - DNA, 19 bp, Artificial Sequence
  Description of Artificial Sequence Synthetic primer
ggagcctgtcagggctact

SEQ ID NO: 169 - DNA, 23 bp, Artificial Sequence
  Description of Artificial Sequence Synthetic primer
ccacaagaatctgtaccttctgc

SEQ ID NO: 170 - DNA, 19 bp, Artificial Sequence
  Description of Artificial Sequence Synthetic primer
cggagacgcatcacctctg

SEQ ID NO: 171 - DNA, 21 bp, Artificial Sequence
  Description of Artificial Sequence Synthetic primer
agggagtggaggagtcattcg

SEQ ID NO: 172 - DNA, 21 bp, Artificial Sequence
  Description of Artificial Sequence Synthetic primer
aaactggaaaccggagttgtc

SEQ ID NO: 173 - DNA, 21 bp, Artificial Sequence
  Description of Artificial Sequence Synthetic primer
cgtcttggtcgttacctgagt

SEQ ID NO: 174 - DNA, 20 bp, Artificial Sequence
  Description of Artificial Sequence Synthetic primer
ctggtgtctagtcgcccatc

SEQ ID NO: 175 - DNA, 20 bp, Artificial Sequence
  Description of Artificial Sequence Synthetic primer
gggctcagtcatctgcttct

SEQ ID NO: 176 - DNA, 19 bp, Artificial Sequence
  Description of Artificial Sequence Synthetic primer
tggacagtgttcggtctgg

SEQ ID NO: 177 - DNA, 21 bp, Artificial Sequence
  Description of Artificial Sequence Synthetic primer
cctccgtatagtgccctttgg

SEQ ID NO: 178 - DNA, 20 bp, Artificial Sequence
  Description of Artificial Sequence Synthetic primer
atcgacttcagcccctacct

SEQ ID NO: 179 - DNA, 20 bp, Artificial Sequence
  Description of Artificial Sequence Synthetic primer
ggctcacgtaaccgtagtcg

SEQ ID NO: 180 - DNA, 20 bp, Artificial Sequence
  Description of Artificial Sequence Synthetic primer
tcaagaacgaaagtcggagg

SEQ ID NO: 181 - DNA, 20 bp, Artificial Sequence
  Description of Artificial Sequence Synthetic primer
ggacatctaagggcatcaca

SEQ ID NO: 182 - DNA, 21 bp, Artificial Sequence
  Description of Artificial Sequence Synthetic primer
tggcaccttggattgagagtc

SEQ ID NO: 183 - DNA, 21 bp, Artificial Sequence
  Description of Artificial Sequence Synthetic primer
gcaggaatcggctatattgct

SEQ ID NO: 184 - DNA, 22 bp, Artificial Sequence
  Description of Artificial Sequence Synthetic primer
gtacaagctggtctgctacttc

SEQ ID NO: 185 - DNA, 21 bp, Artificial Sequence
  Description of Artificial Sequence Synthetic primer
atgtgctaagcatgttgtcgc

SEQ ID NO: 186 - DNA, 21 bp, Artificial Sequence
  Description of Artificial Sequence Synthetic primer
gatgacgatatcgctgcgctg

SEQ ID NO: 187 - DNA, 21 bp, Artificial Sequence
  Description of Artificial Sequence Synthetic primer
gtacgaccagaggcatacagg

SEQ ID NO: 188 - DNA, 20 bp, Artificial Sequence
  Description of Artificial Sequence Synthetic primer
gagctgtcgcggatcagaaa

SEQ ID NO: 189 - DNA, 23 bp, Artificial Sequence
  Description of Artificial Sequence Synthetic primer
tgaccaacattgatagctcagac

SEQ ID NO: 190 - DNA, 19 bp, Artificial Sequence
  Description of Artificial Sequence Synthetic primer
gcgcaggataaccacagca

SEQ ID NO: 191 - DNA, 22 bp, Artificial Sequence
  Description of Artificial Sequence Synthetic primer
aggattgaaacatccaatgtca

SEQ ID NO: 192 - DNA, 20 bp, Artificial Sequence
  Description of Artificial Sequence Synthetic primer
gctccagaaatttggctcag

SEQ ID NO: 193 - DNA, 21 bp, Artificial Sequence
  Description of Artificial Sequence Synthetic primer
ccaccctaatcaaggaaatga

SEQ ID NO: 194 - DNA, 21 bp, Artificial Sequence
  Description of Artificial Sequence Synthetic primer
tgaaatccaggtgttgggata

SEQ ID NO: 195 - DNA, 25 bp, Artificial Sequence
  Description of Artificial Sequence Synthetic primer
tcaagatgaccaagatgtataaagg

SEQ ID NO: 196 - DNA, 20 bp, Artificial Sequence
  Description of Artificial Sequence Synthetic primer
gcagttttccagcaatgaga

SEQ ID NO: 197 - DNA, 20 bp, Artificial Sequence
  Description of Artificial Sequence Synthetic primer
ctgacattcccaaggaggag

SEQ ID NO: 198 - DNA, 20 bp, Artificial Sequence
  Description of Artificial Sequence Synthetic primer
aggtgagggactcaaactgc

SEQ ID NO: 199 - DNA, 19 bp, Artificial Sequence
  Description of Artificial Sequence Synthetic primer
atcgacttcagcccgtacc

SEQ ID NO: 200 - DNA, 20 bp, Artificial Sequence
  Description of Artificial Sequence Synthetic primer
ccgtagtcgtcggagaagag

SEQ ID NO: 201 - DNA, 20 bp, Artificial Sequence
  Description of Artificial Sequence Synthetic primer
cgccgctagaggtgaaattc

SEQ ID NO: 202 - DNA, 20 bp, Artificial Sequence
  Description of Artificial Sequence Synthetic primer
cgccgctagaggtgaaattc

SEQ ID NO: 203 - DNA, 20 bp, Artificial Sequence
  Description of Artificial Sequence Synthetic primer
ctttcgctctggtccgtctt

SEQ ID NO: 204 - DNA, 21 bp, Artificial Sequence
  Description of Artificial Sequence Synthetic primer
tctggatggattgaagggaca

SEQ ID NO: 205 - DNA, 20 bp, Artificial Sequence
  Description of Artificial Sequence Synthetic primer
ccaacacgtcctctctcacc

SEQ ID NO: 206 - DNA, 21 bp, Artificial Sequence
  Description of Artificial Sequence Synthetic primer
gaaggcttgaaccaacctacg

SEQ ID NO: 207 - DNA, 22 bp, Artificial Sequence
  Description of Artificial Sequence Synthetic primer
tgattcagacattcgttcccac

SEQ ID NO: 208 - DNA, 21 bp, Artificial Sequence
  Description of Artificial Sequence Synthetic primer
tcccagggaagacatgagatt

SEQ ID NO: 209 - DNA, 21 bp, Artificial Sequence
  Description of Artificial Sequence Synthetic primer
tgtagccaccacatagaggaa

SEQ ID NO: 210 - DNA, 21 bp, Artificial Sequence
  Description of Artificial Sequence Synthetic primer
gcacacagtagatggggaaaa

SEQ ID NO: 211 - DNA, 21 bp, Artificial Sequence
  Description of Artificial Sequence Synthetic primer
cagcagctccttaacatcagg

SEQ ID NO: 212 - DNA, 21 bp, Artificial Sequence
  Description of Artificial Sequence Synthetic primer
tgtgaccgcaaaggattctac

SEQ ID NO: 213 - DNA, 20 bp, Artificial Sequence
  Description of Artificial Sequence Synthetic primer
gcagcttcatcccgtacttg

SEQ ID NO: 214 - DNA, 21 bp, Artificial Sequence
  Description of Artificial Sequence Synthetic primer
gcatttcccgaggacttctcc

SEQ ID NO: 215 - DNA, 21 bp, Artificial Sequence
  Description of Artificial Sequence Synthetic primer
aatctgctggataccctgctc

SEQ ID NO: 216 - DNA, 20 bp, Artificial Sequence
  Description of Artificial Sequence Synthetic primer
tatcccgggaactttgacac

SEQ ID NO: 217 - DNA, 20 bp, Artificial Sequence
  Description of Artificial Sequence Synthetic primer
tgagccaggcatatctaccc

SEQ ID NO: 218 - DNA, 20 bp, Artificial Sequence
  Description of Artificial Sequence Synthetic primer
cagcagcagaaaatcattgc

SEQ ID NO: 219 - DNA, 20 bp, Artificial Sequence
  Description of Artificial Sequence Synthetic primer
ttcaggtcccgagtgttctc

SEQ ID NO: 220 - DNA, 18 bp, Artificial Sequence
  Description of Artificial Sequence Synthetic primer
cccatcgctttcaaggtg

SEQ ID NO: 221 - DNA, 21 bp, Artificial Sequence
  Description of Artificial Sequence Synthetic primer
tggtcagagtgaagcttttcc

SEQ ID NO: 222 - DNA, 21 bp, Artificial Sequence
  Description of Artificial Sequence Synthetic primer
ggcaaaaagtgcatccgtact

SEQ ID NO: 223 - DNA, 21 bp, Artificial Sequence
  Description of Artificial Sequence Synthetic primer
ccgtcggtacatactccacag

SEQ ID NO: 224 - DNA, 57 bp, Artificial Sequence
  Description of Artificial Sequence Synthetic oligonucleotide
ccgggcaaagaatcacatgccacttctcgagaagtggcatgtgattctttgcttttt

SEQ ID NO: 225 - DNA, 57 bp, Artificial Sequence
  Description of Artificial Sequence Synthetic oligonucleotide
ccggcgacttcctctccgacctcttctcgagaagaggtcggagaggaagtcgttttt

SEQ ID NO: 226 - DNA, 57 bp, Artificial Sequence
  Description of Artificial Sequence Synthetic oligonucleotide
ccggcacggcccagtgtgcaagattctcgagaatcttgcacactgggccgtgttttt

SEQ ID NO: 227 - DNA, 57 bp, Artificial Sequence
  Description of Artificial Sequence Synthetic oligonucleotide
ccgggcactaacaaacctaattgatctcgagatcaattaggtttgttagtgcttttt

SEQ ID NO: 228 - DNA, 57 bp, Artificial Sequence
  Description of Artificial Sequence Synthetic oligonucleotide
ccggcctcgaagacatcggcagaaactcgagtttctgccgatgtcttcgaggttttt

SEQ ID NO: 229 - RNA, 19 nt, Artificial Sequence
  Description of Artificial Sequence Synthetic oligonucleotide
cugcuuggcugcugcguac

SEQ ID NO: 230 - RNA, 21 nt, Artificial Sequence
  Description of Artificial Sequence Synthetic oligonucleotide
cagccucucugcagaauucaa

SEQ ID NO: 231 - protein, 769 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGAAPYLKTKFICVTPTTCSNTIDLPMSPRTLDSLMQFGNNGEGAEPSAGGQFESLTFDMELTSECATSPM

SEQ ID NO: 232 - DNA, 4978 bp, Homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 233 - protein, 345 aa, Homo sapiens
MQRLVAWDPACLPLPPPPPAFKSMEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAAPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 234 - DNA, 1837 bp, Homo sapiens
gagccgcgcacgggactgggaaggggacccacccgagggtccagccaccagccccctcactaatagcggccaccccggcagcggcggcagcagcagcagcgacgcagcggcgacagctcagagcagggaggccgcgccacctgcgggccggccggagcgggcagccccaggccccctccccgggcacccgcgttcatgcaacgcctggtggcctgggacccagcatgtctccccctgccgccgccgccgcctgcctttaaatccatggaagtggccaacttctactacgaggcggactgcttggctgctgcgtacggcggcaaggcggcccccgcggcgccccccgcggccagacccgggccgcgcccccccgccggcgagctgggcagcatcggcgaccacgagcgcgccatcgacttcagcccgtacctggagccgctgggcgcgccgcaggccccggcgcccgccacggccacggacaccttcgaggcggctccgcccgcgcccgcccccgcgcccgcctcctccgggcagcaccacgacttcctctccgacctcttctccgacgactacgggggcaagaactgcaagaagccggccgagtacggctacgtgagcctggggcgcctgggggccgccaagggcgcgctgcaccccggctgcttcgcgcccctgcacccaccgcccccgccgccgccgccgcccgccgagctcaaggcggagccgggcttcgagcccgcggactgcaagcggaaggaggaggccggggcgccgggcggcggcgcaggcatggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcgcccccgaccgcctgctacgcgggggccgcgccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctagcgcggcccccgcgcgcgtccccctgccggccggggctgagactccggggagcgcccgcgcccgcgccctcgcccccgcccccggcggcgccggcaaaactttggcactggggcacttggcagcgcggggagcccgtcggtaattttaatattttattatatatatatatctatatttttgtccaaaccaaccgcacatgcagatggggctcccgcccgtggtgttatttaaagaagaaacgtctatgtgtacagatgaatgataaactctctgcttctccctctgcccctctccaggcgccggcgggcgggccggtttcgaagttgatgcaatcggtttaaacatggctgaacgcgtgtgtacacgggactgacgcaacccacgtgtaactgtcagccgggccctgagtaatcgcttaaagatgttcctacgggcttgttgctgttgatgttttgttttgttttgttttttggtctttttttgtattataaaaaataatctatttctatgagaaaagaggcgtctgtatattttgggaatcttttccgtttcaagcattaagaacacttttaataaacttttttttgagaatggttacaaagcc

SEQ ID NO: 235 - protein, 269 aa, Homo sapiens
MSAALFSLDGPARGAPWPAEPAPFYEPGRAGKPGRGAEPGALGEPGAAAPAMYDDESAIDFSAYIDSMAAVPTLELCHDELFADLFNSNHKAGGAGPLELLPGGPARPLGPGPAAPRLLKREPDWGDGDAPGSLLPAQVAACAQTVVSLAAAGQPTPPTSPEPPRSSPRQTPAPGPAREKSAGKRGPDRGSPEYRQRRERNNIAVRKSRDKAKRRNQEMQQKLVELSAENEKLHQRVEQLTRDLAGLRQFFKQLPSPPFLPAAGTADCR

SEQ ID NO: 236 - DNA, 1269 bp, Homo sapiens
aggtgacagcctcgcttggacgcagagcccggcccgacgccgccatgagcgccgcgctcttcagcctggacggcccggcgcgcggcgcgccctggcctgcggagcctgcgcccttctacgaaccgggccgggcgggcaagccgggccgcggggccgagccaggggccctaggcgagccaggcgccgccgcccccgccatgtacgacgacgagagcgccatcgacttcagcgcctacatcgactccatggccgccgtgcccaccctggagctgtgccacgacgagctcttcgccgacctcttcaacagcaatcacaaggcgggcggcgcggggcccctggagcttcttcccggcggccccgcgcgccccttgggcccgggccctgccgctccccgcctgctcaagcgcgagcccgactggggcgacggcgacgcgcccggctcgctgttgcccgcgcaggtggccgcgtgcgcacagaccgtggtgagcttggcggccgcagggcagcccaccccgcccacgtcgccggagccgccgcgcagcagccccaggcagacccccgcgcccggccccgcccgggagaagagcgccggcaagaggggcccggaccgcggcagccccgagtaccggcagcggcgcgagcgcaacaacatcgccgtgcgcaagagccgcgacaaggccaagcggcgcaaccaggagatgcagcagaagttggtggagctgtcggctgagaacgagaagctgcaccagcgcgtggagcagctcacgcgggacctggccggcctccggcagttcttcaagcagctgcccagcccgcccttcctgccggccgccgggacagcagactgccggtaacgcgcggccggggcgggagagactcagcaacgacccatacctcagacccgacggcccggagcggagcgcgccctgccctggcgcagccagagccgccgggtgcccgctgcagtttcttgggacataggagcgcaaagaagctacagcctggacttaccaccactaaactgcgagagaagctaaacgtgtttattttcccttaaattatttttgtaatggtagctttttctacatcttactcctgttgatgcagctaaggtacatttgtaaaaagaaaaaaaaccagacttttcagacaaaccctttgtattgtagataagaggaaaagactgagcatgctcacttttttatattaatttttacagtatttgtaagaataaagcagcatttgaaatcgaaaaaaaaaaaaaaaaaa

SEQ ID NO: 237 - protein, 453 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 238 - DNA, 7274 bp, Homo sapiens
catagagccagcgggcgcgggcgggacgggcgccccgcggccggacccagccagggcaccacgctgcccggccctgcgccgccaggcacttctttccggggctcctagggacgccagaaggaagtcaacctctgctgcttctccttggcctgcgttggaccttcctttttttgttgtttttttttgtttttcccctttcttccttttgaattaactggcttcttggctggatgttttcaacttctttcctggctgcgaacttttccccaattgttttccttttacaacagggggagaaagtgctctgtggtccgaggcgagccgtgaagttgcgtgtgcgtggcagtgtgcgtggcaggatgtgcgtgcgtgtgtaacccgagccgcccgatctgtttcgatctgcgccgcggagccctccctcaaggcccgctccacctgctgcggttacgcggcgctcgtgggtgttcgtgcctcggagcagctaaccggcgggtgctgggcgacggtggaggagtatcgtctcgctgctgcccgagtcagggctgagtcacccagctgatgtagacagtggctgccttccgaagagtgcgtgtttgcatgtgtgtgactctgcggctgctcaactcccaacaaaccagaggaccagccacaaacttaaccaacatccccaaacccgagttcacagatgtgggagagctgtagaaccctgagtgtcatcgactgggccttcttatgattgttgttttaagattagctgaagatctctgaaacgctgaattttctgcactgagcgttttgacagaattcattgagagaacagagaacatgacaagtacttctagctcagcactgctccaactactgaagctgattttcaaggctacttaaaaaaatctgcagcgtacattaatggatttctgttgtgtttaaattctccacagattgtattgtaaatattttatgaagtagagcatatgtatatatttatatatacgtgcacatacattagtagcactacctttggaagtctcagctcttgcttttcgggactgaagccagttttgcatgataaaagtggccttgttacgggagataattgtgttctgttgggactttagacaaaactcacctgcaaaaaactgacaggcattaactactggaacttccaaataatgtgtttgctgatcgttttactcttcgcataaatattttaggaagtgtatgagaattttgccttcaggaacttttctaacagccaaagacagaacttaacctctgcaagcaagattcgtggaagatagtctccactttttaatgcactaagcaatcggttgctaggagcccatcctgggtcagaggccgatccgcagaaccagaacgttttcccctcctggactgttagtaacttagtctccctcctcccctaaccacccccgcccccccccaccccccgcagtaataaaggcccctgaacgtgtatgttggtctcccgggagctgcttgctgaagatccgcgcccctgtcgccgtctggtaggagctgtttgcagggtcctaactcaatcggcttgttgtgatgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 239 - protein, 326 aa, Homo sapiens
MYQDYPGNFDTSSRGSSGSPAHAESYSSGGGGQQKFRVDMPGSGSAFIPTINAITTSQDLQWMVQPTVITSMSNPYPRSHPYSPLPGLASVPGHMALPRPGVIKTIGTTVGRRRRDEQLSPEEEEKRRIRRERNKLAAAKCRNRRRELTEKLQAETEELEEEKSGLQKEIAELQKEKEKLEFMLVAHGPVCKISPEERRSPPAPGLQPMRSGGGSVGAVVVKQEPLEEDSPSSSSAGLDKAQRSVIKPISIAGGFYGEEPLHTPIVVTSTPAVTPGTSNLVFTYPSVLEQESPASPSESCSKAHRRSSSSGDQSSDSLNSPTLLAL

SEQ ID NO: 240 - DNA, 4015 bp, Homo sapiens
cgaacgagcggcgctcggcggggacagaaagagggagagagagagagagagagagggagaggcgcggccgggcgaggcgggcccgtccgggagcgggctccggggaaggggtgcgggtctgggcgccggagcggggagcggggccgcgtccctctcagcgccagctctacttgagccccacgagccgctgtccccctggcgcgctcggggccgcgggacgggcgcacgccgccttctcctagtcaagtatccgagccgccccgaaactcgggcggcgagtcggccacgggaagtttattctccggctccttttctaaaaggaagaaacagaagtttctcccagcggacagcttttctttccgcctttttggccctgtctgaaatcgggggtccccagggctggcaggccaggctcgctgggctcctaatcttttttttaatttccaatttttgattgggccgtgggtccccgctgagctccggctgcgcgcgggggcgggagggcgcgcgcaggggagggaccgagagacgcgccgactttttagagggagggatcgggtggacaactggtcccgcggcgctcgcagagccggaaagaagtgctgtaagggacgctcgggggacgctgttcctgaggtgtcgccgcctccctgtcctcgccctccgcggtgggggagaaacccaggagcgaagcccagagcccgcggcgcggccggcggacgaacgagcgcgcagcagccggtgcgcggccgcggcgagggcgggggaagaaaaacaccctgtttcctctccggcccccaccgcggatcatgtaccaggattatcccgggaactttgacacctcgtcccggggcagcagcggctctcctgcgcacgccgagtcctactccagcggcggcggcggccagcagaaattccgggtagatatgcctggctcaggcagtgcattcatccccaccatcaacgccatcacgaccagccaggacctgcagtggatggtgcagcccacagtgatcacctccatgtccaacccataccctcgctcgcacccctacagccccctgccgggcctggcctctgtccctggacacatggccctcccaagacctggcgtgatcaagaccattggcaccaccgtgggccgcaggaggagagatgagcagctgtctcctgaagaggaggagaagcgtcgcatccggcgggagaggaacaagctggctgcagccaagtgccggaaccgacgccgggagctgacagagaagctgcaggcggagacagaggagctggaggaggagaagtcaggcctgcagaaggagattgctgagctgcagaaggagaaggagaagctggagttcatgttggtggctcacggcccagtgtgcaagattagccccgaggagcgccgatcgcccccagcccctgggctgcagcccatgcgcagtgggggtggctcggtgggcgctgtagtggtgaaacaggagcccctggaagaggacagcccctcgtcctcgtcggcggggctggacaaggcccagcgctctgtcatcaagcccatcagcattgctgggggcttctacggtgaggagcccctgcacacccccatcgtggtgacctccacacctgctgtcactccgggcacctcgaacctcgtcttcacctatcctagcgtcctggagcaggagtcacccgcatctccctccgaatcctgctccaaggctcaccgcagaagcagtagcagcggggaccaatcatcagactccttgaactcccccactctgctggctctgtaacccagtgcacctccctccccagctccggagggggtcctcctcgctcctccttcccagggaccagcaccttcaagcgctccagggccgtgagggcaagagggggacctgccaccagggagcttcctggctctgggggacccaggtgggacttagcagtgagtattggaagacttgggttgatctcttagaagccatgggacctcctccctcattcatcttgcaagcaaatcccatttcttgaaaagccttggagaactcggtttggtagacttggacatctctctggcttctgaagagcctgaagctggcctggaccattcctgtccctttgttaccatactgtctctggagtgatggtgtccttccctgccccaccacgcatgctcagtgccttttggtttcaccttccctcgacttgaccctttcctcccccagcgtcagtttcactccctcttggtttttatcaaatttgccatgacatttcatctgggtggtctgaatattaaagctcttcatttctggagatggggcagcaggtggctcttctgctggggctgacttgtccagaaggggacaaagtgcaatacagagccttccctaccctgacgcctcccagtcatcatctccagaactcccagcggggctccctgagctctcaaggagatgctgccatcactgggaggctcagaggacccttcctgcccaccttcggagacggcttctggaggaacggcttggccagaagacagggtgtgagtgagacagtggggcacaggttgggtttgccaaacgcctaattaccaggccaggaagcatgccaacaaagccacacgggtgtcctagccagcttcccttcacctggtgtcttgagtagggcgtctcctgtaattactgccttgccattctgcccctggacccttctctccggaccagggaggcgtccctccctaggagccacacattatactccaagtccctgccgggctccgcctttcccccaccctggctctcagggtgacgccacccacagagatttaatgagcgtgggcctggaccttccccagatgctgccaggcagcccctccccaagcctcaaagaagcatttgctgaggatggagaggcaggggagggaggcgggaggccgtcactggagtggcgtctgcagcagctgctgccccagcacccgctcagcctgtcctggctgctcacctccccgcagggcaccgggcctttcctgccctctgtggtcatctgccacctgctggatcaagtgctttctcttttacactcccctgtccccaccccagtgcactcttctggcccaggcagcaagcaagctgtgaacagctggcctgagctgtcgctgtggcttgtggctcatgcgccattcctggttgtctgttgaatctttctggctgctggaattggagataggatgttttgcttcccactgcaggagagctgccccctttcacggggttggggaagggtccccctggcctccagcaggagcacagctcagcagggtccctgctgcccacccctctgagccttttctccccagggtatggctcctgctgagtttcttgtccagcagggccttgacaggaatccagggagtagctcctggccagaaccagcctctgcggggcttgtgctctgcaaagactctgctgctggggattcagctctagaggtcacagtatcctcgtttgaaagataattaagatcccccgtggagaaagcagtgacacattcacacagctgttccctcgcatgttatttcatgaacatgacctgttttcgtgcactagacacacagagtggaacagccgtatgcttaaagtacatgggccagtgggactggaagtgacctgtacaagtgatgcagaaaggagggtttcaaagaaaaaggattttgtttaaaatactttaaaaatgttatttcctgcatcccttggctgtgatgcccctctcccgatttcccaggggctctgggagggacccttctaagaagattgggcagttgggtttctggcttgagatgaatccaagcagcagaatgagccaggagtagcaggagatgggcaaagaaaactggggtgcactcagctctcacaggggtaatcatctcaagtggtatttgtagccaagtgggagctattttcttttttgtgcatatagatatttcttaaatgaaaaaaaaaaaaaaaaaaaaaaaaaa

SEQ ID NO: 241 - protein, 412 aa, Homo sapiens
MERIPSAQPPPACLPKAPGLEHGDLPGMYPAHMYQVYKSRRGIKRSEDSKETYKLPHRLIEKKRRDRINECIAQLKDLLPEHLKLTTLGHLEKAVVLELTLKHVKALTNLIDQQQQKIIALQSGLQAGELSGRNVETGQEMFCSGFQTCAREVLQYLAKHENTRDLKSSQLVTHLHRVVSELLQGGTSRKPSDPAPKVMDFKEKPSSPAKGSEGPGKNCVPVIQRTFAHSSGEQSGSDTDTDSGYGGESEKGDLRSEQPCFKSDHGRRFTMGERIGAIKQESEEPPTKKNRMQLSDDEGHFTSSDLISSPFLGPHPHQPPFCLPFYLIPPSATAYLPMLEKCWYPTSVPVLYPGLNASAAALSSFMNPDKISAPLLMPQRLPSPLPAHPSVDSSVLLQALKPIPPLNLETKD

SEQ ID NO: 242 - DNA, 3061 bp, Homo sapiens
cgcctccccgcccgccccacttctcattcacttggctcgcacggcgcagacagaccgcgcagggagcacacaccgccagtctgtgcgctgagtcggagccagaggccgcggggacaccgggccatgcacgcccccaactgaagctgcatctcaaagccgaagattccagcagcccaggggatttcaaagagctcagactcagaggaacatctgcggagagacccccgaagccctctccagggcagtcctcatccagacgctccgctagtgcagacaggagcgcgcagtggccccggctcgccgcgccatggagcggatccccagcgcgcaaccaccccccgcctgcctgcccaaagcaccgggactggagcacggagacctaccagggatgtaccctgcccacatgtaccaagtgtacaagtcaagacggggaataaagcggagcgaggacagcaaggagacctacaaattgccgcaccggctcatcgagaaaaagagacgtgaccggattaacgagtgcatcgcccagctgaaggatctcctacccgaacatctcaaacttacaactttgggtcacttggaaaaagcagtggttcttgaacttaccttgaagcatgtgaaagcactaacaaacctaattgatcagcagcagcagaaaatcattgccctgcagagtggtttacaagctggtgagctgtcagggagaaatgtcgaaacaggtcaagagatgttctgctcaggtttccagacatgtgcccgggaggtgcttcagtatctggccaagcacgagaacactcgggacctgaagtcttcgcagcttgtcacccacctccaccgggtggtctcggagctgctgcagggtggtacctccaggaagccatcagacccagctcccaaagtgatggacttcaaggaaaaacccagctctccggccaaaggttcggaaggtcctgggaaaaactgcgtgccagtcatccagcggactttcgctcactcgagtggggagcagagcggcagcgacacggacacagacagtggctatggaggagaatcggagaagggcgacttgcgcagtgagcagccgtgcttcaaaagtgaccacggacgcaggttcacgatgggagaaaggatcggcgcaattaagcaagagtccgaagaaccccccacaaaaaagaaccggatgcagctttcggatgatgaaggccatttcactagcagtgacctgatcagctccccgttcctgggcccacacccacaccagcctcctttctgcctgcccttctacctgatcccaccttcagcgactgcctacctgcccatgctggagaagtgctggtatcccacctcagtgccagtgctatacccaggcctcaacgcctctgccgcagccctctctagcttcatgaacccagacaagatctcggctcccttgctcatgccccagagactcccttctcccttgccagctcatccgtccgtcgactcttctgtcttgctccaagctctgaagccaatcccccctttaaacttagaaaccaaagactaaactctctaggggatcctgctgctttgctttccttcctcgctacttcctaaaaagcaacaaaaaagtttttgtgaatgctgcaagattgttgcattgtgtatactgagataatctgaggcatggagagcagattcagggtgtgtgtgtgtgtgtgtgtgtgtgtatgtgcgtgtgcgtgcacatgtgtgcctgcgtgttggtataggactttaaagctccttttggcatagggaagtcacgaaggattgcttgacatcaggagacttgggggggattgtagcagacgtctgggcttttccccacccagagaatagcccccttcgatacacatcagctggattttcaaaagcttcaaagtcttggtctgtgagtcactcttcagtttgggagctgggtctgtggctttgatcagaaggtactttcaaaagagggctttccagggctcagctcccaaccagctgttaggaccccacccttttgcctttattgtcgacgtgactcaccagacgtcggggagagagagcagtcagaccgagctttctgctaacatggggaggtagcaggcactggcatagcacggtagtggtttggggaggtttccgcaggtctgctccccacccctgcctcggaagaataaagagaatgtagttccctactcaggctttcgtagtgattagcttactaaggaactgaaaatgggccccttgtacaagctgagctgccccggagggagggaggagttccctgggcttctggcacctgtttctaggcctaaccattagtacttactgtgcagggaaccaaaccaaggtctgagaaatgcggacaccccgagcgagcaccccaaagtgcacaaagctgagtaaaaagctgcccccttcaaacagaactagactcagttttcaattccatcctaaaactccttttaaccaagcttagcttctcaaaggcctaaccaagccttggcaccgccagatcctttctgtaggctaattcctcttgcccaacggcatatggagtgtccttattgctaaaaaggattccgtctccttcaaagaagttttatttttggtccagagtacttgttttcccgatgtgtccagccagctccgcagcagcttttcaaaatgcactatgcctgattgctgatcgtgttttaactttttcttttcctgtttttattttggtattaagtcgttgcctttatttgtaaagctgttataaatatatattatataaatatattaaaaaggaaaatgtttcagatgtttatttgtataattacttgattcacacagtgagaaaaaatgaatgtattcctgtttttgaagagaagaataattttttttttctctagggagaggtacagtgtttatattttggagccttcctgaaggtgtaaaattgtaaatatttttatctatgagtaaatgttaagtagttgttttaaaatacttaataaaataattcttttcctgtggaagagaaaaaaaaaaaaaaaaaaaaaaaaaaaaa

SEQ ID NO: 243 - protein, 522 aa, Homo sapiens
MEFPDHSRHLLQCLSEQRHQGFLCDCTVLVGDAQFRAHRAVLASCSMYFHLFYKDQLDKRDIVHLNSDIVTAPAFALLLEFMYEGKLQFKDLPIEDVLAAASYLHMYDIVKVCKKKLKEKATTEADSTKKEEDASSCSDKVESLSDGSSHIAGDLPSDEDEGEDEKLNILPSKRDLAAEPGNMWMRLPSDSAGIPQAGGEAEPHATAAGKTVASPCSSTESLSQRSVTSVRDSADVDCVLDLSVKSSLSGVENLNSSYFSSQDVLRSNLVQVKVEKEASCDESDVGTNDYDMEHSTVKESVSTNNRVQYEPAHLAPLREDSVLRELDREDKASDDEMMTPESERVQVEGGMESSLLPYVSNILSPAGQIFMCPLCNKVFPSPHILQIHLSTHFREQDGIRSKPAADVNVPTCSLCGKTFSCMYTLKRHERTHSGEKPYTCTQCGKSFQYSHNLSRHAVVHTREKPHACKWCERRFTQSGDLYRHIRKFHCELVNSLSVKSEALSLPTVRDWTLEDSSQELWK

SEQ ID NO: 244 - DNA, 4244 bp, Homo sapiens
tttaaactgtgctttctaagcacagtcaggtagcaaaagtaataaaaaggatggttgaacaagttttcttgtatgttccaggatatgtttgggacttttctttgtttattatatgagttgttccctttgaaattaaagctattttgtaggttttgtgggacataatttgataagtagagttaattaaatttcttctggaagagatctaaattcttattcttagtgagagactgtagttaaaggaaggcttttagaacttgggttcaaggaagatggagatgcgtcggaagctctttggcgggggtgaggaagttcagaaagtgtgcattttccttctggcatttaggtcttgtccgtgtgatttggtggtgcttgggtcataagcctgattaaaattcagggacatgtaccacggcggccaaagcggaattaatttttttatatggggactggagcgctgaaaagttgttcctgaccaggctctaatgagaaattcctctctccccaggttatgaagacagtatggagtttccagaccatagtagacatttgctacagtgtctgagcgagcagagacaccagggttttctttgtgactgcactgttctggtgggagatgcccagttccgagcgcaccgagctgtactggcttcatgcagcatgtatttccacctcttttacaaggaccagctggacaaaagagacattgttcatctgaacagcgacattgttacagcccccgctttcgctctcctgcttgaattcatgtatgaagggaaactccagttcaaagacttgcccattgaagacgtgctagcagctgccagttatctccacatgtatgacattgtcaaagtctgcaaaaagaagctgaaagagaaagccaccacggaggcagacagcaccaaaaaggaagaagatgcttcaagttgttcggacaaagtcgagagtctctccgatggcagcagccacatagcaggcgatttgcccagtgatgaagatgaaggagaagatgaaaaattgaacatcctgcccagcaaaagggacttggcggccgagcctgggaacatgtggatgcgattgccctcagactcagcaggcatcccccaggctggcggagaggcagagccacacgccacagcagctggaaaaacagtagccagcccctgcagctcaacagagtctttgtcccagaggtctgtcacctccgtgagggattcggcagatgttgactgtgtgctggacctgtctgtcaagtccagcctttcaggagttgaaaatctgaacagctcttatttctcttcacaggacgtgctgagaagcaacctggtgcaggtgaaggtggagaaagaggcttcctgtgatgagagtgatgttggcactaatgactatgacatggaacatagcactgtgaaagaaagtgtgagcactaataacagggtacagtatgagccggcccatctggctcccctgagggaggactcggtcttgagggagctggaccgggaggacaaagccagtgatgatgagatgatgaccccagagagcgagcgtgtccaggtggagggaggcatggagagcagtctgctcccctacgtctccaacatcctgagccccgcgggccagatcttcatgtgccccctgtgcaacaaggtcttccccagcccccacatcctgcagatccacctgagcacgcacttccgcgagcaggacggcatccgcagcaagcccgccgccgatgtcaacgtgcccacgtgctcgctgtgtgggaagactttctcttgcatgtacaccctcaagcgccacgagaggactcactcgggggagaagccctacacatgcacccagtgcggcaagagcttccagtactcgcacaacctgagccgccatgccgtggtgcacacccgcgagaagccgcacgcctgcaagtggtgcgagcgcaggttcacgcagtccggggacctgtacagacacattcgcaagttccactgtgagttggtgaactccttgtcggtcaaaagcgaagcactgagcttgcctactgtcagagactggaccttagaagatagctctcaagaactttggaaataattttatatatatataaataatatatatatatatacatatatataaatagatctctatatagttgtggtacggtctaaaagcagtcttgtttcctggaaataaaaagttgggatattaacttgtttttgcactttagaatagcatgagaatctcactaatttagcattctgataaaagaaactttagagcaagtcagaatagagaggtgtttttcctttgaggggataggggaagtaagccaataagaaccttttaaacaaatcgtcctgtcacaaaatgctttcatatggcttaattttgtcaacactgcattgtcttttgagctcttttttcccccccaacaaagtttttttgttttttgtttttttttttaagtagaaattccctccagttttattagcctctttatatgtctcaaattgcatgaattttttctggctgttggaaacctgaatgcttttagacccaaatggaaaatttctgaaatgctggattatctatttttaaacaagcagttgacttaaaactttctgtggcaacttctggttttctgacagttcccagtgagagaaatgctgaaagtacactgggatcactgggacactgtcttatgaaggtttgcttgggatgaaaaaggatattgcagcttcagcagtgttgaactgtgtgtttaaaaatgtgaattactgttattgtatactgtaattgattacatgggctgggggggtgtcaaagaacttgacaggttgtgttgatgctcttagttgagtcttgaaaagtaaatattaacgctacagaaatgcatgagtttcaatatattttttgtctttgtttgcattgtataactttaacgagtgagtttaaaattatttaatttccttagaaaaatagcaccatttggaaaaaaaaactggtgttatgaagaacgtaaatgcactgtttttatttttattttatataatttaaattgactttcccactgtctttaagttgaaactgttaagctgaataaaaacttaagctgcaaattgataacttcgctacataacaaggaaaatataaatgtttacaaacagcttaaagatttgcatgtgcagtgtgcatttataacaaacttctaattgcacaaaacccatgccagctcagagtttaggtgtacacatttacccagttgagcgttcttagaataactactgcacaagttgacaataggtcgttctctctttttttttgtttgctccctttttctttttctccccttcctccttaccctccctcccttactctccccccccaccaccaccctccacccccaactcatgaaaagattctatggactgaaaaagccccaggctgaaaggactggactgccttgattgacatggggaagggggttagtagactatgtggattgcggcagcagaggctgcagcctaacgtgtggttttaatgaccagcacgcaaggcaaaagcattttgcacagtgtttgttttcctgtcttgcacttacaaataaggtctatgggagtagcatggaaaacgtttgctgtttttcccttttttttttaattgcttttgtttaaaatttgatcgccttaactactgtaaacatagcctatttttgtgcttaagatactgaatggaaaactccattgtgtgttgctggactgttttggaaatatttggttaaatgtgtgttaatttggctgtaatggcatttaaagcaaacaaacaaacaaaaaaagctgtgaaaatggccttggagcattatctttagttacttgaagagtttctagtttttttaaaatacagtttatgttaaaataatttttattaatttagagaagacaatcaatgtctgtgagaaaacggactttcttttggattttctttttgtggtcattgtgagtgattgctttttccttttcttagtttcacattcttcctttgttctaaaacttagactgacatctagctttgacaatcatagtatgttttattttcctgagggggaataacttataatgctgtttagttttgtactattggtgtgttggtgaatttttaaactgtgtgctaactgcaataaattatatgaactgagaaaaaaaaaaaaaaaaaaaaaaaaaaaaaaaaaaaaaaaaaaaaaaaaa

SEQ ID NO: 245 - DNA, 6 bp, Artificial Sequence
  Description of Artificial Sequence Synthetic oligonucleotide
canntg

SEQ ID NO: 246 - DNA, 6 bp, Artificial Sequence
  Description of Artificial Sequence Synthetic oligonucleotide
cacnag

SEQ ID NO: 247 - protein, 6 aa, Artificial Sequence
  Description of Artificial Sequence Synthetic 6xHis tag
HHHHHH

SEQ ID NO: 248 - RNA, 19 nt, Artificial Sequence
  Description of Artificial Sequence Synthetic oligonucleotide
ccucgcaggucaagagcaa